(19) United States
(12) Patent Application Publication
(10) Pub. No.: US 2025/0333757 A1
(43) Pub. Date: Oct. 30, 2025

(54) RNA MOLECULES FOR TREATING INSECTS

(71) Applicant: Commonwealth Scientific and Industrial Research Organisation, Acton (AU)

(72) Inventors: Ian Kevin Greaves, Richardson (AU); Daai Zhang, Acton (AU); Chengcheng Zhong, Acton (AU); Neil Andrew Smith, Cook (AU); Robert Charles De Feyter, Monash (AU); Ming-Bo Wang, McKellar (AU); Thomas Kieran Walsh, Acton (AU); Lingling Gao, Floreat Park (AU); Baolong Zhang, Nanjing (CN); John Michael Kenneth Roberts, Acton (AU); Biko Kahare Muita, Acton (AU); Kumaran Nagalingam, Dutton Park (AU); Amol Ghodke, Dutton Park (AU); Tess Alexandra James, Dutton Park (AU)

(21) Appl. No.: 18/990,226

(22) Filed: Dec. 20, 2024

(30) Foreign Application Priority Data
Dec. 22, 2023 (AU) 2023904203
Jan. 10, 2024 (AU) 2024900066
Jun. 18, 2024 (AU) 2024901860
Jun. 28, 2024 (AU) 2024902006
Jun. 28, 2024 (AU) 2024902008
Jun. 28, 2024 (AU) 2024902009

(51) Int. Cl. C12N 15/82; C12N 5/10; C12N 15/113; C12N 15/80; C12N 15/85; C12Q 1/6809

(52) U.S. Cl. CPC C12N 15/8271; C12N 5/10; C12N 15/113; C12N 15/80; C12N 15/8509; C12Q 1/6809; C12Q 2600/124

(57) ABSTRACT

The present invention relates to asymmetric RNA molecules, precursors thereof, and their use in gene silencing.

### REFERENCE TO SEQUENCE LISTING

This application incorporates-by-reference nucleotide sequences which are present in the file named “250407_92504_SequenceListing_DH.xml”, which is 501,555 bytes in size, and which was created on Apr. 7, 2025 in the IBM-PC machine format, having an operating system compatibility with MS-Windows, which is contained in the text file filed Apr. 7, 2025 as part of this application.

### FIELD OF THE INVENTION

The present invention relates to asymmetric RNA molecules, precursors thereof, and their use in gene silencing.

### BACKGROUND OF THE INVENTION

RNA silencing is an evolutionarily conserved gene silencing mechanism in eukaryotes that is induced by double-stranded RNA (dsRNA) which may be of a form designated hairpin structured RNA (hpRNA). In the basic RNA silencing pathway, dsRNA is processed by Dicer proteins into short, 20-25 nucleotide (nt) small RNA duplexes, of which one strand is bound to Argonaute (AGO) proteins to form an RNA-induced silencing complex (RISC). This silencing complex uses the small RNA as a guide to find and bind to complementary single-stranded RNA, where the AGO protein cleaves the RNA resulting in its degradation, or translation of the target RNA is reduced without target RNA cleavage.

In plants, multiple RNA silencing pathways exist, including microRNA (miRNA), trans-acting small interfering RNA (tasiRNA), repeat-associated siRNA (rasiRNA) and exogenic (virus and transgene) siRNA (exosiRNA) pathways. miRNAs are 20-24-nt small RNAs processed in the nucleus by Dicer-like 1 (DCL1) from short stem-loop precursor RNAs that are transcribed by RNA polymerase II from MIR genes. tasiRNAs are phased siRNAs of primarily 21 nt in size derived from DCL4 processing of long dsRNA synthesised by RNA-dependent RNA polymerase 6 (RDR6) from miRNA-cleaved TAS RNA fragment. The 24-nt rasiRNAs are produced by DCL3, and the precursor dsRNA is generated by the combined function of plant-specific DNA-dependent RNA polymerase IV (PolIV) and RDR2 from repetitive DNA in the genome. The exosiRNA pathway overlaps with the tasiRNA and rasiRNA pathways and both DCL4 and DCL3 are involved in exosiRNA processing. In addition to DCL1, DCL3 and DCL4, the model plant Arabidopsis thaliana and other higher plants encodes DCL2 or equivalent, which generates 22-nt siRNAs including 22-nt exosiRNAs, and plays a key role in systemic and transitive gene silencing in plants. All of these plant small RNAs are methylated at the 2′-hydroxyl group of 3′ terminal nucleotide by HUA Enhancer 1 (HEN1), and this 3′ terminal 2′-O-methylation is thought to stabilise the small RNAs in plant cells. miRNAs, tasiRNAs and exosiRNAs are functionally similar to small RNAs in animal cells which are involved in posttranscriptional gene silencing or sequence-specific degradation of RNA in animals. The rasiRNAs, however, are unique to plants and function to direct de novo cytosine methylation at the cognate DNA, a transcriptional gene silencing mechanism known as RNA-directed DNA methylation (RdDM).

RNA silencing induced by dsRNA has been extensively exploited to reduce gene activity in various eukaryotic systems, and a number of gene silencing technologies have been developed. Different organisms are often amenable to different gene silencing approaches. For instance, long dsRNA (at least 100 basepairs in length) is less suited to inducing RNA silencing in mammalian cells due to dsRNA-induced interferon responses, and so shorter dsRNAs (less than 30 basepairs) are generally used in mammalian cells, whereas in plants, hairpin RNA (hpRNA) with a long dsRNA stem is highly effective. In plants, the different RNA silencing pathways have led to different gene silencing technologies, such as artificial miRNA, artificial tasiRNA and virus-induced gene silencing technologies. However, successful applications of RNA silencing in plants have so far been achieved primarily by using long hpRNA transgenes. A hpRNA transgene construct typically consists of an inverted repeat made up of fully complementary sense and antisense sequences of a target gene sequence (which when transcribed form the dsRNA stem of hpRNA) separated by a spacer sequence (forming the loop of hpRNA), which is inserted between a promoter and a transcription terminator for expression in plant cells. The spacer sequence functions to stabilise the inverted-repeat DNA in bacteria during construct preparation. The dsRNA stem of the resulting hpRNA transcript is processed by DCL proteins into siRNAs that direct target gene silencing. hpRNA transgenes have been widely used to knock down gene expression, modify metabolic pathways and enhance disease and pest resistance in plants for crop improvement, and many successful applications of the technology in crop improvement have now been reported (Guo et al., 2016; Kim et al., 2019).

WO2019/051563 discloses RNA molecules having double-stranded structures and their use in gene silencing, including a double hairpin structure. WO2020/024019 discloses double-stranded RNA structures having non-canonical basepairs in the double-stranded RNA region and their use in gene silencing. WO2021/022325 discloses double-stranded RNA molecules for use in modulating flowering in plants.

Whilst dsRNA induced gene silencing has proven to be a valuable tool in altering the phenotype of an organism, there is a need for alternate, preferably improved, dsRNA molecules which can be used for RNA interference (RNAi).

### SUMMARY OF THE INVENTION

The present inventors have identified double-stranded product RNA molecules, and precursor RNA molecules encoding the double-stranded product RNA molecules, with desirable characteristics. These are especially useful for down-regulating gene expression or reducing the amount or activity of one or more target RNA molecules in a sequence-specific manner in a eukaryotic cell. In particular, the precursor RNA molecules and therefore also the double-stranded product RNA molecules have an asymmetric design feature that provides one or more bulged ribonucleotides in the precursor and product RNA molecules, preferably also having a ledRNA structure or comprising multiple non-canonical basepairs, preferably G:U basepairs, in a double-stranded region of the RNA molecules. The precursor RNA molecules may be applied topically to a eukaryotic cell, tissue, organ or organism, or be ingested by an organism such as an insect pest, or be expressed from a polynucleotide that encodes the precursor RNA molecules. The precursor RNA molecules thereby provide improved means to control pests and pathogens such as insect pests, nematodes and fungal and viral pathogens, or to reduce the incidence of, or treat, a disease in a eukaryotic organism.

Examples of the precursor RNA molecules and product RNA molecules of the following aspects and embodiments are illustrated schematically in FIG. 53 herein and the reader is encouraged to view FIG. 53 in concert with the description of the following aspects and embodiments.

In a first aspect, the present invention provides asymmetric precursor RNA molecules which are processed to produce siRNA molecules consisting of 21 nt sense RNA sequences hybridised to 22 nt antisense RNA sequences. Therefore, the present invention provides a precursor RNA molecule (A) comprising at least one double-stranded RNA region (B), wherein:
(i) the double-stranded RNA region (B) comprises:
   (a) a first RNA strand (D) of at least 23 contiguous ribonucleotides which comprises a first RNA sequence (E) of at least 21 contiguous ribonucleotides, and
   (b) a second RNA strand (F) of at least 24 contiguous ribonucleotides which comprises a second RNA sequence (G) of at least 22 contiguous ribonucleotides,
wherein the first RNA strand (D) and second RNA strand (F) are covalently linked by a linking RNA sequence (L),
wherein the first RNA sequence (E) is hybridised to the second RNA sequence (G) by basepairing between at least 20 of the at least 21 contiguous ribonucleotides of the first RNA sequence (E) and at least 20 of the at least 22 contiguous ribonucleotides of the second RNA sequence (G), forming at least a part (C) of the double-stranded RNA region (B),
wherein 1 or 2 ribonucleotides of the at least 22 ribonucleotides of (G) are non-basepaired, and 0 or 1, respectively, ribonucleotides of the at least 21 ribonucleotides of (E) are non-basepaired in the part (C) of the double-stranded RNA region (B), forming one or two or three bulges in the part (C) of the double-stranded RNA region (B),
wherein each of the one or two or three bulges is immediately flanked by ribonucleotides which are basepaired in the part (C) of the double-stranded RNA region (B); and
   (ii) the precursor RNA molecule (A) is capable of being cleaved in a eukaryotic cell by one or more ribonucleases (RNases) to produce one or more double-stranded product RNA molecule(s) (P) consisting of a sense RNA sequence (H) of 21 contiguous ribonucleotides from the first RNA sequence (E) and an antisense RNA sequence (J) of 22 contiguous ribonucleotides from the second RNA strand (F), including at least 20 contiguous ribonucleotides from (G),
wherein the one or more double-stranded product RNA molecule(s) (P) comprise the one or two or three bulges,
wherein 18 or 19 of ribonucleotides 1 to 19 of the sense RNA sequence (H) each basepair with a ribonucleotide of ribonucleotides 1 to 20 of antisense RNA sequence (J) in the one or more double-stranded product RNA molecule(s) (P),
wherein ribonucleotides 20 and 21 of the sense RNA sequence (H) form a 2-ribonucleotide unpaired 3′ overhang and ribonucleotides 21 and 22 of the antisense RNA sequence (J) form a 2-ribonucleotide unpaired 3′ overhang in the one or more double-stranded product RNA molecule(s) (P),
wherein ribonucleotides 1 and 2 of the sense RNA sequence (H) basepair with ribonucleotides 19 and 20, respectively, of the antisense RNA sequence (J) in the one or more double-stranded product RNA molecule(s) (P), and
wherein ribonucleotides 18 and 19 of the sense RNA sequence (H) basepair with ribonucleotides 1 and 2, respectively, of the antisense RNA sequence (J) in the one or more double-stranded product RNA molecule(s) (P). Exemplary RNA molecules of this aspect are illustrated schematically in FIG. 53, panels A to H.

In an embodiment of the first aspect, one or both of ribonucleotides 1 and 2 of the at least 23 contiguous ribonucleotides of the first RNA strand (D) are not basepaired with one or both, respectively, of ribonucleotides 23 and 24 of the at least 24 contiguous ribonucleotides of the second RNA strand (F). Exemplary RNA molecules of this embodiment are illustrated schematically in FIG. 53, panels F to H.

In an embodiment of the first aspect, the first RNA sequence (E) is hybridised to the second RNA sequence (G) by basepairing between at least 20 of the at least 21 contiguous ribonucleotides of the first RNA sequence (E) and at least 20 of the at least 22 contiguous ribonucleotides of the second RNA sequence (G),
wherein 2 ribonucleotides of the at least 22 contiguous ribonucleotides of (G) are non-basepaired and 1 ribonucleotide of the at least 21 contiguous ribonucleotides of the first RNA sequence (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein the one or more double-stranded product RNA molecule(s) (P) comprise the bulges,
wherein one bulge is formed by a mismatched ribonucleotide pair between the first RNA sequence (E) and the second RNA sequence (G) and the second bulge is a single-ribonucleotide bulge, and
wherein all but one of ribonucleotides 1 to 19 of the sense RNA sequence (H) each basepair with a ribonucleotide of ribonucleotides 1 to 20 of antisense RNA sequence (J) in the one or more double-stranded product RNA molecule(s) (P). Exemplary RNA molecules of this embodiment are illustrated schematically in FIG. 53, panels A and F.

In an embodiment of the first aspect, the first RNA sequence (E) is hybridised to the second RNA sequence (G) by basepairing between at least 20 of the at least 21 contiguous ribonucleotides of the first RNA sequence (E) and at least 20 of the at least 22 contiguous ribonucleotides of the second RNA sequence (G),
wherein 2 contiguous ribonucleotides of the at least 22 ribonucleotides of (G) are non-basepaired and 1 ribonucleotide of the at least 21 ribonucleotides of (E) is non-basepaired in the part (C) of the double-stranded RNA region (B), the non-basepaired ribonucleotides forming a bulge in the part (C) of the double-stranded RNA region (B), wherein the bulge is immediately flanked by ribonucleotides which are basepaired in the part (C) of the double-stranded RNA region (B),
wherein the one or more double-stranded product RNA molecule(s) (P) comprise the bulge, and
wherein all but one of ribonucleotides 1 to 19 of the sense RNA sequence (H) each basepair with a ribonucleotide of ribonucleotides 1 to 20 of antisense RNA sequence (J) in the one or more double-stranded product RNA molecule(s). Exemplary RNA molecules of this embodiment are illustrated schematically in FIG. 53, panel B.

In an embodiment of the first aspect, the first RNA sequence (E) is hybridised to the second RNA sequence (G) by basepairing between at least 20 of the at least 21 contiguous ribonucleotides of the first RNA sequence (E) and at least 20 of the at least 22 contiguous ribonucleotides of the second RNA sequence (G),
wherein 2 ribonucleotides of the at least 22 ribonucleotides of (G) are non-basepaired and 1 ribonucleotide of the at least 21 ribonucleotides of (E) is non-basepaired in the part (C) of the double-stranded RNA region (B), the non-basepaired ribonucleotides forming three bulges in the part (C) of the double-stranded RNA region (B),
wherein each bulge is immediately flanked by ribonucleotides which are basepaired in the part (C) of the double-stranded RNA region (B),
wherein each bulge is a single-ribonucleotide bulge,
wherein the one or more double-stranded product RNA molecule(s) (P) comprise the bulges, wherein all but one of ribonucleotides 1 to 19 of the sense RNA sequence (H) each basepair with a ribonucleotide of ribonucleotides 1 to 20 of antisense RNA sequence (J) in the one or more double-stranded product RNA molecule(s). Exemplary RNA molecules of this embodiment are illustrated schematically in FIG. 53, panels C and G.

In an embodiment of the first aspect, the first RNA sequence (E) is hybridised to the second RNA sequence (G) by basepairing between at least 21 of the at least 21 contiguous ribonucleotides of the first RNA sequence (E) and at least 21 of the at least 22 contiguous ribonucleotides of the second RNA sequence (G),
wherein 1 ribonucleotide of the at least 22 ribonucleotides of (G) is non-basepaired and all of the at least 21 ribonucleotides of (E) are basepaired in the part (C) of the double-stranded RNA region (B), the 1 non-basepaired ribonucleotide forming a single nucleotide bulge in the part (C) of the double-stranded RNA region (B),
wherein the bulge is immediately flanked by ribonucleotides which are basepaired in the part (C) of the double-stranded RNA region (B),
wherein the one or more double-stranded product RNA molecule(s) (P) comprise the bulge, and
wherein ribonucleotides 1 to 19 of the sense RNA sequence (H) each basepair with a ribonucleotide of ribonucleotides 1 to 20 of antisense RNA sequence (J) in the one or more double-stranded product RNA molecule(s) (P). Exemplary RNA molecules of this embodiment are illustrated schematically in FIG. 53, panels D, E and H.

In an embodiment of the first aspect, the double-stranded RNA region (B) comprises:
(a) a first RNA strand (D) which comprises a first RNA sequence (E) of at least 23 contiguous ribonucleotides, and
(b) a second RNA strand (F) which comprises a second RNA sequence (G) of at least 24 contiguous ribonucleotides,
wherein the first RNA sequence (E) is hybridised to the second RNA sequence (G) by basepairing between at least 22 of the at least 23 contiguous ribonucleotides of the first RNA sequence (E) and at least 22 of the at least 24 contiguous ribonucleotides of the second RNA sequence (G), forming at least a part (C) of the double-stranded RNA region (B),
wherein 1 or 2 ribonucleotides of the at least 24 ribonucleotides of (G) are non-basepaired and 0 or 1, respectively, ribonucleotides of the at least 23 ribonucleotides of (E) are non-basepaired in the part (C) of the double-stranded RNA region (B), forming one or two or three bulges in the part (C) of the double-stranded RNA region (B). Exemplary RNA molecules of this embodiment are illustrated schematically in FIG. 53, panels A to E.

Thus, in an embodiment the present invention provides a precursor RNA molecule (A) comprising at least one double-stranded RNA region (B), wherein:
(i) the double-stranded RNA region (B) comprises:
   (a) a first RNA strand (D) which comprises a first RNA sequence (E) of at least 23 contiguous ribonucleotides, and
   (b) a second RNA strand (F) which comprises a second RNA sequence (G) of at least 24 contiguous ribonucleotides,
wherein the first RNA strand (D) and second RNA strand (F) are covalently linked by a linking RNA sequence (L),
wherein the first RNA sequence (E) is hybridised to the second RNA sequence (G) by basepairing between at least 22 of the at least 23 contiguous ribonucleotides of the first RNA sequence (E) and at least 22 of the at least 24 contiguous ribonucleotides of the second RNA sequence (G), forming at least a part (C) of the double-stranded RNA region (B),
wherein 1 or 2 ribonucleotides of the at least 24 ribonucleotides of (G) are non-basepaired, and 0 or 1, respectively, ribonucleotides of the at least 23 ribonucleotides of (E) are non-basepaired, in the part (C) of the double-stranded RNA region (B) forming one or two or three bulges in the part (C) of the double-stranded RNA region (B),
wherein each of the one or two or three bulges is immediately flanked by ribonucleotides which are basepaired in the part (C) of the double-stranded RNA region (B); and
   (ii) the precursor RNA molecule (A) is capable of being cleaved in a eukaryotic cell by one or more ribonucleases (RNases) to produce one or more double-stranded product RNA molecule(s) (P) consisting of a sense RNA sequence (H) of 21 contiguous ribonucleotides from the first RNA sequence (E) and an antisense RNA sequence (J) of 22 contiguous ribonucleotides from the second RNA sequence (G),
wherein the one or more double-stranded product RNA molecule(s) (P) comprise the one or two or three bulges,
wherein 18 or 19 of ribonucleotides 1 to 19 of the sense RNA sequence (H) each basepair with a ribonucleotide of ribonucleotides 1 to 20 of antisense RNA sequence (J) in the one or more double-stranded product RNA molecule(s) (P),
wherein ribonucleotides 20 and 21 of the sense RNA sequence (H) form a 2-ribonucleotide unpaired 3′ overhang and ribonucleotides 21 and 22 of the antisense RNA sequence (J) form a 2-ribonucleotide unpaired 3′ overhang in the one or more double-stranded product RNA molecule(s) (P),
wherein ribonucleotides 1 and 2 of the sense RNA sequence (H) basepair with ribonucleotides 19 and 20, respectively, of the antisense RNA sequence (J) in the one or more double-stranded product RNA molecule(s) (P), and
wherein ribonucleotides 18 and 19 of the sense RNA sequence (H) basepair with ribonucleotides 1 and 2, respectively, of the antisense RNA sequence (J) in the one or more double-stranded product RNA molecule(s) (P). Exemplary RNA molecules of this embodiment are illustrated schematically in FIG. 53, panels A to E.

In an embodiment of the first aspect, the first RNA sequence (E) is hybridised to the second RNA sequence (G) by basepairing between at least 22 of the at least 23 contiguous ribonucleotides of the first RNA sequence (E) and at least 22 of the at least 24 contiguous ribonucleotides of the second RNA sequence (G),
wherein 2 ribonucleotides of the at least 24 ribonucleotides of (G) are non-basepaired and 1 ribonucleotide of the at least 23 ribonucleotides of (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein the one or more double-stranded product RNA molecule(s) (P) comprise the bulges,
wherein one bulge is formed by a mismatched ribonucleotide pair between the first RNA sequence (E) and the second RNA sequence (G) and the second bulge is a single-ribonucleotide bulge, and
wherein all but one of ribonucleotides 1 to 19 of the sense RNA sequence (H) each basepair with a ribonucleotide of ribonucleotides 1 to 20 of antisense RNA sequence (J) in the one or more double-stranded product RNA molecule(s) (P). Exemplary RNA molecules of this embodiment are illustrated schematically in FIG. 53, panel A.

In an embodiment of the first aspect, the first RNA sequence (E) is hybridised to the second RNA sequence (G) by basepairing between at least 22 of the at least 23 contiguous ribonucleotides of the first RNA sequence (E) and at least 22 of the at least 24 contiguous ribonucleotides of the second RNA sequence (G),
wherein 2 contiguous ribonucleotides of the at least 24 ribonucleotides of (G) are non-basepaired and 1 ribonucleotide of the at least 23 ribonucleotides of (E) is non-basepaired in the part (C) of the double-stranded RNA region (B), the non-basepaired ribonucleotides forming a bulge in the part (C) of the double-stranded RNA region (B),
wherein the bulge is immediately flanked by ribonucleotides which are basepaired in the part (C) of the double-stranded RNA region (B),
wherein the one or more double-stranded product RNA molecule(s) (P) comprise the bulge, and
wherein all but one of ribonucleotides 1 to 19 of the sense RNA sequence (H) each basepair with a ribonucleotide of ribonucleotides 1 to 20 of antisense RNA sequence (J) in the one or more double-stranded product RNA molecule(s). Exemplary RNA molecules of this embodiment are illustrated schematically in FIG. 53, panel B.

In an embodiment of the first aspect, the first RNA sequence (E) is hybridised to the second RNA sequence (G) by basepairing between at least 22 of the at least 23 contiguous ribonucleotides of the first RNA sequence (E) and at least 22 of the at least 24 contiguous ribonucleotides of the second RNA sequence (G),
wherein 2 ribonucleotides of the at least 24 ribonucleotides of (G) are non-basepaired and 1 ribonucleotide of the at least 23 ribonucleotides of (E) is non-basepaired in the part (C) of the double-stranded RNA region (B), the non-basepaired ribonucleotides forming three bulges in the part (C) of the double-stranded RNA region (B),
wherein each bulge is immediately flanked by ribonucleotides which are basepaired in the part (C) of the double-stranded RNA region (B),
wherein each bulge is a single-ribonucleotide bulge,
wherein the one or more double-stranded product RNA molecule(s) (P) comprise the bulges, wherein all but one of ribonucleotides 1 to 19 of the sense RNA sequence (H) each basepair with a ribonucleotide of ribonucleotides 1 to 20 of antisense RNA sequence (J) in the one or more double-stranded product RNA molecule(s). Exemplary RNA molecules of this embodiment are illustrated schematically in FIG. 53, panel C.

In an embodiment of the first aspect, the first RNA sequence (E) is hybridised to the second RNA sequence (G) by basepairing between at least 23 of the at least 23 contiguous ribonucleotides of the first RNA sequence (E) and at least 23 of the at least 24 contiguous ribonucleotides of the second RNA sequence (G),
wherein 1 ribonucleotide of the at least 24 ribonucleotides of (G) is non-basepaired and all of the at least 23 ribonucleotides of (E) are basepaired in the part (C) of the double-stranded RNA region (B), the 1 non-basepaired ribonucleotide forming a single nucleotide bulge in the part (C) of the double-stranded RNA region (B),
wherein the bulge is immediately flanked by ribonucleotides which are basepaired in the part (C) of the double-stranded RNA region (B),
wherein the one or more double-stranded product RNA molecule(s) (P) comprise the bulge, and
wherein ribonucleotides 1 to 19 of the sense RNA sequence (H) each basepair with a ribonucleotide of ribonucleotides 1 to 20 of antisense RNA sequence (J) in the one or more double-stranded product RNA molecule(s) (P). Exemplary RNA molecules of this embodiment are illustrated schematically in FIG. 53, panels D and E.

The design principles for the asymmetric precursor RNA molecules of the first aspect can be applied in an extended fashion to longer double-stranded regions. For example, the non-basepaired ribonucleotides in the second RNA sequence (G) (antisense sequence) may be arranged in a periodic fashion to provide a population of product RNA molecules (P) having multiple, non-overlapping antisense RNA sequences (J) of 22 nt. Such precursor RNA molecules are particularly useful for reducing expression of a target RNA molecule in a plant cell, fungal cell or nematode cell. They are also useful in other invertebrate animal cells such as an arthropod cell or insect cell, or in a non-mammalian vertebrate animal cell. They may be produced in a plant cell to reduce an insect target RNA molecule or a fungal pathogen or nematode target RNA molecule, or applied topically to a plant or insect to reduce a target RNA molecule. For example, in an embodiment of the first aspect, the first RNA sequence (E) comprises at least 44 contiguous ribonucleotides and the second RNA sequence (G) comprises at least 46 contiguous ribonucleotides,
wherein the first RNA sequence (E) is hybridised to the second RNA sequence (G) by basepairing between at least 42, preferably at least 43 or at least 44, of the at least 44 contiguous ribonucleotides of the first RNA sequence (E) and at least 42, preferably at least 43 or at least 44, of the at least 46 contiguous ribonucleotides of the second RNA sequence (G),
wherein 2, 3 or 4 ribonucleotides of the at least 46 contiguous ribonucleotides of (G) are non-basepaired and 0, 1 or 2, respectively, ribonucleotides of the at least 44 contiguous ribonucleotides of (E) are non-basepaired in the part (C) of the double-stranded RNA region (B), forming bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 22 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J).

In another embodiment of the first aspect, the first RNA strand (D) comprises a first RNA sequence (E) of at least 44 contiguous ribonucleotides and the second RNA strand (F) comprises a second RNA sequence (G) of at least 46 contiguous ribonucleotides,
wherein the first RNA sequence (E) is hybridised to the second RNA sequence (G) by basepairing between all 44 contiguous ribonucleotides of the first RNA sequence (E) and 44 of the 46 contiguous ribonucleotides of the second RNA sequence (G), forming a part (C) of the double-stranded RNA region (B),
wherein 2 ribonucleotides of the 46 contiguous ribonucleotides of (G) are non-basepaired in the part (C) of the double-stranded RNA region (B), forming single ribonucleotide bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 22 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the single ribonucleotide bulges are preferably spaced apart by 16-26 contiguous basepairs, more preferably by 17-24 or 18-23 contiguous basepairs, even more preferably by 19-22 contiguous basepairs, most preferably by 20 or 21 contiguous basepairs. In specific examples, the single ribonucleotide bulges are spaced apart by 16, 17, 18, 19, 20, 21, 22, 23, 24, 25 or 26 contiguous basepairs. In an example, all of these basepairs are canonical basepairs. Alternatively, 1, 2, 3, 4, 5 or 6 of the basepairs between the bulges are G:U basepairs. The precursor RNA molecule may comprise two or more such double-stranded RNA regions, for example comprise an ledRNA structure. In these embodiments, the eukaryotic cell in which the precursor RNA molecule (A) is cleaved is preferably a plant cell, or a fungal cell, or a nematode cell, or an arthropod cell such as an insect cell, arachnid, or decapod, or the target RNA molecule is preferably in a plant cell, or a fungal cell, or a nematode cell, or an arthropod cell such as an insect, arachnid, or decapod cell. The precursor RNA molecule may be produced in a cell-free system or in a microbial cell such as a bacterial cell or yeast cell, and such cells applied topically to the plant or insect or are ingested by the insect.

In a different embodiment with longer dsRNA regions of at least 44 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 44 contiguous ribonucleotides and the second RNA strand (F) comprises a second RNA sequence (G) of at least 46 contiguous ribonucleotides,
wherein the first RNA sequence (E) is hybridised to the second RNA sequence (G) by basepairing between all 44 contiguous ribonucleotides of the first RNA sequence (E) and 44 of the 46 contiguous ribonucleotides of the second RNA sequence (G), forming a part (C) of the double-stranded RNA region (B),
wherein 2 ribonucleotides of the 46 contiguous ribonucleotides of (G) are non-basepaired in the part (C) of the double-stranded RNA region (B), forming single ribonucleotide bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 22 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the single ribonucleotide bulges are preferably spaced apart by 16-26 contiguous basepairs, more preferably by 17-24 or 18-23 contiguous basepairs, even more preferably by 19-22 contiguous basepairs, most preferably by 20 or 21 contiguous basepairs. In specific examples, the single ribonucleotide bulges are spaced apart by 16, 17, 18, 19, 20, 21, 22, 23, 24, 25 or 26 contiguous basepairs. In a preferred example, all of these basepairs are canonical basepairs. Alternatively, 1, 2, 3, 4, 5 or 6 of the basepairs between the bulges are G:U basepairs.

In the previous three embodiments, the first RNA sequence (E) and the second RNA sequence (G) may extend to longer than 44 and 46 ribonucleotides, respectively. In another embodiment of the first aspect, for even longer dsRNA region(s), the first RNA sequence (E) comprises at least 65 contiguous ribonucleotides and the second RNA sequence (G) comprises at least 68 contiguous ribonucleotides, and wherein the first RNA sequence (E) is hybridised to the second RNA sequence (G) by basepairing between at least 62, preferably at least 63, at least 64 or at least 65, of the at least 65 contiguous ribonucleotides of the first RNA sequence (E) and at least 62, preferably at least 63, at least 64 or at least 65, of the at least 68 contiguous ribonucleotides of the second RNA sequence (G).

In another embodiment of the first aspect, the first RNA strand (D) comprises a first RNA sequence (E) of at least 65 contiguous ribonucleotides and the second RNA strand (F) comprises a second RNA sequence (G) of at least 68 contiguous ribonucleotides,
wherein the first RNA sequence (E) is hybridised to the second RNA sequence (G) by basepairing between all 65 contiguous ribonucleotides of the first RNA sequence (E) and 65 of the 68 contiguous ribonucleotides of the second RNA sequence (G), forming a part (C) of the double-stranded RNA region (B),
wherein 3 ribonucleotides of the 68 contiguous ribonucleotides of (G) are non-basepaired in the part (C) of the double-stranded RNA region (B), forming single ribonucleotide bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 22 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the single ribonucleotide bulges are preferably spaced apart periodically, for example by 16-26 contiguous basepairs, more preferably by 17-24 or 18-23 contiguous basepairs, even more preferably by 19-22 contiguous basepairs, most preferably by 20 or 21 contiguous basepairs. In specific examples, the single ribonucleotide bulges are spaced apart by 16, 17, 18, 19, 20, 21, 22, 23, 24, 25 or 26 contiguous basepairs. In an example, all of these basepairs are canonical basepairs. Alternatively, 1, 2, 3, 4, 5 or 6 of the basepairs between the bulges are G:U basepairs. The precursor RNA molecule may comprise two or more such double-stranded RNA regions, for example comprise an ledRNA structure.

In a different embodiment with longer dsRNA regions of at least 65 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 65 contiguous ribonucleotides and the second RNA strand (F) comprises a second RNA sequence (G) of at least 68 contiguous ribonucleotides,
wherein the first RNA sequence (E) is hybridised to the second RNA sequence (G) by basepairing between all 65 contiguous ribonucleotides of the first RNA sequence (E) and 65 of the 68 contiguous ribonucleotides of the second RNA sequence (G), forming a part (C) of the double-stranded RNA region (B),
wherein 3 ribonucleotides of the 68 contiguous ribonucleotides of (G) are non-basepaired in the part (C) of the double-stranded RNA region (B), forming single ribonucleotide bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 22 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the single ribonucleotide bulges are preferably spaced apart by 16-26 contiguous basepairs, more preferably by 17-24 or 18-23 contiguous basepairs, even more preferably by 19-22 contiguous basepairs, most preferably by 20 or 21 contiguous basepairs. In specific examples, the single ribonucleotide bulges are spaced apart by 16, 17, 18, 19, 20, 21, 22, 23, 24, 25 or 26 contiguous basepairs. In a preferred example, all of these basepairs are canonical basepairs. Alternatively, 1, 2, 3, 4, 5 or 6 of the basepairs between the bulges are G:U basepairs.

In the previous three embodiments, the first RNA sequence (E) and the second RNA sequence (G) may extend to longer than 65 and 68 ribonucleotides, respectively. In a further embodiment of the first aspect, the first RNA sequence (E) comprises at least 86 contiguous ribonucleotides and the second RNA sequence (G) comprises at least 90 contiguous ribonucleotides, and wherein the first RNA sequence (E) is hybridised to the second RNA sequence (G) by basepairing between at least 82, preferably at least 83, at least 84, at least 85 or at least 86, of the at least 86 contiguous ribonucleotides of the first RNA sequence (E) and at least 82, preferably at least 83, at least 84, at least 85 or at least 86, of the at least 90 contiguous ribonucleotides of the second RNA sequence (G).

In another embodiment of the first aspect, the first RNA strand (D) comprises a first RNA sequence (E) of at least 86 contiguous ribonucleotides and the second RNA strand (F) comprises a second RNA sequence (G) of at least 90 contiguous ribonucleotides,
wherein the first RNA sequence (E) is hybridised to the second RNA sequence (G) by basepairing between all 86 contiguous ribonucleotides of the first RNA sequence (E) and 86 of the 90 contiguous ribonucleotides of the second RNA sequence (G), forming a part (C) of the double-stranded RNA region (B),
wherein 4 ribonucleotides of the 90 contiguous ribonucleotides of (G) are non-basepaired in the part (C) of the double-stranded RNA region (B), forming single ribonucleotide bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 22 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the single ribonucleotide bulges are preferably spaced apart periodically, for example by 16-26 contiguous basepairs, more preferably by 17-24 or 18-23 contiguous basepairs, even more preferably by 19-22 contiguous basepairs, most preferably by 20 or 21 contiguous basepairs or, on average, by about 21 basepairs. In specific examples, the single ribonucleotide bulges are spaced apart, independently, by 16, 17, 18, 19, 20, 21, 22, 23, 24, 25 or 26 contiguous basepairs. In an example, all of these basepairs are canonical basepairs. Alternatively, 1, 2, 3, 4, 5 or 6 of the basepairs between the bulges are G:U basepairs. The precursor RNA molecule may comprise two or more such double-stranded RNA regions, for example comprise a ledRNA structure.

In a different embodiment with longer dsRNA regions of at least 86 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 86 contiguous ribonucleotides and the second RNA strand (F) comprises a second RNA sequence (G) of at least 90 contiguous ribonucleotides,
wherein the first RNA sequence (E) is hybridised to the second RNA sequence (G) by basepairing between all 86 contiguous ribonucleotides of the first RNA sequence (E) and 86 of the 90 contiguous ribonucleotides of the second RNA sequence (G), forming a part (C) of the double-stranded RNA region (B),
wherein 4 ribonucleotides of the 86 contiguous ribonucleotides of (G) are non-basepaired in the part (C) of the double-stranded RNA region (B), forming single ribonucleotide bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 22 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the single ribonucleotide bulges are preferably spaced apart by 16-26 contiguous basepairs, more preferably by 17-24 or 18-23 contiguous basepairs, even more preferably by 19-22 contiguous basepairs, most preferably by 20 or 21 contiguous basepairs, or, on average, about 21 basepairs. In specific examples, the single ribonucleotide bulges are spaced apart, independently, by 16, 17, 18, 19, 20, 21, 22, 23, 24, 25 or 26 contiguous basepairs. In a preferred example, all of these basepairs are canonical basepairs. Alternatively, 1, 2, 3, 4, 5 or 6 of the basepairs between the bulges are G:U basepairs.

In the previous three embodiments, the first RNA sequence (E) and the second RNA sequence (G) may extend to longer than 86 and 90 ribonucleotides, respectively. In a further embodiment of the first aspect, the first RNA sequence (E) comprises at least 107 contiguous ribonucleotides and the second RNA sequence (G) comprises at least 112 contiguous ribonucleotides, and wherein the first RNA sequence (E) is hybridised to the second RNA sequence (G) by basepairing between at least 102, preferably at least 103, at least 104, at least 105, at least 106 or at least 107, of the at least 107 contiguous ribonucleotides of the first RNA sequence (E) and at least 102, preferably at least 103, at least 104, at least 105, at least 106 or at least 107, of the at least 112 contiguous ribonucleotides of the second RNA sequence (G). In this embodiment, at least 5 ribonucleotides of the second RNA sequence, up to a maximum of 10 ribonucleotides, are non-basepaired and form bulges, preferably single ribonucleotide bulges.

In another embodiment of the first aspect, the first RNA strand (D) comprises a first RNA sequence (E) of at least 107 contiguous ribonucleotides and the second RNA strand (F) comprises a second RNA sequence (G) of at least 112 contiguous ribonucleotides,
wherein the first RNA sequence (E) is hybridised to the second RNA sequence (G) by basepairing between all 107 contiguous ribonucleotides of the first RNA sequence (E) and 107 of the 112 contiguous ribonucleotides of the second RNA sequence (G), forming a part (C) of the double-stranded RNA region (B),
wherein 5 ribonucleotides of the 112 contiguous ribonucleotides of (G) are non-basepaired in the part (C) of the double-stranded RNA region (B), forming single ribonucleotide bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 22 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the single ribonucleotide bulges are preferably spaced apart periodically, for example by 16-26 contiguous basepairs, more preferably by 17-24 or 18-23 contiguous basepairs, even more preferably by 19-22 contiguous basepairs, most preferably by 20 or 21 contiguous basepairs or, on average, by about 21 basepairs. In specific examples, the single ribonucleotide bulges are spaced apart, independently, by 16, 17, 18, 19, 20, 21, 22, 23, 24, 25 or 26 contiguous basepairs. In an example, all of these basepairs are canonical basepairs. Alternatively, 1, 2, 3, 4, 5 or 6 of the basepairs between the bulges are G:U basepairs. The precursor RNA molecule may comprise two or more such double-stranded RNA regions, for example comprise an ledRNA structure.

In a different embodiment with longer dsRNA regions of at least 107 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 107 contiguous ribonucleotides and the second RNA strand (F) comprises a second RNA sequence (G) of at least 112 contiguous ribonucleotides,
wherein the first RNA sequence (E) is hybridised to the second RNA sequence (G) by basepairing between all 107 contiguous ribonucleotides of the first RNA sequence (E) and 107 of the 112 contiguous ribonucleotides of the second RNA sequence (G), forming a part (C) of the double-stranded RNA region (B),
wherein 5 ribonucleotides of the 112 contiguous ribonucleotides of (G) are non-basepaired in the part (C) of the double-stranded RNA region (B), forming single ribonucleotide bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 22 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the single ribonucleotide bulges are preferably spaced apart by 16-26 contiguous basepairs, more preferably by 17-24 or 18-23 contiguous basepairs, even more preferably by 19-22 contiguous basepairs, most preferably by 20 or 21 contiguous basepairs, or, on average, about 21 basepairs. In specific examples, the single ribonucleotide bulges are spaced apart, independently, by 16, 17, 18, 19, 20, 21, 22, 23, 24, 25 or 26 contiguous basepairs. In a preferred example, all of these basepairs are canonical basepairs. Alternatively, 1, 2, 3, 4, 5 or 6 of the basepairs between the bulges are G:U basepairs

In each of the above embodiments, the second RNA sequence (G) is longer than the first RNA sequence (E) because of the non-basepaired ribonucleotides that bulge out in (G). Preferably, the length of the first RNA sequence (E) is 94%-97% or 94%-96% of the length of the second RNA sequence (G). These features may also apply across the full length of the double-stranded region (B), where the part (C) of the double-stranded region is the full length of (B).

As the skilled person would appreciate, further embodiments include a first RNA sequence (E) hybridised to a second RNA sequence (G) having longer contiguous ribonucleotides following the same principles described in the above embodiments.

In an embodiment, the first RNA sequence (E) and the second RNA sequence (G) both comprise at least 100 contiguous ribonucleotides, or at least 150, or at least 200, or at least 250, or at least 300 contiguous ribonucleotides, preferably to a maximum of 1000 contiguous ribonucleotides, more preferably to a maximum of 800 contiguous ribonucleotides, or even more preferably to a maximum of 600 contiguous ribonucleotides. For example, the first RNA sequence (E) and the second RNA sequence (G) both comprise contiguous ribonucleotides in the range 100-1000, 100-800, or 100-600 contiguous ribonucleotides, or in the range 150-1000, 150-800, or 150-600 contiguous ribonucleotides. In preferred embodiments, the length of the sense RNA sequence of the dsRNA region is 94%-97% or 94%-96% the length of the antisense sequence. These features are applicable to hairpin RNAs and to dsRNAs formed by annealing of two RNA strands, i.e. without a joining loop sequence. Each of these features may also be applied to a second dsRNA region in the precursor RNA molecule, for example in a ledRNA molecule. In these embodiments, the eukaryotic cell in which the precursor RNA molecule (A) is cleaved is preferably a plant cell, a fungal cell, a nematode cell, or an arthropod cell such as an insect, arachnid, or decapod cell, or the target RNA molecule is preferably in a plant cell, a fungal cell, a nematode cell, or an arthropod cell such as an insect, arachnid, or decapod cell.

Each of the embodiments of the first aspect may have the following feature:
(i) ribonucleotides 1, 2 and 3 of the sense RNA sequence (H) basepair with ribonucleotides 18, 19 and 20, respectively, of the antisense RNA sequence (J) in at least some of the one or more double-stranded product RNA molecule(s) (P), or
(ii) ribonucleotides 17, 18 and 19 of the sense RNA sequence (H) basepair with ribonucleotides 1, 2 and 3, respectively, of the antisense RNA sequence (J) in at least some of the one or more double-stranded product RNA molecule(s) (P), or
preferably both (i) and (ii). Exemplary RNA molecules of this embodiment are illustrated schematically in FIG. 53, panels A and H, which all show product RNA molecules with at least three contiguous basepairs at both ends of the double-stranded region of the product RNA molecules. In further features of these embodiments, the product RNA molecules may have at least four, at least five or at least six contiguous basepairs at one or both ends of the double-stranded molecule before any bulge. In these embodiments, any of those basepairs may be G:U basepairs, preferably one or more of ribonucleotides 1, 2 and 3 of the antisense sequence (J) are involved in G:U basepairs, more preferably one or both of ribonucleotides 1 and 2 of the antisense sequence (J) are involved in G:U basepairs, even more preferably ribonucleotide 1 of the antisense sequence (J) is involved in G:U basepairs. Most preferably, one or more of ribonucleotides 1, 2 and 3 of the antisense sequence (J) are U ribonucleotides, or one or both of ribonucleotides 1 and 2 are U ribonucleotides, or ribonucleotide 1 is a U ribonucleotide, involved in a G:U basepair.

Each of the features of the embodiments of the first aspect can be applied to longer double-stranded regions to provide essentially either multimers of the product RNA molecules (P) or combinations of different designs of product RNA molecules (P). In an embodiment of the first aspect, the precursor RNA molecule is capable of being cleaved in a eukaryotic cell by one or more ribonucleases (RNases) to produce multiple, different double-stranded product RNA molecules (P) which each consist of a sense RNA sequence (H) of 21 contiguous ribonucleotides from the first RNA sequence (E) and an antisense RNA sequence (J) of 22 contiguous ribonucleotides from the second RNA sequence (G), wherein at least some of the multiple, different double-stranded product RNA molecules (P) have overlapping antisense RNA sequences (J). In a further embodiment, at least some of the multiple, different double-stranded product RNA molecules (P) have non-overlapping antisense RNA sequences (J), where the population of double-stranded product RNA molecules (P) produced from the precursor RNA molecule includes some overlapping and some non-overlapping antisense RNA sequences (J). Preferably, there are more non-overlapping antisense RNA sequences (J), as readily occurs with longer (>42 basepairs) double-stranded regions (B) where siRNA molecules each consisting of 21 nt sense RNA sequences hybridised to 22 nt antisense RNA sequences are phased along the length of (B).

In an embodiment of the first aspect, the precursor RNA molecule is capable of being cleaved in a eukaryotic cell by one or more ribonucleases (RNases) to produce multiple, different double-stranded product RNA molecules (P) which each consist of a sense RNA sequence (H) of 21 contiguous ribonucleotides from a first RNA sequence (E) and an antisense RNA sequence (J) of 22 contiguous ribonucleotides from a second RNA sequence (G), wherein at least some of the multiple, different double-stranded product RNA molecules (P) have non-overlapping antisense RNA sequences (J), preferably adjacent non-overlapping antisense RNA sequences (J). This readily occurs with longer double-stranded regions (B) where the siRNA molecules each consisting of 21 nt sense RNA sequences hybridised to 22 nt antisense RNA sequences are repeated along the length of (B), where phased cleavage by Dicer can occur.

In an embodiment of the first aspect, the precursor RNA molecule is capable of being cleaved in a eukaryotic cell by one or more ribonucleases (RNases) to produce multiple, different double-stranded product RNA molecules (P) which comprise double-stranded RNA product molecules as defined in two or more of the above embodiments.

Each of the embodiments of the first aspect may have the following feature: at least some of the one or more double-stranded product RNA molecule(s) (P) comprise one or two or three non-basepaired ribonucleotide(s) selected from the group consisting of ribonucleotides 4, 5, 6, 7, 8, 9, 10, 11, 12, 13, 14, 15, 16 and 17 of the antisense RNA sequence (J).

Each of the embodiments of the first aspect may have the following feature: at least some of the one or more double-stranded product RNA molecule(s) (P) comprise one or two non-basepaired ribonucleotide(s), preferably one non-basepaired ribonucleotide, selected from the group consisting of ribonucleotides 4, 5, 6, 7, 8, 9, 10, 11, 12, 13, 14, 15, 16 and 17 of the antisense RNA sequence (J).

In a second aspect, the present invention provides asymmetric precursor RNA molecules which are processed to produce siRNA molecules consisting of 21 nt sense RNA sequences hybridised to 23 nt antisense RNA sequences. Therefore, in this aspect the present invention provides a precursor RNA molecule (A) comprising at least one double-stranded RNA region (B), wherein:
(i) the double-stranded RNA region (B) comprises:
   (a) a first RNA strand (D) of at least 23 contiguous ribonucleotides which comprises a first RNA sequence (E) of at least 21 contiguous ribonucleotides, and
   (b) a second RNA strand (F) of at least 25 contiguous ribonucleotides which comprises a second RNA sequence (G) of at least 23 contiguous ribonucleotides,
wherein the first RNA strand (D) and second RNA strand (F) are covalently linked by a linking RNA sequence (L),
wherein the first RNA sequence (E) is hybridised to the second RNA sequence (G) by basepairing between at least 20 of the at least 21 contiguous ribonucleotides of the first RNA sequence (E) and at least 20 of the at least 23 contiguous ribonucleotides of the second RNA sequence (G), forming at least a part (C) of the double-stranded RNA region (B),
wherein 2 or 3 ribonucleotides of the at least 23 ribonucleotides of (G) are non-basepaired and 0 or 1, respectively, ribonucleotides of the at least 21 ribonucleotides of (E) are non-basepaired in the part (C) of the double-stranded RNA region (B), forming one, two, three or four bulges in the part (C) of the double-stranded RNA region (B),
wherein each of the one, two, three or four bulges is immediately flanked by ribonucleotides which are basepaired in the part (C) of the double-stranded RNA region (B); and
   (ii) the precursor RNA molecule (A) is capable of being cleaved in a eukaryotic cell by one or more ribonucleases (RNases) to produce one or more double-stranded product RNA molecule(s) (P) consisting of a sense RNA sequence (H) of 21 contiguous ribonucleotides from the first RNA sequence (E) and an antisense RNA sequence (J) of 23 contiguous ribonucleotides from the second RNA strand (F), including at least 21 contiguous ribonucleotides from (G),
wherein the one or more double-stranded product RNA molecule(s) (P) comprise the one, two, three or four bulges,
wherein 18 or 19 of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P),
wherein ribonucleotides 20 and 21 of the sense RNA sequence (H) form a 2-ribonucleotide unpaired 3′ overhang and ribonucleotides 22 and 23 of the antisense RNA sequence (J) form a 2-ribonucleotide unpaired 3′ overhang in the one or more double-stranded product RNA molecule(s) (P),
wherein ribonucleotides 1 and 2 of the sense RNA sequence (H) basepair with ribonucleotides 20 and 21, respectively, of the antisense RNA sequence (J) in the one or more double-stranded product RNA molecule(s) (P), and
wherein ribonucleotides 18 and 19 of the sense RNA sequence (H) basepair with ribonucleotides 1 and 2, respectively, of the antisense RNA sequence (J) in the one or more double-stranded product RNA molecule(s) (P). Exemplary RNA molecules of this aspect are illustrated schematically in FIG. 53, panels I to Q.

In an embodiment of the second aspect, one or both of ribonucleotides 1 and 2 of the at least 23 contiguous ribonucleotides of the first RNA strand (D) are not basepaired with one or both, respectively, of ribonucleotides 24 and 25 of the at least 25 contiguous ribonucleotides of the second RNA strand (F). Exemplary RNA molecules of this embodiment are illustrated schematically in FIG. 53, panels O to Q.

In an embodiment of the second aspect, the first RNA sequence (E) is hybridised to the second RNA sequence (G) by basepairing between at least 20 of the at least 21 contiguous ribonucleotides of the first RNA sequence (E) and at least 20 of the at least 23 contiguous ribonucleotides of the second RNA sequence (G),
wherein 3 ribonucleotides, 2 of which are contiguous, of the at least 23 ribonucleotides of (G) are non-basepaired and 1 ribonucleotide of the at least 21 ribonucleotides of (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein one bulge is formed by a mismatched ribonucleotide pair between the first RNA sequence (E) and the second RNA sequence (G) and the second bulge is a di-ribonucleotide bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P). Exemplary RNA molecules of this embodiment are illustrated schematically in FIG. 53, panel I.

In an embodiment of the second aspect, the first RNA sequence (E) is hybridised to the second RNA sequence (G) by basepairing between at least 20 of the at least 21 contiguous ribonucleotides of the first RNA sequence (E) and at least 20 of the at least 23 contiguous ribonucleotides of the second RNA sequence (G),
wherein 3 ribonucleotides of the at least 23 ribonucleotides of (G) are non-basepaired, and 1 ribonucleotide of the at least 21 ribonucleotides of (E) is non-basepaired in the part (C) of the double-stranded RNA region (B), the non-basepaired ribonucleotides forming four single-ribonucleotide bulges in the part (C) of the double-stranded RNA region (B),
wherein each bulge is immediately flanked by ribonucleotides which are basepaired in the part (C) of the double-stranded RNA region (B),
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P). Exemplary RNA molecules of this embodiment are illustrated schematically in FIG. 53, panel J.

In an embodiment of the second aspect, the first RNA sequence (E) is hybridised to the second RNA sequence (G) by basepairing between at least 21 of the at least 21 contiguous ribonucleotides of the first RNA sequence (E) and at least 21 of the at least 23 contiguous ribonucleotides of the second RNA sequence (G),
wherein 2 contiguous ribonucleotides of the at least 23 ribonucleotides of (G) are non-basepaired and all of the at least 21 ribonucleotides of (E) are basepaired in the part (C) of the double-stranded RNA region (B), the 2 non-basepaired ribonucleotides forming a di-ribonucleotide bulge, or two single-ribonucleotide bulges, in the part (C) of the double-stranded RNA region (B),
wherein the bulge is immediately flanked by ribonucleotides of the second RNA sequence (G) which are basepaired to ribonucleotides of the first RNA sequence (E),
wherein the one or more double-stranded product RNA molecule(s) (P) comprise the bulge, and
wherein ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P). Exemplary RNA molecules of this embodiment are illustrated schematically in FIG. 53, panels K, N and P.

In an embodiment of the second aspect, the first RNA sequence (E) is hybridised to the second RNA sequence (G) by basepairing between at least 21 of the at least 21 contiguous ribonucleotides of the first RNA sequence (E) and at least 21 of the at least 23 contiguous ribonucleotides of the second RNA sequence (G),
wherein 2 ribonucleotides of the at least 23 ribonucleotides of (G) are non-basepaired and all of the at least 21 ribonucleotides of (E) are basepaired in the part (C) of the double-stranded RNA region (B), the 2 non-basepaired ribonucleotide forming two single-ribonucleotide bulges in the part (C) of the double-stranded RNA region (B),
wherein each bulge is immediately flanked by ribonucleotides of the second RNA sequence (G) which are basepaired to ribonucleotides of the first RNA sequence (E),
wherein the one or more double-stranded product RNA molecule(s) (P) comprise the two bulges, and
wherein ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P). Exemplary RNA molecules of this embodiment are illustrated schematically in FIG. 53, panels L, M and Q.

In an embodiment of the second aspect, the first RNA sequence (E) is hybridised to the second RNA sequence (G) by basepairing between at least 20 of the at least 21 contiguous ribonucleotides of the first RNA sequence (E) and at least 20 of the at least 23 contiguous ribonucleotides of the second RNA sequence (G),
wherein 3 contiguous ribonucleotides of the at least 23 ribonucleotides of (G) are non-basepaired, and 1 ribonucleotide of the at least 21 ribonucleotides of (E) is non-basepaired in the part (C) of the double-stranded RNA region (B), the non-basepaired ribonucleotides forming a bulge in the part (C) of the double-stranded RNA region (B),
wherein the bulge is immediately flanked by ribonucleotides which are basepaired in the part (C) of the double-stranded RNA region (B),
wherein the one or more double-stranded product RNA molecule(s) (P) comprise the bulge, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P).

In an embodiment of the second aspect, the first RNA sequence (E) is hybridised to the second RNA sequence (G) by basepairing between at least 20 of the at least 21 contiguous ribonucleotides of the first RNA sequence (E) and at least 20 of the at least 23 contiguous ribonucleotides of the second RNA sequence (G),
wherein 3 ribonucleotides of the at least 23 ribonucleotides of (G) are non-basepaired, and 1 ribonucleotide of the at least 21 ribonucleotides of (E) is non-basepaired in the part (C) of the double-stranded RNA region (B), the non-basepaired ribonucleotides forming three bulges in the part (C) of the double-stranded RNA region (B),
wherein each bulge is immediately flanked by ribonucleotides which are basepaired in the part (C) of the double-stranded RNA region (B),
wherein one bulge is formed by a mismatched ribonucleotide pair between the first RNA sequence (E) and the second RNA sequence (G) and the other two bulges are single-ribonucleotide bulges,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P).

In an embodiment of the second aspect, the first RNA sequence (E) is hybridised to the second RNA sequence (G) by basepairing between at least 20 of the at least 21 contiguous ribonucleotides of the first RNA sequence (E) and at least 20 of the at least 23 contiguous ribonucleotides of the second RNA sequence (G),
wherein 3 ribonucleotides of the at least 23 ribonucleotides of (G) are non-basepaired, and 1 ribonucleotide of the at least 21 ribonucleotides of (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein one bulge is a single-ribonucleotide bulge, and the other non-basepaired ribonucleotides form the other bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P). Exemplary RNA molecules of this embodiment are illustrated schematically in FIG. 53, panel O.

In an embodiment of the second aspect, the double-stranded RNA region (B) comprises:
(a) a first RNA strand (D) which comprises a first RNA sequence (E) of at least 23 contiguous ribonucleotides, and
(b) a second RNA strand (F) which comprises a second RNA sequence (G) of at least 25 contiguous ribonucleotides,
wherein the first RNA strand (D) and second RNA strand (F) are covalently linked by a linking RNA sequence (L),
wherein the first RNA sequence (E) is hybridised to the second RNA sequence (G) by basepairing between at least 22 of the at least 23 contiguous ribonucleotides of the first RNA sequence (E) and at least 22 of the at least 25 contiguous ribonucleotides of the second RNA sequence (G), forming at least a part (C) of the double-stranded RNA region (B),
wherein 2 or 3 ribonucleotides of the at least 25 ribonucleotides of (G) are non-basepaired and 0 or 1, respectively, ribonucleotides of the at least 23 ribonucleotides of (E) are non-basepaired in the part (C) of the double-stranded RNA region (B), forming one, two, three or four bulges in the part (C) of the double-stranded RNA region (B),
wherein each of the bulges is immediately flanked by ribonucleotides which are basepaired in the part (C) of the double-stranded RNA region (B). Exemplary RNA molecules of this embodiment are illustrated schematically in FIG. 53, panels I to Q.

Thus, in an embodiment the present invention provides a precursor RNA molecule (A) comprising at least one double-stranded RNA region (B), wherein:
(i) the double-stranded RNA region (B) comprises:
   (a) a first RNA strand (D) which comprises a first RNA sequence (E) of at least 23 contiguous ribonucleotides, and
   (b) a second RNA strand (F) which comprises a second RNA sequence (G) of at least 25 contiguous ribonucleotides,
wherein the first RNA strand (D) and second RNA strand (F) are covalently linked by a linking RNA sequence (L),
wherein the first RNA sequence (E) is hybridised to the second RNA sequence (G) by basepairing between at least 22 of the at least 23 contiguous ribonucleotides of the first RNA sequence (E) and at least 22 of the at least 25 contiguous ribonucleotides of the second RNA sequence (G), forming at least a part (C) of the double-stranded RNA region (B),
wherein 2 or 3 ribonucleotides of the at least 25 ribonucleotides of (G) are non-basepaired and 0 or 1, respectively, ribonucleotides of the at least 23 ribonucleotides of (E) are non-basepaired in the part (C) of the double-stranded RNA region (B), forming one, two, three or four bulges in the part (C) of the double-stranded RNA region (B),
wherein each of the bulges is immediately flanked by ribonucleotides which are basepaired in the part (C) of the double-stranded RNA region (B); and
   (ii) the precursor RNA molecule (A) is capable of being cleaved in a eukaryotic cell by one or more ribonucleases (RNases) to produce one or more double-stranded product RNA molecule(s) (P) consisting of a sense RNA sequence (H) of 21 contiguous ribonucleotides from the first RNA sequence (E) and an antisense RNA sequence (J) of 23 contiguous ribonucleotides from the second RNA sequence (G),
wherein the one or more double-stranded product RNA molecule(s) (P) comprise the one, two, three or four bulges,
wherein 18 or 19 of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P),
wherein ribonucleotides 20 and 21 of the sense RNA sequence (H) form a 2-ribonucleotide unpaired 3′ overhang and ribonucleotides 22 and 23 of the antisense RNA sequence (J) form a 2-ribonucleotide unpaired 3′ overhang in the one or more double-stranded product RNA molecule(s) (P),
wherein ribonucleotides 1 and 2 of the sense RNA sequence (H) basepair with ribonucleotides 20 and 21, respectively, of the antisense RNA sequence (J) in the one or more double-stranded product RNA molecule(s) (P), and
wherein ribonucleotides 18 and 19 of the sense RNA sequence (H) basepair with ribonucleotides 1 and 2, respectively, of the antisense RNA sequence (J) in the one or more double-stranded product RNA molecule(s) (P).

In an embodiment of the second aspect, the first RNA sequence (E) is hybridised to the second RNA sequence (G) by basepairing between at least 22 of the at least 23 contiguous ribonucleotides of the first RNA sequence (E) and at least 22 of the at least 25 contiguous ribonucleotides of the second RNA sequence (G),
wherein 3 ribonucleotides, 2 of which are contiguous, of the at least 25 ribonucleotides of (G) are non-basepaired, and 1 ribonucleotide of the at least 23 ribonucleotides of (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein one bulge is formed by a mismatched ribonucleotide pair between the first RNA sequence (E) and the second RNA sequence (G) and the second bulge is a di-ribonucleotide bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P).

In an embodiment of the second aspect, the first RNA sequence (E) is hybridised to the second RNA sequence (G) by basepairing between at least 22 of the at least 23 contiguous ribonucleotides of the first RNA sequence (E) and at least 22 of the at least contiguous ribonucleotides of the second RNA sequence (G),
wherein 3 ribonucleotides of the at least 25 ribonucleotides of (G) are non-basepaired, and 1 ribonucleotide of the at least 23 ribonucleotides of (E) is non-basepaired in the part (C) of the double-stranded RNA region (B), the non-basepaired ribonucleotides forming four single-ribonucleotide bulges in the part (C) of the double-stranded RNA region (B),
wherein each bulge is immediately flanked by ribonucleotides which are basepaired in the part (C) of the double-stranded RNA region (B),
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P). Exemplary RNA molecules of this embodiment are illustrated schematically in FIG. 53, panel J.

In an embodiment of the second aspect, the first RNA sequence (E) is hybridised to the second RNA sequence (G) by basepairing between at least 23 of the at least 23 contiguous ribonucleotides of the first RNA sequence (E) and at least 23 of the at least 25 contiguous ribonucleotides of the second RNA sequence (G),
wherein 2 contiguous ribonucleotides of the at least 25 ribonucleotides of (G) are non-basepaired and all of the at least 23 ribonucleotides of (E) are basepaired in the part (C) of the double-stranded RNA region (B), the 2 non-basepaired ribonucleotides forming a di-ribonucleotide bulge in the part (C) of the double-stranded RNA region (B),
wherein the bulge is immediately flanked by ribonucleotides of the second RNA sequence (G) which are basepaired to ribonucleotides of the first RNA sequence (E),
wherein the one or more double-stranded product RNA molecule(s) (P) comprise the bulge, and
wherein ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P). Exemplary RNA molecules of this embodiment are illustrated schematically in FIG. 53, panels K and P.

In an embodiment of the second aspect, the first RNA sequence (E) is hybridised to the second RNA sequence (G) by basepairing between at least 23 of the at least 23 contiguous ribonucleotides of the first RNA sequence (E) and at least 23 of the at least contiguous ribonucleotides of the second RNA sequence (G),
wherein 2 ribonucleotides of the at least 25 ribonucleotides of (G) are non-basepaired and all of the at least 23 ribonucleotides of (E) are basepaired in the part (C) of the double-stranded RNA region (B), the 2 non-basepaired ribonucleotide forming two single-ribonucleotide bulges in the part (C) of the double-stranded RNA region (B),
wherein each bulge is immediately flanked by ribonucleotides of the second RNA sequence (G) which are basepaired to ribonucleotides of the first RNA sequence (E),
wherein the one or more double-stranded product RNA molecule(s) (P) comprise the two bulges, and
wherein ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P). Exemplary RNA molecules of this embodiment are illustrated schematically in FIG. 53, panels L, M and Q.

In an embodiment of the second aspect, the first RNA sequence (E) is hybridised to the second RNA sequence (G) by basepairing between at least 22 of the at least 23 contiguous ribonucleotides of the first RNA sequence (E) and at least 22 of the at least 25 contiguous ribonucleotides of the second RNA sequence (G),
wherein 3 contiguous ribonucleotides of the at least 25 ribonucleotides of (G) are non-basepaired, and 1 ribonucleotide of the at least 23 ribonucleotides of (E) is non-basepaired in the part (C) of the double-stranded RNA region (B), the non-basepaired ribonucleotides forming a bulge in the part (C) of the double-stranded RNA region (B),
wherein the bulge is immediately flanked by ribonucleotides which are basepaired in the part (C) of the double-stranded RNA region (B),
wherein the one or more double-stranded product RNA molecule(s) (P) comprise the bulge, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P).

In an embodiment of the second aspect, the first RNA sequence (E) is hybridised to the second RNA sequence (G) by basepairing between at least 22 of the at least 23 contiguous ribonucleotides of the first RNA sequence (E) and at least 22 of the at least 25 contiguous ribonucleotides of the second RNA sequence (G),
wherein 3 ribonucleotides of the at least 25 ribonucleotides of (G) are non-basepaired, and 1 ribonucleotide of the at least 23 ribonucleotides of (E) is non-basepaired in the part (C) of the double-stranded RNA region (B), the non-basepaired ribonucleotides forming three bulges in the part (C) of the double-stranded RNA region (B),
wherein each bulge is immediately flanked by ribonucleotides which are basepaired in the part (C) of the double-stranded RNA region (B),
wherein one bulge is formed by a mismatched ribonucleotide pair between the first RNA sequence (E) and the second RNA sequence (G) and the other two bulges are single-ribonucleotide bulges,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P).

In an embodiment of the second aspect, the first RNA sequence (E) is hybridised to the second RNA sequence (G) by basepairing between at least 22 of the at least 23 contiguous ribonucleotides of the first RNA sequence (E) and at least 22 of the at least 25 contiguous ribonucleotides of the second RNA sequence (G),
wherein 3 ribonucleotides of the at least 25 ribonucleotides of (G) are non-basepaired, and 1 ribonucleotide of the at least 23 ribonucleotides of (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein one bulge is a single-ribonucleotide bulge, and the other non-basepaired ribonucleotides form the other bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 21 of antisense RNA sequence (J) in the one or more double-stranded product RNA molecule(s) (P).

Each of the embodiments of the second aspect may have the following feature:
(i) ribonucleotides 1, 2 and 3 of the sense RNA sequence (H) basepair with ribonucleotides 19, 20 and 21, respectively, of the antisense RNA sequence (J) in at least some of the one or more double-stranded product RNA molecule(s) (P), or
(ii) ribonucleotides 17, 18 and 19 of the sense RNA sequence (H) basepair with ribonucleotides 1, 2 and 3, respectively, of the antisense RNA sequence (J) in at least some of the one or more double-stranded product RNA molecule(s) (P), or preferably both (i) and (ii). Exemplary RNA molecules of this embodiment are illustrated schematically in FIG. 53, panels I to Q, which all show product RNA molecules with at least three contiguous basepairs at both ends of the double-stranded region of the product RNA molecules. In further features of these embodiments, the product RNA molecules may have at least four, at least five or at least six contiguous basepairs at one or both ends of the double-stranded molecule before any bulge. In these embodiments, any of those basepairs may be G:U basepairs, preferably one or more of ribonucleotides 1, 2 and 3 of the antisense sequence (J) are involved in G:U basepairs, more preferably one or both of ribonucleotides 1 and 2 of the antisense sequence (J) are involved in G:U basepairs, even more preferably ribonucleotide 1 of the antisense sequence (J) is involved in G:U basepairs. Most preferably, one or more of ribonucleotides 1, 2 and 3 of the antisense sequence (J) are U ribonucleotides, or one or both of ribonucleotides 1 and 2 are U ribonucleotides, or ribonucleotide 1 is a U ribonucleotide, involved in G:U basepairs.

The design principles for the asymmetric precursor RNA molecules of the second aspect can be applied in an extended fashion to longer double-stranded regions. For example, the non-basepaired ribonucleotides in the second RNA sequence (G) (antisense sequence) may be arranged in a periodic fashion to provide a population of product RNA molecules (P) having multiple, non-overlapping antisense RNA sequences (J) of 23 nt. Such precursor RNA molecules are particularly useful for reducing expression of a target RNA molecule in a plant cell, fungal cell or nematode cell. They are also useful in other invertebrate animal cells such as an arthropod cell or insect cell, or in a non-mammalian vertebrate animal cell. They may be produced in a plant cell to reduce an insect target RNA molecule or a fungal pathogen or nematode target RNA molecule, or applied topically to a plant or insect to reduce a target RNA molecule. For example, in an embodiment of the second aspect, the first RNA sequence (E) comprises at least 44 contiguous ribonucleotides and the second RNA sequence (G) comprises at least 48 contiguous ribonucleotides,
wherein the first RNA sequence (E) is hybridised to the second RNA sequence (G) by basepairing between at least 42, preferably at least 43 or at least 44, of the at least 44 contiguous ribonucleotides of the first RNA sequence (E) and at least 42, preferably at least 43 or at least 44, of the at least 48 contiguous ribonucleotides of the second RNA sequence (G),
wherein 4, 5 or 6 ribonucleotides of the at least 48 contiguous ribonucleotides of (G) are non-basepaired, and 0, 1 or 2, respectively, ribonucleotides of the at least 44 contiguous ribonucleotides of (E) are non-basepaired in the part (C) of the double-stranded RNA region (B), forming bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 23 contiguous ribonucleotides,
wherein the multiple, different double-stranded product RNA molecule(s) (P) each comprise at least one of the bulges, and
wherein at least some of the multiple, different double-stranded product RNA molecules (P) comprise non-overlapping antisense RNA sequences (J), preferably adjacent non-overlapping antisense RNA sequences (J).

In another embodiment of the second aspect, the first RNA strand (D) comprises a first RNA sequence (E) of at least 44 contiguous ribonucleotides and the second RNA strand (F) comprises a second RNA sequence (G) of at least 48 contiguous wherein the first RNA sequence (E) is hybridised to the second RNA sequence (G) by basepairing between all 44 contiguous ribonucleotides of the first RNA sequence (E) and 44 of the 48 contiguous ribonucleotides of the second RNA sequence (G), forming a part (C) of the double-stranded RNA region (B),
wherein 4 ribonucleotides of the 48 contiguous ribonucleotides of (G) are non-basepaired in the part (C) of the double-stranded RNA region (B), forming 2, 3 or 4 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 23 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the bulges are preferably spaced apart by 6-16 contiguous basepairs, more preferably by 7-15 or 8-14 contiguous basepairs, even more preferably by 9-13 contiguous basepairs, most preferably by 10, 11 or 12 contiguous basepairs. In specific examples, the bulges are spaced apart by 6, 7, 8, 9, 10, 11, 12, 13, 14, 15 or 16 contiguous basepairs. In an embodiment, all of these basepairs are canonical basepairs. Alternatively, 1, 2, 3, 4 or 5 of the basepairs between the bulges are G:U basepairs. The precursor RNA molecule may comprise two or more such double-stranded RNA regions, for example comprise an ledRNA structure.

In a different embodiment with longer dsRNA regions of at least 44 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 44 contiguous ribonucleotides and the second RNA strand (F) comprises a second RNA sequence (G) of at least 48 contiguous ribonucleotides,
wherein the first RNA sequence (E) is hybridised to the second RNA sequence (G) by basepairing between all 44 contiguous ribonucleotides of the first RNA sequence (E) and 44 of the 48 contiguous ribonucleotides of the second RNA sequence (G), forming a part (C) of the double-stranded RNA region (B),
wherein 4 ribonucleotides of the 48 contiguous ribonucleotides of (G) are non-basepaired in the part (C) of the double-stranded RNA region (B), forming 2, 3 or 4 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 23 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the bulges are preferably spaced apart by 6-16 contiguous basepairs, more preferably by 7-15 or 8-14 contiguous basepairs, even more preferably by 9-13 contiguous basepairs, most preferably by 10, 11 or 12 contiguous basepairs. In specific examples, the bulges are spaced apart by 6, 7, 8, 9, 10, 11, 12, 13, 14, 15 or 16 contiguous basepairs. In a preferred example, all of these basepairs are canonical basepairs. Alternatively, 1, 2, 3, 4 or 5 of the basepairs between the bulges are G:U basepairs.

For example, in an embodiment, the first RNA sequence (E) comprises at least 65 contiguous ribonucleotides and the second RNA sequence (G) comprises at least 71 contiguous ribonucleotides, and wherein the first RNA sequence (E) is hybridised to the second RNA sequence (G) by basepairing between at least 62, preferably at least 63, at least 64 or at least 65, of the at least 65 contiguous ribonucleotides of the first RNA sequence (E) and at least 62, preferably at least 63, at least 64 or at least 65, of the at least 71 contiguous ribonucleotides of the second RNA sequence (G).

In another embodiment of the second aspect, the first RNA strand (D) comprises a first RNA sequence (E) of at least 65 contiguous ribonucleotides and the second RNA strand (F) comprises a second RNA sequence (G) of at least 71 contiguous
wherein the first RNA sequence (E) is hybridised to the second RNA sequence (G) by basepairing between all 65 contiguous ribonucleotides of the first RNA sequence (E) and 65 of the 71 contiguous ribonucleotides of the second RNA sequence (G), forming a part (C) of the double-stranded RNA region (B),
wherein 6 ribonucleotides of the 71 contiguous ribonucleotides of (G) are non-basepaired in the part (C) of the double-stranded RNA region (B), forming 3, 4, 5 or 6 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 23 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the bulges are preferably spaced apart periodically, for example by 6-16 contiguous basepairs, more preferably by 7-15 or 8-14 contiguous basepairs, even more preferably by 9-13 contiguous basepairs, most preferably by 10, 11 or 12 contiguous basepairs. In specific examples, the bulges are spaced apart by 6, 7, 8, 9, 10, 11, 12, 13, 14, 15 or 16 contiguous basepairs. In an example, all of these basepairs are canonical basepairs. Alternatively, 1, 2, 3, 4 or 5 of the basepairs between the bulges are G:U basepairs. The precursor RNA molecule may comprise two or more such double-stranded RNA regions, for example comprise an ledRNA structure.

In a different embodiment with longer dsRNA regions of at least 65 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 65 contiguous ribonucleotides and the second RNA strand (F) comprises a second RNA sequence (G) of at least 71 contiguous ribonucleotides,
wherein the first RNA sequence (E) is hybridised to the second RNA sequence (G) by basepairing between all 65 contiguous ribonucleotides of the first RNA sequence (E) and 65 of the 71 contiguous ribonucleotides of the second RNA sequence (G), forming a part (C) of the double-stranded RNA region (B),
wherein 6 ribonucleotides of the 71 contiguous ribonucleotides of (G) are non-basepaired in the part (C) of the double-stranded RNA region (B), forming 3, 4, 5 or 6 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 23 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the bulges are preferably spaced apart by 6-16 contiguous basepairs, more preferably by 7-15 or 8-14 contiguous basepairs, even more preferably by 9-13 contiguous basepairs, most preferably by 10, 11 or 12 contiguous basepairs. In specific examples, the bulges are spaced apart by 6, 7, 8, 9, 10, 11, 12, 13, 14, 15 or 16 contiguous basepairs. In a preferred example, all of these basepairs are canonical basepairs. Alternatively, 1, 2, 3, 4 or 5 of the basepairs between the bulges are G:U basepairs.

In the previous three embodiments, the first RNA sequence (E) and the second RNA sequence (G) may extend to longer than 65 and 71 ribonucleotides, respectively. In a further embodiment, the first RNA sequence (E) comprises at least 86 contiguous ribonucleotides and the second RNA sequence (G) comprises at least 94 contiguous ribonucleotides, and wherein the first RNA sequence (E) is hybridised to the second RNA sequence (G) by basepairing between at least 82, preferably at least 83, at least 84, at least 85 or at least 86, of the at least 86 contiguous ribonucleotides of the first RNA sequence (E) and at least 82, preferably at least 83, at least 84, at least 85 or at least 86, of the at least 94 contiguous ribonucleotides of the second RNA sequence (G).

In another embodiment of the second aspect, the first RNA strand (D) comprises a first RNA sequence (E) of at least 86 contiguous ribonucleotides and the second RNA strand (F) comprises a second RNA sequence (G) of at least 94 contiguous ribonucleotides,
wherein the first RNA sequence (E) is hybridised to the second RNA sequence (G) by basepairing between all 86 contiguous ribonucleotides of the first RNA sequence (E) and 86 of the 94 contiguous ribonucleotides of the second RNA sequence (G), forming a part (C) of the double-stranded RNA region (B),
wherein 8 ribonucleotides of the 94 contiguous ribonucleotides of (G) are non-basepaired in the part (C) of the double-stranded RNA region (B), forming 4-8 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 23 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the bulges are preferably spaced apart periodically, for example by 6-16 contiguous basepairs, more preferably by 7-15 or 8-14 contiguous basepairs, even more preferably by 9-13 contiguous basepairs, most preferably by 10, 11 or 12 contiguous basepairs. In specific examples, the bulges are spaced apart by 6, 7, 8, 9, 10, 11, 12, 13, 14, 15 or 16 contiguous basepairs. In a preferred example, all of these basepairs are canonical basepairs. Alternatively, 1, 2, 3, 4 or 5 of the basepairs between the bulges are G:U basepairs. The precursor RNA molecule may comprise two or more such double-stranded RNA regions, for example comprise a ledRNA structure.

In a different embodiment with longer dsRNA regions of at least 86 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 86 contiguous ribonucleotides and the second RNA strand (F) comprises a second RNA sequence (G) of at least 94 contiguous ribonucleotides,
wherein the first RNA sequence (E) is hybridised to the second RNA sequence (G) by basepairing between all 86 contiguous ribonucleotides of the first RNA sequence (E) and 86 of the 94 contiguous ribonucleotides of the second RNA sequence (G), forming a part (C) of the double-stranded RNA region (B),
wherein 8 ribonucleotides of the 94 contiguous ribonucleotides of (G) are non-basepaired in the part (C) of the double-stranded RNA region (B), forming 4-8 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 23 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the bulges are preferably spaced apart by 6-16 contiguous basepairs, more preferably by 7-15 or 8-14 contiguous basepairs, even more preferably by 9-13 contiguous basepairs, most preferably by 10, 11 or 12 contiguous basepairs. In specific examples, the bulges are spaced apart by 6, 7, 8, 9, 10, 11, 12, 13, 14, 15 or 16 contiguous basepairs. In a preferred example, all of these basepairs are canonical basepairs. Alternatively, 1, 2, 3, 4 or 5 of the basepairs between the bulges are G:U basepairs. In a preferred example, all of these basepairs are canonical basepairs. Alternatively, 1, 2, 3, 4 or 5 of the basepairs between the bulges are G:U basepairs.

In the previous three embodiments, the first RNA sequence (E) and the second RNA sequence (G) may extend to longer than 86 and 94 ribonucleotides, respectively. In a further embodiment of the second aspect, the first RNA sequence (E) comprises at least 107 contiguous ribonucleotides and the second RNA sequence (G) comprises at least 117 contiguous ribonucleotides, and wherein the first RNA sequence (E) is hybridised to the second RNA sequence (G) by basepairing between at least 102, preferably at least 103, at least 104, at least 105, at least 106 or at least 107, of the at least 107 contiguous ribonucleotides of the first RNA sequence (E) and at least 102, preferably at least 103, at least 104, at least 105, at least 106 or at least 107, of the at least 117 contiguous ribonucleotides of the second RNA sequence (G). In this embodiment, at least 10 ribonucleotides of the second RNA sequence, up to a maximum of 15 ribonucleotides, are non-basepaired and form bulges, preferably at least some are single ribonucleotide bulges.

In another embodiment of the second aspect, the first RNA strand (D) comprises a first RNA sequence (E) of at least 107 contiguous ribonucleotides and the second RNA strand (F) comprises a second RNA sequence (G) of at least 117 contiguous ribonucleotides,
wherein the first RNA sequence (E) is hybridised to the second RNA sequence (G) by basepairing between all 107 contiguous ribonucleotides of the first RNA sequence (E) and 107 of the 117 contiguous ribonucleotides of the second RNA sequence (G), forming a part (C) of the double-stranded RNA region (B),
wherein 10 ribonucleotides of the 117 contiguous ribonucleotides of (G) are non-basepaired in the part (C) of the double-stranded RNA region (B), forming 5-10 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 23 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the bulges are preferably spaced apart periodically, for example by 6-16 contiguous basepairs, more preferably by 7-15 or 8-14 contiguous basepairs, even more preferably by 9-13 contiguous basepairs, most preferably by 10, 11 or 12 contiguous basepairs. In specific examples, the bulges are spaced apart by 6, 7, 8, 9, 10, 11, 12, 13, 14, 15 or 16 contiguous basepairs. In an example, all of these basepairs are canonical basepairs. Alternatively, 1, 2, 3, 4 or 5 of the basepairs between the bulges are G:U basepairs. The precursor RNA molecule may comprise two or more such double-stranded RNA regions, for example comprise a ledRNA structure.

In a different embodiment with longer dsRNA regions of at least 107 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 107 contiguous ribonucleotides and the second RNA strand (F) comprises a second RNA sequence (G) of at least 117 contiguous ribonucleotides,
wherein the first RNA sequence (E) is hybridised to the second RNA sequence (G) by basepairing between all 107 contiguous ribonucleotides of the first RNA sequence (E) and 107 of the 117 contiguous ribonucleotides of the second RNA sequence (G), forming a part (C) of the double-stranded RNA region (B),
wherein 10 ribonucleotides of the 117 contiguous ribonucleotides of (G) are non-basepaired in the part (C) of the double-stranded RNA region (B), forming bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 23 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the bulges are preferably spaced apart by 6-16 contiguous basepairs, more preferably by 7-15 or 8-14 contiguous basepairs, even more preferably by 9-13 contiguous basepairs, most preferably by 10, 11 or 12 contiguous basepairs. In specific examples, the bulges are spaced apart by 6, 7, 8, 9, 10, 11, 12, 13, 14, 15 or 16 contiguous basepairs. In a preferred example, all of these basepairs are canonical basepairs. Alternatively, 1, 2, 3, 4 or 5 of the basepairs between the bulges are G:U basepairs.

In each of the above embodiments of the second aspect, the second RNA sequence (G) is longer than the first RNA sequence (E) because of the non-basepaired ribonucleotides that bulge out in (G). Preferably, the length of the first RNA sequence (E) is 90%-94% or 90%-93% or 91%-94% or 91%-93% of the length of the second RNA sequence (G). These features may also apply across the full length of the double-stranded region (B), where the part (C) of the double-stranded region is the full length of (B).

As the skilled person would appreciate, further embodiments include a first RNA sequence (E) and a second RNA sequence (G) having longer contiguous ribonucleotides following the same principles in the above embodiments.

In an embodiment of the first or second aspect, or the molecules lacking a loop, the first RNA sequence (E) and the second RNA sequence (G) both comprise at least 100 contiguous ribonucleotides, or at least 110 or 120, or at least 150, or at least 200, or at least 250, or at least 300 contiguous ribonucleotides, preferably to a maximum of 1000 ribonucleotides, more preferably to a maximum of 800 contiguous ribonucleotides, or even more preferably to a maximum of 600 contiguous ribonucleotides. For example, the first RNA sequence (E) and the second RNA sequence (G) both comprise contiguous ribonucleotides in the range 100-1000, 100-800, or 100-600 ribonucleotides, or in the range 150-1000, 150-800, or 150-600 ribonucleotides. In preferred embodiments, the length of the sense RNA sequence of the dsRNA region is 90%-97% or 90%-96% the length of the antisense sequence, for example 90%-94% or 90%-93% or 91%-94% or 91%-93% compared to the antisense sequence. These features are applicable to hairpin RNAs and to dsRNAs formed by annealing of two RNA strands i.e. without a joining loop sequence. Each of these features may also be applied to a second dsRNA region in the precursor RNA molecule, for example in a ledRNA molecule. In these embodiments, the eukaryotic cell in which the precursor RNA molecule (A) is cleaved is preferably a plant cell, a fungal cell, a nematode cell, or an arthropod cell such as an insect cell, or the target RNA molecule is preferably in a plant cell, a fungal cell, a nematode cell, or an arthropod cell such as an insect cell. Such precursor RNA molecules are particularly useful for reducing expression of a target RNA molecule in a plant cell, fungal cell or nematode cell. They are also useful in other invertebrate animal cells such as an arthropod cell, for example an insect cell, or in a non-mammalian vertebrate animal cell. They may be produced in a plant cell to reduce an insect target RNA molecule or a fungal pathogen or nematode target RNA molecule, or applied topically to a plant or insect to reduce a target RNA molecule.

Each of the embodiments of the second aspect, or the molecules lacking a loop, may have the following feature: the precursor RNA molecule is capable of being cleaved in a eukaryotic cell by one or more ribonucleases (RNases) to produce multiple, different double-stranded product RNA molecules (P) which each consist of a sense RNA sequence (H) of 21 contiguous ribonucleotides from the first RNA sequence (E) and an antisense RNA sequence (J) of 23 contiguous ribonucleotides from the second RNA sequence (G), wherein at least some of the multiple, different double-stranded product RNA molecules (P) have overlapping antisense RNA sequences (J). In a further embodiment, at least some of the multiple, different double-stranded product RNA molecules (P) have non-overlapping antisense RNA sequences (J), where the population of double-stranded product RNA molecules (P) produced from the precursor RNA molecule includes some overlapping and some non-overlapping antisense RNA sequences (J). Preferably, there are more non-overlapping antisense RNA sequences (J), as readily occurs with longer (>42 basepairs) double-stranded regions (B) where the siRNA molecules each consisting of 21 nt sense RNA sequences hybridised to 23 nt antisense RNA sequences are phased along the length of (B).

Each of the embodiments of the second aspect, or the molecules lacking a loop, may have the following feature: the precursor RNA molecule is capable of being cleaved in a eukaryotic cell by one or more ribonucleases (RNases) to produce multiple, different double-stranded product RNA molecules (P) which each consist of a sense RNA sequence (H) of 21 contiguous ribonucleotides from a first RNA sequence (E) and an antisense RNA sequence (J) of 23 contiguous ribonucleotides from a second RNA sequence (G), wherein at least some of the multiple, different double-stranded product RNA molecules (P) have non-overlapping antisense RNA sequences (J), preferably adjacent non-overlapping antisense RNA sequences (J).

In an embodiment of the second aspect, or the molecules lacking a loop, the molecule is capable of being cleaved in a eukaryotic cell by one or more ribonucleases (RNases) to produce multiple, different double-stranded product RNA molecules (P) which comprise double-stranded RNA product molecules as defined in two or more embodiments of the second aspect.

In a third aspect, the present invention provides asymmetric precursor RNA molecules which are processed to produce siRNA molecules consisting of 21 nt sense RNA sequences hybridised to 24 nt antisense RNA sequences. Therefore, in this aspect the present invention provides a precursor RNA molecule (A) comprising at least one double-stranded RNA region (B), wherein:
(i) the double-stranded RNA region (B) comprises:
   (a) a first RNA strand (D) of at least 23 contiguous ribonucleotides which comprises a first RNA sequence (E) of at least 21 contiguous ribonucleotides, and
   (b) a second RNA strand (F) of at least 26 contiguous ribonucleotides which comprises a second RNA sequence (G) of at least 24 contiguous
wherein the first RNA strand (D) and second RNA strand (F) are covalently linked by a linking RNA sequence (L),
wherein the first RNA sequence (E) is hybridised to the second RNA sequence (G) by basepairing between at least 20 of the at least 21 contiguous ribonucleotides of the first RNA sequence (E) and at least 20 of the at least 24 contiguous ribonucleotides of the second RNA sequence (G), forming at least a part (C) of the double-stranded RNA region (B),
wherein 3 or 4 ribonucleotides of the at least 24 ribonucleotides of (G) are non-basepaired and 0 or 1, respectively, ribonucleotides of the at least 21 ribonucleotides of (E) are non-basepaired in the part (C) of the double-stranded RNA region (B), forming one, two, three, four or five bulges in the part (C) of the double-stranded RNA region (B),
wherein each of the one, two, three, four or five bulges is immediately flanked by ribonucleotides which are basepaired in the part (C) of the double-stranded RNA region (B); and
   (ii) the precursor RNA molecule (A) is capable of being cleaved in a eukaryotic cell by one or more ribonucleases (RNases) to produce one or more double-stranded product RNA molecule(s) (P) consisting of a sense RNA sequence (H) of 21 contiguous ribonucleotides from the first RNA sequence (E) and an antisense RNA sequence (J) of 24 contiguous ribonucleotides from the second RNA strand (F), including at least 22 contiguous ribonucleotides from (G),
wherein the one or more double-stranded product RNA molecule(s) (P) comprise the one, two, three, four or five bulges,
wherein 18 or 19 of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P),
wherein ribonucleotides 20 and 21 of the sense RNA sequence (H) form a 2-ribonucleotide unpaired 3′ overhang and ribonucleotides 23 and 24 of the antisense RNA sequence (J) form a 2-ribonucleotide unpaired 3′ overhang in the one or more double-stranded product RNA molecule(s) (P),
wherein ribonucleotides 1 and 2 of the sense RNA sequence (H) basepair with ribonucleotides 21 and 22, respectively, of the antisense RNA sequence (J) in the one or more double-stranded product RNA molecule(s) (P), and
wherein ribonucleotides 18 and 19 of the sense RNA sequence (H) basepair with ribonucleotides 1 and 2, respectively, of the antisense RNA sequence (J) in the one or more double-stranded product RNA molecule(s) (P). Exemplary RNA molecules of this embodiment are illustrated schematically in FIG. 53, panels R to Z.

In an embodiment of the third aspect, one or both of ribonucleotides 1 and 2 of the at least 23 contiguous ribonucleotides of the first RNA strand (D) are not basepaired with one or both, respectively, of ribonucleotides 25 and 26 of the at least 26 contiguous ribonucleotides of the second RNA strand (F).

In an embodiment of the third aspect, the first RNA sequence (E) is hybridised to the second RNA sequence (G) by basepairing between at least 20 of the at least 21 contiguous ribonucleotides of the first RNA sequence (E) and at least 20 of the at least 24 contiguous ribonucleotides of the second RNA sequence (G),
wherein 4 ribonucleotides, 3 of which are contiguous, of the at least 24 ribonucleotides of (G) are non-basepaired and 1 ribonucleotide of the at least 21 ribonucleotides of (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein one bulge is formed by a mismatched ribonucleotide pair between the first RNA sequence (E) and the second RNA sequence (G) and the second bulge is a tri-ribonucleotide bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panel R.

In an embodiment of the third aspect, the first RNA sequence (E) is hybridised to the second RNA sequence (G) by basepairing between at least 20 of the at least 21 contiguous ribonucleotides of the first RNA sequence (E) and at least 20 of the at least 24 contiguous ribonucleotides of the second RNA sequence (G),
wherein 4 ribonucleotides of the at least 24 ribonucleotides of (G) are non-basepaired and 1 ribonucleotide of the at least 21 ribonucleotides of (E) is non-basepaired in the part (C) of the double-stranded RNA region (B), the non-basepaired ribonucleotides forming five single-ribonucleotide bulges in the part (C) of the double-stranded RNA region (B),
wherein each bulge is immediately flanked by ribonucleotides which are basepaired in the part (C) of the double-stranded RNA region (B),
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panel S.

In an embodiment of the third aspect, the first RNA sequence (E) is hybridised to the second RNA sequence (G) by basepairing between at least 21 of the at least 21 contiguous ribonucleotides of the first RNA sequence (E) and at least 21 of the at least 24 contiguous ribonucleotides of the second RNA sequence (G),
wherein 3 ribonucleotides of the at least 24 ribonucleotides of (G) are non-basepaired and all of the at least 21 ribonucleotides of (E) are basepaired in the part (C) of the double-stranded RNA region (B), the 3 non-basepaired ribonucleotides forming three single-ribonucleotide bulges in the part (C) of the double-stranded RNA region (B),
wherein each bulge is immediately flanked by ribonucleotides of the second RNA sequence (G) which are basepaired to ribonucleotides of the first RNA sequence (E),
wherein the one or more double-stranded product RNA molecule(s) (P) comprise the bulges, and
wherein ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panels T and Z.

In an embodiment of the third aspect, the first RNA sequence (E) is hybridised to the second RNA sequence (G) by basepairing between at least 21 of the at least 21 contiguous ribonucleotides of the first RNA sequence (E) and at least 21 of the at least 24 contiguous ribonucleotides of the second RNA sequence (G),
wherein 3 ribonucleotides of the at least 24 ribonucleotides of (G) are non-basepaired and all of the at least 21 ribonucleotides of (E) are basepaired in the part (C) of the double-stranded RNA region (B), the non-basepaired ribonucleotides forming two bulges in the part (C) of the double-stranded RNA region (B),
wherein each bulge is immediately flanked by ribonucleotides of the second RNA sequence (G) which are basepaired to ribonucleotides of the first RNA sequence (E),
wherein one bulge is a single-ribonucleotide bulge and the other bulge is a di-ribonucleotide bulge,
wherein the one or more double-stranded product RNA molecule(s) (P) comprise the bulges, and
wherein ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panels U and Y.

In an embodiment of the third aspect, the first RNA sequence (E) is hybridised to the second RNA sequence (G) by basepairing between at least 21 of the at least 21 contiguous ribonucleotides of the first RNA sequence (E) and at least 21 of the at least 24 contiguous ribonucleotides of the second RNA sequence (G),
wherein 3 contiguous ribonucleotides of the at least 24 ribonucleotides of (G) are non-basepaired and all of the at least 21 ribonucleotides of (E) are basepaired in the part (C) of the double-stranded RNA region (B), the 3 non-basepaired ribonucleotides forming a tri-ribonucleotide bulge in the part (C) of the double-stranded RNA region (B),
wherein the bulge is immediately flanked by ribonucleotides of the second RNA sequence (G) which are basepaired to ribonucleotides of the first RNA sequence (E),
wherein the one or more double-stranded product RNA molecule(s) (P) comprise the bulge, and
wherein ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panels V and X.

In an embodiment of the third aspect, the first RNA sequence (E) is hybridised to the second RNA sequence (G) by basepairing between at least 20 of the at least 21 contiguous ribonucleotides of the first RNA sequence (E) and at least 20 of the at least 24 contiguous ribonucleotides of the second RNA sequence (G),
wherein 4 contiguous ribonucleotides of the at least 24 ribonucleotides of (G) are non-basepaired and 1 ribonucleotide of the at least 21 ribonucleotides of (E) is non-basepaired in the part (C) of the double-stranded RNA region (B), the non-basepaired ribonucleotides forming a bulge in the part (C) of the double-stranded RNA region (B),
wherein the bulge is immediately flanked by ribonucleotides which are basepaired in the part (C) of the double-stranded RNA region (B),
wherein the one or more double-stranded product RNA molecule(s) (P) comprise the bulge, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P).

In an embodiment of the third aspect, the first RNA sequence (E) is hybridised to the second RNA sequence (G) by basepairing between at least 20 of the at least 21 contiguous ribonucleotides of the first RNA sequence (E) and at least 20 of the at least 24 contiguous ribonucleotides of the second RNA sequence (G),
wherein 4 ribonucleotides of the at least 24 ribonucleotides of (G) are non-basepaired and 1 ribonucleotide of the at least 21 ribonucleotides of (E) is non-basepaired in the part (C) of the double-stranded RNA region (B), the non-basepaired ribonucleotides forming four bulges in the part (C) of the double-stranded RNA region (B),
wherein each bulge is immediately flanked by ribonucleotides which are basepaired in the part (C) of the double-stranded RNA region (B),
wherein one bulge is formed by a mismatched ribonucleotide pair between the first RNA sequence (E) and the second RNA sequence (G), and other three bulges are single nucleotide bulges,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P).

In an embodiment of the third aspect, the first RNA sequence (E) is hybridised to the second RNA sequence (G) by basepairing between at least 20 of the at least 21 contiguous ribonucleotides of the first RNA sequence (E) and at least 20 of the at least 24 contiguous ribonucleotides of the second RNA sequence (G),
wherein 4 ribonucleotides, 2 of which are contiguous, of the at least 24 ribonucleotides of (G) are non-basepaired and 1 ribonucleotide of the at least 21 ribonucleotides of (E) is non-basepaired in the part (C) of the double-stranded RNA region (B), the non-basepaired ribonucleotides forming three bulges in the part (C) of the double-stranded RNA region (B),
wherein each bulge is immediately flanked by ribonucleotides which are basepaired in the part (C) of the double-stranded RNA region (B),
wherein two of the bulges are single nucleotide bulges, and the other non-basepaired ribonucleotides form the other bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P).

In an embodiment of the third aspect, the first RNA sequence (E) is hybridised to the second RNA sequence (G) by basepairing between at least 20 of the at least 21 contiguous ribonucleotides of the first RNA sequence (E) and at least 20 of the at least 24 contiguous ribonucleotides of the second RNA sequence (G),
wherein 4 ribonucleotides, 2 of which are contiguous, of the at least 24 ribonucleotides of (G) are non-basepaired and 1 ribonucleotide of the at least 21 ribonucleotides of (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein one bulge is a di-ribonucleotide bulge and the other non-basepaired ribonucleotides form the other bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P).

In an embodiment of the third aspect, the double-stranded RNA region (B) comprises:
(a) a first RNA strand (D) which comprises a first RNA sequence (E) of at least 23 contiguous ribonucleotides, and
(b) a second RNA strand (F) which comprises a second RNA sequence (G) of at least 26 contiguous ribonucleotides,
wherein the first RNA strand (D) and second RNA strand (F) are covalently linked by a linking RNA sequence (L),
wherein the first RNA sequence (E) is hybridised to the second RNA sequence (G) by basepairing between at least 22 of the at least 23 contiguous ribonucleotides of the first RNA sequence (E) and at least 22 of the at least 26 contiguous ribonucleotides of the second RNA sequence (G), forming at least a part (C) of the double-stranded RNA region (B),
wherein 3 or 4 ribonucleotides of the at least 26 ribonucleotides of (G) are non-basepaired and 0 or 1, respectively, ribonucleotides of the at least 23 ribonucleotides of (E) are non-basepaired in the part (C) of the double-stranded RNA region (B), forming one, two, three, four or five bulges in the part (C) of the double-stranded RNA region (B),
wherein each of the bulges is immediately flanked by ribonucleotides which are basepaired in the part (C) of the double-stranded RNA region (B). Exemplary RNA molecules of this embodiment are illustrated schematically in FIG. 53, panels R to V.

Thus, in an embodiment the present invention provides a precursor RNA molecule (A) comprising at least one double-stranded RNA region (B), wherein:
(i) the double-stranded RNA region (B) comprises:
   (a) a first RNA strand (D) which comprises a first RNA sequence (E) of at least 23 contiguous ribonucleotides, and
   (b) a second RNA strand (F) which comprises a second RNA sequence (G) of at least 26 contiguous ribonucleotides,
wherein the first RNA strand (D) and second RNA strand (F) are covalently linked by a linking RNA sequence (L),
wherein the first RNA sequence (E) is hybridised to the second RNA sequence (G) by basepairing between at least 22 of the at least 23 contiguous ribonucleotides of the first RNA sequence (E) and at least 22 of the at least 26 contiguous ribonucleotides of the second RNA sequence (G), forming at least a part (C) of the double-stranded RNA region (B),
wherein 3 or 4 ribonucleotides of the at least 26 ribonucleotides of (G) are non-basepaired and 0 or 1, respectively, ribonucleotides of the at least 23 ribonucleotides of (E) are non-basepaired in the part (C) of the double-stranded RNA region (B), forming one, two, three, four or five bulges in the part (C) of the double-stranded RNA region (B),
wherein each of the bulges is immediately flanked by ribonucleotides which are basepaired in the part (C) of the double-stranded RNA region (B); and
   (ii) the precursor RNA molecule (A) is capable of being cleaved in a eukaryotic cell by one or more ribonucleases (RNases) to produce one or more double-stranded product RNA molecule(s) (P) consisting of a sense RNA sequence (H) of 21 contiguous ribonucleotides from the first RNA sequence (E) and an antisense RNA sequence (J) of 24 contiguous ribonucleotides from the second RNA sequence (G),
wherein the one or more double-stranded product RNA molecule(s) (P) comprise the one, two, three, four or five bulges,
wherein 18 or 19 of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P),
wherein ribonucleotides 20 and 21 of the sense RNA sequence (H) form a 2-ribonucleotide unpaired 3′ overhang and ribonucleotides 23 and 24 of the antisense RNA sequence (J) form a 2-ribonucleotide unpaired 3′ overhang in the one or more double-stranded product RNA molecule(s) (P),
wherein ribonucleotides 1 and 2 of the sense RNA sequence (H) basepair with ribonucleotides 21 and 22, respectively, of the antisense RNA sequence (J) in the one or more double-stranded product RNA molecule(s) (P), and
wherein ribonucleotides 18 and 19 of the sense RNA sequence (H) basepair with ribonucleotides 1 and 2, respectively, of the antisense RNA sequence (J) in the one or more double-stranded product RNA molecule(s) (P). Exemplary RNA molecules of this embodiment are illustrated schematically in FIG. 53, panels R to V.

In an embodiment of the third aspect, the first RNA sequence (E) is hybridised to the second RNA sequence (G) by basepairing between at least 22 of the at least 23 contiguous ribonucleotides of the first RNA sequence (E) and at least 22 of the at least 26 contiguous ribonucleotides of the second RNA sequence (G),
wherein 4 ribonucleotides, 3 of which are contiguous, of the at least 26 ribonucleotides of (G) are non-basepaired and 1 ribonucleotide of the at least 23 ribonucleotides of (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein one bulge is formed by a mismatched ribonucleotide pair between the first RNA sequence (E) and the second RNA sequence (G) and the second bulge is a tri-ribonucleotide bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panel R.

In an embodiment of the third aspect, the first RNA sequence (E) is hybridised to the second RNA sequence (G) by basepairing between at least 22 of the at least 23 contiguous ribonucleotides of the first RNA sequence (E) and at least 22 of the at least 26 contiguous ribonucleotides of the second RNA sequence (G),
wherein 4 ribonucleotides of the at least 26 ribonucleotides of (G) are non-basepaired and 1 ribonucleotide of the at least 23 ribonucleotides of (E) is non-basepaired in the part (C) of the double-stranded RNA region (B), the non-basepaired ribonucleotides forming five single-ribonucleotide bulges in the part (C) of the double-stranded RNA region (B),
wherein each bulge is immediately flanked by ribonucleotides which are basepaired in the part (C) of the double-stranded RNA region (B),
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panel S.

In an embodiment of the third aspect, the first RNA sequence (E) is hybridised to the second RNA sequence (G) by basepairing between at least 23 of the at least 23 contiguous ribonucleotides of the first RNA sequence (E) and at least 23 of the at least 26 contiguous ribonucleotides of the second RNA sequence (G),
wherein 3 ribonucleotides of the at least 26 ribonucleotides of (G) are non-basepaired and all of the at least 23 ribonucleotides of (E) are basepaired in the part (C) of the double-stranded RNA region (B), the 3 non-basepaired ribonucleotides forming three single-ribonucleotide bulges in the part (C) of the double-stranded RNA region (B),
wherein each bulge is immediately flanked by ribonucleotides of the second RNA sequence (G) which are basepaired to ribonucleotides of the first RNA sequence (E),
wherein the one or more double-stranded product RNA molecule(s) (P) comprise the bulges, and
wherein ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panels T and Z.

In an embodiment of the third aspect, the first RNA sequence (E) is hybridised to the second RNA sequence (G) by basepairing between at least 23 of the at least 23 contiguous ribonucleotides of the first RNA sequence (E) and at least 23 of the at least 26 contiguous ribonucleotides of the second RNA sequence (G),
wherein 3 ribonucleotides of the at least 26 ribonucleotides of (G) are non-basepaired and all of the at least 23 ribonucleotides of (E) are basepaired in the part (C) of the double-stranded RNA region (B), the non-basepaired ribonucleotides forming two bulges in the part (C) of the double-stranded RNA region (B),
wherein each bulge is immediately flanked by ribonucleotides of the second RNA sequence (G) which are basepaired to ribonucleotides of the first RNA sequence (E),
wherein one bulge is a single-ribonucleotide bulge, and the other bulge is a di-ribonucleotide bulge,
wherein the one or more double-stranded product RNA molecule(s) (P) comprise the bulges, and
wherein ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panels U and Y.

In an embodiment of the third aspect, the first RNA sequence (E) is hybridised to the second RNA sequence (G) by basepairing between at least 23 of the at least 23 contiguous ribonucleotides of the first RNA sequence (E) and at least 23 of the at least 26 contiguous ribonucleotides of the second RNA sequence (G),
wherein 3 contiguous ribonucleotides of the at least 26 ribonucleotides of (G) are non-basepaired and all of the at least 23 ribonucleotides of (E) are basepaired in the part (C) of the double-stranded RNA region (B), the 3 non-basepaired ribonucleotides forming a tri-ribonucleotide bulge in the part (C) of the double-stranded RNA region (B),
wherein the bulge is immediately flanked by ribonucleotides of the second RNA sequence (G) which are basepaired to ribonucleotides of the first RNA sequence (E),
wherein the one or more double-stranded product RNA molecule(s) (P) comprise the bulge, and
wherein ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P). Exemplary RNA molecules of this embodiment are illustrated schematically in FIG. 53, panels V and X.

In an embodiment of the third aspect, the first RNA sequence (E) is hybridised to the second RNA sequence (G) by basepairing between at least 22 of the at least 23 contiguous ribonucleotides of the first RNA sequence (E) and at least 22 of the at least 26 contiguous ribonucleotides of the second RNA sequence (G),
wherein 4 contiguous ribonucleotides of the at least 26 ribonucleotides of (G) are non-basepaired and 1 ribonucleotide of the at least 23 ribonucleotides of (E) is non-basepaired in the part (C) of the double-stranded RNA region (B), the non-basepaired ribonucleotides forming a bulge in the part (C) of the double-stranded RNA region (B),
wherein the bulge is immediately flanked by ribonucleotides which are basepaired in the part (C) of the double-stranded RNA region (B),
wherein the one or more double-stranded product RNA molecule(s) (P) comprise the bulge, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P).

In an embodiment of the third aspect, the first RNA sequence (E) is hybridised to the second RNA sequence (G) by basepairing between at least 22 of the at least 23 contiguous ribonucleotides of the first RNA sequence (E) and at least 22 of the at least 26 contiguous ribonucleotides of the second RNA sequence (G),
wherein 4 ribonucleotides of the at least 26 ribonucleotides of (G) are non-basepaired and 1 ribonucleotide of the at least 23 ribonucleotides of (E) is non-basepaired in the part (C) of the double-stranded RNA region (B), the non-basepaired ribonucleotides forming four bulges in the part (C) of the double-stranded RNA region (B),
wherein each bulge is immediately flanked by ribonucleotides which are basepaired in the part (C) of the double-stranded RNA region (B),
wherein one bulge is formed by a mismatched ribonucleotide pair between the first RNA sequence (E) and the second RNA sequence (G), and other three bulges are single nucleotide bulges,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P).

In an embodiment of the third aspect, the first RNA sequence (E) is hybridised to the second RNA sequence (G) by basepairing between at least 22 of the at least 23 contiguous ribonucleotides of the first RNA sequence (E) and at least 22 of the at least 26 contiguous ribonucleotides of the second RNA sequence (G),
wherein 4 ribonucleotides, 2 of which are contiguous, of the at least 26 ribonucleotides of (G) are non-basepaired and 1 ribonucleotide of the at least 23 ribonucleotides of (E) is non-basepaired in the part (C) of the double-stranded RNA region (B), the non-basepaired ribonucleotides forming three bulges in the part (C) of the double-stranded RNA region (B),
wherein each bulge is immediately flanked by ribonucleotides which are basepaired in the part (C) of the double-stranded RNA region (B),
wherein two of the bulges are single nucleotide bulges and the other non-basepaired ribonucleotides form the other bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P).

In an embodiment of the third aspect, the first RNA sequence (E) is hybridised to the second RNA sequence (G) by basepairing between at least 22 of the at least 23 contiguous ribonucleotides of the first RNA sequence (E) and at least 22 of the at least 26 contiguous ribonucleotides of the second RNA sequence (G),
wherein 4 ribonucleotides, 2 of which are contiguous, of the at least 26 ribonucleotides of (G) are non-basepaired and 1 ribonucleotide of the at least 23 ribonucleotides of (E) is non-basepaired in the part (C) of the double-stranded RNA region (B), the non-basepaired ribonucleotides forming two bulges in the part (C) of the double-stranded RNA region (B),
wherein each bulge is immediately flanked by ribonucleotides which are basepaired in the part (C) of the double-stranded RNA region (B),
wherein one bulge is a di-nucleotide bulge and the other non-basepaired ribonucleotides form the other bulge,
wherein the one or more double-stranded product RNA molecule(s) (P) comprise the bulges, and
wherein all but one of ribonucleotides 1 to 19 of the sense RNA sequence (H) each basepair with a ribonucleotide of ribonucleotides 1 to 22 of antisense RNA sequence (J) in the one or more double-stranded product RNA molecule(s) (P).

Each of the embodiments of the third aspect may have the following feature:
(i) ribonucleotides 1, 2 and 3 of the sense RNA sequence (H) basepair with ribonucleotides 20, 21 and 22, respectively, of the antisense RNA sequence (J) in at least some of the one or more double-stranded product RNA molecule(s) (P), or
(ii) ribonucleotides 17, 18 and 19 of the sense RNA sequence (H) basepair with ribonucleotides 1, 2 and 3, respectively, of the antisense RNA sequence (J) in at least some of the one or more double-stranded product RNA molecule(s) (P), or preferably both (i) and (ii). Exemplary RNA molecules of this embodiment are illustrated schematically in FIG. 53, panels R to Z, which all show product RNA molecules with at least three contiguous basepairs at both ends of the product RNA molecules. In further features of these embodiments, the product RNA molecules may have at least four, at least five or at least six contiguous basepairs at one or both ends of the double-stranded molecule before any bulge. In these embodiments, any of those basepairs may be G:U basepairs, preferably one or more of ribonucleotides 1, 2 and 3 of the antisense sequence (J) are involved in G:U basepairs, more preferably one or both of ribonucleotides 1 and 2 of the antisense sequence (J) are involved in G:U basepairs, even more preferably ribonucleotide 1 of the antisense sequence (J) is involved in G:U basepairs. Most preferably, one or more of ribonucleotides 1, 2 and 3 of the antisense sequence (J) are U ribonucleotides, or one or both of ribonucleotides 1 and 2 are U ribonucleotides, or ribonucleotide 1 is a U ribonucleotide, involved in G:U basepairs.

The design principles for the asymmetric precursor RNA molecules of the third aspect can be applied in an extended fashion to longer double-stranded regions. For example, the non-basepaired ribonucleotides in the second RNA sequence (G) (antisense sequence) may be arranged in a periodic fashion to provide a population of product RNA molecules (P) having multiple, non-overlapping antisense RNA sequences (J) of 24 nt. Such precursor RNA molecules are particularly useful for reducing expression of a target RNA molecule in a plant cell, fungal cell or nematode cell. They are also useful in other invertebrate animal cells such as an arthropod cell or insect cell, or in a non-mammalian vertebrate animal cell. They may be produced in a plant cell to reduce an insect target RNA molecule or a fungal pathogen or nematode target RNA molecule, or applied topically to a plant or insect to reduce a target RNA molecule. For example, in an embodiment of the third aspect, the first RNA sequence (E) comprises at least 44 contiguous ribonucleotides and the second RNA sequence (G) comprises at least 50 contiguous ribonucleotides,
wherein the first RNA sequence (E) is hybridised to the second RNA sequence (G) by basepairing between at least 42, preferably at least 43 or at least 44, of the at least 44 contiguous ribonucleotides of the first RNA sequence (E) and at least 42, preferably at least 43 or at least 44, of the at least 50 contiguous ribonucleotides of the second RNA sequence (G),
wherein 6, 7 or 8 ribonucleotides of the at least 50 contiguous ribonucleotides of (G) are non-basepaired, and 0, 1 or 2, respectively, ribonucleotides of the at least 44 contiguous ribonucleotides of (E) are non-basepaired in the part (C) of the double-stranded RNA region (B), forming bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 24 contiguous ribonucleotides,
wherein the multiple, different double-stranded product RNA molecule(s) (P) each comprise at least one of the bulges, and
wherein at least some of the multiple, different double-stranded product RNA molecules (P) comprise non-overlapping antisense RNA sequences (J), preferably adjacent non-overlapping antisense RNA sequences (J).

In another embodiment of the third aspect, the first RNA strand (D) comprises a first RNA sequence (E) of at least 44 contiguous ribonucleotides and the second RNA strand (F) comprises a second RNA sequence (G) of at least 50 contiguous ribonucleotides,
wherein the first RNA sequence (E) is hybridised to the second RNA sequence (G) by basepairing between all 44 contiguous ribonucleotides of the first RNA sequence (E) and 44 of the 50 contiguous ribonucleotides of the second RNA sequence (G), forming a part (C) of the double-stranded RNA region (B),
wherein 6 ribonucleotides of the 50 contiguous ribonucleotides of (G) are non-basepaired in the part (C) of the double-stranded RNA region (B), forming 2-6 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 24 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the bulges are preferably spaced apart by 6-23 contiguous basepairs, more preferably by 7-22 or 8-21 contiguous basepairs, even more preferably by 9-20 contiguous basepairs, most preferably by 7, 8, 9, 10, 11 or 12 contiguous basepairs, or 7-12 or 7-11 or 7-10 contiguous basepairs. In specific examples, the bulges are spaced apart by 6, 7, 8, 9, 10, 11, 12, 13, 14, 15, 16, 17, 18, 19, 20 or 21 contiguous basepairs. In an embodiment, all of these basepairs are canonical basepairs. Alternatively, 1, 2, 3, 4, 5 or 6 of the basepairs between the bulges are G:U basepairs. The precursor RNA molecule may comprise two or more such double-stranded RNA regions, for example comprise a ledRNA structure.

In a different embodiment with longer dsRNA regions of at least 44 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 44 contiguous ribonucleotides and the second RNA strand (F) comprises a second RNA sequence (G) of at least 50 contiguous ribonucleotides,
wherein the first RNA sequence (E) is hybridised to the second RNA sequence (G) by basepairing between all 44 contiguous ribonucleotides of the first RNA sequence (E) and 44 of the 50 contiguous ribonucleotides of the second RNA sequence (G), forming a part (C) of the double-stranded RNA region (B),
wherein 6 ribonucleotides of the 50 contiguous ribonucleotides of (G) are non-basepaired in the part (C) of the double-stranded RNA region (B), forming 2-6 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 24 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the bulges are preferably spaced apart by 6-23 contiguous basepairs, more preferably by 7-22 or 8-21 contiguous basepairs, even more preferably by 9-20 contiguous basepairs, most preferably by 7, 8, 9, 10, 11 or 12 contiguous basepairs, or 7-12 or 7-11 or 7-10 contiguous basepairs. In specific examples, the bulges are spaced apart by 6, 7, 8, 9, 10, 11, 12, 13, 14, 15, 16, 17, 18, 19, 20 or 21 contiguous basepairs. In a preferred example, all of these basepairs are canonical basepairs. Alternatively, 1, 2, 3, 4, 5 or 6 of the basepairs between the bulges are G:U basepairs.

In an embodiment of the third aspect, the first RNA sequence (E) comprises at least 65 contiguous ribonucleotides and the second RNA sequence (G) comprises at least 74 contiguous ribonucleotides,
wherein the first RNA sequence (E) is hybridised to the second RNA sequence (G) by basepairing between at least 62, preferably at least 63 or at least 64 or at least 65, of the at least 65 contiguous ribonucleotides of the first RNA sequence (E) and at least 62, preferably at least 63 or at least 64 or at least 65, of the at least 74 contiguous ribonucleotides of the second RNA sequence (G),
wherein 9-12 ribonucleotides of the at least 74 contiguous ribonucleotides of (G) are non-basepaired, and 0-3 ribonucleotides of the at least 44 contiguous ribonucleotides of (E) are non-basepaired in the part (C) of the double-stranded RNA region (B), forming bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 24 contiguous ribonucleotides,
wherein the multiple, different double-stranded product RNA molecule(s) (P) each comprise at least one of the bulges, and
wherein at least some of the multiple, different double-stranded product RNA molecules (P) comprise non-overlapping antisense RNA sequences (J), preferably adjacent non-overlapping antisense RNA sequences (J).

In another embodiment of the third aspect, the first RNA strand (D) comprises a first RNA sequence (E) of at least 65 contiguous ribonucleotides and the second RNA strand (F) comprises a second RNA sequence (G) of at least 74 contiguous ribonucleotides,
wherein the first RNA sequence (E) is hybridised to the second RNA sequence (G) by basepairing between all 65 contiguous ribonucleotides of the first RNA sequence (E) and 65 of the 74 contiguous ribonucleotides of the second RNA sequence (G), forming a part (C) of the double-stranded RNA region (B),
wherein 9 ribonucleotides of the 74 contiguous ribonucleotides of (G) are non-basepaired in the part (C) of the double-stranded RNA region (B), forming 3-9 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 24 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the bulges are preferably spaced apart by 6-23 contiguous basepairs, more preferably by 7-22 or 8-21 contiguous basepairs, even more preferably by 9-20 contiguous basepairs, most preferably by 7, 8, 9, 10, 11 or 12 contiguous basepairs, or 7-12 or 7-11 or 7-10 contiguous basepairs. In specific examples, the bulges are spaced apart by 6, 7, 8, 9, 10, 11, 12, 13, 14, 15, 16, 17, 18, 19, 20 or 21 contiguous basepairs. In an embodiment, all of these basepairs are canonical basepairs. Alternatively, 1, 2, 3, 4, 5 or 6 of the basepairs between the bulges are G:U basepairs. The precursor RNA molecule may comprise two or more such double-stranded RNA regions, for example comprise an ledRNA structure.

In a different embodiment with longer dsRNA regions of at least 65 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 65 contiguous ribonucleotides and the second RNA strand (F) comprises a second RNA sequence (G) of at least 74 contiguous ribonucleotides,
wherein the first RNA sequence (E) is hybridised to the second RNA sequence (G) by basepairing between all 65 contiguous ribonucleotides of the first RNA sequence (E) and 65 of the 74 contiguous ribonucleotides of the second RNA sequence (G), forming a part (C) of the double-stranded RNA region (B),
wherein 9 ribonucleotides of the 74 contiguous ribonucleotides of (G) are non-basepaired in the part (C) of the double-stranded RNA region (B), forming 3-9 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 24 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the bulges are preferably spaced apart by 6-23 contiguous basepairs, more preferably by 7-22 or 8-21 contiguous basepairs, even more preferably by 9-20 contiguous basepairs, most preferably by 7, 8, 9, 10, 11 or 12 contiguous basepairs, or 7-12 or 7-11 or 7-10 contiguous basepairs. In specific examples, the bulges are spaced apart by 6, 7, 8, 9, 10, 11, 12, 13, 14, 15, 16, 17, 18, 19, 20 or 21 contiguous basepairs. In a preferred example, all of these basepairs are canonical basepairs. Alternatively, 1, 2, 3, 4, 5 or 6 of the basepairs between the bulges are G:U basepairs.

In the previous three embodiments, the first RNA sequence (E) and the second RNA sequence (G) may extend to longer than 65 and 74 ribonucleotides, respectively. In a further embodiment, the first RNA sequence (E) comprises at least 86 contiguous ribonucleotides and the second RNA sequence (G) comprises at least 98 contiguous ribonucleotides, and wherein the first RNA sequence (E) is hybridised to the second RNA sequence (G) by basepairing between at least 82, preferably at least 83, at least 84, at least 85 or at least 86, of the at least 86 contiguous ribonucleotides of the first RNA sequence (E) and at least 82, preferably at least 83, at least 84, at least 85 or at least 86, of the at least 98 contiguous ribonucleotides of the second RNA sequence (G).

In another embodiment of the third aspect, the first RNA strand (D) comprises a first RNA sequence (E) of at least 86 contiguous ribonucleotides and the second RNA strand (F) comprises a second RNA sequence (G) of at least 98 contiguous ribonucleotides,
wherein the first RNA sequence (E) is hybridised to the second RNA sequence (G) by basepairing between all 86 contiguous ribonucleotides of the first RNA sequence (E) and 86 of the 98 contiguous ribonucleotides of the second RNA sequence (G), forming a part (C) of the double-stranded RNA region (B),
wherein 12 ribonucleotides of the 98 contiguous ribonucleotides of (G) are non-basepaired in the part (C) of the double-stranded RNA region (B), forming 4-12 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 24 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this and the previous embodiments, the bulges are preferably spaced apart periodically, for example by 6-23 contiguous basepairs, more preferably by 7-−2 or 8-21 contiguous basepairs, even more preferably by 9-20 contiguous basepairs, most preferably by 7, 8, 9, 10, 11 or 12 contiguous basepairs, or 7-12 or 7-11 or 7-10 contiguous basepairs. In specific examples, the bulges are spaced apart by 6, 7, 8, 9, 10, 11, 12, 13, 14, 15, 16, 17, 18, 19, 20 or 21 contiguous basepairs. In a preferred example, all of these basepairs are canonical basepairs. Alternatively, 1, 2, 3, 4, 5 or 6 of the basepairs between the bulges are G:U basepairs. The precursor RNA molecule may comprise two or more such double-stranded RNA regions, for example comprise a ledRNA structure.

In a different embodiment with longer dsRNA regions of at least 86 basepairs, the precursor RNA molecule lacks the linking RNA sequence (L) and the first RNA strand (D) and the second RNA strand (F) hybridise to form the double-stranded RNA region (B). Such precursor RNA molecule may be readily produced in a cell-free system, for example in vitro. In an embodiment, the first RNA strand (D) comprises a first RNA sequence (E) of at least 86 contiguous ribonucleotides and the second RNA strand (F) comprises a second RNA sequence (G) of at least 98 contiguous ribonucleotides,
wherein the first RNA sequence (E) is hybridised to the second RNA sequence (G) by basepairing between all 86 contiguous ribonucleotides of the first RNA sequence (E) and 86 of the 98 contiguous ribonucleotides of the second RNA sequence (G), forming a part (C) of the double-stranded RNA region (B),
wherein 12 ribonucleotides of the 98 contiguous ribonucleotides of (G) are non-basepaired in the part (C) of the double-stranded RNA region (B), forming 4-12 bulges in the part (C) of the double-stranded RNA region (B),
wherein each of the bulges is immediately flanked by ribonucleotides which are basepaired in the part (C) of the double-stranded RNA region (B),
wherein the precursor RNA molecule is capable of being cleaved in a eukaryotic cell by one or more ribonucleases (RNases) to produce multiple, different double-stranded product RNA molecules (P) consisting of a sense RNA sequence (H) of 21 contiguous ribonucleotides and an antisense RNA sequence (J) of 24 contiguous ribonucleotides,
wherein the multiple, different double-stranded product RNA molecule(s) (P) each comprise at least one of the bulges, and
wherein at least some of the multiple, different double-stranded product RNA molecules (P) have non-overlapping antisense RNA sequences (J), preferably adjacent non-overlapping antisense RNA sequences (J). In this embodiment, the bulges are preferably spaced apart by 66-23 contiguous basepairs, more preferably by 7-22 or 8-21 contiguous basepairs, even more preferably by 9-20 contiguous basepairs, most preferably by 7, 8, 9, 10, 11 or 12 contiguous basepairs, or 7-12 or 7-11 or 7-10 contiguous basepairs. In specific examples, the bulges are spaced apart by 6, 7, 8, 9, 10, 11, 12, 13, 14, 15, 16, 17, 18, 19, 20 or 21 contiguous basepairs. In a preferred example, all of these basepairs are canonical basepairs. Alternatively, 1, 2, 3, 4, 5 or 6 of the basepairs between the bulges are G:U basepairs. In a preferred example, all of these basepairs are canonical basepairs. Alternatively, 1, 2, 3, 4 or 5 of the basepairs between the bulges are G:U basepairs.

In each of these embodiments of the third aspect, or molecules lacking a loop, the first RNA sequence (E) and the second RNA sequence (G) may both comprise at least 100 contiguous ribonucleotides, or at least 110 or at least 120 contiguous ribonucleotides, or at least 150, or at least 200, or at least 250, or at least 300 contiguous ribonucleotides, preferably to a maximum of 1000 contiguous ribonucleotides, more preferably to a maximum of 800 contiguous ribonucleotides, or even more preferable to a maximum of 600 contiguous ribonucleotides. For example, the first RNA sequence (E) and the second RNA sequence (G) may both comprise contiguous ribonucleotides in the range 100-1000, 100-800, 100-600 contiguous ribonucleotides, or in the range 150-1000, 150-800, 150-600 contiguous ribonucleotides. In preferred embodiments, the length of the sense RNA sequence of the dsRNA region is 87%-97% or 87%-96% the length of the antisense sequence, for example 87%-91% or 87%-90% compared to the antisense sequence. These features are applicable to hairpin RNAs and to dsRNAs formed by annealing of two RNA strands i.e. without a joining loop sequence. Each of these features may also be applied to a second dsRNA region in the precursor RNA molecule, for example in a ledRNA molecule. In these embodiments, the eukaryotic cell in which the precursor RNA molecule (A) is cleaved is preferably a plant cell, a fungal cell, a nematode cell, or an arthropod cell such as an insect, arachnid, or decapod cell, or the target RNA molecule is preferably in a plant cell, a fungal cell, a nematode cell, or an arthropod cell such as an insect, arachnid, or decapod cell.

Each of the embodiments of the third aspect, or the molecules lacking a loop, may have the following feature: the precursor RNA molecule is capable of being cleaved in a eukaryotic cell by one or more ribonucleases (RNases) to produce multiple, different double-stranded product RNA molecules (P) which each consist of a sense RNA sequence (H) of 21 contiguous ribonucleotides from the first RNA sequence (E) and an antisense RNA sequence (J) of 24 contiguous ribonucleotides from the second RNA sequence (G), wherein at least some of the multiple, different double-stranded product RNA molecules (P) have overlapping antisense RNA sequences (J). In a further embodiment, at least some of the multiple, different double-stranded product RNA molecules (P) have non-overlapping antisense RNA sequences (J), where the population of double-stranded product RNA molecules (P) produced from the precursor RNA molecule includes some overlapping and some non-overlapping antisense RNA sequences (J). Preferably, there are more product RNA molecules (P) comprising non-overlapping antisense RNA sequences (J), as readily occurs with longer (>42 basepairs) double-stranded regions (B) where siRNA molecules each consisting of 21 nt sense RNA sequences hybridised to 24 nt antisense RNA sequence are phased along the length of (B).

Each of the embodiments of the third aspect may have the following feature: the precursor RNA molecule is capable of being cleaved in a eukaryotic cell by one or more ribonucleases (RNases) to produce multiple, different double-stranded product RNA molecules (P) which each consist of a sense RNA sequence (H) of 21 contiguous ribonucleotides from a first RNA sequence (E) and an antisense RNA sequence (J) of 24 contiguous ribonucleotides from a second RNA sequence (G), wherein at least some of the multiple, different double-stranded product RNA molecules (P) have non-overlapping antisense RNA sequences (J), preferably adjacent non-overlapping antisense RNA sequences (J).

In an embodiment of the third aspect, the molecule is capable of being cleaved in a eukaryotic cell by one or more ribonucleases (RNases) to produce multiple, different double-stranded product RNA molecules (P) which comprise double-stranded RNA product molecules as defined in two or more embodiments of the third aspect.

The present invention also provides symmetric and asymmetric precursor RNA molecules comprising G:U basepairs in a double-stranded RNA region targeting one or more RNA molecules in an insect cell or fungal cell. Therefore, in a fourth aspect the present invention provides a precursor RNA molecule comprising at least one double-stranded RNA region, wherein:
(i) the double-stranded RNA region comprises:
   (a) a first RNA strand of at least 46 contiguous ribonucleotides which comprises a first RNA sequence of at least 44 contiguous ribonucleotides, and
   (b) a second RNA strand of at least 46 contiguous ribonucleotides which comprises a second RNA sequence of at least 44 contiguous ribonucleotides,
wherein the first RNA sequence hybridises to the second RNA sequence by basepairing between the at least 44 contiguous ribonucleotides of the first RNA sequence and the at least 44 contiguous ribonucleotides of the second RNA sequence, forming at least a part of the double-stranded RNA region,
wherein between 10% and 35% of the ribonucleotides of the first RNA sequence and the second RNA sequence, in total, are basepaired in G:U basepairs,
wherein the second RNA sequence is at least 80% identical to a sequence of at least 44 contiguous ribonucleotides which is fully complementary to a first region of a target RNA molecule in an insect cell; and
(ii) the precursor RNA molecule is capable of being cleaved in an insect cell by one or more Dicers to produce double-stranded product RNA molecules, each independently consisting of a sense RNA sequence of 21 contiguous ribonucleotides from the first RNA sequence and an antisense RNA sequence of 21 contiguous ribonucleotides from the second RNA sequence,
wherein ribonucleotides 1 to 19 of the sense RNA sequence basepair with ribonucleotides 1 to 19 of the antisense RNA sequence in each of the double-stranded product RNA molecules,
wherein the 19 basepairs between the sense RNA sequence and the antisense RNA sequence in each of the double-stranded product RNA molecules comprise, independently, 2, 3, 4, 5, or 6 G:U basepairs,
wherein ribonucleotides 20 and 21 of the sense RNA sequence in each of the double-stranded product RNA molecules form a 2-ribonucleotide unpaired 3′ overhang, and wherein ribonucleotides 20 and 21 of the antisense RNA sequence in each of the double-stranded product RNA molecules form a 2-ribonucleotide unpaired 3′ overhang, and
wherein either (c) at least some of the antisense RNA sequences in the double-stranded product RNA molecules are capable of reducing the expression and/or activity of the target RNA molecule in the insect cell, or
(d) the antisense RNA sequences in the double-stranded product RNA molecules are each, independently, at least 80% identical in sequence to a corresponding region of the complement of the target RNA molecule, or
(e) both (c) and (d).

In a preferred embodiment, the insect cell is a Lepidopteran insect cell, for example of the genus Helicoverpa or Spodoptera, for example, of the species Helicoverpa armigera or Spodoptera frugiperda, and/or the second RNA sequence is at least 90% identical, preferably 100% identical, to the sequence of at least 44 contiguous ribonucleotides which is fully complementary to the first region of the target RNA molecule in an insect cell.

In a fifth aspect the present invention provides a precursor RNA molecule comprising at least one double-stranded RNA region, wherein:
(i) the double-stranded RNA region comprises:
   (a) a first RNA strand of at least 46 contiguous ribonucleotides which comprises a first RNA sequence of at least 44 contiguous ribonucleotides, and
   (b) a second RNA strand of at least 46 contiguous ribonucleotides which comprises a second RNA sequence of at least 44 contiguous ribonucleotides,
wherein the first RNA sequence hybridises to the second RNA sequence by basepairing between the at least 44 contiguous ribonucleotides of the first RNA sequence and the at least 44 contiguous ribonucleotides of the second RNA sequence, forming at least a part of the double-stranded RNA region,
wherein between 10% and 35% of the ribonucleotides of the first RNA sequence and the second RNA sequence, in total, are basepaired in G:U basepairs,
wherein the second RNA sequence is at least 80% identical to a sequence of at least 44 contiguous ribonucleotides which is fully complementary to a first region of a target RNA molecule in a fungal cell; and
(ii) the precursor RNA molecule is capable of being cleaved in the fungal cell by one or more Dicers to produce double-stranded product RNA molecules, each independently consisting of a sense RNA sequence of 21 contiguous ribonucleotides from the first RNA sequence and an antisense RNA sequence of 21 contiguous ribonucleotides from the second RNA sequence,
wherein ribonucleotides 1 to 19 of the sense RNA sequence basepair with ribonucleotides 1 to 19 of the antisense RNA sequence in each of the double-stranded product RNA molecules,
wherein the 19 basepairs between the sense RNA sequence and the antisense RNA sequence in each of the double-stranded product RNA molecules comprise, independently, 2, 3, 4, 5, or 6 G:U basepairs,
wherein ribonucleotides 20 and 21 of the sense RNA sequence in each of the double-stranded product RNA molecules form a 2-ribonucleotide unpaired 3′ overhang, and wherein ribonucleotides 20 and 21 of the antisense RNA sequence in each of the double-stranded product RNA molecules form a 2-ribonucleotide unpaired 3′ overhang, and
wherein either (c) at least some of the antisense RNA sequences in the double-stranded product RNA molecules are capable of reducing the expression and/or activity of the target RNA molecule in the fungal cell, or
(d) the antisense RNA sequences in the double-stranded product RNA molecules are each, independently, at least 80% identical in sequence to a corresponding region of the complement of the target RNA molecule, or
(e) both (c) and (d).

In a preferred embodiment, the fungal cell is a plant pathogenic fungal cell, for example of the genus Fusarium or Verticillium, and/or the second RNA sequence is at least 90% identical, preferably 100% identical, to the sequence of at least 44 contiguous ribonucleotides which is fully complementary to the first region of the target RNA molecule in a fungal cell.

In each embodiment of the fourth and fifth aspects, the features of the first RNA sequence (E) and the second RNA sequence (G) that apply to the precursor RNA molecules of the first, second and third aspects may also apply to the precursor RNA molecules of the fourth and fifth aspects, in particular the length features.

In an embodiment of the asymmetric precursor RNA molecule comprising G:U basepairs, the precursor RNA molecule comprises at least one double-stranded RNA region, wherein:
(i) the double-stranded RNA region comprises:
   (a) a first RNA strand of at least 46 contiguous ribonucleotides which comprises a first RNA sequence of at least 44 contiguous ribonucleotides, and
   (b) a second RNA strand of at least 48 contiguous ribonucleotides which comprises a second RNA sequence of at least 46 contiguous ribonucleotides,
wherein the first RNA sequence hybridises to the second RNA sequence by basepairing between at least 42 ribonucleotides of the at least 44 contiguous ribonucleotides of the first RNA sequence and at least 42 ribonucleotides of the at least 46 contiguous ribonucleotides of the second RNA sequence, forming at least a part of the double-stranded RNA region,
wherein 2, 3, or 4 ribonucleotides of the at least 46 contiguous ribonucleotides of the second RNA sequence are non-basepaired and 0, 1, or 2, respectively, ribonucleotides of the at least 44 contiguous ribonucleotides of the first RNA sequence are non-basepaired in the part of the double-stranded RNA region, forming bulges in the part of the double-stranded RNA region,
wherein each of the bulges is immediately flanked by ribonucleotides which are basepaired in the part of the double-stranded RNA region,
wherein between 10% and 35% of the ribonucleotides of the first RNA sequence and the second RNA sequence, in total, are basepaired in G:U basepairs,
wherein the second RNA sequence is at least 80% identical to a sequence of at least 46 contiguous ribonucleotides which is fully complementary to a first region of a target RNA molecule in an insect cell or a fungal cell; and
(ii) the precursor RNA molecule is capable of being cleaved in the insect cell or fungal cell by one or more Dicers to produce double-stranded product RNA molecules, each independently consisting of a sense RNA sequence of 21 contiguous ribonucleotides from the first RNA sequence and an antisense RNA sequence of 22 contiguous ribonucleotides from the second RNA sequence,
wherein the antisense RNA sequence of the double-stranded product RNA molecules each comprise at least one of the bulges,
wherein the basepairs between the sense RNA sequence and the antisense RNA sequence in each of the double-stranded product RNA molecules comprise, independently, 2, 3, 4, 5, or 6 G:U basepairs,
wherein ribonucleotides 20 and 21 of the sense RNA sequence in each of the double-stranded product RNA molecules form a 2-ribonucleotide unpaired 3′ overhang,
wherein ribonucleotides 21 and 22 of the antisense RNA sequence in each of the double-stranded product RNA molecules form a 2-ribonucleotide unpaired 3′ overhang, and
wherein either (c) at least some of the antisense RNA sequences in the double-stranded product RNA molecules are capable of reducing the expression and/or activity of the target RNA molecule in the insect cell or fungal cell, or
(d) the antisense RNA sequences in the double-stranded product RNA molecules are each, independently, at least 80% identical in sequence to a corresponding region of the complement of the target RNA molecule, or
(e) both (c) and (d).

In another embodiment of the asymmetric precursor RNA molecule comprising G:U basepairs, the precursor RNA molecule comprises at least one double-stranded RNA region, wherein:
(i) the double-stranded RNA region comprises:
   (a) a first RNA strand of at least 46 contiguous ribonucleotides which comprises a first RNA sequence of at least 44 contiguous ribonucleotides, and
   (b) a second RNA strand of at least 50 contiguous ribonucleotides which comprises a second RNA sequence of at least 48 contiguous ribonucleotides,
wherein the first RNA sequence hybridises to the second RNA sequence by basepairing between at least 42 ribonucleotides of the at least 44 contiguous ribonucleotides of the first RNA sequence and at least 42 ribonucleotides of the at least 48 contiguous ribonucleotides of the second RNA sequence, forming at least a part of the double-stranded RNA region,
wherein 4, 5 or 6 ribonucleotides of the at least 48 contiguous ribonucleotides of the second RNA sequence are non-basepaired and 0, 1 or 2, respectively, ribonucleotides of the at least 44 contiguous ribonucleotides of the first RNA sequence are non-basepaired in the part of the double-stranded RNA region, forming bulges in the part of the double-stranded RNA region,
wherein each of the bulges is immediately flanked by ribonucleotides which are basepaired in the part of the double-stranded RNA region,
wherein between 10% and 35% of the ribonucleotides of the first RNA sequence and the second RNA sequence, in total, are basepaired in G:U basepairs,
wherein the second RNA sequence is at least 80% identical to a sequence of at least 48 contiguous ribonucleotides which is fully complementary to a first region of a target RNA molecule in an insect cell or a fungal cell; and
(ii) the precursor RNA molecule is capable of being cleaved in the insect cell or fungal cell by one or more Dicers to produce double-stranded product RNA molecules, each independently consisting of a sense RNA sequence of 21 contiguous ribonucleotides from the first RNA sequence and an antisense RNA sequence of 23 contiguous ribonucleotides from the second RNA sequence,
wherein the antisense RNA sequence of the double-stranded product RNA molecules each comprise at least one of the bulges,
wherein the basepairs between the sense RNA sequence and the antisense RNA sequence in each of the double-stranded product RNA molecules comprise, independently, 2, 3, 4, 5, or 6 G:U basepairs,
wherein ribonucleotides 20 and 21 of the sense RNA sequence in each of the double-stranded product RNA molecules form a 2-ribonucleotide unpaired 3′ overhang,
wherein ribonucleotides 22 and 23 of the antisense RNA sequence in each of the double-stranded product RNA molecules form a 2-ribonucleotide unpaired 3′ overhang, and
wherein either (c) at least some of the antisense RNA sequences in the double-stranded product RNA molecules are capable of reducing the expression and/or activity of the target RNA molecule in the insect cell or fungal cell, or
(d) the antisense RNA sequences in the double-stranded product RNA molecules are each, independently, at least 80% identical in sequence to a corresponding region of the complement of the target RNA molecule, or
(e) both (c) and (d).

In a further embodiment of the asymmetric precursor RNA molecule comprising G:U basepairs, the precursor RNA molecule comprises at least one double-stranded RNA region, wherein:
(i) the double-stranded RNA region comprises:
   (a) a first RNA strand of at least 46 contiguous ribonucleotides which comprises a first RNA sequence of at least 44 contiguous ribonucleotides, and
   (b) a second RNA strand of at least 52 contiguous ribonucleotides which comprises a second RNA sequence of at least 50 contiguous ribonucleotides,
wherein the first RNA sequence hybridises to the second RNA sequence by basepairing between at least 42 ribonucleotides of the at least 44 contiguous ribonucleotides of the first RNA sequence and at least 42 ribonucleotides of the at least 50 contiguous ribonucleotides of the second RNA sequence, forming at least a part of the double-stranded RNA region,
wherein 6, 7, or 8 ribonucleotides of the at least 50 contiguous ribonucleotides of the second RNA sequence are non-basepaired and 0, 1, or 2, respectively, ribonucleotides of the at least 44 contiguous ribonucleotides of the first RNA sequence are non-basepaired in the part of the double-stranded RNA region, forming bulges in the part of the double-stranded RNA region,
wherein each of the bulges is immediately flanked by ribonucleotides which are basepaired in the part of the double-stranded RNA region,
wherein between 10% and 35% of the ribonucleotides of the first RNA sequence and the second RNA sequence, in total, are basepaired in G:U basepairs,
wherein the second RNA sequence is at least 80% identical to a sequence of at least 50 contiguous ribonucleotides which is fully complementary to a first region of a target RNA molecule in an insect cell or a fungal cell; and
(ii) the precursor RNA molecule is capable of being cleaved in the insect cell or fungal cell by one or more Dicers to produce double-stranded product RNA molecules, each independently consisting of a sense RNA sequence of 21 contiguous ribonucleotides from the first RNA sequence and an antisense RNA sequence of 24 contiguous ribonucleotides from the second RNA sequence,
wherein the antisense RNA sequence of the double-stranded product RNA molecules each comprise at least one of the bulges,
wherein the basepairs between the sense RNA sequence and the antisense RNA sequence in each of the double-stranded product RNA molecules comprise, independently, 2, 3, 4, 5, or 6 G:U basepairs,
wherein ribonucleotides 20 and 21 of the sense RNA sequence in each of the double-stranded product RNA molecules form a 2-ribonucleotide unpaired 3′ overhang,
wherein ribonucleotides 23 and 24 of the antisense RNA sequence in each of the double-stranded product RNA molecules form a 2-ribonucleotide unpaired 3′ overhang, and
wherein either (c) at least some of the antisense RNA sequences in the double-stranded product RNA molecules are capable of reducing the expression and/or activity of the target RNA molecule in the insect cell or fungal cell, or
(d) the antisense RNA sequences in the double-stranded product RNA molecules are each, independently, at least 80% identical in sequence to a corresponding region of the complement of the target RNA molecule, or
(e) both (c) and (d).

It is understood in the context of the embodiments of the fourth or fifth aspects or the asymmetric precursor RNA molecules, that at least some, but not necessarily all, of the double-stranded product RNA molecules produced from the precursor RNA molecule have the features recited in part (ii). For example, sense or antisense RNAs of lengths other than 21 ribonucleotides, or 22-, 23- or 24-mers as the case may be, may be produced as well as those of the specified lengths such as ribonucleotides, 21 ribonucleotides.

In an embodiment of the first, second, third, fourth, or fifth aspects, or the embodiments of the asymmetric precursor RNA molecule, the precursor RNA molecule comprises a linking RNA sequence, wherein the linking RNA sequence links either the 3′ end of the first RNA strand to 5′ end of the second RNA strand, or 5′ end of the first RNA strand to the 3′ end of the second RNA strand. In an alternative embodiment, the precursor RNA molecule lacks a linking RNA sequence, i.e. the first RNA strand and the second RNA strands are not covalently linked by a linking RNA sequence. In an embodiment of the first, second, third, fourth, or fifth aspects, or the embodiments of the asymmetric precursor RNA molecule, the precursor RNA molecule comprises a second double-stranded RNA region and a second linking RNA sequence and forms a ledRNA structure.

In an embodiment of the first, second, third, fourth, or fifth aspects, or the embodiments of the asymmetric precursor RNA molecule, one or more or all of the following may apply:
(i) the linking RNA sequence comprises a sequence of at least 50 contiguous ribonucleotides which is at least 90% identical to a second region of the target RNA molecule in the eukaryotic cell such as a plant cell, nematode cell, insect cell or fungal cell, and/or the linking RNA sequence comprises a sequence of at least 50 contiguous ribonucleotides which is at least 90% identical to the complement of a second region of the target RNA molecule in the eukaryotic cell such as a plant cell, nematode cell, insect cell or fungal cell,
(ii) the precursor RNA molecule further comprises a sequence of at least 50 contiguous ribonucleotides which is at least 90% identical to a second region of the target RNA molecule in the eukaryotic cell such as a plant cell, nematode cell, insect cell or fungal cell, covalently linked to the 5′ or 3′ end of the first RNA strand or to 5′ or 3′ end of the second RNA strand, and/or a sequence of at least 50 contiguous ribonucleotides which is at least 90% identical to the complement of a second region of the target RNA molecule in the eukaryotic cell such as a plant cell, nematode cell, insect cell or fungal cell, covalently linked to the 5′ or 3′ end of the first RNA strand or to 5′ or 3′ end of the second RNA strand,
(iii) the linking RNA sequence comprises a single-stranded RNA sequence of at least 44 contiguous ribonucleotides which is identical to a region of either the first RNA sequence or the second RNA sequence,
(iv) the precursor RNA molecule comprises the linking RNA sequence and is encoded by a polynucleotide which lacks an intron in its region encoding the linking RNA sequence,
(v) the first RNA sequence and second RNA sequence are identical in length across the full length of the double-stranded RNA region,
(vi) in the embodiments of the asymmetric precursor RNA molecule, the first RNA sequence and second RNA sequence are different in length across the full length of the double-stranded RNA region, either the first RNA sequence is longer than the second RNA sequence or, preferably, the first RNA sequence is shorter than the second RNA sequence,
(vii) the first RNA sequence varies from the first region of the target RNA molecule only by the substitution of at least some A ribonucleotides with G ribonucleotides, or at least some C ribonucleotides with U ribonucleotides, or a combination of at least some A ribonucleotides with G ribonucleotides and at least some C ribonucleotides with U ribonucleotides, or wherein some but not all of the A ribonucleotides are substituted with G ribonucleotides, or some but not all C ribonucleotides are substituted with U ribonucleotides, or a combination of some but not all A ribonucleotides with G ribonucleotides and some but not all C ribonucleotides with U ribonucleotides, and optionally wherein the second RNA sequence is identical in sequence to the complement of the first region of the target RNA molecule,
(viii) the second RNA sequence varies from the complement of the first region of the target RNA molecule only by the substitution of at least some A ribonucleotides with G ribonucleotides, or at least some C ribonucleotides with U ribonucleotides, or a combination of at least some A ribonucleotides with G ribonucleotides and at least some C ribonucleotides with U ribonucleotides, or wherein some but not all of the A ribonucleotides are substituted with G ribonucleotides, or some but not all C ribonucleotides are substituted with U ribonucleotides, or a combination of some but not all A ribonucleotides with G ribonucleotides and some but not all C ribonucleotides with U ribonucleotides, and optionally wherein the first RNA sequence is identical in sequence to the first region of the target RNA molecule,
(ix) at least some of the double-stranded product RNA molecules have non-overlapping antisense RNA sequences, preferably adjacent non-overlapping antisense RNA sequences, relative to the target RNA molecule,
(x) the sense RNA sequence of each of the double-stranded product RNA molecules varies from their corresponding sequence in the first region of the target RNA molecule only by the substitution of at least some A ribonucleotides with G ribonucleotides, or at least some C ribonucleotides with U ribonucleotides, or a combination of at least some A ribonucleotides with G ribonucleotides and at least some C ribonucleotides with U ribonucleotides, or wherein some but not all of the A ribonucleotides are substituted with G ribonucleotides, or some but not all C ribonucleotides are substituted with U ribonucleotides, or a combination of some but not all A ribonucleotides with G ribonucleotides and some but not all C ribonucleotides with U ribonucleotides,
(xi) the antisense RNA sequence of each of the double-stranded product RNA molecules varies from a corresponding sequence in the complement of the first region of the target RNA molecule only by the substitution of at least some A ribonucleotides with G ribonucleotides, or at least some C ribonucleotides with U ribonucleotides, or a combination of at least some A ribonucleotides with G ribonucleotides and at least some C ribonucleotides with U ribonucleotides, or wherein some but not all of the A ribonucleotides are substituted with G ribonucleotides, or some but not all C ribonucleotides are substituted with U ribonucleotides, or a combination of some but not all A ribonucleotides with G ribonucleotides and some but not all C ribonucleotides with U ribonucleotides,
(xii) the percentage of G ribonucleotides in the first RNA sequence is between 26-40% of the total number of ribonucleotides in the first RNA sequence, and/or the percentage of G ribonucleotides in the second RNA sequence is between 26-40% of the total number of ribonucleotides in the second RNA sequence,
(xiii) the percentage of U ribonucleotides in the first RNA sequence is between 26-40% of the total number of ribonucleotides in the first RNA sequence, and/or the percentage of U ribonucleotides in the second RNA sequence is between 26-40% of the total number of ribonucleotides in the second RNA sequence,
(xiv) the double-stranded RNA region has at most 18 contiguous canonical basepairs,
(xv) the precursor RNA molecule is capable of being made enzymatically by transcription in vitro or in a cell, or both.

In embodiments, combinations of the above features (i) to (xv) apply. Preferred combinations include (i) and (ii), (i) and (iv), (i) and any one of (v) to (xv), (ii) and (iii), (ii) and (iv), (ii) and any one of (v) to (xv), (iii) and (iv), (iii) and any one of (v) to (xv), (iv) and any one of (v) to (xv), (v) and (vii), (v) and (viii), (v) and (x), (v) and (xi), (v) and (xii), (v) and (xiii), (v) and (xiv), (vi) and (vii), (vi) and (viii), (vi) and (x), (vi) and (xi), (vi) and (xii), (vi) and (xiii), (vi) and (xiv), (vii) and (viii), (vii) and (x), (vii) and (xi), (vii) and (xii), (vii) and (xiii), (vii) and (xiv), (viii) and (x), (viii) and (xi), (viii) and (xii), (viii) and (xiii), (viii) and (xiv), (x) and (xi), (x) and (xii), (x) and (xiii), (x) and (xiv), (xi) and (xii), (xi) and (xiii), (xi) and (xiv), (xii) and (xiii), (xii) and (xiv).

In an embodiment of the first, second, third, fourth, or fifth aspects, or the embodiments of the asymmetric precursor RNA molecule, the precursor RNA molecule comprises a linking RNA sequence which comprises a sequence of at least 50 contiguous ribonucleotides which is at least 90% identical, preferably at least 95% or 100% identical, to a second region of the target RNA molecule. The second region of the target RNA molecule is preferably 3′ of the first region of the target RNA molecule. In analogous fashion, the linking RNA sequence comprises a sequence of at least 50 contiguous ribonucleotides which is at least 90% identical, preferably at least 95% or 100% identical, to the complement of the second region of the target RNA molecule.

In an embodiment of the first, second, third, fourth, or fifth aspects, or the embodiments of the asymmetric precursor RNA molecule, the precursor RNA molecule further comprises a sequence of at least 50 contiguous ribonucleotides which is at least 90% identical, preferably at least 95% or 100% identical, to a second region of the target RNA molecule in the eukaryotic cell such as a plant cell, nematode cell, insect cell or fungal cell, covalently linked to the 5′ or 3′ end of the first RNA strand or to 5′ or 3′ end of the second RNA strand, and/or a sequence of at least 50 contiguous ribonucleotides which is at least 90% identical, preferably at least 95% or 100% identical, to the complement of a second region of the target RNA molecule, covalently linked to the 5′ or 3′ end of the first RNA strand or to 5′ or 3′ end of the second RNA strand.

In an embodiment of the first, second, third, fourth, or fifth aspects, or the embodiments of the asymmetric precursor RNA molecule, the linking RNA sequence comprises a single-stranded RNA sequence of at least 44, or at least 50, or at least 100, contiguous ribonucleotides which is identical to a region of either the first RNA sequence or the second RNA sequence of the double-stranded RNA region. Such a second copy of the RNA sequence in the precursor RNA molecule, in a single-stranded form, is considered to increase the inhibitory activity of the precursor RNA molecule, for example by increasing production of secondary siRNAs.

In an embodiment of the first, second, third, fourth, or fifth aspects, or the embodiments of the asymmetric precursor RNA molecule, the precursor RNA molecule comprises a linking RNA sequence and is encoded by a polynucleotide which lacks an intron in its region encoding the linking RNA sequence. That is, the initial transcript from the polynucleotide lacks an intron sequence, instead has a linking RNA sequence without an intron.

In an embodiment of the fourth or fifth aspects as applied to a symmetric precursor RNA molecule, also to the embodiments of the asymmetric precursor RNA molecule of the first, second, third, fourth, or fifth aspects, the first RNA sequence and second RNA sequence are identical in length across the full length of the double-stranded RNA region. That is, if there are non-basepaired ribonucleotides in the first and second RNA sequences, the number of non-basepaired ribonucleotides in the first RNA sequence is equal to the number of non-basepaired ribonucleotides in the second RNA sequence. This can be achieved through insertions and/or deletions in one or both sequences.

In embodiments of the asymmetric precursor RNA molecules of the first, second, third, fourth, or fifth aspects, or the embodiments of the asymmetric precursor RNA molecule, the first RNA sequence and second RNA sequence are different in length across the full length of the double-stranded RNA region, either the first RNA sequence is longer than the second RNA sequence or, preferably, the first RNA sequence is shorter than the second RNA sequence. In an embodiment, the different lengths are due at least in part, preferably entirely, to non-basepaired ribonucleotides in the second RNA sequence that bulge out from the double-stranded RNA region. In an embodiment, the sense sequence has a length which is between 87% and 97%, or 87% and 96%, or 91% and 97%, or 91% and 96%, or more preferably between 94% and 97% or 94% and 96% of the length of the antisense sequence, or the length of the sense sequence is about 21/22, 21/23, or 21/24 of the length of the antisense sequence, calculated as a fraction.

In an embodiment of the first, second, third, fourth, or fifth aspects, or the embodiments of the asymmetric precursor RNA molecule, the first RNA sequence and/or the second RNA sequence varies from the first region of the target RNA molecule or its complement, respectively, only by the substitution of at least some A ribonucleotides with G ribonucleotides, preferably 40-60% of the A ribonucleotides, or at least some C ribonucleotides with U ribonucleotides, preferably 40-60% of the C ribonucleotides, or a combination of at least some A ribonucleotides, preferably 40-60% of the A ribonucleotides, with G ribonucleotides and at least some C ribonucleotides, preferably 40-60% of the C ribonucleotides, with U ribonucleotides. In an embodiment, some but not all of the A ribonucleotides are substituted with G ribonucleotides, or some but not all C ribonucleotides are substituted with U ribonucleotides, or a combination of some but not all A ribonucleotides with G ribonucleotides and some but not all C ribonucleotides with U ribonucleotides. In a preferred embodiment, the second RNA sequence is identical in sequence to the complement of the first region of the target RNA molecule, i.e. has no substitutions. These features are also applicable to double-stranded product RNA molecules produced from the precursor RNA molecule.

In an embodiment of the first, second, third, fourth, or fifth aspects, or the embodiments of the asymmetric precursor RNA molecule, the ribonucleotide substitutions result in G:U basepairs in the double-stranded RNA region, for example between 10% and 35%, between 10% and 30%, or between 10% and 25% of the ribonucleotides in the double-stranded RNA region, in total, and/or the part of the double-stranded RNA region, in total, form a G:U basepair. In an embodiment, between 15% and 35%, between 15% and 30%, or between 15% and 25% of the ribonucleotides in the double-stranded RNA region, in total, and/or the part of the double-stranded RNA region, in total, form a G:U basepair. In an embodiment, between 17% and 35%, between 17% and 30%, or between 17% and 25% of the ribonucleotides in the double-stranded RNA region, in total, and/or the part of the double-stranded RNA region, in total, form a G:U basepair. In an embodiment, about 12%, about 15%, about 20%, about 25%, or about 30% of the ribonucleotides in the double-stranded RNA region, in total, and/or the part of the double-stranded RNA region, in total, form a G:U basepair.

In an embodiment of the first, second, third, fourth, or fifth aspects, or the embodiments of the asymmetric precursor RNA molecule, the percentage of G ribonucleotides in the first RNA sequence is increased by A to G substitutions. For example, between 26-40% of the total number of ribonucleotides in the first RNA sequence are G ribonucleotides, preferably 28-40%, 30-40%, 32-40%, 34-40%, or 26-38%, 26-36%, or 26-34% of the total number of ribonucleotides in the first RNA sequence are G ribonucleotides. In an embodiment, the percentage of G ribonucleotides in the second RNA sequence is increased by A to G substitutions, for example, between 26-40% of the total number of ribonucleotides in the second RNA sequence are G ribonucleotides, preferably 28-40%, 30-40%, 32-40%, 34-40%, or 26-38%, 26-36%, or 26-34% of the total number of ribonucleotides in the first RNA sequence are G ribonucleotides. In this context, substitutions are relative to the sequence of the region of the target RNA molecule or its complement. In an embodiment, either the first or second RNA sequence, or both, lack A to G substitutions relative to the region of the target RNA molecule or its complement, respectively.

In an embodiment of the first, second, third, fourth, or fifth aspects, or the embodiments of the asymmetric precursor RNA molecule, the percentage of U ribonucleotides in the first RNA sequence is increased by C to U substitutions. For example, between 26-40% of the total number of ribonucleotides in the first RNA sequence are U ribonucleotides, preferably 28-40%, 30-40%, 32-40%, 34-40%, or 26-38%, 26-36% or 26-34% of the total number of ribonucleotides in the first RNA sequence are U ribonucleotides. In an embodiment, the percentage of U ribonucleotides in the second RNA sequence is increased by C to U substitutions, for example, between 26-40% of the total number of ribonucleotides in the second RNA sequence are G ribonucleotides, preferably 28-40%, 30-40%, 32-40%, 34-40%, or 26-38%, 26-36% or 26-34% of the total number of ribonucleotides in the first RNA sequence are G ribonucleotides. In this context, substitutions are relative to the sequence of the region of the target RNA molecule or its complement. In an embodiment, either the first or second RNA sequence, or both, lack C to U substitutions relative to the region of the target RNA molecule or its complement, respectively.

In an embodiment of the first, second, third, fourth, or fifth aspects, or the embodiments of the asymmetric precursor RNA molecule, the double-stranded RNA region has at most 18 contiguous canonical basepairs, preferably at most 17, at most 16, at most 15, at most 14, at most 13, at most 12, at most 11, at most 10, at most 9, at most 8, or at most 7 contiguous canonical basepairs. That is, these numbers represent the maximum number of contiguous canonical basepairs for the longest subregion of contiguous canonical basepairing in the dsRNA region. The calculation of the number of contiguous canonical basepairs ignores the presence of any non-basepaired ribonucleotides in the double-stranded region. Reducing the number of contiguous canonical basepairs in any subregion can be achieved by regular spacing of A to G and C to U substitutions in either or both sequences.

In an embodiment of the fourth or fifth aspects, or the embodiments of the asymmetric precursor RNA molecule of the first, second, third, fourth, or fifth aspects, or the embodiments of the asymmetric precursor RNA molecule, at least some of the antisense RNA sequences in the double-stranded product RNA molecules produced from the precursor RNA molecule basepair along the full length of the antisense RNA sequences to the region of the target RNA molecule, preferably basepair along the full length by canonical basepairs.

In an embodiment of the fourth or fifth aspects, or the embodiments of the asymmetric precursor RNA molecule of the first, second, third, fourth, or fifth aspects, at least some of the antisense RNA sequences in the double-stranded product RNA molecules produced from the precursor RNA molecule are capable of reducing the expression and/or activity of the target RNA molecule in the eukaryotic cell such as a plant cell, nematode cell, insect cell or fungal cell. In an embodiment, the antisense RNA sequences in the double-stranded product RNA molecules produced from the precursor RNA molecule are capable of reducing the expression and/or activity of multiple, different target RNA molecules in the eukaryotic cell such as a plant cell, nematode cell, insect cell or fungal cell, wherein the different target RNA molecules are unrelated in sequence.

In an embodiment, the reduction in expression and/or activity of the target RNA molecule(s) in the insect cell results in death of larvae of the insect, or the mortality rate is increased relative to the use of antisense RNA sequences produced from a corresponding precursor RNA molecule with only canonical basepairing. For example, 87.5-100% of the insect larvae that ingest the precursor RNA molecule and/or the antisense RNA sequences are killed. In an embodiment, the reduction in expression and/or activity of the target RNA molecule(s) in the fungal cell results in decreased symptoms and/or increased resistance to fungal infection relative to the use of antisense RNA sequences produced from a corresponding precursor RNA molecule with only canonical basepairing.

In an embodiment of the ledRNA molecules of the first, second, third, fourth, or fifth aspects, or the embodiments of the asymmetric precursor RNA molecule, either the sense RNA sequences of the two double-stranded RNA regions are contiguous relative to the target RNA molecule, or the antisense RNA sequences of the two double-stranded RNA regions are contiguous relative to the complement of the target RNA molecule. In an embodiment, the two double-stranded RNA regions are capable of being cleaved by a Dicer to produce double-stranded product RNA molecules which comprise antisense RNA sequences which hybridise to one region of a target RNA molecule or to different, non-contiguous regions of the target RNA molecule. In an embodiment, wherein the two double-stranded RNA regions are capable of being cleaved by a Dicer to produce double-stranded product RNA molecules which comprise antisense RNA sequences which hybridise to regions of different target RNA molecules, or to corresponding regions in a family of target RNA molecules. In an embodiment, the second double-stranded region lacks non-basepaired ribonucleotide bulges.

In an embodiment of the ledRNA molecules of the first, second, third, fourth, or fifth aspects, or the embodiments of the asymmetric precursor RNA molecule, the second double-stranded region comprises a third RNA sequence of at least 23 contiguous ribonucleotides and a fourth RNA sequence of at least 26 contiguous ribonucleotides, wherein the third RNA sequence hybridises to the fourth RNA sequence by basepairing between at least 20 ribonucleotides of the at least 23 contiguous ribonucleotides of the third RNA sequence and at least 20 ribonucleotides of the at least 26 contiguous ribonucleotides of the fourth RNA sequence, forming at least a part of the second double-stranded RNA region,
wherein 1, 2, 3, 4, 5, or 6 ribonucleotides of the at least 26 contiguous ribonucleotides of the fourth RNA sequence are non-basepaired and 0, 1, 2, or 3 ribonucleotides of the at least 23 contiguous ribonucleotides of the first RNA sequence are non-basepaired in the part of the double-stranded RNA region, forming bulges in part of the second double-stranded RNA region,
wherein each of the bulges is immediately flanked by ribonucleotides which are basepaired in the part of the second double-stranded RNA region,
wherein the fourth RNA sequence is at least 80% identical to a sequence of at least 26 contiguous ribonucleotides which is fully complementary to a region of a target RNA molecule in eukaryotic cell such as a plant cell, nematode cell, insect cell or fungal cell.

In an embodiment, (i) the first RNA sequence and/or the third RNA sequence differs from a corresponding wild-type RNA sequence in the target RNA molecule by deletion of one or more ribonucleotides from the corresponding wild-type RNA sequence to make the first or third RNA sequence, and/or (ii) the second RNA sequence and/or the fourth RNA sequence differs from a fully complementary sequence to the corresponding wild-type RNA sequence in the target RNA molecule by insertion of one or more ribonucleotides into the fully complementary sequence to make the second or fourth RNA sequence, preferably (i). In an embodiment, the deletion of ribonucleotides from the corresponding wild-type RNA sequence occurs at one or more or all of the ribonucleotide positions corresponding to the non-basepaired ribonucleotides in the second or fourth RNA sequence. In an embodiment, the precursor RNA molecule is capable of being cleaved in eukaryotic cell such as a plant cell, nematode cell, insect cell or fungal cell by one or more Dicers to produce any of the double-stranded product RNA molecules defined in any of the first, second, third, fourth, or fifth aspects.

In an embodiment of the first, second, third, fourth, or fifth aspects, or the embodiments of the asymmetric precursor RNA molecule, the precursor RNA molecule has a single double-stranded RNA region and, optionally, the part of the double-stranded RNA region extends to encompass the whole double-stranded RNA region.

In an embodiment of the first, second, third, fourth, or fifth aspects, or the embodiments of the asymmetric precursor RNA molecule, all of the ribonucleotides of the antisense RNA sequences are capable of basepairing to ribonucleotides in the region of the target RNA molecule.

In an embodiment of the first, second, third, fourth, or fifth aspects, or the embodiments of the asymmetric precursor RNA molecule, the precursor RNA molecule is produced in a plant cell or a microbial cell such as a yeast cell by transcription of an exogenous polynucleotide.

Each of the embodiments of the first, second and third aspects may have the following feature: the antisense RNA sequence (J) from at least one of the product RNA molecules (P) produced from the precursor RNA molecule is capable of hybridising to a region (R) of a target RNA molecule in a eukaryotic cell through at least all of ribonucleotides 2 to 8 of the antisense RNA sequence (J) basepairing with ribonucleotides within the region (R) of the target RNA molecule. The hybridisation may be through at least all of ribonucleotides 2 to 10 or 2 to 11 of the antisense RNA sequence (J).

In an embodiment, including for the immediately preceding paragraph, the eukaryotic cell is a vertebrate animal cell such as a mammalian animal cell, or a non-mammalian vertebrate animal cell, where reduction of activity of the target RNA molecule may be primarily through an inhibition of translation of the target RNA molecule. Where basepairing to the target RNA molecule occurs through a longer area than ribonucleotides 2 to 11, the reduction of activity may be through inhibition of translation and/or cleavage of the target RNA molecule.

Preferably, most of the antisense RNA sequences (J) from the product RNA molecules (P) produced from the precursor RNA molecule are capable of hybridising to a region (R), or more than one region (R), of the target RNA molecule. More preferably, all of the antisense RNA sequences (J) from the product RNA molecules (P) produced from the precursor RNA molecule are capable of hybridising to a region (R), or more than one region, of the target RNA molecule.

In each of the embodiments of the first, second, third, fourth, or fifth aspects, or the embodiments of the asymmetric precursor RNA molecule, at least 50%, at least 60%, at least 70%, at least 75%, at least 80%, at least 85%, at least 90%, or at least 95%, of the ribonucleotides of one or more or all of the antisense RNA sequences (J) are capable of basepairing to ribonucleotides of a region (R) of a target RNA molecule in a eukaryotic cell. Preferably, multiple, non-overlapping antisense RNA sequences (J) are capable of basepairing to ribonucleotides of the region (R) of the target RNA molecule or to multiple regions (R) of the target molecule, or to each of multiple target RNA molecules with any of these minimum percentages.

In an embodiment, all of the ribonucleotides of the antisense RNA sequence (J) are capable of basepairing to ribonucleotides of a region (R) of a target RNA molecule in a eukaryotic cell. Preferably, this feature occurs for each of multiple, non-overlapping antisense RNA sequences (J) that basepair to ribonucleotides of the region (R) of the target RNA molecule or to multiple regions (R) of the target molecule, or to multiple target RNA molecules. This can readily be achieved through the use of longer antisense sequences, at least 50 nt or at least 100 nt in length, in the precursor RNA molecule that are fully complementary to the target RNA molecule. Preferably, all of the ribonucleotides of multiple, different antisense RNA sequences (J) are capable of basepairing to ribonucleotides across a length of a region (R) of a target RNA molecule of at least 150, or at least 200, or at least 250, or at least 300 ribonucleotides, preferably to a maximum of 1000 ribonucleotides, more preferably to a maximum of 800 ribonucleotides, or even more preferably to a maximum of 600 ribonucleotides. For example, preferably all of the ribonucleotides of multiple, different antisense RNA sequences (J) are capable of basepairing to ribonucleotides across a length of a region (R) of a target RNA molecule in the range 100-1000, 100-800, or 100-600 ribonucleotides, or in the range 150-1000, 150-800, or 150-600 ribonucleotides of the target RNA molecule.

Each of the embodiments of the first, second, third, fourth, or fifth aspects, or the embodiments of the asymmetric precursor RNA molecule may have the following feature: the basepairing to ribonucleotides of the region (R) of the target RNA molecule comprises one or more G:U basepairs, preferably 2, 3, 4 or 5 G:U basepairs.

Each of the embodiments of the first, second, third, fourth, or fifth aspects, or the embodiments of the asymmetric precursor RNA molecule may have the following feature: the basepairing to ribonucleotides of the region (R) of the target RNA molecule comprises only canonical basepairs.

Each of the embodiments of the first, second, third, fourth, or fifth aspects, or the embodiments of the asymmetric precursor RNA molecule may have one or more of the following features, where applicable: the region (R) of the target RNA molecule has a length of 22-30, 23-30, 23-33, 24-30, 30-50, 34-200, 50-100, 100-600, or 100-1000 ribonucleotides, and/or the length of the antisense sequence of the dsRNA region is 22-30, 23-30, 23-33, 24-30, 30-50, 34-200, 50-100, 100-600, or 100-1000 ribonucleotides, and/or the sense sequence of the dsRNA region of the precursor RNA molecule within those ranges is shorter than the corresponding antisense sequence, preferably the sense sequence is shorter than the corresponding antisense sequence entirely because of the presence of non-basepaired ribonucleotides in the antisense sequence that bulge from the dsRNA region or the product RNA molecule(s) (P), more preferably the sense sequence has a length which is between 87%-97%, or 87%-96%, or 91%-97%, or 91%-96%, or more preferably 94%-97% or 94%-96% of the length of the antisense sequence, or the length of the sense sequence is about 21/22, 21/23 or 21/24 of the length of the antisense sequence, calculated as a fraction. Preferably, the antisense sequence in the precursor RNA molecule is fully complementary to the target RNA molecule along at least that length.

Each of the embodiments of the first, second, third, fourth, or fifth aspects, or the embodiments of the asymmetric precursor RNA molecule may have the following feature: the precursor RNA molecule comprises two or more different, double-stranded RNA regions (B), wherein each double-stranded RNA region (B) is independently defined herein. Examples of this embodiment are the ledRNA structures shown schematically in FIG. 1.

In an embodiment, the two or more different, double-stranded RNA regions (B) are contiguous with regard to the sequence of the target RNA molecule.

In an alternative embodiment, the two different, double-stranded RNA regions (B) are linked covalently through one or two linking RNA sequence(s). In this context, when two linking RNA sequences are present, one links 3′ end of one strand of the first of the double-stranded regions to 5′ end of one strand of the second double-stranded region, and the other linking RNA sequence links 3′ end of the other strand of the second double-stranded region to 5′ end of the other strand of the first double-stranded region, effectively forming a longer double-stranded region in the precursor RNA molecule.

In an embodiment, one or both of the linkers comprise or consist of ribonucleotides that are non-basepaired, preferably that form one or more bulges or loops in the precursor RNA molecule.

Each of the embodiments of the first, second, third, fourth, or fifth aspects, or the embodiments of the asymmetric precursor RNA molecule may have the following feature, where applicable: the two or more different, double-stranded RNA regions (B) are capable of being cleaved by one or more RNases to produce product RNA molecules (P) which comprise antisense RNA sequences (J) which hybridise to one region (R) of a target RNA molecule or to different, non-contiguous regions (R) of the target RNA molecule.

Each of the embodiments of the first, second, third, fourth, or fifth aspects, or the embodiments of the asymmetric precursor RNA molecule may have the following feature, where applicable: the two or more different, double-stranded RNA regions (B) are capable of being cleaved by one or more RNases to produce product RNA molecules (P) which comprise antisense RNA sequences (J) which hybridise to regions (R) of different target RNA molecules, or to corresponding regions (R) in a family of target RNA molecules.

Each of the embodiments of the first, second, third, fourth, or fifth aspects, or the embodiments of the asymmetric precursor RNA molecule, may have the following feature: the target RNA molecule encodes one or more protein(s). Alternatively, the target RNA molecule does not encode a protein, for example the target RNA is a miRNA.

Each of the embodiments of the first, second and third aspects, or the embodiments of the asymmetric precursor RNA molecule, may have the following feature: the eukaryotic cell is a plant cell, an animal cell, or a fungal cell, preferably a plant cell, an arthropod cell such as an insect, arachnid, or decapod cell, a nematode cell, or a fungal cell. Preferred insect cells are from the orders Lepidoptera, Coleoptera, Diptera and Hemiptera. Other preferred arthropods include those in the Order Arachnida such as spiders and ticks, or Decapoda such as prawns. In an embodiment, the target RNA molecule is an RNA molecule of a viral pathogen of the eukaryotic cell or organism such as the plant, insect, or the decapod. In embodiments of the first, second, and third aspects where the double-stranded region (B) comprises between 20 and 30 basepairs, where the basepairs are all canonical basepairs, or where the basepairs include one or more G:U basepairs, the eukaryotic cell may be a vertebrate animal cell such as a mammalian cell, a human cell, or a non-human mammalian cell, or a non-mammalian vertebrate animal cell. In embodiments of the first, second and third aspects where the double-stranded region (B) comprises between 31 and 50 basepairs and where the eukaryotic cell is a vertebrate animal cell, it is preferred that between 10% and 40%, preferably between 16% and 30% or between 16% and 25%, of the basepairs are G:U basepairs. More preferred, in the context of these cells, is that the precursor RNA molecule comprises a ledRNA structure in addition to having the G:U basepairs. In an embodiment, the vertebrate animal cell is a mammalian cell, a human cell, a non-human mammalian cell or a non-mammalian vertebrate animal cell. In an embodiment, the non-mammalian vertebrate animal cell is a bird cell or a fish cell. The vertebrate animal cell, mammalian cell, human cell or non-human cell may be a cell in culture or in vitro. The cell may be used in a screening assay to identify suitable target RNA molecules.

In an embodiment, applicable to each of the embodiments of the first, second, third, fourth, or fifth aspects, or the embodiments of the asymmetric precursor RNA molecule, the target RNA molecule, or multiple different target RNA molecules, is in a eukaryotic cell which is a plant cell, an animal cell or a fungal cell, preferably a plant cell, an arthropod cell, a nematode cell or a fungal cell. The animal cell may be an arthropod cell such as an insect, arachnid, or decapod cell. Any of these cells may be cells in cell culture.

In an embodiment, applicable to each of the embodiments of the first, second, third, fourth, or fifth aspects, or the embodiments of the asymmetric precursor RNA molecule, the precursor RNA molecule is present in the eukaryotic cell and/or which is produced in the eukaryotic cell, optionally is cleaved by one or more RNases in the eukaryotic cell to produce the one or more double-stranded product RNA molecule(s) (P). Furthermore, the target RNA molecule may be in the same cell. Alternatively, the target RNA molecule is not in the same cell, or has not yet entered the cell e.g. the target RNA molecule is from a viral pathogen which may or may not enter the cell. For example, the precursor RNA molecule may be produced in a microbial cell such as a bacterial cell or a yeast cell, for example Saccharomyces cerevisiae, and applied, with or without extraction of the RNA from the microbial cell, to the cells or organism comprising the target RNA molecule. In an embodiment, the microbial cell is ingested by the target organism and the precursor RNA molecule and/or the siRNA products from the precursor RNA molecule are released from the microbial cell.

In an embodiment, applicable to each of the embodiments of the first, second, third, fourth, or fifth aspects, or the embodiments of the asymmetric precursor RNA molecule, the one or more double-stranded region(s) (B) comprise bulges which are evenly spaced apart along most or all of each double-stranded region (B). For example, the bulges for the Δ22 modification are spaced apart on average about one ribonucleotide bulge in the second RNA sequence (G) about every 22nd ribonucleotide, applicable to longer sequences (G). Analogously, the bulges for the Δ23 or Δ24 modifications are spaced apart on average about two or three bulging antisense ribonucleotides, respectively, in the second RNA sequence (G) about every 23 or 24 ribonucleotides. In this context, “about” means+/−10%, preferably +/−5%.

In an embodiment, applicable to each of the embodiments of the first, second, third, fourth, or fifth aspects, or the embodiments of the asymmetric precursor RNA molecule, each double-stranded region (B) comprises a bulge which is closer to the linking RNA sequence (L) than any other bulge in the double-stranded region (B), wherein said closer bulge and the linking RNA sequence (L) are separated by at least four or more contiguous intervening basepairs, preferably at least 5, at least 6, at least 7, at least 8, at least 9 or at least 10 contiguous intervening basepairs, more preferably by 5-15, 5-14, 5-13, 5-12, or 5-11 contiguous intervening basepairs.

In an embodiment, applicable to each of the embodiments of the first, second, third, fourth, or fifth aspects, or the embodiments of the asymmetric precursor RNA molecule, the precursor RNA molecule comprises a terminal basepair at a junction of the double-stranded region (B) and the linking RNA sequence (L), wherein the terminal basepair comprises a U ribonucleotide as the last 3′ ribonucleotide in the first RNA sequence (E) or the first 5′ ribonucleotide in the second RNA sequence (G), preferably the first 5′ ribonucleotide in the second RNA sequence (G) is a U ribonucleotide.

In an embodiment, the terminal basepair at the junction of the double-stranded region (B) and the linking RNA sequence (L) is an A:U basepair or a G:U basepair, preferably a G:U basepair, more preferably the U is in the second RNA sequence (G).

In an embodiment, applicable to each of the embodiments of the first, second, third, fourth, or fifth aspects, or the embodiments of the asymmetric precursor RNA molecule, the precursor RNA molecule has a single linking RNA sequence (L), thereby forming a hairpin RNA (hpRNA) structure. In an embodiment, the linking RNA sequence joins 3′ end of the first RNA strand (D) and 5′ end of the second RNA strand (F). Alternately, the linking RNA sequence joins 3′ end of the second RNA strand (F) and 5′ end of the first RNA strand (D). Presenting this alternate structure visually, the loop (L) would appear at the left-hand side of the schematic diagrams in FIG. 53, and the order of transcription is second RNA strand (F)-loop (L)-first RNA strand (D).

Alternatively, the invention provides precursor RNA molecules (A) which are the same as the precursor RNA molecules (A) of the first, second, third, fourth, or fifth aspects, or the embodiments of the asymmetric precursor RNA molecule, except that they lack a linking RNA sequence (L). That is, the precursor RNA molecule is comprised of two RNA strands that anneal together. In an embodiment, the precursor RNA molecules (A) lacking the (L) comprise at least 44 basepairs, preferably at least 50 basepairs, more preferably at least 100 basepairs of the second RNA strand (F) is at least 100 ribonucleotides long. All of the features of the embodiments of the first, second and third aspects are applicable to these precursor RNA molecules (A) lacking the (L), singly or in combinations.

In an embodiment, applicable to each of the embodiments of the first, second, third, fourth, or fifth aspects, or the embodiments of the asymmetric precursor RNA molecule, the precursor RNA molecule has two double-stranded regions (B) and two linking RNA sequences (L) forming a ledRNA structure (also known as a dumbbell structure). All of the features of the embodiments of the ledRNA structures as described herein are applicable here, either singly or in combination.

In an embodiment, applicable to each of the embodiments of the hairpin RNAs or ledRNAs of the first, second, third, fourth, or fifth aspects, or the embodiments of the asymmetric precursor RNA molecule, the precursor RNA molecule comprises a terminal basepair at a junction of the double-stranded region (B) and the linking RNA sequence (L), wherein the terminal basepair is the first basepair of part (C) of the double-stranded RNA region.

In an embodiment, applicable to each of the embodiments of the hairpin RNAs or ledRNAs of the first, second, third, fourth, or fifth aspects, or the embodiments of the asymmetric precursor RNA molecule, the precursor RNA molecule comprises at least one linking RNA sequence (L) which is between 4 and 2000 ribonucleotides in length, preferably between 4 and 1000 ribonucleotides in length, more preferably between 4 and 200 ribonucleotides or 4 and 50 ribonucleotides in length and most preferably between 4 and 20 nucleotides in length, preferably wherein all of the linking RNA sequences in the precursor RNA molecule have the aforesaid length.

In an embodiment, applicable to each of the hairpin RNAs or ledRNAs of the embodiments of the first, second, third, fourth, or fifth aspects, or the embodiments of the asymmetric precursor RNA molecule as encoded by a polynucleotide, at least one linking RNA sequence (L) comprises an intron, preferably wherein all of the linking RNA sequences in the precursor RNA molecule comprise an intron. In an alternative embodiment, the precursor RNA molecule as encoded by a polynucleotide lacks an intron.

In embodiments of the first, second, third, fourth, or fifth aspects, or the embodiments of the asymmetric precursor RNA molecule, the loop sequence of a hairpin precursor RNA molecule, or either or both loops of a precursor ledRNA molecule, comprises a second copy of a sequence (seed sequence) from the dsRNA region, comprising either part or the whole of the sense or antisense sequence from the dsRNA region. For example, the precursor RNA molecule comprises a second sense or antisense sequence as the seed sequence which comprises at least 100 ribonucleotides from within the dsRNA region (seed region), incorporated into the loop sequence or elsewhere in the precursor RNA molecule. The loop sequence may be chimeric in comprising a seed sequence as well as other sequences related to the target RNA molecule or its complement. In an embodiment, the seed sequence is inserted into a region of the RNA molecule other than the loop, for example to 5′ or 3′ end of the precursor RNA molecule. In an embodiment, the duplex regions of the first and second components of the precursor ledRNA molecule targets different target RNA molecules and the first and second loop sequences correspond to regions from the different target transcripts.

In an embodiment, applicable to each of the embodiments of the first, second, third, fourth, or fifth aspects, or the embodiments of the asymmetric precursor RNA molecule, including the ledRNA structures, the double-stranded region(s) (B) in the precursor RNA molecule comprises bulges only as defined herein.

In an embodiment, applicable to each of the embodiments of the first, second, third, fourth, or fifth aspects, or the embodiments of the asymmetric precursor RNA molecule, including the precursor hairpin RNA and ledRNA molecules, the precursor RNA molecule further comprises a double-stranded region which comprises at least 23 contiguous basepairs and which lacks bulges. In an embodiment, between 5% and 40% of the at least 23 contiguous basepairs are G:U basepairs. Alternatively, the at least 23 contiguous basepairs are all canonical basepairs. The precursor RNA molecules (A) of these embodiments thereby provide a mixture of symmetrical and asymmetrical product RNA molecules (P). In an embodiment, when cleaved by a Dicer, the precursor RNA molecule provides more asymmetrical than symmetrical product RNA molecules (P).

In an embodiment, the double-stranded region which lacks bulges has a length of 30-200 contiguous basepairs, preferably at least 100 basepairs, more preferably 100-200, 100-300, 100-400, 100-500 or 100-600 basepairs in length.

In an embodiment, applicable to each of the embodiments of the first, second, third, fourth, or fifth aspects, or the embodiments of the asymmetric precursor RNA molecule, including the ledRNA structures, the one or more RNase(s) is a Type III ribonuclease, preferably a Dicer or Dicer-like (DCL) protein, more preferably a DCL2 and/or DCL4 protein or homologue. In an embodiment, the eukaryotic cell in which the precursor RNA is produced is a plant cell which is wild-type (unmodified) in gene(s) encoding DCL2 and/or DCL4 protein.

In an embodiment, applicable to each of the embodiments of the first, second, third, fourth, or fifth aspects, or the embodiments of the asymmetric precursor RNA molecule, including the precursor hairpin RNA and ledRNA molecules, one, two, three, four, five, or six, of the basepairs in at least some of the one or more double-stranded product RNA molecule(s) (P) are non-canonical basepairs, preferably G:U basepairs. Preferably, most of the double-stranded product RNA molecule(s) (P) produced from the precursor RNA molecule, independently comprise one, two, three, four, five, or six, G:U basepairs, for example at least 60%, at least 70%, at least 80%, or at least 90% of the product RNA molecules produced from the precursor RNA molecule comprise that number of G:U basepairs.

In an embodiment, applicable to each of the embodiments of the first, second, third, fourth, or fifth aspects, or the embodiments of the asymmetric precursor RNA molecule, including the ledRNA structures, on average every one in four to every one in six ribonucleotides in the dsRNA region (B) and/or the part (C) of the double-stranded RNA region and/or at least some of the product RNA molecules (P) form a non-canonical basepair or are not basepaired, preferably form a G:U basepair, and/or wherein between 5% and 40% of the ribonucleotides in the dsRNA region (B), in total, and/or the part (C) of the double-stranded RNA region, in total, and/or at least some of the product RNA molecules (P) form a non-canonical basepair or are not basepaired, preferably form a G:U basepair. Preferably, most of the double-stranded product RNA molecules (P) produced from the precursor RNA molecule, independently have on average about one-in-four to one-in-six basepairs that are G:U basepairs. Preferably, at least 60%, at least 70%, at least 80%, or at least 90% of the canonically-basepaired ribonucleotides in the dsRNA region, in total, are in subregions of 4-6 canonical basepairs. In an embodiment, more of the G:U basepairs of the population of the double-stranded product RNA molecules (P) produced from the precursor RNA molecule have the G ribonucleotide in a sense RNA sequence (H) and the U in an antisense RNA sequence (J).

In an embodiment, between 10% and 40%, between 10% and 35%, between 10% and 30%, or between 10% and 25% of the ribonucleotides in the dsRNA region (B), in total, and/or the part (C) of the double-stranded RNA region, in total, and/or in at least some of the product RNA molecules (P) form a G:U basepair. In an embodiment, between 15% and 40%, between 15% and 35%, between 15% and 30%, or between 15% and 25% of the ribonucleotides in the dsRNA region (B), in total, and/or the part (C) of the double-stranded RNA region, in total, and/or in at least some of the product RNA molecules (P) form a G:U basepair. In an embodiment, between 17% and 40%, between 17% and 35%, between 17% and 30%, or between 17% and 25% of the ribonucleotides in the dsRNA region (B), in total, and/or the part (C) of the double-stranded RNA region, in total, and/or in at least some of the product RNA molecules (P) form a G:U basepair. In an embodiment, about 10%, about 15%, about 20%, about 25%, about 30% or about 35% of the ribonucleotides in the dsRNA region (B), in total, and/or the part (C) of the double-stranded RNA region, in total, and/or in at least some of the product RNA molecules (P) form a G:U basepair.

In an embodiment, applicable to each of the embodiments of the first, second, third, fourth, or fifth aspects, or the embodiments of the asymmetric precursor RNA molecule, including the precursor hairpin RNA and ledRNA molecules, the at least one double-stranded RNA region (B), preferably all of the double-stranded RNA regions (B), in the precursor RNA molecule lacks 8 contiguous canonical basepairs. This can readily be achieved by the introduction of suitably distributed G:U basepairs in addition to bulges in the double-stranded RNA regions.

In an embodiment, applicable to each of the embodiments of the first, second, third, fourth, or fifth aspects, or the embodiments of the asymmetric precursor RNA molecule, including the precursor hairpin RNA and ledRNA molecules, the precursor RNA molecule, following cleavage by the one or more RNAses, produces at least two, at least three, at least four, at least five, at least six, at least seven, at least eight, at least nine, at least 10, at least 15, at least 20, at least 30, at least 40, at least 50, or at least 100 different product RNA molecules (P). In an embodiment, the precursor RNA molecule, following cleavage by the one or more RNAses, produces at least two, at least three, at least four, at least five, at least six, at least seven, at least eight, at least nine, at least 10, at least 11, at least 12, at least 13, at least 14, at least 15, at least 20, at least 25, at least 30, at least 40, up to about 50 non-overlapping different product RNA molecules (P).

In an embodiment, applicable to each of the embodiments of the first, second, third, fourth, or fifth aspects, or the embodiments of the asymmetric precursor RNA molecule, including the precursor hairpin RNA and ledRNA molecules, the first RNA strand (D) and/or the second RNA strand (F), preferably both, comprise at least 50, at least 100, at least 200, at least 300, at least 400, at least 500, at least 600, at least 700, at least 800, at least 900, at least 1,000, or 100 to 1,000, or 50 to 1000 nucleotides, or 50 to 500, or 100 to 500 ribonucleotides. In a preferred embodiment, the first RNA strand (D) is shorter than the second RNA strand (F).

In an embodiment, applicable to each of the embodiments of the first, second, third, fourth, or fifth aspects, or the embodiments of the asymmetric precursor RNA molecule, including the precursor hairpin RNA and ledRNA molecules, the precursor RNA molecule has a single double-stranded RNA region (B) and a single part (C) which extends to encompass the whole double-stranded RNA region (B), i.e. the single part (C) corresponds to the entire double-stranded RNA region (B).

In an embodiment, both RNA strands (D) and (F) of the double-stranded region (B) are 23 to 33 ribonucleotides in length, and the double-stranded region (B) comprises at least 18 basepairs, preferably at least 19 or at least 20 basepairs. This is preferred where the eukaryotic cell is a vertebrate animal cell, particularly a mammalian cell. In this embodiment, preferably one, two, three, four, five, or six of the basepairs in at least some of the one or more double-stranded product RNA molecule(s) (P) produced from the precursor RNA molecule are non-canonical basepairs, preferably G:U basepairs.

In an embodiment, applicable to each of the embodiments of the first, second and third aspects, including the ledRNA molecules,
(i) RNA strands (D) and (F) of the double-stranded region (B) are each 34 to 200 ribonucleotides in length,
(ii) at least 80%, preferably at least 90%, of the ribonucleotides in the RNA strands (D) and (F), in total, are basepaired, thereby forming the double-stranded region (B) of the precursor RNA molecule, and
(iii) between 5% and 40% of the ribonucleotides in the dsRNA region (B), in total, are basepaired in a non-canonical basepair or are not basepaired, preferably basepaired in a G:U basepair. In this context, preferably between 10% and 40%, between 10% and 35%, between 10% and 30%, or between 10% and 25% of the ribonucleotides in the dsRNA region (B), in total, and/or the part (C) of the double-stranded RNA region, in total, and/or in at least some of the product RNA molecules (P) form a G:U basepair. More preferably, between 15% and 40%, between 15% and 35%, between 15% and 30%, or between 15% and 25% of the ribonucleotides in the dsRNA region (B), in total, and/or the part (C) of the double-stranded RNA region, in total, and/or in at least some of the product RNA molecules (P) form a G:U basepair. Even more preferably, between 17% and 40%, between 17% and 35%, between 17% and 30%, or between 17% and 25% of the ribonucleotides in the dsRNA region (B), in total, and/or the part (C) of the double-stranded RNA region, in total, and/or in at least some of the product RNA molecules (P) form a G:U basepair. In an embodiment, about 10%, about 15%, about 20%, about 25%, about 30% or about 35% of the ribonucleotides in the dsRNA region (B), in total, and/or the part (C) of the double-stranded RNA region, in total, and/or in at least some of the product RNA molecules (P) form a G:U basepair. Such molecules are particularly useful in vertebrate animal cells such as mammalian animal cells.

In an embodiment, applicable to each of the embodiments of the first, second, third, fourth, or fifth aspects, or the embodiments of the asymmetric precursor RNA molecule, including the ledRNA molecules, the antisense RNA sequence (J) from at least one of the product RNA molecules (P) produced from the precursor RNA molecule, preferably multiple, different product RNA molecules, are capable of hybridising to a region (R) of a target RNA molecule in a eukaryotic cell through all of at least ribonucleotides 2 to 8 of the antisense RNA sequence (J) basepairing with ribonucleotides within the region (R) of the target RNA molecule, preferably all of at least ribonucleotides 2-10 or 2-11 of the antisense RNA sequence (J).

In an embodiment, at least 50%, at least 75%, at least 90%, or at least 95%, of the ribonucleotides of the antisense RNA sequence (J) are capable of basepairing to ribonucleotides within the region (R) of the target RNA molecule in the eukaryotic cell.

In an embodiment, all of the ribonucleotides of the antisense RNA sequence (J), preferably all of the ribonucleotides of multiple, different antisense RNA sequences (J), are capable of basepairing to ribonucleotides in a region (R) of a target RNA molecule in the eukaryotic cell. In a preferred embodiment, all of the ribonucleotides of the antisense RNA sequence (J), preferably all of the ribonucleotides of multiple, different antisense RNA sequences (J), are capable of basepairing by canonical basepairs to ribonucleotides in the region (R) of the target RNA molecule in the eukaryotic cell, particularly for a plant cell, a fungal cell, a nematode cell, or an arthropod cell such as an insect cell.

In an embodiment, applicable to each of the embodiments of the first, second and third aspects, or the embodiments of the asymmetric precursor RNA molecule, including the precursor hairpin RNA and ledRNA molecules, the eukaryotic cell comprising the target RNA molecule is a vertebrate animal cell, preferably a mammalian cell, more preferably a human cell, or a non-mammalian vertebrate animal cell such as a bird cell or fish cell. The vertebrate animal cell may be of a companion animal or a livestock animal.

In an embodiment, applicable to each of the embodiments of the first, second, third, fourth, or fifth aspects, or the embodiments of the asymmetric precursor RNA molecule, including the precursor hairpin RNA and ledRNA molecules,
(i) the first RNA sequence (E) differs from a corresponding wild-type RNA sequence in the target RNA molecule by deletion of one or more ribonucleotides from the corresponding wild-type RNA sequence to make the first RNA sequence, preferably by deletion of multiple ribonucleotides in the Δ22, Δ23 or Δ24 pattern, and/or
(ii) the second RNA sequence (G) differs from a fully complementary sequence to the corresponding wild-type RNA sequence in the target RNA molecule by insertion of one or more ribonucleotides into the fully complementary sequence to make the second RNA sequence, preferably (i). In a preferred embodiment, the second RNA strand (F), along its full length, is identical in sequence to the complement of the wild-type RNA sequence in the target RNA molecule, particularly for a target RNA molecule in a plant, arthropod such as insect, arachnid, or decapod, nematode or fungal cell.

In an embodiment, applicable to each of the embodiments of the first, second, third, fourth, or fifth aspects, or the embodiments of the asymmetric precursor RNA molecule, including the precursor hairpin RNA and ledRNA molecules, the deletion of ribonucleotides from the corresponding wild-type RNA sequence occurs at one or more or all of the ribonucleotide positions corresponding to the non-basepaired ribonucleotides in the second RNA sequence (G) as defined herein.

In an embodiment, applicable to each of the embodiments of the first, second, third, fourth, or fifth aspects, including the precursor hairpin RNA and ledRNA molecules, the second RNA sequence (G) is identical in length to the fully complementary sequence to the corresponding wild-type RNA sequence in the target RNA molecule. That is, the second RNA sequence (G) does not have insertions or deletions of ribonucleotides relative to the complement of the region of the target RNA molecule.

In an embodiment, applicable to each of the embodiments of the first, second, third, fourth, or fifth aspects, or the embodiments of the asymmetric precursor RNA molecule, including the precursor hairpin RNA and ledRNA molecules,
(i) the first RNA sequence (E) differs from the corresponding wild-type RNA sequence in the target RNA molecule by substitution of one or more A or C ribonucleotides in the corresponding wild-type RNA sequence to G or U ribonucleotides, respectively, thereby resulting in the first RNA sequence, wherein the G or U ribonucleotides are involved in G:U basepairs with ribonucleotides of the second RNA sequence (G), and/or
(ii) the second RNA sequence (G) differs from the fully complementary sequence by substitution of one or more A or C ribonucleotides in the fully complementary sequence to G or U ribonucleotides, thereby resulting in the second RNA sequence, wherein the G or U ribonucleotides are involved in G:U basepairs with ribonucleotides of the first RNA sequence (E), preferably (i).

In a further aspect, the present invention provides a precursor RNA molecule comprising;
i) a first double-stranded RNA region (B) of a first aspect of the invention, and a second double-stranded RNA region (B) of a second aspect of the invention,
ii) a first double-stranded RNA region (B) of a first aspect of the invention, and a second double-stranded RNA region (B) of a third aspect of the invention,
iii) a first double-stranded RNA region (B) of a second aspect of the invention, and a second double-stranded RNA region (B) of a third aspect of the invention,
iv) a first double-stranded RNA region (B) of a first aspect of the invention, a second double-stranded RNA region (B) of a second aspect of the invention, and a third double-stranded RNA region (B) of a third aspect of the invention,
v) a first double-stranded RNA region (B) of a first aspect of the invention, and a second double-stranded RNA region (B) of a first aspect of the invention,
vi) a first double-stranded RNA region (B) of a second aspect of the invention, and a second double-stranded RNA region (B) of a second aspect of the invention,
vii) a first double-stranded RNA region (B) of a third aspect of the invention, and a second double-stranded RNA region (B) of a third aspect of the invention, or
viii) any combination of i) to vii). All combinations of the embodiments of the first, second and third aspects are contemplated here.

In an embodiment, the precursor RNA molecule comprises two, three, four, five, 10, 15, 20 or more double-stranded RNA regions of a first aspect of the invention, two, three, four, five, 10, 15, 20 or more double-stranded RNA regions of a second aspect of the invention, two, three, four, five, 10, 15, 20 or more double-stranded RNA regions of a third aspect of the invention, or any combination thereof.

Each of the embodiments of the precursor RNA molecule of the first, second, third, fourth and fifth aspects as described above, and the precursor RNA molecule lacking a linking RNA sequence, are useful for reducing expression and/or activity of a target RNA molecule in a eukaryotic cell, preferably a plant cell, fungal cell or nematode cell, or an arthropod cell such as an insect cell or a decapod cell. They are also useful in reducing expression and/or activity of a viral target RNA molecule, such as for a plant virus, including the specific plant viruses mentioned herein. They are also useful for reducing expression and/or activity of a target RNA molecule in other invertebrate animal cells such as an arthropod cell or insect cell, nematode, or in a non-mammalian vertebrate animal cell. Single or combinations of precursor RNA molecules are also useful in reducing expression and/or activity of multiple target RNA molecules, for example produced from multiple genes. The precursor RNA molecules are useful by way of the processing that produces the product RNA molecules (P), wherein the antisense sequences (J) function with an Argonaute protein in a RISC, the mechanism well known in the art. They may also be useful through enhanced production of secondary antisense sRNA molecules relative to the corresponding conventional RNA molecule. The precursor RNA molecule may be produced in a plant cell to reduce an insect target RNA molecule upon ingestion, or a fungal pathogen or nematode target RNA molecule, or applied topically to a plant or insect to reduce a target RNA molecule.

In an aspect, the present invention provides a double-stranded RNA molecule (P) consisting of a sense RNA sequence (H) of 21 contiguous ribonucleotides and an antisense RNA sequence (J) of 22 contiguous ribonucleotides and one or two or three bulges,
wherein 18 or 19 of ribonucleotides 1 to 19 of the sense RNA sequence (H) each basepair with a ribonucleotide of ribonucleotides 1 to 20 of the antisense RNA sequence (J),
wherein each of the one or two or three bulges is immediately flanked by ribonucleotides which are basepaired in the double-stranded RNA molecule,
wherein ribonucleotides 20 and 21 of the sense RNA sequence form a 2-ribonucleotide unpaired 3′ overhang and ribonucleotides 21 and 22 of the antisense RNA sequence form a 2-ribonucleotide unpaired 3′ overhang,
wherein ribonucleotides 1 and 2 of the sense RNA sequence basepair with ribonucleotides 19 and 20, respectively, of the antisense RNA sequence, and
wherein ribonucleotides 18 and 19 of the sense RNA sequence basepair with ribonucleotides 1 and 2, respectively, of the antisense RNA sequence.

In an embodiment,
(i) ribonucleotides 1, 2 and 3 of the sense RNA sequence (H) basepair with ribonucleotides 18, 19 and 20, respectively, of the antisense RNA sequence (J), or
(ii) ribonucleotides 17, 18 and 19 of the sense RNA sequence (H) basepair with ribonucleotides 1, 2 and 3, respectively, of the antisense RNA sequence (J), or preferably both (i) and (ii). In further features of these embodiments, the product RNA molecules may have at least four, at least five or at least six contiguous basepairs at one or both ends of the double-stranded molecule before any bulge. In these embodiments, any of those basepairs may be G:U basepairs, preferably one or more of ribonucleotides 1, 2 and 3 of the antisense sequence (J) are involved in G:U basepairs, more preferably one or both of ribonucleotides 1 and 2 of the antisense sequence (J) are involved in G:U basepairs, even more preferably ribonucleotide 1 of the antisense sequence (J) is involved in G:U basepairs. Most preferably, one or more of ribonucleotides 1, 2 and 3 of the antisense sequence (J) are U ribonucleotides, or one or both of ribonucleotides 1 and 2 are U ribonucleotides, or ribonucleotide 1 is a U ribonucleotide.

In an aspect, the present invention provides a double-stranded RNA molecule (P) consisting of a sense RNA sequence (H) of 21 contiguous ribonucleotides and an antisense RNA sequence (J) of 23 contiguous ribonucleotides and one or two or three or four bulges,
wherein 18 or 19 of ribonucleotides 1 to 19 of the sense RNA sequence (H) each basepair with a ribonucleotide of ribonucleotides 1 to 21 of the antisense RNA sequence (J),
wherein each of the one or two or three or four bulges is immediately flanked by ribonucleotides which are basepaired in the double-stranded RNA molecule,
wherein ribonucleotides 20 and 21 of the sense RNA sequence form a 2-ribonucleotide unpaired 3′ overhang and ribonucleotides 22 and 23 of the antisense RNA sequence form a 2-ribonucleotide unpaired 3′ overhang,
wherein ribonucleotides 1 and 2 of the sense RNA sequence basepair with ribonucleotides 20 and 21, respectively, of the antisense RNA sequence, and
wherein ribonucleotides 18 and 19 of the sense RNA sequence basepair with ribonucleotides 1 and 2, respectively, of the antisense RNA sequence.

In an embodiment,
(i) ribonucleotides 1, 2 and 3 of the sense RNA sequence (H) basepair with ribonucleotides 19, 20 and 21, respectively, of the antisense RNA sequence (J), or
(ii) ribonucleotides 17, 18 and 19 of the sense RNA sequence (H) basepair with ribonucleotides 1, 2 and 3, respectively, of the antisense RNA sequence (J),
or preferably both (i) and (ii). In further features of these embodiments, the product RNA molecules may have at least four, at least five or at least six contiguous basepairs at one or both ends of the double-stranded molecule before any bulge. In these embodiments, any of those basepairs may be G:U basepairs, preferably one or more of ribonucleotides 1, 2 and 3 of the antisense sequence (J) are involved in G:U basepairs, more preferably one or both of ribonucleotides 1 and 2 of the antisense sequence (J) are involved in G:U basepairs, even more preferably ribonucleotide 1 of the antisense sequence (J) is involved in G:U basepairs. Most preferably, one or more of ribonucleotides 1, 2 and 3 of the antisense sequence (J) are U ribonucleotides, or one or both of ribonucleotides 1 and 2 are U ribonucleotides, or ribonucleotide 1 is a U ribonucleotide.

In an aspect, the present invention provides a double-stranded RNA molecule (P) consisting of a sense RNA sequence (H) of 21 contiguous ribonucleotides and an antisense RNA sequence (J) of 24 contiguous ribonucleotides and one or two or three or four or five bulges,
wherein 18 or 19 of ribonucleotides 1 to 19 of the sense RNA sequence each basepair with a ribonucleotide of ribonucleotides 1 to 22 of the antisense RNA sequence (J),
wherein each of the one or two or three or four or five bulges is immediately flanked by ribonucleotides which are basepaired in the double-stranded RNA molecule,
wherein ribonucleotides 20 and 21 of the sense RNA sequence form a 2-ribonucleotide unpaired 3′ overhang and ribonucleotides 23 and 24 of the antisense RNA sequence form a 2-ribonucleotide unpaired 3′ overhang,
wherein ribonucleotides 1 and 2 of the sense RNA sequence basepair with ribonucleotides 21 and 22, respectively, of the antisense RNA sequence, and
wherein ribonucleotides 18 and 19 of the sense RNA sequence basepair with ribonucleotides 1 and 2, respectively, of the antisense RNA sequence.

In an embodiment,
(i) ribonucleotides 1, 2 and 3 of the sense RNA sequence (H) basepair with ribonucleotides 20, 21 and 22, respectively, of the antisense RNA sequence (J), or
(ii) ribonucleotides 17, 18 and 19 of the sense RNA sequence (H) basepair with ribonucleotides 1, 2 and 3, respectively, of the antisense RNA sequence (J),
or preferably both (i) and (ii). In further features of these embodiments, the product RNA molecules may have at least four, at least five or at least six contiguous basepairs at one or both ends of the double-stranded molecule before any bulge. In these embodiments, any of those basepairs may be G:U basepairs, preferably one or more of ribonucleotides 1, 2 and 3 of the antisense sequence (J) are involved in G:U basepairs, more preferably one or both of ribonucleotides 1 and 2 of the antisense sequence (J) are involved in G:U basepairs, even more preferably ribonucleotide 1 of the antisense sequence (J) is involved in G:U basepairs. Most preferably, one or more of ribonucleotides 1, 2 and 3 of the antisense sequence (J) are U ribonucleotides, or one or both of ribonucleotides 1 and 2 are U ribonucleotides, or ribonucleotide 1 is a U ribonucleotide.

In an embodiment, applicable to each of these aspects, the basepairing between ribonucleotides of the sense RNA sequence (H) and ribonucleotides of the antisense RNA sequence (J) comprises one, two, three, four, five or six G:U basepairs. Preferably, all of the G:U basepairs have the G ribonucleotide in the sense RNA sequence (H) and the U ribonucleotide in the antisense RNA sequence (J).

In an embodiment, applicable to each of these aspects, the basepairing between ribonucleotides of the sense RNA sequence (H) and ribonucleotides of the antisense RNA sequence (J) lacks G:U basepairs, preferably lacks non-canonical basepairs.

In an embodiment, applicable to all of the above aspects, ribonucleotide 1 of the antisense RNA sequence (J) in at least some of the product RNA molecules (P) is a U ribonucleotide. In an embodiment, ribonucleotide 1 of the antisense RNA sequence (J) in at least some of the product RNA molecules (P) is a U ribonucleotide which is basepaired to a G ribonucleotide in the sense RNA sequence (H).

In an embodiment, applicable to all of the above aspects, all of at least ribonucleotides 2-8, or 2-10 or 2-11, of the antisense RNA sequence (J) basepair to ribonucleotides in a region (R) of a target RNA molecule in a eukaryotic cell, preferably all of the ribonucleotides of the antisense RNA sequence basepair to ribonucleotides in the region (R) of the target RNA molecule. In a preferred embodiment, all of the ribonucleotides of the antisense RNA sequence (J) basepair to ribonucleotides in the region (R) of the target RNA molecule by canonical basepairs.

In an embodiment of the fourth or fifth aspects or the embodiments of the asymmetric precursor RNA molecule of the first, second, third, fourth or fifth aspects, the double-stranded RNA molecule consists of a sense RNA sequence of 21 contiguous ribonucleotides and an antisense RNA sequence of 22 contiguous ribonucleotides and one or two or three bulges,
wherein 18 or 19 of ribonucleotides 1 to 19 of the sense RNA sequence each basepair with a ribonucleotide of ribonucleotides 1 to 20 of the antisense RNA sequence,
wherein the basepairs between the sense RNA sequence and the antisense RNA sequence in each of the double-stranded product RNA molecules comprise, independently, 2, 3, 4, 5, or 6 G:U basepairs,
wherein each of the one or two or three bulges is immediately flanked by ribonucleotides which are basepaired in the double-stranded RNA molecule,
wherein ribonucleotides 20 and 21 of the sense RNA sequence form a 2-ribonucleotide unpaired 3′ overhang and ribonucleotides 21 and 22 of the antisense RNA sequence form a 2-ribonucleotide unpaired 3′ overhang,
wherein ribonucleotides 1 and 2 of the sense RNA sequence basepair with ribonucleotides 19 and 20, respectively, of the antisense RNA sequence, and
wherein ribonucleotides 18 and 19 of the sense RNA sequence basepair with ribonucleotides 1 and 2, respectively, of the antisense RNA sequence.

In an embodiment, (i) ribonucleotides 1, 2, and 3 of the sense RNA sequence basepair with ribonucleotides 18, 19 and 20, respectively, of the antisense RNA sequence, or (ii) ribonucleotides 17, 18, and 19 of the sense RNA sequence basepair with ribonucleotides 1, 2, and 3, respectively, of the antisense RNA sequence, or preferably both (i) and (ii).

In an embodiment of the fourth or fifth aspects or the embodiments of the asymmetric precursor RNA molecule of the first, second, third, fourth, or fifth aspects, the double-stranded RNA molecule consists of a sense RNA sequence of 21 contiguous ribonucleotides and an antisense RNA sequence of 23 contiguous ribonucleotides and one or two or three or four bulges,
wherein 18 or 19 of ribonucleotides 1 to 19 of the sense RNA sequence each basepair with a ribonucleotide of ribonucleotides 1 to 21 of the antisense RNA sequence,
wherein the basepairs between the sense RNA sequence and the antisense RNA sequence in each of the double-stranded product RNA molecules comprise, independently, 2, 3, 4, 5, or 6 G:U basepairs,
wherein each of the one or two or three or four bulges is immediately flanked by ribonucleotides which are basepaired in the double-stranded RNA molecule,
wherein ribonucleotides 20 and 21 of the sense RNA sequence form a 2-ribonucleotide unpaired 3′ overhang and ribonucleotides 22 and 23 of the antisense RNA sequence form a 2-ribonucleotide unpaired 3′ overhang,
wherein ribonucleotides 1 and 2 of the sense RNA sequence basepair with ribonucleotides 20 and 21, respectively, of the antisense RNA sequence, and
wherein ribonucleotides 18 and 19 of the sense RNA sequence basepair with ribonucleotides 1 and 2, respectively, of the antisense RNA sequence.

In an embodiment, (i) ribonucleotides 1, 2, and 3 of the sense RNA sequence basepair with ribonucleotides 19, 20, and 21, respectively, of the antisense RNA sequence, or (ii) ribonucleotides 17, 18, and 19 of the sense RNA sequence basepair with ribonucleotides 1, 2 and 3, respectively, of the antisense RNA sequence, or preferably both (i) and (ii).

In an embodiment of the fourth or fifth aspects or the embodiments of the asymmetric precursor RNA molecule of the first, second, third, fourth, or fifth aspects, the double-stranded RNA molecule consists of a sense RNA sequence of 21 contiguous ribonucleotides and an antisense RNA sequence of 24 contiguous ribonucleotides and one or two or three or four or five bulges,
wherein 18 or 19 of ribonucleotides 1 to 19 of the sense RNA sequence each basepair with a ribonucleotide of ribonucleotides 1 to 22 of the antisense RNA sequence,
wherein the basepairs between the sense RNA sequence and the antisense RNA sequence in each of the double-stranded product RNA molecules comprise, independently, 2, 3, 4, 5, or 6 G:U basepairs,
wherein each of the one or two or three or four or five bulges is immediately flanked by ribonucleotides which are basepaired in the double-stranded RNA molecule,
wherein ribonucleotides 20 and 21 of the sense RNA sequence form a 2-ribonucleotide unpaired 3′ overhang and ribonucleotides 23 and 24 of the antisense RNA sequence form a 2-ribonucleotide unpaired 3′ overhang,
wherein ribonucleotides 1 and 2 of the sense RNA sequence basepair with ribonucleotides 21 and 22, respectively, of the antisense RNA sequence, and
wherein ribonucleotides 18 and 19 of the sense RNA sequence basepair with ribonucleotides 1 and 2, respectively, of the antisense RNA sequence.

In an embodiment, (i) ribonucleotides 1, 2 and 3 of the sense RNA sequence basepair with ribonucleotides 20, 21, and 22, respectively, of the antisense RNA sequence, or (ii) ribonucleotides 17, 18, and 19 of the sense RNA sequence basepair with ribonucleotides 1, 2, and 3, respectively, of the antisense RNA sequence, or preferably both (i) and (ii).

In an embodiment, ribonucleotide 1 of the antisense RNA sequence is a U ribonucleotide. In an embodiment, ribonucleotide 1 of the antisense RNA sequence is a U ribonucleotide which is basepaired to a G ribonucleotide in the sense RNA sequence.

In an embodiment, the double-stranded RNA molecule (P) has one or more of any of the features defined herein.

As exemplified herein, it is possible to change the numbers of siRNA molecules in a eukaryotic cell comprising an antisense RNA sequence (J) of 22, 23 or 24 ribonucleotides relative to 21 ribonucleotides by the Δ22, Δ23 or Δ24 modifications in a precursor RNA molecule, relative to a corresponding conventional precursor RNA molecule without the modifications, especially along the full length of a double-stranded region (B) comprising a second RNA strand (F) of at least 100 ribonucleotides in length. Therefore, in an aspect, the present invention provides a population of multiple, different double-stranded RNA molecules (P) of the invention. In another aspect, the present invention provides a population of multiple, different double-stranded RNA molecules comprising double-stranded RNA molecules of the invention, or any combination thereof.

In an embodiment, the population of multiple, different double-stranded RNA molecules (P) comprises at least two, at least three, at least four, at least five, at least six, at least seven, at least eight, at least nine, at least 10, at least 15, at least 20, at least 30, at least 40, at least 50, or at least 100 different product RNA molecules (P) which each independently comprise:
(i) an antisense RNA sequence (J) of 22 contiguous ribonucleotides and one or two or three bulges,
(ii) an antisense RNA sequence (J) of 23 contiguous ribonucleotides and one or two or three or four bulges,
(iii) an antisense RNA sequence (J) of 24 contiguous ribonucleotides and one or two or three or four or five bulges, or
(iv) any combination of (i), (ii) and (iii).

In an embodiment, the population of multiple, different double-stranded RNA molecules (P) comprises (i) or (ii) above.

In an embodiment,
(i) more antisense RNA sequences (J) in the population consist of 22 ribonucleotides than consist of 21 ribonucleotides,
(ii) more antisense RNA sequences (J) in the population consist of 23 ribonucleotides than consist of 21 ribonucleotides,
(iii) more antisense RNA sequences (J) in the population consist of 24 ribonucleotides than consist of 21 ribonucleotides, or
(iv) any combination of (i), (ii) and (iii).

In an embodiment of the first, second, third, fourth or fifth aspects or the embodiments of the asymmetric precursor RNA molecule, the present invention provides a population of multiple, different double-stranded RNA molecules of the invention. In an embodiment, the population of multiple, different double-stranded RNA molecules comprises at least two, at least three, at least four, at least five, at least six, at least seven, at least eight, at least nine, at least 10, at least 15, at least 20, at least 30, at least 40, at least 50, or at least 100 different product RNA molecules which each independently comprise:
(i) an antisense RNA sequence of 22 contiguous ribonucleotides and one or two or three bulges,
(ii) an antisense RNA sequence of 23 contiguous ribonucleotides and one or two or three or four bulges,
(iii) an antisense RNA sequence of 24 contiguous ribonucleotides and one or two or three or four or five bulges, or
(iv) any combination of (i), (ii) and (iii).

In an embodiment, the population of multiple, different double-stranded RNA molecules comprise the following features:
(i) more antisense RNA sequences in the population consist of 22 ribonucleotides than consist of 21 ribonucleotides,
(ii) more antisense RNA sequences in the population consist of 23 ribonucleotides than consist of 21 ribonucleotides,
(iii) more antisense RNA sequences in the population consist of 24 ribonucleotides than consist of 21 ribonucleotides, or
(iv) any combination of (i), (ii) and (iii).

In an embodiment of the first, second, third, fourth or fifth aspects or the embodiments of the asymmetric precursor RNA molecule, the invention provides a method of increasing the amount of small interfering RNAs (sRNAs) of 22, 23 or 24 ribonucleotides in length in an insect cell or insect, or a fungal cell or fungus, or of increasing the ratio of the amount of sRNAs of 22, 23 or 24 ribonucleotides relative to the amount of sRNAs of 21 ribonucleotides in length, the method comprising delivering to the eukaryotic cell such as a plant cell, nematode cell, insect cell or fungal cell, insect, or fungus one or more or all of the precursor RNA molecule, the double-stranded RNA molecule, the population of multiple, different double-stranded RNA molecules, the polynucleotide, the vector, the cell, the non-human organism or part thereof, the extract, and the composition of the invention.

In an aspect, the present invention provides a eukaryotic cell comprising the double-stranded RNA molecule (P) of the invention.

In an aspect, the present invention provides a eukaryotic cell comprising the population of multiple, different double-stranded RNA molecules (P) of the invention.

In an embodiment of the first, second, third, fourth or fifth aspects or the embodiments of the asymmetric precursor RNA molecule, the present invention provides a eukaryotic cell such as a plant cell, nematode cell, insect cell or fungal cell, comprising one or more or all of the precursor RNA molecules, the double-stranded RNA molecules, and the population of multiple, different double-stranded RNA molecules of the invention, wherein the eukaryotic cell is preferably a plant cell, insect cell, fungal cell such as a yeast cell.

In an embodiment, the eukaryotic cell comprises one or both of the precursor RNA molecule, and the population of multiple, different double-stranded RNA molecules of the invention.

In an aspect, the present invention provides a double-stranded RNA molecule (P) obtainable by, or obtained from, the cleavage of the precursor RNA molecule of the invention, wherein the double-stranded RNA molecule has one or more of any of the features defined herein.

In an aspect, the present invention provides a double-stranded RNA molecule (P) obtainable by, or obtained from, the cleavage of the precursor RNA molecule of the invention.

In an aspect, the present invention provides a method of identifying a double-stranded RNA molecule (P), or a precursor RNA molecule (A), for reducing the amount and/or activity of a target RNA molecule of interest, the method comprising:
i) producing a precursor RNA molecule (A) and/or a double-stranded RNA molecule of the invention, or a population of multiple, different precursor RNA molecules (A) and/or multiple, different double-stranded RNA molecules of the invention,
ii) determining the ability of the precursor RNA molecule or double-stranded RNA molecule, or members of the population of multiple, different precursor RNA molecules or multiple, different double-stranded RNA molecules, to reduce the amount and/or activity of a target RNA molecule of interest. The RNA molecules of the invention can thereby by used in screening assays to identify a suitable precursor RNA molecule that down-regulates a known target RNA molecule, or to identify a target RNA molecule associated with a suitable phenotype or function in the eukaryotic cell.

In an embodiment, step i) comprises expressing a precursor RNA molecule (A) of the invention in a eukaryotic cell, wherein the precursor RNA molecule is cleaved in the eukaryotic cell by one or more ribonucleases (RNases) to produce the double-stranded RNA molecule or the population of multiple, different double-stranded RNA molecules.

In an embodiment, the method further comprises designing the double-stranded RNA molecule based on a region (R) of the ribonucleotide sequence of the target RNA molecule of interest, preferably using the features defined herein.

In an embodiment, the method further comprises a step of producing more of the double-stranded RNA molecule or the precursor RNA molecule after step ii), for example in commercial quantities of for a kit comprising the double-stranded RNA molecule or the precursor RNA molecule.

In an embodiment for the precursor RNA molecules of the fourth or fifth aspects or the embodiments of the asymmetric precursor RNA molecule, the invention provides a method of identifying a double-stranded RNA molecule, or a precursor RNA molecule, for reducing the amount and/or activity of a target RNA molecule of interest in an insect cell or a fungal cell, the method comprising:
i) producing a precursor RNA molecule, and/or a double-stranded RNA molecule, or a population of multiple, different precursor RNA molecules of the invention, and
ii) determining the ability of the precursor RNA molecule or double-stranded RNA molecule, or members of the population of multiple, different precursor RNA molecules, or the population of multiple, different double-stranded RNA molecules, to reduce the amount and/or activity of the target RNA molecule of interest in the insect or fungal cell.

In an embodiment, step i) comprises introducing a precursor RNA molecule of the invention into: (i) an insect cell, preferably by ingestion, soaking, dusting, spraying or injection of an insect comprising the insect cell; and/or (ii) a fungal cell, preferably by topical application such as soaking, dusting, spraying or applying a composition comprising the precursor RNA molecule to the fungal cell; wherein the precursor RNA molecule is cleaved in the insect cell or fungal cell by a Dicer to produce the double-stranded RNA molecule or the population of multiple, different double-stranded RNA molecules.

In an embodiment, step i) comprises introducing a precursor RNA molecule of the invention into a plant cell or microbial cell such as a bacterial cell or yeast cell, and step (ii) comprises delivering the plant cell or microbial cell of step (i) to the insect cell or the fungal cell, preferably by ingestion of the plant cell or microbial cell, wherein the precursor RNA molecule is cleaved in the insect cell or fungal cell by a Dicer to produce the double-stranded RNA molecule or the population of multiple, different double-stranded RNA molecules. In an embodiment, the method further comprises a step of producing more of the double-stranded RNA molecule or the precursor RNA molecule after step ii).

Also provided is a double-stranded RNA molecule (P) or a precursor RNA molecule (A) identified or produced using a method of the invention.

In an aspect, the present invention provides an isolated and/or exogenous polynucleotide encoding the precursor RNA molecule of the invention.

In an embodiment, the polynucleotide is a DNA construct such as a chimeric DNA construct. Alternatively, the polynucleotide is a RNA construct, for example a RNA construct based on a viral RNA genome.

In an embodiment, the polynucleotide is operably linked to a promoter capable of directing expression of the precursor RNA molecule in a host cell, preferably a eukaryotic cell such as a plant cell, and optionally a polyadenylation region/transcription terminator or a transcription termination sequence. In an embodiment, the promoter is heterologous to the target RNA molecule.

In an embodiment, the promoter is as an RNA polymerase III promoter, an RNA polymerase II promoter, or a promoter which functions in an in vitro transcription reaction.

In an aspect, the present invention provides a vector comprising a polynucleotide of the invention.

In an embodiment, the vector is a viral vector, such as a DNA viral vector or an RNA viral vector or a plasmid.

In an aspect, the present invention provides a host cell comprising one or more or all of the precursor RNA molecule of the invention, the double-stranded RNA molecule of the invention, the population of multiple, different double-stranded RNA molecules of the invention, the polynucleotide of the invention, or the vector of the invention.

In an embodiment, the host cell comprises a population of two, three, four, five, 10, 15, 20, 50, 100 or more different, double-stranded RNA molecules of the invention.

In an embodiment, the host cell is a eukaryotic cell. In an embodiment, the host cell is a micro-organism, for example a bacterial cell such as an E. coli cell, or a yeast cell such as, for example, Saccharomyces cerevisiae. In an embodiment, the micro-organism has been modified to reduce catabolism of double-stranded RNA molecules or to enhance their accumulation. In an embodiment, the host cell is a non-human host cell, or a cell in cell culture or in vitro, or a cell in a non-human organism.

In a further aspect, the present invention provides eukaryotic cell comprising one or more or all of a precursor RNA molecule of the invention, a polynucleotide encoding the precursor RNA molecule, the double-stranded RNA molecule (P) of the invention, and the population of multiple, different double-stranded RNA molecules (P) of the invention, preferably wherein the cell is a non-human cell or a eukaryotic cell in vitro.

In an embodiment, the host cell is a plant cell, a fungal cell, or an animal cell, preferably a plant cell, an arthropod cell such as an insect, arachnid, or decapod cell, a nematode cell or a fungal cell. In an embodiment, the plant cell is wild-type for a gene encoding a DCL4 protein and/or a gene encoding a DCL2 protein. In an embodiment, the plant cell is other than a Nicotiana benthamiana or Nicotiana tabacum cell. In an embodiment, the host cell is a fungal cell or an animal cell which is wild-type for Dicer protein(s).

In an embodiment, applicable to all of the embodiments of the above aspects, the double-stranded RNA molecule and/or the precursor RNA molecule and/or the population of multiple, different double-stranded RNA molecules do not naturally occur in the cell.

In an embodiment, applicable to all of the embodiments of the above aspects, the host cell is dead and/or incapable of reproduction. For example, the dead or inactivated host cell is a microbial cell. Alternatively, the dead or inactivated cell is a plant cell, or of an insect pest.

In an aspect, the present invention provides a non-human organism, or a part thereof, comprising a cell of the invention.

In an embodiment, the non-human organism or part thereof is a transgenic non-human organism or part thereof, being transgenic for a polynucleotide of the invention, preferably a transgenic plant or part thereof.

In an embodiment, the polynucleotide is stably integrated into the genome of the organism or part thereof, preferably into the nuclear genome of the organism or part thereof, for example the non-human eukaryotic organism such as a plant or yeast. Alternatively, the polynucleotide is not integrated into the genome of the organism or part thereof, but is expressed in the organism or part thereof, for example transiently.

In an embodiment, the plant is transgenic for the polynucleotide which is stably integrated into the genome of the plant, wherein the polynucleotide encodes a precursor RNA molecule of the first, second, third, fourth or fifth aspects, or an embodiment of the asymmetric RNA molecules, wherein the precursor RNA molecule and/or at least some of the antisense RNA sequences in the double-stranded product RNA molecules produced from the precursor RNA molecules are capable of reducing the expression and/or activity of a target RNA molecule in the insect, such as for example a Lepidopteran insect, when ingested or in a fungal pathogen. In an embodiment, the transgenic plant has increased resistance to the insect or to the fungal pathogen relative to a corresponding plant lacking the polynucleotide. For example, at least some of the insect larvae are killed after ingesting some of the transgenic plant cells expressing the precursor RNA molecules. In an embodiment, the transgenic plant is a cotton plant and the insect is of the genus Helicoverpa, or a maize or sorghum or rice plant and the insect is of the genus Spodoptera. In an embodiment, the fungal pathogen is of the genus Fusarium or Verticillium.

In an embodiment of the fourth or fifth aspects or the embodiments of the asymmetric precursor RNA molecule, the invention provides a non-human organism, or a part thereof, preferably a plant or part thereof, or an insect or part thereof, or a fungus or part thereof, comprising one or more or all of the precursor RNA molecule, the double-stranded RNA molecule, the population of multiple, different double-stranded RNA molecules, the polynucleotide, the vector, or the host cell of the invention. In an embodiment, the non-human organism or part thereof is a transgenic plant or part thereof, being transgenic for a polynucleotide of the invention, preferably wherein the polynucleotide is stably integrated into the genome of the plant or part thereof.

In an aspect, the present invention provides a method of producing the host cell of the invention, the method comprising introducing into a cell one or more or all of the precursor RNA molecule of the invention, the double-stranded RNA molecule of the invention, the population of multiple, different double-stranded RNA molecules of the invention, the polynucleotide of the invention, or the vector of the invention. In a preferred embodiment, if the host cell is an animal cell, the step of introducing said molecules occurs ex vivo. For example, the introducing step occurs in vitro. The introducing step may be followed by a step of culturing or propagating the molecules into which the molecules were introduced, which may comprise a step of selecting a transformed cell and/or identifying a cell or progeny cell comprising the molecule(s). Progeny cells may be assayed to identify cells having a suitable phenotype.

In an aspect, the present invention provides a method of producing a non-human organism of the invention, the method comprising introducing one or more or all of the precursor RNA molecule of the invention, the double-stranded RNA molecule of the invention, the population of multiple, different double-stranded RNA molecules of the invention, the polynucleotide of the invention, or the vector of the invention, into a cell and generating the non-human organism from the cell. In an embodiment, where the non-human organism is a plant, the step of generating the non-human organism from the cell comprises regenerating a transgenic plant from the cell. The step of introducing a polynucleotide of the invention may be followed by a step of selecting or identifying a cell or progeny cell comprising the polynucleotide.

In an embodiment, the polynucleotide of the invention is stably integrated into the genome of the cell or the organism, preferably into the nuclear genome of the organism or part thereof, preferably a plant.

In an aspect, the present invention provides a method of producing the precursor RNA molecule of the invention, the double-stranded RNA molecule of the invention, and/or the population of multiple, different double-stranded RNA molecules of the invention, the method comprising expressing the polynucleotide of the invention in a host cell or cell-free expression system. In an embodiment, the host cell is a eukaryotic cell, preferably a plant cell or a yeast cell. In an embodiment, the host cell is a cell that has been modified to reduce dsRNA degradation, for example by removal of a Type III ribonuclease.

In an embodiment, the method further comprises extracting and/or at least partially purifying some of the precursor RNA molecule of the invention or double-stranded RNA molecule of the invention or the population of multiple, different double-stranded RNA molecules of the invention. In an embodiment, the method does not comprise a step of extracting the RNA molecules from the host cell. In an embodiment, the host cell is inactivated or killed after the precursor RNA is produced, for example by heat treatment.

In an aspect, the present invention provides an extract of a cell of the invention, wherein the extract comprises one or more or all of the precursor RNA molecule of the invention, the double-stranded RNA molecule of the invention, the population of multiple, different double-stranded RNA molecules of the invention, the polynucleotide of the invention, or the vector of the invention. In an embodiment, the extract comprises the precursor RNA molecule of the invention but not the double-stranded RNA molecules of the invention or the population of multiple, different double-stranded RNA molecules of the invention. In an embodiment, the extract of the cell is purified to remove one or more impurities and/or concentrate the RNA molecules.

In an aspect, the present invention provides a composition comprising one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, or the extract of the invention. In an embodiment, the composition comprises the precursor RNA molecule of the invention but not the double-stranded RNA molecules of the invention or the population of multiple, different double-stranded RNA molecules of the invention. Alternatively, the composition comprises the double-stranded RNA molecules of the invention or the population of multiple, different double-stranded RNA molecules of the invention but not the precursor RNA molecule. In an embodiment, the extract of the cell is purified to remove one or more impurities and/or concentrate the RNA molecules.

In an embodiment, the extract or the composition comprises a population of two, three, four, five, 10, 15, 20, 50, or 100 or more double-stranded RNA molecules of the invention.

In an embodiment, the composition is a pharmaceutical composition.

In an embodiment, the composition is suitable for application to plants growing in a field, such as by spraying or dusting onto plants. For example, the composition comprises a surfactant.

In an embodiment, the composition further comprises at least one compound which enhances the stability, or entry into a eukaryotic cell or both, of the precursor RNA molecule, the double-stranded RNA molecule, population of double-stranded RNA molecules, the polynucleotide, the vector, the host cell, the non-human organism or part thereof, or the extract. An example of such a compound is a transfection promoting agent such as, for example, a detergent.

In an aspect, the present invention provides a method for increasing the number of double-stranded RNA molecules of the invention in a eukaryotic cell or organism, comprising expressing in the cell or organism a polynucleotide of the invention or a vector of the invention, or contacting the cell or organism with the double-stranded RNA molecule of the invention.

In an aspect, the invention provides a method for increasing the total number of sRNA molecules produced in a eukaryotic cell or organism, preferably 22-mers, 23-mers and/or 24-mers, preferably in a plant, fungus, nematode, or an arthropod such as an insect, arachnid, or decapod, the method comprising delivering to the cell or organism one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, the extract of the invention, or the composition of the invention. In an embodiment, the total number sRNA molecules in the cell or organism is assayed. Alternatively, the total number sRNA molecules in the cell or organism is not assayed directly, but a suitable phenotype is observed. Preferably, the number of antisense sRNAs that hybridise to a target RNA molecule of interest is increased. In a related aspect, the invention provides a method for modifying the ratio of sRNA molecules of the invention in a eukaryotic cell or organism, relative to the total number of 21-mer sRNAs, preferably increasing the ratio of 22-mers, 23-mers and/or 24-mers relative to the total number of 21-mer sRNAs, more preferably with regard to a specific target RNA molecule, especially in a plant, fungus or nematode, or an arthropod such as an insect, arachnid, or decapod.

In an embodiment for the precursor RNA molecules of the fourth or fifth aspects or the embodiments of the asymmetric precursor RNA molecule, the invention provides a method for increasing the number of double-stranded RNA molecules of the invention in an insect or an insect cell, or a fungal cell or fungus, comprising expressing in the insect or an insect cell, or fungal cell or fungus a polynucleotide or a vector of the invention, or contacting the insect or an insect cell, or fungal cell or fungus with the double-stranded RNA molecule or the population of double-stranded RNA molecules of the invention.

In an aspect, the invention provides a method for identifying a phenotype or function associated with a target RNA molecule in a eukaryotic cell or organism, the method comprising (i) delivering to the cell or organism, one or more or all of: the precursor RNA molecule of the invention, the double-stranded RNA molecule of the invention, the population of multiple, different double-stranded RNA molecules of the invention, the polynucleotide of the invention, the vector of the invention, the extract of the invention, or the composition of the invention, and (ii) observing the cell or organism, or a progeny cell or organism thereof, for the phenotype or function, or assaying the cell or organism, or a progeny cell or organism thereof, for a molecule associated with the phenotype or function, thereby identifying the phenotype or function associated with a target RNA.

In an embodiment for the precursor RNA molecules of the fourth or fifth aspects or the embodiments of the asymmetric precursor RNA molecule, the invention provides a method for identifying a function or phenotype associated with a target RNA molecule in an insect or insect cell, or a fungal cell or fungus, the method comprising (i) delivering to the insect or insect cell, or fungal cell or fungus, one or more or all of: the precursor RNA molecule, the double-stranded RNA molecule, the population of multiple, different double-stranded RNA molecules, the polynucleotide, the vector, the cell, the non-human organism or part thereof, the extract, and the composition of the invention, and (ii) determining a function or phenotype of the insect or insect cell, or fungal cell or organism, or a progeny insect or fungal cell or progeny insect or progeny fungus thereof, or assaying the insect or insect cell, or fungal cell or fungus, or a progeny insect or insect cell, or a progeny fungal cell or fungus thereof, for a molecule associated with the function or phenotype, thereby identifying the function or phenotype associated with a target RNA.

In an aspect, the invention provides a method for identifying a region of a target RNA molecule in a eukaryotic cell or organism that is susceptible to down-regulation by RNAi, the method comprising (i) delivering to the cell or organism one or more or all of: multiple precursor RNA molecules of the invention, multiple double-stranded RNA molecules of the invention, populations of multiple, different double-stranded RNA molecules of the invention, polynucleotides of the invention, vectors of the invention, extracts of the invention, or compositions of the invention, wherein the multiple precursor RNA molecules, double-stranded RNA molecules or populations of multiple double-stranded RNA molecules target different regions of the target RNA molecule, and (ii) assaying the cell or organism, or a progeny cell or organism thereof, for one or more of: the amount of target RNA molecule, the amount of protein encoded by the target RNA molecule, and/or for a phenotype or function associated with the target RNA molecule, and (iii) selecting a region of the target RNA molecule based on assay results from step (ii), thereby identifying the region.

In an embodiment for the precursor RNA molecules of the fourth or fifth aspects or the embodiments of the asymmetric precursor RNA molecule, the invention provides a method for identifying a region of a target RNA molecule in an insect cell or insect, or a fungal cell or fungus that is susceptible to down-regulation by RNAi, the method comprising (i) delivering to the cell or organism one or more or all of: multiple precursor RNA molecules, multiple double-stranded RNA molecules, populations of multiple, different double-stranded RNA molecules, polynucleotides, vectors, extracts, or compositions of the invention, wherein the multiple precursor RNA molecules, double-stranded RNA molecules or populations of multiple double-stranded RNA molecules target different regions of the target RNA molecule, and (ii) assaying the insect cell or insect, or fungal cell or fungus, or a progeny insect cell or insect, or a progeny fungal cell or fungus thereof, for one or more of: the amount of target RNA molecule, the amount of protein encoded by the target RNA molecule, and/or for a function or phenotype associated with the target RNA molecule, and (iii) selecting a region of the target RNA molecule based on assay results from step (ii), thereby identifying the region.

In an aspect, the invention provides a method for identifying a RNA molecule that is capable of having an effect on a pest or pathogen of a eukaryotic cell or organism, the method comprising (i) delivering to the eukaryotic cell or organism, one or more or all of the precursor RNA molecule of the invention, the double-stranded RNA molecule of the invention, the population of different double-stranded RNA molecules of the invention, the polynucleotide of the invention, the vector of the invention, the extract of the invention, or the composition of the invention, (ii) contacting the cell or organism of step (i), or a progeny cell or organism thereof, with the pest or pathogen, (iii) determining whether or not the precursor RNA molecule, double-stranded RNA molecule or population of different double-stranded RNA molecules has an effect on the pest or pathogen, and optionally (iv) if the precursor RNA molecule, double-stranded RNA molecule or population of different double-stranded RNA molecules has a desirable effect on the pest or pathogen, selecting an RNA molecule based on results from step (iii), thereby identifying the RNA molecule.

In an embodiment for the precursor RNA molecules of the fourth or fifth aspects or the embodiments of the asymmetric precursor RNA molecule, the invention provides a method for identifying an RNA molecule that is capable of having an effect on an insect pest, or a fungal pathogen, the method comprising (i) delivering to the insect pest or fungal pathogen, one or more or all of the precursor RNA molecule, the double-stranded RNA molecule, the population of multiple, different double-stranded RNA molecules, the polynucleotide, the vector, the cell, the non-human organism or part thereof, the extract, and the composition of the invention, and (ii) determining whether or not the precursor RNA molecule, double-stranded RNA molecule or population of different double-stranded RNA molecules has an effect on the insect pest or fungal pathogen, and optionally (iii) if the precursor RNA molecule, double-stranded RNA molecule or population of different double-stranded RNA molecules has a desirable effect on the insect pest or fungal pathogen, selecting an RNA molecule based on results from step (ii), thereby identifying the RNA molecule.

In an aspect, the invention provides a method for identifying a RNA molecule that is capable of having an effect on a pest or pathogen of a eukaryotic cell or organism, the method comprising (i) delivering to the pest or pathogen, one or more or all of the precursor RNA molecule of the invention, the double-stranded RNA molecule of the invention, the population of multiple, different double-stranded RNA molecules of the invention, the extract of the invention, or the composition of the invention, (ii) testing the pest or pathogen for an effect of the precursor RNA molecule, double-stranded RNA molecule or population of double-stranded RNA molecules, and optionally (iii) selecting a RNA molecule based on results from step (ii), thereby identifying the RNA molecule.

In an aspect, the present invention provides a method for reducing or down-regulating the level and/or activity of a target RNA molecule in a eukaryotic cell or organism, the method comprising delivering to the cell or organism one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, the extract of the invention, or the composition of the invention. In an embodiment, the cell or organism is not an animal cell or organism, preferably the cell or organism is a plant or fungal cell or organism. In an embodiment, the cell is a nematode cell such as a plant-pathogenic nematode (PPN) cell, or a nematode, or PPN.

In an embodiment, the method reduces or down-regulates at least two, at least three, at least four, at least five, or at least six different target RNA molecules in a eukaryotic cell using either a single precursor RNA molecule, which may have chimeric target sequences in its dsRNA regions, or a combination of precursor RNA molecules and/or the population of double-stranded RNA molecules of the invention. The different target RNA molecules may be produced by differential splicing from a single gene, or be produced from a gene family. Alternatively, the different target RNA molecules may be unrelated in sequence.

In an embodiment, one or more or all of the precursor RNA molecule, the double-stranded RNA molecule, the population of double-stranded RNA molecules, the polynucleotide, the vector, the extract, or the composition, are contacted with the cell or organism, preferably a plant cell, plant, nematode cell, nematode, fungus, insect cell or insect, by topical application to the cell or organism such as by spraying, dusting or injection, or provided in a feed for the organism. In an embodiment, the composition comprises at least two, at least three, at least four, at least five, or at least six different precursor RNA molecules, each targeting a different target RNA molecule. The different target RNA molecules may be unrelated in sequence.

In an embodiment for the precursor RNA molecules of the fourth or fifth aspects or the embodiments of the asymmetric precursor RNA molecule, the invention provides a method for reducing or down-regulating the level and/or activity of a target RNA molecule in an insect or an insect cell, or a fungal cell or fungus, the method comprising delivering to the insect or an insect cell, or fungal cell or fungus one or more or all of the precursor RNA molecule, the double-stranded RNA molecule, the population of multiple, different double-stranded RNA, the polynucleotide, the vector, the cell, the non-human organism or part thereof, the extract, and the composition of the invention. In an embodiment, one or more or all of the precursor RNA molecule, the double-stranded RNA molecule, the population of double-stranded RNA molecules, the polynucleotide, the vector or the extract are contacted with the insect or insect cell, or fungal cell or fungus by topical application to the insect or an insect cell, or fungal cell or fungus such as by spraying, dusting, soaking, providing in a feed, or applying a composition comprising the precursor RNA molecule, the double-stranded RNA molecule, the population of double-stranded RNA molecules, the polynucleotide, the vector or the extract.

In an aspect, the present invention provides a method for reducing or down-regulating the level and/or activity of a target RNA molecule in an eukaryotic organism, the method comprising orally or parenterally delivering to the organism one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, the extract of the invention, or the composition of the invention. In an embodiment, at least two, at least three, at least four, at least five, or at least six different precursor RNA molecules, each targeting a different target RNA molecule, are delivered. The different target RNA molecules may be unrelated in sequence.

In an aspect, the present invention provides a method of reducing or preventing damage caused by a pest or pathogen to a non-human organism, or to a eukaryotic cell in vitro, the method comprising delivering to the pest or pathogen or cell, or contacting the pest or pathogen or cell with, one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, the extract of the invention, or the composition of the invention. In an embodiment, the RNA molecule targets a target RNA molecule which is involved in feeding, growth, development, perception, movement, reproduction, hormone function, or survival of the pest or pathogen.

In an aspect, the present invention provides a method of controlling a non-human eukaryotic organism, the method comprising delivering to the non-human organism one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, the extract of the invention, or the composition of the invention, wherein the precursor RNA molecule, double-stranded RNA molecule or population of double-stranded RNA molecules has a deleterious effect on the non-human organism. In an embodiment, the organism is a plant and the double-stranded RNA molecule or population of double-stranded RNA molecules reduces the amount and/or activity of a target RNA molecule in the plant, where the target RNA molecule normally functions for the growth, development or reproduction of the plant. In an embodiment, the target RNA molecule functions for photosynthesis or amino acid metabolism, such as for example, EPSP synthase.

In an embodiment, the non-human organism is an arthropod, preferably an insect, a nematode, or a plant.

In an embodiment, the non-human organism is a plant, and the insect or nematode eats the plant or a portion thereof such as a vegetative plant part or feeds on a plant part e.g. roots.

In an embodiment, the method does not comprise delivering the defined substance to a mammal. In an embodiment, the method does not comprise delivering the defined substance to a human.

In an embodiment for the precursor RNA molecules of the fourth or fifth aspects or the embodiments of the asymmetric precursor RNA molecule, the invention provides a method of controlling an insect or a fungus, the method comprising delivering to the insect or fungus one or more or all of the precursor RNA molecule, the double-stranded RNA molecule, the population of multiple, different double-stranded RNA molecules, the polynucleotide, the vector, the cell, the non-human organism or part thereof, the extract, and the composition of the invention, wherein the precursor RNA molecule, double-stranded RNA molecule or population of double-stranded RNA molecules has a deleterious effect on the insect or fungus. In an embodiment, the precursor RNA molecule is produced in a plant, and (i) the insect eats the plant or a portion thereof, or feeds on the plant, or (ii) the fungus infects the plant.

In an aspect, the present invention provides a method of treating a disease in an organism, the method comprising administering to the subject one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, the extract of the invention, or the composition of the invention. In an embodiment, the organism is not an animal, preferably the organism is a plant or fungus.

In an embodiment, one or more or all of the precursor RNA molecule, the double-stranded RNA molecule, the population of double-stranded RNA molecules, the polynucleotide, the vector, the cell, the non-human organism or part thereof, the extract, or the composition, are administered topically, orally or parenterally, such as injected.

In an embodiment, the organism is a vertebrate.

In an embodiment, the organism is a mammal.

In an embodiment, the organism is a human.

In an embodiment, the organism is a plant.

In an embodiment for the precursor RNA molecules of the fourth or fifth aspects or the embodiments of the asymmetric precursor RNA molecule, the invention provides a method of treating a disease in an organism, the method comprising administering to an insect pest or a fungal pathogen of the organism one or more or all of the precursor RNA molecule, the double-stranded RNA molecule, the population of multiple, different double-stranded RNA molecules, the polynucleotide, the vector, the cell, the non-human organism or part thereof, the extract, and the composition of the invention. In an embodiment, the organism is a plant.

In an embodiment, the precursor RNA molecule(s) are produced in a eukaryotic cell or organism, or in a eukaryotic host cell, or by in vitro transcription, wherein the eukaryotic cell is preferably a plant cell, microbial cell such as a fungal cell, preferably a yeast cell. In an embodiment, the sense RNA sequence of the double-stranded RNA region of the precursor RNA molecule, or of the product RNA molecules, is shorter than the antisense RNA sequence, preferably wherein the sense RNA sequence is shorter than the antisense RNA sequence entirely because of the presence of non-basepaired ribonucleotides in the antisense RNA sequence that bulge from the double-stranded RNA region or the product RNA molecules, more preferably wherein the sense RNA sequence has a length which is between 87% and 97%, or 87% and 96%, or 91% and 97%, or 91% and 96%, or more preferably between 94% and 97% or 94% and 96% of the length of the antisense RNA sequence, or the length of the sense RNA sequence is about 21/22, 21/23 or 21/24 of the length of the antisense RNA sequence, calculated as a fraction.

In an aspect, the present invention provides a one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, the extract of the invention, or the composition of the invention, for use in treating a disease in a subject, the double-stranded RNA molecule has a beneficial effect on at least one symptom of the disease.

In an embodiment of the fourth or fifth aspects or the embodiments of the asymmetric precursor RNA molecule, the invention provides one or more or all of the precursor RNA molecules, the double-stranded RNA molecule, the population of multiple, different double-stranded RNA molecules, the polynucleotide, the vector, the cell, the non-human organism or part thereof, the extract, and the composition of the invention, for use in treating a disease caused by a fungal pathogen, wherein the double-stranded RNA molecule or the population of double-stranded RNA molecules has a beneficial effect on at least one symptom of the disease or controls the fungal pathogen.

In an aspect, the present invention provides the use of one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, the extract of the invention, or the composition of the invention, in the manufacture of a medicament for treating a disease, for example a disease caused by a fungal pathogen.

In an aspect, the present invention provides a kit comprising one or more or all of the precursor RNA molecule of the invention, the double-stranded RNA molecule of the invention, the population of double-stranded RNA molecules of the invention, the polynucleotide of the invention, the vector of the invention, the cell of the invention, the non-human organism or part thereof of the invention, the extract of the invention, or the composition of the invention.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1: Synthesis of ledRNA constructs. Schematic designs of two ledRNA molecules: (A) This ledRNA molecule comprises a central sense sequence which can be considered to be two adjacent sense sequences, covalently linked without an intervening spacer sequence and having identity to the target RNA, an antisense sequence which is complementary to the sense sequence and which is divided into two regions, a 5′ region and a 3′ region, which flank the central sense sequence, and two loops that separate the sense from the antisense sequences. (B) This ledRNA molecule comprises a central antisense sequence which can be considered to be two adjacent antisense sequences, covalently linked without an intervening spacer sequence and having identity to the complement of a target RNA, a sense sequence which is complementary to the antisense sequence and which is divided into two regions, a 5′ sense region and a 3′ sense region, which flank the central antisense sequence, and two loops that separate the sense and antisense sequences. The RNA molecule produced by transcription, for example by in vitro transcription from a promoter such as a T7 or SP6 promoter, self-anneals by basepairing between the complementary sense and antisense sequences to form a double-stranded region with a loop at each end and having a “nick” in either the antisense or sense sequence. Additional sequences may be linked to the 5′ and/or 3′ ends as 5′- or 3′-extensions.

FIG. 2: Alignment for the sense sequence of the target region of GUS mRNA (GUS WT; SEQ ID NO: 2) and the modified sense sequence (GUS GU; SEQ ID NO: 3), showing the positions of the C to T substitutions. Asterisks indicate nucleotides that were not changed.

FIG. 3: GUS activity in plants transformed with constructs encoding modified hairpin RNAs for reducing expression of a GUS target gene. The control PPGH11 and PPGH24 plants without the hairpin constructs gave about 4000 MUG units.

FIG. 4: Autoradiogram of a Northern blot of RNAs from six transgenic tobacco plants transformed with a 35S-hpGUS[G:U] construct and six tobacco plants transformed with a corresponding 35S-hpGUS[Con] construct. A. Upper panel shows larger RNAs hybridising to a 200-nucleotide GUS sense probe. Lower panel shows the staining of RNA in the gel prior to blotting, as a loading control. B. Upper panel shows small antisense RNAs hybridising to the sense GUS probe in the same plants as in A. The sample was lost for lane 4 of the hpGUS[G:U] plants resulting in no hybridising signal for that lane. Lower panel shows the same blot probed with an antisense probe to mir168, as a loading control. The lane labelled M was loaded with size markers for 24 nucleotides (nt) and 21 nt.

FIG. 5: Upper panel: Autoradiogram of Northern blot of small RNAs (21-24 nucleotides) from independently transformed plants expressing a 35S-hpGUS[G:U] construct (lanes 1-10) or a 35S-hpGUS[Con] construct (lanes 11-19). The blot was first probed with a sense GUS probe and then re-probed with U6 RNA probe as a loading control. Lower panels: Frequency distribution of sRNAs of 18-25 nucleotides length, calculated from RNA seq analysis (reads per million sequences). The upper part of each panel shows the frequency of sense sRNAs and the lower part antisense sRNAs. The panels labelled hpGUS[G:U]-12 and -13 show data from hpGUS[G:U] plant numbers 12 and 13 from the Northern blot, and the panel labelled hpGUS[Con]-3 shows data from hpGUS[Con] plant number 3.

FIG. 6: Schematic representation of stem-loop structures of transcripts expressed from GUS hpRNA constructs. The transcripts have complementary sense and antisense sequences which basepair to form GUS sequence-specific dsRNA stems, with the indicated lengths in basepairs (bp) for the stems, and the number of nucleotides (nt) in the loops.

FIG. 7: Northern blot analysis showing RNA from expression of the two short hpRNA transgenes GUShp93-1 and GUShp93-2 in stably transformed A. thaliana plants. RNA samples were either treated (+) or not treated (−) with RNase I. The RNA blot was hybridised with a loop-specific antisense RNA probe.

FIG. 8: (A) Schematic map of the genetic construct to express hpPBAN[Con] and hpPBAN[G:U] RNA molecules in plant cells. p35S: 35S promoter of CaMV; pT7: T7 RNA polymerase promoter; OCS-T: transcription terminator/polyadenylation region of the Agrobacterium ocs gene. (B) Alignment of the sense DNA sequences for hpPBAN[Con] (top row, PBAN-WT) and hpPBAN[G:U]. Asterisks show identity of nucleotides, absence of asterisks show the C to T substitutions.

FIG. 9: Northern blot hybridisation to detect hpPBAN RNA transcripts in transgenic N. tabacum plants using an antisense loop sequence (upper panel) or a dsRNA stem sequence (middle panel) as probe. The lower panel shows the stained RNA on the gel before transfer to the Northern blot, as a control for loading. The bands corresponding to the stem-loop molecules (SL) and the processed loop (Loop) are arrowed.

FIG. 10: Northern blot hybridisation to detect sRNA molecules from the dsRNA region of hpPBAN transcripts in transgenic N. tabacum plants to express hpPBAN[Con] or hpPBAN[G:U]. The size markers in the first lane were 21-mer and 24-mer in length.

FIG. 11: Upper panel: Northern blot hybridisation to detect hpPBAN[Con] or hpPBAN[G:U] RNA molecules in independent transgenic A. thaliana plants (lanes hpPBAN[Con] and hpPBAN[G:U]), one N. benthamiana hpPBAN[Con] plant and one hpPBAN[G:U] plant (lanes Nb), one N. tabacum hpPBAN[Con] plant and one hpPBAN[G:U] plant (lanes W38), and S. cerevisiae strain HF7C (yeast) transformed with the construct to express hpPBAN[Con] in one lane or hpPBAN[G:U] RNA in the next lane, using an antisense loop sequence as probe. The A. thaliana plants were T2 plants homozygous for the transgene whereas the N. benthamiana and N. tabacum plants were TO plants. The two lanes at the extreme right contained in vitro produced RNA transcripts to show the position of the band for the full-length transcripts—the position of the main band (arrow) was evident at a shorter exposure time. Lower panel: the gel stained for RNA before blotting to the membrane, to show the RNA loading. The left-most lane in the lower panel shows the size markers (RNA ladder).

FIG. 12: Northern blot hybridisation detection of hpPBAN[Con] and hpPBAN[G:U] derived RNAs in total leaf RNA (Total RNA), loaded at 10 μg per lane, or from nuclear RNA loaded at 1.5 μg per lane (Nuclear RNA). Lanes labelled 7, 12 and 13 were from three N. benthamiana plants independently transformed with the hpPBAN[Con] construct, and lanes labelled 6, 10 and 12 were from three N. benthamiana plants independently transformed with the hpPBAN[G:U] construct. Lanes marked WT were loaded with RNA from a control, non-transgenic plant. The lower panel shows the gel stained for RNA as a loading control, with prominent bands for rRNA.

FIG. 13: Growth test of cotton bollworm larvae on detached leaf pieces from transgenic tobacco plants transformed with the hpPBAN[Con] or hpPBAN[G:U] constructs, compared to leaf pieces from untransformed tobacco plants. The lower panel shows the sizes of the resultant larvae.

FIG. 14: Alignment of the sense DNA sequences for hpAChE[Con] (top row; SEQ ID NO: 17) and hpAChE[G:U] (SEQ ID NO: 18). Vertical dashes show identity of nucleotides, gaps show the C to T substitutions.

FIG. 15: Alignment of the sense DNA sequences for hpHa1[Con] (top row; SEQ ID NO: 23) and hpHa1[G:U] (SEQ ID NO: 24). Vertical dashes show identity of nucleotides, gaps show the 73 C to T substitutions.

FIG. 16: Alignment of the sense DNA sequences for hpHa2[Con] (top row; SEQ ID NO: 29) and hpHa2[G:U] (SEQ ID NO: 30). Vertical dashes show identity of nucleotides, gaps show the 73 C to T substitutions.

FIG. 17: Alignment of the sense DNA sequences for hpHa3[Con] (top row; SEQ ID NO: 35) and hpHa3[G:U] (SEQ ID NO: 36). Vertical dashes show identity of nucleotides, gaps show the 75 C to T substitutions.

FIG. 18: Alignment of the sense DNA sequences for hpHa4[Con] (top row; SEQ ID NO: 41) and hpHa4[G:U] (SEQ ID NO: 42). Vertical dashes show identity of nucleotides, gaps show the 77 C to T substitutions.

FIG. 19: Alignment of the sense DNA sequences for hpHa5[Con] (top row; SEQ ID NO: 47) and hpHa5[G:U] (SEQ ID NO: 48). Vertical dashes show identity of nucleotides, gaps show the 75 C to T substitutions.

FIG. 20: Alignment of the sense DNA sequences for hpHa6[Con] (top row; SEQ ID NO: 53) and hpHa6[G:U] (SEQ ID NO: 54). Vertical dashes show identity of nucleotides, gaps show the 77 C to T substitutions.

FIG. 21: Alignment of the sense DNA sequences for hpHa7[Con] (top row; SEQ ID NO: 59) and hpHa7[G:U] (SEQ ID NO: 60). Vertical dashes show identity of nucleotides, gaps show the 77 C to T substitutions.

FIG. 22: Alignment of the sense DNA sequences for hpHa8[Con] (top row; SEQ ID NO: 65) and hpHa8 [G:U] (SEQ ID NO: 66). Vertical dashes show identity of nucleotides, gaps show the 77 C to T substitutions.

FIG. 23: Relative expression levels of candidate target genes in H. argimera larvae in neonates (Od) and at 1, 3, 5 and 8 days post hatching. Expression levels were normalised to expression of the EF1 gene.

FIG. 24: Upper panel: Photograph of an agarose gel after electrophoresis and staining for RNA of samples of extracted RNA from artificial diet material supplemented with hairpin RNAs transcribed from the hpAChE[Con] (hpCon) or hpAChE[G:U] (hpGU) genetic constructs, or without the RNA (Control), in the absence (−) or in the presence (+) of H. armigera larvae. Lower panel: Autoradiograph of the Northern blot of the same extracted RNA, probed for the hpAChE RNA.

FIG. 25: Left hand panel. Autoradiograph of a Northern blot of extracted RNA from leaves painted with hairpin RNAs transcribed from the hpPBAN[Con] (hpCon) or hpPBAN[G:U] (hpGU) genetic constructs, or without the RNA (Control). The bands for in vitro transcribed RNA show the position of the full-length transcripts. Right hand panel. Autoradiograph of the Northern blot of RNA extracted from larvae fed for 24 hr on the painted leaves, probed for the hpPBAN RNA.

FIG. 26: Quantitation by RT-PCR of target gene transcript levels in H. armigera larvae after feeding them on pakchoi leaves painted with in vitro transcribed hpRNA[G:U] RNA at the stated amount per 0.5 ml per leaf. Larvae were fed for three days (upper and middle panels) or six days (lower panels). Expression levels were normalised to expression of the EF1 gene, as before.

FIG. 27: Mortality rates for H. armigera larvae fed on N. benthamiana leaves expressing hairpin RNAs for 7 days.

FIG. 28: Representative northern blot hybridisations of RNA from TO plants transformed with a genetic construct for expression of hpHa1[G:U] or hpHa1[Con] (upper panel), hpHa6[G:U] or hpHa6[Con] (middle and lower panels). The positions of the presumed full-length hairpin RNA and the loop fragment are arrowed. The lane labelled W38 had RNA from an untransformed plant.

FIG. 29: Average leaf damage scores at day 7 for leaf pieces from transgenic plants transformed with constructs expressing hpHa1[G:U] or hpHa6[G:U] RNA molecules compared to the corresponding, canonically basepaired hpHa1[Con] RNA molecules or the control hpGFP[G:U] molecules. Each bar represents the average score for an independently transformed TO plant.

FIG. 30: Northern blot hybridization shows clear downregulation of Ha6 mRNA in Helicoverpa armigera that have fed for 6 days on transgenic hpHa6[G:U] tobacco leaves. This is in contrast to insects fed on the hpHa6[Con] lines (labelled hpHa1[WT]), showing significantly less Ha6 downregulation. Each RNA sample was extracted from approximately 5 insects that fed on leaves of the same TO plant line.

FIG. 31: Schematic for construction by GoldenGate methods of a binary vector having sequences for a Gemini Virus (GV) construct for expression of RNAi molecules in plant cells. The DNA fragments are mixed, treated with restriction enzyme BsaI and T4 DNA ligase to insert the four components in the order shown into the backbone vector pICH47742rc, replacing the lacZ sequence.

FIG. 32: Northern blot analysis of production in plant cells of a modified hairpin RNA molecule using a Gemini Virus (GV) expression construct compared to a non-replicative vector (NV) expressing the same RNA molecule, after transient expression in N. benthamiana leaves. The right-hand lane shows the RNA produced in a stably transformed tobacco plant. The CMV 2b silencing suppressor protein was either present (+) or absent (−) as indicated. The lower panel shows the stained RNA in the agarose gel prior to blotting, as loading control.

FIG. 33: Schematic of asymmetric hairpin RNA (hpRNA) molecules transcribed from genetic constructs comprising sense sequences containing regularly spaced single, double or triple nucleotide deletions, targeting a transgene encoding GUS for silencing. The construct encoding hpGUS[Con] had the wild-type sense sequence from the GUS target region, whereas the construct encoding hpGUS[G:U] had the same sense sequence except that each C nucleotide was substituted with a T nucleotide, producing G:U non-canonical basepairs in the hpGUS[G:U]; these two constructs were used as controls. The siRNAs produced from each hpGUS molecule are shown schematically toward the left, indicating hybridised sense (upper) and antisense (lower) strands. The free energy of folding of each of the transcripts (kcal/mol) are shown at the right. Downward arrowheads indicate the positions of the unpaired nucleotides that bulge out from the dsRNA regions, and the X symbols in the siRNAs indicate positions of deletions in the modified sense sequences of the siRNAs.

FIG. 34: Alignment of the nucleotide sequence (sense sequence) of a region of the GUS gene (upper sequence GUS[Con]; SEQ ID NO: 2) targeted by a hpRNA and the modified sense sequences (lower sequence) of the hpGUS[Δ22] construct (GUS[Δ22]; SEQ ID NO: 95), the hpGUS[Δ23] construct (GUS[Δ23]; SEQ ID NO: 96), the hpGUS[Δ24-1] construct (GUS[Δ24-1]; SEQ ID NO: 97), the hpGUS[Δ24-2] construct (GUS[Δ24-2]; SEQ ID NO: 98) or the hpGUS[Δ24-3] construct (GUS[Δ24-3]; SEQ ID NO: 99). Retained nucleotides are asterisked, nucleotides deleted in the modified sense sequences of the hpGUS construct are indicated with a dash.

FIG. 35: Expression analysis. (A) Northern blot of small antisense RNAs expressed from the hpGUS transgenes agroinfiltrated into N. benthamiana leaves, probed with the sense sequence from the hpGUS RNA molecules. The lower panel of A shows the same Northern blot hybridised with a probe that was complementary to mir168 as a loading control. (B) Northern blot of small antisense RNAs produced in hpGUS transgenic tobacco lines. The lower panel of B shows the same Northern blot hybridised with a probe that was complementary to U6 rRNA as a loading control. (C) Northern blot of small sense RNAs produced in hpGUS transgenic tobacco lines. The lower panel of C shows the same Northern blot hybridised with a probe that was complementary to mir168 as a loading control. (D) GUS activity of 14 different TO transgenic plants for each genetic construct, measured by MUG assay of leaf samples.

FIG. 36: Deep sequencing analysis of doubly transgenic tobacco plants transformed with a GUS target gene and a hpGUS construct, as indicated above each frame. (A) Reads (reads per million) for sRNAs of 19-25 nucleotides length mapped to the 200-nucleotide target region of the GUS gene. (B) Reads (reads per million) for sRNAs of 19-25 nucleotides length downstream of the target region, The rightward three frames in the upper panel are expanded in the middle panel. The bars show the frequencies of sense or antisense sRNAs which are 19, 20, 21, 22, 23, 24 or 25 nucleotides in length, per million reads in the total sRNA populations. Error bars=s.e.m.

FIG. 37: Deep sequencing reads for the total number of sRNAs of 19-25 nucleotides length mapping to the GUS target transcript, where the sRNAs were produced in tobacco plants transformed with a GUS target gene and a hpGUS construct as indicated. The plots show the positions in the GUS transcript where the sRNAs mapped to and the frequency at each position. The dotted lines show the position of the 200-nucleotide target region.

FIG. 38: Alignment of the nucleotide sequence (sense sequence) of a region of the EIN2 gene (upper sequence EIN2[Con]; SEQ ID NO: 110) targeted by a hpRNA and the modified sense sequences (lower sequence) of the hpEIN2[Δ22] construct (EIN2[Δ22]; SEQ ID NO: 111), the hpEIN2[Δ23] construct (EIN2[Δ23]; SEQ ID NO: 112), the hpEIN2[Δ24-1] construct (EIN2[Δ24-1]; SEQ ID NO: 113), the hpEIN2[Δ24-2] construct (EIN2[Δ24-2]; SEQ ID NO: 114) or the hpEIN2[Δ24-3] construct (EIN2[Δ24-3]; SEQ ID NO: 115). Retained nucleotides are asterisked, nucleotides deleted in the modified sense sequences of the hpEIN2 construct are indicated with a dash.

FIG. 39: Analysis of transformed plants (A) Average hypocotyl lengths for A. thaliana seedlings transformed with asymmetric hpEIN2 constructs, compared to the untransformed seedlings (wildtype) or the conventional hpEIN2[Con] construct as a control. (B) Normalised EIN2 transcript levels in the same batches of transformed plants. Error bars=s.e.m.

FIG. 40: Deep sequencing reads for transgenic A. thaliana plants transformed with a hpEIN2 construct, as indicated above each frame. (A) Reads (reads per million) for sRNAs of 19-25 nucleotides length mapped to the 200-nucleotide target region of the EIN2 gene. (B) Reads (reads per million) for sRNAs of 19-25 nucleotides length downstream of the target region. The frames labelled WT were for the wild-type (untransformed) control plants, and hpEIN2[Con] were for plants transformed with the conventional hairpin construct. The bars show the frequencies of sense or antisense sRNAs which are 19, 20, 21, 22, 23, 24 or 25 nucleotides in length, per million reads in the total sRNA populations. Error bars=s.e.m.

FIG. 41: Deep sequencing reads of sRNAs were produced in A. thaliana plants transformed with a hpEIN2 construct as indicated. Deep sequencing reads for the total number of sRNAs of 19-25 nucleotides length mapping to the EIN2 target transcript. The plots show the positions in the EIN2 transcript where the sRNAs mapped to and the frequency at each position. The dotted lines show the position of the 200-nucleotide target region.

FIG. 42: Alignment of the nucleotide sequence of a chimeric sense sequence formed by joining 300 nucleotides of CMV 2b gene with 300 nucleotides of the cDNA from PVY (upper sequence; SEQ ID NO: 117) and the modified sense sequence (lower sequence; SEQ ID NO: 119) of the hpCMV/PVY[Δ22] construct. Retained nucleotides are asterisked, nucleotides deleted in hpCMV/PVY[Δ22] are indicated with a dash.

FIG. 43: Northern blot analyses. Panel A: Northern blot of RNA from plants infected with CMV, probed for viral RNAs, showing the positions of CMV RNA3 and RNA4 (CP). Panel B. Photo of the stained gel for Panel A prior to blotting, showing the loading of the lanes and the rRNA bands. Panel C. Northern blot of sRNAs hybridizing to the target region of the CMV, showing the positions of 21 nt, 22 nt and 24 nt bands. Panel D. The same blot as in Panel C but hybridized with a probe to detect rRNA U6, for loading control. The lanes are labelled with the construct in the transgenic plants from which the RNAs were prepared; WT=wild-type, non-transgenic plants as control. The left-hand most WT lanes were from wild-type control plants that were not infected with CMV.

FIG. 44. Alignment of the chimeric sense sequence formed by joining to regions of the B. napus DDM1 gene (upper sequence DDM1[Con]; SEQ ID NO: 122) included in the control construct hpDDM1[Con] and in ledDDM1[Con], and the modified sense sequence (lower sequence) of the ledDDM1[Δ22] construct (SEQ ID NO: 125). Identical nucleotides are shown by vertical strokes; nucleotides deleted in the modified sense sequence of the ledDDM1[Δ22] construct are indicated with a dash.

FIG. 45. qRT-PCR measurement of DDM1 mRNA transcript abundance in B. napus floral tissue after treatment with ledDDM1[Con] (ledDDM1) or ledDDM1[Δ22] (ledDDM1[22 nt]) RNAs.

FIG. 46: Demonstration of root uptake and systemic movement of ledDDM1[Con] molecules after topical application. The canola roots were in contact with the RNA solution for six hours before being analysed for uptake of ledDDM1[Con] and reduction of DDM1 mRNA in root and cotyledons.

FIG. 47: Systemic silencing and movement of ledRNA molecules taken up through the roots. (A) 1% agarose gel demonstrating integrity and amount of the ledGUS (control), ledDDM1[Con] and ledDDM1[Δ22] molecules. (B) Initial experiment demonstrating enhanced silencing of ledDDM1[Δ22] compared to ledDDM1[Con]. (C) Northern blot demonstrating uptake of the RNAi molecules into the roots and movement into the cotyledons. D) qRT-PCR demonstrating reduction of DDM1 mRNA in roots and cotyledons.

FIG. 48: Demonstration of enhanced silencing and systemic movement of RNA molecules from cotyledon into adjacent tissues. (C) Northern blot of RNA from untreated cotyledons and roots showing presence of RNA molecules in those organs. (D) Reduction of the target mRNA at 6 hr, 24 hr and 6 days as measured by qRT-PCR.

FIG. 49: Alignment of the modified sense sequence (SEQ ID NO: 139), used in NbSu Construct 2 having the Δ22 modification (NbSu-Δ22), with the wild-type sense sequence (NbSu-WT; SEQ ID NO: 129). The positions of the single nucleotide deletions shown as dashes.

FIG. 50: Alignment of the modified sense sequence (SEQ ID NO: 140), used in NbSu Constructs 3 and 4 having the Δ22 and A to G substitution modifications (NbSu-Δ22AG), with the wild-type sense sequence (NbSu-WT; SEQ ID NO: 129). The positions of the single nucleotide deletions shown as dashes and the substitutions in grey.

FIG. 51: Alignment of the modified antisense sequence (SEQ ID NO: 141), used in NbSu Construct 4 having the C to T substitutions (NbSu-Δ22CT) with the unmodified antisense sequence (NbSu-AS; SEQ ID NO: 130) used in Constructs 1, 2 and 3. The positions of the 24 nucleotide substitutions shown in grey.

FIG. 52: Schematic design of genetic constructs to express a hairpin RNA molecule (Panel A) or ledRNA molecule (Panel B) either in vitro using a T7 RNA Polymerase promoter or in vivo using a CaMV 35S promoter. The linear arrangements show the DNA elements of the genetic constructs, the lower schematic structures show the hpRNA or ledRNA molecules after transcription of the genetic constructs, splicing of the Cat-1 introns when transcribed in a eukaryotic cell, and folding of the RNA strand by hybridisation between complementary sense and antisense strands to produce the hpRNA or ledRNA molecules. The 5′ and 3′ ends of the RNA molecules are indicated. There is a single-strand nick between 3′ and 5′ ends of the ledRNA (not shown). The GGG trinucleotide at the 5′ end was added to provide for efficient in vitro transcription using T7 RNA Polymerase, with the complementary CCC trinucleotide inserted at the 3′ end of the hairpin molecule or into the antisense strand of the ledRNA construct.

FIG. 53: Schematic representations of structures of asymmetric RNA molecules. Showing a precursor RNA molecule (A) in the upper part of each panel A-Z and the resultant double-stranded product RNA molecules (P) after cleavage by a Dicer in the lower part. The extent of the double-stranded region (B) comprising the first RNA strand (D) and second RNA strand (F) is shown, joined by a linking RNA sequence (Loop (L)). In each panel A-Z, the double-stranded region (B) can be extended leftward and/or rightward by addition of ribonucleotides, as desired. The position of the loop at the right-hand end of each precursor molecule implies a 5′ to 3′ order of the sequences as (D)-loop-(F), whereas if the loop (L) is present at the left-hand end of the double-stranded region to join the strands, 5′ to 3′ order would be (F)-loop-(D); this is a possible variation for all panels A-Z. The arbitrary position and length of the first RNA sequence (E) within the first RNA strand (D) and the second RNA sequence (G) within the second RNA strand (F) are shown—the position and length of (E) and (G) within (D) and (F), respectively, can be changed arbitrarily, for example can be considered to occupy the whole of (D) and (F), respectively. Ribonucleotides are shown as N,N′ or B. N and N′ with downward strokes represent basepairs when the first RNA strand (D) and second RNA strand (F) hybridise, independently forming either canonical basepairs or non-canonical basepairs such as G:U basepairs. Ribonucleotides shown as B bulge out from the double-stranded structure. Each horizontal dash represents the absence of a ribonucleotide opposite a B ribonucleotide, engineered through either the deletion of a ribonucleotide at the dash position or the insertion of the ribonucleotide shown as B. Each B opposite another B represents a mismatched (non-basepaired) ribonucleotide pair. Vertical arrows represent exemplary positions for cleavage by a Dicer, at either closed arrows or open arrows. The positions of cleavage can be moved leftward or rightward within the double-stranded region (B), with a first cleavage anywhere between the loop (L) and the first bulged nucleotide, provided at least some of product molecules (P) are produced having the same lengths of sense RNA sequence (H) and antisense RNA sequence (J) i.e. 21/22-mer, 21/23-mer or 21/24-mers. When Dicer cleaves at the exemplary positions shown with closed arrows, the product RNA molecules (P) are shown below the long, closed arrow; analogously the cleavage at the positions shown by open arrows produces product RNA molecules (P) shown below the long, open arrow. Panels A-E and F-H represent exemplary structures that produce at least some 21/22-mer siRNAs (P), panels I-N and O-Q represent exemplary structures that produce at least some 21/23-mer siRNAs (P), and panels R-V and U-Z represent exemplary structures that produce at least some 21/24-mer siRNAs (P). Panels F-H, O-Q and U-Z represent shorter structures where a ribonuclease such as Drosha is capable of cleaving off 5′ leader and 3′ trailer sequences. In panels F-H, O-Q and U-Z, the double-stranded regions can be extended by basepairing between ribonucleotides N2 and N3, and further between ribonucleotides N1 and N4, or extended even further. In each of panels A-Z, the positions of the non-basepaired nucleotide(s) B can be varied as desired. Product RNA molecules (P) have two-ribonucleotide 3′ overhangs that are not basepaired. The 5′ and 3′ ends are shown. Panel A: the first RNA sequence (E) has one non-basepaired ribonucleotide and the second RNA sequence (G) has two non-basepaired ribonucleotides, including a mismatched pair, forming two bulges in the double-stranded region. Panel B: the first RNA sequence (E) has one non-basepaired ribonucleotide and the second RNA sequence (G) has two non-basepaired ribonucleotides, including a mismatched pair, forming one bulge in the double-stranded region. Panel C: the first RNA sequence (E) has one non-basepaired ribonucleotide and the second RNA sequence (G) has two non-basepaired ribonucleotides, forming three single-ribonucleotide bulges in the double-stranded region. Panel D: the first RNA sequence (E) has no non-basepaired ribonucleotides and the second RNA sequence (G) has one non-basepaired ribonucleotide, forming one bulge in the double-stranded region. Panel E: shows an extended double-stranded structure where the modifications are concatemerized. Each first RNA sequence (E) has no non-basepaired ribonucleotides and each second RNA sequence (G) has one non-basepaired ribonucleotide, forming one bulge in the double-stranded region about every 22 ribonucleotides. The bulges are separated by 21 contiguous basepairs, but that number can be varied to provide an average separation of the bulges of about 21 basepairs. Panel F: the first RNA sequence (E) has one non-basepaired ribonucleotide and the second RNA sequence (G) has two non-basepaired ribonucleotides, including a mismatched pair, not counting ribonucleotides N1-N4 which may or may not be basepaired, forming two bulges in the double-stranded region. Panel G: the first RNA sequence (E) has one non-basepaired ribonucleotide and the second RNA sequence (G) has two non-basepaired ribonucleotides, not counting ribonucleotides N1-N4 which may or may not be basepaired, forming three bulges in the double-stranded region. Panel H: the first RNA sequence (E) has no non-basepaired ribonucleotides and the second RNA sequence (G) has one non-basepaired ribonucleotide, not counting ribonucleotides N1-N4 which may or may not be basepaired, forming one bulge in the double-stranded region. Panel I: the first RNA sequence (E) has one non-basepaired ribonucleotide and the second RNA sequence (G) has two non-basepaired ribonucleotides, including a mismatched pair, forming two bulges in the double-stranded region. Produces at least some 21/23-mers. Panel J: the first RNA sequence (E) has one non-basepaired ribonucleotide and the second RNA sequence (G) has three non-basepaired ribonucleotides, forming four bulges in the double-stranded region. Produces at least some 21/23-mers. Panel K: the first RNA sequence (E) has no non-basepaired ribonucleotides and the second RNA sequence (G) has two non-basepaired ribonucleotides, forming a single di-ribonucleotide bulge in the double-stranded region. Produces at least some 21/23-mers. Panel L: the first RNA sequence (E) has no non-basepaired ribonucleotides and the second RNA sequence (G) has two non-basepaired ribonucleotides, forming two single-ribonucleotide bulges in the double-stranded region. Produces at least some 21/23-mers. Panel M: shows an extended double-stranded structure where the modifications are concatemerized. Each first RNA sequence (E) has no non-basepaired ribonucleotides and each second RNA sequence (G) has two non-basepaired ribonucleotides, forming two bulges in the double-stranded region, then concatemerized. The bulges are each separated by 10 or 11 contiguous basepairs, but that number can be varied to provide a separation of two bulges about, on average, every 23 ribonucleotides. Produces at least some 21/23-mers. Panel N: shows an extended double-stranded structure where the modifications are concatemerized. Each first RNA sequence (E) has no non-basepaired ribonucleotides and each second RNA sequence (G) has two non-basepaired ribonucleotides, forming a di-ribonucleotide bulge in double-stranded region, then concatemerized. The bulges are each separated by 21 contiguous basepairs, but that number can be varied to provide a separation between bulges of, on average, about 21 basepairs. Produces at least some 21/23-mers. A hybrid structure between those shown in panels M and N would have single and di-ribonucleotide bulges. Panel O: the first RNA sequence (E) has one non-basepaired ribonucleotide and the second RNA sequence (G) has three non-basepaired ribonucleotides, including a mismatched pair, not counting ribonucleotides N1-N4 which may or may not be basepaired, forming two bulges in the double-stranded region. Produces at least some 21/23-mers. Panel P: the first RNA sequence (E) has no non-basepaired ribonucleotides and the second RNA sequence (G) has two non-basepaired ribonucleotides, not counting ribonucleotides N1-N4 which may or may not be basepaired, forming a di-ribonucleotide bulge in the double-stranded region. Produces at least some 21/23-mers. Panel Q: the first RNA sequence (E) has no non-basepaired ribonucleotides and the second RNA sequence (G) has two non-basepaired ribonucleotides, not counting ribonucleotides N1-N4 which may or may not be basepaired, forming two single-ribonucleotide bulges in the double-stranded region. Produces at least some 21/23-mers. Panel R: the first RNA sequence (E) has one non-basepaired ribonucleotide and the second RNA sequence (G) has four non-basepaired ribonucleotides, including a mismatched pair, forming two bulges in the double-stranded region including a tri-ribonucleotide bulge. Produces at least some 21/24-mers. Panel S: the first RNA sequence (E) has one non-basepaired ribonucleotide and the second RNA sequence (G) has four non-basepaired ribonucleotides, forming five bulges in the double-stranded region, all single-ribonucleotide bulges. Produces at least some 21/24-mers. Panel T: extended double-stranded region, showing how multiple structures can be formed for product RNA molecules (P). The first RNA sequence (E) has no non-basepaired ribonucleotides and the second RNA sequence (G) in each case has three non-basepaired ribonucleotides, forming one tri-ribonucleotide bulge and three single-ribonucleotide bulges in the double-stranded region. Produces at least some 21/24-mers. Panel U: extended double-stranded region, showing how multiple structures can be formed for product RNA molecules (P). The first RNA sequence (E) has no non-basepaired ribonucleotides and the second RNA sequence (G) in each case has three non-basepaired ribonucleotides, forming one tri-ribonucleotide bulge, a single-ribonucleotide bulge and a di-ribonucleotide bulge in the double-stranded region. The order of these bulges can be re-arranged, provided the second RNA sequence (G) in each case has, on average, three non-basepaired ribonucleotides about every 24 ribonucleotides. Produces at least some 21/24-mers. Panel V: extended form, concatemerized. The first RNA sequence (E) has no non-basepaired ribonucleotides and the second RNA sequence (G) has three non-basepaired ribonucleotides, forming a tri-ribonucleotide bulge, on average, about every 24 ribonucleotides in the double-stranded region. Produces at least some 21/24-mers. Panel W: the first RNA sequence (E) has one non-basepaired ribonucleotide and the second RNA sequence (G) has four non-basepaired ribonucleotides, including a mismatched pair, not counting ribonucleotides N1-N4 which may or may not be basepaired, forming two bulges in the double-stranded region. Produces at least some 21/24-mers. Panel X: the first RNA sequence (E) has no non-basepaired ribonucleotides and the second RNA sequence (G) has three non-basepaired ribonucleotides, not counting ribonucleotides N1-N4 which may or may not be basepaired, forming a single tri-ribonucleotide bulge in the double-stranded region. Produces at least some 21/24-mers. Panel Y: the first RNA sequence (E) has no non-basepaired ribonucleotides and the second RNA sequence (G) has three non-basepaired ribonucleotide, not counting ribonucleotides N1-N4 which may or may not be basepaired, forming two bulges in the double-stranded region, including one single ribonucleotide bulge and the other a di-ribonucleotide bulge. Produces at least some 21/24-mers. Panel Z: the first RNA sequence (E) has no non-basepaired ribonucleotides and the second RNA sequence (G) has three non-basepaired ribonucleotide, not counting ribonucleotides N1-N4 which may or may not be basepaired, forming three single-ribonucleotide bulges in the double-stranded region. Produces at least some 21/24-mers.

FIG. 54. Variant chimeric RNA molecules having additional target transcript sequences. The loop sequence of hpNbSu[Con] (Example 24) was substituted with a 400 nt unmodified sense sequence to make Construct 27.1 encoding hpNbSu[Con]-Loop1, or a chimeric 500 nt sense sequence to make Construct 27.2 encoding hpNbSu[Con]-Loop2. In analogous fashion, the loop sequence of hpNbSu[G:U] was substituted with the 400 nt unmodified sense sequence to make Construct 27.3 encoding hpNbSu[G:U]-Loop1, and the loop sequence of hpNbSu[Δ22] was substituted with the 400 nt unmodified sense sequence to make Construct 27.4 encoding hpNbSu[Δ22]-Loop1 or with the chimeric 500 nt sense sequence to make Construct 27.5 encoding hpNbSu[Δ22]-Loop2. The hpNbSu[Δ22] molecule comprising the Cat-1 intron as a loop sequence was further modified by adding the chimeric 500 nt sequence to the 3′ end of the antisense sequence as a 3′ extension, generating Construct 27.6 encoding hpNbSu[Δ22]-ext.

FIG. 55. Resistance of plants to fungal infection. The graph shows the mean percentage of transgenic plants with yellowing or necrotic leaves 16 days post inoculation with F. oxysporum. Mean taken of two replicates of 10 plants per pot for each line.

FIG. 56. Representative northern blot hybridisation of RNA from T1 transgenic tobacco (W38) or N. benthamiana (Nb) plants transformed with a genetic construct for expression of hpHa5[Con] or hpHa5[G:U]. The position of the presumed full-length hairpin RNA is arrowed. The lane labelled W38 had RNA from an untransformed tobacco plant.

### KEY TO THE SEQUENCE LISTING

SEQ ID NO: 1. Nucleotide sequence of the protein coding region of the cDNA corresponding to the mRNA encoding GUS; 1812 nt.

SEQ ID NO: 2. Nucleotide sequence of the GUS target sequence, included as the sense sequence for a conventional construct encoding hpGUS[Con]; 200 nt.

SEQ ID NO: 3. Nucleotide sequence of the sense sequence for the construct encoding hpGUS[G:U], containing 52 C to T substitutions relative to SEQ ID NO: 2; 200 nt.

SEQ ID NO: 4. Nucleotide sequence (RNA) of the hairpin structure of the hpGUS[Con] RNA, including its loop sequence after splicing of the intron and the restriction enzyme sites used for cloning purposes. The sense sequence is nucleotides 7-206 and the antisense sequence is nucleotides 258-457; 463 nt.

SEQ ID NO: 5. Nucleotide sequence (RNA) of the hairpin structure of the hpGUS[G:U] RNA, including its loop sequence after splicing of the intron and the restriction enzyme sites used for cloning purposes. The sense sequence is nucleotides 7-206 and the antisense sequence is nucleotides 246-451; 457 nt.

SEQ ID NO: 6. Nucleotide sequence encoding stem-loop structure of hairpin RNAs hpGUS93-1 and hpGUS93-2. Nucleotides 1-11 and 285-295, restriction enzyme sites; nucleotides 8-100 are the 93-nucleotide sense sequence corresponding to nucleotides 512-604 of SEQ ID NO: 1, nucleotides 101-195 are a spacer sequence corresponding to nucleotides 610-700 of SEQ ID NO: 1, and nucleotides 196-288 are the 93-nucleotide antisense sequence complementary to nucleotides 512-604 of SEQ ID NO: 1; 295 nt.

SEQ ID NO: 7. Amino acid sequence of Helicoverpa armigera Pheromone Biosynthesis Activating Neuropeptide (PBAN) polypeptide; NCBI Reference Sequence: XP_021199198; 174aa.

SEQ ID NO: 8. Nucleotide sequence of a cDNA for Helicoverpa armigera PBAN, through to translation stop codon; Genbank Accession No. XM_021343523.1 (LOC110382821). The protein coding region corresponds to nucleotides 36-560; 560 nt.

SEQ ID NO: 9. Nucleotide sequence of a 280-nucleotide target region of the RNA for H. armigera PBAN; 280 nt.

SEQ ID NO: 10. Nucleotide sequence of a modified 280-nucleotide sense region corresponding to the RNA for H. armigera PBAN, having 64 cytosines substituted with thymidines; 280 nt.

SEQ ID NO: 11. Nucleotide sequence of a DNA molecule encoding hpPBAN[Con] for targeting the transcript of the gene encoding H. armigera PBAN. The order of elements are: Restriction sites for BamHI (nucleotides 1-6), XhoI (6-11), T7 promoter (12-31), PBAN antisense sequence (32-311), loop sequence (312-461), PBAN sense sequence (462-741), restriction sites for SmaI (742-747) and HindIII (748-754). The region of the mRNA used as the sense sequence was nucleotides 36-315, the loop sequence was nucleotides 316-465; 754 nt.

SEQ ID NO: 12. Nucleotide sequence encoding hpPBAN[G:U] targeting the transcript of the gene encoding H. armigera PBAN. The order of elements are: restriction sites for BamHI (nucleotides 1-6), XhoI (6-11), T7 promoter (12-31), PBAN antisense sequence (32-311), loop sequence (312-461), PBAN sense sequence (462-741), restriction sites for SmaI (742-747) and HindIII (748-754); 754 nt.

SEQ ID NO: 13. Nucleotide sequence of the protein coding region of a construct p35S-CMV-2b encoding a CMV 2b silencing suppressor polypeptide; 333 nt.

SEQ ID NO: 14. Amino acid sequence of the CMV 2b silencing suppressor polypeptide (Goto et al., 2007); 111aa.

SEQ ID NO: 15. Amino acid sequence of H. armigera acetylcholinesterase AChE1; NCBI Accession No. AAM90333.1 (Ren et al., 2002); 646aa.

SEQ ID NO: 16. Nucleotide sequence of a cDNA for H. armigera acetylcholinesterase mRNA; GenBank Accession No. AF369793.1; Ren et al., (2002). The protein coding region is nucleotides 316-2259; 2551 nt.

SEQ ID NO: 17. Nucleotide sequence of a 494-nucleotide target region of the cDNA for the mRNA encoding H. armigera acetylcholinesterase, corresponding to nucleotides 895-1388 of SEQ ID NO: 16; 494 nt.

SEQ ID NO: 18. Nucleotide sequence of a modified 494 nucleotide sense region corresponding to the RNA for H. armigera acetylcholinesterase, having 86 cytosines substituted with thymidines relative to the wild-type sequence; 494 nt.

SEQ ID NO: 19. Nucleotide sequence of a DNA molecule encoding hpAChE[Con] for targeting the transcript of the AChE1 gene encoding H. armigera acetylcholinesterase. The order of elements are: Restriction sites for BamHI (nucleotides 1-6), XhoI (6-11), T7 promoter (12-31), AChE antisense sequence (32-525), loop sequence (526-787), AChE sense sequence (788-1281), restriction sites for SmaI (1282-1287) and HindIII (1288-1293); 1294 nt.

SEQ ID NO: 20. Nucleotide sequence of a DNA molecule encoding hpAChE[G:U] for targeting the transcript of the AChE1 gene encoding H. armigera acetylcholinesterase. The order of elements are: Restriction sites for BamHI (nucleotides 1-6), XhoI (6-11), T7 promoter (12-31), AChE antisense sequence (32-525), loop sequence (526-787), modified AChE sense sequence (788-1281), restriction sites for SmaI (1282-1287) and HindIII (1288-1293); 1294 nt.

SEQ ID NO: 21. Amino acid sequence of H. armigera ecdysone receptor EcR (Ha1); NCBI Genbank Accession No. ASK12085.1; 584aa.

SEQ ID NO: 22. Nucleotide sequence of a cDNA for H. armigera ecdysone receptor EcR mRNA; GenBank Accession No. KY328717.1. The protein coding region is nucleotides 255-2009; 2407 nt.

SEQ ID NO: 23. Nucleotide sequence of a 300-nucleotide target region of the cDNA for a mRNA encoding H. armigera ecdysone receptor EcR, corresponding to nucleotides 1240-1539 of SEQ ID NO: 22; 300 nt.

SEQ ID NO: 24. Nucleotide sequence of a modified 300-nucleotide sense region corresponding to the RNA for H. armigera ecdysone receptor, having 73 cytosines substituted with thymidines (24.3%) relative to the wild-type sequence; 300 nt.

SEQ ID NO: 25. Nucleotide sequence of a DNA molecule encoding hpHa1[Con] for targeting the transcript of the EcR gene encoding H. armigera ecdysone receptor. The order of elements: Restriction site for XhoI (nucleotides 3-8), T7 promoter (9-28), EcR (Ha1) antisense sequence (29-328), loop sequence (329-478), EcR sense sequence (479-778), restriction sites for SmaI (779-784) and XbaI (787-792); 794 nt.

SEQ ID NO: 26. Nucleotide sequence of a DNA molecule encoding hpHa1[G:U] for targeting the transcript of the EcR gene encoding H. armigera ecdysone receptor. The order of elements are: Restriction site for XhoI (nucleotides 3-8), T7 promoter (9-28), EcR (Ha1) antisense sequence (29-328), loop sequence (329-478), modified EcR sense sequence (479-778), restriction sites for SmaI (779-784) and XbaI (787-792); 794 nt.

SEQ ID NO: 27. Amino acid sequence of H. armigera coatomer beta subunit (Ha2); NCBI Genbank Accession No. XP_021194683.1 (LOC110379367); encoded by transcript variant X2; 950aa.

SEQ ID NO: 28. Nucleotide sequence of a cDNA for H. armigera coatomer beta subunit mRNA; GenBank Accession No. XM_021339008.1. The protein coding region is nucleotides 88-2940; 3057 nt.

SEQ ID NO: 29. Nucleotide sequence of a 300-nucleotide target region of the cDNA for the mRNA encoding H. armigera coatomer-β, corresponding to nucleotides 1632-1931 of SEQ ID NO: 28; 300 nt.

SEQ ID NO: 30. Nucleotide sequence of a modified 300-nucleotide sense region corresponding to the RNA for H. armigera coatomer-β, having 73 cytosines substituted with thymidines (24.3%) relative to the wild-type sequence; 300 nt.

SEQ ID NO: 31. Nucleotide sequence of a DNA molecule encoding hpHa2[Con] for targeting the transcript of the coatomer beta gene encoding H. armigera coatomer beta subunit. The order of elements are: Restriction site for XhoI (nucleotides 3-8), T7 promoter (9-28), Ha2 antisense sequence (29-328), loop sequence (329-478), Ha2 sense sequence (479-778), restriction sites for SmaI (779-784) and XbaI (787-792); 794 nt.

SEQ ID NO: 32. Nucleotide sequence of a DNA molecule encoding hpHa2[G:U] for targeting the transcript of the coatomer beta gene encoding H. armigera coatomer beta subunit. The order of elements are: Restriction site for XhoI (nucleotides 3-8), T7 promoter (9-28), Ha2 antisense sequence (29-328), loop sequence (329-478), modified Ha2 sense sequence (479-778), restriction sites for SmaI (779-784) and XbaI (787-792); 794 nt.

SEQ ID NO: 33. Amino acid sequence of H. armigera molt-regulating transcription factor (HR3), referred to herein as Ha3; NCBI Genbank Accession No. ACH86113.1; 556aa.

SEQ ID NO: 34. Nucleotide sequence of the protein coding region of a cDNA for H. armigera molt-regulating transcription factor (Ha3) mRNA; GenBank Accession No. FJ009448.1. The protein coding region is nucleotides 1-1671; 1671 nt.

SEQ ID NO: 35. Nucleotide sequence of a 300-nucleotide target region of the cDNA for the mRNA encoding H. armigera Molt-regulating transcription factor (Ha3), corresponding to nucleotides 48-347 of SEQ ID NO: 34; 300 nt.

SEQ ID NO: 36. Nucleotide sequence of a modified 300-nucleotide sense region corresponding to the RNA for H. armigera Molt-regulating transcription factor (Ha3), having 75 cytidines substituted with thymidines (25.0%) relative to the wild-type sequence; 300 nt.

SEQ ID NO: 37. Nucleotide sequence of a DNA molecule encoding hpHa3[Con] for targeting the transcript of the Molt-regulating transcription factor gene encoding H. armigera Molt-regulating transcription factor. The order of elements are: Restriction site for XhoI (nucleotides 3-8), T7 promoter (9-28), Ha3 antisense sequence (29-328), loop sequence (329-478), Ha3 sense sequence (479-778), restriction sites for SmaI (779-784) and XbaI (787-792); 794 nt.

SEQ ID NO: 38. Nucleotide sequence of a DNA molecule encoding hpHa3[G:U] for targeting the transcript of the Molt-regulating transcription factor gene encoding H. armigera Molt-regulating transcription factor. The order of elements are: Restriction site for XhoI (nucleotides 3-8), T7 promoter (9-28), Ha3 antisense sequence (29-328), loop sequence (329-478), modified Ha3 sense sequence (479-778), restriction sites for SmaI (779-784) and XbaI (787-792); 794 nt.

SEQ ID NO: 39. Amino acid sequence of H. armigera V-type proton ATPase catalytic subunit A (Ha4); NCBI Genbank Accession No. XP_021181049.1 (LOC110369820), encoded by transcript variant X1; 620aa.

SEQ ID NO: 40. Nucleotide sequence of a cDNA for H. armigera V-type proton ATPase catalytic subunit A mRNA; GenBank Accession No. XM_021325374.1 (LOC110369820), transcript variant X1. The protein coding region is nucleotides 144-2006; 2645 nt.

SEQ ID NO: 41. Nucleotide sequence of a 300-nucleotide target region of the cDNA for the mRNA encoding H. armigera V-type proton ATPase catalytic subunit A, corresponding to nucleotides 164-463 of SEQ ID NO: 40; 300 nt.

SEQ ID NO: 42. Nucleotide sequence of a modified 300-nucleotide sense region corresponding to the RNA for H. armigera V-type proton ATPase catalytic subunit A, having 77 cytosines substituted with thymidines (25.7%) relative to the wild-type sequence; 300 nt.

SEQ ID NO: 43. Nucleotide sequence of a DNA molecule encoding hpHa4[Con] for targeting the transcript of the V-type proton ATPase catalytic subunit A gene encoding H. armigera V-type proton ATPase catalytic subunit A. The order of elements are: Restriction site for XhoI (nucleotides 3-8), T7 promoter (9-28), Ha4 antisense sequence (29-328), loop sequence (329-478), Ha4 sense sequence (479-778), restriction sites for SmaI (779-784) and XbaI (787-792); 794 nt.

SEQ ID NO: 44. Nucleotide sequence of a DNA molecule encoding hpHa4[G:U] for targeting the transcript of the V-type proton ATPase catalytic subunit A gene encoding H. armigera V-type proton ATPase catalytic subunit A. The order of elements are: Restriction site for XhoI (nucleotides 3-8), T7 promoter (9-28), Ha3 antisense sequence (29-328), loop sequence (329-478), modified Ha3 sense sequence (479-778), restriction sites for SmaI (779-784) and XbaI (787-792); 794 nt.

SEQ ID NO: 45. Amino acid sequence of H. armigera trypsin-like serine protease (Ha5); NCBI Genbank Accession No. ACJ66841.1; 299aa. The signal peptide consists of the first 20 amino acids. The trypsin-like serine protease domain is amino acids 28-274; 299aa.

SEQ ID NO: 46. Nucleotide sequence of a cDNA for H. armigera trypsin-like serine protease mRNA; GenBank Accession No. EU874846.1. The protein coding region is nucleotides 32-931; 1025 nt.

SEQ ID NO: 47. Nucleotide sequence of a 300-nucleotide target region of the cDNA for the mRNA encoding H. armigera trypsin-like serine protease, corresponding to nucleotides 621-920 of SEQ ID NO: 46; 300 nt.

SEQ ID NO: 48. Nucleotide sequence of a modified 300-nucleotide sense region corresponding to the RNA for H. armigera Trypsin-like serine protease, having 75 cytosines substituted with thymidines (25.0%) relative to the wild-type sequence; 300 nt.

SEQ ID NO: 49. Nucleotide sequence of a DNA molecule encoding hpHa5[Con] for targeting the transcript of the Trypsin-like serine protease gene encoding H. armigera Trypsin-like serine protease. The order of elements are: Restriction site for XhoI (nucleotides 3-8), T7 promoter (9-28), Ha5 antisense sequence (29-328), loop sequence (329-478), Ha5 sense sequence (479-778), restriction sites for SmaI (779-784) and XbaI (787-792); 794 nt.

SEQ ID NO: 50. Nucleotide sequence of a DNA molecule encoding hpHa5[G:U] for targeting the transcript of the Trypsin-like serine protease gene encoding H. armigera Trypsin-like serine protease. The order of elements are: Restriction site for XhoI (nucleotides 3-8), T7 promoter (9-28), Ha5 antisense sequence (29-328), loop sequence (329-478), modified Ha5 sense sequence (479-778), restriction sites for SmaI (779-784) and XbaI (787-792); 794 nt.

SEQ ID NO: 51. Amino acid sequence of H. armigera synaptic vesicle glycoprotein 2C-like, encoded by transcript variant X2 of gene LOC110370333 (Ha6); NCBI Genbank Accession No. XP_021181756.1; 557aa.

SEQ ID NO: 52. Nucleotide sequence of a cDNA for H. armigera synaptic vesicle glycoprotein 2C-like mRNA; GenBank Accession No. XM_021326081. The protein coding region is nucleotides 386-2059; 2661 nt.

SEQ ID NO: 53. Nucleotide sequence of a 300-nucleotide target region of the cDNA for the mRNA encoding H. armigera synaptic vesicle glycoprotein 2C-like, corresponding to nucleotides 1010-1309 of SEQ ID NO: 52; 300 nt.

SEQ ID NO: 54. Nucleotide sequence of a modified 300-nucleotide sense region corresponding to the RNA for H. armigera synaptic vesicle glycoprotein 2C-like, having 77 cytosines substituted with thymidines (25.7%) relative to the wild-type sequence; 300 nt.

SEQ ID NO: 55. Nucleotide sequence of a DNA molecule encoding hpHa6[Con] for targeting the transcript of the synaptic vesicle glycoprotein 2C-like gene encoding H. armigera synaptic vesicle glycoprotein 2C-like. The order of elements are: Restriction site for XhoI (nucleotides 3-8), T7 promoter (9-28), Ha6 antisense sequence (29-328), loop sequence (329-478), Ha6 sense sequence (479-778), restriction sites for SmaI (779-784) and XbaI (787-792); 794 nt.

SEQ ID NO: 56. Nucleotide sequence of a DNA molecule encoding hpHa6[G:U] for targeting the transcript of the synaptic vesicle glycoprotein 2C-like gene encoding H. armigera synaptic vesicle glycoprotein 2C-like. The order of elements are: Restriction site for XhoI (nucleotides 3-8), T7 promoter (9-28), Ha6 antisense sequence (29-328), loop sequence (329-478), modified Ha6 sense sequence (479-778), restriction sites for SmaI (779-784) and XbaI (787-792); 794 nt.

SEQ ID NO: 57. Amino acid sequence of H. armigera Troponin C, encoded by transcript variant X2 of gene (LOC110380220) (Ha7); NCBI Genbank Accession No. XP_021195809.1; 151aa.

SEQ ID NO: 58. Nucleotide sequence of a cDNA for H. armigera Troponin C mRNA; GenBank Accession No. XM_021340134.1 (LOC110380220). The protein coding region is nucleotides 132-587; 955 nt.

SEQ ID NO: 59. Nucleotide sequence of a 300-nucleotide target region of the cDNA for the mRNA encoding H. armigera Troponin C, corresponding to nucleotides 140-439 of SEQ ID NO: 58; 300 nt.

SEQ ID NO: 60. Nucleotide sequence of a modified 300-nucleotide sense region corresponding to the RNA for H. armigera Troponin C, having 77 cytosines substituted with thymidines (25.7%) relative to the wild-type sequence; 300 nt.

SEQ ID NO: 61. Nucleotide sequence of a DNA molecule encoding hpHa7[Con] for targeting the transcript of the Troponin C gene encoding H. armigera Troponin C. The order of elements are: Restriction site for XhoI (nucleotides 3-8), T7 promoter (9-28), Ha7 antisense sequence (29-328), loop sequence (329-476), Ha7 sense sequence (477-776), restriction sites for SmaI (777-782) and XbaI (785-790); 792 nt.

SEQ ID NO: 62. Nucleotide sequence of a DNA molecule encoding hpHa7[G:U] for targeting the transcript of the Troponin C gene encoding H. armigera Troponin C. The order of elements are: Restriction site for XhoI (nucleotides 3-8), T7 promoter (9-28), Ha7 antisense sequence (29-328), loop sequence (329-476), modified Ha7 sense sequence (477-776), restriction sites for SmaI (777-782) and XbaI (785-790); 792 nt.

SEQ ID NO: 63. Amino acid sequence of H. armigera Titin, encoded by transcript variant X6 of gene (LOC110380881) (Ha8); NCBI Genbank Accession No. XP_021196691.1; 4213aa.

SEQ ID NO: 64. Nucleotide sequence of a cDNA for H. armigera Titin mRNA; GenBank Accession No. XM_021341016.1 (LOC110380881). The protein coding region is nucleotides 365-13006; 13053 nt.

SEQ ID NO: 65. Nucleotide sequence of a 300-nucleotide target region of the cDNA for the mRNA encoding H. armigera Titin, corresponding to nucleotides 520-819 of SEQ ID NO: 64; 300 nt.

SEQ ID NO: 66. Nucleotide sequence of a modified 300-nucleotide sense region corresponding to the RNA for H. armigera Titin, having 77 cytosines substituted with thymidines (25.7%) relative to the wild-type sequence; 300 nt.

SEQ ID NO: 67. Nucleotide sequence of a DNA molecule encoding hpHa8[Con] for targeting the transcript of the Titin gene encoding H. armigera Titin. The order of elements are: Restriction site for XhoI (nucleotides 3-8), T7 promoter (9-28), Ha8 antisense sequence (29-328), loop sequence (329-476), Ha8 sense sequence (477-776), restriction sites for SmaI (777-782) and XbaI (785-790); 793 nt.

SEQ ID NO: 68. Nucleotide sequence of a DNA molecule encoding hpHa8 [G:U] for targeting the transcript of the Titin gene encoding H. armigera Titin. The order of elements are: Restriction site for XhoI (nucleotides 3-8), T7 promoter (9-28), Ha8 antisense sequence (29-328), loop sequence (329-476), modified Ha8 sense sequence (477-776), restriction sites for SmaI (777-782) and XbaI (785-790); 793 nt.

SEQ ID NOs: 69-90. Oligonucleotide primers.

SEQ ID NO: 91. Nucleotide sequence of the DNA fragment for the LIR module comprising the LIR from BeYDV; BsaI restriction sites are present at nucleotides 9-14 and 319-324, spanning the LIR region, including the invariant 9 nucleotides of the LIR at positions 179-187; 332 nt.

SEQ ID NO: 92. Nucleotide sequence of the DNA fragment for the Pr/UTR module comprising the CaMV e35S promoter and TMV 5′UTR regions, Fragment number EN38509. BsaI restriction sites are present at nucleotides 9-14 and 864-869, the e35S promoter at nucleotides 13-767 and the TMV 5′UTR at positions 796-852; the translation start ATG for the S module polypeptide is at nucleotides 860-862; 877 nt.

SEQ ID NO: 93. Nucleotide sequence of the DNA fragment for the T module comprising the CaMV 35S Tm transcription terminator and SIR/Rep/RepA/LIR regions from Gemini Virus BeYDV, used in GV vectors herein, number EN38511. BsaI restriction sites are present at nucleotides 9-14 and 1766-1771, the 35S Tm at nucleotides 20-223, the SIR at positions 224-375, the Rep/RepA coding region in reverse orientation from nucleotides 1466 to 376, and an LIR at positions 1467-1760. The thymidine at nucleotide position 1469 was replaced with a guanosine. Nucleotide position 1469 is a G in GVc and an A for GVt; 1779 nt.

SEQ ID NO: 94. Nucleotide sequence of the DNA fragment encoding a hpHa1[G:U] molecule, used in construction of a GV vector for expressing the RNA molecule in plant cells. BsaI restriction sites are present at nucleotides 9-14 and 812-817, the Ha1 antisense sequence at nucleotides 35-334, the Ha1 sense sequence having 73 cytosines substituted with thymidines (24.3%) relative to the wild-type sequence at nucleotides 485-784, and the sequence encoding the loop at nucleotides 335-484; 825 nt.

SEQ ID NO: 95. DNA sequence of a modified sense sequence from the GUS gene as used in the hpGUS[Δ22] construct; 191 nt.

SEQ ID NO: 96. DNA sequence of a modified sense sequence from the GUS gene as used in the hpGUS[Δ23] construct; 183 nt.

SEQ ID NO: 97. DNA sequence of a modified sense sequence from the GUS gene as used in the hpGUS[Δ24-1] construct; 177 nt.

SEQ ID NO: 98. DNA sequence of a modified sense sequence from the GUS gene as used in the hpGUS[Δ24-2] construct; 176 nt.

SEQ ID NO: 99. DNA sequence of a modified sense sequence from the GUS gene as used in the hpGUS[Δ24-3] construct; 176 nt.

SEQ ID NO: 100. DNA sequence encoding the hpGUS[Con] hairpin RNA molecule. Nucleotides 1-11, 212-223, 1018-1029 and 1230-1235 correspond to restriction enzyme sites from the cloning vector, nucleotides 12-211 correspond to the GUS sense sequence, nucleotides 232-998 correspond to a PDK intron, and nucleotides 1030-1229 correspond to the GUS antisense sequence. The other nucleotides flanking the intron were from the cloning vector; 1235 nt.

SEQ ID NO: 101. DNA sequence corresponding to the hpGUS[Con] hairpin RNA molecule after removal of the PDK intron by splicing, excluding additional 5′ and 3′ extensions coming from the expression vector. Nucleotides 1-11, 212-223, 251-262 and 463-468 correspond to restriction enzyme sites from the cloning vector, nucleotides 12-211 correspond to the GUS sense sequence, and nucleotides 263-462 correspond to the GUS antisense sequence. Nucleotides 224-250 were from the cloning vector and form part of the loop of hpGUS[Con]; 468 nt.

SEQ ID NO: 102. DNA sequence corresponding to the hpGUS[G:U] hairpin RNA molecule after removal of the PDK intron by splicing, excluding additional 5′ and 3′ extensions coming from the expression vector. Nucleotides 1-6, 207-212, 240-251 and 452-457 correspond to restriction enzyme sites from the cloning vector, nucleotides 7-206 correspond to the modified GUS sense sequence, and nucleotides 252-451 correspond to the GUS antisense sequence. Nucleotides 213-239 were from the cloning vector and form part of the loop of hpGUS[G:U]; 457 nt.

SEQ ID NO: 103. DNA sequence corresponding to the hpGUS[Δ22] hairpin RNA molecule after removal of the PDK intron by splicing, excluding additional 5′ and 3′ extensions coming from the expression vector. Nucleotides 1-6, 198-203, 231-242 and 443-448 correspond to restriction enzyme sites from the cloning vector, nucleotides 7-197 correspond to the modified GUS sense sequence, and nucleotides 243-442 correspond to the GUS antisense sequence. Nucleotides 204-230 were from the cloning vector and form part of the loop of hpGUS[Δ22]; 448 nt.

SEQ ID NO: 104. DNA sequence corresponding to the hpGUS[Δ23] hairpin RNA molecule after removal of the PDK intron by splicing, excluding additional 5′ and 3′ extensions coming from the expression vector. Nucleotides 1-6, 190-195, 223-234 and 435-440 correspond to restriction enzyme sites from the cloning vector, nucleotides 7-189 correspond to the modified GUS sense sequence, and nucleotides 235-434 correspond to the GUS antisense sequence. Nucleotides 196-222 were from the cloning vector and form part of the loop of hpGUS[Δ23]; 440 nt.

SEQ ID NO: 105. DNA sequence corresponding to the hpGUS[Δ24-1] hairpin RNA molecule after removal of the PDK intron by splicing, excluding additional 5′ and 3′ extensions coming from the expression vector. Nucleotides 1-6, 184-189, 217-228 and 429-434 correspond to restriction enzyme sites from the cloning vector, nucleotides 7-183 correspond to the modified GUS sense sequence, and nucleotides 229-428 correspond to the GUS antisense sequence. Nucleotides 190-216 were from the cloning vector and form part of the loop of hpGUS[Δ24-1]; 434 nt.

SEQ ID NO: 106. DNA sequence corresponding to the hpGUS[Δ24-2] hairpin RNA molecule after removal of the PDK intron by splicing, excluding additional 5′ and 3′ extensions coming from the expression vector. Nucleotides 1-6, 183-188, 216-227 and 428-433 correspond to restriction enzyme sites from the cloning vector, nucleotides 7-182 correspond to the modified GUS sense sequence, and nucleotides 228-427 correspond to the GUS antisense sequence. Nucleotides 189-215 were from the cloning vector and form part of the loop of hpGUS[Δ24-2]; 433 nt.

SEQ ID NO: 107. DNA sequence corresponding to the hpGUS[Δ24-3] hairpin RNA molecule after removal of the PDK intron by splicing, excluding additional 5′ and 3′ extensions coming from the expression vector. Nucleotides 1-6, 183-188, 216-227 and 428-433 correspond to restriction enzyme sites from the cloning vector, nucleotides 7-182 correspond to the modified GUS sense sequence, and nucleotides 228-427 correspond to the GUS antisense sequence. Nucleotides 189-215 were from the cloning vector and form part of the loop of hpGUS[Δ24-3]; 433 nt.

SEQ ID NO: 108. Nucleotide sequence of the cDNA corresponding to the A. thaliana EIN2 gene, Accession No. NM_120406. Nucleotides 629-4513 correspond to the protein coding region; 4851 nt.

SEQ ID NO: 109. Nucleotide sequence of the cDNA corresponding to A. thaliana CHS gene, Accession No. NM_121396, Nucleotides 287-1471 correspond to the protein coding region; 1703 nt.

SEQ ID NO: 110. DNA sequence of a 200-nucleotide target region of the A. thaliana EIN2 gene, in sense orientation, used as a target sequence; corresponding to nucleotides 654-853 of SEQ ID NO: 108; 200 nt.

SEQ ID NO: 111. Nucleotide sequence of the sense fragment in the construct encoding hpEIN2[Δ22], generated by deleting every 22nd nucleotide from the EIN2 wild-type sense sequence used in the conventional hairpin construct; 191 nt.

SEQ ID NO: 112. Nucleotide sequence encoding the sense sequence in the construct encoding hpEIN2[Δ23], generated by deleting every 11th nucleotide from the EIN2 wild-type sense sequence used in the conventional hairpin construct; 183 nt.

SEQ ID NO: 113. Nucleotide sequence encoding the sense sequence in the construct encoding hpEIN2[Δ24-1], generated by deleting every 7th or 8th nucleotide from the EIN2 wild-type sense sequence used in the conventional hairpin construct; 177 nt.

SEQ ID NO: 114. Nucleotide sequence encoding the sense sequence in the construct encoding hpEIN2[Δ24-2], generated by deleting three nucleotides per 24 nucleotides from the EIN2 wild-type sense sequence used in the conventional hairpin construct, by alternating 1 nt and 2 nt deletions; 176 nt.

SEQ ID NO: 115. Nucleotide sequence encoding the sense sequence in the construct encoding hpEIN2[Δ24-3], generated by deleting every 22nd, 23rd and 24th nucleotides from each 24-nucleotide window in the EIN2 wild-type sense sequence used in the conventional hairpin construct; 176 nt.

SEQ ID NO: 116. Nucleotide sequence of a DNA fragment comprising a 200-nt sense sequence from the cDNA corresponding to A. thaliana CHS gene, used as a target sequence. The sense sequence corresponds to nucleotides 863-1062 of the cDNA sequence (SEQ ID NO: 109); 200 nt.

SEQ ID NO: 117. Nucleotide sequence of a DNA fragment consisting of a 300-nt sense sequence from CMV 2b gene joined to a 300-nt sense sequence from the cDNA for PVY, used as a target sequence. The PVY sense sequence corresponds to nucleotides 6217-6516 of Accession No. NC_001616; 600 nt.

SEQ ID NO: 118. Nucleotide sequence of a DNA fragment encoding hpCMV/PVY[Con]. Nucleotides 1-18 and 1482-1499 corresponding to restriction sites from the cloning vector, nucleotides 26-42 correspond to a T7 RNA Polymerase promoter for in vitro transcription, nucleotides 46-345 correspond to a CMV 2b sense sequence, nucleotides 346-645 correspond to a PVY sense sequence, nucleotides 652-841 correspond to a CAT-1 intron, nucleotides 842-1141 correspond to a PVY antisense sequence, and nucleotides 1142-1441 correspond to a CMV 2b antisense sequence; 1493 nt.

SEQ ID NO: 119. Nucleotide sequence of a DNA fragment comprising a modified sense sequence from a CMV 2b gene with single nucleotide deletions spaced on average about every 22 nucleotides, joined to a modified sense sequence from the cDNA for PVY with single nucleotide deletions spaced on average about every 22 nucleotides, used as the sense sequence in the genetic construct encoding hpCMV/PVY[Δ22]; 574 nt.

SEQ ID NO: 120. Nucleotide sequence of a DNA fragment encoding hpCMV/PVY[Δ22]. Nucleotides 1-12 and 1406-1417 corresponding to restriction sites from the cloning vector, nucleotides 13-29 correspond to a T7 RNA Polymerase promoter for in vitro transcription, nucleotides 30-319 correspond to a modified CMV 2b sense sequence, nucleotides 320-606 correspond to a modified PVY sense sequence, nucleotides 613-802 correspond to a CAT-1 intron, nucleotides 803-1102 correspond to a PVY antisense sequence, and nucleotides 1103-1399 correspond to a CMV 2b antisense sequence; 1416 nt.

SEQ ID NO: 121. Nucleotide sequence of the protein coding region of the cDNA corresponding to the BnaA07g37430D-1 DDM1 gene (LOC106391353) on the A07 chromosome of B. napus. The protein coding region is nucleotides 145-2469; 2653 nt.

SEQ ID NO: 122. Nucleotide sequence of a chimeric sense sequence corresponding to two regions of the B. napus DDM1 gene transcript, joined together. This sequence corresponds to nucleotides 648-959 and nucleotides 2029-2218 of SEQ ID NO: 121; 502 nt.

SEQ ID NO: 123. Nucleotide sequence of DNA encoding hpDDM1[Con] targeting RNA transcripts of the four DDM1 genes of B. napus. Restriction enzyme sites, nucleotides 1-14 and 1807-1824; T7 RNA polymerase promoter with GGG tri-nucleotide, nucleotides 15-34; DDM1 chimeric sense sequence, nucleotides 35-536; loop sequence from pHellsgate 8 cloning vector comprising the PDK1 intron, nucleotides 537-1304; DDM1 chimeric antisense sequence, nucleotides 1305-1806; 1824 nt.

SEQ ID NO: 124. Nucleotide sequence of DNA encoding ledDDM1[Con] targeting RNA transcripts of the four DDM1 genes of B. napus. Restriction enzyme sites, nucleotides 1-14 and 1296-1312; T7 RNA polymerase promoter with GGG tri-nucleotide, nucleotides 16-35; DDM1 antisense sequence, nucleotides 36-281; loop sequence, nucleotides 282-413; DDM1 chimeric sense sequence, nucleotides 414-912; loop sequence, nucleotides 913-1042; DDM1 antisense sequence, nucleotides 1043-1292; 1314 nt.

SEQ ID NO: 125. Nucleotide sequence of a chimeric sense sequence corresponding to two regions of the B. napus DDM1 gene transcript, joined together, having the Δ22 modifications; 480 nt.

SEQ ID NO: 126. Nucleotide sequence of DNA encoding ledDDM1[Δ22] targeting RNA transcripts of the four DDM1 genes of B. napus. Restriction enzyme sites, nucleotides 1-14 and 1296-1312; T7 RNA polymerase promoter with GGG tri-nucleotide, nucleotides 16-35; DDM1 antisense sequence, nucleotides 36-281; loop sequence, nucleotides 282-411; DDM1 chimeric sense sequence with Δ22 modification, nucleotides 412-891; loop sequence, nucleotides 892-1020; DDM1 antisense sequence, nucleotides 1021-1270; 1292 nt.

SEQ ID NO: 127. Nucleotide sequence of a cDNA encoding magnesium-chelatase subunit CHL1 in N. benthamiana (Nbv5.1tr6204879). The protein coding sequence corresponds to nucleotides 141-1424; 1691 nt.

SEQ ID NO: 128. Amino acid sequence of a magnesium-chelatase subunit CHL1 in N. benthamiana (Nbv5.1tr6204879); 427aa.

SEQ ID NO: 129. Nucleotide sequence of a selected sense sequence used as a target sequence for the NbSu mRNA. This sequence corresponds to nucleotides 463-854 of SEQ ID NO: 127; 392 nt.

SEQ ID NO: 130. Nucleotide sequence of an antisense sequence complementary to SEQ ID NO: 129; 392 nt.

SEQ ID NO: 131. Nucleotide sequence of Construct 24.1 encoding hpNbSu[Con] RNA, a symmetric hairpin RNA targeting the Su transcript in N. benthamiana. The order and position of the elements are: T7 promoter (nucleotides 1-17)—sense sequence (nucleotides 21-412)—linker comprising a Cat-1 intron (nucleotides 413-608)—antisense sequence (nucleotides 609-1000)—SmaI restriction enzyme site (nucleotides 1001-1006); 1006 nt.

SEQ ID NO: 132. Nucleotide sequence of Construct 24.2 encoding hpNbSu[Δ22] RNA, an asymmetric hairpin RNA with the Δ22 modification, targeting the Su transcript in N. benthamiana. The order and position of the elements are: T7 promoter (nucleotides 1-17)—sense sequence (nucleotides 21-394)—linker comprising a Cat-1 intron (nucleotides 395-590)—antisense sequence (nucleotides 591-982)—SmaI restriction enzyme site (nucleotides 983-988); 988 nt.

SEQ ID NO: 133. Nucleotide sequence of Construct 24.3 encoding hpNbSu[Δ22AG] RNA, an asymmetric hairpin RNA with the Δ22 modification and 38 A to G substitutions in the sense sequence, targeting the Su transcript in N. benthamiana. The order and position of the elements are: T7 promoter (nucleotides 1-17)—sense sequence (nucleotides 21-394)—linker comprising a Cat-1 intron (nucleotides 395-590)—antisense sequence (nucleotides 591-982)—SmaI restriction enzyme site (nucleotides 983-988); 988 nt.

SEQ ID NO: 134. Nucleotide sequence of Construct 24.4 encoding hpNbSu[Δ22CT] RNA, an asymmetric hairpin RNA with the Δ22 modification, 38 A to G substitutions in the sense sequence and 24 C to T substitutions in the antisense sequence, targeting the Su transcript in N. benthamiana. The order and position of the elements are: T7 promoter (nucleotides 1-17)—sense sequence (nucleotides 21-394)—linker comprising a Cat-1 intron (nucleotides 395-590)—antisense sequence (nucleotides 591-982)—SmaI restriction enzyme site (nucleotides 983-988); 988 nt.

SEQ ID NO: 135. Nucleotide (DNA) sequence corresponding to the hpNbSu[Con] molecule produced from Construct 24.1, after splicing out of the intron, not showing the 5′ leader and 3′ trailer sequences resulting from in planta expression. The linking sequence is nucleotides 396-401; 796 nt.

SEQ ID NO: 136. Nucleotide (DNA) sequence corresponding to the hpNbSu[Δ22] molecule produced from Construct 24.2, after splicing out of the intron, not showing the 5′ leader and 3′ trailer sequences resulting from in planta expression. The linking sequence is nucleotides 378-383; 778 nt

SEQ ID NO: 137. Nucleotide (DNA) sequence corresponding to the hpNbSu[Δ22AG] molecule produced from Construct 24.3, after splicing out of the intron, not showing the 5′ leader and 3′ trailer sequences resulting from in planta expression. The linking sequence is nucleotides 378-383; 778 nt.

SEQ ID NO: 138. Nucleotide (DNA) sequence corresponding to the hpNbSu[Δ22CT] molecule produced from Construct 24.4, after splicing out of the intron, not showing the 5′ leader and 3′ trailer sequences resulting from in planta expression. The linking sequence is nucleotides 378-383; 778 nt.

SEQ ID NO: 139. Nucleotide sequence of the sense sequence having the Δ22 modifications, used in NbSu Construct 24.2; 374 nt.

SEQ ID NO: 140. Nucleotide sequence of the sense sequence having the Δ22 and A to G modifications, used in NbSu Constructs 24.3 and 24.4; 374 nt.

SEQ ID NO: 141. Nucleotide sequence of the antisense sequence having the C to T modifications, used in NbSu Construct 24.4; 392 nt.

SEQ ID NO: 142. Nucleotide sequence of the sense-1 sequence used in NbSu Construct 24.5. This sequence corresponds to nucleotides 463-662 of SEQ ID NO: 127. 200 nt.

SEQ ID NO: 143. Nucleotide sequence of the sense-2 sequence used in Construct 24.5. This sequence corresponds to nucleotides 663-854 of SEQ ID NO: 127; 192 nt.

SEQ ID NO: 144. Nucleotide sequence in 5′ to 3′ orientation of the antisense-2 sequence used in NbSu Constructs 24.5, 24.6 and 24.7, complementary to nucleotides 854-663 of SEQ ID NO: 127; 192 nt.

SEQ ID NO: 145. Nucleotide sequence in 5′ to 3′ orientation of the antisense-1 sequence used in NbSu Constructs 24.5, 24.6 and 24.7, complementary to nucleotides 662-463 of SEQ ID NO: 127; 200 nt.

SEQ ID NO: 146. Nucleotide sequence of the sense-1 sequence used in NbSu Construct 24.6 for ledNbSu[Δ22]; 191 nt.

SEQ ID NO: 147. Nucleotide sequence of the sense-2 sequence used in NbSu Construct 24.6 for ledNbSu[Δ22]; 183 nt.

SEQ ID NO: 148. Nucleotide sequence of the sense-1 sequence used in NbSu Constructs 24.7 and 24.8 for ledNbSu[Δ22AG] and ledNbSu[Δ22CT], respectively; 191 nt.

SEQ ID NO: 149. Nucleotide sequence of the sense-2 sequence used in NbSu Constructs 24.7 and 24.8 for ledNbSu[Δ22AG] and ledNbSu[Δ22CT], respectively; 183 nt.

SEQ ID NO: 150. Nucleotide sequence in 5′ to 3′ orientation of the antisense-2 sequence used in NbSu Construct 24.8; 192 nt.

SEQ ID NO: 151. Nucleotide sequence in 5′ to 3′ orientation of the antisense-1 sequence used in NbSu Construct 24.8; 200 nt.

SEQ ID NO: 152. Nucleotide sequence of NbSu Construct 24.5 encoding ledNbSu[Con] RNA, a symmetric ledRNA targeting the Su transcript in N. benthamiana. The order and position of the elements are: T7 promoter (nucleotides 1-17) sense-2 sequence (nucleotides 21-212)—linker comprising a Cat-1 intron (nucleotides 213-408)—antisense-2 sequence (nucleotides 409-600)—antisense-1 sequence (nucleotides 604-803)—linker comprising a Cat-1 intron (nucleotides 804-999)—sense-1 sequence (nucleotides 1000-1199)—StuI restriction enzyme site (nucleotides 1200-1205). The two antisense sequences are separated by a CCC trinucleotide to be complementary to the GGG trinucleotide after the T7 RNA promoter to increase in vitro transcription efficiency; 1205 nt

SEQ ID NO: 153. Nucleotide sequence of NbSu Construct 24.6 encoding ledNbSu[Δ22] RNA, an asymmetric ledRNA with the Δ22 modification, targeting the Su transcript in N. benthamiana. The order and position of the elements are: T7 promoter (nucleotides 1-17)—sense-2 sequence (nucleotides 21-203)—linker comprising a Cat-1 intron (nucleotides 204-399)—antisense-2 sequence (nucleotides 400-591)—antisense-1 sequence (nucleotides 595-794)—linker comprising a Cat-1 intron (nucleotides 795-990)—sense-1 sequence (nucleotides 991-1181)—StuI restriction enzyme site (nucleotides 1182-1187). Eighteen single-nucleotide deletions in total were made in the sense sequences, every 22^{nd} nucleotide; 1187 nt.

SEQ ID NO: 154. Nucleotide sequence of NbSu Construct 24.7 encoding ledNbSu[Δ22AG] RNA, an asymmetric ledRNA with the Δ22 modification and 38 A to G substitutions in the sense sequence, targeting the Su transcript in N. benthamiana. The order and position of the elements are: T7 promoter (nucleotides 1-17)—sense-2 sequence (nucleotides 21-203)—linker comprising a Cat-1 intron (nucleotides 204-399)—antisense-2 sequence (nucleotides 400-591)—antisense-1 sequence (nucleotides 595-794)—linker comprising a Cat-1 intron (nucleotides 795-990)—sense-1 sequence (nucleotides 991-1181)—StuI restriction enzyme site (nucleotides 1182-1187). Thirty-eight A nucleotides in total in the sense sequences were substituted with G nucleotides; 1187 nt.

SEQ ID NO: 155. Nucleotide sequence of NbSu Construct 24.8 encoding ledNbSu[Δ22CT] RNA, an asymmetric ledRNA with the Δ22 modification, 38 A to G substitutions in the sense sequence and 24 C to T substitutions in the antisense sequence, targeting the Su transcript in N. benthamiana. The order and position of the elements are: T7 promoter (nucleotides 1-17)—sense-2 sequence (nucleotides 21-203)—linker comprising a Cat-1 intron (nucleotides 204-399)—antisense-2 sequence (nucleotides 400-591)—antisense-1 sequence (nucleotides 595-794)—linker comprising a Cat-1 intron (nucleotides 795-990)—sense-1 sequence (nucleotides 991-1181)—StuI restriction enzyme site (nucleotides 1182-1187); 1187 nt.

SEQ ID NO: 156. Nucleotide (DNA) sequence corresponding to the ledNbSu[Con] molecule produced from NbSu Construct 24.5, after splicing out of the two introns, not showing 5′ leader and 3′ trailer sequences resulting from in planta expression. The linking sequences that form the loops are nucleotides 196-201 and 597-602; 805 nt.

SEQ ID NO: 157. Nucleotide (DNA) sequence corresponding to the ledNbSu[Δ22] molecule produced from NbSu Construct 24.6, after splicing out of the two introns, not showing 5′ leader and 3′ trailer sequences resulting from in planta expression. The linking sequences that form the loops are nucleotides 187-192 and 588-593; 787 nt

SEQ ID NO: 158. Nucleotide (DNA) sequence corresponding to the ledNbSu[Δ22AG] molecule produced from NbSu Construct 24.7, after splicing out of the two introns, not showing 5′ leader and 3′ trailer sequences resulting from in planta expression. The linking sequences that form the loops are nucleotides 187-192 and 588-593; 787 nt.

SEQ ID NO: 159. Nucleotide (DNA) sequence corresponding to the ledNbSu[Δ22CT] molecule produced from NbSu Construct 24.8, after splicing out of the two introns, not showing 5′ leader and 3′ trailer sequences resulting from in planta expression. The linking sequences that form the loops are nucleotides 187-192 and 588-593; 787 nt.

SEQ ID NO: 160. Nucleotide sequence of a 100 nt sense sequence for use in NbSu Constructs 24.9 and 24.10 for hpNbSu[Δ22ex] and ledNbSu[Δ22ex]; 100 nt.

SEQ ID NO: 161. Nucleotide sequence of the protein coding region of a cDNA corresponding to an EPSPS gene from Amaranthus palmeri.

SEQ ID NO: 162. Nucleotide sequence of a selected region of a cDNA corresponding to the EPSPS gene from A. palmeri, for producing hairpin (Construct 25.1) precursor RNA molecules targeting that gene; 467 nt.

SEQ ID NO: 163. Nucleotide sequence of a modified sense region comprising 21 single-nucleotide deletions relative to the wild-type sequence, for producing hairpin (Construct 25.2) precursor RNA molecules targeting EPSPS gene from A. palmeri; 446 nt.

SEQ ID NO: 164. Nucleotide sequence of a modified sense region comprising 21 single-nucleotide deletions and 38 A to G or C to T substitutions relative to the wild-type sequence, for producing hairpin (Construct 25.3) precursor RNA molecules targeting EPSPS gene from A. palmeri; 446 nt.

SEQ ID NO: 165. Nucleotide sequence of the sense-1 sequence from a cDNA corresponding to the EPSPS gene from A. palmeri, for producing ledRNA (Construct 25.4) precursor RNA molecules targeting that gene; 244 nt.

SEQ ID NO: 166. Nucleotide sequence of the sense-2 sequence from a cDNA corresponding to the EPSPS gene from A. palmeri, for producing ledRNA (Construct 25.4) precursor RNA molecules targeting that gene; 223 nt.

SEQ ID NO: 167. Nucleotide sequence of the modified sense-1 sequence having 11 single-nucleotide deletions, for producing ledRNA (Construct 25.5) precursor RNA molecules having the Δ22 modification; 233 nt.

SEQ ID NO: 168. Nucleotide sequence of the modified sense-2 sequence having 10 single-nucleotide deletions, for producing ledRNA (Construct 25.5) precursor RNA molecules targeting that gene; 213 nt.

SEQ ID NO: 169. Nucleotide sequence of a modified sense-1 region comprising 11 single-nucleotide deletions and 22 A to G or C to T substitutions relative to the wild-type sequence, for producing ledEPSPS[Δ22G:U] (Construct 25.6) precursor RNA molecules targeting EPSPS gene from A. palmeri; 233 nt.

SEQ ID NO: 170. Nucleotide sequence of a modified sense-2 region comprising 11 single-nucleotide deletions and 16 A to G or C to T substitutions relative to the wild-type sequence, for producing ledEPSPS[Δ22G:U] (Construct 25.6) precursor RNA molecules targeting EPSPS gene from A. palmeri; 213 nt.

SEQ ID NO: 171. Chimeric nucleotide sequence of two selected target regions, joined together into one sequence, from a cDNA corresponding to the Ha6 gene of H. armigera, for producing hairpin (hpHa6[Con]) precursor RNA molecules targeting that gene and as a starting point for modifications. The sequence corresponds to nucleotides 372-527 and 1248-1424 of SEQ ID NO: 52 with one C to T substitution to remove a SmaI restriction site; 333 nt.

SEQ ID NO: 172. Nucleotide sequence of a modified chimeric sense sequence comprising 15 single-nucleotide deletions relative to the chimeric sequence of SEQ ID NO: 171, for producing hairpin (hpHa6[Δ22]) precursor RNA molecules having the Δ22 modification and targeting the Ha6 gene transcript of H. armigera; 318 nt.

SEQ ID NO: 173. Nucleotide sequence of a modified, chimeric sense region comprising 51 A to G and 32 C to T substitutions relative to SEQ ID NO: 171, for producing hairpin (hpHa6[GU]) precursor RNA molecules having G:U basepairs and targeting the Ha6 gene transcript of H. armigera; 333 nt.

SEQ ID NO: 174. Nucleotide sequence of a modified sense region comprising 15 single-nucleotide deletions and 83 A to G or C to T substitutions relative to SEQ ID NO: 171, for producing hairpin (hpHa6[Δ22GU]) precursor RNA molecules having both the Δ22 modification and G:U basepairs, and targeted the Ha6 gene transcript of H. armigera, 318 nt.

SEQ ID NO: 175. Nucleotide sequence of the wild-type sense-1 sequence from a cDNA for the Ha6 gene of H. armigera, corresponding to nucleotides 372-527 of SEQ ID NO: 52 with one C to T substitution relative to the wild-type, to remove a SmaI restriction site. This was used for producing ledRNA (ledHa6[Con]) precursor RNA molecules targeting the Ha6 gene transcript and was used as a starting sequence for modification; 156 nt.

SEQ ID NO: 176. Nucleotide sequence of the wild-type sense-2 sequence from a cDNA for the Ha6 gene of H. armigera, corresponding to nucleotides 1248-1424 of SEQ ID NO: 52. This was used for producing ledRNA (ledHa6[Con]) precursor RNA molecules targeting the Ha6 gene transcript and was used as a starting sequence for modification; 177 nt.

SEQ ID NO: 177. Nucleotide sequence of the modified sense-1 sequence having 7 single-nucleotide deletions, for producing ledRNA (ledHa6[Δ22]) precursor RNA molecules having the Δ22 modification; 149 nt.

SEQ ID NO: 178. Nucleotide sequence of the modified sense-2 sequence having 8 single-nucleotide deletions, for producing ledRNA (ledHa6[Δ22]) precursor RNA molecules having the Δ22 modification targeting the Ha6 gene transcript; 169 nt.

SEQ ID NO: 179. Nucleotide sequence of a modified sense-1 region comprising 21 A to G and 21 C to T substitutions relative to the wild-type sense-1 sequence, for producing ledHa6[GU] precursor RNA molecules targeting the Ha6 gene transcript of H. armigera, 156 nt.

SEQ ID NO: 180. Nucleotide sequence of a modified sense-2 region comprising 30 A to G and 11 C to T substitutions relative to the wild-type sequence, for producing ledHa6[Δ22GU] precursor RNA molecules targeting the Ha6 gene transcript of H. armigera; 177 nt.

SEQ ID NO: 181. Nucleotide sequence of a modified sense-1 region comprising 7 single-nucleotide deletions and 42 A to G or C to T substitutions relative to the wild-type sequence, for producing ledHa6[Δ22GU] precursor RNA molecules targeting the Ha6 gene transcript of H. armigera; 149 nt.

SEQ ID NO: 182. Nucleotide sequence of a modified sense-2 region comprising 8 single-nucleotide deletions and 41 A to G or C to T substitutions relative to the wild-type sequence, for producing ledHa6[Δ22GU] (Construct 26.8) precursor RNA molecules targeting the Ha6 gene transcript of H. armigera, 169 nt.

SEQ ID NO: 183. Nucleotide sequence of Construct 27.1 encoding hpNbSu[Con]-loop1 RNA, a symmetric hairpin RNA targeting the Su transcript in N. benthamiana with a 400 nt sense sequence for its loop. The order and position of the elements are: T7 promoter (nucleotides 1-17)—sense sequence for the dsRNA region (nucleotides 21-412)—sense sequence used as a loop sequence (nucleotides 413-812)—antisense sequence (nucleotides 813-1204)—SmaI restriction enzyme site (nucleotides 1205-1210); 1210 nt.

SEQ ID NO: 184. Nucleotide sequence of Construct 27.2 encoding hpNbSu[Con]-loop2 RNA, a symmetric hairpin RNA targeting the Su transcript in N. benthamiana with a chimeric 500 nt sense sequence for its loop, comprised of nucleotides 517-616 of SEQ ID NO: 127 followed by nucleotides 855-1254 of SEQ ID NO: 127. The order and position of the elements are: T7 promoter (nucleotides 1-17)—sense sequence for the dsRNA region (nucleotides 21-412)—chimeric sense sequence used as a loop sequence (nucleotides 413-912)—antisense sequence (nucleotides 913-1304)—SmaI restriction enzyme site (nucleotides 1305-1310); 1210 nt.

SEQ ID NO: 185. Nucleotide sequence of Construct 27.4 encoding hpNbSu[Δ22]-loop1 RNA, an asymmetric hairpin RNA targeting the Su transcript in N. benthamiana with a 400 nt sense sequence for its loop comprised of nucleotides 855-1254 of SEQ ID NO: 127. The order and position of the elements are: T7 promoter (nucleotides 1-17)—Δ22-modified sense sequence for the dsRNA region (nucleotides 21-394)—sense sequence used as a loop sequence (nucleotides 395-794)—antisense sequence (nucleotides 795-1186)—SmaI restriction enzyme site (nucleotides 1187-1192); 1192 nt.

SEQ ID NO: 186. Nucleotide sequence of Construct 27.5 encoding hpNbSu[Δ22]-loop2 RNA, an asymmetric hairpin RNA targeting the Su transcript in N. benthamiana with a chimeric 500 nt sense sequence for its loop. The order and position of the elements are: T7 promoter (nucleotides 1-17)—Δ22-modified sense sequence for the dsRNA region (nucleotides 21-394)—chimeric 500 nt sense sequence used as a loop sequence (nucleotides 395-894)—antisense sequence (nucleotides 895-1286)—SmaI restriction enzyme site (nucleotides 1287-1292); 1292 nt.

SEQ ID NO: 187. Nucleotide sequence of Construct 27.6 encoding hpNbSu[Δ22]-ext RNA, an asymmetric hairpin RNA with the Δ22 modification and a 3′ extension comprising a chimeric 500 nt sense sequence, targeting the Su transcript in N. benthamiana. The order and position of the elements are: T7 promoter (nucleotides 1-17)—sense sequence (nucleotides 21-394)—linker comprising a Cat-1 intron (nucleotides 395-590)—antisense sequence (nucleotides 591-982)—chimeric sense sequence (nucleotides 983-1483). 1483 nt.

SEQ ID NO: 188. Nucleotide sequence of the cDNA for the Myzus persicae hunchback gene (LOC111033178), Accession No. XM_022313819; 3845 nt. The protein coding region corresponds to nucleotides 549-2289.

SEQ ID NO: 189. Amino acid sequence of the Myzus persicae hunchback protein (LOC111033178); 580aa.

SEQ ID NO: 190. Nucleotide sequence of genetic construct encoding the hpMpHb[Con] hairpin RNA, a symmetric hairpin RNA targeting the Hb transcript in Myzus persicae with a 500 canonical basepair dsRNA region and 240 nt chimeric antisense sequence for its loop. The order and position of the elements are: T7 promoter (nucleotides 23-39)—sense sequence for the dsRNA region (nucleotides 43-542)—chimeric antisense sequence used as a loop sequence (nucleotides 543-782)—antisense sequence (nucleotides 783-1282)—SmaI restriction enzyme site (nucleotides 1294-1289); 1307 nt.

SEQ ID NO: 191. Nucleotide sequence of genetic construct encoding the hpMpHb[G:U] hairpin RNA, a symmetric hairpin RNA with G:U basepairs targeting the Hb transcript in Myzus persicae. The order and position of the elements are: T7 promoter (nucleotides 23-39)—modified sense sequence for the dsRNA region (nucleotides 43-542)—chimeric antisense sequence used as a loop sequence (nucleotides 543-782)—antisense sequence (nucleotides 783-1282)—SmaI restriction enzyme site (nucleotides 1294-1289). 1307 nt.

SEQ ID NO: 192. Nucleotide sequence of a cDNA for a Spodoptera frugiperda synaptic vesicle glycoprotein 2B transcript, variant X1, (LOC118274261; Accession No. XM_050705251; Sf1); 2640 nt. The protein coding region corresponds to nucleotides 264-1940.

SEQ ID NO: 193. Amino acid sequence of a Spodoptera frugiperda synaptic vesicle glycoprotein 2B (LOC118274261; Sf1 polypeptide), variant X1; 558aa.

SEQ ID NO: 194. Nucleotide sequence of a cDNA for a Spodoptera frugiperda v-type proton ATPase catalytic subunit A, variant X1, (LOC118267501; Accession No. XM_035581534; Sf2); 2767 nt. The protein coding region corresponds to nucleotides 170-2020.

SEQ ID NO: 195. Amino acid sequence of a Spodoptera frugiperda v-type proton ATPase catalytic subunit A, variant X1 (LOC118267501; Accession No. XM_035581534; Sf2); 616aa.

SEQ ID NO: 196. Nucleotide sequence of a cDNA for a Spodoptera frugiperda nuclear hormone receptor HR3, variant X3, (LOC118279815; Accession No. XM_050702331; Sf3); 4906 nt. The protein coding region corresponds to nucleotides 1152-3335.

SEQ ID NO: 197. Amino acid sequence of a Spodoptera frugiperda nuclear hormone receptor HR3, variant X3 (LOC118279815; Accession No. XM_050702331; Sf3); 727aa.

SEQ ID NO: 198. Nucleotide sequence of a cDNA for a Spodoptera frugiperda attacin-A-like protein, (LOC118270353; Accession No. XM_035585918; Sf4); 953 nt. The protein coding region corresponds to nucleotides 79-837.

SEQ ID NO: 199. Amino acid sequence of a Spodoptera frugiperda attacin-A-like protein, (LOC118270353; Accession No. XM_035585918; Sf4); 252aa.

SEQ ID NO: 200. Nucleotide sequence of a cDNA for a Spodoptera frugiperda ecdysone receptor, transcript variant X5 (LOC118267067; Accession No. XM_035580825; Sf5); 6500 nt. The protein coding region corresponds to nucleotides 707-2266.

SEQ ID NO: 201. Amino acid sequence of a Spodoptera frugiperda ecdysone receptor, variant X5 (LOC118267067; Accession No. XM_035580825; Sf5); 519aa.

SEQ ID NO: 202. Nucleotide sequence of a cDNA for a Spodoptera frugiperda coatomer subunit beta, transcript variant X2 (LOC118275640; Accession No. XM_035594799; Sf6); 4789 nt. The protein coding region corresponds to nucleotides 157-3006.

SEQ ID NO: 203. Amino acid sequence of a Spodoptera frugiperda coatomer subunit beta, variant X2 (LOC118275640; Accession No. XM_035594799; Sf6); 949aa.

SEQ ID NO: 204. Nucleotide sequence of a cDNA for a Spodoptera frugiperda beta-1,3-glucan-binding protein (LOC118281636; Accession No. XM_035602265.2; Sf7); 1376 nt. The protein coding region corresponds to nucleotides 155-1282.

SEQ ID NO: 205. Amino acid sequence of a Spodoptera frugiperda beta-1,3-glucan-binding protein (LOC118281636; Accession No. XM_035602265.2; Sf7); 375aa.

SEQ ID NO: 206. Nucleotide sequence of a cDNA for a Spodoptera frugiperda troponin C (LOC118272276; Accession No. XM_035588676; Sf8); 948 nt. The protein coding region corresponds to nucleotides 135-590.

SEQ ID NO: 207. Amino acid sequence of a Spodoptera frugiperda troponin C (LOC118272276; Accession No. XM_035588676; Sf8); 151aa.

SEQ ID NO: 208. Nucleotide sequence of DNA fragment encoding the hpSf1[G:U] hairpin RNA, a symmetric hairpin RNA targeting the Sf1 transcript in Spodoptera frugiperda having a 300 basepair dsRNA region with 78 C to T substitutions (26% G:U basepairs), and a 150 nt antisense sequence for the loop. The order and position of the elements are: KpnI restriction enzyme site (nucleotides 3-8)—T7 promoter (nucleotides 9-25)—antisense sequence for the dsRNA region (nucleotides 29-328)—chimeric antisense sequence used as a loop sequence (nucleotides 329-478)—modified sense sequence (nucleotides 479-778)—SpeI restriction enzyme site (nucleotides 785-790). 790 nt.

SEQ ID NO: 209. Nucleotide sequence of DNA fragment encoding the hpSf2[G:U] hairpin RNA, a symmetric hairpin RNA targeting the Sf2 transcript in Spodoptera frugiperda having a 302 basepair dsRNA region with 77 C to T substitutions (26% G:U basepairs), and a 150 nt antisense sequence for the loop. The order and position of the elements are: KpnI restriction enzyme site (nucleotides 3-8)—T7 promoter (nucleotides 9-25)—antisense sequence for the dsRNA region (nucleotides 29-330)—chimeric antisense sequence used as a loop sequence (nucleotides 331-476)—modified sense sequence (nucleotides 477-778)—SpeI restriction enzyme site (nucleotides 785-790). 790 nt.

SEQ ID NO: 210. Nucleotide sequence of DNA fragment encoding the hpSf3[G:U] hairpin RNA, a symmetric hairpin RNA targeting the Sf3 transcript in Spodoptera frugiperda having a 300 basepair dsRNA region with 79 C to T substitutions (26.3% G:U basepairs), and a 150 nt antisense sequence for the loop. The order and position of the elements are: KpnI restriction enzyme site (nucleotides 3-8)—T7 promoter (nucleotides 9-25)—antisense sequence for the dsRNA region (nucleotides 29-328) chimeric antisense sequence used as a loop sequence (nucleotides 329-478)—modified sense sequence (nucleotides 479-778)—SpeI restriction enzyme site (nucleotides 785-790). 790 nt.

SEQ ID NO: 211. Nucleotide sequence of DNA fragment encoding the hpSf4[G:U] hairpin RNA, a symmetric hairpin RNA targeting the Sf4 transcript in Spodoptera frugiperda having a 300 basepair dsRNA region with 89 C to T substitutions (29.7% G:U basepairs), and a 146 nt antisense sequence for the loop. The order and position of the elements are: KpnI restriction enzyme site (nucleotides 3-8)—T7 promoter (nucleotides 9-25)—antisense sequence for the dsRNA region (nucleotides 29-328)—chimeric antisense sequence used as a loop sequence (nucleotides 329-474)—modified sense sequence (nucleotides 475-774)—SpeI restriction enzyme site (nucleotides 781-786). 786 nt.

SEQ ID NO: 212. Nucleotide sequence of DNA fragment encoding the hpSf5[G:U] hairpin RNA, a symmetric hairpin RNA targeting the Sf5 transcript in Spodoptera frugiperda having a 300 basepair dsRNA region with 76 C to T substitutions (25.3% G:U basepairs), and a 150 nt antisense sequence for the loop. The order and position of the elements are: KpnI restriction enzyme site (nucleotides 3-8)—T7 promoter (nucleotides 9-25)—antisense sequence for the dsRNA region (nucleotides 29-328)—chimeric antisense sequence used as a loop sequence (nucleotides 329-478)—modified sense sequence (nucleotides 479-778)—SpeI restriction enzyme site (nucleotides 785-790). 790 nt.

SEQ ID NO: 213. Nucleotide sequence of DNA fragment encoding the hpSf6[G:U] hairpin RNA, a symmetric hairpin RNA targeting the Sf6 transcript in Spodoptera frugiperda having a 301 basepair dsRNA region with 78 C to T substitutions (26% G:U basepairs), and a 148 nt antisense sequence for the loop. The order and position of the elements are: KpnI restriction enzyme site (nucleotides 3-8)—T7 promoter (nucleotides 9-25)—antisense sequence for the dsRNA region (nucleotides 29-329)—chimeric antisense sequence used as a loop sequence (nucleotides 330-477)—modified sense sequence (nucleotides 478-778)—SpeI restriction enzyme site (nucleotides 785-790). 790 nt.

SEQ ID NO: 214. Nucleotide sequence of DNA fragment encoding the hpSf7[G:U] hairpin RNA, a symmetric hairpin RNA targeting the Sf7 transcript in Spodoptera frugiperda having a 300 basepair dsRNA region with 77 C to T substitutions (25.6% G:U basepairs), and a 150 nt antisense sequence for the loop. The order and position of the elements are: KpnI restriction enzyme site (nucleotides 3-8)—T7 promoter (nucleotides 9-25)—antisense sequence for the dsRNA region (nucleotides 29-329)—chimeric antisense sequence used as a loop sequence (nucleotides 330-477)—modified sense sequence (nucleotides 478-778)—SpeI restriction enzyme site (nucleotides 785-790). 790 nt.

SEQ ID NO: 215. Nucleotide sequence of DNA fragment encoding the hpSf8[G:U] hairpin RNA, a symmetric hairpin RNA targeting the Sf8 transcript in Spodoptera frugiperda having a 300 basepair dsRNA region with 72 C to T substitutions (24% G:U basepairs), and a 150 nt antisense sequence for the loop. The order and position of the elements are: KpnI restriction enzyme site (nucleotides 3-8)—T7 promoter (nucleotides 9-25)—antisense sequence for the dsRNA region (nucleotides 29-328)—chimeric antisense sequence used as a loop sequence (nucleotides 329-478)—modified sense sequence (nucleotides 479-778)—SpeI restriction enzyme site (nucleotides 785-790). 790 nt.

SEQ ID NO: 216. Nucleotide sequence of the protein coding region of a Fusarium oxysporum 1,3 beta-glucan synthase (FKS1).

SEQ ID NO: 217. Amino acid sequence of a Fks1 protein from a Fusarium oxysporum, encoded by a Fks1 gene corresponding to a cDNA having the nucleotide sequence of SEQ ID NO: 216. 1941aa.

SEQ ID NO: 218. Nucleotide sequence of the protein coding region of a Fusarium oxysporum 1,3 beta-glucan synthase (FRP1), 1581 nt.

SEQ ID NO: 219. Amino acid sequence of a Frp1 protein from a Fusarium oxysporum, encoded by a Frp1 gene corresponding to a cDNA having the nucleotide sequence of SEQ ID NO: 218. 526aa.

SEQ ID NO: 220. Nucleotide sequence of a chimeric sense sequence from cDNAs of a gene encoding Fusarium oxysporum Fks1 and Frp1 proteins. This sequence corresponds to a fusion of nucleotides 4899-5248 of SEQ ID NO: 216 to nucleotides 1090-1430 of SEQ ID NO: 218.

SEQ ID NO: 221. Nucleotide sequence of genetic construct encoding the hpFoFks1/Frp1[Con] hairpin RNA, a symmetric hairpin RNA simultaneously targeting a Fks1 transcript and a Frp1 transcript in Fusarium oxysporum with a 701 canonical basepair dsRNA region and 278 nt target-specific sequence for its loop. The order and position of the elements are: T7 promoter (nucleotides 13-29)—chimeric antisense sequence for the dsRNA region (nucleotides 30-730)—chimeric loop sequence from Fks1 and Frp1 (nucleotides 731-1008)—chimeric sense sequence (nucleotides 1009-1709)—HindIII restriction enzyme site (nucleotides 1710-1715). 1723 nt.

SEQ ID NO: 222. Nucleotide sequence of genetic construct encoding the hpFoFks1/Frp1[GU] hairpin RNA, a symmetric hairpin RNA simultaneously targeting a Fks1 transcript and a Frp1 transcript in Fusarium oxysporum with a 701 basepair dsRNA region including 194 G:U basepairs and a 278 nt target-specific sequence for its loop. The order and position of the elements are: T7 promoter (nucleotides 13-29)—chimeric antisense sequence for the dsRNA region (nucleotides 30-730)—chimeric loop sequence from Fks1 and Frp1 (nucleotides 731-1008)—chimeric sense sequence (nucleotides 1009-1709)—HindIII restriction enzyme site (nucleotides 1710-1715). 1723 nt.

SEQ ID NO: 223. Nucleotide sequence of genetic construct encoding the ledFoFks1/Frp1[Con] RNA, a symmetric ledRNA simultaneously targeting a Fks1 transcript and a Frp1 transcript in Fusarium oxysporum with a 701 canonical basepair dsRNA region and 278 nt target-specific sequence for its loop. The order and position of the elements are: T7 promoter (nucleotides 13-29) antisense sequence for the first dsRNA region targeting Fks1 (nucleotides 30-379)—first loop sequence from Fks1 (nucleotides 380-519)—sense sequence for the first dsRNA region targeting Fks1 (nucleotides 520-866)—sense sequence for the second dsRNA region targeting Frp1 (nucleotides 870-1219)—second loop sequence from Frp1 (nucleotides 1220-1359)—antisense sequence for the second dsRNA (nucleotides 1360-1709)—HindIII restriction enzyme site (nucleotides 1713-1718). 1726 nt.

SEQ ID NO: 224. Nucleotide sequence of genetic construct encoding the ledFoFks1/Frp1[GU] RNA, a symmetric ledRNA simultaneously targeting a Fks1 transcript and a Frp1 transcript in Fusarium oxysporum with two dsRNA regions, one of 350 basepairs including 102 G:U basepairs and the other of 351 basepairs with 90 G:U basepairs and each with a 140 nt target-specific sequence for its loop. The order and position of the elements are: T7 promoter (nucleotides 13-29)—antisense sequence for the first dsRNA region targeting Fks1 (nucleotides 30-379)—first loop sequence from Fks1 (nucleotides 380-519)—sense sequence including C to T substitutions for the first dsRNA region targeting Fks1 (nucleotides 520-866)—sense sequence including C to T substitutions for the second dsRNA region, targeting Frp1 (nucleotides 870-1219)—second loop sequence from Frp1 (nucleotides 1220-1359)—antisense sequence for the second dsRNA (nucleotides 1360-1709)—HindIII restriction enzyme site (nucleotides 1713-1718). 1726 nt.

SEQ ID NO: 225. Nucleotide sequence of the protein coding region of a cDNA for the gene encoding a leucine-rich repeat-containing protein from Verticillium dahliae (VdLRR1, gene designation VDAG_09119), Accession No. XM_009655421; 3267 nt. The protein coding region corresponds to nucleotides 1-3267. A target region was selected corresponded to nucleotides 1646-1945 (unmodified sense sequence).

SEQ ID NO: 226. Amino acid sequence of a Verticillium dahliae leucine-rich repeat-containing protein (VdLRR1), Accession No. XM_009655421; 1088aa.

SEQ ID NO: 227. Nucleotide sequence of the protein coding region of a cDNA for a gene encoding a leucine-rich repeat-containing protein from Verticillium dahliae (VdLLR2, gene designation VDAG_07687), Accession No. XM_009656592; 5472 nt. The protein coding region corresponds to nucleotides 1-5472. A target region was selected corresponded to nucleotides 851-1150 (unmodified sense sequence).

SEQ ID NO: 228. Amino acid sequence of a Verticillium dahliae leucine-rich repeat-containing protein (VdLLR2), Accession No. XM_009656592; 1823aa.

SEQ ID NO: 229. Nucleotide sequence of the protein coding region of a cDNA for a gene encoding Verticillium dahliae cyclopentanone 1,2-monooxygenase (VdCPM, gene designation VDAG_03943), Accession No. XM_009654027; 1668 nt. The protein coding region corresponds to nucleotides 1-1668. A target region was selected corresponded to nucleotides 161-460 (unmodified sense sequence).

SEQ ID NO: 230. Amino acid sequence of a Verticillium dahliae cyclopentanone 1,2-monooxygenase (VdCPM), Accession No. XM_009654027; 555aa.

SEQ ID NO: 231. Nucleotide sequence of the cDNA for the gene encoding Verticillium dahliae NADPH oxidase (VdNOX, gene designation VDAG_09930), Accession No. XM_009651275; 2630 nt. The protein coding region corresponds to nucleotides 610-2331. A target region was selected corresponded to nucleotides 1570-1869 (unmodified sense sequence).

SEQ ID NO: 232. Amino acid sequence of a Verticillium dahliae NADPH oxidase (VdNOX), Accession No. XM_009651275; 573aa.

SEQ ID NO: 233. Nucleotide sequence of a partial cDNA for a gene encoding Verticillium dahliae thiamine transporter (VdTTR, gene designation VDAG_03620), Accession No. XM_009653704; 1634 nt. The protein coding region corresponds to nucleotides 1-1593. A target region was selected corresponded to nucleotides 189-488 (unmodified sense sequence).

SEQ ID NO: 234. Amino acid sequence of a Verticillium dahliae thiamine transporter (VdTTR), Accession No. XM_009653704; 530aa.

SEQ ID NO: 235. Nucleotide sequence of the cDNA for the gene encoding Verticillium dahliae thiamine thiazole synthase (VdTTS, gene designation VDAG_01137), Accession No. XM_009650023; 1282 nt. The protein coding region corresponds to nucleotides 195-1151. A target region was selected corresponded to nucleotides 261-560 (unmodified sense sequence).

SEQ ID NO: 236. Amino acid sequence of the Verticillium dahliae thiamine thiazole synthase protein (VdTTS, gene designation VDAG_01137), Accession No. XM_009650023; 318aa.

SEQ ID NO: 237. Nucleotide sequence of genetic construct 31.1 encoding the hpVdLRR1[Con] hairpin RNA, a symmetric hairpin RNA targeting a LRR transcript in Verticillium dahliae with a 300 canonical basepair dsRNA region and 130 nt sequence for its loop. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 238. Nucleotide sequence of genetic construct 31.2 encoding the first hpVdLRR1[G:U] hairpin RNA, a symmetric hairpin RNA targeting a LRR transcript in Verticillium dahliae with a 300 canonical basepair dsRNA region formed by C to T substitutions, and a 130 nt sequence for its loop. The dsRNA region has 25.3% G:U basepairing. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 239. Nucleotide sequence of genetic construct 31.3 encoding the second hpVdLRR1[G:U] hairpin RNA, a symmetric hairpin RNA targeting a LRR transcript in Verticillium dahliae with a 300 canonical basepair dsRNA region formed from A to G substitutions, and 130 nt sequence for its loop. The dsRNA region has 24.3% G:U basepairing. The order and position of the elements are: T7 promoter (nucleotides 12-28)—modified sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 240. Nucleotide sequence of genetic construct 31.4 encoding the hpVdLRR1[Δ22] hairpin RNA, an asymmetric hairpin RNA targeting a LRR transcript in Verticillium dahliae with a 300-ribonucleotide antisense sequence and a 286-ribonucleotide sense sequence forming the dsRNA region, and a 130 nt sequence for its loop. The order and position of the elements are: T7 promoter (nucleotides 12-28)—sense sequence for the dsRNA region (nucleotides 29-314)—loop sequence from GUS (nucleotides 315-444)—antisense sequence (nucleotides 445-744)—SpeI restriction enzyme site (nucleotides 751-756); 763 nt.

SEQ ID NO: 241. Nucleotide sequence of genetic construct 31.5 encoding the ledVdLRR1[Con] RNA, a symmetric ledRNA targeting a LRR transcript in Verticillium dahliae with canonical basepairs in both dsRNA regions and 130 nt sequences for its loops. The order and position of the elements are: T7 promoter (nucleotides 12-28)—sense 2 sequence for one dsRNA region (nucleotides 29-178)—loop sequence from GUS (nucleotides 179-308)—antisense sequence (nucleotides 309-608)—loop sequence from GUS (nucleotides 609-738)—sense 1 sequence for the second dsRNA region (nucleotides 739-888)—SpeI restriction enzyme site (nucleotides 892-897); 904 nt.

SEQ ID NO: 242. Nucleotide sequence of genetic construct 31.6 encoding the hpVdLRR2[Con] hairpin RNA, a symmetric hairpin RNA targeting a LRR transcript (VDAG_07687) in Verticillium dahliae with a 300 canonical basepair dsRNA region and 130 nt sequence for its loop. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 243. Nucleotide sequence of genetic construct 31.7 encoding the hpVdLRR2[G:U] hairpin RNA, a symmetric hairpin RNA targeting a LRR transcript in Verticillium dahliae with a 300 canonical basepair dsRNA region formed by C to T substitutions, and a 130 nt sequence for its loop. The dsRNA region has 25.6% G:U basepairing. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 244. Nucleotide sequence of genetic construct 31.8 encoding the hpVdLRR2[Δ22] hairpin RNA, an asymmetric hairpin RNA targeting a LRR transcript in Verticillium dahliae with a 300-ribonucleotide antisense sequence and a 286-ribonucleotide sense sequence forming the dsRNA region, and a 130 nt sequence for its loop. The order and position of the elements are: T7 promoter (nucleotides 12-28)—sense sequence for the dsRNA region (nucleotides 29-314)—loop sequence from GUS (nucleotides 315-444)—antisense sequence (nucleotides 445-744)—SpeI restriction enzyme site (nucleotides 751-756); 763 nt.

SEQ ID NO: 245. Nucleotide sequence of genetic construct 31.9 encoding the hpVdCPM[Con] hairpin RNA, a symmetric hairpin RNA targeting a CPM transcript (VDAG_03943) in Verticillium dahliae with a 300 canonical basepair dsRNA region and 130 nt sequence for its loop. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 246. Nucleotide sequence of genetic construct 31.10 encoding the hpVdCPM[G:U] hairpin RNA, a symmetric hairpin RNA targeting a CPM transcript in Verticillium dahliae with a 300 canonical basepair dsRNA region formed by C to T substitutions, and a 130 nt sequence for its loop. The dsRNA region has 24.3% G:U basepairing. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 247. Nucleotide sequence of genetic construct 31.11 encoding the hpVdCPM[Δ22] hairpin RNA, an asymmetric hairpin RNA targeting a CPM transcript in Verticillium dahliae with a 300-ribonucleotide antisense sequence and a 286-ribonucleotide sense sequence forming the dsRNA region, and a 130 nt sequence for its loop. The order and position of the elements are: T7 promoter (nucleotides 12-28)—sense sequence for the dsRNA region (nucleotides 29-314)—loop sequence from GUS (nucleotides 315-444)—antisense sequence (nucleotides 445-744)—SpeI restriction enzyme site (nucleotides 751-756); 763 nt.

SEQ ID NO: 248. Nucleotide sequence of genetic construct 31.12 encoding the hpVdNox[Con] hairpin RNA, a symmetric hairpin RNA targeting a Nox transcript (VDAG_09930) in Verticillium dahliae with a 300 canonical basepair dsRNA region and 130 nt sequence for its loop. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 249. Nucleotide sequence of genetic construct 31.13 encoding the hpVdNox[G:U] hairpin RNA, a symmetric hairpin RNA targeting a Nox transcript in Verticillium dahliae with a 300 canonical basepair dsRNA region formed by C to T substitutions, and a 130 nt sequence for its loop. The dsRNA region has 24.0% G:U basepairing. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 250. Nucleotide sequence of genetic construct encoding 31.14 the hpVdNox[Δ22] hairpin RNA, an asymmetric hairpin RNA targeting a Nox transcript in Verticillium dahliae with a 300-ribonucleotide antisense sequence and a 286-ribonucleotide sense sequence forming the dsRNA region, and a 130 nt sequence for its loop. The order and position of the elements are: T7 promoter (nucleotides 12-28)—sense sequence for the dsRNA region (nucleotides 29-314)—loop sequence from GUS (nucleotides 315-444)—antisense sequence (nucleotides 445-744)—SpeI restriction enzyme site (nucleotides 751-756); 763 nt.

SEQ ID NO: 251. Nucleotide sequence of genetic construct 31.15 encoding the hpVdTTR[Con] hairpin RNA, a symmetric hairpin RNA targeting a TTR transcript (VDAG_03620) in Verticillium dahliae with a 300 canonical basepair dsRNA region and 130 nt sequence for its loop. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 252. Nucleotide sequence of genetic construct 31.16 encoding the hpVdTTR[G:U] hairpin RNA, a symmetric hairpin RNA targeting a TTR transcript in Verticillium dahliae with a 300 canonical basepair dsRNA region formed by C to T substitutions, and a 130 nt sequence for its loop. The dsRNA region has 24.3% G:U basepairing. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 253. Nucleotide sequence of genetic construct 31.17 encoding the hpVdTTR[Δ22] hairpin RNA, an asymmetric hairpin RNA targeting a TTR transcript in Verticillium dahliae with a 300-ribonucleotide antisense sequence and a 286-ribonucleotide sense sequence forming the dsRNA region, and a 130 nt sequence for its loop. The order and position of the elements are: T7 promoter (nucleotides 12-28)—sense sequence for the dsRNA region (nucleotides 29-314—loop sequence from GUS (nucleotides 315-444)—antisense sequence (nucleotides 445-744)—SpeI restriction enzyme site (nucleotides 751-756); 763 nt.

SEQ ID NO: 254. Nucleotide sequence of genetic construct 31.18 encoding the hpVdTTS[Con] hairpin RNA, a symmetric hairpin RNA targeting a TTS transcript (VDAG_01137) in Verticillium dahliae with a 300 canonical basepair dsRNA region and 130 nt sequence for its loop. The order and position of the elements are: T7 promoter (nucleotides 12-28)—sense sequence for the dsRNA region (nucleotides 29-328)—loop sequence from GUS (nucleotides 329-458)—antisense sequence (nucleotides 459-758)—SpeI restriction enzyme site (nucleotides 765-770); 777 nt.

SEQ ID NO: 255. Nucleotide sequence of genetic construct 31.19 encoding the hpVdTTS[G:U] hairpin RNA, a symmetric hairpin RNA targeting a TTS transcript in Verticillium dahliae with a 300 canonical basepair dsRNA region formed by C to T substitutions, and a 130 nt sequence for its loop. The dsRNA region has 24.3% G:U basepairing. The order and position of the elements are: T7 promoter (nucleotides 12-28)—sense sequence for the dsRNA region (nucleotides 29-328)—loop sequence from GUS (nucleotides 329-458) antisense sequence (nucleotides 459-758)—SpeI restriction enzyme site (nucleotides 765-770); 777 nt.

SEQ ID NO: 256. Nucleotide sequence of genetic construct 31.20 encoding the hpVdTTS[Δ22] hairpin RNA, an asymmetric hairpin RNA targeting a TTS transcript in Verticillium dahliae with a 300-ribonucleotide antisense sequence and a 286-ribonucleotide sense sequence forming the dsRNA region, and a 130 nt sequence for its loop. The order and position of the elements are: T7 promoter (nucleotides 12-28)—sense sequence for the dsRNA region (nucleotides 29-314)—loop sequence from GUS (nucleotides 315-444)—antisense sequence (nucleotides 445-744)—SpeI restriction enzyme site (nucleotides 751-756); 763 nt.

SEQ ID NO: 257. Nucleotide sequence of a cDNA encoding a chitin binding domain protein in Varroa destructor (VdCHIBIN; Accession No. XM_022792159.1, LOC111244764). The protein coding sequence corresponds to nucleotides 330-665; 710 nt.

SEQ ID NO: 258. Nucleotide sequence of a cDNA encoding a crustacean hyperglycaemic hormone protein in Varroa destructor (VdCHH; Accession No. XM_022802805.1, LOC111249228). The protein coding sequence corresponds to nucleotides 1-330; 1181 nt.

SEQ ID NO: 259. Nucleotide sequence of a cDNA encoding a calmodulin-like protein in Varroa destructor (VdCALL; Accession No. XM_022799184, LOC111247793). The protein coding sequence corresponds to nucleotides 787-1236; 1498 nt.

SEQ ID NO: 260. Amino acid sequence of a chitin binding domain protein in Varroa destructor (VdCHIBIN; Accession No. XP_022647894); 111aa.

SEQ ID NO: 261. Amino acid sequence of a crustacean hyperglycaemic hormone protein in Varroa destructor (VdCHH; Accession No. XP_022658540); 109aa.

SEQ ID NO: 262. Amino acid sequence of a calmodulin-like protein in Varroa destructor (VdCALL; Accession No. XP_022654919); 149aa.

SEQ ID NO: 263. Nucleotide sequence of a selected sense sequence used as a target sequence for the VdCHIBIN gene in Varroa destructor. This sequence corresponds to nucleotides 166-665 of SEQ ID NO: 257; 500 nt.

SEQ ID NO: 264. Nucleotide sequence of a selected sense sequence used as a target sequence for the VdCHH gene in Varroa destructor. This sequence corresponds to nucleotides 111-610 of SEQ ID NO: 258; 500 nt.

SEQ ID NO: 265. Nucleotide sequence of a selected sense sequence used as a target sequence for the calmodulin-like gene in Varroa destructor (VdCALL). This sequence corresponds to nucleotides 803-1302 of SEQ ID NO: 259; 500 nt.

SEQ ID NO: 266. Nucleotide sequence of RNA coding region for ledCHIBIN[Con] RNA, a symmetric ledRNA with only canonical basepairing targeting the chitin binding domain protein transcript in V. destructor. The 5′ to 3′ order and position of the elements are: T7 promoter (nucleotides 7-23); antisense sequence 1 (nucleotides 24-273); loop 1 sequence which corresponds to an Arabidopsis thaliana sequence unrelated to SEQ ID NO: 257 (nucleotides 274-423); two contiguous sense sequences corresponding to the target sequence (nucleotides 424-923), loop 2 sequence which corresponds to the A. thaliana sequence (nucleotides 924-1073); and antisense sequence 2 (nucleotides 1074-1320); 1337 nt.

SEQ ID NO: 267. Nucleotide sequence of RNA coding region encoding ledCHIBIN[G:U] RNA, a symmetric ledRNA with G:U basepairs targeting the chitin binding domain protein transcript in V. destructor. The 5′ to 3′ order and position of the elements are: T7 promoter (nucleotides 7-23); antisense sequence 1 (nucleotides 24-273); loop 1 sequence which corresponds to an Arabidopsis thaliana sequence unrelated to SEQ ID NO: 257 (nucleotides 274-423); sense sequence corresponding to the target sequence with 106 C to T substitutions (21.2%) (nucleotides 424-923), loop 2 sequence which corresponds to the A. thaliana sequence (nucleotides 924-1073); and antisense sequence 2 (nucleotides 1074-1320); 1337 nt.

SEQ ID NO: 268. Nucleotide sequence of RNA coding region for ledCHH[Con] RNA, a symmetric ledRNA with only canonical basepairing targeting the CHH transcript in V. destructor. The 5′ to 3′ order and position of the elements are: T7 promoter (nucleotides 7-23); antisense sequence 1 (nucleotides 24-273); loop 1 sequence which corresponds to an Arabidopsis thaliana sequence unrelated to SEQ ID NO: 257 (nucleotides 274-423); two contiguous sense sequences corresponding to the target sequence (nucleotides 424-923), loop 2 sequence which corresponds to the A. thaliana sequence (nucleotides 924-1073); and antisense sequence 2 (nucleotides 1074-1320); 1337 nt.

SEQ ID NO: 269. Nucleotide sequence of RNA coding region encoding ledCHH[G:U] RNA, a symmetric ledRNA with G:U basepairs targeting the CHH transcript in V. destructor. The 5′ to 3′ order and position of the elements are: T7 promoter (nucleotides 7-23); antisense sequence 1 (nucleotides 24-273); loop 1 sequence which corresponds to an Arabidopsis thaliana sequence unrelated to SEQ ID NO: 257 (nucleotides 274-423); sense sequence corresponding to the target sequence with 100 C to T substitutions (20%) (nucleotides 424-923), loop 2 sequence which corresponds to the A. thaliana sequence (nucleotides 924-1073); and antisense sequence 2 (nucleotides 1074-1320); 1337 nt.

SEQ ID NO: 270. Nucleotide sequence of a construct encoding ledCALL[Con] RNA molecules, a symmetric ledRNA targeting the calmodulin-like transcript in V. destructor. The 5′ to 3′ order and position of the elements are: T7 promoter (nucleotides 7-23); antisense sequence 1 which corresponds to the complement of nucleotides 803-1052 of SEQ ID NO: 259 (nucleotides 24-273); loop 1 sequence which corresponds to an Arabidopsis thaliana sequence unrelated to SEQ ID NO: 257 (nucleotides 274-423); sense sequence corresponding to the target sequence, nucleotides 803-1302 of SEQ ID NO: 259 (nucleotides 424-923), loop 2 sequence which corresponds to the A. thaliana sequence (nucleotides 924-1073); and antisense sequence 2 which corresponds to the complement of nucleotides 1056-1302 of SEQ ID NO: 259 (nucleotides 1074-1320); 1337 nt.

SEQ ID NO: 271. Nucleotide sequence of a construct encoding ledCALL[G:U] RNA molecules, a symmetric ledRNA with G:U basepairs targeting the calmodulin-like transcript (VdCALL) in V. destructor. The 5′ to 3′ order and position of the elements are: T7 promoter (nucleotides 7-23); antisense sequence 1 which corresponds to the complement of nucleotides 803-1052 of SEQ ID NO: 259 (nucleotides 24-273); loop 1 sequence which corresponds to the complement of nucleotides 653-802 of SEQ ID NO: 259 (nucleotides 274-423); sense sequence corresponding to the target sequence with 107 C to T substitutions (21.2%) (nucleotides 424-923), loop 2 sequence which corresponds to the complement of nucleotides 1303-1452 of SEQ ID NO: 259 (nucleotides 924-1073); and antisense sequence 2 which corresponds to the complement of nucleotides 1056-1302 of SEQ ID NO: 259 (nucleotides 1074-1320); 1337 nt.

SEQ ID NO: 272. Nucleotide sequence of a construct encoding ledCALL[Δ22] RNA molecules, an asymmetric ledRNA targeting the calmodulin-like transcript in V. destructor. The 5′ to 3′ order and position of the elements are: T7 promoter (nucleotides 7-23); antisense sequence 1 which corresponds to the complement of nucleotides 803-1052 of SEQ ID NO: 259 (nucleotides 24-273); loop 1 sequence which corresponds to the complement of nucleotides 653-802 of SEQ ID NO: 259 (nucleotides 274-423); sense sequence corresponding to the target sequence except that every 22^{nd} nucleotide was deleted (nucleotides 424-900), loop 2 sequence which corresponds to the complement of nucleotides 1303-1452 of SEQ ID NO: 259 (nucleotides 901-1050); and antisense sequence 2 which corresponds to the complement of nucleotides 1056-1302 of SEQ ID NO: 259 (nucleotides 1051-1297); 1314 nt.

SEQ ID NO: 273. Nucleotide sequence of a construct encoding ledCALL[Δ22G:U] RNA molecules, an asymmetric ledRNA with the Δ22 modification and G:U basepairs in both dsRNA regions, targeting the calmodulin-like transcript (VdCALL) in V. destructor. The 5′ to 3′ order and position of the elements are: T7 promoter (nucleotides 7-23); antisense sequence 1 which corresponds to the complement of nucleotides 803-1052 of SEQ ID NO: 259 (nucleotides 24-273); loop 1 sequence which corresponds to the complement of nucleotides 653-802 of SEQ ID NO: 259 (nucleotides 274-423); sense sequence corresponding to the target sequence with the Δ22 modification and 107 C to T substitutions (21.2%) (nucleotides 424-900), loop 2 sequence which corresponds to the complement of nucleotides 1303-1452 of SEQ ID NO: 259 (nucleotides 901-1050); and antisense sequence 2 which corresponds to the complement of nucleotides 1056-1302 of SEQ ID NO: 259 (nucleotides 1051-1297); 1314 nt.

SEQ ID NO: 274. Nucleotide sequence of a cDNA for a gene encoding an acetolactate synthase (ALS) in Raphanus raphanistrum (RrALS). The protein coding sequence corresponds to nucleotides 1-1386; 1399 nt.

SEQ ID NO: 275. Nucleotide sequence of a cDNA for a gene encoding 15-cis-phytoene desaturase (PDS1) in Raphanus raphanistrum (RrPDS1). The protein coding sequence corresponds to nucleotides 1-1422; 1422 nt.

SEQ ID NO: 276. Nucleotide sequence of a cDNA for a gene encoding 15-cis-phytoene desaturase (PDS2) in Raphanus raphanistrum (RrPDS2). The protein coding sequence corresponds to nucleotides 1-1701; 1701 nt.

SEQ ID NO: 277. Nucleotide sequence of a cDNA for a gene encoding 5-enolpyruvylshikimate-3-phosphate synthase (EPSPS) in Raphanus raphanistrum (RrEPSPS). The protein coding sequence corresponds to nucleotides 1-1359; 1359 nt.

SEQ ID NO: 278. Nucleotide sequence of a cDNA for a gene encoding filamentation temperature-sensitive H-1 (FtSH1) in Raphanus raphanistrum (RrFtSH1). The protein coding sequence corresponds to nucleotides 1-1869; 1869 nt.

SEQ ID NO: 279. Amino acid sequence of an acetolactate synthase (ALS) in Raphanus raphanistrum (RrALS); 461aa.

SEQ ID NO: 280. Amino acid sequence of PDS1 in Raphanus raphanistrum (RrPDS1); 474aa.

SEQ ID NO: 281. Amino acid sequence of PDS2 in Raphanus raphanistrum (RrPDS2); 566aa.

SEQ ID NO: 282. Amino acid sequence of an EPSPS in Raphanus raphanistrum (RrEPSPS); 452aa.

SEQ ID NO: 283. Amino acid sequence of FtSH1 in Raphanus raphanistrum (RrFtSH1); 622aa.

SEQ ID NO: 284. Nucleotide sequence of a selected sense sequence used as a target sequence for the RrEPSPS gene in Raphanus raphanistrum. This sequence corresponds to nucleotides 124-598 of SEQ ID NO: 277; 475 nt.

SEQ ID NO: 285. Nucleotide sequence encoding ledRrEPSPS[Δ22] RNA, an asymmetric ledRNA with the Δ22 modification targeting the mRNA encoding EPSPS in Raphanus raphanistrum. The 5′ to 3′ order and position of the elements are: T7 promoter (nucleotides 7-23); antisense sequence 1 complementary to nucleotides 124-376 of SEQ ID NO: 277 (nucleotides 24-276); loop 1 sequence which is a sense sequence which corresponds to nucleotides 4-123 of SEQ ID NO: 277 (nucleotides 277-396); sense sequence corresponding to the target sequence but with the Δ22 modification (nucleotides 424-923), loop 2 sequence which is a sense sequence which corresponds to nucleotides 599-717 of SEQ ID NO: 277 (nucleotides 850-969); and antisense sequence 2 complementary to nucleotides 376-598 of SEQ ID NO: 277 (nucleotides 970-1192); 1192 nt.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in gene silencing, cell culture, molecular genetics, protein chemistry, and biochemistry).

Unless otherwise indicated, the recombinant protein, cell culture, and immunological techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In double-stranded RNA-mediated post-transcriptional gene silencing (PTGS), a double-stranded RNA (dsRNA) molecule is processed by a Dicer or Dicer-like protein of the RNase III class into small double-stranded RNAs known as short interfering RNAs (siRNAs) which typically range from 21-24 ribonucleotides in length. One RNA strand from the siRNA, with an Argonaute protein (AGO), is incorporated into an RNA-induced silencing complex (RISC), where that RNA strand guides the RISC complex to cleavage or inhibition of translation of a target RNA molecule through hybridisation between the guide RNA and the target RNA molecule. sRNAs of 24 ribonucleotides can also function in plants for methylation of C nucleotides in the nuclear DNA. PTGS therefore relies on an antisense sRNA for the reduction of target RNA activity.

The term “antisense ribonucleic acid sequence” or “antisense RNA sequence”, or variations thereof, as used herein means an RNA sequence that is at least partially complementary to at least a part of a target RNA molecule to which it hybridizes. In certain embodiments, an antisense RNA sequence has antisense activity in that it decreases the expression or amount of a target RNA molecule or its activity, for example through reducing translation of the target RNA molecule. In certain embodiments, an antisense RNA sequence alters splicing of a target pre-mRNA resulting in a different splice variant. Exemplary components of antisense sequences include, but are not limited to, oligonucleotides of 21-24 ribonucleotides, and longer antisense RNA molecules of 25-33 ribonucleotides, 34-50 ribonucleotides or more than 50 or more than 100 ribonucleotides, to a maximum of the full length of a target RNA molecule.

The term “ribonucleotide” as used herein means a nucleotide that comprises ribose as its pentose component, having a 2′ hydroxyl group on the ribose. A ribonucleotide therefore has a base which is adenine (A), cytosine (C), guanosine (G) or uracil (U), covalently linked to the ribose which in turn is linked to a phosphate group (PO₄) by a phosphodiester bond when it is incorporated into an RNA molecule. Thymine (T) does rarely occur naturally in RNA molecules, as do some other naturally occurring variant bases. As used herein, an “RNA molecule” is a polymer of ribonucleotides which are covalently linked in a 5′ to 3′ orientation as is well known in the art, referring to the phosphodiester bonds of a phosphate to the 5′ hydroxyl of the ribose group and the phosphate of the adjacent ribonucleotide bonded to the 3′ hydroxyl group. The RNA molecule may contain zero, one, two or three phosphate groups at the 5′ terminus of the polyribonucleotide chain. The RNA molecule may be 2′-O-methylated at the 3′ terminal ribonucleotide. Aside from these groups, the term “ribonucleotide” as used herein excludes nucleotides with a deoxyribose group, or modified or substituted 2′ hydroxyl groups on the ribose group such as 2′O-methyl or 2′-fluoro, or sulphur atoms forming thioester bonds. The RNA molecule may have non-ribonucleotide groups attached to the 5′ and/or 3′ termini such as lipid groups of polypeptides.

The term “silencing activity” or “antisense activity” is used in the context of the present disclosure to refer to any detectable and/or measurable activity attributable to the hybridisation of an antisense RNA sequence to its target RNA molecule. Such detection and/or measuring may be direct or indirect. For example, silencing or antisense activity is assessed by detecting and/or measuring the amount of target RNA molecule transcript. Silencing or antisense activity may also be detected as a change in a phenotype associated with the target RNA molecule. As used herein, the term “target RNA molecule” refers to an RNA molecule that is inhibited at least partially by an antisense RNA sequence according to the present disclosure. Accordingly, “target RNA molecule” can be any RNA molecule the expression or activity of which is capable of being reduced by an antisense RNA sequence. Exemplary target RNA molecules include, but are not limited to, RNA (including, but not limited to pre-mRNA and mRNA or portions thereof) transcribed from DNA encoding a target protein, rRNA, tRNA, small nuclear RNA, and miRNA, including their precursor forms. The target RNA may be the genomic RNA of a pest such as an insect pest or a nematode, or a pathogen such as a fungal pathogen or virus, or an RNA molecule derived therefrom such as a replicative form of a viral pathogen, or transcript therefrom. For example, the target RNA molecule can be an RNA from an endogenous gene such as a mRNA transcribed from the gene or a gene which is introduced or may be introduced into the eukaryotic cell whose expression is associated with a particular phenotype, trait, disorder or disease state, or a nucleic acid molecule from an infectious agent. In an example, the target RNA molecule is in a eukaryotic cell. In another example, the target RNA molecule encodes a protein. In this context, antisense activity can be assessed by detecting and or measuring the amount of target protein, for example through its activity such as enzyme activity, or a function other than as an enzyme, or through a phenotype associated with its function. As used herein, the term “target protein” refers to a protein that is modulated by an antisense RNA sequence according to the present disclosure.

Antisense activity can be detected or measured using various methods. In certain embodiments, antisense activity is assessed by detecting and/or measuring the amount of target RNA molecules and/or cleaved target RNA molecules and/or alternatively spliced target RNA molecules. In an example, antisense activity can be detected or assessed by comparing activity in a particular sample and comparing the activity to that of a control sample.

The term “targeting” is used in the context of the present disclosure to refer to the association of an antisense RNA sequence to a particular target RNA molecule or a particular region of nucleotides within a target RNA molecule, or multiple target RNA molecules such as, for example, a family of related target RNA molecules. In an example, an antisense RNA sequence according to the present disclosure shares complementarity with at least a region of a target RNA molecule. In this context, the term “complementarity” refers to a sequence of ribonucleotides that is capable of basepairing with a sequence of ribonucleotides on a target RNA molecule, through hydrogen bonding between bases on the ribonucleotides. For example, in RNA, adenine (A) is complementary to uracil (U) and guanine (G) to cytosine (C) by canonical (Watson-Crick) basepairing or through non-canonical basepairs such as G basepairing with U.

The term “observing” in the context of observing a cell or organism for phenotype or function means to visually inspect, assay, or otherwise analyse, for the purpose of identifying, recording, measuring, or determining a phenotype of the cell or organism, or function of any related molecule or compound.

As used herein, “complementary base” refers to a ribonucleotide of an antisense RNA sequence that is capable of basepairing with a ribonucleotide of a sense RNA sequence in an RNA molecule of the invention or of its target RNA molecule. For example, if a ribonucleotide at a certain position of an antisense RNA sequence is capable of hydrogen bonding with a ribonucleotide at a certain position of a target RNA molecule, then the position of hydrogen bonding between the antisense RNA sequence and the target RNA molecule is considered to be complementary at that ribonucleotide. In contrast, the term “non-complementary” or “non-basepaired” or “non-matching” refers to a pair of ribonucleotides that do not form hydrogen bonds with one another or otherwise support hybridisation. The term “complementary” with regard to a sequence rather than a ribonucleotide refers to the capacity of an antisense RNA sequence to hybridise to another nucleic acid through complementarity. In certain embodiments, an RNA sequence and its target RNA molecule are complementary to each other when a sufficient number of corresponding positions in each molecule are occupied by ribonucleotides that can bond with each other to allow stable association between the antisense RNA sequence and a sense RNA sequence in the target RNA molecule.

One of skill in the art would appreciate that various ribonucleotide combinations can basepair. Both canonical and non-canonical basepairings are contemplated by the present disclosure. In an example, a basepairing can comprise A:T or G:C in a DNA molecule or U:A or G:C in an RNA molecule. In another example, a basepairing may comprise A:G or G:T in a DNA molecule or U:G in an RNA molecule.

The term “canonical base pairing” as used in the present disclosure means basepairing between two nucleotides which are A:T or G:C for deoxyribonucleotides or A:U or G:C for ribonucleotides.

The term “non-canonical basepairing” as used in the present disclosure means an interaction between the bases of two nucleotides other than canonical basepairings, in the context of two DNA or two RNA sequences. For example, non-canonical basepairing includes pairing between G and U (G:U) or between A and G (A:G). Examples of non-canonical basepairing include purine—purine or pyrimidine—pyrimidine interactions by hydrogen bonding between the bases, as known in the art. Most commonly in the context of this disclosure, the non-canonical basepairing is G:U, preferably exclusively G:U. The G of the G:U basepair may be in either of the two RNA sequences that hybridise, or the sense RNA sequence and the antisense RNA sequence of the dsRNA region may both comprise G ribonucleotides that are involved in G:U basepairs, G in the one sequence opposite a U in the other sequence. Preferably, there are more such G ribonucleotides in the sense RNA sequence, or the corresponding region of the target RNA molecule, than in the antisense RNA sequence. Other examples of non-canonical basepairs, less preferred, in the context of extended dsRNA regions, are A: C, G: G and A: A, where those weaker base interactions must be immediately flanked on both sides by canonical basepairs.

The present disclosure refers to RNA components that “hybridise” across a series of ribonucleotides. Those of skill in the art will appreciate that terms such as “hybridise” and “hybridising” are used to describe molecules that anneal based on complementary nucleic acid sequences. Such molecules need not be 100% complementary in order to hybridise (i.e. they need not “fully basepair”), unless otherwise specified, although 100% complementarity is preferred. For example, there may be one or more mismatches in sequence complementarity. In an example, RNA components defined herein hybridise under physiological conditions such as, for example, in a eukaryotic cell. Hybridisation conditions including factors such as temperature, salt concentrations and length of sequences are well understood in the art. In an example, RNA components defined herein hybridise under stringent hybridisation conditions. The term “stringent hybridisation conditions” refers to parameters with which the art is familiar, including the variation of the hybridisation temperature with length of an RNA molecule. Ribonucleotide hybridisation parameters may be found in references which compile such methods, for example Sambrook, et al. (supra), and Ausubel, et al. (supra). For example, stringent hybridisation conditions, as used herein, can refer to hybridisation in vitro at 65° C. in hybridisation buffer (3.5×SSC, 0.02% Ficoll, 0.02% polyvinyl pyrrolidone, 0.02% Bovine Serum Albumin (BSA), 2.5 mM NaH₂PO₄ (pH7), 0.5% SDS, 2 mM EDTA), followed by one or more washes in 0.2×SSC, 0.01% BSA at 50° C. Shorter RNA components such as RNA sequences of 20-24 nucleotides in length hybridise under lower stringency conditions. The term “low stringency hybridisation conditions” refers to parameters with which the art is familiar, including the variation of the hybridisation temperature with length of an RNA molecule. For example, low stringency hybridisation conditions, as used herein, can refer to hybridisation in vitro at 42° C. in hybridisation buffer (3.5×SSC, 0.02% Ficoll, 0.02% polyvinyl pyrrolidone, 0.02% Bovine Serum Albumin (BSA), 2.5 mM NaH₂PO₄ (pH7), 0.5% SDS, 2 mM EDTA), followed by one or more washes in 0.2×SSC, 0.01% BSA at 30° C.

In the context of the present disclosure, the term “hybridisation” means the pairing of complementary polynucleotides through basepairing of complementary bases. While not limited to a particular mechanism, the most common mechanism of pairing involves hydrogen bonding, which may be Watson-Crick hydrogen bonding, between complementary ribonucleotides A:T and G:C in DNA molecules and A:U and G:C in RNA molecules, or non-canonical basepairing, mainly or exclusively G:U, in RNA molecules.

The term “covalently linked” is used in the context of the present disclosure to refer to the link between two ribonucleotide sequences as described herein such as “the first RNA strand (D) and second RNA strand (F) are covalently linked by a linking RNA sequence (L)”. As one of skill in the art would appreciate, a covalent link or bond is a chemical bond that involves the sharing of electron pairs between atoms typically via phosphodiester bonds. As used herein, two ribonucleotide sequences are “directly covalently linked” when there is a covalent bond between 3′ end of one sequence and the 5′ end of the second sequence, without any intervening ribonucleotides. Alternatively, as used herein “covalently linked by a linking RNA sequence (L)” means, for example, that there is a linking RNA sequence (L) between the first RNA strand (D) and second RNA strand (F), where (D), (L) and (F) are all linked as a single RNA strand. In this context, 5′ to 3′ order is either (D)-(L)-(F) or (F)-(L)-(D).

As used herein, the phrase “the RNA molecule has a deleterious effect on the non-human organism” or similar phrases means that the target RNA molecule is present in the non-human organism and exposure of cells expressing the target RNA molecule to the RNA molecule of the invention results in reduced levels and/or activity of the target RNA molecule when compared to the same cells lacking the RNA molecule of the invention. In an embodiment, the target RNA molecule encodes a protein important for any one or more of growth, development, feeding, movement, perception, hormone function, reproduction or survival. As an example, if the non-human organism is a crop pest or pathogen, or a pest or pathogen of an animal, the RNA molecule of the invention has a deleterious effect on one or more of feeding by the pest or pathogen, cell apoptosis, cell differentiation and development, capacity or desire for sexual reproduction, muscle formation, muscle twitching, muscle contraction, juvenile hormone formation, juvenile hormone regulation, ion regulation and transport, maintenance of cell membrane potential, amino acid biosynthesis, amino acid degradation, sperm formation, pheromone synthesis, pheromone sensing, antennae formation, wing formation, leg formation, egg formation, larval maturation, digestive enzyme formation, haemolymph synthesis, haemolymph maintenance, neurotransmission, larval stage transition, pupation, emergence from pupation, cell division, energy metabolism, respiration, chitin metabolism, and formation of cytoskeletal structure. In another example, the non-human organism is a plant which is a weed and the RNA molecule has a deleterious effect on amino acid biosynthesis, photosynthesis, fatty acid synthesis, cell membrane integrity, pigment synthesis, hormone function or growth.

As used herein, the phrase “the double-stranded RNA molecule has a beneficial effect on at least one symptom of the disease” or similar phrases means that the target RNA of the RNA molecule of the invention is present in the subject, such as for example, a plant, and exposure of cells expressing the target RNA to the RNA molecule of the invention results in reduced levels and/or activity of the target RNA when compared to the same cells lacking the RNA molecule of the invention. As used herein, the phrase “the double-stranded RNA molecule has an effect on reducing the incidence of, or preventing, a disease” means that when the target RNA of the RNA molecule of the invention is present in the subject, the presence of the RNA molecule of the invention results in reduced levels and/or activity of the target RNA when compared to the same cells lacking the RNA molecule of the invention. In an embodiment, where the subject is an animal such as, for example, a vertebrate animal, a mammalian animal such as a non-human mammal, or a human, the target RNA encodes a protein which plays a role in the presence of the disease. Alternately, the target RNA may be a small RNA such as a miRNA that plays a role in the presence of disease. In an embodiment, the disease is cancer or cancerous disease, an infectious disease, a cardiovascular disease, a neurological disease, a prion disease, an inflammatory disease, an autoimmune disease, a pulmonary disease, a renal disease, liver disease, mitochondrial disease, endocrine disease, reproduction related diseases and conditions, and any other indications that can respond to the level of an expressed gene product in a cell or organism. Target genes involved in such diseases are well known in the art.

RNA molecules according to the present disclosure and compositions comprising the same can be administered to a subject. Terms such as “subject”, “patient” or “individual” are terms that can, in context, be used interchangeably in the present disclosure. In an example, the subject is a mammal. The mammal may be a companion animal such as a dog or cat, a horse, or a livestock animal such as a sheep or cow. In one example, the subject is a human. For example, the subject can be an adult. In another example, the subject can be a child. In another example, the subject can be an adolescent. In another example, RNA molecules according to the present disclosure and compositions comprising the same can be administered to an insect. In another example, RNA molecules according to the present disclosure and compositions comprising the same can be administered to a plant, or a pathogen or pest of a plant. In another example, RNA molecules according to the present disclosure and compositions comprising the same can be administered to a fungal cell or population.

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, or +/−5%, or more preferably +/−1%, of the designated value.

### Asymmetric RNA Molecules and Precursors Thereof

As used herein, an asymmetric RNA molecule refers to a double-stranded RNA (dsRNA) molecule which comprises at least one bulge, or an RNA molecule comprising at least one double-stranded region (dsRNA region) which comprises at least one bulge, where the bulge comprises at least one non-basepaired ribonucleotide in the antisense sequence (antisense strand) of the double-stranded RNA molecule or dsRNA region which is not opposite a non-basepaired ribonucleotide in the sense sequence in the dsRNA region. In a preferred embodiment, the presence of the at least one non-basepaired ribonucleotide in the antisense sequence has the consequence that the antisense sequence of the dsRNA region is longer than the sense sequence to which it hybridises. The antisense sequence of the dsRNA region may be longer than the hybridising sense sequence by reason of having more non-basepaired ribonucleotide bulges than the sense sequence. Alternatively, the presence of the at least one bulged ribonucleotide in the antisense sequence may be offset by one or more inserted ribonucleotides in the sense sequence relative to the sequence of the corresponding region of the target RNA molecule and/or one or more deleted ribonucleotides elsewhere in the antisense sequence, so that the sense sequence is equal in length to the antisense sequence in the dsRNA region or even longer than the antisense sequence. Such molecules are included in the definition of “asymmetric RNA molecule” provided they have one or more non-basepaired ribonucleotide bulges in the antisense sequence (antisense strand) of the double-stranded RNA region which is not opposite a non-basepaired ribonucleotide in the sense sequence in the dsRNA region. In this context, the sense sequence of the dsRNA region is the sequence that has greater sequence identity to a region of a target RNA molecule, whereas the antisense sequence has greater sequence identity to the complement of the region of the target RNA molecule and thereby is intended to hybridise to the target RNA molecule. Those of skill in the art can readily identify which is the sense sequence and which is the antisense sequence in a dsRNA by the degree of identity to a region of a target RNA molecule (sense) or to its complement (antisense), readily determined by sequence alignment tools. Basepairing in the dsRNA region, aside from the non-basepaired ribonucleotides that form bulges in the dsRNA region, need not be complete between the sense and antisense sequences.

The asymmetric RNA molecule of the invention has at least one dsRNA region comprising at least one non-basepaired ribonucleotide in the antisense sequence, or more than one dsRNA region, but may also have one or more single-stranded regions; i.e. it is not necessarily fully double-stranded, although it may be fully double-stranded aside from the at least one bulging ribonucleotide(s). The dsRNA region(s) of the asymmetric RNA molecule preferably comprises multiple bulges, or preferably multiple non-basepaired ribonucleotides in the antisense sequence which are not opposite a non-basepaired ribonucleotide in the sense sequence in the dsRNA region. More preferably, the dsRNA region comprises regularly spaced bulges, also referred to as “periodic bulges”, in the antisense sequence along the length of the dsRNA region, for dsRNA regions longer than 44 basepairs. In an embodiment lacking compensatory ribonucleotide insertions into the sense sequence, the sense sequence of the dsRNA region has a shorter length than the antisense sequence, at least in part due to the one or more bulges in the antisense sequence. Where the sense sequence is the same length as the antisense sequence, or the sense sequence is longer than the antisense sequence, that is due to the presence of insertions into the sense sequence or deletions elsewhere in the antisense sequence. In a preferred embodiment, the sense sequence of the dsRNA region of the RNA molecule is shorter than the antisense sequence. Preferably, the sense sequence is shorter than the antisense sequence entirely because of the presence of the bulge(s), excluding deletions of 4 or more contiguous ribonucleotides in the sense sequence. In a more preferred embodiment, the sense sequence has a length which is between 87% and 97%, or 87% and 96%, of the length of the antisense sequence, for example the length of the sense sequence is about 21/22, 21/23 or 21/24 of the length of the antisense sequence, calculated as a fraction. In even more preferred embodiments, the sense sequence has a length which is between 91%-97% or 91-96%, or more preferably 94-97% or 94-96% of the length of the antisense sequence. Where the sense sequence has a combination of the Δ22, Δ23 and/or Δ24 modifications (as described herein), the length of the sense sequence may be intermediate between the 21/22 and 21/24 fractions relative to the antisense sequence. Again in this context, the comparison of the lengths of the sense and antisense sequences are determined along the full length of the dsRNA region.

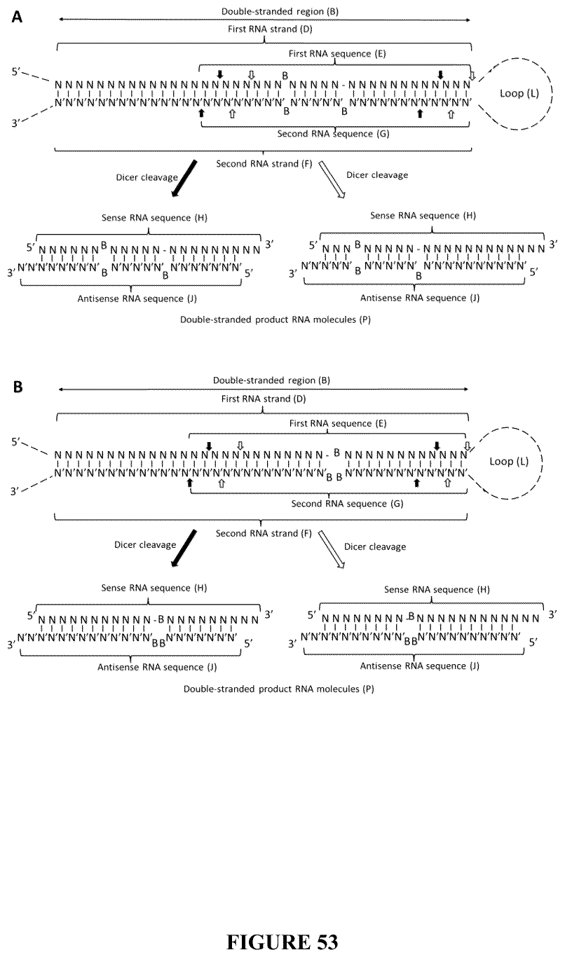

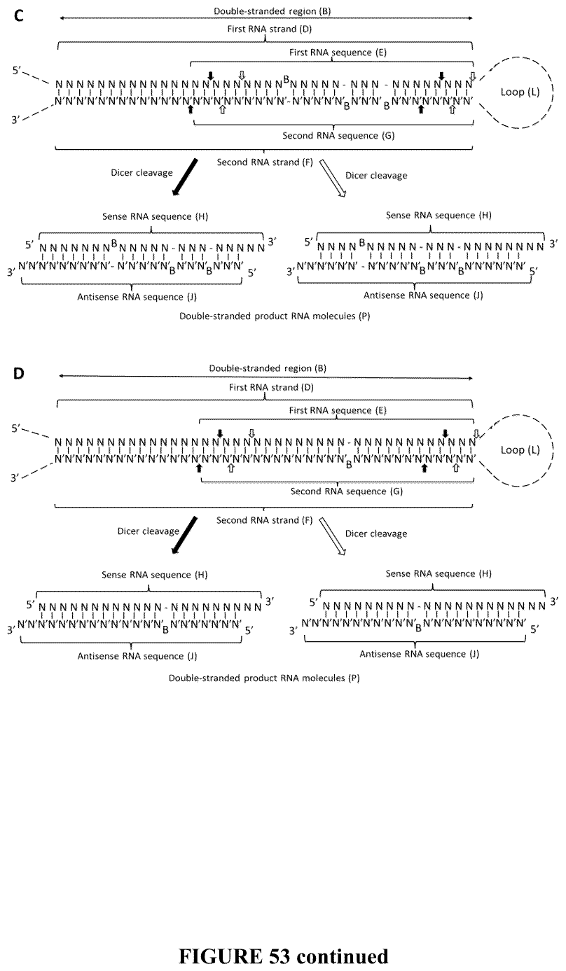

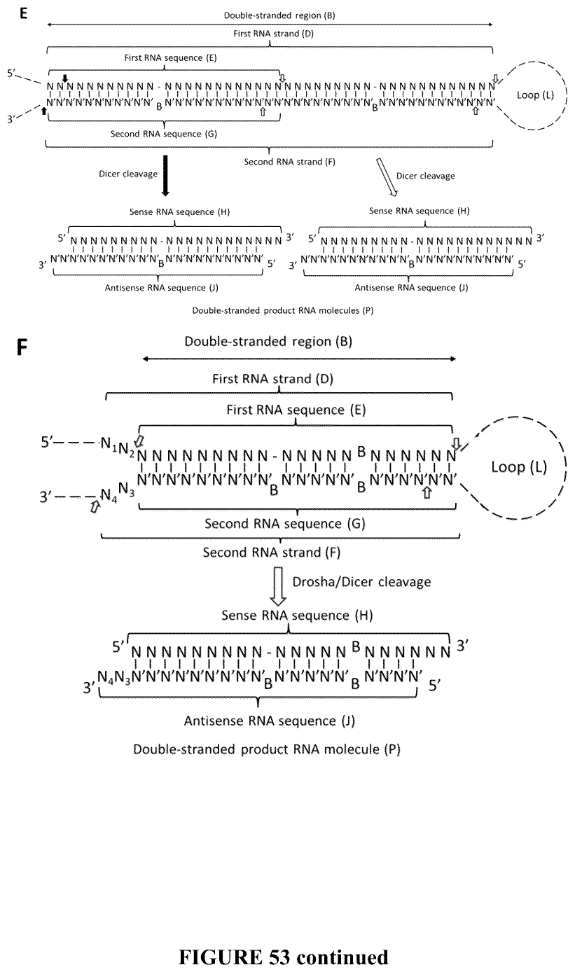

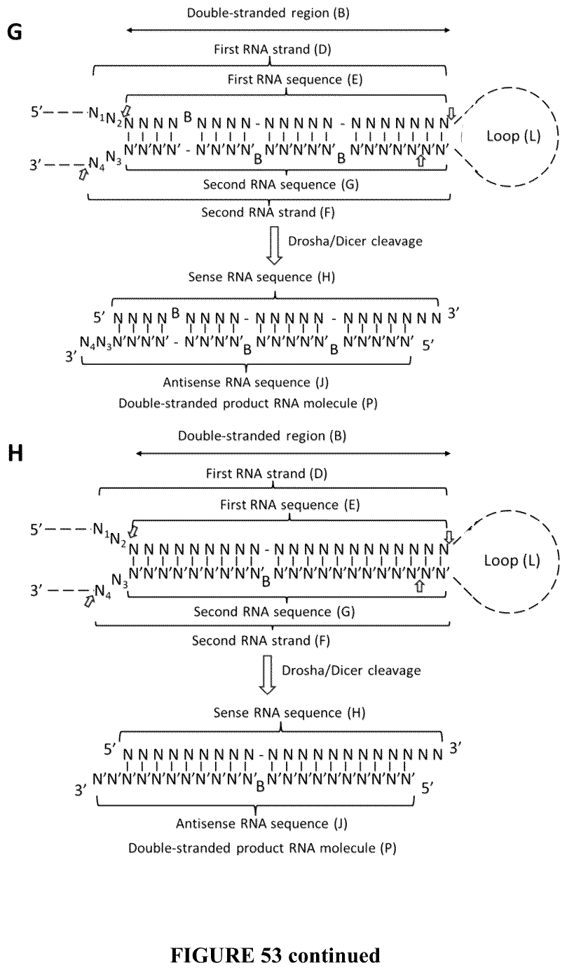

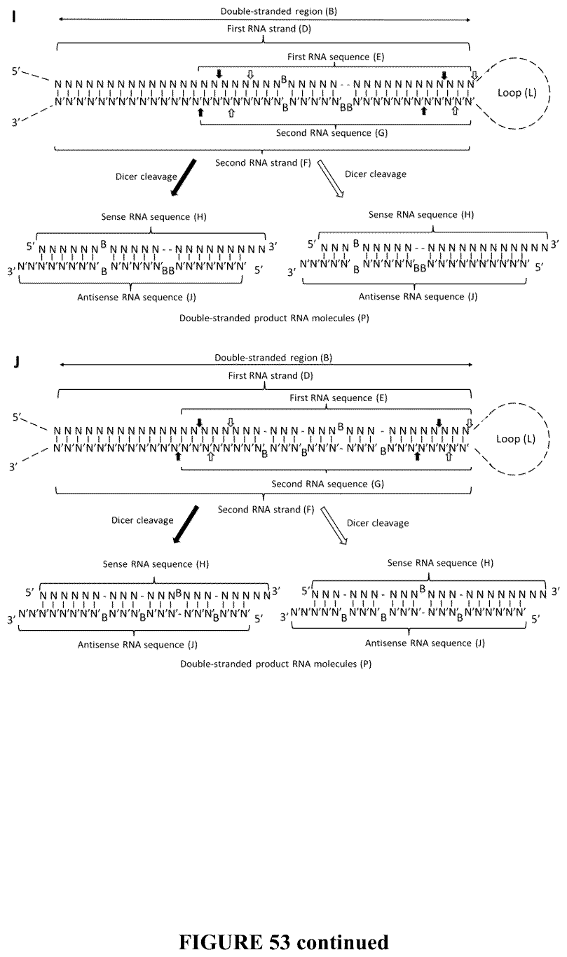

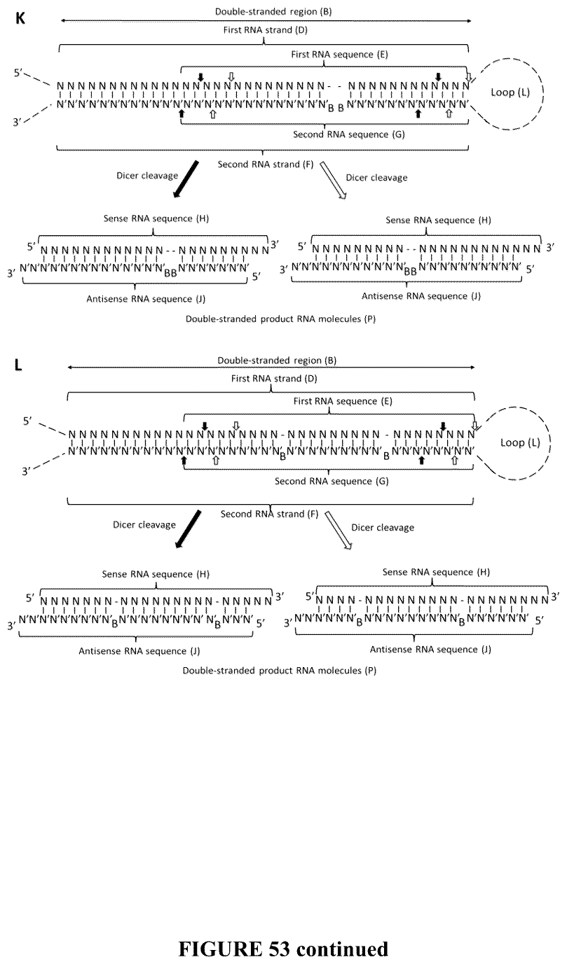

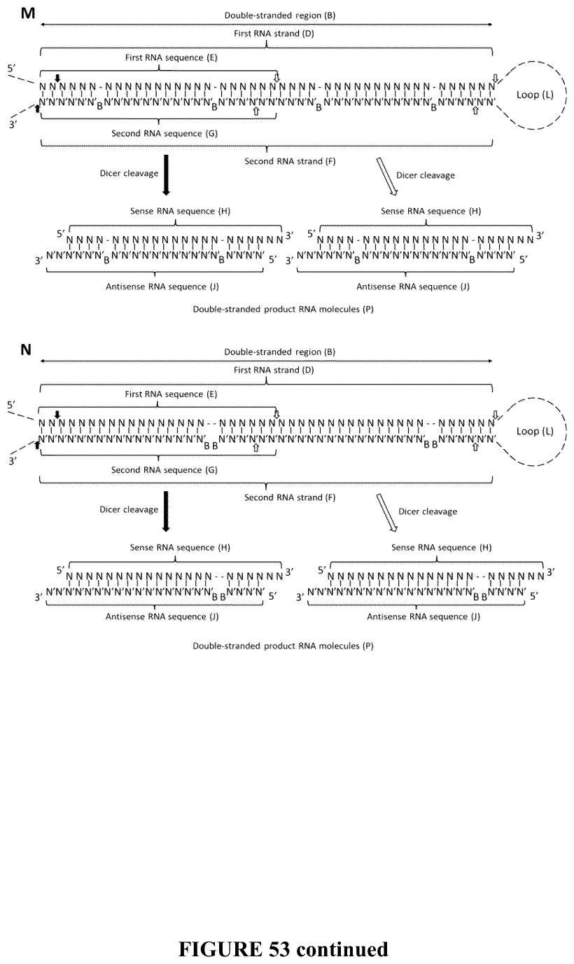

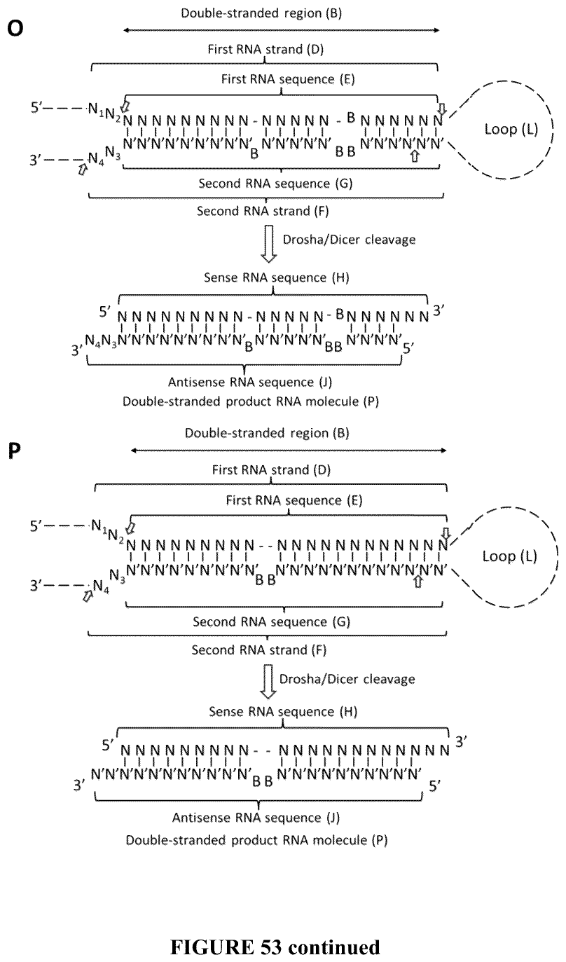

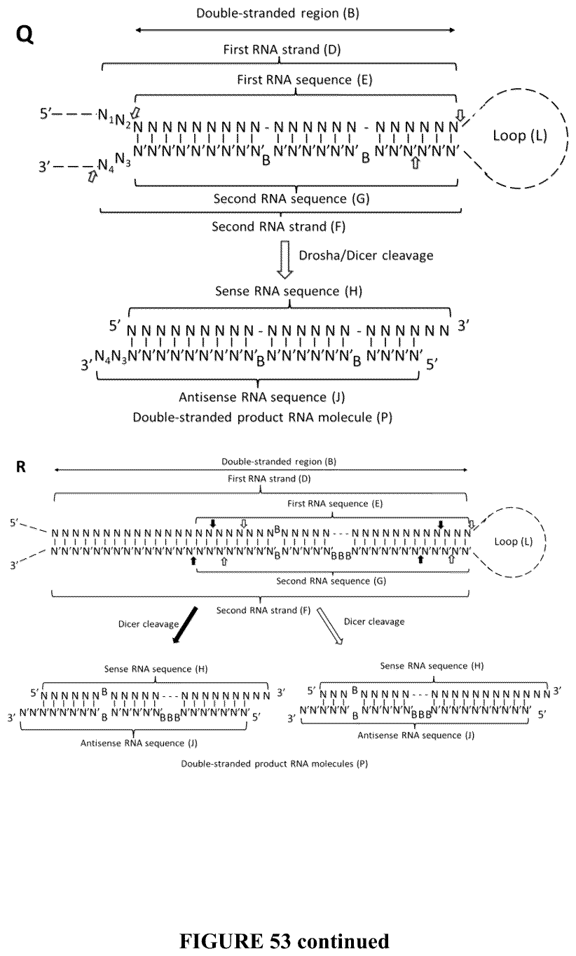

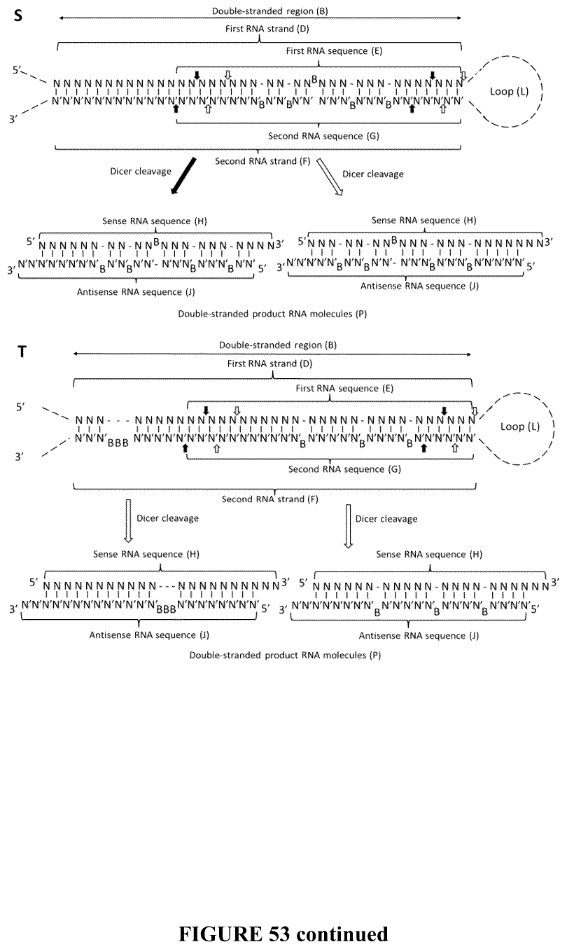

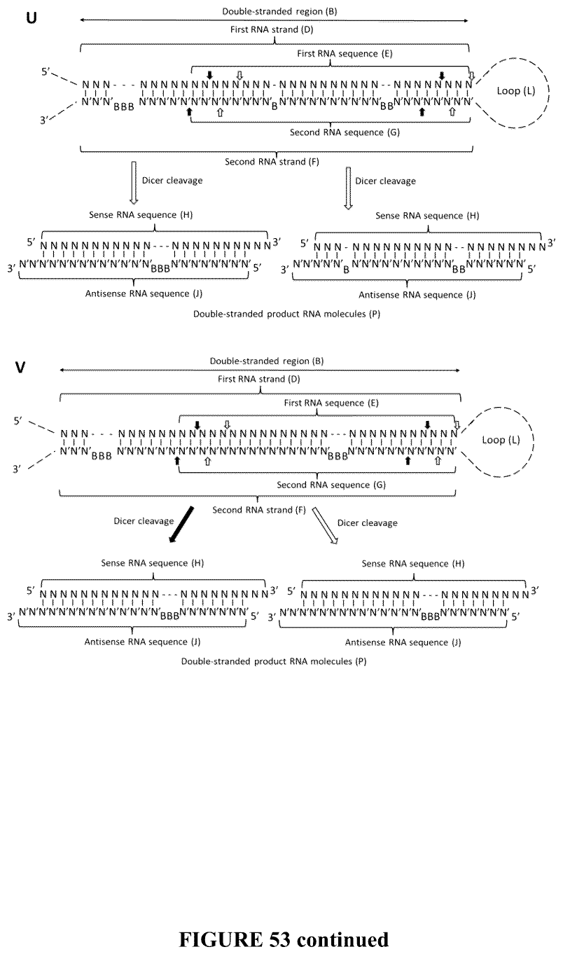

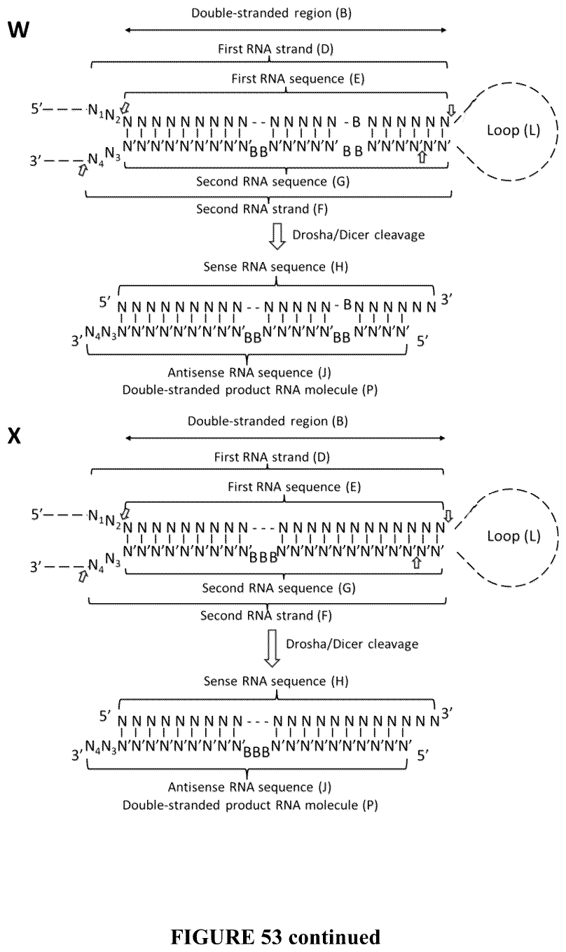

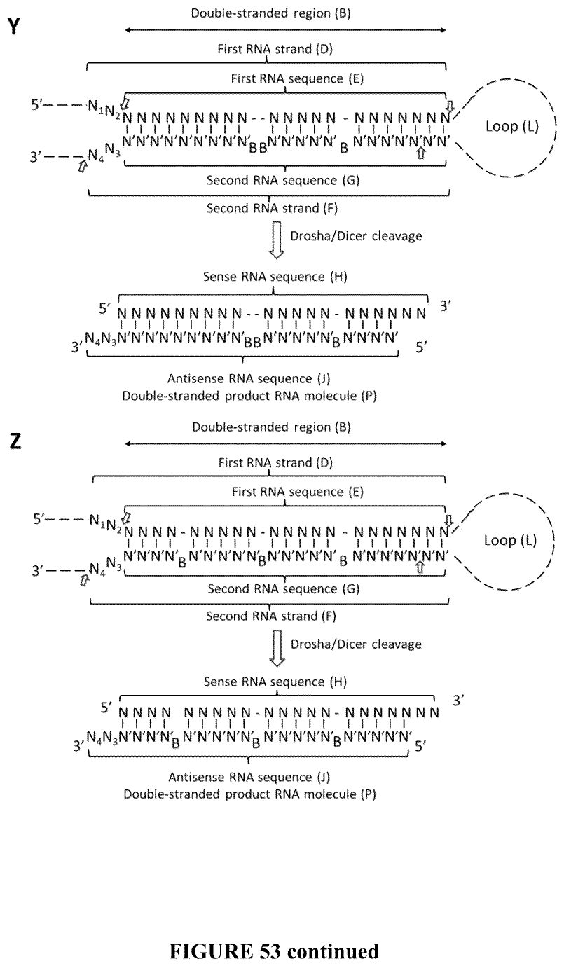

As used herein, a “bulge” refers to one or more contiguous ribonucleotides of the dsRNA region of the asymmetric RNA molecule which are not basepaired with another ribonucleotide, where the ribonucleotide or ribonucleotides of the bulge are immediately flanked by ribonucleotides which are basepaired in the dsRNA region. In a preferred embodiment, the antisense sequence of the dsRNA region of the asymmetric RNA molecules of the invention has more non-basepaired ribonucleotides than the sense sequence when the two are hybridised. In a more preferred embodiment, the sense strand of the dsRNA region lacks non-basepaired ribonucleotides i.e. all of the non-basepaired ribonucleotides in the dsRNA region are in the antisense sequence. These features are applied to both the precursor RNA molecules (A) of the invention and the product RNA molecules (P) of the invention, with reference to FIG. 53. In an embodiment, the antisense sequence of a dsRNA region of the asymmetric RNA molecule has 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 non-basepaired ribonucleotides, or at least any of those numbers, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 more non-basepaired ribonucleotides than the corresponding sense sequence of the dsRNA region. In an embodiment, the sense sequence of the dsRNA region has 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 non-basepaired ribonucleotides, preferably zero, but always less in number than the corresponding antisense sequence. All combinations of the numbers of non-basepaired ribonucleotides in the sense and antisense sequences are contemplated. In an embodiment, the antisense sequence is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ribonucleotides longer than the sense sequence to which it is hybridised. Again in this context, the number of non-basepaired ribonucleotides in the sense and antisense sequences are determined along the full length of the dsRNA region. These numbers can also be applied, independently, to a second or third dsRNA region in the precursor asymmetric RNA molecule.

Examples of a bulge of an RNA molecule of the invention include;
i) a single unpaired ribonucleotide such as shown in panels D, E and G, amongst others, of FIG. 53, where the antisense RNA sequence has at least one such bulge,
ii) two contiguous unpaired ribonucleotides such as shown in panels I and N, amongst others, of FIG. 53, where the antisense RNA sequence has at least one such bulge,
iii) three contiguous unpaired ribonucleotides such as shown in panels R and V, amongst others, of FIG. 53, where the antisense RNA sequence has at least one such bulge,
iv) a mismatched ribonucleotide pair, namely one ribonucleotide in the sense RNA sequence is aligned with, but not base-paired to, a ribonucleotide in the antisense RNA sequence, such as shown in panels A and I, amongst others, of FIG. 53,
v) two, three or four contiguous non-basepaired ribonucleotides in the antisense RNA sequence aligned with, but not basepaired to, a ribonucleotide in the sense RNA sequence, such as shown in panels B, O and W of FIG. 53. Combinations of bulges are also included:
vi) a combination of i) and iv) such as, for example, shown in panels A and F of FIG. 53,
vii) a combination of ii) and iv), such as, for example, shown in panel I of FIG. 53,
viii) a combination of iii) and iv) such as, for example, shown in panel R of FIG. 53,
ix) a combination of i) and ii) such as, for example, shown in panel U of FIG. 53, and
x) a combination of i) and iii), or i), ii) and iii), such as, for example, shown in panel U of FIG. 53.

In an embodiment, the precursor asymmetric RNA molecules of the invention comprise more bulged ribonucleotides in the antisense sequence of the dsRNA region than in the sense sequence of the dsRNA region. This feature is also true for the siRNAs of the invention produced by Dicer-mediated cleavage of the precursor RNA molecules, along the length of basepairing between the sense and antisense sequences in each siRNA. In an embodiment, the antisense sequences of siRNAs produced from the asymmetric RNA molecule have 1, 2, 3, 4, 5, or 6 non-basepaired ribonucleotides, or at least any of those numbers. Since longer dsRNA regions of the precursor RNA molecules will yield a population of different siRNA molecules, the population may include siRNAs with any combinations of those numbers, or within a range such as 1-6 ribonucleotides. In an embodiment, some of the antisense sequences of siRNAs produced from the asymmetric RNA molecule have 0 non-basepaired ribonucleotides and others have 1, 2, 3, 4, 5, or 6 non-basepaired ribonucleotides. In an embodiment, the sense sequences of siRNAs produced from the asymmetric RNA molecule have 0, 1, 2, 3, 4, 5, or 6 non-basepaired ribonucleotides, or at least any of those numbers. In a preferred embodiment, most or all of the sense sequences of siRNAs produced from the asymmetric RNA molecule have 0 non-basepaired ribonucleotides. In a more preferred embodiment, most or all of the sense sequences of siRNAs produced from the asymmetric RNA molecule have 0 non-basepaired ribonucleotides and the antisense sequences of siRNAs produced from the asymmetric RNA molecule have 1, 2, 3, 4, 5, or 6 non-basepaired ribonucleotides, or at least any of those numbers. For at least some of the asymmetric siRNAs, preferably most of them, for example for at least 60%, at least 70%, at least 80% or at least 90% of them, the number of non-basepaired ribonucleotides in the antisense sequence of the siRNA is 1, 2 or 3 more than the number in the sense sequence, for the Δ22, 423 or 424 modification, respectively. Clearly, a population of siRNAs produced from a precursor asymmetric RNA molecule may include both asymmetric and symmetric siRNAs, such as 21/21-mers, aside from any secondary siRNAs. For example, such a population may be produced from a chimeric dsRNA region where part has a 422, 423 or A24 modification and part does not have the modification.

In a preferred embodiment, the precursor, asymmetric RNA molecules with the Δ22 modification that are modified along the length of the dsRNA region have, on average, one more bulged ribonucleotide per 22 contiguous ribonucleotides in the antisense sequence of the dsRNA region than in the sense sequence. That is, in this embodiment, most if not all subsequences of 22 contiguous ribonucleotides from the antisense sequence have one more bulged ribonucleotide than the subsequences of 21 contiguous ribonucleotides from the sense sequence. Consequently, most if not all of the siRNAs produced from the dsRNA region will be 21/22-mers, although some 20/21-mers and 21/21-mers may also be produced. Such precursor asymmetric RNA molecules can be readily designed and made by deleting every 22^{nd} ribonucleotide from the sense sequence of the dsRNA region, relative to a corresponding symmetric RNA molecule which does not have bulged ribonucleotides. For example, the antisense subsequences, and the siRNAs produced, have 1, 2, 3 or 4 bulged ribonucleotides whereas the sense sequences have 0, 1, 2 or 3, respectively, bulged ribonucleotides.

The bulged ribonucleotides of the precursor asymmetric RNA molecules and the siRNAs produced by Dicer-mediated cleavage of the precursor RNA molecules are preferably from types i), ii), iii), ix) and x) above. Multiple bulges in any one of these types are also contemplated.

Various embodiments of the invention refer to a specific position or number of a ribonucleotide of a sense RNA sequence or an antisense RNA sequence. Such numbering is provided in relation to 5′ end of the sequence, as is standard in the art. For example, ribonucleotide number 1 is the ribonucleotide at the 5′ end of the sequence.

In one aspect, an asymmetric dsRNA molecule of the invention has a sense ribonucleotide sequence which is 21 ribonucleotides (21 nt) in length, hybridised to an antisense ribonucleotide sequence which is 22 ribonucleotides (22 nt) in length, where the molecule is produced by a Dicer from a precursor RNA molecule of the invention. A double-stranded RNA molecule that is the produced by Dicer is referred to herein as a “siRNA” and the sense and antisense ribonucleotide sequences in the siRNA, or separately, are referred to herein as “short RNAs” (sRNA). siRNAs include other products of Dicer, including 21/21-mers, 22/21-mers, 21/23-mers and 21/24-mers etc. The siRNAs of the invention have a typical structure that includes two, non-basepaired ribonucleotides at the 3′ ends of both the sense and antisense sequences, also termed “3′ overhangs”. Combinations of multiple 21/22-mers are included in the invention, where the individual siRNAs have only overlapping 22-mer antisense sequences derived from dsRNA regions of less than 44 basepairs in length, or include non-overlapping 22-mer antisense sequences from dsRNA regions of at least 44 basepairs in length, or both overlapping and non-overlapping antisense sequences. The hybridising sense sequences will also be overlapping or non-overlapping. The combinations of 21/22-mers may also comprise 21/21-mers or other siRNA lengths.

The class of 22 nt antisense sRNAs in plants have a specific functional property, namely these sRNAs can initiate the production of secondary siRNAs which are involved in transitive and systemic gene silencing (Cuperus et al., 2010; Taochy et al., 2017). These 22 nt sRNAs and the resulting secondary siRNAs represent a key aspect of plant defence against viruses (Qin et al., 2017; Wang et al., 2018), where DCL2-processed 22 nt siRNAs promote the production of 21 nt secondary siRNAs from the viral transcript via DCL4 (Bouche et al., 2006; Mlotshwa et al., 2008; Sanan-Mishra et al., 2021). Similarly, several 22 nt miRNAs found in plants have been shown to initiate secondary siRNA production to amplify gene silencing (Fujimoto and Iwakawa 2023). For example, the Arabidopsis thaliana 22 nt miRNAs, miR173 and miR828, target the transcripts of trans-acting loci TAS1, TAS2 and TAS4 for initial cleavage and then secondary siRNA production, where the secondary siRNAs silence PPR transcripts in the case of TAS1 and TAS2, and MYB transcription factors in the case of TAS4. This silencing is termed trans-acting silencing (TAS) (Allen et al., 2005; Hsieh et al., 2009; Chen et al., 2010; Luo et al., 2012). Such endogenous secondary siRNAs play an important role in a diverse range of processes including reproduction of monocotyledonous plants (Fan et al., 2016; Teng et al., 2020), regulation of defence response genes (Lopez-Marquez et al., 2021), and even cross-kingdom interactions between the parasitic vine, Cuscuta campestris, and its host (Shahid et al., 2018). RISCs loaded with 22 nt miRNAs initiate the production of phased secondary siRNAs (phasiRNAs) from the original cleaved target (Cuperus et al., 2010; Manavella et al., 2012; McHale et al., 2013; Liu et al., 2021). Upon initial cleavage of the target gene transcript, the 22 nt miRNA-AGO1 complex recruits the proteins Suppressor of Gene Silencing 3 (SGS3) and RNA dependent RNA polymerase 6 (RDR6) to 3′ fragment of the cleaved transcript. The RDR6 then synthesises the complementary RNA strand by RNA dependent RNA polymerase (RdRp) activity, thereby producing long dsRNA corresponding to the target RNA. The dsRNA molecules are then processed by DCL4 to produce 21 nt secondary siRNAs (Sakurai et al., 2021) which in turn associate with AGO to reduce the activity of the target transcript. Without being limited to this mechanism, the 22 nt antisense RNA molecules produced from the RNA molecules of the invention are thought to act through the same mechanism in at least plant cells, fungal cells and nematode cells.

As used herein, a “DCL4 protein” is a Dicer-like protein in plants which cleaves dsRNA substrates to predominantly produce 21/21-mer siRNAs. DCL4 is distinct from DCL1, DCL2 and DCL3 proteins. These four classes of DCL proteins are readily distinguishable by those of skill in the art, by sequence alignments with known members of each class or functional analysis. In Arabidopsis, DCL4 is involved in the production of 21 nt tasiRNAs through the sequential processing of long dsRNA precursors generated by the action of RDR6 on single-stranded RNA (Allen et al., 2005; Xie et al., 2005), 21-nt siRNAs from inverted repeat (IR)-derived or viral-derived dsRNAs (Henderson et al., 2006; Fusaro et al., 2006) and some miRNAs. DCL1, DCL2, DCL3 and DCL4 have specific roles in the biogenesis of distinct classes of endogenous sRNAs: 21 nt and 22 nt miRNAs are predominantly generated by DCL1, 22 nt siRNAs are produced mainly by DCL2, 24 nt repeat-associated siRNAs from transposons and retro-elements loci, repetitive DNA and reproductive phased siRNAs (phasiRNAs) are produced by DCL3 (Nagano et al., 2014) and another Dicer termed DCL5 in monocots, and 21-nt endogenous trans-acting (ta) siRNAs require the activity of DCL4. Functional DCL proteins contain several functional domains that typically consist of a DExD/H-box helicase domain, a DUF283 domain that acts as a RNA-binding domain, a PAZ domain, two RNaseIII domains and one or two dsRNA-binding domains (dsRBDs) (Montavon et al., 2018). Numerous DCL4 sequences are known from plant genomes: for example DCL4 in Arabidopsis thaliana (Accession no. DQ118423), Camelina sativa (XM_010456035.2), Brassica rapa (XM_009122569), Brassica napus (XM_048767418.1), etc. Homologs of DCL4 are known in other organisms. As used herein, a “DCL4 gene” encodes a DCL4 protein.

In another aspect, a dsRNA molecule (siRNA) of the invention has a sense ribonucleotide sequence which is 21 ribonucleotides in length, hybridised to an antisense ribonucleotide sequence which is 23 ribonucleotides (23 nt) in length, where the molecule is produced by a Dicer from a precursor RNA molecule of the invention. Combinations of multiple 21/23-mers are included in the invention, where the individual siRNAs have only overlapping 23-mer antisense sequences derived from dsRNA regions of less than 46 basepairs in length, or include non-overlapping 23-mer antisense sequences from dsRNA regions of at least 46 basepairs in length, or both overlapping and non-overlapping antisense sequences. The combinations of 21/23-mers may also comprise 21/21-mers, 21/22-mers or other siRNA lengths.

In a further aspect, a dsRNA molecule of the invention (siRNA) has a sense ribonucleotide sequence which is 21 ribonucleotides in length, hybridised to an antisense ribonucleotide sequence which is 24 ribonucleotides in length, where the molecule is produced by a Dicer from a precursor RNA molecule of the invention. Combinations of multiple 21/24-mers are included in the invention, where the individual siRNAs have only overlapping 24-mer antisense sequences derived from dsRNA regions of less than 48 basepairs in length, or include non-overlapping 24-mer antisense sequences from dsRNA regions of at least 48 basepairs in length, or both overlapping and non-overlapping antisense sequences. The combinations of 21/24-mers may also comprise 21/21-mers, 21/22-mers, 21/23-mers or other siRNA lengths.

In each case, the 22 nt, 23 nt or 24 nt antisense ribonucleotide sequence may comprise a 3′ ribonucleotide having a 2′-O-methyl group, added by a HEN1 protein after Dicer cleavage, and/or have a ribonucleotide at its 5′ end with more than one phosphate group for example a 5′ tri-phosphate group, or 5′ ribonucleotide may lack a 5′ phosphate group. Such modifications are considered to be in the scope of “a ribonucleotide” as used herein.

As used herein in the context of asymmetric RNA molecules, the term “precursor RNA molecule” or “RNA precursor” or similar phrase refers to an RNA molecule that is capable of being processed in a eukaryotic cell or in vitro to produce at least some antisense sRNA molecules of 22, 23 or 24 ribonucleotides in length, for example through enzymatic cleavage by RNases as described herein, regardless of whether any further processing occurs after the RNase cleavage. The skilled person will be aware of RNA precursors in the art, for example, RNA precursors which are subjected to structure-specific RNase cleavage, and will be aware that defined, multi-step pathways involving different cell compartments and molecular mechanisms are often involved—this may also apply to the enzymatic cleavage of the precursor RNA molecules of the invention.

In an embodiment, the precursor RNA molecule comprises two or more sense ribonucleotide sequences, and antisense ribonucleotide sequences hybridised thereto, which are identical in sequence to a region of a target RNA molecule. For example, the precursor RNA molecule can comprise at least 2, at least 3, at least 4, at least 5, at least 6, at least 7, at least 8, at least 9, at least 10, at least 11, at least 12 or more sense ribonucleotide sequences, and antisense ribonucleotide sequences hybridised thereto, which are identical in sequence to a region of a target RNA molecule. The sense ribonucleotide sequences may be identical to each other i.e. copies of a sequence, or variants but still targeting the same region of a target RNA molecule. Such a precursor RNA molecule provides an increased copy number of product siRNAs targeting the region of the target RNA molecule, intended to increase the efficiency of target gene down-regulation. Such precursor RNA molecules are also expected to be useful in reducing the activity of a family of related target RNA molecules. In this embodiment, any one or more or all of the sense/antisense sequences can be separated by a linking ribonucleotide sequence(s), for example a loop sequence.

In an embodiment, the two or more sense ribonucleotide sequences are different in that they are identical in sequence to different regions of the same target RNA molecule. The different regions of the target RNA molecule may be contiguous or non-contiguous. For example, the sequences can be identical to at least 2, at least 3, at least 4, at least 5, at least 6 or more regions of the one target RNA molecule. Again, those regions may be overlapping or non-overlapping, for example adjacent. In another example, two or more sense ribonucleotide sequences in the precursor RNA molecule are identical in sequence and at least one other sense ribonucleotide sequence is different in sequence.

In another embodiment, the two or more sense ribonucleotide sequences in the precursor RNA molecule are identical in sequence to regions of different target RNA molecules. For example, the sequences can be identical to at least 2, at least 3, at least 4, at least 5, at least 6 regions of different target RNA molecules i.e. the precursor RNA molecule is targeting multiple RNA molecules. In an embodiment, the multiple RNA target molecules are members of a gene family in that they are related in sequence. Alternatively, the multiple target RNA molecules are not related in sequence, for example one target RNA molecule is an endogenous plant RNA and the other is a viral RNA, insect RNA, fungal RNA or nematode RNA. Multiple different target RNAs from a virus, insect, fungus or nematode, or multiple viruses, insects, fungi or nematodes are also contemplated. The two or more sense RNA sequences with complementary antisense sequences may be joined together to form a longer dsRNA region in the precursor RNA molecule. See for example the chimeric hairpin RNA molecule described in Example 19 herein.

In an embodiment, the two or more sense ribonucleotide sequences have no intervening ribonucleotide sequences i.e. are directly covalently linked. In another embodiment, the two or more antisense ribonucleotide sequences have no intervening ribonucleotide sequences, or this applies to both the sense and antisense sequences. Alternatively, the two or more antisense ribonucleotide sequences are separated by an intervening ribonucleotide sequence and the two or more sense ribonucleotide sequences are directly covalently linked, or the converse.

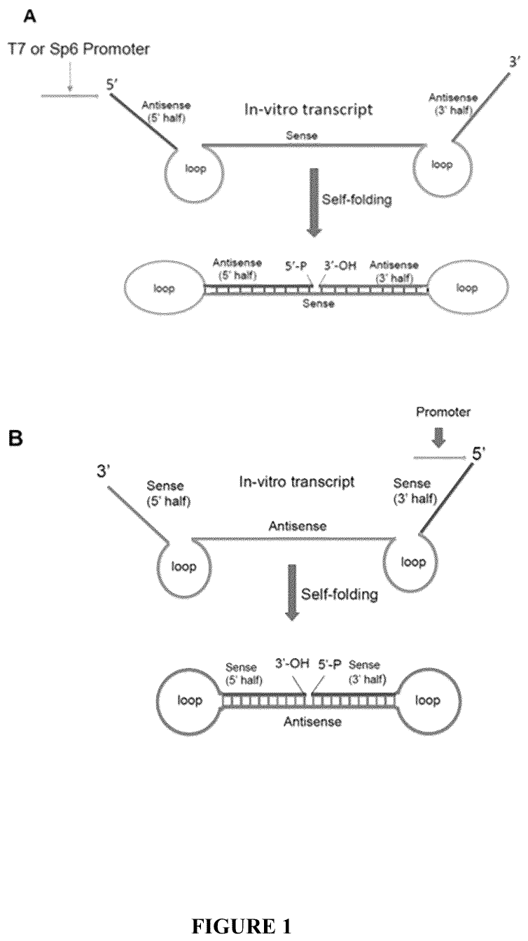

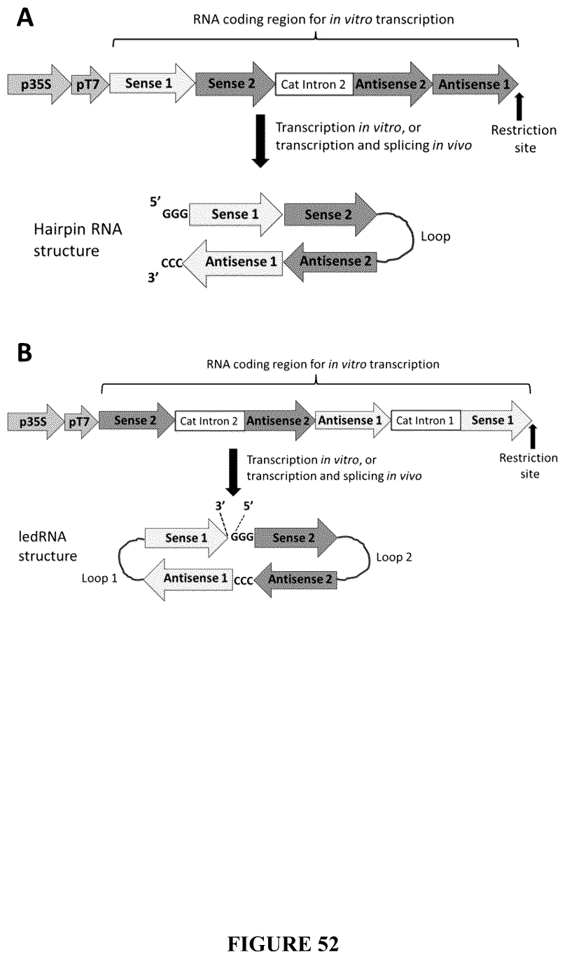

In an embodiment, the precursor asymmetric RNA molecule has a single strand of ribonucleotides having a 5′ end, at least one sense ribonucleotide sequence which is at least 21 ribonucleotides in length, an antisense ribonucleotide sequence which is at least 22 ribonucleotides in length hybridised to the sense ribonucleotide sequence over at least 20 of the at least 21 ribonucleotides to form a dsRNA region, where the sense ribonucleotide sequence and the antisense ribonucleotide sequence are covalently linked through an intervening linking ribonucleotide sequence, a second sense ribonucleotide sequence and a second antisense ribonucleotide sequence hybridised thereto which form a second dsRNA region, where the second sense sequence and the second antisense sequence are also covalently linked through an intervening linking ribonucleotide sequence, and a 3′ end. Such RNA molecules are referred to herein as “ledRNA” molecules or similar, where “led” is shorthand for “loop-ended”. Dicer cleavage of the first double-stranded region produces at least some 21/22-mer siRNAs of the invention. In a preferred embodiment, the ribonucleotide at the 5′ end of the first sense/antisense ribonucleotide sequences of the ledRNA and the ribonucleotide at 3′ end of the second sense/antisense sequences are not directly covalently bonded to each other but are rather positioned adjacent, with each basepaired to other ribonucleotides in the molecule. For example, see FIG. 1 parts A or B and FIG. 52, panel B.

In a preferred embodiment of the precursor asymmetric ledRNA molecule, the second sense ribonucleotide sequence is at least 21 ribonucleotides in length and the second antisense ribonucleotide sequence is at least 22 ribonucleotides in length and hybridised to the second sense ribonucleotide sequence through at least 20 basepairs. The dsRNA region formed by hybridisation between the second sense ribonucleotide and the second antisense ribonucleotide sequences may be longer, for example at least 44, at least 50, at least 60, at least 65, at least 70, at least 80, at least 84, at least 90, at least 100 basepairs in length. In this embodiment, the ledRNA molecule is essentially two asymmetric hairpin RNA molecules covalently joined together. The two asymmetric hairpins may target the same target RNA molecule or different target RNA molecules. In an alternative embodiment, the ledRNA molecule essentially comprises an asymmetric hairpin RNA molecule covalently linked to a symmetric hairpin RNA molecule.

In preferred forms of each of the ledRNA embodiments described above, one or preferably both of the antisense ribonucleotide sequences comprise non-basepaired ribonucleotides that form bulges, as described herein. The invention also provides the siRNA molecules produced by Dicer cleavage of the precursor ledRNA molecules, methods of their production and methods of their use.

In analogous embodiments, the invention provides precursor asymmetric ledRNA molecules which comprise sense RNA sequences of at least 21 ribonucleotides length and antisense RNA sequences which are at least 23 ribonucleotides in length, for the A23 modification, or at least 24 ribonucleotides in length for the A24 modification. The invention also provides the siRNA molecules produced by Dicer cleavage of these precursor ledRNA molecules, methods of their production and methods of their use.

In another embodiment, consecutive basepairs of RNA components are interspaced by at least one gap. In an embodiment, the “gap” is provided by an unpaired ribonucleotide in the hybridising sequence. In another embodiment, the “gap” is provided by the presence of an un-ligated 5′ leader sequence and/or 3′ trailer sequence. In this embodiment, the gap can be referred to as an “unligated gap”. Mismatches and unligated gap(s) can be located at various position(s) of the ledRNA molecule. For example, an unligated gap can immediately follow an antisense sequence. In another example, an unligated gap can be close to a linking RNA sequence (loop) of the ledRNA molecule. In another example, an unligated gap is positioned about equidistant between two loops. For example, see FIG. 1 parts A or B and FIG. 52, panel B.

In an embodiment, the precursor RNA molecule is a ledRNA molecule that consists of a single strand of RNA that folds back on itself. In an embodiment, the single strand is not circularly closed, for example, comprising an unligated gap. In another embodiment, the ledRNA molecule is a circularly closed molecule. Closed molecules can be produced by ligating an above referenced RNA molecule comprising an unligated gap, for example with an RNA ligase.

In an embodiment, the precursor RNA molecule of the invention comprises a 5′ or 3′ extension sequence, or both extension sequences. This is particularly the case for precursor RNA molecules produced by transcription in a cell. For example, the precursor RNA molecule can comprise a 5′ extension sequence which is covalently linked to the 5′ ribonucleotide of the first sense/antisense sequences. In another example, the RNA molecule comprises a 3′ extension sequence which is covalently linked to 3′ ribonucleotide of the second sense/antisense sequences. In another example, the RNA molecule can comprise a 5′ extension sequence which is covalently linked to the 5′ ribonucleotide of the first sense/antisense sequences and a 3′ extension sequence which is covalently linked to 3′ ribonucleotide of the second sense/antisense sequences. Further description of these sequences is provided below.

Each of the embodiments and the features described above for asymmetric RNA molecules can be applied to hairpin RNA molecules having a single-stranded loop sequence, or double-stranded RNA molecules formed by annealing between two RNA strands i.e. without a joining loop, or ledRNA molecules, described further as follows.

### LedRNA Molecule

In certain embodiments, precursor RNA molecules of the present invention comprise a first RNA component which is covalently linked to a second RNA component. In these embodiments, the precursor RNA molecule self-hybridizes or folds to form a “dumbbell” or “double hairpin” structure, referred to herein as a “ledRNA structure” or “ledRNA molecule”, for example see FIGS. 1 and 52B herein. As used herein in the context of ledRNA molecules, the term “precursor RNA molecule” or “RNA precursor” or similar phrase refers to an ledRNA molecule that is capable of being processed in a eukaryotic cell or in vitro to produce antisense sRNA molecules of 21, 22, 23 or 24 ribonucleotides in length, for example through enzymatic cleavage by RNases as described herein. In an embodiment, the precursor RNA molecule further comprises one or more of the following:
a linking ribonucleotide sequence which covalently links the first and second RNA components, i.e. the first and second RNA components are separated by an intervening RNA sequence which links them as a single RNA strand;
a 5′ leader sequence; and,
a 3′ trailer sequence.

In an embodiment of the ledRNA structure, the first RNA component consists of, in 5′ to 3′ order, a first 5′ ribonucleotide, a first RNA sequence and a first 3′ ribonucleotide, wherein the first 5′ and 3′ ribonucleotides basepair to each other in the RNA molecule, i.e. forming a terminal basepair of a first double-stranded region, wherein the first RNA sequence of the ledRNA molecule comprises a first sense RNA sequence of the invention, a first loop sequence of at least 4 ribonucleotides and a first antisense RNA sequence of the invention, wherein the first antisense RNA sequence hybridises with the first sense RNA sequence in the RNA molecule, i.e. forming at least part of the first double-stranded region, wherein the first antisense RNA sequence is capable of hybridising to a first region of a target RNA molecule. The first RNA sequence comprises, in 5′ to 3′ order, either the first sense sequence, the first loop sequence and then the first antisense sequence, or conversely the first antisense sequence, the loop sequence and then the first sense sequence, i.e. both arrangements are contemplated. In embodiments, the length of the first sense RNA sequence and the first antisense RNA sequence are both 31-50 ribonucleotides, or at least 44, at least 50, at least 60, at least 65, at least 70, at least 80, at least 86, at least 90 or at least 100 ribonucleotides. The length of both sequences may also be at least 150, at least 200, at least 250 or at least 300 ribonucleotides, to a maximum of the full length of the target RNA transcript. Preferred lengths are in the range 100-600 ribonucleotides. In an embodiment, the lengths of the first sense RNA sequence and the first antisense RNA sequence are equal. In a preferred embodiment, for an asymmetric first RNA component, the first antisense sequence is longer than the first sense sequence by virtue of the presence of non-basepaired, bulged ribonucleotides in the first antisense sequence. If all of the ribonucleotides of the first sense and antisense sequences in the dsRNA region are basepaired, the length of the dsRNA region in basepairs is equal to length of the ribonucleotides, whereas if not all of the ribonucleotides of the sense and antisense sequences are basepaired, for example where the antisense sequence comprises bulged ribonucleotides, the length of the dsRNA region in basepairs is less than the number of ribonucleotides in one or other sequence. In an embodiment, the number of basepairs is equal to the number of ribonucleotides in the sense sequence and less than the number of ribonucleotides in the antisense sequence, where the antisense sequence has bulged ribonucleotides. In preferred embodiments, the length of the sense sequence is between 87-97% or 87-96% of the length of the antisense sequence, preferably 91-97% or 91-96%, or more preferably 94-97% or 94-96% of the length of the antisense sequence.

In the ledRNA structure, the second RNA component consists of, in 5′ to 3′ order, a second 5′ ribonucleotide, a second RNA sequence and a second 3′ ribonucleotide, wherein the second 5′ and 3′ ribonucleotides basepair, i.e. forming a terminal basepair of the second double-stranded region, wherein the second RNA sequence comprises a second sense RNA sequence, a second loop sequence of at least 4 ribonucleotides and a second antisense RNA sequence, wherein the second sense RNA sequence hybridises with the second antisense RNA sequence, i.e. forming at least part of the second double-stranded region. In this embodiment, the basepair formed between the second 5′ ribonucleotide and the second 3′ ribonucleotide is considered to be a terminal basepair of the dsRNA region formed by self-hybridisation of the second RNA component. Analogous but independently to the first RNA sequence, the second RNA sequence comprises, in 5′ to 3′ order, either the second sense sequence, the second loop sequence and then the second antisense sequence, or conversely the second antisense sequence, the second loop sequence and then the second sense sequence, again both arrangements are contemplated. The length and basepairing features described above for the dsRNA region of the first RNA component equally apply to the dsRNA region of the second RNA component.

In these embodiments, the first RNA component and the second RNA component both form stem-loop or hairpin RNA structures, and the ledRNA essentially comprises at least two, preferably only two, hairpin RNAs joined together that form the “dumbbell structure” (see for example FIGS. 1 and 52B).

In an embodiment, the precursor RNA molecules of the present invention, including the ledRNA molecules, comprise two or more sense ribonucleotide sequences which are each capable of hybridising to regions of one (contiguous) antisense ribonucleotide sequence in the precursor RNA molecule to form an extended dsRNA region, for example see FIG. 1B. In an embodiment, the precursor RNA molecules of the present invention, including the ledRNA molecules, comprise two or more antisense sense ribonucleotide sequences which are each capable of hybridising to regions of one (contiguous) sense ribonucleotide sequence to form a dsRNA region, for example see FIG. 1A. In an embodiment, the precursor RNA molecules of the present invention, including the ledRNA molecules, comprise two or more antisense ribonucleotide sequences and two or more sense ribonucleotide sequences wherein each antisense ribonucleotide sequence is capable of hybridising to a sense ribonucleotide sequence to form two or more dsRNA regions. In an embodiment, one or preferably both dsRNA regions comprise some non-canonical basepairing, more preferably G:U basepairing. In an embodiment, one or preferably both dsRNA regions comprise some non-basepaired ribonucleotides in the antisense sequence(s), with or without G:U basepairing. All of the features described for the asymmetric RNA molecules are applicable here.

In another, preferred embodiment, the first sense RNA sequence is directly covalently linked to the first 5′ ribonucleotide without any intervening nucleotides, or the first antisense RNA sequence is directly covalently linked to the first 3′ ribonucleotide without any intervening ribonucleotides, or both, i.e. the first sense RNA sequence is directly covalently linked to the first 5′ ribonucleotide without any intervening ribonucleotides and the first antisense RNA sequence is directly covalently linked to the first 3′ ribonucleotide without any intervening ribonucleotides. In another embodiment, there are at least one, at least two, at least three, at least four, at least five, at least six, at least seven, at least eight, at least nine, at least 10 intervening ribonucleotides between the first 5′ ribonucleotide and the first sense RNA sequence and/or between the first 3′ ribonucleotide and the first antisense RNA sequence. It is understood that such intervening ribonucleotides may be unrelated in sequence to the target RNA molecule but may assist in stabilising the basepairing of adjacent sense and antisense sequences.

In an embodiment, the above referenced first and second RNA components are covalently linked via an intervening linking ribonucleotide sequence. In an embodiment, the linking ribonucleotide sequence acts as a spacer between the first sense RNA sequence and the second sense RNA sequence. Alternatively, the first sense RNA sequence and the second sense RNA sequence are covalently joined without an intervening spacer, thereby forming an extended sense sequence, for example see FIG. 1A. In that embodiment, the two sense sequences may be contiguous with regard to a region of the target RNA transcript, or they may target non-contiguous regions of a target RNA transcript, or different target RNA transcripts. In these embodiments, the first and second antisense RNA sequences flank the first and second sense RNA sequences. In an alternative embodiment, the linking ribonucleotide sequence acts as a spacer between the first antisense RNA sequence and the second antisense RNA sequence. Alternatively, the first antisense RNA sequence and the second antisense RNA sequence are covalently joined without an intervening spacer, thereby forming an extended antisense sequence, for example see FIG. 1B. In that embodiment, the two antisense sequences may be contiguous with regard to the complement of a region of the target RNA transcript, or to non-contiguous regions of the complement of a target RNA transcript, or different target RNA transcripts. In these embodiments, the first and second sense RNA sequences flank the first and second antisense RNA sequences, see FIGS. 1B and 52B.

The intervening spacer may also join the first sense RNA sequence and the second antisense RNA sequence i.e. connect the first 5′ ribonucleotide and the second 3′ ribonucleotide, or the second sense RNA sequence and the first antisense RNA sequence i.e connect the second 5′ ribonucleotide to the first 3′ ribonucleotide. The order of elements would thereby be alternating for the sense and antisense RNA sequences i.e sense-antisense-linker-sense-antisense, or antisense-sense-linker-antisense-sense in the 5′ to 3′ order.

In each RNA component of the double hairpin structure of the ledRNA molecule, the order of the sense and antisense RNA sequences in each hairpin, in 5′ to 3′ order, may independently be either sense then antisense, or antisense then sense. In preferred embodiments, the order of the sense and antisense sequences in the double hairpin structure of the RNA molecule is either antisense-sense-sense-antisense where the two sense sequences may be contiguous relative to the target RNA molecule, or sense-antisense-antisense-sense where the two antisense sequences are contiguous relative to the complement of a region of the target RNA transcript.

In an embodiment, the linking ribonucleotide sequence acts as a spacer between a first or second RNA component and a loop. In another embodiment, the RNA molecule comprises multiple sense ribonucleotide sequences, i.e. multiple copies, that are substantially identical in sequence to a first region of a target RNA molecule and a linking ribonucleotide sequence which acts as a spacer between these sequences. In an embodiment, at least two, at least three, at least four, at least five, at least six, at least seven, at least eight, at least nine, at least 10 ribonucleotide sequences that are substantially identical in sequence to a first region of a target RNA molecule are provided in the RNA molecule, each being separated from the other(s) by a linking ribonucleotide sequence.

In an embodiment, the RNA molecule has a modified 5′ or 3′ end, for example by attachment of a lipid group such as cholesterol, or a vitamin such as biotin, or a polypeptide such as a cell-penetrating peptide. Such modifications may assist in the uptake of the RNA molecule into the eukaryotic cell where the RNA is to function.

In an embodiment, the first RNA component, or both the first and second RNA components, comprises an antisense RNA sequence of at least 22 contiguous ribonucleotides, preferably at least 42, or at least 65, or at least 86 contiguous ribonucleotides, that is substantially identical, preferably fully identical, in sequence to the complement of a region of a target RNA molecule, and a sense RNA sequence that hybridises to the antisense RNA sequence. In an embodiment, each RNA component has an antisense RNA sequence that is substantially identical in sequence to the complement of a region of the same target RNA molecule i.e. the RNA components target the same target RNA sequence. In an alternative embodiment, the RNA components have antisense RNA sequences that are substantially identical in sequence to the complements of different regions of the same target RNA molecule. In an embodiment, the RNA components have antisense RNA sequences that are substantially identical in sequence to the complements of regions of different target RNA molecules i.e. the RNA molecule can be used to reduce the expression and/or activity of two or more target RNA molecules which may be unrelated in sequence. For example, the RNA molecule of the invention can comprise 2, 3, 4, 5, 6, 7, 8, 9, 10, 11, 12 or more antisense ribonucleotide sequences, and sense ribonucleotide sequences basepaired thereto, which antisense ribonucleotide sequences are each independently identical in sequence to the complement of a region of a target RNA molecule. In an embodiment, the antisense sequences can be identical to the complements of at least 2, at least 3, at least 4, at least 5, at least 6 regions of different target molecules. Clearly, combinations of antisense RNA sequences are contemplated, complementary to different target RNA sequences on the same or different target RNA molecules.

In an embodiment, the ledRNA molecule of the invention is fully basepaired in the dsRNA region of the first RNA component or fully basepaired in the dsRNA region of the second RNA component, preferably fully basepaired in both dsRNA regions. Some of the basepairs may be G:U basepairs, for example at least one, but less than 5%, of the basepairs in one or both of the dsRNA regions are G:U basepairs. In an embodiment, both dsRNA regions are fully basepaired and all of the basepairs in the dsRNA regions are canonical basepairs. In a preferred embodiment, both dsRNA regions are fully basepaired and 5-40% of the basepairs in at least one of the dsRNA regions, preferably both dsRNA regions, are basepaired in noncanonical basepairs such as G:U basepairs. In another embodiment, one or both dsRNA regions are not fully basepaired, for example having one or more bulging ribonucleotides in the antisense sequence(s), and 5-40% of the basepairs in at least one of the dsRNA regions, preferably both dsRNA regions, are basepaired in noncanonical basepairs such as G:U basepairs. In an embodiment, both dsRNA regions have one or more bulging antisense ribonucleotides i.e. is asymmetric, and one, but not the other, dsRNA region comprises 5-40% G:U basepairs. In a preferred embodiment, both dsRNA regions have one or more bulging antisense ribonucleotides and comprise 5-40% G:U basepairs. Preferably, on average one in each 22, or 23 or 24, ribonucleotides in the antisense sequence is a bulged ribonucleotide, along the full length of the dsRNA region(s). It is preferred for each of these embodiments of the ledRNA molecules that at least 50%, at least 60%, at least 70%, at least 80%, at least 90% or even 100% of the G:U basepairs in at least one dsRNA region, preferably both dsRNA regions independently, are formed by nucleotide substitutions which are A to G, or C to U, or a combination of A to G and C to U substitutions, in the sense sequence of at least one or both dsRNA regions of the led RNA molecules relative to a corresponding ledRNA molecule having only canonical basepairing. In each of these embodiments, the percentage of non-canonical basepairs such as G:U basepairs or the average number of bulged ribonucleotides is calculated over the full length of the dsRNA region.

The present invention thereby also provides for a combination of the ledRNA molecule structures with the asymmetric RNA design having one or more of the Δ22, Δ23 or Δ24 modifications, either with or without the additional presence of noncanonical basepairings such as G:U basepairing, particularly having longer dsRNA regions than 30 basepairs, allowing for the production of multiple, non-overlapping antisense 22-mers, 23-mers or 24-mers that hybridise to one or more target RNA transcripts. In an embodiment with the Δ22 modification, the first RNA component of the ledRNA molecule comprises a first RNA strand comprising a sense sequence of at least 44 ribonucleotides and a second RNA strand comprising an antisense sequence of at least 46 ribonucleotides, wherein 2, 3 or 4 ribonucleotides of the at least 46 contiguous ribonucleotides of the antisense sequence, and 0, 1 or 2, respectively, ribonucleotides of the at least 44 contiguous ribonucleotides of the sense sequence are non-basepaired in the dsRNA regions of the first RNA component, the non-basepaired ribonucleotides forming bulges in the dsRNA region(s). Preferably, only 2 or 3, more preferably only 2 ribonucleotides, of the 46 ribonucleotides of the antisense sequence are non-basepaired in at least one, or both, dsRNA regions. This feature may also be applied to the second RNA component.

The ledRNA molecule may have one or more even longer dsRNA regions which have asymmetric modification, preferably both dsRNA regions have the modification, preferably the Δ22 modification. For example, in an embodiment, the first RNA component comprises a sense sequence of at least 65 contiguous ribonucleotides and an antisense sequence which comprises at least 68 contiguous ribonucleotides, wherein 3, 4, 5 or 6 ribonucleotides of the at least 68 contiguous ribonucleotides of the antisense sequence, and 0, 1, 2 or 3, respectively, ribonucleotides of the at least 65 contiguous ribonucleotides of the sense sequence are non-basepaired in the dsRNA region of the first RNA component, the non-basepaired ribonucleotides forming bulges. In this embodiment, the sense RNA sequence hybridises to the antisense RNA sequence by basepairing between at least 62 of the at least 65 contiguous ribonucleotides of the sense sequence and at least 62 of the at least 68 contiguous ribonucleotides of the antisense RNA sequence, which are either all canonical basepairs or some canonical basepairs and 5-40% G:U basepairs. In a further, longer embodiment, the sense RNA sequence comprises at least 86 contiguous ribonucleotides and the antisense RNA sequence comprises at least 90 contiguous ribonucleotides, and 4, 5, 6, 7 or 8 ribonucleotides of the at least 90 contiguous ribonucleotides of the antisense sequence, and 0, 1, 2, 3 or 4, respectively, ribonucleotides of the at least 86 contiguous ribonucleotides of the sense sequence are non-basepaired in the dsRNA regions of the first RNA component, the non-basepaired ribonucleotides forming bulges. In the same manner, one or more dsRNA regions may have even longer dsRNA regions. In another embodiment, the Δ22, Δ23 or Δ24 modification is not applied to the full length of the dsRNA region i.e. the modification is applied to one subregion of the dsRNA region but not to another subregion of the dsRNA molecule. For example, the dsRNA region may have a length of at least 100 or at least 200 basepairs and comprise the lengths of sense and asymmetric antisense sequences mentioned above. All of these features may also be applied to the second RNA component.

Each of the above embodiments of the ledRNA molecules is best used to reduce the amount and/or activity of a target RNA transcript in a eukaryotic cell, preferably in a plant cell such as an endogenous plant RNA transcript or an RNA of a viral pathogen of a plant, or a fungal RNA transcript such as a plant-pathogenic fungus (PPF) or animal-pathogenic fungus, or an invertebrate animal RNA transcript such as a nematode transcript, particularly a plant-pathogenic nematode (PPN). This can be achieved either by expression of a genetic construct encoding the ledRNA molecule in planta, or produced in vitro or in a host cell such as a yeast cell and applied topically to the plant, PPF or PPN.

### Non-Canonical Basepairing

The present invention provides precursor RNA molecules, including the hairpin RNA molecules, ledRNA molecules and double-stranded RNA molecules having two separate RNA strands annealed together and lacking a loop sequence, which comprise one or more sense ribonucleotide sequence(s) and one or more antisense ribonucleotide sequence(s) which are capable of hybridising to each other to form one or more double-stranded (ds) RNA region(s) with some non-canonical basepairing i.e. with a combination of canonical and non-canonical basepairing. The present invention also provides product siRNA molecules (P) produced by processing of the precursor RNA molecules by Dicer, comprising a sense ribonucleotide sequence and an antisense ribonucleotide sequence that hybridise with some non-canonical basepairing.

In the following embodiments, the full length of the dsRNA region (i.e. the whole dsRNA region) of the precursor RNA molecule of the invention is considered as the context for the feature if there is only one dsRNA region, or for each of the dsRNA regions of the precursor RNA molecule if there are two or more dsRNA regions in the RNA molecule. In an embodiment, at least 5% of the basepairs in a dsRNA region are non-canonical basepairs, preferably all G:U basepairs. In a related but non-identical embodiment, at least 5% of the ribonucleotides, in total, in a dsRNA region are basepaired in non-canonical basepairs, preferably in G:U basepairs. In an embodiment, at least 6% of the basepairs in a dsRNA region are non-canonical basepairs, preferably all G:U basepairs. In an embodiment, at least 7% of the basepairs in a dsRNA region are non-canonical basepairs, preferably all G:U basepairs. In an embodiment, at least 8% of the basepairs in a dsRNA region are non-canonical basepairs, preferably all G:U basepairs. In an embodiment, at least 9% or 10% of the basepairs in a dsRNA region are non-canonical basepairs, preferably all G:U basepairs. In an embodiment, at least 11% or 12% of the basepairs in a dsRNA region are non-canonical basepairs, preferably all G:U basepairs. In an embodiment, at least 15%, or about 15%, of the basepairs in a dsRNA region are non-canonical basepairs, preferably all G:U basepairs. In an embodiment, at least 20%, or about 20%, of the basepairs in a dsRNA region are non-canonical basepairs, preferably all G:U basepairs. In an embodiment, at least 25%, or about 25%, of the basepairs in a dsRNA region are non-canonical basepairs. In an embodiment, at least 30%, or about 30%, of the basepairs in a dsRNA region are non-canonical basepairs, preferably all G:U basepairs. In each of these embodiments, it is preferred that a maximum of 40% of the basepairs in the dsRNA region are non-canonical basepairs, more preferably a maximum of 35% of the basepairs in the dsRNA region are non-canonical basepairs, still more preferably a maximum of 30% of the basepairs in the dsRNA region are non-canonical basepairs. In an embodiment, less preferred, about 35% of the basepairs in a dsRNA region are non-canonical basepairs. In an embodiment, even less preferred, about 40% of the basepairs in a dsRNA region are non-canonical basepairs. In related but non-identical embodiments, at least 6%, at least 7%, at least 8%, at least 9%, at least 10%, at least 11%, at least 12%, at least 15%, about 15%, at least 20%, about 20%, at least 25%, about 25%, at least 30%, about 30%, to a maximum of 40%, preferably 35%, even more preferably a maximum of 30%, of the ribonucleotides, in total, in a dsRNA region are basepaired in non-canonical basepairs, preferably in G:U basepairs. Each of these ranges can be applied independently to each of the dsRNA regions in a precursor molecule comprising multiple dsRNA regions.

In an embodiment, between 10% and 40% of the basepairs in a dsRNA region of the precursor RNA molecule of the invention, including the ledRNA molecules, and in the product RNA molecules (P), are non-canonical basepairs, preferably all G:U basepairs. In an embodiment, between 10% and 35% of the basepairs in a dsRNA region are non-canonical basepairs, preferably all G:U basepairs. In an embodiment, between 10% and 30% of the basepairs in a dsRNA region are non-canonical basepairs, preferably all G:U basepairs. In an embodiment, between 10% and 25% of the basepairs in a dsRNA region are non-canonical basepairs, preferably all G:U basepairs. In an embodiment, between 10% and 20% of the basepairs in a dsRNA region are non-canonical basepairs, preferably all G:U basepairs. In an embodiment, between 10% and 15% of the basepairs in a dsRNA region are non-canonical basepairs, preferably all G:U basepairs. In an embodiment, between 15% and 30% of the basepairs in a dsRNA region are non-canonical basepairs, preferably all G:U basepairs. In an embodiment, between 15% and 25% of the basepairs in a dsRNA region are non-canonical basepairs, preferably all G:U basepairs. In an embodiment, between 15% and 20% of the basepairs in a dsRNA region are non-canonical basepairs, preferably all G:U basepairs. In an embodiment, between 5% and 30% of the basepairs in a dsRNA region are non-canonical basepairs, preferably all G:U basepairs. In an embodiment, between 5% and 25% of the basepairs in a dsRNA region are non-canonical basepairs, preferably all G:U basepairs. In an embodiment, between 5% and 20% of the basepairs in a dsRNA region are non-canonical basepairs, preferably all G:U basepairs. In an embodiment, between 5% and 15% of the basepairs in a dsRNA region are non-canonical basepairs, preferably all G:U basepairs. In an embodiment, between 5% and 10% of the basepairs in a dsRNA region are non-canonical basepairs, preferably all G:U basepairs. In related but non-identical embodiments, between 10% and 40%, between 10% and 35%, between 10% and 30%, between 10% and 25%, between 10% and 20%, between 10% and 15%, between 15% and 30%, between 15% and 25%, between 15% and 20%, between 5% and 30%, between 5% and 25%, between 5% and 20%, between 5% and 15%, or between 5% and 10% of the ribonucleotides, in total, in a dsRNA region are basepaired in non-canonical basepairs, preferably all such ribonucleotides are in G:U basepairs.

In each of the above embodiments, it is preferred that the noncanonical basepairs are distributed along the dsRNA region in a regularly-spaced manner, for example on average one non-canonical basepair, preferably a G:U basepair, about every 4, 5, 6, 7, 8 or 9 basepairs. A preferred range is one non-canonical basepair, preferably a G:U basepair, on average every 4-8 or 4-7 basepairs or 4-6 basepairs. Again, this is preferably calculated along the full length of the dsRNA region. However, the average frequency of non-canonical basepairs such as G:U basepairs may vary across the full length of the dsRNA region, depending for example on the content of A and C bases in the sense sequence. Not all A nucleotides in the sense sequence need be substituted with G nucleotides, and/or not all C nucleotides need be substituted with U nucleotides in the precursor RNA molecule. In an embodiment, the precursor RNA molecule such as a hairpin RNA or ledRNA molecule, or a product RNA molecule (P), does not comprise a double-stranded region with greater than 11 contiguous canonical base-pairs, preferably lacks 10 or 11 contiguous canonical basepairs, more preferably lacks 9 contiguous canonical basepairs, even more preferably lacks 8 contiguous canonical basepairs. Alternatively, in embodiments, the longest stretch of contiguous canonical basepairing in the dsRNA region, or all dsRNA regions in the precursor RNA molecule, is 7, 8, 9, 10, 11, 12, 13, or 14 basepairs. Less preferred, the longest stretch has 15, 16, 17, 18 or 19 contiguous canonical basepairs.

In an embodiment, a precursor dsRNA molecule of the invention such as the hairpin RNA or ledRNA molecule comprises 20 contiguous basepairs, wherein at least one basepair, or only one basepair, of the 20 basepairs is a non-canonical basepair. In an embodiment, the dsRNA region comprises 20 basepairs, wherein at least 2 basepairs, or exactly 2 basepairs, of the 20 basepairs are non-canonical basepairs. In an embodiment, the dsRNA region comprises 20 basepairs, wherein at least 3 basepairs, or exactly 3 basepairs, of the 20 basepairs are non-canonical basepairs. In an embodiment, the dsRNA region comprises 20 basepairs, wherein at least 4 basepairs, or exactly 4 basepairs, of the 20 basepairs are non-canonical basepairs. In an embodiment, the dsRNA region comprises 20 basepairs, wherein at least 5 basepairs, or exactly 5 basepairs, of the 20 basepairs are non-canonical basepairs. In an embodiment, the dsRNA region comprises 20 basepairs, wherein at least 6 basepairs, or exactly 6 basepairs, of the 20 basepairs are non-canonical basepairs. In an embodiment, the dsRNA region comprises 20 basepairs, wherein at least 7 basepairs, or exactly 7 basepairs, of the 20 basepairs are non-canonical basepairs. In an embodiment, the dsRNA region comprises 20 basepairs, wherein at least 8 basepairs, or exactly 8 basepairs, of the 20 basepairs are non-canonical basepairs. In an embodiment, the dsRNA region comprises 20 basepairs, wherein at least 9 basepairs, or exactly 9 basepairs, of the 20 basepairs are non-canonical basepairs. In each of these embodiments, it is preferred that a maximum of 10 of the 20 basepairs in the dsRNA region are non-canonical basepairs, more preferably a maximum of 9 of the basepairs in the dsRNA region are non-canonical basepairs, still more preferably a maximum of 8 of the basepairs in the dsRNA region are non-canonical basepairs, even still more preferably a maximum of 7 of the basepairs in the dsRNA region are non-canonical basepairs, and most preferably a maximum of 6 of the basepairs in the dsRNA region are non-canonical basepairs. Preferably, in each of the above embodiments, the non-canonical basepairs comprise at least one G:U basepair, more preferably all of the non-canonical basepairs are G:U basepairs. Also preferred, more of the G:U basepairs have the G ribonucleotide in the sense sequence and the U ribonucleotide in the antisense sequence than the converse. In an embodiment, at least twice as many, or at least three times as many, of the G:U basepairs have the G ribonucleotide in the sense sequence, or more preferred all of the G:U basepairs have the G ribonucleotide in the sense sequence. In an embodiment, a non-basepaired ribonucleotide bulge is adjacent to a G:U basepair on one side, or to G:U basepairs on both sides. Alternatively, each bulge is adjacent to canonical basepairs on both sides.

In an embodiment, a dsRNA molecule of the invention, such as a “double-stranded product RNA molecule (P)” of the invention, comprises 19 basepairs, wherein at least one basepair, or only one basepair, of the 19 basepairs is a non-canonical basepair, preferably a G:U basepair where the G is in the sense RNA sequence (H) and the U is in the antisense RNA sequence (J), more preferably the G is ribonucleotide 19 of the sense RNA sequence (H) and the U is ribonucleotide 1 of the antisense RNA sequence (J). In an embodiment, the dsRNA region comprises 19 basepairs, wherein at least 2 basepairs, or exactly 2 basepairs, of the 19 basepairs are non-canonical basepairs. In an embodiment, the dsRNA region comprises 19 basepairs, wherein at least 3 basepairs, or exactly 3 basepairs, of the 19 basepairs are non-canonical basepairs. In an embodiment, the dsRNA region comprises 19 basepairs, wherein at least 4 basepairs, or exactly 4 basepairs, of the 19 basepairs are non-canonical basepairs. In an embodiment, the dsRNA region comprises 19 basepairs, wherein at least 5 basepairs, or exactly 5 basepairs, of the 19 basepairs are non-canonical basepairs. In an embodiment, the dsRNA region comprises 19 basepairs, wherein at least 6 basepairs, or exactly 6 basepairs, of the 19 basepairs are non-canonical basepairs. In an embodiment, the dsRNA region comprises 19 basepairs, wherein at least 7 basepairs, or exactly 7 basepairs, of the 19 basepairs are non-canonical basepairs. In an embodiment, the dsRNA region comprises 19 basepairs, wherein at least 8 basepairs, or exactly 8 basepairs, of the 19 basepairs are non-canonical basepairs. In an embodiment, the dsRNA region comprises 19 basepairs, wherein at least 9 basepairs, or exactly 9 basepairs, of the 19 basepairs are non-canonical basepairs. In each of these embodiments, it is preferred that a maximum of 10 of the 19 basepairs in the dsRNA region are non-canonical basepairs, more preferably a maximum of 9 of the basepairs in the dsRNA region are non-canonical basepairs, still more preferably a maximum of 8 of the basepairs in the dsRNA region are non-canonical basepairs, even still more preferably a maximum of 7 of the basepairs in the dsRNA region are non-canonical basepairs, and most preferably a maximum of 6 of the basepairs in the dsRNA region are non-canonical basepairs. Preferably, in the above embodiments, the non-canonical basepairs comprise at least one G:U basepair, more preferably all of the non-canonical basepairs are G:U basepairs. Also preferred, more of the G:U basepairs have the G ribonucleotide in the sense sequence (H) and the U ribonucleotide in the antisense sequence (J) than the converse. In an embodiment, at least twice as many, or at least three times as many, of the G:U basepairs have the G ribonucleotide in the sense sequence (H), or more preferred all of the G:U basepairs have the G ribonucleotide in the sense sequence (H). In each of the embodiments described in this paragraph, in the context of the Δ22, Δ23 and Δ24 modifications, the 19 basepairs are not contiguous by virtue of the presence of non-basepaired ribonucleotides at least in the antisense RNA sequence (J). Each of the above-mentioned frequencies of non-canonical basepairs such as G:U basepairs can be applied to dsRNA regions of less than 30 basepairs, but also to longer dsRNA regions. Clearly, a population of siRNA molecules having different numbers of non-canonical basepairs and/or non-basepaired ribonucleotides is generated by processing of a precursor RNA molecule, depending on the frequency and arrangement of the basepairs and ribonucleotides along the length of the dsRNA region.

In an embodiment, a dsRNA molecule of the invention, including the precursor RNA molecules such as hairpin RNA and ledRNA molecules and a “double-stranded product RNA molecule (P)” of the invention, comprises 18 basepairs, wherein at least one basepair, or only one basepair, of the 18 basepairs is a non-canonical basepair, preferably a G:U basepair where the G is in the sense RNA sequence (H) and the U is in the antisense RNA sequence (J), more preferably the G is ribonucleotide 19 of the sense RNA sequence (H) and the U is ribonucleotide 1 of the antisense RNA sequence (J). In an embodiment, the dsRNA region comprises 18 basepairs, wherein at least 2 basepairs, or exactly 2 basepairs, of the 18 basepairs are non-canonical basepairs. In an embodiment, the dsRNA region comprises 18 basepairs, wherein at least 3 basepairs, or exactly 3 basepairs, of the 18 basepairs are non-canonical basepairs. In an embodiment, the dsRNA region comprises 18 basepairs, wherein at least 4 basepairs, or exactly 4 basepairs, of the 18 basepairs are non-canonical basepairs. In an embodiment, the dsRNA region comprises 18 basepairs, wherein at least 5 basepairs, or exactly 5 basepairs, of the 18 basepairs are non-canonical basepairs. In an embodiment, the dsRNA region comprises 18 basepairs, wherein at least 6 basepairs, or exactly 6 basepairs, of the 18 basepairs are non-canonical basepairs. In an embodiment, the dsRNA region comprises 18 basepairs, wherein at least 7 basepairs, or exactly 7 basepairs, of the 18 basepairs are non-canonical basepairs. In an embodiment, the dsRNA region comprises 18 basepairs, wherein at least 8 basepairs, or exactly 8 basepairs, of the 18 basepairs are non-canonical basepairs. In an embodiment, the dsRNA region comprises 18 basepairs, wherein at least 9 basepairs, or exactly 9 basepairs, of the 18 basepairs are non-canonical basepairs. In each of these embodiments, it is preferred that a maximum of 10 of the 18 basepairs in the dsRNA region are non-canonical basepairs, more preferably a maximum of 9 of the basepairs in the dsRNA region are non-canonical basepairs, still more preferably a maximum of 8 of the basepairs in the dsRNA region are non-canonical basepairs, even still more preferably a maximum of 7 of the basepairs in the dsRNA region are non-canonical basepairs, and most preferably a maximum of 6 of the basepairs in the dsRNA region are non-canonical basepairs. Preferably, in the above embodiments, the non-canonical basepairs comprise at least one G:U basepair, more preferably all of the non-canonical basepairs are G:U basepairs. Also preferred, more of the G:U basepairs have the G ribonucleotide in the sense sequence (H) and the U ribonucleotide in the antisense sequence (J) than the converse. In an embodiment, at least twice as many, or at least three times as many, of the G:U basepairs have the G ribonucleotide in the sense sequence (H), or more preferred all of the G:U basepairs have the G ribonucleotide in the sense sequence (H). In each of the embodiments described in this paragraph, in the context of the Δ22, Δ23 and Δ24 modifications, the 18 basepairs are not contiguous by virtue of the presence of non-basepaired ribonucleotides at least in the antisense RNA sequence (J).

In an embodiment, the ratio of canonical to non-canonical basepairs in the dsRNA region is between 2.5:1 and 3.5:1, for example about 3:1. In an embodiment, the ratio of canonical to non-canonical basepairs in the dsRNA region is between 3.5:1 and 4.5:1, for example about 4:1. In an embodiment, the ratio of canonical to non-canonical basepairs in the dsRNA region is between 4.5:1 and 5.5:1, for example about 5:1. In an embodiment, the ratio of canonical to non-canonical basepairs in the dsRNA region is between 5.5:1 and 6.5:1, for example about 6:1. Different dsRNA regions in the RNA molecule may have different ratios.

In the above embodiments, the non-canonical basepairs in the dsRNA region(s) of the RNA molecule are preferably all G:U basepairs. In an embodiment, preferably for dsRNA region(s) of greater than 100 basepairs in length, at least 95% of the non-canonical basepairs are G:U basepairs. In an embodiment, at least 90% of the non-canonical basepairs are G:U basepairs. In an embodiment, between 90 and 95% of the non-canonical basepairs are G:U basepairs. For example, if there are 10 non-canonical basepairs, at least 9 (90%) are G:U basepairs.

In an example of the above embodiments, there are at least 3 G:U basepairs in one or more or all dsRNA regions of the RNA molecule. In another example, there are at least 4, 5, 6, 7, 8, 9 or 10 G:U basepairs. In another example, there are at least between 3 and 10 G:U basepairs. In another example, there are at least between 5 and 10 G:U basepairs. These numbers can also be applied to the product RNA molecules (P) of the invention.

For all of the above embodiments referring to G:U basepairs, the G:U basepairs can be readily achieved through the use of C to T or preferably A to G substitutions in the sense sequence of the DNA construct encoding the RNA molecule, or A to G or preferably C to T substitutions in the antisense sequence, or both, relative to a corresponding wild-type target sequence or its complement, as exemplified herein. It is understood that the C to T substitutions are in the context of a DNA construct encoding the precursor RNA molecule. This results in the substitution of C to U in the precursor RNA molecules and subsequent product siRNAs. In all of these embodiments, it is most preferred that the antisense sequence is fully complementary to a region of a wild-type target RNA molecule i.e. lacks substitutions relative to the complementary region to a wild-type target sequence. In this context, “fully complementary” means that all ribonucleotides of the antisense sequence basepair with the target RNA molecule by canonical basepairing. Alternatively, in an embodiment, all ribonucleotides of the antisense sequence basepair with the target RNA molecule but some of the basepairs are G:U basepairs. This can be readily achieved through the use of A to G or preferably C to T substitutions in the antisense sequence of the DNA construct.

In an embodiment, a precursor RNA molecule of the invention does not comprise a non-canonical basepair at the base of a loop of the molecule. Alternatively, a precursor RNA molecule of the invention comprises a G:U basepair at the base of a loop of the molecule. In another embodiment, one, two, three, four, five or more or all of the non-canonical basepairs, preferably G:U basepairs, are each flanked by canonical basepairs. In an embodiment, the dsRNA region comprises multiple contiguous G:U basepairs, for example 2 or even 3 contiguous G:U basepairs. Alternatively, the dsRNA region lacks contiguous G:U basepairs. In an embodiment, the dsRNA region comprises one or more G:U basepairs which are adjacent to a non-basepaired ribonucleotide in the antisense sequence, forming a bulge which may be advantageously recognised by a ribonuclease such as Drosha.

The present invention also provides, applicable to all of the embodiments described above, for a combination of the asymmetric RNA design with the Δ22, Δ23 or Δ24 modifications as described herein, with the presence of non-canonical basepairing such as G:U basepairs in at least one dsRNA region of the precursor RNA molecule, preferably all double-stranded regions. In preferred examples of each of the above embodiments which are asymmetric RNA molecules, at least part of the dsRNA region, preferably the whole dsRNA region, comprises one or more non-basepaired ribonucleotides, in either the sense sequence or the antisense sequence, or both, provided that more non-basepaired ribonucleotides are present in the antisense sequence than in the sense sequence of the dsRNA region. In each of the above embodiments, it is preferred that the non-basepaired ribonucleotides are distributed along the dsRNA region in a regularly-spaced manner, for example one non-basepaired ribonucleotide, or one more non-basepaired ribonucleotide in the antisense sequence than the sense sequence, on average about every 1 in 22, or 2 in 23, or 3 in 24 ribonucleotides. Alternatively, for each of the above embodiments, at least part of the sense sequence of the dsRNA region such as at least 50 or 100 ribonucleotides in length, even the whole sense sequence of the dsRNA region, does not comprise non-basepaired ribonucleotides. In that case, the only non-basepaired ribonucleotides are in the antisense sequence of the dsRNA region. Again, in embodiments having non-canonical basepairing as described herein, both the sense and antisense sequences in the dsRNA lack any non-basepaired ribonucleotides i.e. a symmetric dsRNA which is fully basepaired.

In an embodiment, the chimeric RNA molecule comprises at least one plant DCL-1 cleavage site.

Each of the embodiments in this section “Non-canonical basepairing” can be applied to reduce the activity of target RNA molecules in eukaryotic cells, preferably in a plant, fungal or nematode cell, or a pest or pathogen thereof. They can also be applied in an arthropod cell such as an insect cell or other non-vertebrate animal cell, or in a vertebrate animal cell. In each embodiment, the region of the target RNA molecule corresponding to the dsRNA region may be in the range 31-50 ribonucleotides in length, or at least 44, at least 50, at least 60, at least 65, at least 70, at least 80, at least 86, at least 90 or at least 100 contiguous ribonucleotides in length. The length of region may also be at least 150, at least 200, at least 250 or at least 300 ribonucleotides, to a maximum of the full length of the target RNA molecule. A preferred length for plant, fungal or nematode target RNA molecules is in the range 100-600 ribonucleotides.

In an embodiment, the target RNA molecule is not a viral RNA molecule. Alternatively, the target RNA molecule is a viral RNA molecule such as, for example, an RNA molecule or RNA transcript of a virus that infects a plant.

### 5′ Leader, 3′ Trailer and Intervening Sequences Such as Loop Sequences

In an embodiment, the RNA molecule of the invention such as a hairpin RNA molecule or a ledRNA molecule comprises a 5′ leader sequence, or 5′ extension sequence, which may arise as a result of transcription from a promoter in the genetic construct, from the start site of transcription to the beginning of the polynucleotide encoding the remainder of the RNA molecule. It is preferred that this 5′ leader sequence or 5′ extension sequence is relatively short compared to the remainder of the molecule, and it may be removed from the RNA molecule post-transcriptionally, for example by RNase treatment. For example, the molecule can be designed so that transcription starts at a defined starting nucleotide which corresponds to the first 5′ ribonucleotide or the second 5′ ribonucleotide, for instance with in vitro transcription, whereby the precursor RNA molecule lacks 5′ leader. If present, 5′ leader sequence or 5′ extension sequence may be mostly non-basepaired, or it may contain one or more stem-loop structures. In this embodiment, 5′ leader sequence consists of a sequence of ribonucleotides which is covalently linked to the first 5′ ribonucleotide if the second RNA component is linked to the first 3′ ribonucleotide or to the second 5′ ribonucleotide if the second RNA component is linked to the first 5′ ribonucleotide. In an embodiment, the 5′ leader sequence is at least 10, at least 20, at least 30, at least 100, at least 200 ribonucleotides long, preferably to a maximum length of 250 ribonucleotides. In another embodiment, 5′ leader sequence is at least 50 ribonucleotides long. In an embodiment, the 5′ leader sequence can act as an extension sequence for amplification of the RNA molecule via a suitable amplification reaction. In an embodiment, the extension sequence may facilitate amplification via polymerase.

In another embodiment, the RNA molecule comprises a 3′ trailer sequence or 3′ extension sequence which may arise as a result of transcription continuing until a transcription termination or polyadenylation signal in the construct encoding the RNA molecule. Alternatively, the molecule can be designed so that transcription ends at a defined nucleotide which corresponds to the first 3′ ribonucleotide or the second 3′ ribonucleotide, for instance with in vitro transcription, whereby the precursor RNA molecule lacks a 3′ trailer. If present, 3′ trailer sequence or 3′ extension sequence may comprise a poly A tail. It is preferred that this 3′ trailer sequence or 3′ extension sequence is relatively short compared to the remainder of the molecule, and it may be removed from the RNA molecule post-transcriptionally, for example by RNase treatment. The 3′ trailer sequence or 3′ extension sequence may be mostly non-basepaired, or it may contain one or more stem-loop structures. In an embodiment of the ledRNA structure, the 3′ trailer sequence consists of a sequence of ribonucleotides which is covalently linked to the second 3′ ribonucleotide if the second RNA component is linked to the first 3′ ribonucleotide or to the first 3′ ribonucleotide if the second RNA component is linked to the first 5′ ribonucleotide. In an embodiment, 3′ leader sequence is at least 10, at least 20, at least 30, at least 100, at least 200 ribonucleotides long, preferably to a maximum length of 250 ribonucleotides. In another embodiment, 3′ leader sequence is at least 50 ribonucleotides long. In an embodiment, 3′ trailer sequence can act as an extension sequence for amplification of the RNA molecule via a suitable amplification reaction. For example, the extension sequence may facilitate amplification via polymerase.

In an embodiment, 5′ leader and 3′ trailer sequences are not directly covalently bound to each other. In an embodiment, 5′ leader and 3′ trailer sequences are separated by a nick. In an embodiment, 5′ leader and 3′ trailer sequences are ligated together to provide a RNA molecule with a closed structure. In another embodiment, the 5′ leader and 3′ trailer sequences are separated by a loop.

In an embodiment, the linking ribonucleotide sequence is less than 100 ribonucleotides in length. In an embodiment, the linking ribonucleotide sequence is less than 50 ribonucleotides in length. In an embodiment, the linking ribonucleotide sequence is less than 20 ribonucleotides in length. In an embodiment, the linking ribonucleotide sequence is less than 10 ribonucleotides in length. In an embodiment, the linking ribonucleotide sequence is less than 5 ribonucleotides in length. In an embodiment, the linking ribonucleotide sequence is between 2 and 100 ribonucleotides in length. In an embodiment, the linking ribonucleotide sequence is between 2 and 50 ribonucleotides in length. In an embodiment, the linking ribonucleotide sequence is between 2 and 20 ribonucleotides in length. In an embodiment, the linking ribonucleotide sequence is between 2 and 10 ribonucleotides in length. In an embodiment, the linking ribonucleotide sequence is between 2 and 5 ribonucleotides in length. In an embodiment, the ribonucleotides of the linking ribonucleotide sequence are not basepaired. In a preferred embodiment, the ribonucleotides of the linking ribonucleotide sequence are all basepaired within the ledRNA molecule, or all except for 1, 2 or 3 of the ribonucleotides are basepaired.

In an embodiment of the RNA molecule of the invention such as a hairpin RNA or a ledRNA molecule, one or more or all of 5′ leader sequence, 3′ trailer sequence or intervening linker sequence, or preferably one or more of the loop sequences, comprises an additional sequence which is related in sequence to the target RNA transcript i.e. comprises a sequence which is not basepaired in the RNA molecule and which is either identical to a region of the target RNA transcript (additional sense sequence) or identical to the complement of that region (additional antisense sequence), which region is either the same as, or different to, the region targeted by the first and/or second antisense sequences. In embodiments, the length of the additional sense sequence or additional antisense sequence is at least 50, at least 60, at least 70, at least 80, at least 90, at least 100, at least 150 or at least 200 ribonucleotides, and the degree of sequence identity is at least 90%, at least 95% or preferably 100% identical over that length either identical to the region of the target RNA transcript or to the complement of that region. Each of the above embodiments of the RNA molecules of the invention may be used to reduce the amount and/or activity of a target RNA transcript in a plant cell such as an endogenous plant RNA transcript or an RNA of a viral pathogen of a plant, or a fungal RNA transcript such as a plant-pathogenic fungus (PPF) or animal-pathogenic fungus, or an invertebrate animal RNA transcript such as a nematode transcript, particularly a plant-pathogenic nematode (PPN).

The term “loop” is used in the context of the present disclosure to refer to a loop structure in a precursor RNA molecule disclosed herein that is formed by a series of non-complementary ribonucleotides in a linking ribonucleotide sequence. This is typically in the context of a “stem-loop” structure. Loops generally follow a series of basepairs between the first and second RNA components or join a sense RNA sequence and an antisense RNA sequence. In an example, all loop ribonucleotides are non-basepaired, generally for shorter loops of 4-10 ribonucleotides. In other examples, some ribonucleotides can be complementary so long as these matches enable a loop structure to form. For example, at least 5%, at least 10%, at least 15% of loop ribonucleotides can be complementary. Examples of loops as contemplated herein include pseudoknots and tetraloops, for example sequences such as ANYA, CUYG, GNRA UMAC and UNCG, where “Y” refers to a pyrimidine ribonucleotide, “R” refers to a purine ribonucleotide, “M” refers to C or A, and “N” refers to any ribonucleotide.

In an embodiment, the precursor RNA molecule has only one loop i.e. forms a hairpin RNA structure, also referred to as a stem-loop structure. In another embodiment, the precursor RNA molecule comprises two loops, including for example, ledRNA molecules of the invention. In other embodiments, the RNA molecule comprises at least two, at least three, at least 4, at least 5, at least 6, at least 7, at least 8, at least 9, at least 10 loops, including the ledRNA molecules. For example, the precursor RNA molecule can comprise at least 4 loops.

Loops of various sizes are contemplated by the present disclosure. For example, loops can comprise at least 4, at least 5, at least 6, at least 7, at least 8, at least 9, at least 10, at least 11, or at least 12 ribonucleotides. In other embodiments, loops comprise at least 15, at least 20, at least 25, or at least 30 ribonucleotides. For example, one or all of the loop sequences can be longer than 20 ribonucleotides. In other embodiments, loops are much larger, comprising at least 50, at least 100, at least 150, at least 200, or at least 300 ribonucleotides. In an embodiment, loops comprise at least 160 ribonucleotides. In another embodiment, less preferred, loops can comprise at least 200, 500, 700, or 1,000 ribonucleotides provided that the loops do not interfere with the hybridisation of the sense and antisense RNA sequences. In an embodiment, each loop of the precursor RNA molecule comprises about the same number (+/−10%) of ribonucleotides. In an embodiment, the one or more loops are between 100 and 1,000 ribonucleotides in length. For example, loops may be between 600 and 1,000 ribonucleotides in length. For example, loops may be between 4 and 1,000 ribonucleotides in length. For example, loops preferably may be between 4 and 50 ribonucleotides in length. In another embodiment, each loop of the precursor RNA molecule comprises a different number of ribonucleotides. In another example, two or more of the loops of the precursor RNA molecule comprise about the same number (+/−10%) of ribonucleotides.

In another embodiment, one or more loops comprise an intron which can be spliced out of the RNA molecule after transcription in a eukaryotic cell. In an embodiment, the intron is from a plant gene. Exemplary introns include intron 3 of the maize alcohol dehydrogenase 1 (Adh1) (GenBank: AF044293), intron 4 of the soya beta-conglycinin alpha subunit (GenBank: AB051865); one of the introns of the pea rbcS-3A gene for the ribulose-1,5-bisphosphate carboxylase (RBC) small subunit (GenBank: X04333). Other examples of suitable introns are discussed in Mccullough and Schuler, (1997) and Smith et al. (2000). The invention therefore provides precursor RNA molecules which comprise an intron in the molecule, for example in a loop sequence, and the precursor RNA molecules after the intron has been spliced out. In a preferred embodiment, the precursor RNA molecule of the invention is encoded by a genetic construct that does not include a sequence encoding an intron i.e. the RNA molecule never had an intron.

In various examples, a loop may be at the end of a dsRNA stem which has at least 3, at least 4, at least 5, at least 6, at least 7, at least 8, at least 9, at least 10, at least 11, at least 12, at least 13, at least 14, at least 15, at least 16, at least 17, at least 18, or at least 19 consecutive basepairs, for example, canonical basepairs. In a preferred embodiment, the loop is adjacent to one or two G:U basepair(s) at the end of the dsRNA region, preferably with the U of the G:U basepair(s) being the first, or first two, ribonucleotides at the 5′ end of the antisense sequence forming the double-stranded “stem”.

In an embodiment, a loop of the RNA molecule of the invention comprises an RNA sequence which is complementary to a miRNA or other sRNA in the eukaryotic cell, where the RNA sequence is capable of binding or sequestering the miRNA or other sRNA, thereby capable of reducing its activity. The loop may comprise multiple such RNA sequences which may be the same or different, each being complementary to a miRNA or other sRNA in the cell.

In an embodiment, the precursor RNA molecule, for example a hairpin RNA, comprises a second sense or antisense sequence which comprises at least 100 ribonucleotides of a sequence from within the dsRNA region, referred to as a seed region, incorporated into the loop sequence or elsewhere in the precursor RNA molecule. That is, the seed region corresponds to a sequence of at least 100 nt from within the sense sequence or antisense sequence used to form the duplex of the RNA molecule. The inventors considered that the 22-mer antisense sRNAs produced by Dicer from the duplex region of the RNA molecules would hybridise to this seed region in the single-stranded loop region and thereby stimulate production of secondary siRNAs. The loop sequence may be chimeric in comprising a seed region as well as other sequences related to the target RNA molecule or its complement. The seed region could also be inserted into different regions of the RNA molecule, in particular attached to its 5′ or 3′ end rather than in the loop. Constructs encoding ledRNA molecules having the same type of modified loop sequences and modified duplex regions were also contemplated, with or without seed regions, including where the duplex regions of the first and second components targeted different target RNA transcripts and where the first and second loop sequences corresponded to regions from the different target transcripts.

All combinations of the features of the RNA molecules of the invention described herein are contemplated. For example, each of the above embodiments for the ledRNA structures may comprise one or more features described in embodiments for the asymmetric RNA molecules. Each of the above embodiments for the ledRNA structures may comprise one or more features described in the embodiments for the RNA molecules comprising non-canonical basepairs, preferably G:U basepairs. Each of the above embodiments for the ledRNA structures may comprise one or more features described in the embodiments for the asymmetric RNA molecules and one or more of the features described herein for the RNA molecules comprising non-canonical basepairs, preferably G:U basepairs.

### Sequence Relationship to a Region of a Target RNA Molecule

The RNA molecules of the invention are capable of reducing the activity of target RNA molecule(s) because of a degree of sequence identity to a region of the target RNA molecule. In an embodiment, the sense RNA sequence has substantial sequence identity to a region of the target RNA molecule, which identity may be to a sequence of less than 21 nucleotides in length. In an embodiment at least 15, at least 16, at least 17, at least 18, or at least 19 contiguous ribonucleotides, preferably at least 20 or 21 contiguous ribonucleotides, of the sense ribonucleotide sequence and the region of the target RNA molecule are at least 60%, at least 65%, at least 70%, at least 75%, at least 80%, at least 85%, at least 90%, at least 95%, at least 96%, at least 97%, at least 98%, or 99% identical in sequence. The less than 100% sequence identity allows for the presence of ribonucleotide deletions in the sense RNA sequence relative to the region of the target RNA molecule as may occur in the Δ22, Δ23 and Δ24 modifications, or ribonucleotide substitutions such as A to G and C to U substitutions in the sense RNA sequence to provide for G:U basepairs. In another embodiment, the at least 15, at least 16, at least 17, at least 18, at least 19 or at least 20 or 21 contiguous ribonucleotides of the sense ribonucleotide sequence and a region of a target RNA molecule are 100% identical. In an embodiment, at least the last 3, last 4, last 5, last 6, last 7, preferably the last 8 ribonucleotides at the 3′ end of the sense ribonucleotide sequence are 100% identical to the region of the target RNA molecule, with the remaining ribonucleotides being at least 60%, at least 65%, at least 70%, at least 75%, at least 80%, at least 85%, at least 90%, at least 95%, at least 96%, at least 97%, at least 98%, at least 99% identical to the target RNA molecule.

In an embodiment, the at least 21 contiguous ribonucleotides of the sense ribonucleotide sequence and a region of a target RNA molecule are at least 60%, at least 65%, at least 70%, at least 75%, at least 80%, at least 85%, at least 90%, at least 95%, at least 96%, at least 97%, at least 98%, or at least 99% identical. Again, in this embodiment, at least the last 3, last 4, last 5, last 6, last 7 or last 8 ribonucleotides of the sense ribonucleotide sequence can be 100% identical to the region of the target RNA molecule, with the remaining ribonucleotides being at least 60%, at least 65%, at least 70%, at least 75%, at least 80%, at least 85%, at least 90%, at least 95%, at least 96%, at least 97%, at least 98%, or at least 99% identical to the target RNA molecule.

In an embodiment, the antisense sequence has substantial sequence identity to the complement of a region of the target RNA molecule, which identity may be to a sequence of less than 22 ribonucleotides in length of the complement where the antisense sequence is 22 ribonucleotides in length, or less than 23 ribonucleotides where the antisense sequence is 23 ribonucleotides in length, or less than 24 ribonucleotides where the antisense sequence is 24 ribonucleotides in length. In an embodiment, at least 15, at least 16, at least 17, at least 18, at least 19, or at least 20 contiguous ribonucleotides, preferably at least 21 contiguous ribonucleotides, of the 22 nt antisense ribonucleotide sequence, preferably at least 22 contiguous ribonucleotides or the 23 ribonucleotides of the 23 nt antisense ribonucleotide sequence, or at least 22 contiguous ribonucleotides, preferably at least 23 contiguous ribonucleotides or 24 ribonucleotides of the 24 nt antisense ribonucleotide sequence, and the complement of a region of a target RNA molecule are at least 60%, at least 65%, at least 70%, at least 75%, at least 80%, at least 85%, at least 90%, at least 95%, at least 96%, at least 97%, at least 98%, or 99% identical in sequence. In another embodiment, the at least 15, at least 16, at least 17, at least 18, at least 19, at least 20, at least 21, or all 22, 23 or 24 contiguous ribonucleotides of the antisense ribonucleotide sequence and the complement of the region of the target RNA molecule are 100% identical. In an embodiment, at least the first 3, first 4, first 5, first 6, or first 7 ribonucleotides from the 5′ end of the antisense ribonucleotide sequence, preferably at least all of ribonucleotides 2-8, are 100% identical to the complement of the region of the target RNA molecule, with the remaining ribonucleotides being at least 60%, at least 65%, at least 70%, at least 75%, at least 80%, at least 85%, at least 90%, at least 95%, at least 96%, at least 97%, at least 98%, at least 99% identical to the complement of the target RNA molecule. The less than 100% sequence identity allows for the presence of ribonucleotide insertions into the antisense RNA sequence relative to the complement of the region of the target RNA molecule as may occur in the Δ22, Δ23 and Δ24 modifications, or ribonucleotide substitutions such as A to G and C to U substitutions in the antisense RNA sequence to provide for G:U basepairs.

In an embodiment, the 22 contiguous ribonucleotides of the 22 nt antisense ribonucleotide sequence, or the 23 contiguous ribonucleotides of the 23 nt antisense ribonucleotide sequence, or the 24 contiguous ribonucleotides of the 24 nt antisense ribonucleotide sequence, and the complement of a region of the target RNA molecule are at least 60%, at least 65%, at least 70%, at least 75%, at least 80%, at least 85%, at least 90%, at least 95%, at least 96%, at least 97%, at least 98%, or at least 99% identical, preferably 100% identical. At least 95% sequence identity is preferred for target RNA molecules in a plant cell, insect cell, fungal cell or nematode cell, for example for antisense sequences of at least 44 ribonucleotides, or at least 68 ribonucleotides, or at least 90 or at least 100 ribonucleotides in length, whereas 60-95% sequence identity is acceptable for target RNA molecules in a vertebrate animal cell such as a mammalian animal cell. In an embodiment for all of these cell types, at least the first 3, first 4, first 5, first 6, or first 7 ribonucleotides, preferably at least all of ribonucleotides 2-8, are 100% identical to the complement of the region of the target RNA molecule, with the remaining ribonucleotides being at least 60%, at least 65%, at least 70%, at least 75%, at least 80%, at least 85%, at least 90%, at least 95%, at least 96%, at least 97%, at least 98%, or at least 99% identical to the complement of the target RNA molecule. In another, preferred embodiment, the 22 contiguous ribonucleotides of the 22 nt antisense ribonucleotide sequence, or the 23 contiguous ribonucleotides of the 23 nt antisense ribonucleotide sequence, or the 24 contiguous ribonucleotides of the 24 nt antisense ribonucleotide sequence and the complement of a region of a target RNA molecule are 100% identical. As understood in the art, the antisense RNA sequence does not require 100% sequence identity to the complement of the region of the target RNA molecule in order to be effective in reducing the expression or activity of the target RNA molecule, for example by translational repression. For target RNA molecules in plant, insect, nematode or fungal cells, however, 100% sequence identity to the complement is preferred.

In preferred embodiments, the sequence identity percentages recited in the previous two paragraphs for the antisense sequences apply to longer sequences for the complementary region of the target RNA molecule, for example over at least 30 contiguous ribonucleotides, at least 34 contiguous ribonucleotides, at least 40 contiguous ribonucleotides, at least 46 contiguous ribonucleotides, at least 50 contiguous ribonucleotides, or at least 60, at least 65, at least 70, at least 80, at least 84, at least 90 contiguous ribonucleotides, more preferably at least 100 contiguous ribonucleotides.

### Nucleic Acids Encoding RNA Molecules

One of skill in the art will appreciate from the foregoing description that the present disclosure also provides an isolated, or exogenous or recombinant, nucleic acid (polynucleotide) encoding one or more RNA molecules disclosed herein and the component parts thereof. The nucleic acid may be partially purified after expression in a host cell. The term “partially purified” is used to refer to an RNA molecule that has generally been separated from the lipids, nucleic acids, other peptides, and other contaminating molecules with which it is associated in a host cell. Preferably, the partially purified polynucleotide is at least 60% free, more preferably at least 75% free, and more preferably at least 90% free from other components with which it is associated.

A polynucleotide according to the present invention is a heterologous polynucleotide. The term “heterologous polynucleotide” is well understood in the art and refers to a polynucleotide which is not endogenous to a cell, or is a polynucleotide in which the native sequence has been altered, or a native polynucleotide whose expression is quantitatively altered as a result of a manipulation of the cell by recombinant DNA techniques. The polynucleotide may be a “chimeric polynucleotide” in that it comprises components which are arranged in a manner not found in nature. In an embodiment, the chimeric polynucleotide is expressed in a cell from a heterologous promoter i.e. a promoter that is not operably linked, in nature, to the expressed sequence. In an overlapping embodiment, the chimeric polynucleotide comprises a sequence in a sense orientation and a second sequence in an antisense orientation, with respect to a target gene sequence, which do not naturally occur together in a polynucleotide. That is, the polynucleotide is a genetic construct, artificially made.

In an embodiment therefore, a polynucleotide according to the present disclosure is a synthetic polynucleotide. For example, the polynucleotide may be produced using techniques that do not require pre-existing nucleic acid sequences such as DNA printing and oligonucleotide synthesis.

In an embodiment, a polynucleotide disclosed herein encodes an RNA precursor molecule comprising an intron in at least one loop sequence which is capable of being spliced out during transcription of the polynucleotide in a host cell. In an embodiment, the intron sequence thereby acts as a spacer in the polynucleotide between sense and antisense sequences. In another example, a loop sequence comprises two introns, or each loop sequence comprises an intron. Alternatively, the polynucleotide does not comprise an intron in its loop sequences or within its transcribed region. The present disclosure also provides an expression construct such as a DNA construct comprising a polynucleotide of the invention operably linked to a promoter. In an example, such polynucleotides and/or expression constructs are provided in a cell or non-human organism. In an embodiment, the polynucleotide is stably integrated into the genome of the cell or non-human organism. Various examples of suitable expression constructs, promoters and cells comprising the same are discussed herein.

Synthesis of RNA molecules according to the present disclosure can be achieved using various methods known in the art. The Examples section provides an example of in vitro (cell-free) synthesis. In this example, constructs encoding RNA molecules disclosed herein are restricted at the 3′ end of a region to be transcribed, purified and quantified, and then transcribed. Alternatively, RNA synthesis can be achieved in bacterial culture following transformation of E. coli HT115 cells and induction of RNA synthesis using the T7, IPTG system, or in yeast cells or other microbial cells, either prokaryotic or eukaryotic.

### Recombinant Vectors

One embodiment of the present invention includes a recombinant vector, which comprises, or encodes, at least one RNA molecule defined herein and is capable of delivering the RNA molecule into a host cell or expressing the RNA molecule in the cell. Recombinant vectors include expression vectors. Recombinant vectors contain a heterologous polynucleotide sequence, that is, a polynucleotide sequence that is not naturally found operably linked to a sequence encoding an RNA molecule defined herein. The sequences may be derived from different species, or more often the sequence encoding the RNA molecule does not occur naturally. The vector can be either RNA or DNA, and typically is a viral vector, derived from a virus, or a plasmid.

Various viral vectors can be used to deliver and mediate expression of an RNA molecule according to the present disclosure. The choice of viral vector will generally depend on various parameters, such as the cell or tissue targeted for delivery, transduction efficiency of the vector and pathogenicity. In an example, a DNA form of the viral vector integrates into host genome (e.g. lentiviruses). In another example, the viral vector persists in the cell nucleus predominantly as an extrachromosomal episome (e.g. adenoviruses). Examples of these types of viral vectors include oncoretroviruses, lentiviruses, adeno-associated virus, adenoviruses, herpes viruses and retroviruses. In plant cells, the viral vectors may be derived from Gemini viruses, as exemplified herein.

Plasmid vectors typically include additional nucleic acid sequences that provide for easy selection, amplification, and transformation of the expression cassette in prokaryotic cells, e.g., pUC-derived vectors, pGEM-derived vectors or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells, all well known in the art.

“Operably linked” as used herein, refers to a functional relationship between two or more nucleic acid (e.g., DNA) segments. Typically, it refers to the functional relationship of a transcriptional regulatory element (promoter or transcription terminator) to a transcribed sequence. For example, a promoter is operably linked to a coding sequence of an RNA molecule defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements such as enhancers need not be physically contiguous or located in close proximity to the coding sequences whose transcription they enhance.

When there are multiple promoters present, each promoter may independently be the same or different.

To facilitate identification of transformants, the recombinant vector desirably comprises a selectable or screenable marker gene. By “marker gene” is meant a gene that imparts a distinct phenotype to cells expressing the marker gene and thus, allows such transformed cells to be distinguished from cells that do not have the marker. A selectable marker gene confers a trait for which one can “select” based on resistance to a selective agent (e.g., a herbicide, antibiotic). A screenable marker gene (or reporter gene) confers a trait that one can identify through observation or testing, that is, by “screening” (e.g., β-glucuronidase, luciferase, GFP or other enzyme activity not present in untransformed cells). Exemplary selectable markers for selection of plant transformants include, but are not limited to, a hyg gene which encodes hygromycin B resistance; a neomycin phosphotransferase (nptII) gene conferring resistance to kanamycin, paromomycin; a glutathione-S-transferase gene from rat liver conferring resistance to glutathione derived herbicides as for example, described in EP 256223; a glutamine synthetase gene conferring, upon overexpression, resistance to glutamine synthetase inhibitors such as phosphinothricin as for example, described in WO 87/05327; an acetyltransferase gene from Streptomyces viridochromogenes conferring resistance to the selective agent phosphinothricin as for example, described in EP 275957; a gene encoding a 5-enolpyruvyl-shikimate-3-phosphate synthase (EPSPS) conferring tolerance to N-phosphonomethylglycine as for example, described by Hinchee et al. (1988); a bar gene conferring resistance against bialaphos as for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS) which confers resistance to imidazolinone, sulfonylurea, or other ALS-inhibiting chemicals (EP 154,204); a mutated anthranilate synthase gene that confers resistance to 5-methyl tryptophan; or a dalapon dehalogenase gene that confers resistance to the herbicide.

Preferably, the recombinant vector, or part thereof, is stably incorporated into the genome of the cell such as the plant cell. Accordingly, the recombinant vector may comprise appropriate elements which allow the vector to be incorporated into the genome, or into a chromosome of the cell.

### Expression Vector

As used herein, an “expression vector” is a DNA vector that is capable of transforming a host cell and of effecting expression of an RNA molecule defined herein. Expression vectors of the present invention contain regulatory sequences such as transcription control sequences, translation control sequences, origins of replication, and other regulatory sequences that are compatible with the host cell and that control the expression of a RNA molecule according to the present disclosure. In particular, expression vectors of the present invention include transcription control sequences. Transcription control sequences are sequences which control the initiation, elongation, or termination of transcription. Particularly important transcription control sequences are those which control transcription initiation such as promoter, enhancer, operator and repressor sequences. The choice of the regulatory sequences used depends on the target organism such as a plant and/or target organ or tissue of interest. Such regulatory sequences may be obtained from any eukaryotic organism such as plants or plant viruses, or may be chemically synthesised.

Exemplary vectors suitable for stable transfection of plant cells or for the establishment of transgenic plants have been described in for example, Pouwels et al., Cloning Vectors: A Laboratory Manual, 1985, supp. 1987, Weissbach and Weissbach, Methods for Plant Molecular Biology, Academic Press, 1989, and Gelvin et al., Plant Molecular Biology Manual, Kluwer Academic Publishers, 1990. Typically, plant expression vectors include for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, a transcription termination site, and/or a polyadenylation signal.

Vectors of the invention can also be used to produce RNA molecules defined herein in a cell-free expression system, such systems are well known in the art.

In an example, a polynucleotide encoding an RNA molecule according to the present disclosure is operably linked to a promoter capable of directing expression of the RNA molecule in a host cell. In an example, the promoter functions in vitro, such as for example a T7 RNA polymerase or SP6 RNA polymerase promoter. In an embodiment, the promoter is an RNA polymerase III promoter. In another embodiment, the promoter is an RNA polymerase II promoter. However, the choice of promoter can depend on the target organism such as a plant, insect and/or target organ or tissue of interest. Exemplary mammalian promoters include CMV, EF1a, SV40, PGK1, Ubc, human beta actin, CAG, TRE, UAS, CaMKIIa, CAL1, 10, TEF1, GDS, ADH1, CaMV35S, Ubi, H1 and U6. Exemplary insect promoters include Ac5 and polyhedron promoters. A number of constitutive promoters that are active in plant cells have also been described. Suitable promoters for constitutive expression in plants include, but are not limited to, the cauliflower mosaic virus (CaMV) 35S promoter, the Figwort mosaic virus (FMV) 35S, the light-inducible promoter from the small subunit (SSU) of the ribulose-1,5-bisphosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α/β binding protein gene promoter. These promoters have been used to create DNA vectors that have been expressed in plants, see for example, WO 84/02913. All of these promoters have been used to create various types of plant-expressible recombinant DNA vectors.

For the purpose of expression in source tissues of the plant such as the leaf, seed, root or stem, it is preferred that the promoters utilised in the present invention have relatively high expression in these specific tissues. For this purpose, one may choose from a number of promoters for genes with tissue- or cell-specific, or -enhanced expression. Examples of such promoters reported in the literature include, the chloroplast glutamine synthetase GS2 promoter from pea, the chloroplast fructose-1,6-biphosphatase promoter from wheat, the nuclear photosynthetic ST-LS1 promoter from potato, the serine/threonine kinase promoter and the glucoamylase (CHS) promoter from Arabidopsis thaliana. Also reported to be active in photosynthetically active tissues are the ribulose-1,5-bisphosphate carboxylase promoter from eastern larch (Larix laricina), the promoter for the Cab gene, Cab6, from pine, the promoter for the Cab-1 gene from wheat, the promoter for the Cab-1 gene from spinach, the promoter for the Cab 1R gene from rice, the pyruvate, orthophosphate dikinase (PPDK) promoter from Zea mays, the promoter for the tobacco Lhcb1*2 gene, the Arabidopsis thaliana Suc2 sucrose-H³⁰ symporter promoter, and the promoter for the thylakoid membrane protein genes from spinach (PsaD, PsaF, PsaE, PC, FNR, AtpC, AtpD, Cab, RbcS). Other promoters for the chlorophyll α/β-binding proteins may also be utilized in the present invention such as the promoters for LhcB gene and PsbP gene from white mustard (Sinapis alba).

A variety of plant gene promoters that are regulated in response to environmental, hormonal, chemical, and/or developmental signals, also can be used for expression of polynucleotides of the invention in plant cells, including promoters regulated by (1) heat, (2) light (e.g., pea RbcS-3A promoter, maize RbcS promoter), (3) hormones such as abscisic acid, (4) wounding (e.g., WunI), or (5) chemicals such as methyl jasmonate, salicylic acid, steroid hormones, alcohol, Safeners (WO 97/06269), or it may also be advantageous to employ (6) organ-specific promoters.

As used herein, the term “plant storage organ specific promoter” refers to a promoter that preferentially, when compared to other plant tissues, directs gene transcription in a storage organ of a plant. For the purpose of expression in sink tissues of the plant such as the tuber of the potato plant, the fruit of tomato, or the seed of soybean, canola, cotton, Zea mays, wheat, rice, and barley, it is preferred that the promoters utilized in the present invention have relatively high expression in these specific tissues. The promoter for β-conglycinin or other seed-specific promoters such as the napin, zein, linin and phaseolin promoters, can be used. Root specific promoters may also be used. An example of such a promoter is the promoter for the acid chitinase gene. Expression in root tissue could also be accomplished by utilising the root specific subdomains of the CaMV 35S promoter that have been identified.

In an embodiment, the promoter directs expression in tissues and organs in which lipid biosynthesis take place. Such promoters may act in seed development at a suitable time for modifying lipid composition in seeds. Preferred promoters for seed-specific expression include: 1) promoters from genes encoding enzymes involved in lipid biosynthesis and accumulation in seeds such as desaturases and elongases, 2) promoters from genes encoding seed storage proteins, and 3) promoters from genes encoding enzymes involved in carbohydrate biosynthesis and accumulation in seeds. Seed specific promoters which are suitable are, the oilseed rape napin gene promoter (U.S. Pat. No. 5,608,152), the Vicia faba USP promoter (Baumlein et al., 1991), the Arabidopsis oleosin promoter (WO 98/45461), the Phaseolus vulgaris phaseolin promoter (U.S. Pat. No. 5,504,200), the Brassica Bce4 promoter (WO 91/13980), or the legumin B4 promoter (Baumlein et al., 1992), and promoters which lead to the seed-specific expression in monocots such as maize, barley, wheat, rye, rice and the like. Notable promoters which are suitable are the barley lpt2 or lpt1 gene promoter (WO 95/15389 and WO 95/23230), or the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene, the rye secalin gene). Other promoters include those described by Broun et al. (1998), Potenza et al. (2004), US20070192902 and US20030159173. In an embodiment, the seed specific promoter is preferentially expressed in defined parts of the seed such as the cotyledon(s) or the endosperm. Examples of cotyledon specific promoters include, but are not limited to, the FP1 promoter (Ellerstrom et al., 1996), the pea legumin promoter (Perrin et al., 2000), and the bean phytohemagglutnin promoter (Perrin et al., 2000). Examples of endosperm specific promoters include, but are not limited to, the maize zein-1 promoter (Chikwamba et al., 2003), the rice glutelin-1 promoter (Yang et al., 2003), the barley D-hordein promoter (Horvath et al., 2000) and wheat HMW glutenin promoters (Alvarez et al., 2000). In a further embodiment, the seed specific promoter is not expressed, or is only expressed at a low level, in the embryo and/or after the seed germinates.

In another embodiment, the plant storage organ specific promoter is a fruit specific promoter. Examples include, but are not limited to, the tomato polygalacturonase, E8 and Pds promoters, as well as the apple ACC oxidase promoter (for review, see Potenza et al., 2004). In a preferred embodiment, the promoter preferentially directs expression in the edible parts of the fruit, for example the pith of the fruit, relative to the skin of the fruit or the seeds within the fruit.

In an embodiment, the inducible promoter includes the Aspergillus nidulans alc system. Examples of inducible expression systems which can be used instead of the Aspergillus nidulans alc system are described in a review by Padidam (2003) and Corrado and Karali (2009). In another embodiment, the inducible promoter is a safener inducible promoter such as, for example, the maize In2-1 or In2-2 promoter (Hershey and Stoner, 1991), the safener inducible promoter is the maize GST-27 promoter (Jepson et al., 1994), or the soybean GH2/4 promoter (Ulmasov et al., 1995).

In another embodiment, the inducible promoter is a senescence inducible promoter such as, for example, senescence-inducible promoter SAG (senescence associated gene) 12 and SAG 13 from Arabidopsis (Gan and Amasino, 1995) and LSC54 from Brassica napus (Buchanan-Wollaston, 1994). Such promoters show increased expression at about the onset of senescence of plant tissues, in particular the leaves.

For expression in vegetative tissue leaf-specific promoters, such as the ribulose biphosphate carboxylase (RBCS) promoters, can be used. For example, the tomato RBCS1, RBCS2 and RBCS3A genes are expressed in leaves and light grown seedlings (Meier et al., 1997). A ribulose bisphosphate carboxylase promoters expressed almost exclusively in mesophyll cells in leaf blades and leaf sheaths at high levels, described by Matsuoka et al. (1994), can be used. Another leaf-specific promoter is the light harvesting chlorophyll a/b binding protein gene promoter (see, Shiina et al., 1997). The Arabidopsis thaliana myb-related gene promoter (Atmyb5) described by Li et al. (1996), is leaf-specific. The Atmyb5 promoter is expressed in developing leaf trichomes, stipules, and epidermal cells on the margins of young rosette and cauline leaves, and in immature seeds. A leaf promoter identified in maize by Busk et al. (1997), can also be used.

The termination of transcription is accomplished by a 3′ non-translated DNA sequence operably linked in the expression vector to the RNA molecule of interest. For a Pol II type promoter, 3′ non-translated region of a recombinant DNA molecule contains a polyadenylation signal that functions to cause the addition of adenylate ribonucleotides to the 3′ end of the RNA. The 3′ non-translated region can be obtained from various genes that are expressed in plant cells. The nopaline synthase 3′ untranslated region, 3′ untranslated region from pea small subunit Rubisco gene, 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable. For a Pol III type promoter, a transcription terminator sequence typically includes a run of five or more T nucleotides.

### Transfer Nucleic Acids

Transfer nucleic acids can be used to deliver an exogenous polynucleotide to a cell and comprise one, preferably two, border sequences and one or more RNA molecules of interest. The transfer nucleic acid may or may not encode a selectable marker. Preferably, the transfer nucleic acid forms part of a binary vector in a bacterium, where the binary vector further comprises elements which allow replication of the vector in the bacterium, selection, or maintenance of bacterial cells containing the binary vector. Upon transfer to a eukaryotic cell, the transfer nucleic acid component of the binary vector is capable of integration into the genome of the eukaryotic cell or, for transient expression experiments, merely of expression in the cell.

As used herein, the term “extrachromosomal transfer nucleic acid” refers to a nucleic acid molecule that is capable of being transferred from a bacterium such as Agrobacterium sp., to a eukaryotic cell such as a plant leaf cell. An extrachromosomal transfer nucleic acid is a genetic element that is well-known as an element capable of being transferred, with the subsequent integration of a DNA sequence contained within its borders into the genome of the recipient cell. In this respect, a transfer nucleic acid is flanked, typically, by two “border” sequences, although in some instances a single border at one end can be used and the second end of the transferred nucleic acid is generated randomly in the transfer process. The DNA sequence encoding an RNA molecule of interest is typically positioned between the left border-like sequence and the right border-like sequence of a transfer nucleic acid. The DNA sequence contained within the transfer nucleic acid may be operably linked to a variety of different promoter and terminator regulatory elements that facilitate its expression, that is, transcription and/or translation of the RNA molecule. Transfer DNAs (T-DNAs) from Agrobacterium sp. such as Agrobacterium tumefaciens or Agrobacterium rhizogenes, and man-made variants/mutants thereof are probably the best characterized examples of transfer nucleic acids. Another example is P-DNA (“plant-DNA”) which comprises T-DNA border-like sequences from plants.

As used herein, “T-DNA” refers to a T-DNA of an Agrobacterium tumefaciens Ti plasmid or from an Agrobacterium rhizogenes Ri plasmid, or variants thereof which function for transfer of DNA into plant cells. The T-DNA may comprise an entire T-DNA including both right and left border sequences, but need only comprise the minimal sequences required in cis for transfer, that is, the right T-DNA border sequence. The T-DNAs of the invention have inserted into them, anywhere between the right and left border sequences (if present), the RNA molecule of interest. The sequences encoding factors required in trans for transfer of the T-DNA into a plant cell such as vir genes, may be inserted into the T-DNA, or may be present on the same replicon as the T-DNA, or preferably are in trans on a compatible replicon in the Agrobacterium host. Such “binary vector systems” are well known in the art.

As used herein, a “border” sequence of a transfer nucleic acid can be isolated from a selected organism such as a plant or bacterium, or be a man made variant/mutant thereof. The border sequence promotes and facilitates the transfer of the RNA molecule to which it is linked and may facilitate its integration in the recipient cell genome. In an embodiment, a border-sequence is between 10-80 bp in length. Border sequences from T-DNA from Agrobacterium sp. are well known in the art and include those described in Lacroix et al. (2008).

As used herein, the terms “transfection”, “transformation” and variations thereof are generally used interchangeably. “Transfected” or “transformed” cells may have been manipulated to introduce the polynucleotides or vectors encoding the RNA molecule(s) of the invention, including progeny cells derived from the introduction and comprising the polynucleotides encoding the RNA molecules.

### Recombinant Cells

The invention also provides a recombinant cell, for example, a recombinant bacterial cell, fungal cell, plant cell, insect cell or animal cell, which is a host cell comprising one or more or all of the precursor RNA molecule of the invention, the double-stranded RNA molecule of the invention, the population of multiple, different double-stranded RNA molecules of the invention, the polynucleotide of the invention, or the vector of the invention. Suitable cells of the invention include any cell that can be transformed with a polynucleotide encoding an RNA molecule or recombinant vector according to the present disclosure, for example a microorganism such as a bacterial or fungal cell. In an embodiment, the transformed host cell is dead. For example, the host cell may be killed after it was transformed or after it produced RNA molecules of the invention.

The recombinant cell may be a cell in culture, a cell in vitro, or in an organism such as for example, a plant, or in an organ such as, for example, a seed or a leaf. Preferably, the cell is in a plant, more preferably in the seed of a plant. In one embodiment, the recombinant cell is a non-human cell. Accordingly, in an example, the present disclosure relates to a non-human organism comprising one or more or all of the precursor RNA molecule of the invention, the double-stranded RNA molecule of the invention, the population of multiple, different double-stranded RNA molecules of the invention, the polynucleotide of the invention, or the vector of the invention.

In an embodiment, the cells are selected from arthropod cells such as insect, arachnid, or decapod cells, fungal cells and nematode cells.

Another example of a suitable host cell is an E. coli HT115 cell.

Host cells of the present disclosure can be any cell capable of expressing at least one RNA molecule described herein, and include bacterial, fungal (including yeast), parasite, arthropod, animal and plant cells. Examples of host cells include Escherichia, Saccharomyces, Spodoptera, Mycobacteria, Trichoplusia, Agrobacterium, BHK (baby hamster kidney) cells, MDCK cells, CRFK cells, CV-1 cells, COS (e.g., COS-7) cells, and Vero cells. Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells.

In an embodiment, the plant cell is a seed cell, in particular, a cell in a cotyledon or endosperm of a seed, or a leaf cell. In an embodiment, the cell is an animal cell. The animal cell may be of any type of animal such as, for example, a non-human animal cell, a non-human vertebrate cell, a non-human mammalian cell, a human cell such as a human cell in cell culture, a nematode cell, or cells of aquatic animals such as fish or crustacea, invertebrates, insects, etc. Examples of algal cells useful as host cells of the present invention include, for example, Chlamydomonas sp. (for example, Chlamydomonas reinhardtii), Dunaliella sp., Haematococcus sp., Chlorella sp., Thraustochytrium sp., Schizochytrium sp., and Volvox sp.

### Plants

The invention also provides a plant comprising one or more or all of the precursor RNA molecule of the invention, the double-stranded RNA molecule of the invention, the population of multiple, different double-stranded RNA molecules of the invention, the polynucleotide of the invention, the vector of the invention, or the cell of the invention. The term “plant” when used as a noun refers to whole plants, whilst the term “part thereof” refers to plant organs (e.g., leaves, stems, roots, flowers, fruit), single cells (e.g., pollen), seed, seed parts such as an embryo, endosperm, scutellum or seed coat, plant tissue such as vascular tissue, plant cells and progeny of the same. As used herein, plant parts comprise plant cells.

As used herein, the terms “in a plant” and “in the plant” in the context of a modification to the plant means that the modification has occurred in at least one part of the plant, including where the modification has occurred throughout the plant, and does not exclude where the modification occurs in only one or more but not all parts of the plant. For example, a tissue-specific promoter is said to be expressed “in a plant”, even though it might be expressed only in certain parts of the plant.

As used herein, the term “plant” is used in its broadest sense, including any organism in the Kingdom Plantae. It also includes red and brown algae as well as green algae. It includes, but is not limited to, any species of flowering plant, grass, crop or cereal (e.g., oilseed, maize, soybean), fodder or forage, fruit or vegetable plant, herb plant, woody plant or tree. It is not meant to limit a plant to any particular structure. It also refers to a unicellular plant (e.g., microalga). The term “part thereof” in reference to a plant refers to a plant cell and progeny of same, a plurality of plant cells, a structure that is present at any stage of a plant's development, or a plant tissue. Such structures include, but are not limited to, leaves, stems, flowers, fruits, nuts, roots, seed, seed coat, embryos. The term “plant tissue” includes differentiated and undifferentiated tissues of plants including those present in leaves, stems, flowers, fruits, nuts, roots, seed, for example, embryonic tissue, endosperm, dermal tissue (e.g., epidermis, periderm), vascular tissue (e.g., xylem, phloem), or ground tissue (comprising parenchyma, collenchyma, and/or sclerenchyma cells), as well as cells in culture (e.g., single cells, protoplasts, callus, embryos, etc.). Plant tissue may be in planta, in organ culture, tissue culture, or cell culture.

As used herein, the term “vegetative tissue” or “vegetative plant part” is any plant tissue, organ or part other than organs for sexual reproduction of plants. The organs for sexual reproduction of plants are specifically seed-bearing organs, flowers, pollen, fruits and seeds. Vegetative tissues and parts include at least plant leaves, stems (including bolts and tillers but excluding the heads), tubers and roots, but excludes flowers, pollen, seed including the seed coat, embryo and endosperm, fruit including mesocarp tissue, seed-bearing pods and seed-bearing heads. In one embodiment, the vegetative part of the plant is an aerial plant part. In another or further embodiment, the vegetative plant part is a green part such as a leaf or stem.

A “genetically modified plant” or variations thereof refers to a plant that contains a genetic change not found in a wild-type plant of the same species, variety or cultivar. A “transgenic plant” or variations thereof refers to a plant that contains a transgene not found in a wild-type plant of the same species, variety or cultivar. Genetically modified/transgenic plants as defined in the context of the present invention include plants and their progeny which have been genetically modified using recombinant techniques to cause production of at least one RNA molecule defined herein in the desired plant or part thereof. Transgenic plant parts has a corresponding meaning.

The terms “seed” and “grain” are used interchangeably herein. “Grain” refers to mature grain such as harvested grain or grain which is still on a plant but ready for harvesting, but can also refer to grain after imbibition or germination, according to the context. “Developing seed” as used herein refers to a seed prior to maturity, typically found in the reproductive structures of the plant after fertilisation or anthesis, but can also refer to such seeds prior to maturity which are isolated from a plant.

As used herein, the term “plant storage organ” refers to a part of a plant specialized to store energy in the form of for example, proteins, carbohydrates, lipid. Examples of plant storage organs are seed, fruit, tuberous roots, and tubers. A preferred plant storage organ of the invention is seed.

As used herein, the term “phenotypically normal” refers to a genetically modified plant or part thereof, for example a transgenic plant, or a storage organ such as a seed, tuber or fruit of the invention not having a significantly reduced ability to grow and reproduce when compared to an unmodified plant or part thereof. Preferably, the biomass, growth rate, germination rate, storage organ size, seed size and/or the number of viable seeds produced is not less than 90% of that of a plant lacking said recombinant polynucleotide when grown under identical conditions.

Plants provided by or contemplated for use in the practice of the present invention include both monocotyledons and dicotyledons. In preferred embodiments, the plants of the present invention are crop plants (for example, cereals and pulses, maize, wheat, potatoes, rice, sorghum, millet, cassava, barley) or legumes such as soybean, beans or peas. The plants may be grown for production of edible roots, tubers, leaves, stems, flowers or fruit. The plants may be vegetable plants whose vegetative parts are used as food. The plants of the invention may be: Acrocomia aculeata (macauba palm), Arabidopsis thaliana, Aracinis hypogaea (peanut), Astrocaryum murumuru (murumuru), Astrocaryum vulgare (tucumã), Attalea geraensis (Indaiá-rateiro), Attalea humilis (American oil palm), Attalea oleifera (andaiá), Attalea phalerata (uricuri), Attalea speciosa (babassu), Avena sativa (oats), Beta vulgaris (sugar beet), Brassica sp. such as Brassica carinata, Brassica juncea, Brassica napobrassica, Brassica napus (canola), Camelina sativa (false flax), Cannabis sativa (hemp), Carthamus tinctorius (safflower), Caryocar brasiliense (pequi), Cocos nucifera (Coconut), Crambe abyssinica (Abyssinian kale), Cucumis melo (melon), Elaeis guineensis (African palm), Glycine max (soybean), Gossypium hirsutum (cotton), Helianthus sp. such as Helianthus annuus (sunflower), Hordeum vulgare (barley), Jatropha curcas (physic nut), Joannesia princeps (arara nut-tree), Lemna sp. (duckweed) such as Lemna aequinoctialis, Lemna disperma, Lemna ecuadoriensis, Lemna gibba (swollen duckweed), Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Licania rigida (oiticica), Linum usitatissimum (flax), Lupinus angustifolius (lupin), Mauritia flexuosa (buriti palm), Maximiliana maripa (inaja palm), Miscanthus sp. such as Miscanthus x giganteus and Miscanthus sinensis, including hybrids, Nicotiana sp. (tabacco) such as Nicotiana tabacum or Nicotiana benthamiana, Oenocarpus bacaba (bacaba-do-azeite), Oenocarpus bataua (patauã), Oenocarpus distichus (bacaba-de-leque), Oryza sp. (rice) such as Oryza sativa and Oryza glaberrima, Panicum virgatum (switchgrass), Paraqueiba paraensis (mari), Persea amencana (avocado), Pongamia pinnata (Indian beech), Populus trichocarpa, Ricinus communis (castor), Saccharum sp. (sugarcane), Sesamum indicum (sesame), Solanum tuberosum (potato), Sorghum sp. such as Sorghum bicolor, Sorghum vulgare, Theobroma grandiforum (cupuassu), Trifolium sp., Trithrinax brasiliensis (Brazilian needle palm), Triticum sp. (wheat) such as Triticum aestivum, Zea mays (corn), alfalfa (Medicago sativa), rye (Secale cerale), sweet potato (Lopmoea batatus), cassava (Manihot esculenta), coffee (Cofea spp.), pineapple (Anana comosus), citris tree (Citrus spp.), cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia) and almond (Prunus amygdalus). For example, plants of the disclosure may be Nicotiana benthamiana, or plants other than Nicotiana benthamiana or Nicotiana tabacum.

Other preferred plants include C4 grasses such as, in addition to those mentioned above, Andropogon gerardi, Bouteloua curtipendula, B. gracilis, Buchloe dactyloides, Schizachyrium scoparium, Sorghastrum nutans, Sporobolus cryptandrus; C3 grasses such as Elymus canadensis, the legumes Lespedeza capitata and Petalostemum villosum, the forb Aster azureus; and woody plants such as Quercus ellipsoidalis and Q. macrocarpa. Other preferred plants include C3 grasses.

Alternatively, the plant to be treated with the RNA molecules of the invention is a weed in order to down-regulate genes that are important for, for example, survival, growth, development, reproduction and/or seed dispersal of the weed. Weeds are a major contributor to yield loss and reduction in quality in agriculture, competing with the crop for resources like light, water and nutrients. This competition along with the cost of weed management strategies like tillage and herbicides is responsible for the economic impact of weeds, which reaches into the billions annually. Over 2,700 plant species have now been documented as weeds (Lazarides et al., 1997) and over 370 have been declared to be noxious by State and Territory governments in Australia. Plant genera or species that are weeds that may be treated with the RNA molecules of the invention include the following: Annual ryegrass (Lolium rigidum), the genus Echinochloa such as awnless barnyard grass (Echinochloa colona) or barnyard grass (Echinochloa crus-gralli), Brome grass (Bromus spp.), Barley grass (Hordeum spp. such as Hordeum glaucum and Hordeum leporinum), wild oat (Avena sp. such as Avena fatua), green foxtail (Setaria viridis), wild mustard (Sinapis arvensis), cleavers (Galium spurium and G. aparine), wild buckwheat (Polygonum convolvulus), kochia (Kochia scoparia), Jerusalem thorn (Parkinsonia aculeata), Palmer amaranth (Amaranthus palmeri), common lambsquarters (Chenopodium album), Wild radish (Raphanus raphanistrum), Wild cucurbits (Cucumis spp.), Creeping cucumber (Melothria pendula), Balsam apple or balsam pear (Momordica charantia), Kudzu (Pueraria montana var. lobata), Florida beggarweed (Desmodium tortuosum), clover (Trifolium spp.), Johnsongrass (Sorghum halepense), Jimsonweed (Datura stramonium), Fleabane (Conyza spp.), Common sowthistle (Sonchus oleraceus), Prickly lettuce (Lactuca serriola), Indian hedge mustard (Sisymbrium orientale) and Wild turnip (Brassica tournefortii). Annual ryegrass (Lolium rigidum) is the most important and costly weed to Australian winter crops with an estimated yield loss of $34.1 million to the Southern region. Wild oats (Avena spp.) is the most important winter cropping weed in northern New South Wales and southern Queensland. It is second in importance to annual ryegrass in most of the southern region and a significant weed in much of Western Australia. Kudzu (Pueraria montana var. lobata) is a notoriously noxious weed known in North America for its rapid growth rate and ability to smother native vegetation.

Chemical herbicides target essential processes and proteins in plants that are weeds for their inhibitory, even lethal, herbicide activity. The RNA molecules of the invention can be used to target the same processes and proteins. These processes include amino acid biosynthesis, photosynthesis, fatty acid synthesis, cell membrane integrity, pigment synthesis and plant development. Plant genes encoding proteins that are inactivated by chemical herbicides are well known. Consequently, the modes of action of chemical herbicides are classified into a number of groups including the following. Inhibitors of ACCase are in Group 1 and are used primarily for control of grasses. As a result, they are used primarily in broadleaf crops or fallow situations. Inhibitors of acetolactate synthase (ALS) are in Group 2. They inhibit ALS, required for branched chain amino acid synthesis, including for example the imidazolinones and sulfonylureas. Inhibitors of root growth are in Group 3, acting by stopping root extension. Group 4 are the synthetic auxins that act as growth regulators to disrupt plant growth and are generally used on monocotyledonous crops to control broadleaf weeds. Photosynthesis inhibitors are included in Groups 5, 6 and 7. These inhibit Photosystem II, examples include triazine and metribuzin. Shoot growth inhibitors are in Groups 8 and 15 and are soil-applied herbicides, often used to control weeds that have not emerged from the soil surface. Aromatic amino acid inhibitors are in Group 9, mainly glyphosate which is a non-selective herbicide that inhibits EPSP synthase (EPSPS). Glyphosate can be formulated as an ammonium, diammonium, dimethylammonium, isopropylamine or potassium salt. Glutamine synthesis inhibitors are in Group 10, for example glufosinate. Pigment synthesis inhibitors are in Groups 12, 13 and 27, including HPPD inhibitors such as mesotrione and isoxaflutole. PPO inhibitors are in Groups 14, acting as cell membrane disruptors. Inhibitors of Photosystem I are in Group 22, including chemicals such as paraquat and diquat and are used for non-selective weed control and crop desiccation prior to harvest. These herbicides are also referred to as cell membrane disruptors because of their contact activity. Each of the genes encoding these herbicide-targeted proteins can be used as a target gene for the RNA molecules of the inventions, especially the ledRNA molecules having the Δ22 modification as described herein because of their systemic and transitive amplification effects. However, the present invention is not limited to gene products that are inhibited by chemical herbicides; any essential gene in a weed species is a likely target gene. Even a family of target genes where there is redundancy of activity can be targeted by the RNA molecules of the invention, for example, by targeting conserved regions within the gene family. The compositions of the invention comprising multiple precursor RNA molecules targeting different target RNA molecules are preferred, i.e. providing for multiple modes of action.

In a preferred embodiment, the plant is an angiosperm.

In an embodiment, the plant is an oilseed plant, preferably an oilseed crop plant. As used herein, an “oilseed plant” is a plant species used for the commercial production of lipid from the seeds of the plant. The oilseed plant may be, for example, oil-seed rape (such as canola), maize, sunflower, safflower, soybean, sorghum, flax (linseed) or sugar beet. Furthermore, the oilseed plant may be other Brassicas, cotton, peanut, poppy, rutabaga, mustard, castor bean, sesame, safflower, Jatropha curcas or nut producing plants. The plant may produce high levels of lipid in its fruit such as olive, oil palm or coconut. Horticultural plants to which the present invention may be applied are lettuce, endive, or vegetable Brassicas including cabbage, broccoli, or cauliflower. The present invention may be applied in tobacco, cucurbits, carrot, strawberry, tomato, or pepper.

In a preferred embodiment, a transgenic plant of the invention is homozygous for each and every genetic modification that has been introduced (transgene) so that its progeny do not segregate for the desired phenotype. The transgenic plant may also be heterozygous for the introduced genetic modification/transgene(s), preferably uniformly heterozygous for the transgene such as for example, in F1 progeny which have been grown from hybrid seed. Such plants may provide advantages such as hybrid vigour, well known in the art.

### Genetic Modifications

Polynucleotides encoding precursor RNA molecules disclosed herein may be stably introduced to above referenced host cells and/or non-human organisms such as plants. For the avoidance of doubt, an example of the present disclosure encompasses an above referenced plant stably transformed with a polynucleotide encoding a precursor RNA molecule disclosed herein. As used herein, the terms “stably transforming”, “stably transformed” and variations thereof refer to the integration of the nucleic acid encoding the precursor RNA molecule into the genome of the cell such that they are transferred to progeny cells during cell division without the need for positively selecting for their presence. Stable transformants, or progeny thereof, can be identified by any means known in the art such as Southern blots on chromosomal DNA or PCR enabling their selection.

Transgenic plants can be produced using techniques known in the art, such as those generally described in Slater et al., Plant Biotechnology—The Genetic Manipulation of Plants, Oxford University Press (2003), and Christou and Klee, Handbook of Plant Biotechnology, John Wiley and Sons (2004).

In an embodiment, plants may be treated by topically applying an RNA molecule according to the present disclosure to the plant or a part thereof, or applying a polynucleotide encoding the RNA molecule. For example, the RNA molecule, preferably having a ledRNA structure, may be provided as a formulation with a suitable carrier and sprayed, dusted or otherwise applied to the surface of a plant or part thereof. The RNA molecule may be encompassed in a microbial cell, for example a yeast cell, in which it was produced, whereupon the RNA molecule is released to contact the plant cell or enters the plant cell, or the microbial cell comes into contact with a pest or pathogen, for example is ingested by an insect pest, where the RNA molecule reduces the amount or activity of a target RNA molecule. Accordingly, in an example, the methods of the present disclosure encompass introducing an RNA molecule disclosed herein to a plant, or a pest, or a pathogen thereof, the method comprising topically applying a composition comprising the RNA molecule to the plant or a part thereof.

Agrobacterium-mediated transfer is a widely applicable system for introducing genes into plant cells because DNA can be introduced into cells in whole plant tissues, plant organs, or explants in tissue culture, for either transient expression, or for stable integration of the DNA in the plant cell genome. For example, floral-dip (in planta) methods may be used. The use of Agrobacterium-mediated plant integrating vectors to introduce DNA into plant cells is well known in the art. The region of DNA to be transferred is defined by the border sequences, and the intervening DNA (T-DNA) is usually inserted into the plant genome. It is the method of choice because of the facile and defined nature of the gene transfer.

Acceleration methods that may be used include for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells, for example of immature embryos, by a propelling force. Exemplary particles include those comprised of tungsten, gold, platinum, and the like.

In another embodiment, gene editing is used to produce the genetically modified cell or organism such as, for example, a plant, to integrate a polynucleotide of the invention into the genome of the cell or organism. Briefly, endonucleases can be used to generate single stranded or double-stranded breaks in genomic DNA. The genomic DNA breaks in eukaryotic cells are repaired using non-homologous end joining (NHEJ) or homology directed repair (HDR) pathways, for example using a template DNA comprising a polynucleotide of the invention. NHEJ may result in imperfect repair resulting in unwanted mutations and HDR can enable precise gene insertion by using an exogenous supplied repair DNA template. CRISPR-associated (Cas) proteins have received significant interest although transcription activator-like effector nucleases (TALENs) and zinc-finger nucleases are still useful, the CRISPR-Cas system offers a simpler, versatile and cheaper tool for genome modification.

In another method, plastids can be stably transformed. Methods disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265). Other methods of cell transformation can also be used and include but are not limited to the introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., In: Methods for Plant Molecular Biology, Academic Press, San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polynucleotide is cultivated using methods well known to one skilled in the art.

To confirm the presence of a genetic modification, a polymerase chain reaction (PCR) amplification or Southern blot analysis can be performed using methods known to those skilled in the art. Expression products of the genetic modification can be detected in any of a variety of ways, depending upon the nature of the product, and include Northern blot hybridisation, Western blot and enzyme assay. Once genetically modified plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

Genetically modified plants formed using Agrobacterium or other transformation methods typically contain a single genetic locus on one chromosome. Such genetically modified plants can be referred to as being hemizygous for the genetic modification. More preferred is a genetically modified plant that is homozygous for the genetic modification, that is, a genetically modified plant that contains two added genetic modifications, one genetic modification at the same locus on each chromosome of a chromosome pair. A homozygous transgenic plant can be obtained by self-fertilising a hemizygous genetically modified plant, germinating some of the seed produced and analysing the resulting plants for the genetic modification of interest.

It is also to be understood that two different transgenic plants that contain two independently segregating genetic modifications or loci can also be crossed (mated) to produce offspring that contain both sets of genetic modifications or loci. Selfing of appropriate F1 progeny can produce plants that are homozygous for both genetic modifications or loci. Back-crossing to a parental plant and out-crossing with a non-genetically modified plant are also contemplated, as is vegetative propagation. Similarly, a genetically modified plant can be crossed with a second plant comprising a genetic modification such as a mutant gene and progeny containing both of the transgene and the genetic modification identified. Descriptions of other breeding methods that are commonly used for different traits and crops can be found in Fehr, In: Breeding Methods for Cultivar Development, Wilcox J. ed., American Society of Agronomy, Madison Wis. (1987).

### Target RNA

The RNA molecules of the present disclosure can decrease expression or amount of various target RNA molecules, thereby decreasing the activity of the target gene.

In an embodiment, the target RNA molecule is a fatty acid biosynthesis gene. Examples of such genes include genes encoding acetyl transacylases, acyl transport proteins (“acyl carrier protein”), desaturases such as stearyl desaturases or microsomal D12-desaturases, in particular Fad2-1 genes, malonyl transacylase, 3-ketoacyl-ACP synthetases, 3-keto-ACP reductases, enoyl-ACP hydrases, thioesterases such as acyl-ACP thioesterases, enoyl-ACP reductases. In an embodiment, the target RNA molecule is FAD2 gene (for example those described by Genbank Acc. No.: AF124360 (Brassica carinata), AF042841 (Brassica rapa), L26296 (Arabidopsis thaliana), A65102 (Corylus avellana)). For example, the target RNA molecule can be FAD2.1 or FAD2.2 gene.

Examples of other genes involved in modifying lipid composition that encode a target RNA molecule are known in the art (see, for example, Shure et al., 1983; Preiss et al., 1987; Gupta et al., 1988; Olive et al., 1989; Bhattacharya et al., 1990; Dunwell, 2000; Brar et al., 1996; Kishore and Somerville, 1993; U.S. Pat. No. 5,530,192 and WO 94/18337).

In another embodiment, the target RNA molecule is an arthropod gene such as an insect, arachnid, or decapod gene transcript. Examples of such genes include chitin synthase genes, such as CHS1 and/or CHS2 or other genes that control insect activity, behaviour, reproduction, growth and/or development. Exemplary target RNA molecules in insect pests such as Helicoverpa armigera or Spodoptera are described herein. Various essential genes of a variety of pathogens are known to the those of skill in the art, for example nematode resistance genes are summarised in WO 93/10251, WO 94/17194.

In another embodiment, the target RNA molecule is associated with delay of fruit maturation. Delayed fruit maturation can be achieved for example by reducing the gene expression of genes selected from the group consisting of polygalacturonases, pectin esterases, β-(1-4) glucanases (cellulases), β-galactanases (β-galactosidases), or genes of ethylene biosynthesis, such as 1-aminocyclopropane-1-carboxylate synthase, genes of carotenoid biosynthesis such as, for example, genes of prephytoene or phytoene biosynthesis, for example phytoene desaturases.

In another embodiment, the target RNA molecule is associated with delay of senescence symptoms. Suitable target RNA molecules include cinnamoyl-CoA:NADPH reductases or cinnamoyl alcohol dehydrogenases. Further target RNA molecules are described in WO 1995/07993.

In another embodiment, the target RNA molecule is associated with modification of the fibre content in foodstuffs, preferably in seeds. For example, the RNA molecule can reduce expression of caffeic acid O-methyltransferase or of cinnamoyl alcohol dehydrogenase.

In an embodiment, the plant phenotype conferred by means of the reduced target RNA molecule is selected from: improved abiotic stress tolerance, improved biotic stress tolerance, improved resistance to a pest or pathogen of said plant, for example a plant-pathogenic fungus or a plant virus, modified primary metabolite composition, modified secondary metabolite composition, modified trace element, carotenoid, or vitamin composition, improved yield, improved ability to use nitrogen or other nutrients, modified agronomic characteristics, modified growth or reproductive characteristics, and improved harvest, storage, or processing quality.

Plant viruses of economic importance have been reviewed by Tatineni and Hein (2023). These include Banana bunchy top virus (genus Babuvirus) in banana, Cassava mosaic virus in cassava (Manihot esculenta), Citrus tristeza virus (genus Closterovirus) in citrus, Grapevine leafroll-associated virus in grapevine (Vitis spp.), Maize dwarf mosaic virus (genus Potyvirus) in maize (Zea mays), Maize streak virus (genus Mastrevirus) in the family Poaceae, Potato virus Y (genus Potyvirus) in potato (S. tuberosum), Rice tungro spherical virus and rice tungro bacilliform virus in rice, Rice yellow mottle virus (genus Sobemovirus) in rice, Plum pox virus (genus Potyvirus) in stone fruits (Prunus spp.), Sweet potato chlorotic stunt virus and sweet potato feathery mottle virus in sweet potato (Ipomoea batatas), Cucumber mosaic virus (genus Cucumovirus) in vegetables, ornamentals and woody plants, Tomato spotted wilt virus (genus Orthotospovirus) infecting more than 800 species, especially plants in the Solanaceae and Compositae, Tomato yellow leaf curl virus (genus Begomovirus) in tomato (Solanum lycopersicum), Barley yellow dwarf virus (genus Luteovirus) and cereal yellow dwarf virus (genus Polerovirus) in wheat, barley, oats and maize, Cassava brown streak virus in cassava, Zucchini yellow mosaic virus (genus Potyvirus) in Cucurbits, Faba bean necrotic yellows virus (genus Nanovirus) in faba bean (Vicia faba), associated virus (genus Grablovirus) in grapevine, Papaya ringspot virus (genus Potyvirus) in papaya (Carica papaya), Groundnut bud necrosis virus (genus Orthotospovirus) in peanut (Arachis hypogaea), Pepino mosaic virus (genus Potexvirus) in tomato, Tomato brown rugose fruit virus (genus Tobamovirus) in tomato and capsicum, Cucumber green mottle mosaic virus (genus Tobamovirus) in vegetables and Cucurbits, and Wheat streak mosaic virus (genus Tritimovirus) in wheat and barley. The RNA molecules of the present invention, especially the asymmetric RNA molecules having the Δ22 modification, can be used to down-regulate the genomic RNAs or RNA transcripts from these viruses, for example to control these plant viruses or reduce symptoms of infection, preferably done by expressing the RNA molecules from a polynucleotide in a transgenic plant of a species that is infected by the specific virus.

The use of dsRNA molecules to provide resistance to plant viruses has been reviewed by Cisneros and Carbonell (2020), Gaffar and Koch (2019), Kim et al., (2019), Ricci et al., (2021), Robinson et al., (2014) and Rosa et al., (2018). The references listed in those reviews, for example Table 1 of Gaffar and Koch (2019) and Table 1 of Cisneros and Carbonell (2020), describe many plant viral genes that can be targeted by RNAi. The RNA molecules of the present invention directed to those genes provide increased control of viral infection.

### Methods of Controlling Non-Human Organisms

The RNA molecules according to the present invention can be used to control non-human organisms such as fungi, insects, arachnids or nematodes that are pests or pathogens. Such uses involve administering RNA molecules according to the present disclosure using various methods. In an embodiment, the present disclosure encompasses methods of controlling an insect, the method comprising spraying, dusting or otherwise applying RNA molecules, or cells such as microbial cells producing the RNA molecules of the invention disclosed herein, to the insects. In this embodiment, the RNA molecules or the microbial cells can be sprayed, dusted or otherwise applied directly to the insects. In an embodiment, the RNA molecules or the microbial cells can be sprayed, dusted or otherwise applied directly to plant tissues such as leaves, stems or reproductive tissues so that they are ingested by the insect. The insects are preferably larvae. In another embodiment, the RNA molecules can be sprayed, dusted or otherwise applied to plants or crops prior to insect infestation. In an embodiment, RNA molecules of the invention can be provided for ingestion by insects, for example incorporated into a bait. The compositions and RNA molecules disclosed herein may be provided in a dispenser. In an embodiment, the dispenser is a trap or a lure. In an embodiment, the trap and/or lure comprises a bait comprising an RNA molecule(s) disclosed herein. In another embodiment, the RNA molecules can be sprayed onto fungi or on to plant tissues which are susceptible to fungi, for example leaves, as required. The RNA molecules may be applied in a seed coating to seeds, prior to sowing. Exemplary plants include cotton, maize, wheat, barley, soybean, canola, tomato, chickpea, pigeon pea, alfalfa, rice, sorghum and cowpea.

The RNA molecules according to the present invention can be provided to modify insect behaviour. For example, the RNA molecules can be provided to reduce insect fertility or modify feeding behaviour. In an embodiment, the target gene(s) is essential for the insect or at least important to its growth, development, fertility, reproduction or survival, such that silencing that gene(s) causes lethality, developmental slowing or arrest, infertility or reduced fecundity. In an embodiment, the insect is contacted with the RNA molecules at the right developmental stage and in sufficient amount.

In another embodiment, the RNA molecules can be provided to kill insects or nematodes. The present invention therefore provides a dead insect or nematode comprising one or more RNA molecules of the invention.

Target insects that can be controlled by the RNA molecules of the invention include household insects, ecto-parasites and insects and/or arachnids relevant for public health and hygiene such as, by way of example and not limitation, flies, spider mites, thrips, ticks, red poultry mite, ants, cockroaches, termites, crickets including house-crickets, silverfish, booklice, beetles, earwigs, mosquitoes and fleas. Other exemplary target insects include agricultural pests such as sap feeders, for example stink bugs and aphids, chewing insects such as caterpillars, beetles, rasping insects such as thrips and slugs, moths, fruit fly, and grain pests such as grain borer, weevils and grain moths. Examples of target aphids are the species Myzus persicae, Metopolophium dirhodum, Rhopalosiphum padi, Aphis glycines and Aphis fabae.

In an embodiment of the invention, the insect or arachnid belongs to one of the following Orders: Acari, Arachnida, Anoplura, Blattodea, Coleoptera, Collembola, Dermaptera, Dictyoptera, Diplura, Diptera, Embioptera, Ephemeroptera, Grylloblatodea, Hemiptera, Heteroptera, Homoptera, Hymenoptera, Isoptera, Lepidoptera, Mallophaga, Mecoptera, Neuroptera, Odonata, Orthoptera, Phasmida, Phithiraptera, Plecoptera, Protura, Psocoptera, Siphonaptera, Siphunculata, Thysanura, Sternorrhyncha, Strepsiptera, Thysanoptera, Trichoptera, Zoraptera and Zygentoma. In preferred, but non-limiting, embodiments of the invention the insect or arachnid is chosen from the group consisting of: (1) Acari: mites including Ixodida (ticks), (2) Arachnida: Araneae (spiders) and Opiliones (harvestman), examples include: Latrodectus mactans (black widow) and Loxosceles recluse (Brown Recluse Spider), (3) Anoplura: lice, such as Pediculus humanus (human body louse), (4) Blattodea: cockroaches such as German cockroach (Blatella germanica), of the genus Periplaneta, such as American cockroach (Periplaneta americana) and Australian cockroach (Periplaneta australiasiae), of the genus Blatta, including Oriental cockroach (Blatta orientalis) and of the genus Supella, including brown-banded cockroach (Supella longipalpa), (5) Coleoptera: beetles, examples include Leptinotarsa sp. such as Leptinotarsa decemlineata (Colorado Potato Beetle), Diabrotica sp. such as Diabrotica virgifera (Corn rootworm), Powderpost beetle (family of Bostrichoidea), Dendroctonus spp. (Black Turpentine Beetle, Southern Pine Beetle, IPS Engraver Beetle), Carpet Beetles (Anthrenus spp, Attagenus spp), Old House Borer (family of Cerambycidae: Hylotrupes bajulus), Anobium punctatum, Tribolium spp (flour beetle), Trogoderma granarium (Khapra Beetle), Oryzaephilus sarinamensis (Toothed Grain Beetle) etc, (6) Dermaptera: family of earwigs, (7) Diptera such as mosquitoes (Culicidae) and flies (Brachycera), examples include: Anophelinae such as Anopheles spp. and Culicinae such as Aedes fulvus, Tabanidae such as Tabanus punctifer (Horse Fly), Glossina morsitans morsitans (tsetse fly), drain flies (Psychodidae) and Calyptratae such as Musca domestica (House fly), flesh flies (family of Sarcophagidae), (8) Heteroptera: bugs, such as Cimex lectularius (bed bug), (9) Hymenoptera: wasps (Apocrita), including ants (Formicoidea), bees (Apoidea) such as Solenopsis invicta (Red Fire Ant), Monomorium pharaonis (Pharaoh Ant), Camponotus spp (Carpenter Ants), Iasius niger (Small Black Ant), Tetramorium caespitum (Pavement Ant), Myrmica rubra (Red Ant), Formica spp. (wood ants), Crematogaster lineolata (Acrobat Ant), Iridomyrmex humilis (Argentine Ant), Pheidole spp. (Big Headed Ants, Dasymutilla occidentalis (Velvet Ant), (10) Isoptera: termites, examples include: Amitermes floridensis (Florida dark-winged subterranean termite), eastern subterranean termite (Reticulitermes flavipes), R. hesperus (Western Subterranean Termite), Coptotermes formosanus (Formosan Subterranean Termite), Incisitermes minor (Western Drywood Termite), Neotermes connexus (Forest Tree Termite) and Termitidae, (11) Lepidoptera: moths, examples include: Helicoverpa sp. such as Helicoverpa armigera and Helicoverpa zea, Spodoptera sp. such as Spodoptera frugiperda, Plutella sp. such as Plutella xylostella (Diamond backed moth), Tineidae & Oecophoridae such as Tineola bisselliella (Common Clothes Moth), and Pyralidae such as Pyralis farinalis (Meal Moth), (12) Psocoptera: booklice (Psocids), (13) Siphonaptera: fleas such as Pulex irritans, (14) Sternorrhyncha: aphids (Aphididae), and (15) Zygentoma: silverfish, examples are: Thermobia domestica and Lepisma saccharina.

The European Commission has listed the 20 invasive pests and pathogen species of concern for agriculture, as follows: Agrilus anxius (bronze birch borer), Agrilus planipennis (emerald ash borer), Anastrepha ludens (Mexican fruit fly), Anoplophora chinensis (citrus long-horned beetle), Anoplophora glabripennis (Asian long-horned beetle), Anthonomus eugenii (pepper weevil), Aromia bungii (Red-necked longhorn beetle), Bactericera cockerelli (potato psyllid), Bactrocera dorsalis (oriental fruit fly), Bactrocera zonata (peach fruit fly), Bursaphelenchus xylophilus (pine wood nematode or pine wilt nematode), Candidatus Liberibacter spp., (causal agent of Huanglongbing disease of citrus/citrus greening), Conotrachelus nenuphar (plum curculio), Dendrolimus sibiricus (Siberian silk moth), Phyllosticta citricarpa (fungus causing citrus black spot), Popillia japonica (Japanese beetle), Rhagoletis pomonella (apple maggot), Spodoptera frugiperda and Thaumatotibia leucotreta (false codling moth). All of these insect pests and fungal pathogens can be controlled using the RNA molecules of the present invention.

In an embodiment the insect is a sap sucking insect. For example, the RNA molecule can reduce activity of gene(s) encoding MpC002 and/or MpRack-1.

Alternatively, the target gene is other than a gene encoding MpC002 and/or MpRack-1 in Myzus persicae. In an embodiment the sap sucking insect is an aphid such as, for example, Myzus persicae.

In an embodiment the insect target is an ant (e.g. Linepithema humile), cotton bollworm (Helicoverpa armigera), Pectinophora gossypiella (Pink bollworm), or blowfly (e.g. Lucilia cuprina). Alternatively, the insect is other than H. armigera, for example a Lepidopteran insect other than H. armigera. In an embodiment the target insect is Helicoverpa armigera and the target RNA molecule is not a transcript from the ABC transporter white gene (ABC white, Accession No. KU754476) or a gene encoding a pheromone biosynthesis activating neuropeptide (PBAN) in H. armigera. In another embodiment the target insect is Linepithema humile and the target RNA molecule is not a transcript from a gene encoding PBAN (Accession No. XM_012368710). Alternatively, the insect is other than L. humile, for example a Hymenopteran insect other than L. humile. In another embodiment the target insect is Lucilia cuprina and the target RNA molecule is not a transcript from a gene encoding a protein selected from the group consisting of V-type proton ATPase catalytic subunit A, RNAse 1/2, chitin synthase, ecdysone receptor and gamma-tubulin 1/1-like. Alternatively, the insect is other than Lucilia cuprina, for example a Dipteran insect other than Lucilia cuprina.

In an embodiment, the target RNA is an insect gene transcript or a homolog of one of the target RNA molecules listed in Example 8 herein. These are further described below. In an embodiment, the insect is wild-type for Dicer i.e. is not modified in its Dicer.

### Difficulties Previously Reported for Use of Conventional RNAi in Insects

Numerous general reviews have summarised attempts reported in the literature to silence genes by use of dsRNA molecules in insects (Burand and Hunter, 2013; Christiaens et al., 2020; Chung et al., 2021; Fernandez et al., 2021; Kaur et al., 2021; Kunte et al., 2019; Li et al., 2022; Silver et al., 2021; Velez et al., 2023; Zhang et al., 2017; Zhu and Palli, 2020). Other reviews focus on the use of conventional dsRNA molecules to reduce gene activity more specifically in Lepidopteran or Coleopteran insects, including Helicoverpa and Spodoptera species (Baum and Roberts, 2014; Kolliopoulou and Swevers 2014; Lucena-Leandro et al., 2022; Nitnavare et al., 2021; Terenius et al., 2011; Xu et al, 2016). Many of the reviews describe various methods to delivering the conventional dsRNA molecules into the insect cells, for example the use of nanoparticles, liposomes or bacterial encapsulation as protective means to enable delivery (Adeyinka et al., 2020; Chung et al., 2021; Cooper et al., 2018; Joga et al., 2016; Lucena-Leandro et al, 2022). Oral delivery or injection of dsRNA into whole insects have been used to identify target genes in an insect (Mehlhorn et al., 2021). Some of these reviews also describe the possibility of expressing dsRNA molecules in transgenic plants, to be ingested by the insects for silencing the target gene(s) (Chung et al., 2021; Li et al., 2022). Zhang et al. (2017) concluded that practical application of this strategy was challenging because of the difficulty in producing sufficient amounts of stable dsRNA such as conventional hairpin RNA molecules by in planta expression from transgenes in plants.

Many of the reviews refer to the difficulties of achieving effective control of insect pests with conventional RNAi, particularly for oral delivery of the RNA molecules (Huvenne and Smagghe, 2010). As the functional core of the alimentary canal, the midgut of insects is responsible for food digestion and nutrient absorption, which is pivotal for many physiological processes such as growth and development, energy storage and reproduction (Holtof et al., 2019). Many insects respond to conventional orally-delivered dsRNA molecules by degrading the molecules in the gut before they can cross the gut wall, or the dsRNA molecules are degraded in the hemolymph, or they are not released from endosomes and therefore do not enter the cytoplasm in the target cells and are therefore unavailable for RNAi (Wang et al., 2016). Efficient silencing by dsRNA by feeding was enhanced when larvae were used immediately after molting and starvation for 24 h (Rodríguez-Cabrera et al., 2010), suggesting that degradation of dsRNA in the midgut was decreased due to starvation and could be an important factor for the increased sensitivity to dsRNA.

Despite numerous reports of attempts to demonstrate RNAi in lepidopterans, insects of the order Lepidoptera appear to be more refractory than other insect orders such as Coleoptera (Terenius et al., 2011). RNA degradation in the lepidopteran larval gut is aided by the alkaline conditions of the gut contents in addition to the RNAses secreted into the gut lumen that are specific for dsRNA molecules (dsRNAse) (Cooper et al., 2018; Cooper et al., 2020; Guan et al., 2018; Guan et al., 2019; Peng et al., 2020a; Peng et al., 2020b; Reis et al., 2022; Yao et al., 2022). Several other factors may play a role in lepidoterans, for example lack of certain components of the RNAi machinery in these insects such as SID1, insufficient dose or concentration of the dsRNA or the insect life stage and tissue used, and these factors may limit the efficiency of insecticidal RNAi (Guan et al., 2018; Peng et al., 2019; Rodríguez-de la Noval et al., 2019; Liu et al., 2020; Chen et al., 2021).

The target genes described or reported in all of the above references may be targeted with the RNA molecules of the present invention, including in conjunction with the delivery methods known in the art, including those described herein.

### Helicoverpa Species

Several Helicoverpa species, including H. armigera (cotton bollworm, also known as cotton budworm), H. punctigera (Australian bollworm), Pectinophora gossypiella (Pink bollworm), H. assulta (oriental tobacco budworm) and H. zea (corn earworm), and related Heliothis species such as Heliothis virescens (tobacco budworm) are damaging agricultural pests and can be controlled with the RNA molecules of the present invention, particularly those having G:U basepairs, such as hairpin RNAs having G:U basepairs, as described herein. Helicoverpa species are major insect pests of important crops such as cotton, corn, soybean, chickpeas, tomatoes, potatoes and leafy vegetables.

According to reports in the literature, a wide variety of target genes in H. armigera have been tested for suppression with conventional dsRNA by oral delivery to larvae, with highly variable results that are often transient and require large quantities of dsRNA to be delivered, reviewed by Lim et al. (2016). The factors that contribute to the variability include larval gut conditions which quickly degrade dsRNA in the digestive system, in particular the alkaline pH of the midgut contents and presence of RNases. Other factors include an apparent lack of a substantial RNAi amplification mechanism in H. armigera, evidenced by an absence of a gene for RNA-dependent RNA polymerase (RdRp) in the insect genome, target genes not being expressed in midgut cells, insufficient expression of SID-1 and SID-2 genes which are responsible for dsRNA uptake and transport, and potential blocks to the processing and release of RNA from vesicles after being taken up by endocytosis. Lim et al. (2016) recommended targeting genes which are expressed in the midgut with lower transcript abundance.

Despite those difficulties, there are various reports of target genes that were successfully down-regulated by oral delivery of conventional dsRNA to H. armigera larvae, including target genes encoding acetylcholinesterase (AChE, Bally et al., 2016; Kumar et al., 2009), adenylate kinase 2 (Chen et al., 2012), arginine kinase (Ai et al., 2019; Liu et al., 2015; Qi et al., 2015), chitinase (Mamta et al., 2016; Yang and Han, 2014b), chitin synthase (Jin et al., 2015), coatomer beta (Mao et al., 2015), cytochrome P450s such as CYP6B6 (Zhang et al., 2013; Zhao et al., 2016) or CYP6AE14 (Mao et al., 2007), ecdysone receptor (EcR, Yang and Han, 2014a; Zhu et al., 2012), ecdysone receptor and insect intestinal mucin (IIM) Israni and Rajam (2016), glutathione-S-transferase (GST, Mao et al., 2007; Shabab et al., 2014), HMG-COA reductase (HMGR, Tian et al., 2015), HR3 molt-regulating transcription factor (Han et al., 2017; Xiong et al., 2013), neuropeptide F (NPF, Yue et al., 2016), transferrin (Zhang et al., 2015b), trypsin protease (Chandra et al., 2018) and v-ATPase (Fu et al., 2020; Mao et al., 2015; Jin et al., 2015). Asokan et al. (2013 and 2014) targeted several different genes including those encoding chymotrypsin, GST, JAHMT, cytochrome P450 and trypsin through use of dsRNAs of 93-470 basepairs delivered in a semi-synthetic diet, so testing detoxification genes, proteases involved in digestion of proteins and a gene involved in larval metamorphosis. Also using oral delivery with dsRNA-containing diets, Chikate et al. (2016) targeted various enzymes and proteins including the proteases trypsin (HaTry2, 3, 4 and 6), chymotrypsin (HaChy4) and cysteine protease like cathepsin (HaCATHL), as well as GSTs (HaGST1a, 6 and 8), esterases (HaAce4, HaJHE), catalase (HaCAT), super-oxide-dismutase (HaCu/ZnSOD), fatty acid binding protein (HaFabp) and chitin deacetylase (HaCda5b). They observed reduction in the amount of target transcripts. Vatanparast et al. (2021) also tested dsRNAs targeting protease gene transcripts. Jaiwal et al. (2020) evaluated dsRNA targeting six hormonal biosynthesis genes of H. armigera, namely juvenile hormone acid methyltransferase (JHAMT), prothoracicotropic hormone (PTTH), pheromone biosynthesis-activating peptide (PBAP), molt-regulating transcription factor (HR3), activated protein 4 (AP-4) and eclosion hormone precursor (EHP) and observed 60-90% mortality of larvae. Oral delivery using transgenic plant material expressing the dsRNA targeting AChE was shown by Bally et al., (2016) using chloroplast transformation. Fu et al. (2020) similarly used chloroplast transformation of tobacco to express a long dsRNA targeting v-ATPase but did not observe increased mortality of H. armigera larvae. Nuclear transformants did cause delayed larval growth. They suggested that plant-derived siRNAs were more effective triggers of RNAi in Lepidoptera than longer dsRNAs.

Oral delivery of conventional dsRNA when expressed in transgenic plants has also been demonstrated with H. armigera. Han et al. (2017) produced transgenic cotton plants expressing a conventional hairpin RNA targeting the HR3 gene (Accession No. FJ009448) and observed larval mortality when the plant leaves were fed to H. armigera larvae. Liu et al. (2015) produced transgenic Arabidopsis thaliana plants expressing a hairpin RNA against the arginine kinase (AK) gene transcript and observed mortality when H. armigera larvae were fed on the transgenic leaves. Mamta et al. (2016) produced transgenic tobacco and tomato plants expressing a hairpin RNA against a chitinase gene transcript and observed delayed growth and some mortality when H. armigera larvae were fed on the transgenic leaves. Mao et al. (2007) produced transgenic tobacco and A. thaliana plants expressing a hairpin RNA against a cytochrome P450 gene (CYP6AE14) transcript and observed delayed growth and some mortality when H. armigera larvae were fed on the transgenic leaves. This effect was also observed in transgenic cotton (Mao et al., 2011). Tian et al. (2015) produced transgenic cotton plants expressing a conventional hairpin RNA targeting the HMGR gene (Accession No. GU584103) and observed delayed larval growth when fed the plant leaves. Wu et al. (2016) produced transgenic cotton plants expressing a conventional hairpin RNA targeting a gene from mitochondrial complex I, a NADH: ubiquinone oxidoreductase (NDUFV2, Accession No. KR152651) and observed larval mortality up to 80% when fed the plant leaves.

The RNA molecules of the present invention can be used to down-regulate the target RNA transcripts of all of the above-mentioned genes in Helicoverpa and Heliothis species, including Helicoverpa armigera, particularly for the hairpin RNA or ledRNA molecules comprising G:U basepairs in the dsRNA region when expressed in planta from a transgene, preferably with 15-30% G:U basepairs across dsRNA regions of at least 100 basepairs in length, or without G:U basepairs when the RNA molecules are applied topically or expressed in yeast, with increased effectiveness compared to the corresponding conventional RNA molecule. The asymmetric hairpin and ledRNA molecules, particularly having the Δ22 modification across dsRNA regions of at least 100 basepairs in length, are also effective, without, or preferably with, the G:U basepairs. The insect pests are thereby controlled for plants such as cotton plants (Gossypium hirsutum and G. barbadense).

Spodoptera frugiperda and Other Spodoptera Species

The fall armyworm (FAW) Spodoptera frugiperda (Lepidoptera) is one of the most destructive insect pests and significantly constrains crop production worldwide, resulting in severe losses to maize, rice, wheat, sorghum, cotton, soybean and other crops, particularly in North and South America, Asia and Africa. Yield losses by FAW can be 30%-60% of the crop. S. frugiperda has a wide host range, the larvae feeding on at least 350 different plant species from more than 70 plant families (Liu et al., 2020b; Overton et al., 2021). The management of S. frugiperda is challenging due to its biological characteristics and its ability to survive in various environments (Wan et al., 2021b). Synthetic insecticides are still the major approach for controlling insect pests such as S. frugiperda, but their use is associated with significant hazards to human health and the environment along with reduced efficacy due to the emergence of pest resistance to the insecticides.

As an invasive insect pest, S. frugiperda has a pronounced capacity to occupy wide ecological zones in a short period due to its high reproductive rate, migration capacity, polyphagous nature and extensive host range (Johnson, 1987; Du-Plessis et al., 2020). Its life cycle is relatively short, with stages for eggs (2-3 days), larvae (13-14 days), pupae (7-8 days) and adults (7-21 days), so having a generation time of about 40 days during warm summer months and about 55 days at cooler temperatures (Wan et al., 2021b). A single adult female can lay 1,500-2,000 eggs in her life, usually in batches of 100-200 eggs on plant leaves (Johnson, 1987). After hatching, neonates progress through six larval instars over 2-3 weeks, with the sixth instar being the most destructive in causing about 70% of S. frugiperda damage to plants. Two morphologically indistinguishable races of S. frugiperda have been identified, namely, the “corn” strains which prefer to feed mainly on corn (maize), sorghum and cotton, and the “rice” strains which prefer to feed mainly on rice, alfalfa, millet and pasture grasses (Dumas et al. 2015). Despite their morphological similarity, however, the two strains differ somewhat in genetics, physiology, and behavioural features, such as mating and insecticidal resistance.

As in many other lepidopteran insects, gene silencing in S. frugiperda larvae by oral delivery of dsRNA molecules has proven difficult to achieve to sufficient extent-indeed the present inventors are not aware of any reports of ingestion of transgenic plant tissues expressing a conventional dsRNA that was effective in reducing a target gene activity to provide a deleterious phenotype to the larvae or effective control of S. frugiperda. This is at least because of degradation of conventional dsRNA molecules in the S. frugiperda larval gut and poor transport of dsRNA from the gut contents across the gut wall and into cells or the hemolymph. In addition, S. frugiperda appears to lack intercellular transport of the dsRNA molecules or the 21/21-mer siRNAs produced from those molecules by Dicer, due to the absence of RdRp enzyme as identified in other Lepidopteran insects (Shukla et al., 2016). That is, these insects lack an efficient systemic RNAi when using conventional dsRNA molecules, which refers to the intercellular spreading of RNAi silencing throughout the larval body after dsRNA is delivered into a specific tissue or region. S. frugiperda lacks the transmembrane protein SID1, known to function in many organisms as a dsRNA-selective channel that enables bidirectional transport of dsRNA molecules between cells (Zhu and Palli, 2020).

Nevertheless, there are numerous reports of attempts to reduce specific gene activity with conventional RNAi molecules in S. frugiperda and other Spodoptera species, including by feeding from synthetic media. Some of these reports have been reviewed by Kebede and Fite (2022). Most of these studies targeted genes for insect growth, reproduction or survival, for example growth regulating hormones. Many of the reports listed in Table 1 of Kebede and Fite (2022) used injection of the dsRNA, not oral delivery, whereas others used feeding of bacteria producing the dsRNA or of nanoparticles to protect the dsRNA upon ingestion. None of those reports successfully used in planta expression of conventional dsRNA molecules with feeding to the insect larvae to control the pests.

An et al. (2023) fed S. frugiperda larvae with dsRNA molecules, produced in E. coli HT115 cells, for three days from a synthetic medium. They targeted a gene encoding a prohibitin 1 (PHB1) protein and reported significantly increased Vip3A virulence in the larvae. Bai et al. (2023) constructed baculovirus vectors expressing dsRNAs targeting a v-ATPase-A (Accession No. XP_022826560.1 in S. litura) gene and a v-ATPase-B (Accession No. XP_022827405.1 in S. litura) gene of S. frugiperda and, after injection into larvae, observed gene down-regulation. Griebler et al. (2008) injected S. frugiperda larva or adult females with a dsRNA targeting transcripts of genes encoding allatostatin or allatotropin proteins (Accession No. DQ208707 in Mythimna separata) and observed reduced transcript levels and some phenotypes in the larvae. Hafeez et al. (2022) and Hafeez et al. (2023) fed S. frugiperda larvae with dsRNA molecules in a synthetic delivery buffer, targeting genes encoding three cytochrome P450 detoxification proteins (CYP321A7, CYP6AE43, CYP302A1) and reported several resultant phenotypes. Zhang et al. (2020) carried out similar feeding experiments. Park and Vatanparast (2022) injected S. frugiperda larvae with a dsRNA targeting the gene encoding PBAN receptor (Accession No. GSE175545) and observed a reduction in fecundity. Feeding or injection of S. frugiperda larvae with dsRNA reduced the activity of a serine-protease gene (SfT6, Accession No. FJ940726) in the midgut tissue (Rodríguez-Cabrera et al. 2010). Rodriguez-de la Noval et al. (2019) fed S. frugiperda larvae with a 466 basepair dsRNA in a synthetic delivery buffer, targeting a gene encoding peritrophin A protein (SfPER Accession no. EL618681), and reported decreased pupal weight and adult emergence. Yoon et al. (2017) demonstrated a lack of processing of dsRNA into siRNAs due to entrapment of the dsRNAs in endosomes, or acidic bodies. There was no attempt to reduce gene activity in that report.

Other studies have been reported where different dsRNA delivery methods were tried with S. frugiperda larvae, including encapsulating the dsRNA molecules in nanoparticles (Dhandapani et al., 2022; Gurusamy et al., 2020a; McGraw et al., 2022) or formulated with transfection reagents in the form of liposomes (Gurusamy et al., 2020b), or complexing the dsRNA with cationic polymers (Christiaens et al., 2018; Parsons et al., 2018). In some cases, a reduction in target gene activity was observed with resultant phenotypes. Wan et al. (2021) fed S. frugiperda larvae on bacteria producing dsRNA molecules targeting four different genes (Sf-CHI, Sf-CHSB, Sf-ST and Sf-HEM), or injected the dsRNAs, and observed variable RNAi efficiency. A decrease in larval body weight was observed due to the knockdown of these genes. WO2021/231791 describes compositions and methods for controlling DBM or FAW, including numerous candidate target genes therein (SEQ ID NOs 115-1682 in WO2021/231791).

Similar attempts have been reported for other species of Spodoptera. For example, Vatanparast and Kim (2017) injected or fed larvae of the beet armyworm Spodoptera exigua with in vitro synthesised conventional dsRNA molecules. The dsRNA, produced in E. coli strain HT115, targeted a gene encoding a chymotrypsin. They reported that a large amount of dsRNA was required to kill late instar S. exigua because of high RNAse activity in the larval midgut lumen. Tian et al. (2009) similarly expressed a conventional dsRNA in E. coli HT115, targeting a gene encoding chitin synthase A (SeCHSA), and observed disturbed growth and development of the larvae after ingestion of the bacteria. To screen for effective target genes in S. exigua, Li et al. (2013) selected nine candidate genes and injected siRNA molecules targeting the gene transcripts. The mRNA for eight of the genes, namely genes encoding chitinase 7, plasma glutamate carboxypeptidase (PGCP), chitinase 1, ATPase, tubulin 1, ADP-ribosylation factor 2 (ARF 2), tubulin 2 and helicase decreased after injection. PGCP plays a key role in the metabolism of secreted peptides. Martinez et al. (2021) fed S. exigua larvae with leaves that had been coated with a mixture of a lectin-dsRNA binding domain (RBD) fusion protein and a dsRNA targeting a v-ATPase gene and observed increased mortality compared to the dsRNA alone. Tang et al. (2010b) targeted a gene encoding trehalose-6-phosphate synthase from Spodoptera exigua (SeTPS, Accession No. EF051258). Shi et al. (2018) targeted a gene encoding Rieske iron-sulfur protein (RISP, Accession No. JN992290) which is a key protein subunit of mitochondrial complex III and therefore plays an important role in the respiratory electron transport chain.

Lu et al. (2015) injected a dsRNA targeting PBAN into common cutworm moth (Spodoptera litura, Lepidoptera) larvae and observed reduced gene expression. Wang et al. (2016) injected dsRNA targeting a chitinase gene into S. litura larvae and observed degradation of the dsRNA in the hemolymph. Rajagopal et al. (2002) observed reduced expression of a gene encoding midgut aminopeptidase N after injection of dsRNA into S. litura larvae. Rana et al. (2020) reduced expression of a chitin synthase gene (Accession No. JN003621) in S. litura by feeding a hairpin RNA targeting the gene transcript. Sarkar and Kalia (2023) reduced expression of a gene encoding juvenile hormone epoxide hydrolase in S. litura. Xiao and Lu (2022) reduced the expression of two genes encoding cytochrome P450 monooxygenases (CYP6AE43 and CYP6AE480) in S. litura and increased the larval susceptibility to pyrethroids that were substrates for the P450. Zhao et al. (2013) targeted a gene encoding the enzyme catalase (siltCAT, Accession number: JQ663444) in S. litura.

Apone et al. (2014) expressed a dsRNA targeting a gene encoding corticotropin-releasing factor-like binding receptor, a type of diuretic hormone receptor (Accession No. ACJ06650), in transgenic tobacco. When fed to cotton leafworm (Spodoptera littoralis, Lepidoptera) larvae, they observed significantly increased larval mortality. Khalil et al. (2023) targeted aquaporin genes (Accession Nos. MN883561 and MN883562) in S. littoralis with dsRNA by injection and observed larval and pupal mortality, deformed pupae and adults and prolonged development. Kotwica-Rolinska et al. (2013) targeted a gene designated period in S. littoralis with dsRNA.

All of the reports described above for Spodoptera species used conventional dsRNA molecules, not the RNA molecules of the present invention. The RNA molecules of the invention can be used to reduce expression in Spodoptera frugiperda and other Spodoptera species, including of all of the target genes referred to in those publications, in particular the hairpin or ledRNA molecules of the present invention having G:U basepairs when expressed in planta, or without G:U basepairs when applied topically or expressed in yeast. The RNA molecules optionally have the Δ22, 423 or 424 modification, or combinations thereof, preferably the Δ22 modification to produce asymmetric RNA molecules, without, or preferably with, G:U basepairs, and preferably with a length of at least 100 basepairs in the dsRNA region(s). It is expected those molecules will provide enhanced knockdown of the target gene activity. The insect pests are thereby controlled for plants such as maize (Zea mays), sorghum (Sorghum species and hybrids), rice, wheat, soybean and cotton plants.

Plutella xylostella

The Diamondback moth (DBM, Plutella xylostella, Lepidoptera) is a pest of numerous plant crops worldwide, especially cruciferous vegetables such as Brassica species. DBM is responsible for severe yield losses and control costs have been estimated as $4-5 billion per year for the world economy (Furlong et al., 2013). Conventional RNAi has been used to reduce gene expression in DBM. For example, Bautista et al. (2009) used droplet feeding with a dsRNA targeting a gene encoding a cytochrome P450 protein, CYP6BG1, and observed knockdown of transcript. This was associated with reduced resistance to the chemical pesticide, permethrin. Chaitanya et al. (2017) tested dsRNAs targeting genes encoding juvenile hormone epoxide hydrolase (JHEH, Accession nos. KT595673, NM001305537) or ecdysone receptor (KT626046, EF417852) using a painted cabbage leaf disc feeding assay and observed gene down-regulation and larval mortality. Chandra et al. (2018b) used a similar bioassay to reduce expression of a gene encoding acetylcholinesterase (AChE) using a concatamerised dsRNA made up of a multiple of a shorter dsRNA, thereby interrupting the action of neurotransmitters and causing increased larval mortality. Chen et al. (2021) identified dsRNA-degrading enzymes (dsRNases) such as dsRNase1 (Accession no. MZ517187), dsRNase2 (MZ517188), dsRNase3 (MZ517189) and dsRNase4 (MZ517190) in the larval gut and hemolymph as a key factor affecting the stability of dsRNA in DBM. Inhibition of these dsRNases by RNAi increased the efficiency of knockdown of a chitinase target gene. Ellango et al. (2018) also used the painted cabbage leaf feeding assay to reduce expression of a tyrosine hydroxylase (Accession no. JN410829) as a target gene, involved in the biosynthesis of 3,4-dihydroxyphenylalanine (DOPA), a precursor for agents that function in neurotransmission, melanization, sclerotization of the cuticle and immune responses in insects. Fu et al. (2019) expressed hairpin RNAs targeting genes encoding arginine kinase (AK, Accession no. HQ327310) and integrin β1 subunit (Accession no. GQ178290) and observed delayed development, lower pupal weight and some mortality.

Gong et al. (2011) reduced expression of a gene encoding Rieske iron-sulfur protein (RISP, Accession no. EU815629). Guo et al. (2015c) targeted a gene encoding an ABC transporter (ABCH1, Accession no. KP260785) in DBM and observed larval and pupal lethal phenotypes in both susceptible and Cry1Ac-resistant P. xylostella strains. Hu et al. (2014) reduced expression of a gene encoding a cytochrome P450 gene (CYP321E1) by injecting a dsRNA. Jiang et al. (2022) expressed dsRNAs in a bacterial Bacillus thuringiensis (Bt) strain and observed reduced reproduction. Rana et al. (2020) targeted a gene encoding chitin synthase A (Accession no. AB271784) in DBM. Zhou et al. (2019) reduced expression of a gene encoding an ABC transporter (ABCB1, Accession no. MK613451), resulting in significantly reduced larval susceptibility to Cry1Ac toxin. WO2021/231791 describes compositions and methods for controlling DBM or FAW, including numerous candidate target genes therein (SEQ ID NOs 115-1682 in WO2021/231791).

All of the reports described above for Plutella xylostella used conventional dsRNA molecules, not the RNA molecules of the present invention. The RNA molecules of the invention can be used to reduce expression in Plutella xylostella, including of all of the target genes referred to in those publications, in particular the hairpin or ledRNA molecules of the present invention having G:U basepairs when expressed in planta, or without G:U basepairs when applied topically or expressed in yeast. The RNA molecules optionally have the Δ22, Δ23 or Δ24 modification, or combinations thereof, preferably the Δ22 modification to produce asymmetric RNA molecules, without, or preferably with, G:U basepairs, and preferably with a length of at least 100 basepairs in the dsRNA region(s). It is expected those molecules will provide enhanced knockdown of the target gene activity. The insect pests are thereby controlled for plants such as plants in the Brassicaceae family such as Brassica species.

Diabrotica virgifera virgifera and Other Diabrotica Species.

The RNA molecules of the present invention can also be used to reduce gene expression and thereby control insect pests in the order Coleoptera. For example, the western corn rootworm (WCR, Diabrotica virgifera virgifera) is an important pest of corn, particularly in North America and Europe. WCR larvae feed on the roots of the plants, reducing yield through reduced water and nutrient uptake. Currently in North America, transgenic corn varieties expressing Bt proteins are used to manage WCR, although additional protections such as use of RNAi are desired. The potential for species-specific control of the pest makes use of RNAi attractive. In June 2017, the EPA in the USA approved the first in planta RNAi product against insects for commercial use, namely transgenic corn expressing a hairpin RNA targeting the WCR snf7 gene in combination with Bt proteins. Snf7 is involved in protein trafficking, sorting and transport in the larval cells. Studies conducted in the field demonstrated that the RNAi construct in combination with the multiple Bt genes protected the transgenic corn not only against WCR but also northern corn rootworm (NCR; Diabrotica barberi) (Head et al., 2017).

Use of RNAi in WCR has been reviewed by Fishilevich et al. (2016) and Darlington et al. (2022). The latter review lists many target genes (Table 1) that have been shown to be effectively silenced in WCR, for example by addition of dsRNAs to an artificial diet. All of those genes can be targeted with the RNA molecules of the present invention. Baum et al. (2007) first reported that ingestion of a dsRNA targeting a gene encoding v-ATPase-A elicited a silencing response in WCR larvae, leading to mortality. Following that, Bolognesi et al. (2012) reported that dsRNA having a length of at least 60 basepairs were effective for silencing activity in artificial diet bioassays for WCR. In contrast, siRNA molecules of 21 nucleotides in length by themselves were not effective to reduce gene expression when ingested in artificial diet bioassays. Using a 240-nucleotide dsRNA, reduction of Snf7 gene expression in tissues beyond the midgut i.e. spread of the silencing, was shown to occur within 24 h after dsRNA ingestion. Li et al. (2015c) also reported that longer dsRNA molecules initiated RNAi in WCR larvae and adults, targeting a gene encoding v-ATPase subunit C. In contrast, 15-, 25- and 27-basepair siRNAs and pooled 21-basepair RNAs did not reduce transcript levels or cause larval mortality when added to the artificial diet or injected. Miyata et al. (2014) also tested dsRNAs of different lengths.

Fishilevich et al. (2019) targeted a gene encoding a Troponin I protein (WupA), involved in muscle contraction, and observed growth inhibition within two days. Knockdown of the protein resulted in significant food accumulation in the hindgut due to a loss of peristaltic motion of the alimentary canal. Hu et al. (2016), Hu et al. (2019) and Hu et al. (2020) targeted ssj1 (Accession no. KU562965) or ssj2 (Accession no. KU562966) genes encoding membrane proteins associated with smooth septate junctions (SSJ) which are required for intestinal barrier function and observed mRNA suppression, larval growth inhibition and mortality. Hu et al. (2016) also identified a cohort of 35 WCR genes that were effectively targeted with dsRNA, including protb (Accession no. KU756279), pat3 (KU756280) and rps10 (KU756281), all of which can be targeted with the RNA molecules of the present invention. Khajuria et al. (2015) targeted two genes, brahma (Brm) and hunchback (Hb), and observed reduced egg viability after female beetles were exposed to the dsRNA in artificial diet feeding assays. Brahma encodes an SWI/SNF ATP-dependent chromatin remodeler and hunchback encodes a zinc finger transcription factor. Velez et al. (2017) also targeted the brahma (Brm) and hunchback (Hb) genes in one generation of WCR and observed effects in a second, progeny generation, terming this parental RNAi.

Niu et al. (2017) targeted two genes, Vgr encoding a vitellogenin receptor (Accession no. KY373243) and Bol encoding a midgut factor boule (KY373244) which both have functions in WSC reproduction. Vélez et al. 2020 tested a dsRNA targeting the sec23 gene, encoding a coatomer protein which is a component of the (COPII) complex that mediates ER-Golgi transport and observed 85% reduction of mRNA with 40% reduction in Sec23 protein levels. The Sec23 gene was sensitive to RNAi in both larvae and adults. They commented that complete protein knockdown was not necessary to achieve insect mortality. Rangasamy and Siegfried (2011) also targeted the gene encoding v-ATPase in WCR and observed some larval mortality. Wu et al. (2017) reduced expression of several genes involved in the RNAi response, namely Ago1, Ago3, Aubergine and Dcr1.

Uptake of dsRNA in WCR larvae may be assisted by two SID1 homologs, SilA (LOC114327414) and SilC (LOC114340333) (Miyata et al., 2014), although the data are not clear. DsRNA may also be taken up in WCR through clathrin-mediated endocytosis (CME, Pinheiro et al., 2018). The siRNA silencing pathway is initiated in WCR when long dsRNA (>60 basepairs) is cleaved within the cytoplasm into siRNAs by the RNase III family ribonuclease Dicer-2 (LOC114339627) together with the dsRNA-binding protein R2D2 (LOC114342393). Even though conventional dsRNA molecules are effective in WCR and spread of the RNAi response in the larvae has been shown, no RdRp homolog is found in its genome and small RNA sequencing after exposure to dsRNA found no evidence of secondary siRNA production in WCR. Nevertheless, WCR exhibits a strong systemic response to dsRNA (Alves et al., 2010; Li et al., 2018).

All of the reports described above for Diabrotica species used conventional dsRNA molecules, not the RNA molecules of the present invention. The RNA molecules of the invention can be used to reduce expression in Diabrotica virgifera virgifera and other Diabrotica species, including of all of the target genes referred to in those publications, in particular the hairpin or ledRNA molecules of the present invention having G:U basepairs when expressed in planta, or without G:U basepairs when applied topically or expressed in yeast. The RNA molecules optionally have the Δ22, Δ23 or Δ24 modification, or combinations thereof, preferably the Δ22 modification to produce asymmetric RNA molecules, without, or preferably with, G:U basepairs, and preferably with a length of at least 100 basepairs in the dsRNA region(s). It is expected those molecules will provide enhanced knockdown of the target gene activity. The Diabrotica pests are thereby controlled for plants such as Zea mays.

### Colorado Potato Beetle

The Colorado potato beetle (CPB, Leptinotarsa decemlineata, Coleoptera) is a major insect pest of potato (Solanum tuberosum) globally, also damaging other solanaceous plants such as eggplant, pepper and tomato (Solanum lycopersicum). CPB is a voracious feeder on plant leaves during its larval stage and has a high fecundity with females laying 300-800 eggs each. CPB has a flexible life cycle that includes adult diapause and resistance to many insecticides has emerged. If left unmanaged, CPB can cause estimated yield losses of up to US$2.5 billion per year.

Ma et al. (2020) and Timani et al. (2023) reviewed the use of dsRNA in CPB, including the screening of RNAi target genes, RNAi delivery systems, and factors affecting RNAi efficiency in CPB. Table 1 of Ma et al. (2020) lists target genes for RNAi-based CPB control. Schoville et al. (2018) made available the genome sequence for CPB, providing for many candidate target genes. Palli (2014) reviewed steps toward development of dsRNA as a commercial insecticide for CPB. He commented in 2014 that more efficient methods for production and delivery of dsRNA needed to be developed for CPB. The present inventors expect that the RNA molecules of the invention described herein will be more efficacious than conventional dsRNA in reducing target gene expression and thereby for controlling CPB.

Cappelle et al. (2016) demonstrated uptake of conventional dsRNA in larvae through both a SID-mediated pathway and clathrin-mediated endocytosis, reducing expression of an α-amylose gene and a v-ATPase gene. Fu et al. (2016a) expressed two dsRNAs in E. coli, targeting a gene encoding juvenile hormone acid methyltransferase (JHAMT, Accession no. KP274881) and fed the bacteria to CPB. Fu et al. (2016b) reduced expression of five genes encoding insulin-like peptides (ILP, Accession nos. KP696394-KP696398) and observed effects on pupation. Fu et al. (2015) reduced expression of a gene (Accession no. AHH29249) encoding a nutrient amino-acid transporter. Guo et al. (2015a) assessed RNAi efficiency by double-stranded RNA (dsRNA) by targeting a gene transcript encoding S-adenosyl-L-homocysteine hydrolase (LdSAHase) and compared the effectiveness in first- to fourth-instar larvae of CPB. Ingesting dsRNA decreased the target gene expression, caused lethality, inhibited growth and impaired pupation in an instar- and concentration-dependent manner. Guo et al. (2015b) reduced expression of a HR3 gene encoding a transcription factor involved in ecdysone synthesis. Guo et al. (2018) expressed a hairpin RNA in transgenic potato plants, targeting a gene encoding juvenile hormone (JH) acid methyltransferase (JHAMT, Accession no. KP274881) and observed reduced gene expression and reproduction, even though the damage to foliage of the transgenic and control plants was similar after insect inoculation in initial experiments. He et al. (2020a) expressed a dsRNA from the chloroplast genome of potato plants, targeting a gene encoding a beta-actin and observed increased mortality, suppression of larval growth as well as reduction of target gene expression. He et al. (2020b) showed that dsRNA molecules that varied in sequence from the target gene sequence were still able to reduce expression of that target gene. He et al. (2022) tested different regions of a beta-actin gene from CPB by feeding 200-basepair dsRNAs and observed reduction of gene expression for all regions, with some more strongly reduced than others. Hussain et al. (2019) expressed a hairpin RNA in transgenic potato, targeting a gene encoding ecdysone receptor (Accession no. AB211192).

Ivashuta et al. (2015) analysed the uptake and processing of exogenous dsRNA in two coleopteran insects that are relatively sensitive to RNAi, WCR and CPB, and two lepidopteran insects, FAW and corn earworm (CEW, Helicoverpa zea), which are relatively resistant to RNAi. They demonstrated uptake of longer dsRNA molecules in WCR and CPB, but not in FAW and CEW. Kong et al. (2014) reduced expression of a gene encoding a cytochrome P450 monooxygenase (CYP314A1, accession no. KF044271) that catalyses the conversion of ecdysone (E) to 20-hydroxyecdysone (20E) in CPR. Liu et al. (2014) reduced expression of genes involved in ecdysone production, namely FTZ-F1-1, FTZ-F1-2 and Shd. Liu et al. (2021) silenced genes involved in N-glycosylation of proteins, including the genes STT3b, DAD1 and GCS1. Lu et al. (2015) expressed several dsRNAs in bacterial cells, targeting two genes encoding juvenile hormone epoxide hydrolase (JHEH). Petek et al. (2020) silenced a Mesh gene, alternatively named ssj2, which encodes a smooth septate junction protein important for structural integrity of the midgut epithelium in CPB. This was achieved by foliar spraying of bacterially-produced dsRNA onto potato plants in the field.

Rodrigues et al. (2021) developed a dsRNA molecule, designated Ledprona, which is a 490 bp dsRNA including 460 bp specific to the target transcript encoding a proteasome subunit beta type-5 (PSMB5, corresponding to nucleotides 450-927 of Accession No. XM_023158308.1) in CPB, designed for sprayable/topical application (WO2020/097414). San Miguel and Scott (2015) applied dsRNA to excised potato leaves in a feeding assay. The dsRNA targeted an actin gene transcript (Accession no. KJ577616). Larval mortality was observed for leaves treated with 297 bp or 208 bp dsRNA. Shukla et al. (2016) tested dsRNA targeting three housekeeping genes, encoding actin, Sec23-B or vATPase-B, in CPB as well as homologs in the lepidopteran Heliothis virescens. Spit et al. (2017) confirmed that knockdown of nuclease activity in the CPB gut enhanced RNAi efficiency in CPB. Swevers et al. (2013) identified numerous potential target genes in a CPB gut transcriptome. Yoon et al. (2016) screened 50 different target genes with potential functions in RNAi by exposing CPB cells to dsRNA, followed by incubation with dsRNA targeting a transcript encoding an inhibitor of apoptosis (IAP) (WO2020/069109). Yoon et al. (2018) identified a double-stranded RNA binding protein, Staufen, as required for the initiation of RNAi in coleopteran insects.

Zhang et al. (2015) silenced genes encoding a beta-actin, an essential cytoskeletal protein, and a protein designated as Shrub, also known as Vps32 or Snf7, an essential subunit of a protein complex involved in membrane re-modeling for vesicle transport. They pointed out that the plant's own Dicer endonuclease for producing small RNAs prevented the accumulation of high amounts of long dsRNA i.e. conventional dsRNA. They therefore expressed long dsRNA from the chloroplast genome in transformed (transplastomic) potato plants and observed high larval mortality after 5 days of feeding. Zhu et al. (2011) fed dsRNAs targeting five different target genes in CPB, namely genes encoding β-Actin (Accession no. EB761683), sec23 (FB778245), vacuolar ATP synthase subunit E (GM890243), vacuolar ATP synthase subunit B (FB778253) and coatomer subunit beta (CS675713). They observed reduced expression of all five target genes tested and significant mortality and reduced body weight gain in the treated insects.

All of the reports described above for Leptinotarsa decemlineata used conventional dsRNA molecules, not the RNA molecules of the present invention. The RNA molecules of the invention can be used to reduce expression in Leptinotarsa decemlineata, including of all of the target genes referred to in those publications, in particular the hairpin or ledRNA molecules of the present invention having G:U basepairs when expressed in planta, or without G:U basepairs when applied topically or expressed in yeast. The RNA molecules optionally have the Δ22, Δ23 or Δ24 modification, or combinations thereof, preferably the Δ22 modification to produce asymmetric RNA molecules, without, or preferably with, G:U basepairs, and preferably with a length of at least 100 basepairs in the dsRNA region(s). It is expected those molecules will provide enhanced knockdown of the target gene activity. The Leptinotarsa pests are thereby controlled for plants, including solanaceous plants such as potato.

Coleopteran Insect Pests Other than Diabrotica and Colorado Potato Beetle

RNAi has been used to reduce gene expression in Coleopteran insect pests other than the Diabrotica sp. and CPB described above. Dietary sensitivity of Coleopteran insects (beetles) to conventional dsRNA has been reviewed by Willow and Veromann (2021) and Kunte et al., (2020). There are now multiple reports of attempts to use conventional dsRNA in Coleopteran insects other than Diabrotica sp. and CPB. For example, Abd El Halim et al., (2016) observed that RNAi-mediated knockdown of a voltage gated sodium ion channel (TcNav) caused mortality in Tribolium castaneum. Bodemann et al., (2012) reduced expression of a gene encoding a salicyl alcohol oxidase in juveniles of the poplar leaf beetle, Chrysomela populi, and a juvenile hormone-binding protein in juveniles of the defensive glands of the related mustard leaf beetle, Phaedon cochleariae. Mehlhorn et al., (2021b) also targeted genes in Phaedon cochleariae by oral delivery, reporting that the most effective target genes with a strong, lethal phenotype at dsRNA doses as low as 300 ng/leaf disc, equal to 9.6 g/ha, were srp54k, rop, αSNAP, rpn7 and rpt3. Dhandapani et al., (2020) tested 48 candidate target genes for RNAi in the Asian long-horned beetle (ALB), Anoplophora glabripenni, and identified several effective target genes when larvae and adults were fed bacteria producing the dsRNA molecules. Guo et al., (2023) used dsRNA to test RNAi against nine housekeeping genes in the striped flea beetle Phyllotreta striolata (SFB, Fabricius), an insect pest that attacks Brassicaceae plants worldwide. These genes included PsVATPA, PsHSP90, PsEF1A, PsRPL6, PsRPS24, PsActin, PsTUBA and PsRPS18. A total of 24 other target genes were tested in SFB adults, of which reduction of Psα-COPI, Psβ-COPI, PsRPS18, Psy-COPI and PsArf1COPI expression caused significant mortality. Horn et al., (2022) observed persistent RNAi in progeny of the red flour beetle Tribolium castaneum that had been treated with long dsRNA molecules targeting the gene encoding homeodomain transcription factor Tc-Zen1. Knorr et al., (2018) used diet-based dsRNA assays to test 50 different genes for silencing in Tribolium castaneum and WCR. Transgenic maize plants expressing hairpin RNAs targeting genes encoding Rop, dre4 or RpII140 showed protection from WCR larval feeding damage. Kyre et al., (2019) provided an artificial diet containing dsRNA to southern pine beetles (Dendroctonus frontalis), as did Hollowell et al., (2023). Lu et al. (2020a, 2020b, 2021a, 2021b) and Guo et al. (2021) examined RNAi efficacy via oral delivery of dsRNAs targeting various genes in the 28-spotted potato ladybird, Henosepilachna vigintioctopunctata.

Pampolini and Rieske (2023) tested the use of dsRNA for the control of the emerald ash borer (EAB) Agrilus planipennis (Coleoptera: Buprestidae) by delivery through the plant. They observed dsRNA persistence in plant tissues 21 days after spraying. Powell et al., (2016) carried out a systemic RNAi study for gene down-regulation in the small hive beetle Aethina tumida (Coleoptera: Nitidulidae), a serious pest of the European honeybee Apis mellifera. Injection of dsRNAs targeting V-ATPase subunit A and Laccase 2 resulted in larval mortality, with suppression of transcript levels. Rodrigues et al., (2017a) tested RNAi in the Asian Longhorned Beetle (ALB) Anoplophora glabripennis, a serious invasive pest of forest trees, to identify target genes. Rodrigues et al., (2017b) developed a bioassay for oral delivery of dsRNA to larvae of the emerald ash borer (EAB, Agrilus planipennis), and reported that that dsRNA was transported and processed to siRNAs by EAB larvae within 72 h after ingestion. Feeding EAB neonate larvae with dsRNA targeting genes encoding an inhibitor of apoptosis (IAP) or COPI coatomer, β subunit) reduced the gene expression and caused mortality. Rodrigues et al., (2018) also targeted EAB, screening 13 candidate genes in neonate larvae and selecting the most effective target genes for further investigation. The two most efficient target genes selected, hsp encoding a heat shock 70-kDa protein 3 and shi, encoding a shibire protein, caused up to 90% mortality of larvae and adults.

Ulrich et al., (2015) carried out a screen of 5,000 candidate target genes in Tribolium castaneum and identified 100 genes that showed high levels of lethality both nine days after pupal and eleven days after larval dsRNA injection. Wang et al., (2019) investigated the effect of conventional dsRNA lengths on silencing efficacy in Tribolium castaneum and concluded that longer dsRNA molecules were more effective, concluding that molecules with lengths of 480 bp were about the same as 240 bp>120 bp>60 bp>>21 bp. 0.025 μg. Xu et al. (2021) applied dsRNA onto poplar leaves at a concentration of 8 ng/cm² and demonstrated 100% mortality when Plagiodera versicolora larvae were fed on the leaves. Zhang et al., (2019) painted a suspension of dsRNA-expressing bacteria on fresh willow leaves, resulted in approximately 40-80% mortality of Plagiodera versicolora larvae, an insect pest of Salicaceae plants worldwide, targeting genes including actin or a signal recognition particle protein 54k (SRP54).

The Family Curculionidae (true weevils) make up the largest coleopteran family and many species are plant pests. RNAi using conventional dsRNA has been shown to reduce gene expression in weevils. For example, Christiaens et al., (2016) demonstrated substantial mortality in the sweetpotato weevil Cylas brunneus after the insects were supplied an artificial diet containing dsRNA. The target genes prosα2, rps13 and snf7 were reduced in expression.

All of the reports described above for Coleopterans used conventional dsRNA molecules, not the RNA molecules of the present invention. The RNA molecules of the invention can be used to reduce expression in all of these insects, including of all of the target genes referred to in those publications, in particular the hairpin or ledRNA molecules of the present invention having G:U basepairs when expressed in planta, or without G:U basepairs when applied topically or expressed in yeast. The RNA molecules optionally have the Δ22, Δ23 or Δ24 modification, or combinations thereof, preferably the Δ22 modification to produce asymmetric RNA molecules, without, or preferably with, G:U basepairs, and preferably with a length of at least 100 basepairs in the dsRNA region(s). It is expected those molecules will provide enhanced knockdown of the target gene activity. The Coleopteran pests are thereby controlled after ingesting the RNA molecules.

### Cotton Bollweevil

The cotton bollweevil (CBW, Anthonomus grandis, Coleoptera: Curculionidae) is a major insect pest of cotton (Gossypium sp.) in Central and South America and potentially other cotton producing regions, particularly to the species G. hirsutum. The insect larvae directly feed on cotton flower buds and bolls and develop within these structures, thereby causing significant economic losses in infested areas. CBW has a high reproductive capacity and survival rate, leading to rapid infestations that can reduce the yield of cotton lint by 75-100% if left uncontrolled. Current control methods, primarily repeated spraying with insecticides to control adult weevils, are costly and inefficient due to the endophytic nature of CBW larvae as well as being environmentally hazardous. Since the early developmental stages are mostly protected inside the cotton bolls, contact insecticides have virtually no effect on those stages. Insect resistance to the insecticides is also a problem. Currently, there are no commercial transgenic cotton events capable of controlling CBW. There are a few published reports of attempts to generate such plants, for example with RNAi, or using RNAi for reverse genetics to down-regulate target gene expression in CBW.

Firmino et al., (2013) analysed the transcriptome of several developmental stages of CBW, producing a data set of more than 20,000 contigs, as a source for target genes for RNAi. No RdRP gene was found. Injection of dsRNA targeting a chitin synthase gene resulted in malformed larvae that did not develop properly. Firmino et al., (2020) targeted expression of a laccase 2 gene by microinjection of dsRNA. Garcia et al., (2017) reduced expression of gut RNAses and observed increased efficacy of orally-delivered dsRNA targeting a chitin synthase II transcript. They concluded that the gut RNAses contributed to the poor performance of orally-delivered dsRNA in CBW. Gillet et al., (2017) combined dsRNA with a chimeric peptide transduction domain-dsRNA binding domain protein (PTD-DRBD) and observed increased uptake of the ribonucleoprotein particles by oral delivery. Macedo et al., (2017) reduced expression of a gene encoding chitin synthase II and observed reduction of oviposition and mortality of adult insects. Moreira-Pinto et al., (2021) reduced expression of a Relish gene and other genes related to immune responses in the insect. Ribeiro et al (2022) produced transgenic cotton plants that expressed a stabilised RNAi molecule based on a viroid sequence, targeting genes coding for chitin synthase 2, vitellogenin (Trewitt et al., 1992) and ecdysis-triggering hormone receptor in CBW. When the T1 cotton plants were challenged with fertilised CBW females, mortality at about 70% was observed in oviposited yolks. In adult insects fed on transgenic plant tissues, expression of chitin synthase II and vitellogenin was reduced in larvae and adults, respectively. Developmental delays and abnormalities were also observed in these individuals. Salvador et al., (2021) fed dsRNAs separately targeting six different A. grandis genes for two weeks and observed reduction of gene expression, particularly for genes encoding α-amylase and cytochrome P450 proteins. US2017/0029843 reported the use of dsRNA targeting chitin synthase II and vitellogenin genes in cotton plants. Vasquez investigated the potential of chitosan-tripolyphosphate (CS-TPP) and polyethylenimine (PEI) nanoparticles as a dsRNA carrier system to improve RNAi efficiency in CBW, targeting genes encoding juvenile hormone diol kinase (JHDK), juvenile hormone epoxide hydrolase (JHEH), and methyl farnesoate hydrolase (MFE).

All of the reports described above for Anthonomus grandis used conventional dsRNA molecules, not the RNA molecules of the present invention. The RNA molecules of the invention can be used to reduce expression in Anthonomus grandis, including of all of the target genes referred to in those publications, in particular the hairpin or ledRNA molecules of the present invention having G:U basepairs when expressed in planta, or without G:U basepairs when applied topically or expressed in yeast. The RNA molecules optionally have the Δ22, Δ23 or Δ24 modification, or combinations thereof, preferably the Δ22 modification to produce asymmetric RNA molecules, without, or preferably with, G:U basepairs, and preferably with a length of at least 100 basepairs in the dsRNA region(s). It is expected those molecules will provide enhanced knockdown of the target gene activity. The CBW pests are thereby controlled for cotton plants.

### Hemipteran and Dipteran Insects

The Hemiptera are sucking insect pests, including aphids, whiteflies, planthoppers and psyllids that cause extensive damage to vegetable and other plants, and bedbugs and other blood-sucking bugs such as the kissing bug Rhodnius prolixus which are important in human health. The Hemiptera include economically important pests such as the whitefly Bemisia tabaci, the brown planthopper Nilaparvata lugens, the white-backed planthopper Sogatella furcifera, the small brown plant hopper Laodelphax striatellus, the potato/tomato psyllid Bactericera cockerelli, the Asian citrus psyllid Diaphorini citri, and the pea aphid Acyrthosiphon pisum, all of which may be controlled with the RNA molecules of the present invention. Christiaens and Smagghe (2014) and Jain et al, (2020) reviewed the use of RNAi in Hemipteran insects, including the reports by Hajeri et al., (2014), Thakur et al., (2014), Wan et al., (2014), Wuriyanghan et al., (2013), Zha et al., (2011).

The Diptera are flying insects that include pests such as mosquitoes and fruit flies. Belles (2010) reviewed use of RNAi in insects, including flies, for example in Drosophila. Maktura et al., (2021) also reviewed the use of dsRNA in fruit flies (Diptera: Tephritidae), referring to many candidate target genes that have been analysed for control of fruit fly. Shi et al., (2022) reviewed the potential for RNAi-based control of fruit fly. In the Tephritidae species Anastrepha fraterculus, about 55 core RNAi genes have been identified, such as those related to microRNA (Dcr-1, Ago-1, Drosha), siRNA (Dcr-2, R2D2 and Ago-2) and piwi-interacting RNA (Ago-3 and Piwi) (Dias et al., 2019). The fruit fly Ceratitis capitata is a polyphagous and cosmopolitan species which damages many plant species worldwide. Reduction in gene expression in fruit fly has been reported after oral delivery of dsRNA targeting genes encoding Rp119 in Bactrocera dorsalis (Chen et al., 2015), Transformer-2 in the striped fruit fly Zeugodacus scutellate (Al Baki et al., 2020) or Bactrocera dorsalis (Liu et al, 2015b), Tssk1 and Text in the Queensland fruit fly Bactrocera tryoni (Cruz et al., 2018), 143 different target genes in the South American fruit fly Anastrepha fraterculus (Dias et al., 2019), eight spermatogenesis related candidate genes in Bactrocera dorsalis (Dong et al., 2016), trehalose-6-phosphate synthase (TPS) by transgenic plant-mediated RNAi to control Bemisia tabaci (Gong et al., 2021), juvenile hormone esterase in Bemisia tabaci (Grover et al., 2019), ribosomal protein Rp119, V type ATPase subunit D, the fatty acid elongase Noa and a small GTPase Rab11 by bacterial expression in Bactrocera dorsalis (Li et al., 2011). Upadhyay et al., (2011) targeted genes encoding an actin ortholog, ADP/ATP translocase, α-tubulin, ribosomal protein L9 (RPL9) and V-ATPase subunit A in the whitefly Bemisia tabaci by oral delivery and observed mortality of the flies.

RNAi has also been shown to reduce gene expression in mosquitoes, including after oral delivery of the dsRNA. Abbasi et al., (2020) reduced gene expression using ‘paperclip” RNA molecules. Brizzee et al., (2023) generated yeast cells expressing a hairpin RNA targeting a GGT gene, encoding a gamma-glutamyl transpeptidase which is well conserved in multiple species of mosquitoes, and showed mortality of Culex quinquefasciatus female larvae which ingested the yeast cells. Coy et al., (2012) fed Aedes aegypti with dsRNA targeting the gene encoding vacuolar ATPase subunit A and observed gene knockdown. Cui et al., (2022) expressed mosquito miRNAs in an entomopathogenic fungus to reduce gene expression in Aedes aegypti and Galleria mellonella larvae. Dhandapani et al., (2019) developed nanoparticles to enhance RNAi efficiency in Aedes aegypti. Estep et al., (2016) targeted genes encoding ribosomal transcripts RPS6 and RPL26 and observed significant reductions in fecundity in Aedes aegypti. Khalil et al., (2023b) generated RNAi-mediated mortality of Culex quinquefasciatus by targeting genes encoding dynamin, ras opposite (ROP), juvenile hormone acid methyl transferase (JHAMT) or HMG-COA reductase (HMGR) using two delivery methods with potential for field application. Kumar et al., (2016) used chitosan/dsRNA nanoparticles for reducing expression of a gene encoding wing development vestigial (vg) in Aedes aegypti. Lopez et al., (2019) and Singh et al., (2013) targeted genes encoding chitin synthases A and B in Aedes mosquitoes.

Other sucking insect pests can be treated with the RNA molecules of the invention. Conventional dsRNA has been used in sucking insects, for example: the lygus bug Lygus lineolaris (Allen et al., 2012), the two-spotted spider mite Tetranychus urticae (Bensoussan et al., 2022), the brown planthopper Nilaparvata lugens by targeting genes encoding a sodium channel protein Nach-like, autophagy protein 5 or a V-type proton ATPase catalytic subunit (Dang et al., 2022) or chitin synthetase A (Lyu et al., 2023), the mirid bug Apolygus lucorum (Dong et al., 2023), the brown marmorated stink bug Halyomorpha halys (Finetti et al., 2023; Ghosh et al., 2018), the Asian citrus psyllid Diaphorina citri (Galdeano et al., 2017; Saberi et al., 2024; Thakre et al., 2024), five cytochrome P450 family 4 proteins in Diaphorina citri (Killiny et al., 2014), the white-backed planthopper Sogatella furcifera (Guo et al., 2023b; Liu et al. 2022; Wan et al., 2014; Wang et al., 2019b), the small brown plant hopper Laodelphax striatellus (He et al., 2018), three different species (Kaplanoglu et al 2022), the green plant bug Apolygus lucorum (Qiao et al., 2023b), the mirid Adelphocoris suturalis (Qin et al., 2022), the tick Ixodes scapularis (Soares et al., 2005), the western flower thrip Frankliniella occidentalis (Venkatesh et al., 2023; Wu et al., 2022), the corn planthopper Peregrinus maidis (Wang et al., 2023c), the herbivorous mites Tetranychus urticae, Tetranychus evansi and Tetranychus ludeni by reducing expression of genes encoding beta-actin (Wu et al., 2023), and the potato/tomato psyllid, Bactericerca cockerelli (Wuriyanghan et al., 2011). Several of these studies used dsRNA produced in transgenic plants to deliver the dsRNA molecules. Yu et al., (2023) used nanoparticles encasing dsRNA improve the susceptibility of Nilaparvata lugens to imidacloprid.

All of the reports described above for Hemipteran and Dipteran insects used conventional dsRNA molecules, not the RNA molecules of the present invention. The RNA molecules of the invention can be used to reduce expression in all of these insects, including the target genes referred to in those publications. The hairpin and ledRNA molecules of the present invention can be generated with or without G:U basepairs and applied in a bait, for example when expressed in bacteria or yeast cells. The RNA molecules optionally have the Δ22, Δ23 or Δ24 modification, or combinations thereof, preferably the Δ22 modification to produce asymmetric RNA molecules. These asymmetric RNA molecules either do not have, or have, G:U basepairs and a length of at least 100 basepairs in the dsRNA region(s). It is expected those molecules will provide enhanced knockdown of the target gene activity. The insect pests are thereby controlled after ingesting the RNA molecules, either as adult insects or in the larval stage.

### Use of RNA Molecules to Provide Resistance to Plants Against Aphids

Aphids are insects of the Order Hemiptera, superfamily Aphidoidea, Family Aphididae. They are pests of plants that feed by sucking sap from the phloem and to some extent from the xylem, especially from young growing tissues in a plant. About 5,000 species of aphid have been described with about 450 species damaging food and fibre crops. Altogether, aphids are serious, worldwide pests for a wide range of plant species. Many aphid species are monophagous i.e. feeding on only one plant species or a few related species. Others, like the green peach aphid, feed on hundreds of plant species across many families. Their life cycle typically involves flightless females giving live birth to female nymphs, reproducing parthenogenically without involvement of males, or through a sexual cycle in autumn which results in producing eggs. Aphid species that are serious plant pests include Aphis fabae (black bean aphid), Metopolophium dirhodum (rose-grain aphid), Myzus persicae (peach-potato aphid), Rhopalosiphum padi (bird cherry-oat aphid), Aphis glycines (soybean aphid), Aphis gossypii (cotton or melon aphid), Macrosiphum euphorbiae (potato aphid)), Aulacorthum solani (foxglove aphid), Macrosiphoniella sanborni (chrysanthemum aphid), Lipaphis erysimi (mustard aphid), Brevicoryne brassicae, Acyrthosiphon pisum (pea aphid), Acyrthosiphon kondoi (bluegreen aphid), Pentalonia nigronervosa (banana aphid) and Sitobion avenae (grain aphid). Each of these aphid species can be controlled with the RNA molecules of the present invention, particularly the hairpin RNA or ledRNA molecules having G:U basepairs and/or the Δ22 modification.

Reduction of target gene activity by conventional RNAi through oral feeding of RNA molecules has been demonstrated in aphids, reviewed by Yu et al. (2016) and Zhang et al. (2022b), although effects appear to be mainly reduced fecundity. Bhatia and Bhattacharya (2017) targeted a transcript encoding cuticular protein (CP) in Myzus persicae through expression of dsRNA molecules in transgenic Arabidopsis thaliana plants, observing reduced fecundity. Bhatia et al. (2012) targeted a serine protease gene in M. persicae. Coleman et al. (2015) expressed dsRNAs in transgenic A. thaliana plants against genes encoding Rack1, MpC002 or MpPIntO2 (Mp2) and observed reduction of gene expression and reduced reproduction in M. persicae. Dhatwalia et al. (2021) targeted a transcript encoding sucrase 1 in the mustard aphid, Lipaphis erysimi, by expressing a hairpin RNA in transgenic Brassica juncea plants, also observing reduced fecundity. Gao et al. (2020) observed increased larval and adult mortality and reduced total fecundity in both cotton- and cucumber-specialised aphids Aphis gossypii by reducing expression of a cytochrome P450 gene, AgoCYP6CY19. Gong et al. (2014) targeted a gene encoding a carboxylesterase protein in Aphis gossypii that resulted in reduced resistance to an organophosphorus insecticide. Guo et al. (2014) targeted nine different genes in M. persicae by expressing hairpin RNAs in transgenic tobacco plants and observed some aphicidal activity. Jekayinoluwa et al. (2021) expressed a hairpin RNA targeting an AChE gene of Pentalonia nigronervosa in banana and plantain plants and observed reduction in aphid populations growing on the transgenic plants i.e. reduced fecundity. Ma et al. (2023) applied sprays of hairpin RNAs produced in E. coli, targeting vacuolar-type ATPase subunits D (ATPase-D) and G (ATPase-G) of green peach aphid and, as for others, observed reduced aphid reproduction. Mao and Zeng (2014) expressed a hairpin RNA in transgenic tobacco against a hunchback gene of M. persicae and observed reduced insect reproduction, indicating some knockdown of the target gene. Mulot et al. (2018) expressed a hairpin RNA in transgenic A. thaliana plants against a gene encoding a membrane-bound Ephrin receptor (Eph), an aphid protein involved in the transmission of a virus by Myzus persicae, and observed reduced virus levels in the aphids and transmission to plants. Murtaza et al. (2022) expressed a hairpin RNA in transgenic potato plants against a macrophage inhibitory factor (MIF1) gene of M. persicae and observed increased insect mortality, indicating some knockdown of the target gene. Niu et al. (2019) targeted various genes in the pea aphid Acyrthosiphon pisum by topical delivery of dsRNA molecules. Pitino et al. (2011) targeted two genes, salivary protein C002 and Rack1, by feeding and reported 30-40% reduction in transcript levels and a moderate reduction in the number of nymphs produced by treated aphids. Shakesby et al. (2009) reported that diet-delivered dsRNA directed to transcripts encoding aquaporin reduced expression by more than 50%. Tzin et al. (2015) expressed hairpin RNAs in N. benthamiana leaves targeting three M. persicae genes encoding aquaporin, sucrase and a sugar transporter individually and in combination and found that the combined treatment yielded a greater effect on the aphid hemolymph osmotic pressure and body weight than the individual dsRNA treatments. Ullah et al. (2020) reported that reduction of expression of a gene encoding chitin synthase 1 (CHS1) caused increased mortality and decreased fecundity in Aphis gossypii. Xie et al. (2022) targeted a gene encoding a zinc finger protein (MPZC3H10) in M. persicae. Ye et al. (2019) targeted a gene encoding chitin synthase in the pea aphid, Acyrthosiphon pisum, and observed reduction of gene expression. Zhang et al. (2013b) identified potential RNAi target genes in the grain aphid, Sitobion avenae. Zhao et al. (2018) targeted a chitin synthase gene (CHS1, Accession No JQ246352 for Aphis glycines) of the grain aphid Sitobion avenae by expressing a hairpin RNA against the CHS1 transcript in transgenic wheat and observed reduced aphid reproduction and increased grain yield.

While these studies suggest that conventional RNAi might be used to suppress aphid populations, they have not shown effects sufficient for field control (Ghodke et al., 2019). All of the reports described above for aphids used conventional dsRNA molecules, not the RNA molecules of the present invention. The RNA molecules of the invention can be used to reduce target gene expression in aphids, including of all of the target genes referred to in those publications, in particular the hairpin or ledRNA molecules of the present invention having G:U basepairs when expressed in planta, or without G:U basepairs when applied topically. In an embodiment of the present invention, the target RNA molecule is not a transcript from a Rack1 gene or a C002 gene in Myzus persicae. The C002 protein is an aphid salivary gland protein which is essential for aphid feeding on its host plant (Mutti et al., 2006; Mutti et al., 2008). Rack1 is an intracellular receptor that binds activated protein kinase C, an enzyme primarily involved in signal transduction cascades (McCahill et al., 2002; Seddas et al., 2004). The RNA molecules optionally have the Δ22, Δ23 or Δ24 modification, or combinations thereof, preferably the Δ22 modification to produce asymmetric RNA molecules, without, or preferably with, G:U basepairs, and preferably with a length of at least 100 basepairs in the dsRNA region(s). It is expected those molecules will provide enhanced knockdown of the target gene activity. The aphids are thereby controlled.

### Ants

Ants (Hymenoptera: Formicidae) are important pests both for agriculture and in urban areas. They generally do not feed directly on plants, yet can have significant impact on pastures, orchards and nurseries. The Argentine ant, Linepithema humile, the tawny crazy ant Nylanderia fulva, and the red imported fireant, Solenopsis invicta, are significant invasive pests that can be controlled with the RNA molecules of the invention, for example by incorporating the molecules into a bait that is collected by the ants and taken to their nest. Only liquids can pass into the crop or midgut of adult ants but ant larvae, on the other hand, have no such constriction and are able to ingest solid baits. Allen et al., (2021) has reviewed the prospects for using dsRNA molecules to control ants, including for colony-level control, referring to target gene sequences. Ant genomes have genes similar to SID1, involved in the uptake and systemic spread of RNA molecules. Cheng et al., (2015) targeted a gene encoding a chemosensory protein 9 (Si-CSP9) in the fire ant Solenopsis invicta (Hymenoptera: Formicidae). Lu et al., (2009) targeted a gene encoding vitellogenin receptor (VgR) in Solenopsis invicta, abolishing egg formation. Wang et al., (2024) targeted six different genes in S. invicta by oral delivery and observed increased silencing when the RNA molecules were formulated into liposomes or expressed in bacteria, particularly for a gene encoding v-ATPase subunit E. Choi et al., (2012) and patent U.S. Pat. No. 8,575,328 report targeting of a gene encoding PBAN. Meng et al., (2020) silenced genes in the tawny crazy ant (Nylanderia fulva) by oral delivery of bacteria expressing RNA molecules, observing silencing of genes encoding COP beta, involved in protein trafficking, and ArgK, a cellular energy reserve regulatory gene in invertebrates.

All of the reports described above for ants used conventional dsRNA molecules, not the RNA molecules of the present invention. The RNA molecules of the invention can be used to reduce expression in all of these ants, including the target genes referred to in those publications. The hairpin and ledRNA molecules of the present invention can be generated with or without G:U basepairs and applied in a bait, for example when expressed in bacteria or yeast cells. The RNA molecules optionally have the Δ22, Δ23 or Δ24 modification, or combinations thereof, preferably the Δ22 modification to produce asymmetric RNA molecules. These either do not have, or have, G:U basepairs and a length of at least 100 basepairs in the dsRNA region(s). It is expected those molecules will provide enhanced knockdown of the target gene activity. The ant pests are thereby controlled after ingesting the RNA molecules.

### Target RNA Molecules in Insect Pests

The RNA molecules of the present disclosure can be used to decrease expression and/or amount of any target RNA molecule(s) in an insect cell, tissue, organ or insect, including the target transcripts from genes in the insect species as described herein. This applies not just to Helicoverpa armigera and Spodoptera species and other species described above but also to homologs of the target RNA molecules in other insect species. The target RNA molecules may be from genes that control insect activity, behaviour, reproduction, growth and/or development. For example, numerous target genes are listed in the review by Kola et al. (2015), including genes involved in the digestive system, defence, metabolism and key proteins in the insect life cycle. All of these can be targeted for reducing activity with the RNA molecules of the invention. Specific genes are described as follows.

### Pheromone Biosynthesis Activating Neuropeptide (PBAN) and Receptors as Target Genes

Neuropeptides play an important role in the regulation of many physiological processes in insects including growth, development, reproduction, feeding, courtship, olfaction and circadian rhythm. Neuropeptides are therefore considered to be a type of peptide hormones. A wide range of neuropeptides have been described in insects (Xu et al., 2016b) and homologs can be readily identified. Pheromone Biosynthesis Activating Neuropeptide (PBAN) is one of these neuropeptides in insects that regulates sex pheromone biosynthesis from the pheromone gland, being produced in the suboesophageal ganglion following adult emergence in a species dependent manner, and therefore required for reproduction. PBAN also has effects earlier in development in insect larvae. PBAN is recognised by its G-protein coupled transmembrane receptor, designated as PBAN-R. Both classes of genes can be targeted by the RNA molecules of the invention.

As used herein, a PBAN target gene encodes a pheromone biosynthesis activating neuropeptide. A great many PBAN genes and neuropeptides in insects are well known, for example from H. armigera (Accession No. XM_021343523, XP_021199198, AAQ82626), Helicoverpa zea (XP_047039942), Heliothis virescens (AAO20095), Heliothis peltigera (COHL17), Spodoptera exigua (AXY04289, AAT64424, CAH0698246), Spodoptera frugiperda (XP_035457345), Spodoptera litura (XP_022832611), Spodoptera littoralis (Q95P48), Mythimna loreyi (KAJ8704896), Mythimna separata (KAJ8706590), Bombyx mori (BAA03755, BAA05954), Manduca sexta (XP_030038326, KAG6465309), Chrysodeixis includens (CAH0602349) and Ostrinia furnacalis (XP_028168133). In an embodiment, the target RNA transcript has at least 95% identity to a cDNA encoding at least one of these naturally-occurring PBAN proteins. In an embodiment, the target RNA transcript has at least 50% identity to SEQ ID NO: 8, and/or encodes an PBAN protein having at least 50% identity to SEQ ID NO: 7.

PBAN receptor genes are also well known, for example Accession No. GSE175545 for S. frugiperda (Park and Vatanparast, 2022).

### Acetylcholinesterase (AChE) Target Genes

Acetylcholinesterase is an enzyme expressed in the central nervous system of insects. The enzyme functions by hydrolysing acetylcholine into choline and acetic acid (Oakeshott et al., 2005) and thereby has an important role in termination of the signal transmission process in the nervous system. Besides neurotransmission, AChE is thought to have other roles in cellular processes such as modulation of cellular interactions, apoptosis, cell adhesion and the genesis of synaptic connections (Soreq and Seidman, 2001; Zhang and Shi, 2002). The expression of AChE has been reported in the insect body wall, anterior body structures and appendages during embryonic development, and strong expression occurs in longitudinal glia and glial cells during larval development as well as nerve tissues (Bicker et al., 2004). In many insects including H. armigera, two homologous AChE genes are present, namely AChE1 and AChE2 (Lee et al., 2006). AChE1 transcripts are much more abundant than AChE2, and therefore the AChE1 gene was selected by the present inventors for targeting by the modified hairpin RNA molecules. Kumar et al. (2009) silenced the AChE1 gene by feeding H. armigera larvae an artificial diet supplemented with chemically synthesized, sense and antisense hybrid RNA-DNA molecules of 19 basepairs in length that had been annealed, corresponding to nucleotides 1657-1675 of SEQ ID NO: 16 herein. High concentrations of the molecules at 25-50 nM resulted in slowed development and a reduction in survival of the larvae from about 85% to 60%.

As used herein, an AChE target gene encodes an acetylcholinesterase protein. A great many AChE genes and their proteins in insects are well known, for example from H. armigera (Accession Nos. AY142325, AF3629793, AEK27380, SEQ ID NO: 16 herein), Spodoptera frugiperda (KAF9815860), Spodoptera exigua (AZB49079), Spodoptera litura (XP_022825018.1), Sesamia inferens (QFG71777), Mythimna separata (KAJ8727616), Chrysodeixis includens (CAH0605819), Chilo suppressalis (RVE44827, CAH0693177), Manduca sexta (KAG6450933), Chilo auricilius (AIK27531), Pectinophora gossypiella (pink bollworm, XP_049870090), Scirpophaga incertulas (AOW44164), Bombyx mori (ABY50089), Plutella xylostella (Diamond backed moth, XP_048478879), Tuta absoluta (Tomato pinworm, KAJ2952127), and Leptinotarsa decemlineata (CPB, Q27677). In an embodiment, the target RNA transcript has at least 95% identity to a cDNA encoding at least one of these naturally-occurring AChE proteins. In an embodiment, the target RNA transcript has at least 60% identity to SEQ ID NO: 16, and/or encodes an AChE protein having at least 60% identity to SEQ ID NO: 15.

### Ecdysone Receptor

Another gene transcript that can be targeted by RNA molecules of the invention is one encoding Ecdysone Receptor (EcR). The H. armigera gene encoding EcR is designated herein as Ha1. The EcR protein is a master regulator of insect development. It is involved in the regulation of all developmental stages of the insect life cycle, in particular embryogenesis, hatching, insect growth, molting and metamorphosis. The EcR protein forms a heterodimer with another protein, ultraspiracle (Usp), to form a nuclear receptor for the steroid hormone α-ecdysone and its hydroxylated derivative, 20-hydroxyecdysone, referred to herein collectively as ecdysone. The receptor is required for the steroid hormone signalling pathway that controls insect development (Thummel, 1995; Riddiford et al., 2000). When bound to ecdysone, the EcR-Usp complex functions as an active transcription factor to regulate several genes at the top of a hierarchy of genes as well as multiple, more downstream genes. The receptor complex does this by binding to DNA elements upstream of the target genes at different developmental stages (Uyehara and Mckay, 2019). The early genes that are regulated by EcR include those encoding the Broad-Complex, E74A and E75B proteins, each of which in turn also regulate downstream genes. The early genes activate a group of late genes which regulate metamorphosis during insect development including the processes of cell proliferation and differentiation, cell death and cuticle formation. The EcR gene is also induced directly by ecdysone, providing an autoregulatory loop that increases the level of receptor protein in response to its ligand.

In Drosophila, a single gene encodes three isoforms of EcR, namely EcR-A, -B1 and -B2, through the use of two promoters and alternative RNA splicing. These differ in their N-terminal domains and have different temporal and spatial expression patterns, indicating distinct roles in development (Talbot et al., 1993; Li and Bender, 2000; Hu et al., 2003). Mutations in the EcR locus that inactivate all three isoforms are embryonically lethal (Bender et al., 1997). Both dsRNA- and artificial miRNA-mediated silencing of the gene encoding EcR in H. armigera (GenBank Accession No. EU526831) have resulted in increased larval mortality and moulting defects (Yogindran and Rajam 2016, Yogindran and Rajam 2021, Zhu et al., 2012).

As used herein, an EcR target gene encodes an EcR protein. A great many EcR genes and their proteins in insects are well known, for example from H. armigera (SEQ ID NO: 22 herein, encoding an EcR polypeptide with the amino acid sequence SEQ ID NO: 21, Accession No. KY328717.1, XP_021181320, XP_021181318), Helicoverpa zea (XP_047021243, XP_047021241, XP_047021244, XP_047021242), Heliothis virescens (O18473), Spodoptera frugiperda (XP_035436715, XP_035436713, XP_035436716), Spodoptera exigua (ACA30302), Spodoptera littoralis (CAB3516113, ADO64595), Spodoptera litura (XP_022819528, ABX79143, AFK27930, XP_022819527), Chrysodeixis includens (CAD0196875), Agrotis ipsilon (AGA17965), Manduca sexta (XP_030038451, XP_030038409, P49883), Chilo suppressalis (RVE49011, BAC11714), Bombyx mori (NP_001166846, NP_001037331), and Plutella xylostella (KAG7307565, XP_037973366, ABQ81864). In an embodiment, the target RNA transcript has at least 95% identity to a cDNA encoding at least one of these naturally-occurring EcR proteins. In an embodiment, the target RNA transcript has at least 80% identity to SEQ ID NO: 22, and/or encodes an AChE protein having at least 80% identity to SEQ ID NO: 21.

### Coatomer Beta Subunit

Another gene transcript in insects that can be targeted by RNA molecules of the invention is one encoding a coatomer subunit such as the coatomer beta (β-COPI) subunit, which is a protein that together with the other coatomer subunits functions in the Golgi apparatus of insect cells. The Golgi complex functions to modify, transport, sort and package proteins and lipids into vesicles which are delivered to various cellular compartments such as the plasma membrane, the extracellular medium and the endosomal/lysosomal compartments, particularly between the Golgi complex and the endoplasmic reticulum (Kondylis and Rabouille, 2009). Normal functioning of the Golgi complex involves the coating of vesicles with the Coatomer protein complex (COPI). Other than its role in protein transport, COPI is also involved in cell division and lipid homeostasis (Beck et al., 2009; Beller et al., 2008). COPI includes seven subunits (α, β, β′, γ, δ, ε, ξ), each of which, when absent in insects, reduce fitness and increase mortality rates. In Drosophila, loss-of-function mutants for COPI subunits have several secretory defects which are lethal in embryos (Jayaram et al., 2008). β-COPI is a subunit of COPI and is essential for the transport of proteins from the endoplasmic reticulum to the Golgi complex (Pepperkok et al., 1993). Down-regulation of the gene encoding β-COPI in the spider mite Tetranychus urticae (Trombidiformes) through artificial feeding of in vitro synthesised dsRNA of 513 bp in length resulted in 65% mortality after 5 days (Kwon et al., 2013). Colorado potato beetles (Leptinotarsa decemlineata, Coleoptera) fed with dsRNA targeting the gene encoding β-COPI showed reduction in its mRNA and an increase in mortality (Zhu et al., 2011). That dsRNA was 228 bp in length and had been synthesized in E. coli or by in vitro synthesis. Mao et al. (2015) reduced expression of the coatomer β and v-ATPase A genes in H. armigera using siRNA feeding-based assays, using a mixture of siRNAs having 21 canonical basepairs supplied at 10 μg/cm². However, 5 μg/cm² of siRNA failed to cause significant lethality in treated larvae. These results demonstrate that targeting of β-COP1 through RNAi can disrupt the integrity of the Golgi complex and disturb protein secretion impacting insect fitness.

The H. armigera gene encoding coatomer beta subunit is designated herein as Ha2. A nucleotide sequence for a cDNA corresponding to the mRNA encoding H. armigera coatomer beta subunit was obtained from Genbank Accession No. XM_021339008.1, provided herein as SEQ ID NO: 28, encoding a polypeptide with the amino acid sequence SEQ ID NO: 27.

As used herein, a coatomer target gene encodes a coatomer protein, for example a coatomer beta target gene encodes a coatomer-beta protein. A great many coatomer beta genes and their proteins in insects are well known in the art, for example H. armigera, Spodoptera exigua (Accession No. CAH0703668, KAH9643528, KAF9416125), Spodoptera frugiperda (XP_035450692, XP_035449935, KAF9807635), Spodoptera littoralis (CAB3517958), Spodoptera litura (XP_022832393, XP_022832392), Mythimna separata (KAJ8737274), Mythimna loreyi (KAJ8737424), Chrysodeixis includens (CAH0578336), Tuta absoluta (KAJ2954654), Chilo suppressalis (CAH0402194, RVE51053), Bombyx mori (NP_001166610), Manduca sexta (XP_037299737), Plutella xylostella (XP_048488619), Ostrinia furnacalis (XP_028176981) and Anopheles sinensis (KFB46946). In an embodiment, the target RNA transcript has at least 95% identity to a cDNA encoding at least one of these naturally-occurring COPI proteins. In an embodiment, the target RNA transcript has at least 60% identity to SEQ ID NO: 28, and/or encodes an COPI protein having at least 60% identity to SEQ ID NO: 27.

### Molt-Regulating Transcription Factor (HR3)

As described above, the steroid hormone ecdysone controls multiple developmental transitions in insects including the larval to adult metamorphosis. The ecdysone induced moult-regulating transcription factor HR3 is a member of the nuclear receptor superfamily which, when expressed, triggers a cascade of gene expression changes required for moulting (Palli et al., 1992). In Drosophila and Manduca sexta, HR3 expression is induced by ecdysone at various stages in development including the transition from larvae to prepupae (Koelle et al., 1992; Parvy et al., 2014). HR3 inhibits early response factors while at the same time inducing the expression of βFtz-F1, a key factor in the pre-pupal to pupal transition (Parvy et al., 2014) and other factors. In Drosophila, HR3 is also required for normal embryonic development of the central nervous system and for hatching (Carney et al., 1997). HR3 is particularly important for the formation of tissues such as wings and the epidermis. Null HR3 mutants are embryonic lethal, associated with defects in the development of the nervous system and muscle (Carney et al., 1997). In H. armigera, the HR3 polypeptide of 556 amino acids is widely expressed in a variety of tissues including the midgut, epidermis and fat bodies (Zhao et al., 2004). In Locusta migratoria nymphs, injecting a dsRNA targeting HR3 inhibited molting and led to death of the insect (Zhao et al., 2018). Expression of the HaHR3 gene was reduced in H. armigera by feeding any one of four dsRNAs produced in E. coli, or feeding transgenic tobacco expressing a hairpin RNA, resulting in developmental deformity and larval lethality (Xiong et al., 2013). The hairpin RNA targeted the region of nucleotides 1-450 of SEQ ID NO: 34 herein. In a different study with six different target genes, Jaiwal et al. (2020) fed second instar H. armigera larvae on an artificial diet containing dsRNA produced by in vitro transcription against the HaHR3 gene, resulting in various deformities and larval mortality. The dsRNA targeted the region of nucleotides 934-1440 of SEQ ID NO: 34 herein.

The H. armigera gene encoding HR3 is designated herein as Ha3. A nucleotide sequence for a cDNA corresponding to the mRNA encoding H. armigera HR3 was obtained from Genbank Accession No. FJ009448, provided herein as SEQ ID NO: 34, encoding a polypeptide with the amino acid sequence SEQ ID NO: 33.

As used herein, a HR3 target gene encodes a HR3 protein. A great many HR3 genes and their proteins in insects are well known in the art, for example H. armigera (FJ009448, AAK14384, XP_049700350, XP_021186799), Helicoverpa zea (XP_047038126), Mythimna separata (KAJ8704523), Mythimna loreyi (KAJ8707127), Chrysodeixis includens (CAH0599797, CAH0599794), Spodoptera exigua (CAH0698000), Ostrinia furnacalis (XP_028177799, XP_028177800), Spodoptera litura (XP_022816977, XP_022816980), Spodoptera frugiperda (XP_050558298, XP_050558300, XP_050558296), Chilo suppressalis (RVE53591), Bombyx mori (XP_012551819, NP_001037012), Manduca sexta (XP_030022276, Q08882, XP_030022274), Cydia pomonella (Codling moth, XP_061726681), Leguminivora glycinivorella (XP_048002294), Plutella xylostella (XP_011564618) and Choristoneura fumiferana (AAC47163). In an embodiment, the target RNA transcript has at least 95% identity to a cDNA encoding at least one of these naturally-occurring HR3 proteins. In an embodiment, the target RNA transcript has at least 80% identity to SEQ ID NO: 34, and/or encodes a HR3 protein having at least 80% identity to SEQ ID NO: 33.

v-ATPase

The vacuolar-type H-ATPase, commonly referred to as the v-ATPase, catalyses the hydrolysis of ATP to ADP and phosphate and uses the energy associated with this reaction to drive ions across cell membranes, thereby regulating pH in multiple organelles (Wieczorek et al., 1989, Wieczorek et al., 2009). The complex drives H⁺ from inside the cell to the lumen and thereby enables cells to import ions such as K⁺ and other molecules, including osmotically obliged water. V-ATPase is a complex of multiple subunits including the VO and V1 sub-complexes that is highly conserved in insects and is found in nearly all epithelial tissues including the gut, salivary glands, testes, ovarioles, testes and Malpighian tubules (Wieczorek et al., 2003; Zeng et al., 2021). In Lepidopteran insects, the V1 complex of the plasma membrane v-ATPase contains eight different subunits designated A to H, whereas the VO complex consists of the four different subunits a, c, d and e (Merzendorfer et al., 2000). V-ATPase is also associated with alkalisation of the midgut in Lepidoptera to pH values as high as 11 or 12 (Azuma et al., 1995). Early work in Drosophila demonstrated lethality to larvae when v-ATPase was mutated or disrupted (Davies et al., 1996; Allan et al., 2005). South American tomato pinworm (Tuta absoluta, Lepidoptera) larvae fed on leaves exogenously applied with 50 μg/cm² of dsRNA, produced by in vitro transcription, targeting the v-ATPase B gene had significantly increased mortality rate after 3 days feeding (Ramkumar et al., 2021). Mao et al. (2015) targeted the mRNA encoding v-ATPase A in H. armigera larvae with four different siRNAs each having 19 basepairs, exogenously applied on the surface of tobacco leaves and fed to the larvae. This resulted in a reduction in size and an increase in mortality after 10 days treatment. The siRNAs were synthesised as sense and antisense RNAs and targeted the regions corresponding to nucleotides 553-570, 115-1171, 1288-1306 and 1579-1597 of SEQ ID NO: 40 herein. In another study, Jin et al. (2015) transformed chloroplasts of tobacco plants with a genetic construct to express a short hairpin RNA targeting the v-ATPase A transcript of H. armigera and observed reduced larval growth and pupation rates. The short hairpin had 19 canonical basepairs and targeted the region of the mRNA corresponding to nucleotides 711-729 of SEQ ID NO: 40 herein.

The H. armigera gene encoding v-ATPase A is designated herein as Ha4. A nucleotide sequence for a cDNA corresponding to the mRNA encoding H. armigera v-ATPase A was obtained from Genbank Accession No. XM_021325374.1 (LOC110369820), provided herein as SEQ ID NO: 40, encoding a polypeptide with the amino acid sequence SEQ ID NO: 39.

As used herein, a v-ATPase target gene encodes a v-ATPase protein, for example a v-ATPase A target gene encodes a v-ATPase A protein. A great many v-ATPase A genes and their proteins in insects are well known in the art, for example H. armigera (Accession Nos. XM_021325374, XP_021181049), Spodoptera exigua (AQQ72785, CAH0686039), Chrysodeixis includens (CAH0625698), Manduca sexta (XP_030028570), Spodoptera frugiperda (KAF9808893, UAJ21578, XP_035437427), Mythimna loreyi (KAJ8719870), Bombyx mori (NP_001091829), Spodoptera littoralis (CAB3507174), Spodoptera litura (XP_022826560), Chilo suppressalis (AXF48683), Ostrinia furnacalis (XP_028155919), Plutella xylostella (XP_048483376), Tuta absoluta (KAJ2937492), Cydia pomonella (XP_061717726), Monomorium pharaonic (Pharoah ant, XP_012533527), Solenopsis invicta (XP_025991763) and Linepithema humile (XP_012229966). In an embodiment, the target RNA transcript has at least 95% identity to a cDNA encoding at least one of these naturally-occurring v-ATPase A proteins. In an embodiment, the target RNA transcript has at least 60% identity to SEQ ID NO: 40, and/or encodes a HR3 protein having at least 60% identity to SEQ ID NO: 39.

### Trypsin-Like Serine Protease

Trypsin-like serine proteases are major digestive enzymes found in the gut of insect larvae (Sui et al., 2009; Zhu et al., 2003). Reduction in tryspin-like proteases results in poor protein digestion and a lack of amino acids for growth. The expression of the gene encoding the protease in H. armigera is tissue-specific: the mRNA is expressed in the midgut and not in the head-thorax, integument, fat body and haemocytes from 5th instar larvae (Sui et al., 2009). In H. armigera, the protease is negatively regulated through a miRNA, namely har-miR-2002b. The miRNA is expressed at high levels up until the 4th instar after which the miRNA expression level decreases and the tryspin-like protease expression increases (Jayachandran et al., 2013). Over-expressing har-miR-2002b by supplying a mimic miRNA to the food of H. armigera larvae resulted in a number of developmental changes within the insect. Insects fed the mimic miRNA displayed a reduction in weight, a delay in entering the pupal stage and reduced fecundity compared to control insects (Jayachandran et al., 2013). The present inventors realised from the observation that H. armigera development is sensitive to fluctuations in expression level of the trypsin-like protease that this gene is a preferred target for testing the hpRNA[G:U] molecule.

The H. armigera gene encoding trypsin-like serine protease is designated herein as Ha5. A nucleotide sequence for a cDNA corresponding to the mRNA encoding H. armigera trypsin-like serine protease was obtained from Genbank Accession No. EU874846.1, transcript variant X1, provided herein as SEQ ID NO: 46, encoding a polypeptide with the amino acid sequence SEQ ID NO: 45.

As used herein, a trypsin-like serine protease target gene encodes a trypsin-like serine protease protein. A great many trypsin-like serine protease genes and their proteins in insects are well known in the art, for example H. armigera (EU874846, XP_049694924, XP_049694926, XP_049694925, XP_049694927, PZC85445, PZC85440), Helicoverpa zea (XP_047022503, XP_047022502, XP_047041336), Mamestra configurata (ADM35107), Mythimna separata (KAJ8727488, KAJ8727491) and Mythimna loreyi (KAJ8733380). Related proteases include chymotrypsinogen B-like from Spodoptera frugiperda (isoform X3, XP_050562211), Helicoverpa armigera (XP_049694922), Manduca sexta (XP_030041352) and chymotrypsinogen A-like from Spodoptera frugiperda (XP_050562209). In an embodiment, the target RNA transcript has at least 95% identity to a cDNA encoding at least one of these naturally-occurring proteases. In an embodiment, the target RNA transcript has at least 35% identity to SEQ ID NO: 46, and/or encodes a protease having at least 35% identity to SEQ ID NO: 45.

### Synaptic Vesicle Glycoprotein

One of the protein families involved in neurotransmission in animals is the synaptic vesicle glycoprotein 2 (SV2) family. The SV2 proteins are transmembrane proteins found on secretory vesicles, including synaptic vesicles, which facilitate exocytosis of synaptic vesicles by rendering them responsive to calcium. SV2 proteins are members of the major facilitator superfamily, a large family of membrane transporters expressed widely throughout bacteria, archaea, and eukarya. SV2s have 12 transmembrane domains and cytosolic N- and C-termini. Drosophila species have multiple SV2 family orthologs including the so-called SV2-like proteins. The present inventors are not aware of any reports of down-regulation of the genes encoding SV2 with RNAi.

The H. armigera gene encoding SV2 is designated herein as Ha6. A nucleotide sequence for a cDNA corresponding to the mRNA encoding H. armigera SV2 was obtained from Genbank Accession No. XP_021181756.1, transcript variant X2, provided herein as SEQ ID NO: 52, encoding a polypeptide with the amino acid sequence SEQ ID NO: 51.

As used herein, a SV2 target gene encodes a SV2 protein. A great many SV2 genes and their proteins in insects are well known in the art, for example H. armigera (SEQ ID NO: 52), Helicoverpa zea (XP_047029381), Spodoptera frugiperda (XP_050561208), Spodoptera litura (XP_022824561), Spodoptera exigua (KAF9417036), Mythimna loreyi (KAJ8718566), Mythimna separata (KAJ8714631), Trichoplusia ni (XP_026741123), Chrysodeixis includens (CAH0588102), Manduca sexta (XP_030034902), Bombyx mori (XP_004929625), Chilo suppressalis (RVE52607), Tuta absoluta (KAJ2949542), Ostrinia furnacalis (XP_028175157), Cydia pomonella (XP_061719505), Leguminivora glycinivorella (XP_04799593), Plutella xylostella (XP_037971922, KAG7300183), Tribolium castaneum (XP_015836306), Culex quinquefasciatus (XP_038110245) and Anopheles coustani (XP_058130024). In an embodiment, the target RNA transcript has at least 95% identity to a cDNA encoding at least one of these naturally-occurring SV2 proteins. In an embodiment, the target RNA transcript has at least 60% identity to SEQ ID NO: 52, and/or encodes a SV2 protein having at least 60% identity to SEQ ID NO: 51.

### Troponin C

Troponin is a complex comprised of three subunits designated troponins C, I and T which are important for controlling muscle contraction and relaxation in invertebrates (Eldred et al., 2014). The Troponin C subunit senses changes in the level of calcium, thereby inducing conformational changes that trigger contraction events (Herranz et al., 2004). Troponin C is expressed throughout the insect with the highest levels displayed in the intestinal tract, head and feet (Lan et al., 2018). In the silkworm Bombyx mori, the multiple genes encoding troponin C are expressed in the muscular tissues of the silkworm, including portions of the head, the Malpighian tubule, the body wall and the gut (Chen et al., 2008). In Drosophila, reduced expression of the genes encoding troponin C impacted muscle contraction and ability to fly (Eldred et al., 2014). Larvae of the green rice leafhopper, Nephotettix cincticeps (Homoptera), injected with dsRNA of about 477 basepairs targeting the mRNA encoding troponin C showed a variety of defects impacting fitness, including survival, feeding capacity and weight gain (Lan et al., 2018).

The H. armigera gene encoding troponin C is designated herein as Ha7. A nucleotide sequence for a cDNA corresponding to the mRNA encoding H. armigera troponin C was obtained from Genbank Accession No. XM_021340134.1, provided herein as SEQ ID NO: 58, encoding a polypeptide with the amino acid sequence SEQ ID NO: 57.

As used herein, a troponin C target gene encodes a troponin C protein. A great many troponin C genes and their proteins in insects are well known in the art, for example H. armigera, Ostrinia furnacalis (XP_028169949), Spodoptera exigua (KAH9639352), Cydia pomonella (XP_061719366), Leguminivora glycinivorella (XP_047992585), Bombyx mori (NP_001040443), Mythimna separata (KAJ8712288), Plutella xylostella (XP_011557268), Spodoptera littoralis (CAB3509521), Tuta absoluta (KAJ2945582) and Musca domestica (XP_005186180). In an embodiment, the target RNA transcript has at least 95% identity to a cDNA encoding at least one of these naturally-occurring troponin C proteins. In an embodiment, the target RNA transcript has at least 80% identity to SEQ ID NO: 58, and/or encodes a troponin C protein having at least 80% identity to SEQ ID NO: 57.

### Titin

The protein titin, the largest known endogenous protein in vertebrates, is involved in the elasticity and structural integrity of myofibrils and thereby has key structural and mechanical roles in cardiac and skeletal muscle in vertebrates. The human titin gene contains 364 exons over a length of more than 38 kb, coding for a protein of about 4200 kDa (Chauveau et al., 2014). The D-titin gene in Drosophila spans about 110 kb in the genome and encodes a protein of almost 2000 kDa (Machado and Andrew, 2000). The gene is expressed in all striated muscle cells in the insect and the protein localises to chromosomes and to sarcomeres. Titin transcripts are differentially spliced, particularly in regions encoding the tandem-Ig and PEVK domains, giving rise to many isoforms with different extensible properties. Loss of titin protein impacts chromosomal integrity during mitosis whereby mutant embryos display polyploidy, chromosome fragmentation and irregular condensation (Machado and Andrew, 2000). Defects in titin also result in myoblast fusion and a failure for midgut constrictions to form, resulting in insect death.

The H. armigera gene encoding titin is designated herein as Ha8. A nucleotide sequence for a cDNA corresponding to the mRNA encoding H. armigera titin was obtained from Genbank Accession No. XM_021340134.1, provided herein as SEQ ID NO: 64, encoding a polypeptide with the amino acid sequence SEQ ID NO: 63.

As used herein, a titin target gene encodes a titin protein. A great many titin genes and their proteins in insects are well known in the art, for example different isoforms in H. armigera (XP_021196691, XP_021196693, PZC73107, SEQ ID NO: 63), Helicoverpa zea (XP_047030239, XP_047030234, XP_047030238), Spodoptera exigua (CAH0692116, KAH9635504), Chrysodeixis includens (CAD0201093), Spodoptera frugiperda (XP_050555046, KAF9796832), Spodoptera litura (XP_022814079, XP_022814078, XP_022814074, XP_022814077), Spodoptera littoralis (CAB3508883), Manduca sexta (XP_030030519, XP_030030523, XP_030030521, XP_030030525), Ostrinia furnacalis (XP_028162558, XP_028162560, XP_028162559, XP_028162561), Chilo suppressalis (RVE52870, CAH2980593), Cydia pomonella (XP_061716648, XP_061716650, XP_061716649, XP_061716651), Bombyx mori (XP_037868403) and Plutella xylostella (XP_048478596, XP_048478595). In an embodiment, the target RNA transcript has at least 95% identity to a cDNA encoding at least one of these naturally-occurring titin proteins. In an embodiment, the target RNA transcript has at least 40% identity to SEQ ID NO: 64, and/or encodes a titin protein having at least 40% identity to SEQ ID NO: 63.

All of the above-mentioned genes may be targeted by the RNA molecules of the invention, particularly the ledRNA molecules having G:U basepairs and/or the Δ22 modification when expressed in planta or for oral delivery. It is expected that these molecules will have increased efficacy in reducing target gene expression compared to the conventional dsRNA molecules. Homologs of the target genes in other insect species may also be targeted. In view of the availability of genome sequences, for example for S. frugiperda (Gouin et al, 2017), such homologs are readily identified. In an embodiment for each of the target genes, the target transcript and/or the target protein has at least 50% identity to the sequence of the listed accession number, preferably at least 60% identity, more preferably at least 70% or 75% identity, even more preferably at least 80% or 85% identity, most preferably at least 90%, 95% or even 99% identity to the sequence of the listed accession number. All of these genes may be targeted with the RNA molecules of the present invention, particularly the hairpin RNA and ledRNA molecules having G:U basepairs and/or the Δ22 modification, preferably with a length of at least 100 basepairs in the dsRNA region(s).

Arthropods Other than Insects

The RNA molecules of the invention can be used to reduce genes expression in arthropods other than insects, for example in spider mites (Bensoussan et al., 2020), Varroa mites and ticks. Mites belong to the subphylum Chelicerata of the phylum Arthropoda. Conventional dsRNA has been used successfully in these arthropods, as reviewed by Niu et al., (2018), see for example Table 1 of that reference. Bensoussan et al., (2022) tested orally-delivered dsRNAs against target genes in the two-spotted spider mite, Tetranychus urticae, a herbivorous acari with an extremely wide host range that includes all major crops. They observed reduction of expression of genes encoding Rpn7, Snap α, Rop, Srp54, v-ATPase, Hsc70-3 and Rpt3. Suzuki et al., (2017) tested five different delivery methods in the spider mite, including several oral delivery methods, and Kwon et al., (2013, 2016) tested 42 different target genes including genes encoding coatomer subunits B and E. Khila and Grbic (2007) silenced the distal-less gene (Dll). Mondal et al., (2021) demonstrated transitive RNAi in mites. Wu et al., (2023) expressed dsRNA in the plastids of transplastomic tomato plants, targeting a conserved region of the gene transcript encoding beta-actin, and observed control of the spider mites. Dubey et al., (2017) used agroinfiltration-based expression of hairpin RNA in soybean leaves to reduce gene expression in spider mites.

Varroa mite (Varroa destructor) is a serious parasite of the honeybee (Apis mellifera). Conventional RNAi has been shown to be effective in the Varroa mite, for example Becchimanzi et al., (2024), Campbell et al., (2016), reviewed by Niu et al., (2018). Garbian et al., (2012) showed that dsRNA ingested by bees was transferred to the Varroa mite and from mites to a parasitized bee. Huang et al., (2015) targeted six candidate genes with dsRNA molecules, namely genes encoding the polypeptides Da, Pros26S, L8, L11, P0 and S13. Hunter et al., (2010) targeted a virus, Israeli Acute Paralysis Virus (LAPV) that infects honeybees in a trial simulating real field conditions. The dsRNA was shown to reduce bee mortality and improve the overall health of bees infected with IAPV. Leonard et al., (2020) used a symbiotic bacterium found in bee guts to express and deliver dsRNA to the insects—these could deliver dsRNA molecules targeting the Varroa mite. McGruddy et al., (2024) tested a dsRNA agent referred to as Vadescana (GreenLight Biosciences, Inc.) to assess the impact on Varroa destructor fitness following exposure to the dsRNA in the brood cells of the hive. Vadescana was designed to specifically target a gene encoding a calmodulin protein, which plays an important role in calcium regulation in the mite. The agent was reported to substantially reduce mite fertility.

All of the reports described above for arthropods other than insects used conventional dsRNA molecules, not the RNA molecules of the present invention. The RNA molecules of the invention can be used to reduce expression in all of these arthropods, including the target genes referred to in those publications. The hairpin and ledRNA molecules of the present invention can be generated with or without G:U basepairs and applied in a bait, for example when expressed in bacteria or yeast cells. The RNA molecules optionally have the Δ22, Δ23 or Δ24 modification, or combinations thereof, preferably the Δ22 modification to produce asymmetric RNA molecules. These asymmetric RNA molecules either do not have, or have, G:U basepairs and a length of at least 100 basepairs in the dsRNA region(s). It is expected those molecules will provide enhanced knockdown of the target gene activity. The arthropod pests are thereby controlled after ingesting the RNA molecules, either as adult arthropods or in the larval stage.

### Aquaculture

The RNA molecules of the invention can be used to reduce gene expression in crustacea and fish, for example to protect these organisms from pathogens such as viruses, or for sex control (Shpak et al., 2017). The use of conventional dsRNA molecules in aquaculture has been reviewed by Abo Al Ela et al., (2021), see for example Table 1 of that reference, and by Alam et al., (2023) who reviewed the use of conventional RNAi-based for therapy of diseases in shrimp, since bacterial, fungal and especially viral diseases can devastate shrimp populations, see Table 2 of that reference. Gong and Zhang (2021), Itsathitphaisarn et al., (2017) and Nguyen et al., (2018) also reviewed the use of RNAi to protect shrimp from viral disease. Viral infections are particularly threatening to crustacean aquaculture, especially shrimp such as Macrobrachium rosenbergii. Leigh et al., (2015) expressed dsRNA molecules in E. coli that targeted a hepandensovirus in Penaeus merguiensis and observed some reduction in the disease when the bacteria were delivered in the food. Similarly, Chimwai et al., (2016) reported reduction in virus in the shrimp Penaeus monodon when fed E. coli expressing a dsRNA targeting a densovirus. Further, Sarathi et al., (2008) expressed a dsRNA in E. coli targeting the VP28 gene of white spot virus and observed reduced disease in Penaeus monodon. Attasart et al., (2009; 2013) fed P. monodon or Litopenaeus vannamei with inactivated bacteria expressing dsRNA targeting genes encoding shrimp Rab7, a small GTPase protein, or STAT or the WSSV viral rr2 gene and observed a significant reduction of gene expression. Silencing of the shrimp Rab7 gene confers protection against WSSV, yellow head virus (YHV), Taura syndrome virus (TSV) and Laem-Singh virus (LSNV) (Ongvarrasopone et al., 2008, 2010, 2011). Somchai et al., (2016) expressed a hairpin RNA targeting the RNA-dependent RNA polymerase gene of yellow head virus in the microalgae Chlamydomonas reinhardtii to reduce disease in the whiteleg shrimp Penaeus vannamei. These results showed that dsRNA targeting proteases, polymerases or helicases of the virus genome can effectively inhibit the viral replication. Jonjaroen et al., (2024) reviewed the use of nanoparticles to deliver dsRNA molecules to shrimp, for example the use of composite polymer-clay nanoparticles (Suksai et al., 2025), chitosan particles (Ufaz et al., 2018), or virus-like particles (Ramos-Carreno et al., 2021).

### Target RNA Molecules in Fungal Pathogens

Fisher et al. (2012) estimated that fungal diseases of crop plants cause worldwide pre-harvest losses estimated at 10-23% of total crop and horticultural production, despite disease interventions, and a further 10-20% post-harvest loss. Current disease control strategies in the field include the planting of crops carrying in-bred disease resistance genes and the wide-spread spraying of antifungal chemicals. However, fungal pathogens often evolve rapidly to overcome the plant's disease resistance, or pathogen strains are selected that are resistant to the chemicals. There is therefore considerable interest in developing alternative control methods such as RNAi-mediated control, with the RNA molecules delivered either through spray on the plants or expressed in transgenic plants i.e. host-induced gene silencing (HIGS) through reducing expression of fungal genes.

As used herein, the term “fungus” or “fungi” means any organism in the eukaryotic kingdom Fungi, including the Class Oomycota (oomycetes). Fungi include the Eumycota, also known as true fungi or Eumycetes, the oomycetes such as in the order Peronosporales, and includes microorganisms such as yeasts and molds. A characteristic that places fungi in a different kingdom from plants, bacteria and some protists is the presence of chitin in their cell walls. Over 8,000 fungal species are known to be detrimental to plants and at least 300 that can be pathogenic to humans and other animals. In an embodiment of the present invention, the fungus is wild-type for Dicer i.e. it has not been modified in a gene encoding a Dicer.

Numerous studies have demonstrated that conventional dsRNAs and sRNAs, including miRNAs and siRNAs, can be taken up by fungal pathogens and induce gene silencing in the fungal cells. The dsRNAs can be produced in planta by introducing a gene encoding the dsRNA, termed host-induced gene silencing (HIGS), or the dsRNA can be applied topically, termed spray-induced gene silencing (SIGS). The use of dsRNAs to control fungal pathogens has been reviewed extensively, for example by Attia et al. (2022), Chen et al. (2023), Ciofini et al (2022), Fisher et al. (2012), Gebremichael et al. (2021), Ghag (2017), Maksimov et al. (2022), Mann et al. (2023), McLaughlin et al. (2023), McLoughlin et al. (2018), Nien et al. (2024), Niu et al. (2021), Padilla-Roji et al. (2023), Qiao et al., (2023), Secic and Kogel (2021), Singewar and Fladung (2023), Stukenbrock and Gurr (2023) and Wang et al. (2017). Ray et al. (2022) lists a wide range of genes that have been targeted with conventional dsRNA, often hairpin RNA, in HIGS and SIGS methods. Several reports describe that sRNAs can be taken up by fungal pathogens from plant cells through exosomes or extracellular vesicles that are absorbed by fungal cells through endocytic fusion with the cell membrane (Cai et al., 2018; Cai et al., 2019; Rutter and Innes, 2018). DsRNAs can also be applied topically through nanoparticles (Ray et al., 2022; Qiao et al., 2021; Wang et al., 2023b; WO2023/205424). Reported target genes in fungi include ones encoding acetyl-transferases (Gill et al., 2018; Hu et al., 2020b), chitin synthases (Saito et al., 2022), a range of genes (Yin et al., 2011), haustorial proteins (Yin et al., 2015), and protein kinases (Panwar et al., 2018; Qi et al., 2018; Zhu et al., 2017). For example, Regmi et al. (2023) identified candidate target genes in the necrotrophic pathogen Sclerotinia sclerotiorum. The RNA molecules of the present invention can be used to reduce gene expression for all of the genes described in those reviews and reports, based on the reports of gene silencing with conventional RNA molecules.

For fungal diseases, HIGS was first reported in the biotrophic powdery mildew fungus, Blumeria graminis (Nowara et al., 2010) and in Fusarium verticillioides (Tinoco et al., 2010). They tested 76 fungal candidate genes that were found to be expressed in planta during B. graminis infection (Zierold et al., 2005), and showed that transgenic barley and wheat that expressed conventional dsRNA targeting at least the effector gene Avra10 or 1,3-β-glucanosyltransferase (GTF1 and GTF2, Accession Nos EU646133 or J422119; HQ234876) could inhibit the development of B. graminis (Nowara et al., 2010). Koch et al. (2013) reported that HIGS was effective in controlling the necrotic fungal pathogen Fusarium graminearum in transgenic Arabidopsis and barley by targeting three genes encoding fungal cytochrome P450 lanosterol C-14a-demethylases (CYP51A, B and C).

Numerous reports followed for HIGS against fungi. Arias et al. (2015) expressed a hairpin RNA in transgenic peanut plants targeting several aflatoxin synthesis genes in the fungi Aspergillus flavus and Aspergillus parasiticus, namely the genes AFL2G_07223 (aflS or aflJ), AFL2G_07224 (aflR), AFL2G_07228 (aflC/pksA/pksL1), AFL2G_07731 (pes1) and AFL2G_05027 (aflatoxin efflux pump, aflep), and observed 60%-100% reduction in aflatoxin production in some transgenic plants. Bharti et al. (2017) expressed a hairpin RNA in transgenic tomato plants targeting several pathogenicity genes in the fungus Fusarium oxysporum f. sp. lycopersici (Fol) that causes vascular wilt of tomato, namely the genes encoding FOW2, a Zn(II)2Cys6 family putative transcription regulator, and chsV, a putative myosin motor, and a chitin synthase domain. They observed enhanced resistance to the disease in some transgenic plants. Similar results were observed by Singh et al. (2020) targeting a gene encoding ornithine decarboxylase (ODC) in Fol. Chauhan and Rajam (2024) also showed reduced pathogenicity of Fol in transgenic tomato, targeting genes encoding for the fasciclin-like proteins FoFLP1, FoFLP4 and FoFLP5. Tetorya and Rajam (2021) similarly targeted genes in Fol encoding peroxisomal biogenesis factor and β-1,3-glucanosyltransferase by expression of hairpin RNA in transgenic tomato. Chen et al. (2016) expressed a hairpin RNA in transgenic wheat plants targeting genes encoding β-1,3-glucan synthase (Accession No. KP195140), secreted lipase (Fgl1, KP195142), mitogen-activated protein (MAP) kinase (Fmk1, KP195139) and Chitin synthase V (ChsV, KP195141) in the head blight fungus Fusarium culmorum and observed reduced disease symptoms. Chen et al. (2022) expressed hairpin RNAs in transgenic rice targeting the transcription factor genes Com1 and Pro1, and the gene AspE that encodes a fungal-specific septin, in the fungus Ustilaginoidea virens that causes a smut disease in rice. Chen et al. (2023b) targeted genes encoding sugar transporters VdST3 or VdST12 of Verticillium dahliae in cotton plants and observed reduced fungal biomass and enhanced resistance against V. dahliae.

Dou et al. (2020) expressed dsRNA targeting genes encoding C-24 sterol methyltransferase (ERG6, Accession no. FOIG_12628T0) involved in ergosterol synthesis, cytochrome P450 lanosterol C-14α-demethylase (ERG11, Accession No. FOIG_00890T0), hydroxymethylglutaryl-CoA synthase (ERG13, FOIG_06149T0) and C-4 sterol methyl oxidase (ERG25, FOIG_11829T0) in transgenic banana and observed resistance to Fusarium oxysporum f.sp. cubense (Foc) Race 4. Fernandes et al. (2016) inhibited a SIX Gene Expression 1 (SGE1) gene transcript in Foc with hairpin RNA. Ghag et al. (2014) silenced two Foc genes, namely velvet and ftf1 encoding Fusarium transcription factor 1, by expression of dsRNA in transgenic banana and observed gene silencing and resistance to Fusarium wilt caused by Foc Race 1 in banana cv. Rasthali. Govindarajulu et al. (2015) expressed hairpin RNA targeting either the HAM34 or CES1 genes of the oomycete Bremia lactucae in transgenic lettuce and observed reduced growth and inhibition of sporulation of the pathogen and resistance to downey mildew disease. Gu et al. (2019) targeted a β2-tubulin gene transcript in Fusarium asiaticum as well as Botrytis cinerea, Magnaporthe oryzae and Colletotrichum truncatum, providing broad-spectrum antifungal activity. He et al. (2019) targeted two protein kinase genes Fg00677 and Fg08731, and cytochrome P450 lanosterol C14-α-demethylase (CYP51) encoding genes (CYP51A, CYP51B, and CYP51C) in Fusarium graminearum by expression in transgenic Brachypodium distachyon plants and observed strong resistance. Hofle et al. (2020) also targeted three CYP51 genes (CYP51A, CYP51B, CYP51C) in Fusarium graminearum (Fg) to inhibit fungal infection. Hu et al. (2015) inhibited three different genes (FOW2, FRP1, and OPR) in the hemi-biotrophic fungus F. oxysporum f. sp. conglutinans by expression of hairpin RNA in transgenic Arabidopsis plants.

Jahan et al. (2015) targeted genes in the potato late blight pathogen Phytophthora infestans, an oomycete, in particular the gene encoding a G protein β-subunit (GPB1) by expression of hairpin RNA in transgenic potato plants. Jiao et al. (2017) targeted a gene CaMKL1 (Accession no. number KF989486.1) encoding a CaMK-like protein kinase, required for full virulence of Puccinia striiformis f. sp. tritici. Johnson et al. (2018) observed reduced fumonisin mycotoxins produced in maize from Fusarium verticillioides by targeting a FUMI gene required for fumonisin production in the fungus. Mahto et al. (2020) targeted a transcript for a conidial morphology 1 (COM1) gene involved in the fungal conidial and appressorium formation in Colletotrichum gloeosporioides by expression of hairpin RNA in transgenic chili and tomato. Down-regulation of a gene encoding MAP-kinase (MAPK1, Accession no. DQ026061.1) or a cyclophilin (CYC1, Accession no. BU672663) by expression of hairpin RNAi constructs in wheat plants significantly suppressed the pathogenic fungus Puccinia triticina (Pt, Panwar et al., 2013a; Panwar et al., 2013b; Panwar et al., 2018). Perez et al. (2020) generated transgenic soybean plants expressing a hairpin RNA targeting a transcript from the CYP51B gene of Fusarium oxysporum and observed resistance to the pathogen. Qi et al. (2018) silenced a gene encoding protein kinase A (PKA) in Puccinia striiformis f. sp. tritici (Pst), the causative fungus for stripe rust, by expression of a hairpin RNA in transgenic wheat plants. Raruang et al. (2023) silenced a polygalacturonase (p2c) gene in Aspergillus flavus aflatoxin production by expression of a hairpin RNA in transgenic maize plants.

Song and Thomma (2018) targeted three genes encoding virulence factors Avel, Sge1 and NLP1 in Verticillium dahliae with hairpin RNA expressed in transgenic Arabidopsis. Su et al. (2017) targeted an AAC gene (Accession no. XP_009654735.1) of Verticillium dahlia by expression of hairpin RNA in transgenic Nicotiana benthamiana. Tiwari et al. (2017) targeted two genes, encoding pathogenicity factor MAP kinase (PMK1) homologues, RPMK1-1 and RPMK1-2, in Rhizoctonia solani, the causal agent of rice sheath blight disease, by expression of hairpin RNA in transgenic rice plants. Walker et al. (2023) reduced expression of a gene encoding Abhydrolase-3 in the causative agent of white mold, Sclerotinia sclerotiorum, by expression of hairpin RNA in transgenic Arabidopsis plants. Wang and Dean (2022) targeted six genes (CRZ1, PMC1, MAGB, LHS1, CYP51A, CYP51B) in Magnaporthe oryzae that causes rice blast disease by expression of hairpin RNAs in transgenic rice plants. Wang et al. (2022) reduced disease caused by Phytophthora capsica by targeting genes encoding cellulose synthase 3 (CesA3) or oxysterol binding protein 1 (OSBP1). Xu et al. (2020) targeted a gene encoding a polysaccharide deacetylase, a pathogenicity factor, in Puccinia striiformis f. sp. tritici (Pst), Pst_13661, in transgenic wheat plants. Xu et al. (2024) targeted RAS genes in Sclerotinia sclerotiorum. Zhang et al. (2016) targeted a gene encoding hygrophobin-1 (Accession no. VDAG_02273.1) in Verticillium dahliae by expressing a hairpin RNA in transgenic cotton. Zhu et al. (2017) targeted a gene PsFUZ7 in Pst by expression of hairpin RNA in transgenic wheat. WO2023/010022 describes candidate target genes in Botrytis such as Botrytis cinerea.

In addition, SIGS methods using the topical application of exogenous, conventional dsRNA have also been used to down-regulate genes in pathogenic fungi. This has been reviewed by Vetukuri et al. (2021) and Wang and Jin (2017). Cao et al. (2024) targeted three genes encoding β-tubulin (Tub), sterol 14α-demethylase (CYP51) and chitin synthase (Chs) in the powdery mildew pathogen Erysiphe quercicola by spraying dsRNA onto leaves of rubber trees. Degnan et al. (2022) targeted ten different genes, namely encoding β-tubulin, translation elongation factor 1α(EF1-α), acetyl-CoA transferase, cytochrome P450 (CYP450), mitogen activated protein kinase (MAPK), glycine cleavage system H (GCS-H), 28S ribosomal RNA, and three haustorial targets (HAUS01136, HAUS01215, and HAUS12890). Use of SIGS was also reported for fungal and oomycete pathogens including Fusarium graminearum (Kim et al., 2023; Koch et al., 2016; Koch et al., 2019), Botrytis cinerea (Wang et al., 2016c) and Phytophthora infestans (Kalyandurg et al., 2021; Wang et al., 2023). SIGS was also successfully reported for obligate biotrophs such as Phakopsora pachyrhizi (Hu et al., 2020), Podosphaera xanthii (Ruiz-Jimenez et al., 2021), Golovinomyces orontii and Erysiphe necator (McRae et al., 2023). Ouyang et al. (2023) used SIGS to successfully target a gene encoding RNA dependent RNA polymerase 1 (RDR1) in Fusarium oxysporum f.sp. lycopersici (Fol) that causes tomato wilt. Saito et al. (2022) reduced disease in soybean plants caused by Phakopsora pachyrhizi by delivering dsRNA targeting a gene encoding chitin synthase. Song et al. (2018) targeted a myosin-5 gene in Fusarium asiaticum. Spada et al. (2023) targeted two genes, BcBmp1 encoding a MAP kinase essential for fungal pathogenesis, and BcPls1, encoding a tetraspanin involved in appressorium penetration, in Botrytis cinerea by applying dsRNA onto lettuce leaves.

The RNA molecules of the present invention, particularly those with G:U basepairs in the dsRNA region(s), may be used and are expected to be effective in fission yeast species which are competent for RNAi such as Schizosaccharomyces pombe and Pichia species, but also in budding yeast species such as Saccharomyces castellii, Kluyveromyces polysporus and Candida albicans (Drinnenberg et al., 2009). They may also be used and are expected to be effective in fungal species such as Neurospora crassa, Magnaporthe grisea, Magnaporthe oryzae, ascomycetes such as Aspergillus nidulans, Blumeria graminis, Botrytis cinerea, Colletotrichum species, Cryptococcus neoformans, Coprinus cinereus, Fusarium species, Mycosphaerella graminicola, Phakopsora pachyrhizi, Puccinia species, Ustilago hordei, Verticillium dahliae and Rhizopus oryzae which have RNAi machinery (Drinnenberg et al., 2009). In an embodiment of the present invention, the target RNA molecule is not a RNA transcript from Cyp51 gene of the fungal pathogen Rhizoctonia solani or a RNA transcript from the CesA3 cellulose synthase gene in Phytophthora cinnamomi. In an embodiment, the target RNA molecule is not a RNA transcript from a plant Mlo gene. The common terms for diseases caused by plant pathogenic fungi include anthracnose, leaf spot, rust, wilt, blight such as head blight, coils, scab, gall, canker, damping-off, root rot, mildew, and dieback; the RNA molecules of the present invention can be used to reduce these disease symptoms, preferably comprising the Δ22 modification to produce asymmetric RNA molecules and/or the G:U basepairs as described herein. These diseases include Anthracnose; Botrytis rots; Downy mildews; Fusarium rots; Powdery mildews; Rusts; Rhizoctonia rots; Sclerotinia rots; Sclerotium rots. Others are specific to a particular crop group, e.g. Clubroot caused by Plasmodiophora brassicae in brassicas, Leaf blight caused by Alternaria dauci in carrots, and Red root complex in beans. The RNA molecules may or may not comprise G:U basepairs, and the dsRNA region(s) preferably comprise at least 100 basepairs.

In an embodiment, an RNA molecule of the present invention is directed to the prophylactic or therapeutic treatment of infection by a fungal pathogen selected from the group consisting of: Albugo candida; Alternaria spp.; Armillaria mellae; Arthrobotrys oligosporus; Blumeria graminis (for example by targeting Mlo genes in the host plant), Boletus granulatus; Botritis cinerea; Botrytis fabae; Candida albicans; Claviceps purpurea; Colletotrichum species; Cronartium ribicola; Epicoccum purpurescens; Epidermophyton floccosum; Fomes annosus; Fusarium oxysporum; Gaeumannomyces graminis var. tritici; Glomerella cingulata; Gymnosporangium juniperi-virginianae; Leptosphaeria maculans; Microsporum canis; Monilinia fructicola, Physoderma alfalfae; Phytopthera infestans; Pityrosporum orbiculare (Malassezia furfur); Plasmodiophora brassicae; Polyporus sulphureus; Puccinia spp. such as Puccinia sorghi; Pythium species; Rhizoctonia solani; Sclerotium rolfsii and S. cepivorum; Septoria apiicola; Trichophyton rubrum; T. mentagrophytes; Uromyces appendiculatus; Ustilago species; Venturia inaequalis; and Verticillium dahliae. The RNA molecules of the present invention, preferably those with G:U basepairs in the dsRNA region as described herein, are useful to reduce gene expression through HIGS or SIGS of the fungal pathogens: Aspergillus flavus and Aspergillus parasiticus, especially in cereal plants such as maize and rice, cassava and nuts such as peanuts; species of the genus Colletotrichum that cause anthracnose disease, particularly post-harvest, on legumes such as soybean and fruits such as strawberry, bananas, stone fruit, pears, guava, avocado, mango, citrus, dragon fruit and papaya, including species C. acutatum, C. gloeosporioides and C. truncatum; Cronartium quercuum f. sp. fusiforme that causes white pine blister rust and fusiform rust on pine trees; the obligate biotrophic parasite Erysiphe quercicola which causes powdery mildew in rubber trees; Fusarium culmorum and Fusarium graminearum which cause head blight of wheat, barley and maize; Fusarium oxysporum which inflicts vascular wilt disease in a wide range of economically important crops such as banana, including Fusarium oxysporum f. sp. lycopersici in tomato and Fusarium oxysporum f. sp cubense, the causal agent of Panama disease in banana; Leptosphaeria maculans which causes blackleg disease in canola; Magnaporthe oryzae in cereals such as rice; rust fungi of the order Pucciniales on legumes such as soybean and cereals such as wheat; Sclerotinia sclerotiorum which causes diseases in more than 400 plant species including the important oilseed crop Brassica napus, and Ustilaginoidea virens in cereals such as rice.

Various essential genes of a variety of pathogens are known to the those of skill in the art, for example summarised in WO 93/10251, WO 94/17194.

### Use of RNA Molecules to Provide Resistance to Plants Against Plant-Parasitic Nematodes

Plant-parasitic nematodes (PPN) are a severe threat to crop production and, as estimated in 2013, cause damage to crops estimated to be worth up to USD 173 billion per year (Jones et al., 2013; Elling 2013). As used herein, a “plant parasitic nematode” is an organism of the Phylum Nematoda which feeds on and causes damage to a plant host. PPN are typically root parasites and can be sedentary or migratory and are grouped as ectoparasites or endoparasites. Migratory PPN remain mobile throughout their development whereas the sedentary endoparasites enter the roots as stage-two juveniles, hatched from eggs in the soil, and migrate to the vascular tissues where they induce a large feeding site on which they are totally dependent for the completion of their life cycle. According to Jones et al. (2013), the top 10 nematode genera or species in terms of economic damage they cause are: (1) root-knot nematodes (Meloidogyne spp.) which have a wide host range in many plant species; (2) cyst nematodes (Heterodera spp. or Globodera spp.) (3) root lesion nematodes (Pratylenchus spp.); (4) the burrowing nematode Radopholus similis; (5) Ditylenchus dipsaci; (6) the pine wilt nematode Bursaphelenchus xylophilus; (7) the reniform nematode Rotylenchulus reniformis; (8) Xiphinema index; (9) Nacobbus aberrans; and (10) Aphelenchoides besseyi. Specific nematodes of importance include Globodera rostochiensis (golden nematode) and G. pallida for potato, Meloidogyne chitwoodi in potato, Meloidogyne javanica, Meloidogyne hapla, Meloidogyne arenaria, Meloidogyne enterolobii (guava root-knot nematode) in tomato, Rotylenchulus reniformis in cotton, soybean and pineapple, Pratylenchus coffeae in banana and coffee, Pratylenchus thornei and Pratylenchus zeae in cereals and sugarcane, Pratylenchus vulnus in nut species such as walnut, Pratylenchus penetrans in many plant species including alfalfa (Medicago sativa), bean (Phaseolus vulgaris), corn (Zea mays), potato (Solanum tuberosum) or ornamental crops such as lily (Lilium candidum), boxwood (Buxus sempervirens), fruit trees such as apple (Malus domestica), peach (Prunus persica), or raspberry (Rubus idaeis), Bursaphelenchus xylophilus in pine trees which is carried from tree to tree by the beetle Monochampus alternatus, Xiphinema index for grapevine, Pratylenchus goodeyi and Helicotylenchus multicinctus for banana, and Aphelenchoides besseyi (rice white-tip nematode, RWTN) in rice. Cyst nematodes (Heterodera spp.) of great importance include H. schachtii for sugarbeet, H. glycines for soybean and H. avenae and H. filipievi for cereals.

Numerous reports in the scientific literature describe that conventional RNAi can be used, with varing levels of success, for a range of nematodes to silence genes involved in parasitism, feeding, growth, reproduction, behaviour, resistance to nematicides, etc. The RNA molecules of the present invention can be used to reduce gene expression in these nematodes and are expected to provide greater effects than the corresponding conventional molecules, in particular by in planta expression of the longer, asymmetric RNA molecules having at least 88 nt or at least 100 nt antisense sequences with G:U basepairs and the Δ22 modification, preferably having the Δ22 modification to produce asymmetric RNA molecules and more preferably in the ledRNA structures.

Reduction of target gene activity by conventional RNAi through oral feeding of dsRNA molecules has been demonstrated in PPN, as reviewed by Atkinson et al. (2012), Banerjee et al. (2017), Bharathi et al. (2023), Hewezi and Baum (2015), Ibrahim et al. (2019), Jones and Fosu-Nyarko (2014), Kaur et al. (2021), Lilley et al. (2012), Lim et al. (2020) and Yu et al. (2022). Early reports used soaking of nematodes in solutions of dsRNA, for example Bakhetia et al. (2005) targeted a dual oxidase gene, Chen et al. (2005b) targeted secreted β-1,4, endoglucanases of the potato cyst nematode Globodera rostochiensis, Urwin et al. (2002) targeted genes encoding a cysteine proteinase or a C-type lectin, and Rosso et al. (2005) targeted two genes expressed in the subventral esophageal glands of the nematode, the calreticulin (Mi-crt) and the polygalacturonase (Mi-pg-1) genes in Meloidogyne incognita. Joseph et al. (2012) also delivered dsRNA by soaking to reduce the expression the migratory endoparasitic nematode Pratylenchus coffeae genes pat-10 and unc-87. Soaking the nematodes in a solution containing the RNA molecules, effectively a form of oral delivery, is therefore a suitable means to test an RNA molecule for its ability to reduce a specific gene expression and/or cause a phenotype. Alternatively, the RNA molecules of the present invention may be produced in bacterial or yeast cells such as Saccharomyces cerevisiae and provided to the nematodes.

Many of the reports cited in the reviews used plant-expressed conventional RNA molecules, mostly hairpin RNAs, to reduce nematode gene expression after feeding on the transgenic plants. Ajjappala et al. (2014) targeted a prefoldin-2 gene (Accession no. KM112262) against M. incognita and observed reduced root knot and egg mass numbers compared to the untransformed controls. Banerjee et al. (2018) targeted two cuticle collagen genes, namely Mi-col-1 (Accession no. U40766) and Lemmi-5 (AF006727) involved in the synthesis and maintenance of the cuticle in M. incognita, by expression of hairpin RNA in transgenic tomato plants and observed reduced gall numbers. Baniya et al. (2021) soaked second-stage juveniles of M. incognita with dsRNA targeting two sex-determining genes, sdc-1 (Minc3s00642g15517) and tra-1 (Minc3s00848g18057) and observed downregulation of gene transcripts and reduction in egg production. Calderon-Urrea et al. (2012) targeted a programmed cell death (PCD) regulator gene ced-9, a homolog of C. elegans ced-9 (Accession no. L26545) in M. incognita. Charlton et al. (2010) targeted two genes from M. incognita, a dual oxidase gene (Accession no. DQ082753) implicated in the tyrosine cross-linking of the developing cuticle and a subunit of signal peptidase (spc3, Accession No. BQ613397) which is required for the processing of secreted proteins and observed a reduction in nematode numbers in the roots and retardation of female development. Chaudhary et al. (2019) expressed a hairpin RNA in transgenic eggplant, targeting the Mi-msp-1 effector gene which is expressed in the subventral pharyngeal gland cells of M. incognita and observed delayed development and reproduction. Chukwurah et al. (2019) expressed a hairpin RNA in transgenic tomato plants, targeting a gene (MiPolA1) encoding the largest subunit of RNA polymerase I enzyme and observing reduced nematode reproduction.

Dabrowska-Bronk et al. (2015) expressed a hairpin RNA in transgenic tomato plants, targeting tomato genes encoding a small GTP-binding protein (NGB, Accession No. XP004229516) or an auxin-binding protein (NAB/ERabp1, NP001234826) that are host genes upregulated upon nematode feeding, and observing reduced nematode reproduction of the potato cyst nematode (Globodera rostochiensis). De Souza Junior et al. (2013) expressed dsRNA in transgenic tobacco targeting three protease genes from M. incognita, namely proteases Mi-asp-1 (Accession no. DQ360827), Mi-ser-1 (AY714229) and Mi-cpl-1 (AJ557572) and observed reduced nematode egg production. Ding et al. (2021) transgenically expressed hairpin RNA in the fungus Botrytis cinerea, targeting genes encoding fatty acid and retinol binding protein (FAR) in Aphelenchoides besseyi, and observed reduced reproduction of the nematode. Dinh et al. (2014) expressed hairpin RNAs in transgenic Arabidopsis and potato complementary to a Meloidogyne chitwoodi effector gene Mc16D10L and observed reduced nematode reproduction. Dutta et al. (2015) targeted a M. incognita-specific protease gene, cathepsin L cysteine proteinase (Mi-cpl-1), by expression of hairpin RNA in transgenic tomato plants and observed reduced root knot and egg mass numbers.

Fairbairn et al. (2007) expressed hairpin RNA in transgenic tobacco, targeting a gene encoding a Meloidogyne javanica zinc finger transcription factor, MjTis11 (Accession No. BE578298), and observed reduction of gene expression along with the presence of processed siRNA molecules. Huang et al. (2006) expressed a hairpin RNA targeting a root-knot nematode (RKN) parasitism gene, 16D10 from M. incognita. Expression of the dsRNA in Arabidopsis plants resulted in resistance against four major RKN species. Huang et al. (2014) expressed RNAi molecules in tomato seedlings, targeting a M. incognita gene encoding mitochondrial ATP synthase B subunit (MiASB). Ibrahim et al. (2011) expressed hairpin RNAs in transgenic soybean roots, targeting four genes encoding L-lactate dehydrogenase (LDH; Accession No. AW828669), mitochondrial stress-70 protein precursor (MSP; Accession No. BI773411), ATP synthase beta-chain mitochondrial precursor (Accession Nos. BI773402 and BI773383) or tyrosine phosphatase (TP; Accession No. AW570920) in M. incognita, observing reduced nematode gall formation. Joseph et al. (2012) targeted two genes in Pratylenchus coffeae, namely pat-10 and unc-87. Joshi et al. (2019) expressed dsRNA in transgenic Arabidopsis targeting an oesophageal gland effector gene (msp2). Joshi et al. (2020) expressed dsRNAs in transgenic Arabidopsis, targeting four genes in Meloidogyne javanica, namely msp3 (Accession no. AF531162) which is expressed in the sub-ventral pharyngeal gland, msp5 (AF531164), msp18 (AY134437) and msp24 (AY134443) which are expressed in the dorsal gland, and observed reduction of gene expression and reduced nematode reproduction.

Klink et al. (2009) identified 150 homologs in the soybean cyst nematode Heterodera glycines of genes that have lethal phenotypes when mutated in C. elegans and identified those that were induced during the parasitic stages of infection. Homologs of small ribosomal protein 3a and 4 (Hg-rps-3a, Accession no. CB379877 and Hg-rps-4, CB278739), synaptobrevin (Hg-snb-1, BF014436) and a spliceosomal SR protein (Hg-spk-1, BI451523.1) were tested for functionality by expressing dsRNAs in transgenic roots, observing reduced nematode reproduction. Kohli et al. (2018) targeted a gene encoding a Notch-like receptor protein, GLP-1, that has an essential role in pharyngeal development in M. incognita by expressing a hairpin RNA in transgenic Arabidopsis. Koulagi et al. (2020) engineered resistance against M. incognita and tomato leaf curl virus (ToLCV) by expressing dsRNA constructs targeting an integrase gene of M. incognita together with an AC4 gene of ToLCV, in transgenic tomato. Kumar et al. (2017) targeted two housekeeping genes, splicing factor and integrase, in Meloidogyne incognita in transgenic Arabidopsis. Kumar et al. (2022) targeted two effector genes, Mi-msp10 and Mi-msp23, in M. incognita by expression in transgenic Arabidopsis.

Kyndt et al. (2013) targeted two housekeeping genes, encoding the splicing factor Hs-U2AF and the vacuolar Hs-H⁺ ATPase, and one candidate effector gene, the ubiquitin extension protein Hs-ubi, by expression of hairpin RNA in transgenic Arabidopsis. They observed that host-generated RNAi could suffer from high levels of transcriptional silencing of the transgene, leading to varying expression levels within and between transgenic lines. The present inventors expect that such a transcriptional effect could be mitigated by using RNA molecules of the present invention having C to T substitutions of the sense sequence i.e. providing G:U basepairs in the dsRNA, as exemplified herein.

Other transgenic studies have been reported targeting M. incognita genes through expression of dsRNA in the plants, often as hairpin RNA: targeting three essential genes encoding cysteine protease (Mi-cpl), isocitrate lyase (Mi-icl) or splicing factor (Mi-sf) driven by the pUceS8.3 constitutive soybean promoter in cotton (Lisei-de-Sa et al., 2021), genes encoding a heat-shock protein 90 (HSP90) or isocitrate lyase (ICL) in tobacco (Lourenco-Tessutti et al., 2014), three genes encoding chitin synthase (CS), glucose-6-phosphate isomerase (GPI) or trehalase 1 (TH1) in N. benthamiana (Mani et al., 2020), a gene encoding a secreted effector protein 4 (Mi-SP4) in transgenic rice (Nguyen et al., 2022), a rpn7 gene (Mi-Rpn7) in soybean roots (Niu et al., 2012), genes encoding two FMRF-amide like peptide genes (flp-14 and flp-18) in tobacco (Papolu et al., 2013), concomitant silencing of three M. incognita FLP genes Mi-flp-1 (Accession No. KC517344.1), Mi-flp-12 (AY804187.1) and Mi-flp-18 (AY729022.1) in tuberose plants (Singh et al., 2023), two pharyngeal gland-specific effector genes (msp-18 and msp-20) by expression in eggplant (Shivakumara et al., 2017), genes encoding integrase (Accession No. AW871671) and splicing factor (AW828516) from M. incognita using hairpin RNAs expressed in transgenic tomato from an inducible promoter from Arabidopsis pAt1g74770 (Thorat et al., 2024), and a conserved RKN effector gene (16D10) in transgenic grapevine (Yang et al., 2013).

There are other reports of in planta expression of dsRNA targeting genes in nematodes other than M. incognita: for example targeting genes encoding Cpn-1, Y25 and Prp-17 in Heterodera glycines (Li et al., 2010) in soybean, a gene encoding a Ca2⁺-binding multifunctional protein in Radopholus similis expressed in the oesophageal glands and gonads of females (Li et al., 2015) in tomato, a gene encoding Cathepsin B in Radopholus similis (Li et al., 2015b) in tobacco, a gene encoding a cysteine protease Cathepsin S in Radopholus similis (Li et al., 2017b), genes encoding Actin-4 (act-4, Accession MIC00350) and proteasomal alpha subunit 4 (pas-4, MIC06652) (Roderick et al., 2018), four parasitism genes 3B05 (Accession No. AF469058), 4G06 (AF469060), 8H07 (AF500024) and 10A06 (AF502391) in the sugar beet cyst nematode Heterodera schachtii in transgenic Arabidopsis (Sindhu et al., 2009), two H. glycines genes related to reproduction and fitness HgY25 and HgPrp17 in transgenic soybean (Tian et al., 2019), two locomotion-related genes of Pratylenchus penetrans Pp-pat-10 (AAW56830 in M. incognita) and Pp-unc-87 (AAT70232 in H. glycines) expressed in soybean roots (Viera et al., 2015), and a Pv010 gene (Accession No. CV200529) gene from Pratylenchus vulnus in walnut (Walawage et al., 2013).

Patel et al. (2010) expressed a hairpin RNA in A. thaliana using a Ntcel7 gene promoter, reported to be upregulated in nematode feeding sites, targeting a parasitism gene encoding a Hs4F01 annexin-like effector in the soybean cyst nematode, H. glycines, and observed gene down-regulation and significantly reduced nematode infection levels. Steeves et al. (2006) expressed a hairpin RNA in transgenic soybean plants, targeting a major sperm protein (MSP) gene from H. glycines and observed reduced reproduction of the nematodes that fed on the plants. Yadav et al. (2006) expressed hairpin RNAs targeting genes encoding either integrase (Accession No. AW871671) or splicing factor (Accession No. AW828516) proteins in M. incognita and observed a reduced number of knots on the transgenic plants and abnormal development of the nematodes. Wang et al. (2012) targeted a gene encoding arginine kinase (BxAKI, Accession No. EU853862) in Bursaphelenchus xylophilus, the PPN that causes pine wilt disease, by soaking the nematodes with dsRNA. Wang et al. (2016b) expressed a hairpin RNA in transgenic filamentous fungi against four dumpy genes (Bx-dpy-2, 4, 10 and 11) of Bursaphelenchus xylophilus in order to deliver the RNA to the nematodes.

In the patent literature, U.S. Pat. No. 8,716,554 reported targeting of a collagen gene (Col-5, AF289026) in Meloidogyne javanica through expression of a dsRNA in transgenic tobacco plants. WO2007/104570 describes M. incognita genes designated as Mi05, Mi11, Mi38, Mi40, Mi101, Mi109, Mi111, Mi116, Mi125, Mi127, Mi128 and Mi129 that can be targeted for downregulation by RNAi molecules.

A wide range of target genes can be targeted with the RNA molecules of the present invention. For example, Iqbal et al. (2020) tested dsRNA against 20 different target genes involved various stages of RNAi in M. incognita including rsd-3 (Accession No. CABB01006346), xpo-1 (CABB01004119), xpo-2 (CABB01000462), drh-1 (CABB01006008), drsh-1 (CABB01000477), pash-1 (CABB01004277), vig-1 (CABB01000081), smg-2 (CABB01008394), smg-6 (CABB01000011), ego-1 (CABB01000449), eri-1 (CABB01001883), gfl-1 (CABB01000795), rha-1 (CABB01000079), mes-2 (CABB01002321), mes-6 (CABB010000967), mut-7 (CABB01000055), ekl-4 (CABB01002952), csr-1 (CABB01000355), ppw-2 (CABB01001343) and 2242 (CABB01002242), and observed variability in effectiveness. Eri-1 and gfl-1 encode RNAi inhibitors and are preferred targets in nematodes for the present invention. Viera et al. (2015) identified numerous highly-expressed genes including parasitism genes in Pratylenchus penetrans. Homologous genes can be targeted in other PPN. RNAi inhibitor genes are also ideal target genes, such as PPN genes encoding homologs of C. elegans genes eri-1, xrn-2, adr-2, xrn-1, adr-1, lin-15b, eri-5, eri-6/7 and eri-3. Wubben et al. (2010) identified many expressed genes as potential targets in the transcriptome of the reniform nematode Rotylenchulus reniformis. Other target genes which have been down-regulated in PPN include Mi-msp-9 (Xue et al., 2013), Mi-msp-12 (Xie et al., 2016), Mi-msp-16 (Huang et al., 2006; Yang et al., 2013; Dinh et al., 2014), Mi-msp-18, Mi-msp-20 (Shivakumara et al., 2017) and Mi-msp-40 (Niu et al., 2016), or encode 4G06, 3B05, 8H07, 10A06, 30C02 and Gp-hyp proteins.

Dalzell et al. (2011) identified homologs of the C. elegans RNAi machinery components in a range of PPN. They reported, in summary, that similarities in the RNAi effector complements of nematode parasites support the broad applicability of RNAi in nematodes. After soaking M. incognita nematodes in solutions containing siRNA molecules, Danchin et al. (2013) observed significant and reproducible reduction of M. incognita infestation for 12 of 16 tested genes compared to control nematodes. They had identified and analysed 15,952 nematode genes conserved in genomes of PPN species but absent from genomes of chordates, plants, etc.

Based at least in part on Dalzell et al. (2011), the present inventors expect that each of the above-mentioned genes and their homologs in plant-parasitic nematodes can be effectively targeted with the RNA molecules of the present invention, particularly the longer RNA molecules having at least 100 nt antisense sequences which are hairpin RNA or ledRNA molecules, in particular those having G:U basepairs and/or the Δ22-, Δ23- or A24-modifications, preferably the Δ22 modification, to produce asymmetric RNA molecules. It is expected that these molecules will have increased efficacy in reducing target gene expression compared to the conventional dsRNA molecules, for example when expressed in planta.

It is also noted that the above-mentioned reports did not achieve optimal levels of control of the nematodes, even though reduced nematode reproduction was often observed. Increased efficacy is therefore desired which can be achieved with the RNA molecules of the present invention, particularly by simultaneously targeting two or more genes in the nematodes, for example, 3, 4, 5, 6 or more target genes. All of the reports described above for nematodes used conventional dsRNA molecules, not the RNA molecules of the present invention. The RNA molecules of the invention can be used to reduce target gene expression in PPN, including of all of the target genes referred to in those publications, in particular the hairpin or ledRNA molecules of the present invention having G:U basepairs when expressed in planta, or without G:U basepairs. The RNA molecules optionally have the Δ22, 423 or 424 modification, or combinations thereof, preferably the Δ22 modification to produce asymmetric RNA molecules, and preferably with at least 100 basepairs in the dsRNA region(s). It is expected those molecules will provide enhanced knockdown of the target gene activity in the PPN.

### Target RNA Molecules in Mammalian Animals

The RNA molecules of the present disclosure can be used to decrease expression and/or amount of any target RNA molecule(s) that is associated with a disease in a mammalian subject such as a human subject. For example, the target RNA molecule can be an oncogene or tumour suppressor gene transcript. Exemplary oncogenes include ABL1, BCL1, BCL2, BCL6, CBFA2, CBL, CSF1R, ERBA, ERBB, EBRB2, FGR, FOS, FYN, HRAS, JUN, LCK, LYN, MYB, MYC, NRAS, RET or SRC. Exemplary tumour suppressor genes include BRCA1 or BRCA2; adhesion molecules; cyclin kinases and their inhibitors.

### Production and Delivery of RNA Molecules

There is now a vast literature on the production and delivery of RNA molecules to cells for RNA interference, using a variety of delivery mechanisms from host-induced gene silencing through expression of a gene encoding the RNA molecule, various formulations e.g. nanoparticle formulations. For example, Hough et al., (2022) have reviewed strategies and methods for producing RNA molecules, including in cell free (in vitro) production systems for topical delivery to organisms such as plants, and in bacteria, for example E. coli strain HT115. They also review protection or encapsulation of the RNA molecules. WO2017/176963 discloses methods for cell-free production of double-stranded RNA molecules using heat stable RNA polymerase. WO2019/075167 discloses methods for the production of nucleoside triphosphates and RNA. WO2022/235895 discloses methods for stabilising and formulating RNAi molecules for agricultural applications, including preparation of compositions comprising the RNA molecules, a primary surfactant and a metal-ion sequestrant. All of such compositions and methods can be used with the RNA molecules of the present invention.

Exogenous delivery of dsRNA molecules to plants has been reviewed by Dalakouras et al., (2020). Goodfellow et al., (2019) and Ross et al., (2024) describe delivery to insects and pathogens using bacteria that express the RNA molecules. Arjunan et al., (2024) reviewed using various nanoparticle formulations, as did Palli (2023). Silver et al., (2021) and Flynt (2020) reviewed delivery to insects. Bally et al., (2018) reviewed the production of dsRNA molecules in plant chloroplasts. Qiao et al., (2021) reviewed use of spraying to delivery RNA molecules exogenously. Several studies confirmed the reduction of gene expression in plants by adsorption of dsRNA molecules through different tissues, for example by trunk injection or petiole absorption, or by root soaking in a solution of dsRNAs (Dalakouras et al., 2018).

### Formulations/Compositions

RNA molecules according to the present disclosure can be provided as various compositions or formulations. For example, RNA molecules may be in the form of a solid, ointment, gel, cream, powder, paste, suspension, colloid, foam or aerosol. Solid forms may include dusts, powders, granules, pellets, pills, pastilles, tablets, filled films (including seed coatings) and the like, which may be water-dispersible (“wettable”). In one example, the composition is in the form of a concentrate.

In an embodiment, RNA molecules are provided as a topical formulation. In an example, the formulation stabilises the RNA molecule in formulation and/or in-vivo. For example, RNA molecules may be provided in a lipid formulation. For example, RNA molecules may be provided in liposomes. In an example, the formulation comprises a transfection promoting agent.

The term “transfection promoting agent” as used herein refers to a composition added to the RNA molecule for enhancing the uptake into a cell including, but not limited to, a plant cell, an insect cell or a fungal cell. Any transfection promoting agent known in the art to be suitable for transfecting cells may be used. Non-limiting examples of suitable commercially available transfection reagents include Lipofectamine (Life Technologies) and Lipofectamine 2000 (Life Technologies).

In an embodiment, RNA molecules of the invention are incorporated into formulations suitable for application to a field. In an embodiment, the field comprises plants. Suitable plants include crop plants (for example, cereals and pulses, maize, wheat, potatoes, tapioca, rice, sorghum, soybean millet, cassava, barley, or pea), or legumes. The plants may be grown for production of edible roots, tubers, leaves, stems, flowers or fruit. In an example, the crop plant is a cereal plant. Examples of cereal plants include, but are not limited to, wheat, barley, sorghum oats, and rye. In these examples, the RNA molecule may be formulated for administration to the plant, or to any part of the plant, in any suitable way. For example, the composition may be formulated for administration to the leaves, stem, roots (e.g. hydroponically), fruit, vegetables, grains and/or pulses of the plant. In one example, the RNA molecule is formulated for administration to the leaves of the plant, and is sprayable onto the leaves of the plant.

Depending on the desired formulation, RNA molecules described herein may be formulated with a variety of other agents. Exemplary agents comprise one or more of suspension agents, agglomeration agents, bases, buffers, bittering agents, fragrances, preservatives, propellants, thixotropic agents, anti-freezing agents, and colouring agents.

In other examples, RNA molecule formulations can comprise an insecticide, a pesticide, a fungicide, a herbicide, an antibiotic, an insect repellent, an anti-parasitic agent, an anti-viral agent, or a nematicide.

In another example, RNA molecules can be incorporated into pharmaceutical compositions. Such compositions would typically include an RNA molecule described herein and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, inhalation, transdermal (topical), transmucosal, oral and rectal administration.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. For example, liposomal suspensions can also be used as pharmaceutically acceptable carriers. These can be prepared according to methods known to those skilled in the art, for example, as described in U.S. Pat. No. 4,522,811.

In an embodiment, the RNA molecules are formulated with layered double hydroxides (LDHs) (also known as bioclay), for instance for application to a field and/or plant. Examples of such LDHs are described in WO 2023/039632, WO 2006/066341 and WO 2015/089543.

RNA molecules according to the present disclosure can be provided in a kit or pack. For example, RNA molecules disclosed herein may be packaged in a suitable container with written instructions for producing an above referenced cell or organism or treating a condition.

### Exemplary Conditions to be Treated

RNA molecules according to the present disclosure may be used in methods of treatment for various conditions. In some examples, the present disclosure relates to a method of treating cancer comprising administering an RNA molecule disclosed herein. The term “cancer” refers to or describes the physiological condition in mammals that is typically characterized by unregulated cell growth/proliferation. Examples of cancer include but are not limited to, carcinoma, lymphoma, blastoma, sarcoma, and leukemia or lymphoid malignancies. More particular examples of such cancers include, but are not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. Accordingly, in an example, the present disclosure relates to a method of treating breast, ovarian, colon, prostate, lung, brain, skin, liver, stomach, pancreatic or blood based cancer.

In other examples, a method described herein is used to treat cancers that are linked to mutations in BRCA1, BRCA2, PALB2, OR RAD51B, RAD51C, RAD51D or related genes. In other examples, a method described herein is used to treat cancers that are linked to mutations in genes associated with DNA mismatch repair, such as MSH2, MLH1, PMS2, and related genes. In other examples, a method described herein is used to treat cancers with silenced DNA repair genes, such as BRCA1, MLH1, OR RAD51B, RAD51C, OR RAD51D.

In other examples of the disclosure, a method described herein is used to kill cells with impaired DNA repair processes. For example, cells with impaired DNA repair may aberrantly express a gene involved in DNA repair, DNA synthesis, or homologous recombination. Exemplary genes include XRCC1, ADPRT (PARP-1), ADPRTL2, (PARP-2), POLYMERASE BETA, CTPS, MLH1, MSH2, FANCD2, PMS2, p53, p21, PTEN, RPA, RPA1, RPA2, RPA3, XPD, ERCC1, XPF, MMS19, RAD51, RAD51B, RAD51C, RAD51D, DMC1, XRCCR, XRCC3, BRCA1, BRCA2, PALB2, RAD52, RAD54, RAD50, MREU, NB51, WRN, BLM, KU70, KU80, ATM, ATR CPIK1, CHK2, FANCA, FANCB, FANCC, FANCD1, FANCD2, FANCE, FANCF, FANCG, FANCC, FANCD1, FANCD2, FANCE, FANCF, FANCG, RAD1, and RAD9. In an example, a method described herein is used to kill cells with a mutant tumour suppressor gene. For example, cells can have one or more mutations in BRCA1 or BRCA2.

In other examples of the disclosure, a method described herein is used to treat virally transformed cells. In other examples of the disclosure, a method described herein is used to kill cells transformed with a latent virus. Exemplary latent viruses include CMV, EBV, Herpes simplex virus (type 1 and 2), and Varicella zoster virus. In other examples of the disclosure, a method described herein is used to treat active viral infections due to viruses that give rise to cancer, immunodeficiency, hepatitis, encephalitis, pneumonitis or respiratory illness. Exemplary viruses include parvovirus, poxvirus, herpes virus.

In other examples of the disclosure, a method described herein is used to treat Zika Virus, Colorado Tick Fever (caused by Coltivirus, RNA virus), West Nile Fever (encephalitis, caused by a flavivirus that primarily occurs in the Middle East and Africa), Yellow Fever, Rabies (caused by a number of different strains of neurotropic viruses of the family Rhabdoviridae), viral hepatitis, gastroenteritis (viral)-acute viral gastroenteritis caused by Norwalk and Norwalk-like viruses, rotaviruses, caliciviruses, and astroviruses, poliomyelitis, influenza (flu), caused by orthomyxoviruses that can undergo frequent antigenic variation, measles (rubeola), paramyxoviridae, mumps, respiratory syndromes including viral pneumonia and acute respiratory syndromes including croup caused by a variety of viruses collectively referred to as acute respiratory viruses, and respiratory illness caused by the respiratory syncytial virus.

### EXAMPLES

### Example 1: Materials and Methods

Rearing of Helicoverpa armigera

A colony of cotton bollworm, Helicoverpa armigera (Lepidoptera: Noctuidae), was provided by Dr. J. Daly (Commonwealth Scientific and Industrial Research Organization, Canberra, Australia). The colony was originally established from moths collected from cotton fields in the Namoi Valley, northern NSW, Australia and maintained continuously in the laboratory since the mid-1980s (Apirajkamol et al., 2020). The population size of the colony at each generation exceeded 200 adults. To maintain the colony, a large number of two-day old fertilised eggs were collected from 40 healthy moths and the eggs disinfected by soaking in a 0.002% (v/v) bleach solution for 10 min and then washing with sterile tap water. The eggs were air-dried and placed in a plastic bag to allow hatching. Subsequently, 1st-instar larvae were transferred to 32-well plates for rearing until pupation under optimum conditions at a temperature of 25±1° C. and a relative humidity of 50±10% with light and dark cycles of 14:10 hours to imitate natural day and night conditions. The larvae were fed on an artificial semi-solid diet which was changed every week to prevent stress from insufficient food. Moths that emerged were fed a honey solution, which was checked every two days and refilled as needed.

The semi-solid artificial diet was prepared for rearing the larvae as follows. First, 130 g of soy flour and 700 mL of filtered water were blended with a stick blender. The mixture was then heated in a microwave oven until it boiled (4-5 min). Next, 22 g of agar (Gelita, Catalog A-181017), 1.7 g of sorbic acid (Sigma-Aldrich, Catalog S1626) and 700 mL of filtered water were combined and gently mixed with a spatula. This mixture was also heated for 4-5 min in the microwave oven to boiling. Both mixtures were then separately stirred and reheated before being combined together by blending with a stick blender. Additional dry ingredients of 60 g of wheat germ, 53 g of brewers yeast and 3.3 g of nipagin (Methylparaben, Sigma-Aldrich, Catalog 79721), were added to the final mixture as well as 5 mL of vegetable oil. After the mixture had cooled to 50-60° C., 3.3 g of L-ascorbic acid (Sigma-Aldrich, Catalog A4403) was added and dissolved in the mixture. Filtered water was added to bring the total volume to 1600 mL and the mixture was blended until well-combined. The mixture was poured into 32-well plates at approximately 5 mL per well and left at room temperature for at least an hour to cool, set and dry. The plates with the diet mixture were immediately used or stored at 4° C. for a maximum of two days before use.

After emerging, H. armigera moths were fed on a honey solution. To prepare the honey solution, 40 g of white sugar, 40 g of honey, and 1 g of sorbic acid were weighed into a 1 L bottle. About 300-400 mL of filtered water was added and the mixture heated in a microwave oven for 2 min on a high setting and shaken to dissolve the sugar. Filtered water was added to bring the final volume to 1 L. The mixture was then sterilised by autoclaving for 15 min at 121° C. After the mixture was cooled to 60° C., 2 g of ascorbic acid was dissolved in the mixture. This was stored at 4° C.

H. armigera larvae for use in feeding assays for testing RNA molecules were obtained by collecting newly hatched, 1st instar larvae from the colony.

Rearing of Spodoptera frugiperda

### Artificial Diet Recipe

Two populations of Fall Armyworm (FAW, Spodoptera frugiperda) derived from insects obtained from Queensland, Australia (Apirajkamol et al., 2023) were maintained in the laboratory on an artificial diet medium modified from previous research (Greene et al., 1976), prepared as follows. To make 1200 mL of artificial diet medium, ingredients were combined in premixes as follows. Premix A contained 100 g of navy bean flour and 25 g of soy flour. Premix B contained 18 g of agar and 1.5 g of sorbic acid. Premix C contained 60 g of wheat germ, 30 g of Brewer's dry yeast, 3.3 g of L-ascorbic acid, 10 g of Vanderzant vitamin mixture for insects (Sigma-Aldrich, Catalog V1007), 15 g of casein (Sigma-Aldrich, Catalog C7078) and 3 g of methylparaben. Mixture A was blended into 600 mL water and heated in a microwave oven to boiling point. Mixture B was blended into 500 mL of water and heated for 4 min until boiling to ensure the agar was dissolved. Mixtures A and B were then combined and mixed with a blender and kept at room temperature until the mix had cooled to approximately 60° C. Mixture C and 5 mL of vegetable oil were added to the mixture of A and B, and the combined medium thoroughly mixed with a blender. This medium was distributed into 45-well trays or 32-well trays and allowed to solidify at room temperature. These trays with medium could be used immediately or stored at 4° C. for up to one week before use.

Spodoptera frugiperda Insect Rearing

To maintain the population through the lifecycle, eggs were collected from moth buckets and transferred to a plastic bag or a round tub and allowed to hatch in a culture room at 25±1° C., 50±10% relative humidity, and under a 14 h: 10 h day-night photoperiod. After 1 or 2 days, hatched neonates were transferred with small paint brushes to round tubs or 45-well plastic trays containing the artificial diet medium, with several neonates per well, and maintained in the culture room. Once they developed into 3rd instar larvae, generally after about 1 week, the larvae were transferred to the 32-well trays, one larva per well. The larvae were maintained in the culture room until pupation. Pupae were collected in a tub and sterilised with 0.3% bleach for 5 min followed by several rinses with running tap water. After being rinsed, about 40 pupae were placed on a petri dish without a lid and transferred to a moth bucket covered with a piece of cloth, with a pot of honey solution as a nutrient source. Once the moths had emerged, they mated naturally in the moth bucket and laid eggs after a few days, thus completing the lifecycle.

Hybrid of Spodoptera frugiperda to Overcome Inbreeding Depression

Eggs from two inbred Spodoptera frugiperda lines, Sf20-1 and Sf20-3, started showing dehydration symptoms and many did not hatch, which may have been caused by inbreeding depression. To overcome this, a hybrid line was created from the two lines by mixing 20 pupae from each line as follows: group 1, Sf20-1 females×Sf20-3 males; group 2, Sf20-1 males×Sf20-3 females. Resultant pupae were placed into moth buckets and left to emerge, mate, and lay eggs. Interestingly, only eggs from group 1 hatched normally, with greater than 90% hatching. Therefore, the new hybrid line was named Sf20-X and used for the subsequent rearing and feeding experiments.

### Synthesis of Genetic Constructs

To design a typical ledRNA or hairpin RNA (hpRNA) construct, a region of the target RNA of about 100-1000 nucleotides in length, typically 400-600 nucleotides, was identified. In one embodiment of a ledRNA construct, 5′ half of the sequence and approximately 100-500 nt of the flanking region and similarly 3′ half and 100-500 nt of flanking region were orientated in an antisense orientation relative to a promoter. These sequences were interrupted with a 200-600 nucleotide sense target sequence (FIG. 1A). The 5′ end of the resultant construct was preceded with a promoter such as a T7 or SP6 RNA polymerase promoter and 3′ end engineered to include a restriction enzyme cleavage site to allow for termination of transcription in vitro. HpRNA constructs were made by ligating the region of the target RNA as a sense sequence to the complement of that sequence as an antisense sequence, sense and antisense meaning with respect to the native promoter of the target RNA gene, to drive the expression, with an intervening spacer sequence, thereby forming an interrupted or spaced apart inverted repeat of the target sequence. The spacer sequence which encoded the loop sequence of the hpRNA was or was not related to the sequence of the target transcript and did or did not include one or two intron sequences which are spliced out of the primary transcript in the plant cells (Smith et al., 2000, Helliwell et al., 2001). The order of the elements in the genetic construct was either: promoter-sense sequence-spacer-antisense sequence-termination site/polyadenylation sequence, or promoter-antisense sequence-spacer-sense sequence-termination site/terminator region.

For transcription in cells such as bacterial cells, promoter and terminator sequences were incorporated to facilitate expression as a transgene, for example using an inducible promoter. The double-stranded region and loop sequence lengths can be varied. The constructs were made using standard cloning methods or ordered from commercial service providers.

### Synthesis of RNA

For in vitro synthesis of RNA molecules such as ledRNA, with or without modifications as described herein, DNA of the genetic construct was first linearised by digestion with a restriction enzyme at 3′ end of the RNA coding sequence, purified and quantified. The DNA was then transcribed using an RNA polymerase such as T7 RNA Polymerase or SP6 RNA Polymerase. For ledRNA molecules, the flanking 5′ and 3′ arms annealed to the central target sequence, the molecule thereby comprising a central double-stranded region with a single nick and terminal loops. The central sequence can be orientated in sense or antisense orientation relative to the promoter (FIG. 1A, 1B respectively).

For in vitro synthesis of hpRNA molecules, with or without modifications as described herein, DNA encoding the molecules was digested at a 3′ restriction site using the appropriate restriction enzyme, purified and quantified. RNA synthesis was achieved using RNA polymerase according to the manufacturer's instructions. The hpRNA was resuspended in annealing buffer (25 mM Tris-HCL, pH 8.0, 10 mM MgCl₂) using DEPC-treated water to inactivate any traces of RNase. The yield and integrity of the RNA produced by this method was determined by nano-drop analysis and gel electrophoresis, respectively.

Synthesis of RNA molecules was achieved in bacterial cells by introducing the constructs into E. coli strain HT115. Transformed cell cultures were induced with IPTG (0.4 mM) to express the T7 RNA polymerase, providing for transcription of the RNA constructs. RNA extraction from the bacterial cells and purification was performed essentially as described in Timmons et al. (2001).

For RNA transcription with Cy3 labelling, the ribonucleotide (rNTP) mix contained 10 mM each of ATP, GTP, CTP, 1.625 mM UTP and 8.74 mM Cy3-UTP. The transcription reactions were incubated at 37° C. for 2.5 hr. The transcription reactions (160 μl) were then transferred to Eppendorf tubes, 17.7 μl turbo DNase buffer and 1 μl turbo DNA were added, and the mixtures incubated at 37° C. for 10 minutes to digest the DNA. Then, 17.7 μl Turbo DNAse inactivation solution was added, mixed and incubated at room temperature for 5 min. The mixture was centrifuged for 2 min and the supernatant transferred to a new RNase free Eppendorf tube. Samples of 1.5 μl of each transcription reaction were electrophoresed on gels to test the quality of the RNA product. Generally, one RNA band was observed of 500 bp to 1000 bp in size depending on the construct. The RNA was precipitated by adding 88.5 μl 7.5 M ammonium acetate and 665 μl cold 100% ethanol to each tube. The mixtures were cooled to −20° C. for several hours or overnight, then centrifuged at 4° C. for 30 min. The supernatant was removed carefully and the pellet of RNA washed with 1 ml 70% ethanol, made with nuclease free water, at −20° C. and centrifuged. The pellet was dried and the purified RNA resuspended in 50 μl 1×RNA annealing buffer. The RNA concentration was measured using nanodrop method and the RNA preparations stored at −80° C. until used.

### Construction of Plasmids for Production of RNA Molecules in Plant Cells

Genetic constructs encoding RNA molecules such as ledRNA or hairpin RNA molecules for transient expression in N. benthamiana leaves or in stably transformed plants were made using a modular cloning system with Golden Gate assembly (Peccoud et al., 2011; Weber et al., 2011) or by standard restriction enzyme-ligation methods. For the former, DNA components were commercially synthesised (Thermo Fisher Scientific, ENSA or Twist Bioscience) and first assembled as individual plasmids, each containing the CaMV 35S promoter (internal part number EC51288) and a CaMV polyadenylation signal/transcription terminator (EC41414) for expression in the plant cells, unless otherwise stated. The expression cassettes were assembled in suitable backbone L1-acceptor vectors (EC47772, EC47742, EC47761, EC47781, EC47751) that included standard T-DNA based flanking regions using Type IIS restriction cloning with the BsaI enzyme for SN Golden Gate construction. When desired, SN constructs with the L1 acceptors were then constructed into multi-gene constructs using Type IIS restriction cloning, with the BbsI/BpiI enzyme for SL Golden Gate construction. The SN constructs were assembled onto a suitable SL acceptor plasmid (EC50505) with a kanamycin resistance gene in position one on the SL constructs as a selectable marker gene for plant transformation. All individual genes retained their own CaMV 35S promoter and CaMV 35S polyadenylation signal/transcription terminator (EC41414). The construct to express the silencing suppressor protein P19 and other standard constructs were as described previously (Wood et al., 2009).

### Plant Transformation Protocols

To transform N. tabacum or N. benthamiana plants, plants were grown aseptically in tissue culture as a source of plant material for transformation. The source plants were established from surface sterilised seeds. To do this, seeds were rinsed with 70% ethanol, then surface sterilised with 5% sodium hypochlorite for 10 min with agitation, followed by rinsing in several changes of water. The seeds were then germinated on plates containing MSO medium at 4.43 g/L (PhytoTechnology Laboratories, Catalogue M519) containing 3% sucrose and 0.8% agar, at pH 5.8. Plants were grown in a growth room at 26° C. using a 16/8 hour light/dark photoperiod. After approximately 2 weeks, developing seedlings were transferred and thinned out to 4 seedlings per deep tissue culture plate and cultured on the same medium and growth conditions. About 2 weeks later, single well-established plants were cultured in tissue culture pots. Leaves from 6-week-old plants were used for Agrobacterium-mediated transformation.

Cultures of A. tumefaciens strain LBA4404 containing the genetic constructs in binary vectors were grown at 28° C. in MG/L medium with antibiotics to maintain selection for the genetic constructs. Cultures having an optical density of between 0.25-0.5 at 600 nm were used to inoculate the plant tissues, as follows. Upper leaves from the tissue-culture grown plants were excised and floated on MG/L medium to maintain turgidity until used, and cut into pieces about 1 cm², including leaf midribs. The Agrobacterium culture containing the genetic construct was added to the leaf pieces, ensuring the explants were completely wet, and left for 20-30 mins with occasional shaking to allow the bacteria to bind to the plant cells along the cut edges. The inoculated explants were then lightly blotted on sterile filter paper to remove excess Agrobacteria and transferred adaxial side down to Co-cultivation Medium MS9 without antibiotics. MS9 contained MSO medium with 3% sucrose, 0.8% agar at pH 5.8, sterilised by autoclaving, and the plant hormones IBA at 1 mg/L and IAA at 0.5 mg/L added after the autoclaving and cooling of the MSO-agar medium to 55° C. The inoculated explants were co-cultivated at 26° C. for 48 h in the dark. Following the co-cultivation period, the explants were transferred to shoot regeneration medium containing MS9 and the plant hormones IBA at 1 mg/L and IAA at 0.5 mg/L plus kanamycin at 100 mg/L and Timentin at 150 mg/L, adaxial side up, plating about 10 explants per plate. These were incubated at 26° C. under lighting with a 16/8-hour photoperiod. The explants were transferred to fresh shoot regeneration medium every 2-3 weeks until shoot development occurred. After 6-8 weeks, shoots that had developed to sufficient size were transferred to root initiation medium containing ⅓MSO, 100 mg/L kanamycin, 150 mg/L Timentin and 1 mg/L IBA. Once individual plants had developed strong roots, small leaf samples were harvested for DNA extraction and testing by PCR for the presence of the selectable marker gene and the desired transgenes. Confirmed transgenic plants were then planted in soil and grown in a glasshouse, allowing the plants to acclimatise gradually.

Arabidopsis thaliana plants were transformed by a standard floral dip method (Clough and Bent, 1998).

### Expression of Genes in Plant Cells in a Transient Expression System

Genes were expressed in plant cells using a transient expression system essentially as described by Wood et al. (2009), with various modifications as outlined below. Nicotiana benthamiana plants were grown in a growth chamber at 23° C. under a 16:8 h light:dark cycle with 90 μmol/min light intensity provided by cool white fluorescent lamps. Binary vectors containing the genes to be expressed in plant cells by a strong, constitutive 35S promoter or the enhanced 35S promoter (e35S; Kay et al., 1987) were introduced into Agrobacterium tumefaciens strain GV3101. A chimeric binary vector, 35S: CMV-2b, for expression of the 2b viral silencing suppressor was separately introduced into A. tumefaciens strain AGL1, as described in WO2010/057246. This viral silencing suppressor was routinely included in the method to maintain gene expression of transgenes introduced together with it. The recombinant A. tumefaciens cells were grown to stationary phase at 28° C. in LB broth supplemented with 50 mg/L carbenicillin or 50 mg/L kanamycin, according to the selectable marker gene on the vector, and 50 mg/L rifampicin. Acetosyringone was added to the culture to a final concentration of 100 μM and the culture then incubated at 28° C. with shaking for another 2.5 hr. The bacteria were then pelleted by centrifugation at 5000×g for 10 min at room temperature. The supernatant was discarded and the pellet was resuspended in a solution containing 10 mM MES pH 5.7, 10 mM MgCl₂ and 100 μM acetosyringone after which the OD600 was measured. A volume of each culture, including the culture containing the viral suppressor construct 35S::CMV-2b, required to reach a final concentration of OD600=0.10 was added to a fresh tube. The final volume was made up with the infiltration buffer. Using a needle-less syringe, leaves were then infiltrated with the culture mixture and the plants were typically grown for a further three to five days after infiltration before leaf samples were recovered for analysis. A control infiltration was typically included which had only the viral suppressor construct 35S:CMV-2b.

For over-expression of more than one gene of interest in combination, each additional gene was introduced separately into an A. tumefaciens strain and grown as described above. Bacterial suspensions were mixed so that each bacterial strain was at a final concentration of OD600=0.10. The bacterial strain containing the gene encoding the viral silencing suppressor 35S::CMV-2b was included in all mixtures at the same concentration. For example, to express four genes in a transient leaf assay and including the viral suppressor construct, the final OD600 of the infiltrated mixture was 5×0.10=0.50 units. The simultaneous over-expression of at least five genes each from separate T-DNA vectors within plant cells in the transient assay format has previously been demonstrated using N. benthamiana (Wood et al., 2009).

RNA Extraction, cDNA Synthesis and Analysis

In order to extract RNA from plant leaf samples such as those which had been infiltrated with Agrobacterium, leaf pieces of about 2×2 cm in area were frozen with liquid nitrogen, ground to a powder, and 500 μl of Trizol reagent (Thermo Fisher Scientific) added per sample. Following this, the Trizol supplier's instructions are followed except with these modifications: the chloroform extraction was repeated and the RNA was dissolved at 37° C. The extracted RNA was treated with RQ1 DNAse (Promega) to remove any extracted DNA. The RNA preparations were then further purified using Plant RNeasy columns (Qiagen). When performed, cDNA synthesis was carried out using Superscript III reverse transcriptase (Thermo Fisher Scientific) according to the supplier's protocol with an oligo-dT primer or with a sequence-specific 3′ oligonucleotide primer. For RT-PCR analysis of each RNA sample, three separate cDNA synthesis reactions were carried out. The 20 μl cDNA reactions were diluted 20-fold in nuclease free water. qRT-PCR was carried out on a Qiagen rotor gene Q real-time PCR machine. 9.6 μl of each cDNA was added to 10 μl of 2× sensifast no ROX SYBR Taq (Bioline) and 0.4 μl of forward and reverse primers at 10 μmol each, for a final reaction volume of 20 μl. All qPCR reactions, for both reference and specific genes, were carried out in triplicate under the following cycling conditions unless otherwise stated: 1 cycle of 95° C./5 min, 45 cycles of 95° C./15 sec, 60° C./15 sec and 72° C./20 sec. Fluorescence was measured at the 72° C. step. A 55° C. to 99° C. melting cycle was then carried out. Control amplifications for a constitutively expressed N. benthamiana GAPDH mRNA were used to normalise gene expression using the comparative quantitation program in the rotor gene software package. The values for each set of three cDNAs, representing the average of triplicate assays, were averaged, allowing for a calculation of the standard error of the mean (SEM).

### Northern Blot Analysis of Longer RNA Molecules

Total RNA was isolated from plant samples using Trizol reagent according to the manufacturer's instructions. The isolated RNA was dissolved in RNase free water and quantified using a nanodrop instrument. Samples of the RNA were loaded onto a formaldehyde-agarose gel for electrophoresis. The gel medium was made by mixing 1.3 g agarose in 95 ml MOPS buffer made in distilled, RNase free water, heating the mixture in a microwave oven to melt the agarose, cooling the mixture and adding 5 ml formaldehyde. The gel was cast in a fume hood, removing any bubbles with a pipette tip and allowed to set. RNA samples of 10-20 μg were denatured before loading on the gel by heating the RNA at 100° C. in the presence of 50% formamide, 17.5% formaldehyde in MOPS buffer. Ethidium bromide or redsafe was added as a stain for the RNA, and the samples loaded immediately onto the gel. Electrophoresis was carried out for 2-3 hr using MOPS buffer as the running buffer, after which the gel was photographed. The gel was blotted overnight to transfer the RNA onto a Hybond-N membrane using 20×SSC as the transfer medium. The transferred RNA was crosslinked to the membrane by exposure to UV light. Pre-hybridisation and hybridisation was carried out at 55° C. in a medium containing 25% formamide (v/v), 0.1% SDS (w/v), 5×SSPE buffer, 5×Denhardt's solution. A ³²P-UTP labelled RNA probe, made by in vitro transcription using T3, T7 or SP6 RNA Polymerase, was added after 2-3 hr pre-hybridisation. Hybridisation was continued at 55° C. overnight. The blot was washed at 65° C. in 2×SSC/0.2% SDS to remove non-specific bound probe, and autoradiographed to detect specifically bound probe.

### Detection of Small RNAs by Northern Blot

Small RNAs (sRNAs) of less than 50 nucleotides length were analysed by Northern blot as follows. Total RNA samples from plants were separated on 17% polyacrylamide/bisacrylamide (19:1) gels containing 42% (w/v) urea in TBE buffer. Samples containing 20-40 μg RNA were denatured prior to electrophoresis by heating the RNA in the presence of 50% deionised formamide containing bromphenol blue stain and XCFF, heating at 95° C. for 2 min, then loading the samples immediately onto the gel. Electrophoresis was carried out at 25-30 mAmp using 1×TBE as the running buffer until the blue dye reached the bottom of the gel, approximately 2 hr. The RNA was transferred to a Hybond-N membrane by electroblotting at 30 V for 1-1.5 hr using 0.5×TBE as the transfer buffer. The RNA was crosslinked to the membrane with UV light. Hybridization was carried out at 42° C. in a medium containing 50% formamide, 250 mM NaCl, 7% SDS in a 125 mM sodium phosphate buffer, pH 7.2. Alternatively, the hybridisation was carried out at 42° C. in a medium containing 50% formamide, 1% SDS and 5×Denhardt's solution in a 5×SSPE buffer, pH 7.4.

RNA probes were prepared by in vitro synthesis using T7 RNA Polymerase and a ³²P-UTP labelled nucleotide with unlabelled ATP, CTP and GTP. After synthesis, DNA was removed by treatment with DNase and the RNA precipitated with ethanol in the presence of ammonium acetate. The average size of the RNA probe was reduced to about 50 nucleotides by treatment with sodium carbonate/bicarbonate at 60° C. before adding to the hybridisation mixture. After hybridisation, the blot was washed twice for 30 min each time in 2×SSC/0.2% SDS at 42° C. If the non-specific background of radioactivity was still too high, the blot was treated with RNase using 2 μg/ml RNase in 2×SSC for 5-10 min at room temperature. The membrane was then autoradiographed overnight against a phosphor screen.

### Yeast Transformation and Culture Conditions

Transformation of yeast strains was performed using the Yeast Transformation Kit (Sigma Aldrich) according to the manufacturer's protocol. For vectors having a Ura gene as selectable marker, transformed colonies were selected by plating the transformation mixture onto minimal medium without uracil (SCMM-U) agar plates, which contained 6.7 g/L yeast nitrogen base, 1.92 g/L synthetic dropout medium without uracil (Sigma Aldrich), 20 g/L glucose, and 20 g/L agar. After 2-3 days of incubation at 30° C., single colonies were re-streaked onto fresh SCMM-U agar plates. The presence of the genetic construct was confirmed by PCR using gene specific primers. A single colony that contained the genetic construct was inoculated into SCMM-U liquid media (containing the same components as SCMM-U agar but without the agar), grown at 30° C. with shaking for 2 days. Glycerol was added to a final concentration of 20% and aliquots stored in −80° C. until further use.

For expression of the genes contained in the genetic construct, an inoculant from the glycerol stock was grown in SCMMM-U liquid media at 30° C. with shaking for 2 days. The cells were collected from the culture by centrifugation and resuspended in SCMM-U induction medium which was identical to SCMM-U liquid media except that the glucose was replaced with 20 g/L galactose, to a final OD600 of 0.4. The culture for induction was grown at 30° C. with shaking for 2 days and the yeast cells were collected by centrifugation for protein extraction and Western blot analysis.

### Isolation of Plant Nuclei and Run-on Transcription Assay

Plant cell nuclei were isolated essentially as described by Meng and Lemaux (2003). Briefly, 1-5 g of leaf tissue was frozen with liquid nitrogen and ground to a fine powder with a cold mortar and pestle. The powder was suspended in 8-40 ml of ice-cold buffer containing 10 mM Tris-HCl, pH 7.6, 1.14 M sucrose, 5 mM MgCl₂ and 7 mM β-mercaptoethanol (H buffer) at a rate of 8 ml/g tissue. The slurry is gently stirred into a low-viscosity liquid and filtered through 2 layers of Miracloth that had been pre-wet with H buffer, keeping the mixture cold. The filtrate was centrifuged at 1000 g for 10 min at 4° C. and the supernatant discarded. The pellet was resuspended in 5-25 ml of cold lysis buffer containing 0.15% Triton X-100 in H buffer, using 5 ml/g tissue. The mixture was centrifuged at 1000 g for 10 min, and the resuspension and centrifugation steps repeated once. The resultant nuclei preparation could be used in run-on transcription assays and/or RNA extracted by resuspending the nuclei in 1 ml Trizol reagent and following the instructions for isolation of RNA.

Isolation and Analysis of Nuclei from Leaf Samples

To isolate cellular nuclei from leaf samples, for example 3-5 days after infiltration with Agrobacterium containing one or more genetic constructs for expression, the protocol of Meng and Lemaux, 2003, was followed. Leaf samples of 1-5 g fresh weight were frozen in liquid nitrogen and ground to a powder using a cold mortar and pestle. Keeping the samples cold, the powder was suspended in ice-cold H buffer which contained 10 mM Tris-HCl, pH 7.6, 1.14 M sucrose, 5 mM MgCl₂ and 7 mM β-mercaptoethanol, using 8 ml of H buffer per gram of leaf tissue (Meng and Lemaux, 2003). The mixture was gently stirred into a low viscosity mixture and filtered through two layers of Miracloth (Calbiochem, La Jolla, CA) pre-wet with H buffer. The filtered mixture was centrifuged at 1000 g for 10 min at 4° C., to pellet the cellular material. The supernatant was discarded. The cell pellet was completely resuspended in cold lysis buffer, which was H buffer containing 0/15% (v/v) Triton X-100, using 5 ml per gram leaf tissue. The mixture was centrifuged 1000 g for 10 min at 4° C. The treatment with cold lysis buffer followed by centrifugation was repeated once. The resultant pellet contained the cellular nuclei, from which RNA was extracted.

### Example 2: Design and Testing in Plants of Hairpin RNAs Comprising G:U Basepairs

### Design of Modified Hairpin RNA Containing G:U Basepairs Targeting GUS RNA

The inventors designed, produced and tested a modified hairpin RNA containing G:U basepairs for its expression level (accumulation level), processing by RNase, and its ability to reduce the expression of a gene encoding the enzyme β-glucuronidase (GUS) as an exemplary target gene in plant cells. This experiment used a gene-delivered approach to provide the hairpin RNAs to the cells and compared a modified G:U hairpin RNA (hpGUS[G:U]) to a corresponding, conventional hairpin RNA (hpGUS[Con]). WO2020/024019 describes the design and synthesis of the constructs encoding either the G:U modified hairpin or the conventional hairpin RNA used as the control in the experiment. Both RNA molecules had a double-stranded region of 200 contiguous basepairs in length, and both targeted nucleotides 804-1003 of the protein coding region of a wild-type GUS sequence (SEQ ID NO: 1). All of the basepairs in the dsRNA region of hpGUS[Con] were canonical basepairs, i.e. G:C and A:U basepairs without any G:U basepairs, and without any non-basepaired nucleotides (mismatches) in the double-stranded region. The modified hairpin RNA hpGUS[G:U] targeted the same 200 nt region (SEQ ID NO: 2) of the GUS mRNA molecule as hpGUS[Con], but included 52 G:U basepairs, formed by substituting all cytidines (C) in the sense sequence of the genetic construct to thymidines (T) (SEQ ID NO: 3) to result in uridines (U) in the sense sequence of the modified hairpin RNA i.e. 26% of the nucleotides of the double-stranded region were involved in G:U basepairs. Thereby, the two hairpin molecules had the same 200 nucleotide antisense sequence that was fully complementary to the GUS target region, and the molecules had a nearly identical loop sequence. In the genetic constructs encoding the hairpin RNAs, the sense and antisense sequences were separated by a spacer sequence included a PDK intron (Helliwell and Waterhouse, 2005; Smith et al., 2000), providing for an RNA loop of about 40 nucleotides in length after splicing of the intron from the primary transcript. The two RNA molecules thereby differed essentially in the presence of G:U basepairs in the modified molecule and G:C basepairs in the corresponding positions in the control molecule.

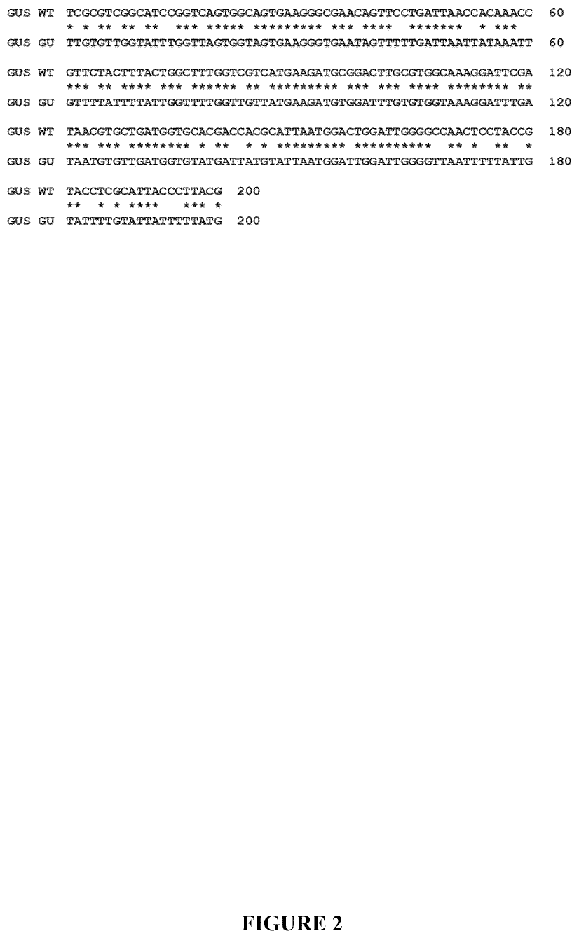

FIG. 2 shows an alignment of the control GUS sense sequence (WT; SEQ ID NO: 2) and the modified sense sequence (GU; SEQ ID NO: 3), showing the positions of the C to T substitutions spread quite evenly across the full length of the 200-nucleotide sense sequence. After transcription of the hpGUS[Con] expression cassette and splicing of the PDK intron from the primary transcript, the hairpin RNA structure prior to any processing by Dicer or other RNases was predicted to have a loop structure of 39 nucleotides. The nucleotide sequence of the hpGUS[Con] stem-loop structure is provided as SEQ ID NO: 4, and its free energy of folding was predicted to be −471.73 kcal/mol. This was therefore an energetically very stable hairpin structure. The free energy was calculated using RNAfold (http://rna.tbi.univie.ac.at/cgi-bin/RNAWebSuite/RNAfold.cgi) based on the nucleotide sequence after the splicing out of the PDK intron sequence. Upon transcription, the hpGUS[G:U] hairpin RNA self-anneals by hybridisation of the 200 nt sense and antisense sequences to form the dsRNA region. The longest stretch of contiguous canonical basepairing in the double-stranded region of hpGUS[G:U] was 10 basepairs. The nucleotide sequence of the G:U hairpin structure including its loop is provided as SEQ ID NO: 5, and its free energy of folding was predicted to be −331.73 kcal/mol, so again energetically very stable.

When transcribed from the expression cassette having the CaMV 35S promoter and 3′ ocs terminator, the hairpin RNAs were embedded in a larger RNA molecule with 8 nucleotides added to the 5′ end and approximately 178 nucleotides added at the 3′ end, without considering addition of any poly-A tail at 3′ end. Since the same promoter-terminator design was used for the modified hairpin RNA, both molecules had these extensions at 5′ and 3′ ends. The length of the hairpin RNA molecules after splicing of the PDK intron was therefore approximately 630 nucleotides.

### Testing the Modified Hairpin RNAs in Transgenic Plants

Plants of the species Nicotiana tabacum (tobacco) transformed with a GUS target gene (WO2020/024019) were used to test the efficacy of the G:U modified and control hairpin RNA constructs. The two hairpin RNA constructs were used to transform PPGH11 and PPGH24 plants already transformed with the GUS target gene to add the hairpin RNA constructs using the Agrobacterium-mediated leaf-disk method as described in Example 1, using 50 mg/L kanamycin as the selective agent. Regenerated transgenic plants containing the T-DNAs from the hpGUS constructs were transferred to soil for growth in the greenhouse and maintained for about 4 weeks before assaying for GUS activity. When assayed, the doubly-transformed transgenic plants were healthy and actively growing and in appearance were identical to non-transformed control plants and the parental PPGH11 and PPGH24 plants. In total, 59 transgenic plants were obtained that were transformed with the T-DNA encoding hpGUS[Con] and 74 plants were obtained that were transformed with the T-DNA encoding hpGUS[G:U]. GUS expression levels in leaf samples were measured using the fluorimetric 4-methylumbelliferyl β-D-glucuronide (MUG) assay (Jefferson et al., 1987) following the modified kinetic method described in Chen et al. (2005). Plants were assayed by taking leaf samples of about 1 cm diameter from three different leaves on each plant, choosing leaves which were well expanded, healthy and green. Care was taken that the test plants were at the same stage of growth and development as the control plants. Each assay used 5 μg protein extracted per leaf sample and measured the rate of cleavage of MUG as described in Chen et al. (2005).

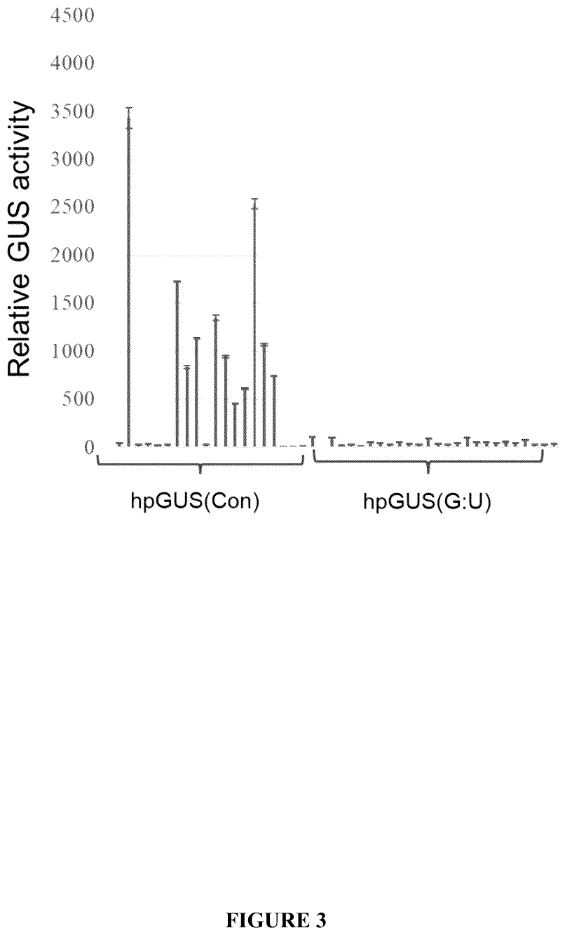

Representative data are shown in FIG. 3, showing GUS activity (MUG units in the assay) for each independent transgenic plant. The genetic construct encoding the canonically basepaired hpGUS[Con] induced strong GUS silencing in many plants, with strong silencing defined in this context as having less than 10% activity level of the parental control plants i.e. at least 90% reduction in activity. Thirty-two of the 59 transgenic plants tested (54.2%) were strongly silenced for the GUS target gene. The other 27 plants all showed reduced GUS activity but retained more than 10% of the enzyme activity relative to the control plants, and so were considered to exhibit weak silencing in this context. The transgenic plants with this construct showed a wide range in the extent of GUS gene silencing (FIG. 3), from less than 1% to about 80% activity remaining, which was typical for conventional hairpin designs (Smith et al., 2000).

In clear contrast, the hpGUS[G:U] construct induced consistent and uniform silencing across the independent transgenic lines, with 71 of the 74 plants (95.9%) that were tested showing strong GUS silencing. When only the strongly silenced lines (<10% remaining activity) were used for comparison and average GUS activities calculated, the hpGUS[Con] plants had slightly greater extent of silencing than the hpGUS[G:U] plants. To test whether the silencing phenotypes would persist in progeny plants, representative transgenic plants containing both the target GUS gene, which was homozygous, and the hpGUS transgene (hemizygous) were self-fertilised. Kanamycin-resistant progeny plants from the hpGUS lines were selected, so discarding any null segregants lacking the hpGUS transgenes. This ensured that the hpGUS transgenes were present in all of the progeny, in either the homozygous or heterozygous state. The progeny plants were assayed for GUS activity. Progeny containing the hpGUS[Con] transgenes obviously fell into two categories, namely those that had strong GUS silencing and others that showed weak or no silencing. These classes correlated well with the phenotype of the previous generation, showing that the extent of target gene silencing was heritable. All of the plants in the hpGUS[G:U] lines tested consistently showed strong silencing. The inventors concluded that the phenotypes observed in the parental generation were generally maintained in the progeny plants.

### Northern Blot Hybridisation Experiments on Transformed Plants

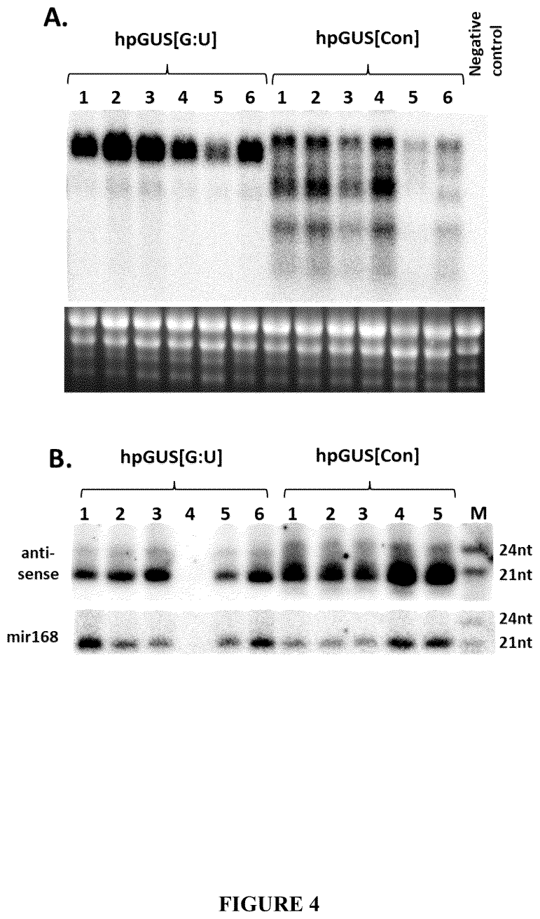

RNA was extracted from leaf samples from six tobacco plants independently transformed with the 35S-hpGUS[G:U] construct and six other plants transformed with the 35S-hpGUS[Con] construct. Samples of 10 μg of each RNA were electrophoresed on an agarose gel, blotted to a membrane and hybridised with a 200 nucleotide GUS sense probe under stringent hybridisation conditions as described in Example 1, to detect RNA molecules having a GUS antisense sequence as used in the dsRNA region of the hairpin RNAs. An autoradiogram of the Northern blot is shown in FIG. 4 (A). The autoradiogram (uppermost panel) showed that the hpGUS[G:U] plants accumulated an intense band for RNA of about the size expected for a full-length hairpin molecule. In contrast, the hpGUS[Con] plants accumulated less full-length RNA molecules but also more smaller RNA molecules, indicating some RNA processing or instability. There was no hybridising signal for the control RNA from a parental plant lacking the hairpin constructs. The photograph of the gel of total RNA used as loading control showed that about equal amounts of RNA were loaded in each lane. It was concluded that the hpGUS[G:U] plants accumulated more long hpRNA molecules than the fully-canonically basepaired hpGUS[Con] plants.

To determine whether the hpGUS[G:U] RNA was processed by one or more Dicers in the transgenic plants and whether sRNAs accumulated, a Northern blot hybridisation was carried out on RNA preparations to detect short RNAs of about 21-24 nucleotides in length, as described in Example 1. The plants used in this experiment also containing the GUS target gene which was expressed as a sense mRNA. The RNA samples were isolated using the hot-phenol method (Wang et al., 2008) and Northern blot hybridization was performed according to Wang et al. (2008) with gel electrophoresis of the RNA samples carried out under denaturing conditions. Five or six plants for each construct were selected for sRNA analysis. The autoradiogram is shown in FIG. 4 (B). The probe used was ³²P-labelled RNA that hybridised to the antisense SRNA sequences.

It was observed that the hpGUS[G:U] plants accumulated less sRNAs than the hpGUS[Con] plants, which was consistent with the increased accumulation of long RNA molecules observed in FIG. 4, panel A. At the same time, the observation that the plants expressing hpGUS[G:U] accumulated lower levels of sRNAs than the strongly silenced plants expressing hpGUS[Con], and yet still showed strong silencing of the target gene, suggested to the inventors that enough of the hpGUS[G:U] hairpin was being processed in the plants to sRNAs to be as effective as the hpGUS[Con] construct.

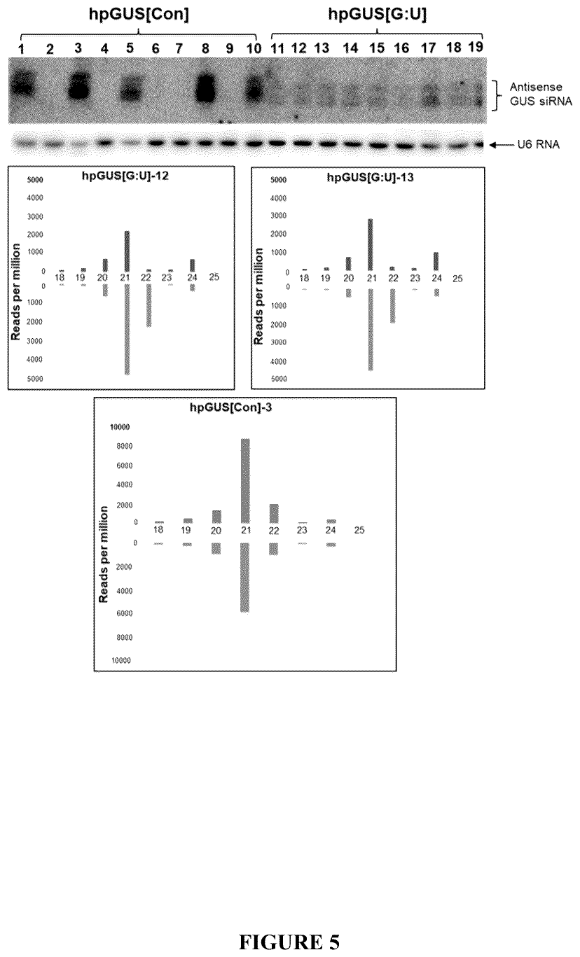

To further investigate this, the small RNA populations in the preparations from ten hpGUS[G:U] and nine hpGUS[Con] transformed plants were analysed by deep sequencing of all linker-amplifiable sRNAs isolated from the plants. The frequency of sRNAs which mapped to the double-stranded regions of the hairpin RNAs was determined. The length distribution of such sRNAs was also determined. The results (FIG. 5) showed that there was an increase in the frequency of 22-mer antisense RNAs calculated as the number of such antisense RNAs per million reads from the hpGUS[G:U] construct relative to the hpGUS[Con] construct. That is, the increase in the proportion of sRNAs of 22 nucleotides (22-mers) in length relative to 21-mers, possibly indicating a shift to more processing of the hpGUS[G:U] hairpin by Dicer-like 2 (DCL2) relative to hpGUS[Con]. Such an increase in the frequency of 22-mers relative to 21-mers was not seen in another experiment for a hairpin RNA targeting an endogenous gene in A. thaliana, so other factors were thought to also play a role.

An important conclusion from these experiments was that the hpGUS[G:U] plants accumulated more longer transcripts of about full-length size than the corresponding hpGUS[Con] plants. At the same time, some of the G:U hairpin RNA molecules were processed by one or more Dicer enzymes to produce sRNAs, enough to produce strong and consistent target gene silencing. This therefore included the production of antisense sRNAs which are thought to be mediators of RNA interference in the presence of various proteins such as Argonaute. The observed production of antisense sRNAs by Northern blot hybridisation implied that the sense sRNAs were also produced from the hpGUS[G:U] RNA, even though these were not observed on the Northern blot. The antisense RNA probe used to detect sense sRNAs had a wild-type GUS sequence so had relatively weak complementarity with the sense sRNAs from hpGUS[G:U], which likely explained the observation for lack of hybridisation. Sense sRNAs with C-to-U substitutions were readily detected by sRNA deep sequencing from the hpGUS[G:U] plants (FIG. 5). It was also concluded that there were clear differences between the hpGUS[Con] and hpGUS[G:U] RNA molecules in the extent and type of processing by Dicers.

### Example 3: Expression of Hairpin RNA from a Pol III Promoter

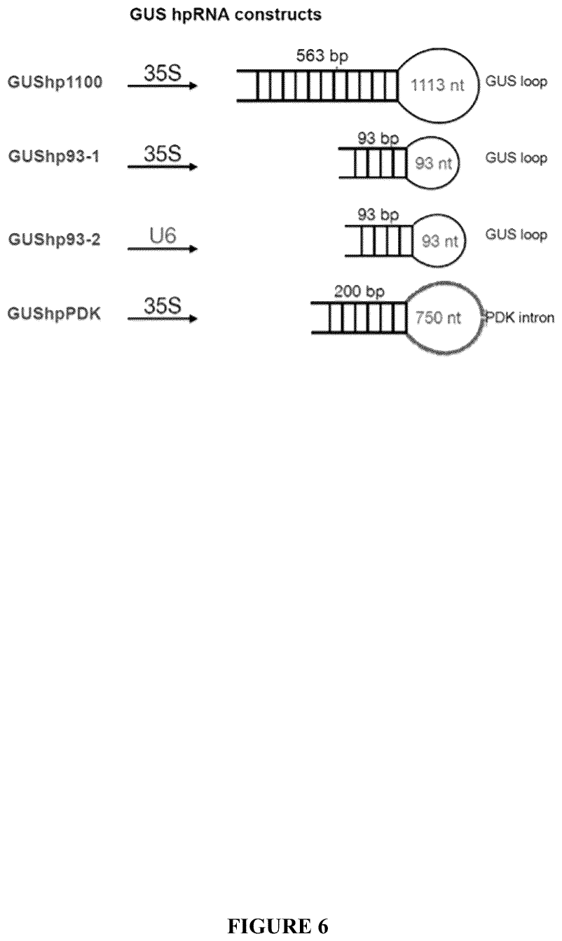

A genetic construct was made which encoded a shorter hpRNA targeting the same GUS target mRNA, having 93-nucleotide sense and antisense sequences for the dsRNA region and a 95-nucleotide spacer to provide the loop sequence, expressed from a 35S promoter (GUShp93-1, FIG. 6). A second, analogous construct was made having an Arabidopsis thaliana U6 promoter, which is a Pol III promoter, rather than the Pol II 35S promoter for expression of the hpRNA. The second construct was designated GUShp93-2 (FIG. 6). This was to compare the expression and processing of the hairpin RNAs when expressed from a Pol III promoter rather than the Pol II promoter. The nucleotide sequence that encodes the stem-loop structure of hairpin RNAs hpGUS93-1 and hpGUS93-2 is provided as SEQ ID NO: 6. A third GUS hpRNA construct was made which included a PDK intron as spacer sequence (GUShpPDK in FIG. 6). These constructs were introduced separately into Nicotiana benthamiana leaf cells for transient expression of the hairpin RNAs and were also used to transform A. thaliana plants for expression as stably integrated and heritable transgenes. When introduced and expressed transiently in the N. benthamiana plants, these constructs all generated distinct RNA fragments of the expected size for the loop sequences. Northern blot hybridisation analysis of GUShp93-1, GUShp93-2 and GUShpPDK-infiltrated N. benthamiana RNA samples all detected RNA molecules of a size corresponding to the length of the loop sequences. Treatment of the RNAs with RNAse R before the gel electrophoresis step, which is an exonuclease specific for 3′ ends of RNA molecules, showed that the loop sequences did not have an open 3′ end and were deduced to have been converted to circular RNAs. The U6 promoter-directed GUShp93-2 construct yielded more loop fragment than the 35S promoter driven GUShp93-1 construct, indicating that the U6 promoter had stronger transcriptional activity than the 35S promoter in the N. benthamiana leaf cells. The possibility that the Pol III-expressed molecules were more stable than the Pol II-expressed molecules could not be excluded.

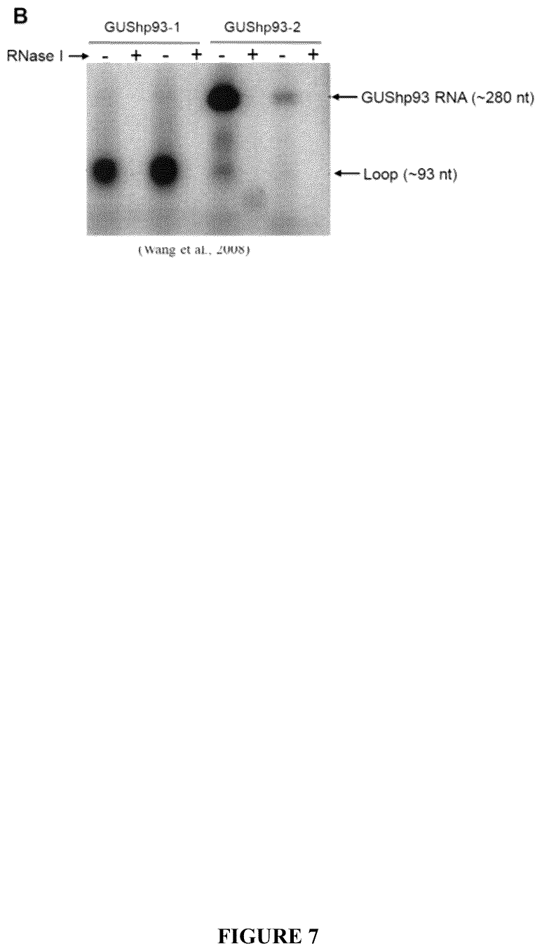

A clear difference in RNA processing from the two constructs was observed, however, in the transgenic A. thaliana plants compared to the same pair of constructs transiently expressed in N. benthamiana. The A. thaliana plants transformed with the GUShp93-2 construct showed accumulation of a full-length hairpin RNA (FIG. 7) compared to plants transformed with the GUShp93-1 construct. Plants with the Pol II-expressed construct accumulated the loop sequence, similar to that observed in N. benthamiana. This was consistent with a reduced extent of silencing of the GUS target gene in the transgenic A. thaliana and tobacco plants compared to the 35S-driven constructs. The inventors concluded that use of the Pol III promoter, for example the Arabidopsis U6 promoter, rather than the Pol II 35S promoter, increased the accumulation of the full-length hpRNA transcripts having only canonical basepairing.

For expression of either G:U hairpin RNA or conventional hairpins with only canonical basepairing in monocotyledonous plants, a monocot Pol III promoter is desirable. For example, a rice U3 gene promoter provides strong expression of G:U and conventional hairpin RNAs in monocot plant cells, such as for example rice, for accumulating high amounts of hairpin RNA.

Following Examples herein demonstrate the design and production of G:U-modified hairpin RNA molecules and asymmetric RNA molecules for reducing expression of target RNA molecules in plants, insects and fungi. Such molecules can also be expressed from Pol III promoters while avoiding premature termination of transcription caused by a run of at least four consecutive T nucleotides. This can be achieved by using some T to C and A to G substitutions in both the sense and antisense sequences, as needed. For example, a sequence such as TTTTT can be modified to TCTCT, and the complementary sequence to AGAGA, etc. This allows for expression of longer target sequences from the Pol III promoter while avoiding premature termination of transcription.

### Example 4: Expression and Processing in Plant and Yeast Cells of hpRNA[G:U] Molecules Targeting a Lepidopteran Insect Gene

### Introduction

Double-stranded RNA (dsRNA)-induced gene silencing, or RNA interference (RNAi), is a promising approach for insect pest control (Zhu and Palli, 2020). Expression in plants of transgenes producing dsRNA which target insect gene transcripts has been shown to confer a level of resistance, albeit weak or partial, against a number of insect pests. For example, dsRNA has been used to target genes in Western Corn Rootworm (WCR; Baum et al., 2007), cotton bollworm (Mao et al., 2011; Jin et al., 2015) and Colorado potato beetle (Zhang et al., 2015b). A potential advantage of the RNAi approach is that it can be readily tailored to target a specific or a selected group of insect pests by choosing appropriate target gene sequences, leaving non-target insects unaffected. One approach is to express a transgene encoding a hpRNA from the chloroplast genome rather than the nuclear genome, since chloroplasts lack Dicer and transcriptional gene silencing. This has been shown to give higher levels of hpRNA accumulation and better control of Colorado potato beetle, a Coleopteran pest (Zhang et al., 2015). However, stable transformation of chloroplast genomes is relatively difficult and limited to a small number of crops compared to nuclear genome transformation, and stable heritability of a transgene in the chloroplast genome through multiple generations is also relatively difficult due to the number of chloroplasts in a cell, the copy number of the genomes in a chloroplast and the tendency of multiple genomes to recombine. Therefore, despite great promise, the efficacy of the reported cross-kingdom RNAi is limited, preventing widespread adoption for effective insect control in crops.

RNAi is highly variable or inefficient in many insects. This is particularly the case for Lepidopteran insect pests (moths and butterflies), as reviewed by Kunte et al. (2019); Terenius et al. (2010); Xu et al. (2016); Zhu and Palli (2020), including in Helicoverpa armigera (Lim et al., 2016). Terenius et al. (2011) noted that reduction in gene expression by RNAi has often proven to be difficult to achieve in Lepidopteran insects, with large variation and requiring high concentrations of dsRNA for success. Gene silencing in epidermal tissues seemed to be the most difficult to achieve. In contrast, gene silencing in Coleopteran insects (beetles) is more often efficient and systemic (Zhu and Palli, 2020). Studies on the mechanism of the dsRNA effect on WCR showed that it was the longer dsRNA molecules of at least 60 basepairs in length, not the siRNA molecules of 20-25 basepairs produced by Dicer processing from the long dsRNA, that were taken up from the plant diet into the insect larval gut cells to induce silencing of the insect target gene (Bolognesi et al., 2012).

### Aim of the Experiment

Based on the experiments described in Example 2 that showed an increased accumulation in plant cells of full-length hpRNA when the molecule has about 20-25% G:U basepairs (hpRNA[G:U]) in its dsRNA region compared to standard hpRNA (hpRNA[Con]), the inventors considered that production of hpRNA[G:U] RNA molecules in plant tissues might therefore provide sufficient dosage of longer dsRNA molecules to be taken up by insects in their diet and thereby decrease target gene activity and possibly provide effective control of the insect pests. To test this concept, the inventors selected, as a model Lepidopteran target gene, one encoding Pheromone Biosynthesis Activating Neuropeptide (PBAN) in the cotton bollworm Helicoverpa armigera (Rafaeli and Jurenka, 2003). Cotton bollworm is a serious pest of cotton through its feeding on developing bolls and other green tissues of the plants. PBAN is a neuropeptide that regulates sex pheromone biosynthesis from the pheromone gland, being produced in the suboesophageal ganglion and acting directly on the PG following adult emergence in a species dependent manner, and therefore required for reproduction. PBAN also has effects earlier in development. The experiment was to test whether hairpin RNA having multiple G:U basepairs in its dsRNA region might be more effective than a corresponding hairpin RNA having only canonical basepairs in the dsRNA region, for expression and accumulation in the plant cells, PBAN target gene suppression in the insect larvae and reduced growth or survival of the larvae, as follows.

### Design of the G:U and Control Hairpin RNAs and Construction of Transgenes Encoding These

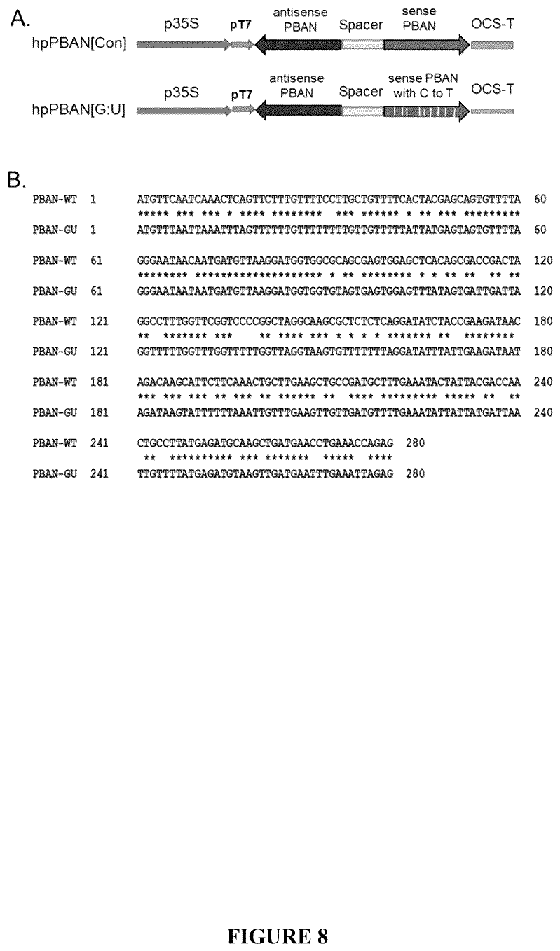

A nucleotide sequence for a cDNA corresponding to a PBAN RNA transcript from H. armigera was identified in a sequence database, namely NCBI Accession No. XM_021343523.1 (gene LOC110382821). The nucleotide sequence included the full protein coding region but not a 3′ UTR sequence. The amino acid sequence of the encoded PBAN polypeptide is provided as SEQ ID NO: 7 and the nucleotide sequence of the corresponding cDNA is set forth as SEQ ID NO: 8 herein. A 280-nucleotide sequence (SEQ ID NO: 9) from the cDNA was selected as the target region, starting at the ATG translation start codon and corresponding to nucleotides 36-315 of SEQ ID NO: 8. For the construct encoding the G:U hairpin RNA, a modified sense sequence was created which had each of the 64 cytidine (C) nucleotides of the sense stand of SEQ ID NO: 9 substituted to thymidines (T), resulting in SEQ ID NO: 10. FIG. 8B shows an alignment of the modified sense sequence with the wild-type sense sequence, showing the cytidine nucleotides that were substituted with thymidines to provide uridines in the RNA molecule, opposite a guanosine in the antisense strand. The modified sense sequence was designed so that 64/280 (22.9%) of the basepairs in the resultant hairpin RNA would be G:U basepairs, spread along the length of the 280 nucleotide sense sequence, where each G:U basepair would have a G in the antisense strand and a U in the sense strand of the dsRNA region. A spacer sequence of 150 nucleotides was also selected, also derived from the PBAN cDNA sequence, to provide for a loop when transcribed to produce the RNA molecule. The spacer sequence corresponded to nucleotides 316-465 of SEQ ID NO: 8 and was placed in sense orientation with respect to the promoter.

Using these sequences, two genetic constructs were designed and made: one encoding the conventional hairpin RNA (designated hpPBAN[Con]) having only canonical basepairing in its dsRNA region and no bulges in the dsRNA region of the molecule. The second construct encoded the modified hairpin RNA designated hpPBAN[G:U] with 22.9% G:U basepairs in the dsRNA region, the only difference with the conventional hairpin RNA. Both genetic constructs thereby contained an inverted repeat of a 280-nucleotide sequence separated by the 150 basepair spacer, with a promoter-antisense sequence-spacer-sense sequence-transcription terminator configuration under the control of a constitutive CaMV 35S promoter. The inverted repeat in the hpPBAN[Con] was therefore a perfect, spaced-apart inverted repeat, whereas the repeat in the hpPBAN[G:U] was imperfect due to the C to T substitutions. Both constructs also contained a T7 promoter sequence immediately upstream of the antisense sequence, to allow for in vitro transcription to produce hairpin RNA as described in Example 1. The constructs did not have an intron in the spacer region. An intron could have been included in that region (Smith et al., 2000) but the inventors chose not to include one. The design of both constructs under the control of a 35S promoter is shown schematically in FIG. 8A.

The genetic sequences were synthesised by a commercial provider and inserted by the inventors separately into the expression vector pART7. The expression cassettes were excised from there and inserted into the T-DNA region of the binary vector pART27 to form the p35S::hpPBAN::ocs-T vectors for plant transformation. This vector has a spectinomycin resistance selectable marker gene for selection in bacteria and a kanamycin resistance gene cassette in the T-DNA for selection of the T-DNA in plant cells (Gleave, 1992). The nucleotide sequences of the DNA coding regions for the hpPBAN[Con] and hpPBAN[G:U] RNA molecules are provided as SEQ ID NOs: 11 and 12, respectively.

In this experiment, the antisense fragments in the two constructs were identical in sequence and fully complementary to the target mRNA along the 280-nucleotide sequence. This meant that any differences in effectiveness could not be due to differences in the antisense sequence, thereby allowing the direct comparison of the two hairpin RNAs. In addition, all antisense sRNAs produced from the hairpins would have full complementarity with the target RNA. The inventors considered that full complementarity to the target RNA was not necessary for G:U hairpins, but in this experiment full complementarity was used.

### Expression of G:U and Control Hairpin RNAs in Plant Cells

The binary vectors were introduced into Agrobacterium tumefaciens strain LBA4404 by electroporation, selecting for spectinomycin resistant colonies. The transformed bacteria were cultured and the resultant cells used to transform Nicotiana tabacum plants of cultivar W38 by standard Agrobacterium-mediated transformation methods as described in Example 1. Kanamycin resistant plantlets were selected in tissue culture for insertion of the T-DNAs and transferred to soil in pots in the glasshouse when they had grown to sufficient size. Twelve independent, regenerated plants (TO plants) were randomly selected for each of the constructs. These plants were analysed by Northern blot hybridisation for expression of the transgenes, demonstrating that they were transformed. Alternately, the plants can be confirmed to be transgenic by PCR amplification of the inserted sequences in DNA isolated from leaf samples.

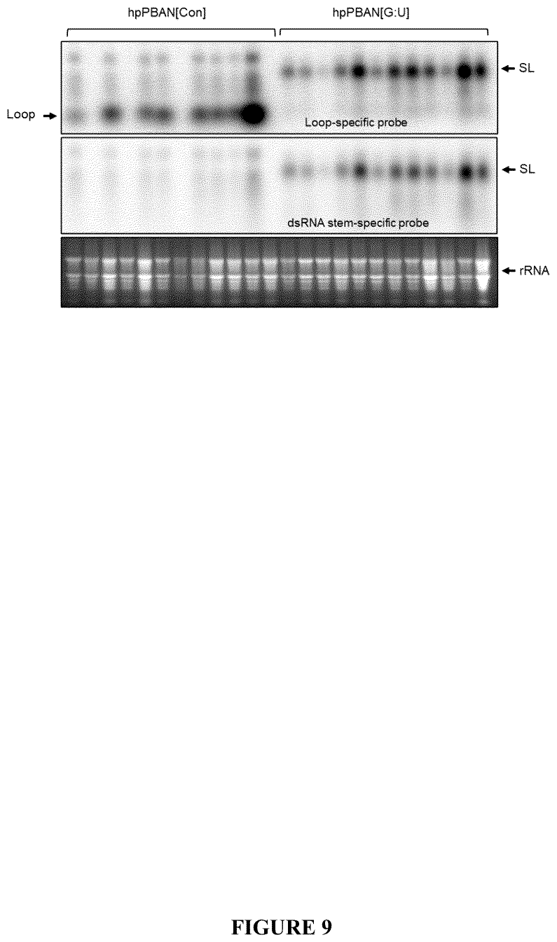

RNA was extracted from leaf samples from each plant as described in Example 1. The total RNA preparations were analysed for hairpin RNA accumulation and processing by Northern blot hybridisation analysis using either a radio-labelled probe which was an RNA complementary to the loop sequence or, after washing the hybridised loop probe off the blot, a probe specific for the dsRNA region (stem) of the hairpin molecules. The results (FIG. 9) showed that the two transgenic populations gave different, distinct RNA accumulation profiles. Eight of the hpPBAN[Con] plants exhibited an intense band which hybridised to the loop probe, but not to the stem probe, and which corresponding in size to processed loop fragments. It was concluded that these hpPBAN[Con] plants accumulating large amounts of the processed loop sequence. Similar observations have been made by the inventors for other conventional hairpin RNAs in transgenic plants (Wang et al., 2008). RNA molecules hybridising to the loop sequence were not detected in the other four hpPBAN[Con] plants indicating either negligible transgene expression, possibly due to transcriptional silencing of the transgene promoter in those plants, or processing or instability of the hpGUS[Con] molecules in those plants involving degradation of the loop sequences.

In contrast, all of the hpPBAN[G:U] plants exhibited abundant amounts of a RNA which hybridised to both the loop and stem probes (FIG. 9 upper and middle panels) and having a length corresponding to the full-length hpPBAN[G:U] transcript or close to that.

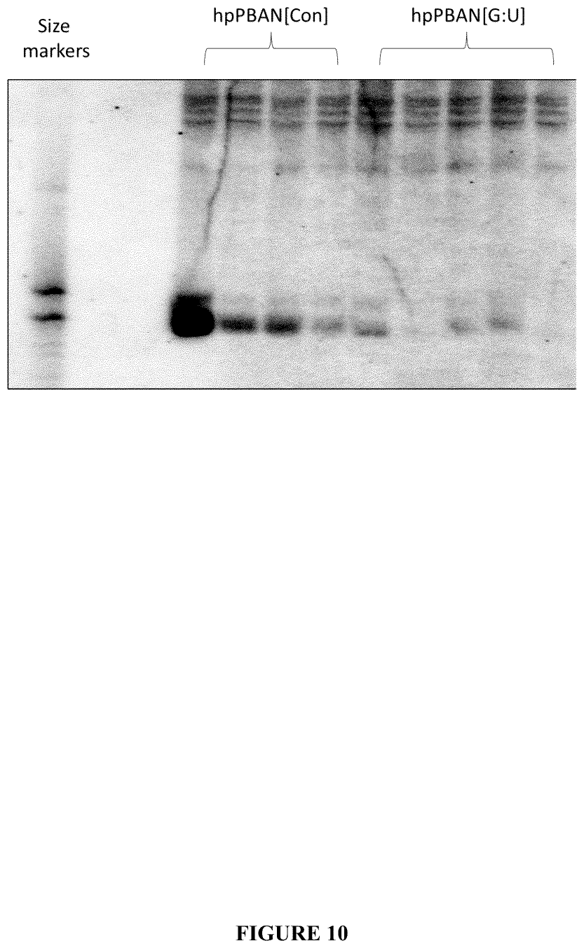

Two further Northern blot hybridisations were performed to detect either the dsRNA region of the hairpin RNA molecules, or to detect small RNA molecules (sRNA) from the dsRNA region of the hairpin RNAs, using a radiolabelled probe from this region. The RNA preparations from four hpPBAN[Con] tobacco plants that expressed the transgene and five hpPBAN[G:U] plants were analysed. The Northern blot hybridisation to detect the longer dsRNA molecules showed that the hpPBAN[G:U] plants accumulated more of the unprocessed dsRNA region containing G:U basepairs than the hpPBAN[Con] plants. The autoradiograph to detect sRNA molecules from the dsRNA region of the molecules is shown in FIG. 10. The RNA preparations from the hpPBAN[Con] plants had significantly greater amounts of 21-mer and 24-mer sRNAs than the hpPBAN[G:U] plants. Small RNAs of these lengths are produced by Dicer processing of dsRNA molecules, in particular by DCL4 and DCL3 polypeptides (Guo et al., 2016).

Essentially the same observations were made when conventional hairpin RNAs and G:U modified hairpin RNAs directed at several other target genes were expressed in transgenic plants, including in A. thaliana and N. benthamiana. It was concluded that the G:U modified hairpin RNAs accumulated in greater amounts in its full-length form, or close to that length, and that it was processed into sRNAs to a lesser extent in the transgenic plants compared to the corresponding, conventional hairpin RNA molecules having only canonical basepairing. The inventors considered that this was due to reduced processing by Dicer enzymes of the hpPBAN[G:U] hairpin RNA in the plant cells. Since the difference between the hairpin RNA molecules was the presence of G:U basepairs in the one molecule, but absent in the other, it was concluded that the different structure of the dsRNA region comprising the G:U basepairs was responsible for the different processing. It was also concluded that the conventional hpRNA transgene was more susceptible to transcriptional silencing in the transgenic plants and that, even when expressed, the conventional hairpin RNA was predominantly processed to form the loop fragment and little full-length hairpin RNA accumulated.

The plants are allowed to self-fertilise and T1 seeds are harvested upon maturity. Multiple T1 seed are sown and the resultant T1 plants are screened for presence of the transgene. Transgenic T1 plants are again grown and allowed to self-fertilise to produce T2 progeny seed and plants. Homozygous T2 progeny plants are selected. This provides transgenic lines which can be maintained over multiple generations without loss of the transgene by segregation. The accumulation of the hairpin RNAs is increased in homozygotes relative to heterozygotes.

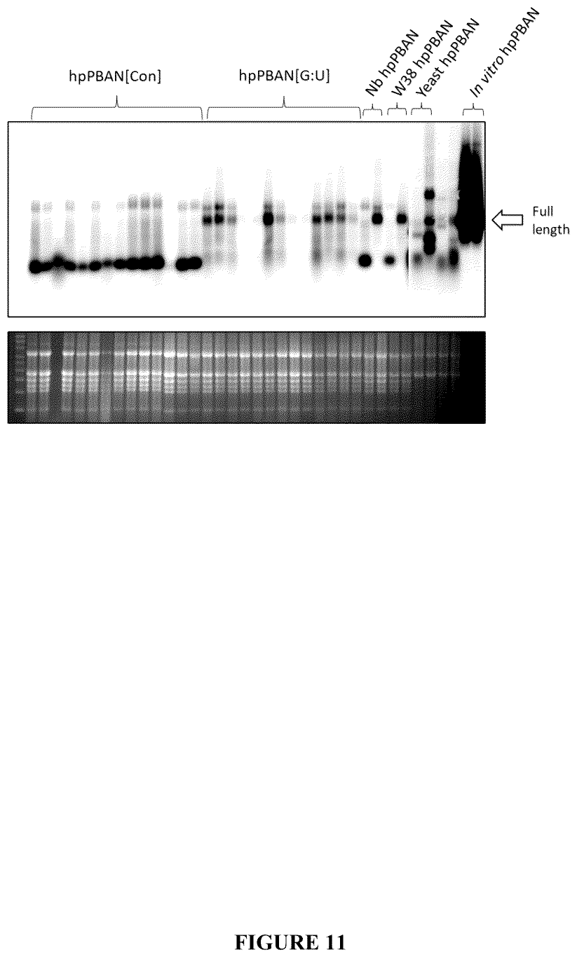

The Agrobacterium strains with the hpPBAN[Con] and the hpPBAN[G:U] construct were also used to separately produce transformed N. benthamiana plants, using the method as described in Example 1, and also used to transform A. thaliana by the floral dip method (Clough and Bent, 1998). Between 13-16 independent, transgenic plants from each transformation procedure were analysed using Northern blot hybridisation with the same loop-specific RNA probe for the PBAN sequence as before. The observed hybridisation patterns for the N. benthamiana and A. thaliana transformants were similar to the corresponding patterns in the N. tabacum plants: the hpPBAN[Con] plants showed a strong hybridising band corresponding to the loop fragment with little or no detectable full-length hairpin RNA, whereas the hpPBAN[G:U] plants accumulated greater amounts of the full-length hpRNA transcript with little of the detached loop fragment (FIG. 11). Again, the construct encoding the G:U modified hairpin RNA clearly yielded increased accumulation of the full-length transcript compared to the construct encoding the hairpin RNA lacking G:U basepairs.

### Expression of G:U and Control Hairpin RNAs in Yeast Cells

To test if full-length G:U modified hairpin RNA could also be produced and accumulated in Saccharomyces cerevisiae cells, the DNA fragments including the hpPBAN[Con] and hpPBAN[G:U] coding sequences were cloned separately into a yeast expression vector pGADT7-AD (Zhong et al., 2019) under the control of an ADH promoter. The resultant expression cassettes were introduced into three different strains of the yeast S. cerevisiae strains HF7C, S288 and SYE6210, and the transformants cultured under standard conditions (Zhong et al., 2019). Northern blot hybridisation analysis of RNAs extracted from the cells using the probe complementary to the loop sequence showed that the hpPBAN[G:U] construct produced much greater amounts of full-length transcript than the hpPBAN[Con] construct in both yeast strains HF7C and SYE6210. The amount of full-length hpPBAN[G:U] in the yeast strain HF7C looked much greater on a weight basis per μg of total cellular RNA than in the stably transformed Nicotiana plant tissues. It was concluded that S. cerevisiae cells could be used to generate large quantities of full-length G:U modified RNAs.

### Example 5: Processing of Insect-Targeted hpRNA[G:U] Molecules after Expression in Plant Cells

### Cellular Locations for Processing of the Hairpin RNAs

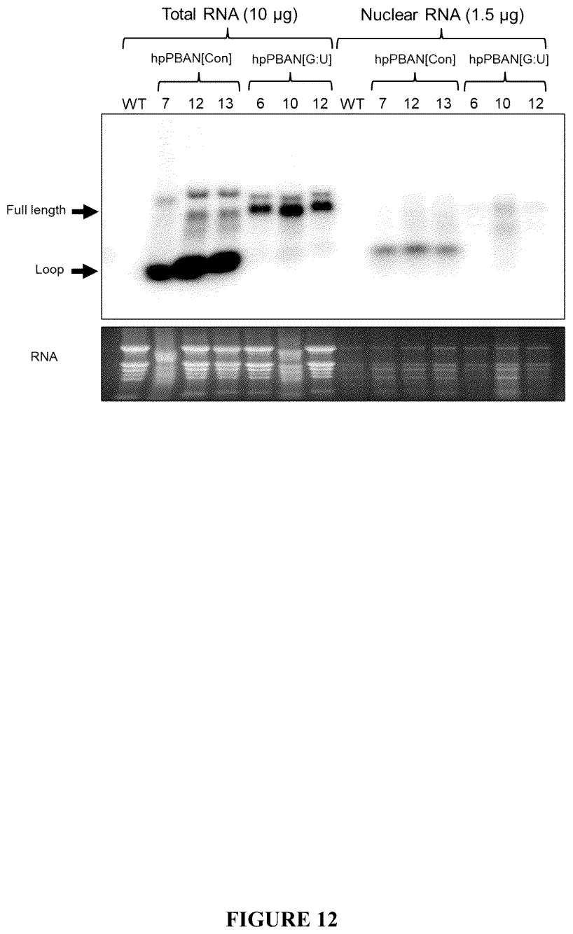

The different profiles of hairpin RNA processing and accumulation between the conventional and G:U modified RNAs in plants led the inventors to investigate if the two types of hairpin RNA were processed and accumulated in different subcellular locations, specifically nuclear or cytoplasmic localisation. Stably transformed N. benthamiana plants expressing either the hpPBAN[Con] or the hpPBAN[G:U] construct were harvested and used for isolation of nuclei with subsequent RNA extraction from the nuclei, as well as for total RNA extraction, as described in Example 1. The RNA preparations from leaf tissue (total RNA) or from leaf nuclei were analysed by Northern blot hybridisation with hybridisation to a radiolabelled probe which was complementary to the loop sequence of the hpRNAs. The blotted membrane was washed and re-probed with a probe that hybridised to a U6 small RNA, a nuclear localised RNA in plant cells, as a loading control. As shown in FIG. 12, the loop fragment from the hpPBAN[Con] transcript was readily detected in the nuclear RNA fraction from the plants, whereas the band corresponding to the full-length hpPBAN[Con] RNA molecules seen in the total RNA fraction were less abundant in the nuclear RNA, relative to the U6 RNA. It was concluded that at least some of the excised loop fragment was present in the nuclei of hpPBAN[Con] plants. In contrast, the dominant full-length hpRNA band seen in the total RNA samples of hpPBAN[G:U] plants was detected at relatively lower amounts in the nuclear RNA fraction, which instead showed two RNA bands that were less than full-length. This result suggested that the hpPBAN[Con] molecules were mostly transcribed and processed in the nuclei to excise the loop region, whereas the hpPBAN[G:U] molecules sustained less loop excision in the nuclei and were relatively more often exported as a full-length RNA to the cytoplasm. Small RNA (sRNA) analyses showed that both hairpin RNAs were processed to produce sufficient 21-24 nucleotide siRNAs for efficient target gene silencing, indicating that both types of hpRNA were processed to at least some extent by a Dicer enzyme (Example 2, and FIG. 10). From this experiment, it was concluded that Dicer processing of the conventional hpRNA occurred more in the nucleus with loop excision, whereas the siRNAs from the G:U modified hpRNA may have come from Dicer processing of full-length transcripts in the cytoplasm and/or from a partial, less efficient processing in the nucleus.

The total RNA preparations are passed through a polyT column to enrich for the polyA-containing RNA molecules. The amount of RNA having a polyA tail produced from the two constructs is compared. The hpPBAN[G:U] construct accumulates more transcripts having a polyA tail than the plants transformed with the hpPBAN[Con] construct.

### Example 6: Effect of hpRNA[G:U] Molecules Targeting a Lepidopteran Insect Gene on the Insect Larvae

### Comparison of the Hairpin RNAs on Insect Development and Survival

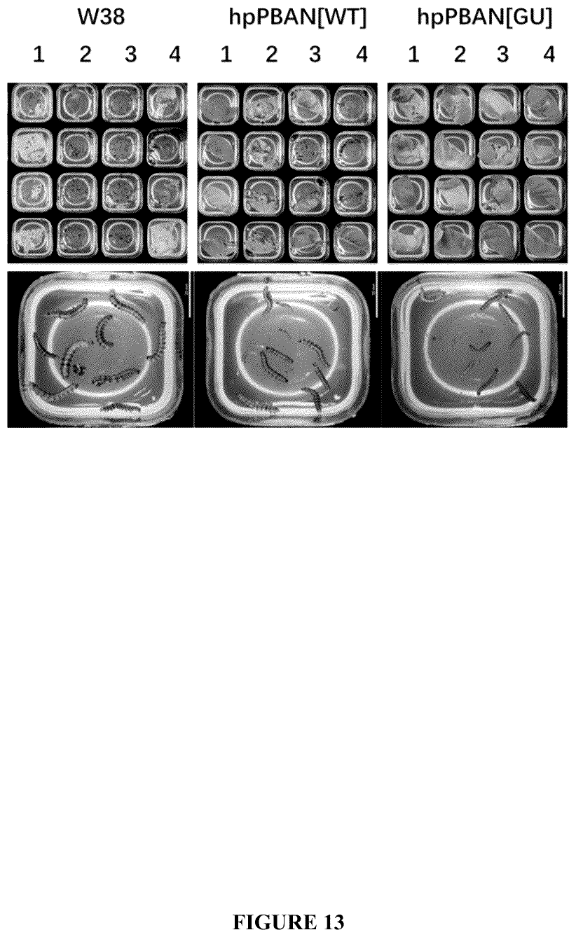

Insect (H. armigera) feeding assays were performed on excised leaves from the transgenic N. tabacum plants produced above, as follows: H. armigera eggs were placed on a filter paper to hatch at 26° C. Four independently transformed tobacco TO plants were chosen for the feeding assay, with two leaves being excised from each plant and each leaf cut into two pieces, making four replicates for each individual plant. Using a soft paintbrush, a single, newly hatched larva, not more than 10 hr after hatching, was transferred to the leaf piece placed inside an 0.8% agar plate. The plate was sealed with porous plastic membrane and kept at 26° C. for 7 days. At this time, the extent of leaf damage in each sample was scored and the larval weights were measured. As shown in FIG. 13, the hpPBAN[G:U] leaf pieces yielded the least damage, with the lowest average larval length and weight, followed next by the hpPBAN[Con] leaf pieces, and lastly the wild-type, untransformed W38 leaf pieces which were completely eaten. This result indicated that the hpPBAN[G:U] plants were more resistant to H. armigera, in terms of reduced larval growth, than the hpPBAN[Con] plants.

### Example 7: Development of a Transient Expression System for hpRNA[G:U] Molecules

The production of transgenic plants expressing hairpin RNAs designed as described in Examples 4 and 5 is a long and laborious process for many plant species, for example transformation of cotton plants. The inventors therefore sought a more rapid system for expressing hairpin RNAs in plant cells to enable higher throughput testing, in particular to test a variety of candidate target genes. In an attempt to develop such a system, the processing and accumulation of hpPBAN[G:U] and hpPBAN[Con] hairpin RNAs were compared using a transient expression system in N. benthamiana leaves, using Agrobacterium-mediated infiltration of the genetic constructs encoding the hairpin RNAs. In this system, multiple copies of the T-DNAs carrying the transgene(s) of interest are introduced into the leaf cells and most copies do not integrate into the nuclear genome, but nevertheless are expressed in the nucleus. The inventors considered that such an in planta expression system could provide a rapid system for testing the efficacy of candidate hpRNA constructs for insect pest control, where the insects could feed on the infiltrated leaf tissues expressing and accumulating the hpRNAs. This could be done over 4-5 days post infiltration, so providing a rapid test system.

These experiments were conducted as described below with the use of co-expression of a Cucumber Mosaic Virus (CMV) 2b silencing suppressor (Goto et al., 2007; Shen et al., 2015) to increase and extend expression levels of the RNAs from the hairpin-encoding genes. This silencing suppressor was selected for these experiments as it has been reported to suppress both local and systemic silencing by binding to siRNA molecules produced by Dicer, while binding only weakly to miRNA molecules (Guo and Ding, 2002; Goto et al., 2007). This is in contrast to the more commonly used viral silencing suppressor p19 from tomato bushy stunt virus (Voinnet et al., 2003) which binds more strongly to both siRNA and miRNA molecules. The CMV 2b silencing suppressor has also been reported to bind to and inhibit the RNA cleavage activity of Argonaute 1 (AGO1) protein (Zhang et al., 2006; Fang et al., 2016) that is required for multiple pathways of RNAi silencing, which may be its primary mode of action. The 2b protein expressed from the CMV genome has been reported to drastically reduce the accumulation of 21-, 22-, and 24-nucleotide classes of viral siRNAs produced by DCL4, DCL2 and DCL3, respectively (Diaz-Pendon et al., 2007).

The inventors therefore considered that co-infiltration with a construct encoding the CMV 2b suppressor of silencing would inhibit the co-suppression that can occur after Agrobacterium-mediated gene introduction, thereby enhancing and prolonging the expression of the introduced transgenes such as the hpRNA transgenes, without interfering with the Dicer-mediated processing of the hairpin RNAs. The nucleotide sequence of the protein coding region for the CMV 2b polypeptide and the amino acid sequence of the polypeptide are provided as SEQ ID NOs: 13 and 14, respectively. The DNA sequence was linked to a 35S promoter to provide high level, constitutive expression of the silencing suppressor in the plant tissues.

To test the expression system, the hpPBAN[Con] and hpPBAN[G:U] constructs were infiltrated separately using A. tumefaciens into N. benthamiana leaves, each in the presence of a separate construct encoding the CMV 2b RNA silencing suppressor, also introduced via A. tumefaciens. The accumulation and processing of each hairpin RNA was analysed 3 days post infiltration by Northern blot hybridisation using a probe that was complementary to the loop sequence. As for the stably transformed plants described above, the RNA from the hpPBAN[Con] construct exhibited mostly the detached loop fragment, whereas the longer, full-length transcripts were not detected. In contrast, the hpPBAN[G:U] construct produced and accumulated much greater levels of the full-length hairpin RNA. However, unlike in the stably transformed plants, a shorter fragment was also observed to accumulate which was larger than the loop fragment of hpPBAN[Con] but shorter than the full length transcript. The size of the additional fragment, probably containing some sequence from the dsRNA region of the molecule, indicated that the loop of the G:U hairpin was processed differently in the transient expression system than the loop of the conventional, canonically basepaired hairpin RNA. Importantly however, the infiltrated tissues that received and expressed the transgene encoding the G:U modified hairpin RNA accumulated much higher levels of hpRNA-derived RNA, including the full-length transcript, than the same transgene in the stably transformed plants. It was concluded that the transient expression system in the N. benthamiana plants provided a useful system for preparing large amounts of G:U modified hairpin RNA and for assaying the potential efficacy of G:U hairpin RNA molecules for effects on insects that could ingest the leaf tissues.

### Example 8: Candidate Target Genes in a Lepidopteran Insect for Targeting with hpRNA[G:U] Molecules

### Introduction

A variety of target genes in H. armigera have been tested for suppression with dsRNA by oral delivery to larvae, with highly variable results that are often transient and require large quantities of dsRNA to be delivered (Lim et al., 2016). The factors that contribute to the variability include larval gut conditions which quickly degrade dsRNA in the digestive system, in particular the alkaline pH of the midgut contents and presence of RNases. Other factors include an apparent lack of a substantial RNAi amplification mechanism in H. armigera, evidenced by an absence of a gene for RNA-dependent RNA polymerase (RdRp) in the insect genome, target genes not being expressed in midgut cells, insufficient expression of SID-1 and SID-2 genes which are responsible for dsRNA uptake and transport, and potential blocks to the processing and release of RNA from vesicles after being taken up by endocytosis. Lim et al. (2016) recommended targeting genes which are expressed in the midgut with lower transcript abundance.

Based on the results of the experiments described above, the inventors selected a range of nine candidate target genes in addition to PBAN (Example 4) for testing with G:U modified hairpin RNA and comparison with conventional hairpin RNA. This included three genes that have not been previously reported as targets for RNAi, according to the inventors' knowledge. The ten genes including PBAN are listed in Table 1.

TABLE 1
Candidate target genes in Helicoverpa armigera tested
for down-regulation with modified RNA molecules.

| | | | SEQ ID | Target |
|---|---|---|---|---|
| Target | | Expression | No of | region |
| ID | Gene name | pattern | CDNA | length (nt) |
| PBAN | Pheromone biosynthesis | Head, ventral | 8 | 200 |
| | activating neuropeptide | nerve | | |
| AChE1 | Acetylcholinesterase | Body wall, | 16 | 494 |
| | | glial cells | | |
| Ha1 | Ecdysone receptor | Whole body | 22 | 300 |
| Ha2 | Coatomer beta | Midgut | 28 | 300 |
| | | epithelial cells | | |
| Ha3 | Molt-regulating | Whole body | 34 | 300 |
| | transcription factor | | | |
| | (HR3) | | | |
| Ha4 | v-ATPase | Midgut | 40 | 300 |
| Ha5 | Trypsin-like serine | Midgut | 46 | 300 |
| | protease | | | |
| Ha6 | Synaptic vesicle | Foregut, | 52 | 300 |
| | glycoprotein | hindgut, adult | | |
| Ha7 | Troponin C | Foregut, adult | 58 | 300 |
| Ha8 | Titin | Foregut, | 64 | 300 |
| | | hindgut, adult | | |

For each of the genes Ha1-Ha3, Ha5, Ha6 and Ha8, a region of the mRNA was selected which had about 25% C and about 25% A nucleotides in its sequence, which the inventors considered to be preferred. For Ha4 and Ha7, the selected region had about 25% C and about 19% A or about 28% A nucleotides, respectively. Each selected region was 300 nucleotides in length, but any length of at least 50 nucleotides was considered to be workable. It was considered that, for specific target gene down-regulation, the region to be selected should not have any homologous sequence in common with other genes that should not be down-regulated, where a homologous sequence is defined in this context as having at least 19 identical nucleotides in a contiguous 21-nucleotide stretch in the target region. Alternatively, when it is desired to down-regulate related target genes, such as members of a gene family, a target sequence that contains one or more homologous regions is selected.

### Acetylcholinesterase (AChE)

Acetylcholinesterase is an enzyme expressed in the central nervous system of insects. The enzyme functions by hydrolysing acetylcholine into choline and acetic acid (Oakeshott et al., 2005) and thereby has an important role in termination of the signal transmission process in the nervous system. Besides neurotransmission, AChE is thought to have other roles in cellular processes such as modulation of cellular interactions, apoptosis, cell adhesion and the genesis of synaptic connections (Soreq and Seidman, 2001; Zhang and Shi, 2002). The expression of AChE has been reported in the insect body wall, anterior body structures and appendages during embryonic development, and strong expression occurs in longitudinal glia and glial cells during larval development as well as nerve tissues (Bicker et al., 2004). In many insects including H. armigera, two homologous AChE genes are present, namely AChE1 and AChE2 (Lee et al., 2006). AChE1 transcripts are much more abundant than AChE2, and therefore the AChE1 gene was selected by the present inventors for targeting by the modified hairpin RNA molecules. Kumar et al. (2009) silenced the AChE1 gene by feeding H. armigera larvae an artificial diet supplemented with chemically synthesized, sense and antisense hybrid RNA-DNA molecules of 19 basepairs in length that had been annealed, corresponding to nucleotides 1657-1675 of SEQ ID NO: 16 herein. High concentrations of the molecules at 25-50 nM resulted in slowed development and a reduction in survival of the larvae from about 85% to 60%.

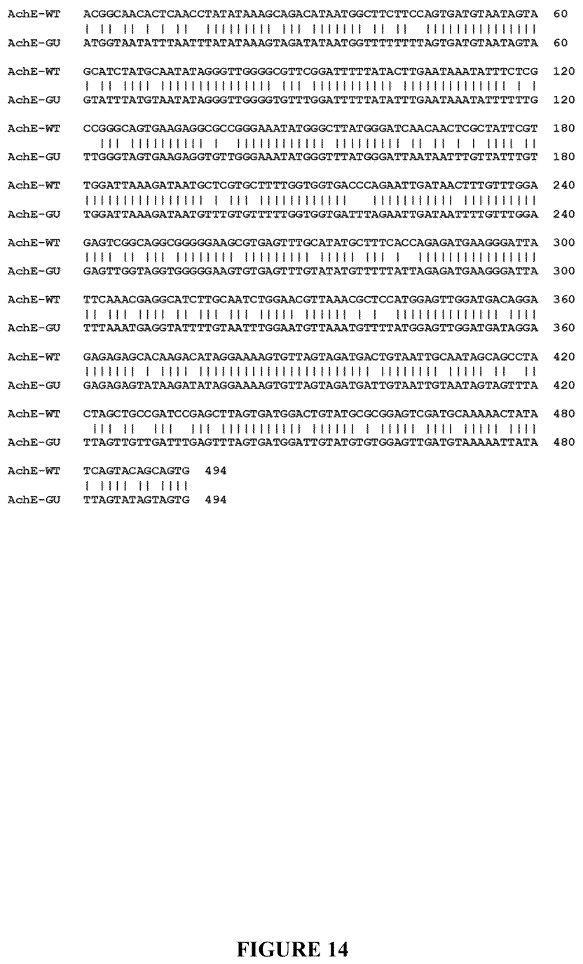

A nucleotide sequence encoding an AChE1 polypeptide (SEQ ID NO: 15) was obtained from Ren et al. (2002), Genbank Accession No. AF369793, provided herein as SEQ ID NO: 16. A target sense sequence of 494 nucleotides was selected (SEQ ID NO: 17), corresponding to nucleotides 895-1388 of SEQ ID NO: 16. This target region was selected randomly, without considering the percentage of C nucleotides in the sequence. A modified sense sequence was designed and made by substituting 86 cytidine nucleotides (17.4%) in SEQ ID NO: 17 for thymidine nucleotides, resulting in SEQ ID NO: 18. An alignment of SEQ ID NO: 17 and SEQ ID NO: 18 showed the position of the substitutions, spread across the 494-nucleotide sense sequence (FIG. 14).

A genetic construct encoding the G:U modified hairpin RNA designated hpAChE[G:U] was designed and synthesised. A control genetic construct was also made, encoding a conventional, canonically basepaired hairpin RNA using the wild-type sense sequence i.e. without the C to T substitutions, but otherwise identical to hpAChE[G:U]. The control construct was designated hpAChE[Con]. The order of components in both genetic expression constructs was: 35S promoter-antisense sequence-spacer-sense sequence-transcription terminator/polyadenylation region. The spacer sequence of 262 nucleotides, complementary to nucleotides 633-894 of SEQ ID NO: 16, encoded the loop in both hairpin RNA molecules. The nucleotide sequences for the DNA molecules encoding the hairpin RNAs are provided as SEQ ID NO: 19 (hpAChE[Con]) and SEQ ID NO: 20 (hpAChE[G:U]). Two other constructs were also made with the same sense and antisense sequences, one encoding an ledRNA with canonical basepairing (ledAChE[Con]) and the other the same except comprising the G:U basepairs (ledAChE[G:U]).

### Ecdysone Receptor

Another target gene selected by the inventors was one encoding Ecdysone Receptor (EcR), also designated herein as Ha1. The EcR protein is a master regulator of insect development. It is involved in the regulation of all developmental stages of the insect life cycle, in particular embryogenesis, hatching, insect growth, molting and metamorphosis. The EcR protein forms a heterodimer with another protein, ultraspiracle (Usp), to form a nuclear receptor for the steroid hormone α-ecdysone and its hydroxylated derivative, 20-hydroxyecdysone, referred to herein collectively as ecdysone. The receptor is required for the steroid hormone signalling pathway that controls insect development (Thummel, 1995; Riddiford et al., 2000). When bound to ecdysone, the EcR-Usp complex functions as an active transcription factor to regulate several genes at the top of a hierarchy of genes as well as multiple, more downstream genes. The receptor complex does this by binding to DNA elements upstream of the target genes at different developmental stages (Uyehara and Mckay, 2019). The early genes that are regulated by EcR include those encoding the Broad-Complex, E74A and E75B proteins, each of which in turn also regulate downstream genes. The early genes activate a group of late genes which regulate metamorphosis during insect development including the processes of cell proliferation and differentiation, cell death and cuticle formation. The EcR gene is also induced directly by ecdysone, providing an autoregulatory loop that increases the level of receptor protein in response to its ligand.

In Drosophila, a single gene encodes three isoforms of EcR, namely EcR-A, -B1 and -B2, through the use of two promoters and alternative RNA splicing. These differ in their N-terminal domains and have different temporal and spatial expression patterns, indicating distinct roles in development (Talbot et al., 1993; Li and Bender, 2000; Hu et al., 2003). Mutations in the EcR locus that inactivate all three isoforms are embryonically lethal (Bender et al., 1997). Both dsRNA- and artificial miRNA-mediated silencing of the gene encoding EcR in H. armigera have resulted in increased larval mortality and molting defects. Zhu et al. (2012) produced a dsRNA using a construct having two T7 promoters in E. coli strain HT115, a strain which is deficient in bacterial RNAse III. The dsRNA was complementary to nucleotides 245-726 of GenBank Accession No. EU526831, corresponding to nucleotides 453-934 of SEQ ID NO: 22 herein. When fed to H. armigera larvae via an artificial diet, the dsRNA caused increased mortality and developmental defects. Further, a genetic construct for producing a conventional hairpin RNA targeting the same region was introduced into transgenic tobacco plants, for expressing a HaEcR hpRNA. These plants were resistant to H. armigera feeding relative to untransformed plants, with less plant damage. The larvae also showed molting defects and reduced body size. In another study targeting EcR, Yogindran and Rajam (2016) fed 2nd instar larvae of H. armigera with E. coli expressing a genetic construct to produce an amiRNA of 21 nucleotides that was complementary to the EcR transcript. Specifically, the amiRNA molecule was complementary to nucleotides 264-284 of SEQ ID NO: 22 herein. In the amiRNA precursor molecule, the 21-nucleotide stretch was basepaired to a sense sequence by 16 non-contiguous basepairs, interrupted with 5 non-basepaired positions in the dsRNA region. Yogindran and Rajam (2016) reported about 20% to 50% mortality of the larvae when fed the bacteria, with reduced pupal weight and egg laying. Using a similar amiRNA targeting the same region of the EcR mRNA but expressed in transgenic tomato plants, Yogindran and Rajam (2021) reported that the transgenic tomato leaves, when fed to H. armigera larvae, caused a reduction in EcR target gene transcripts and affected the overall growth and survival of the larvae. The precursor miRNA for that amiRNA had 18 non-contiguous basepairs with two non-basepaired positions.

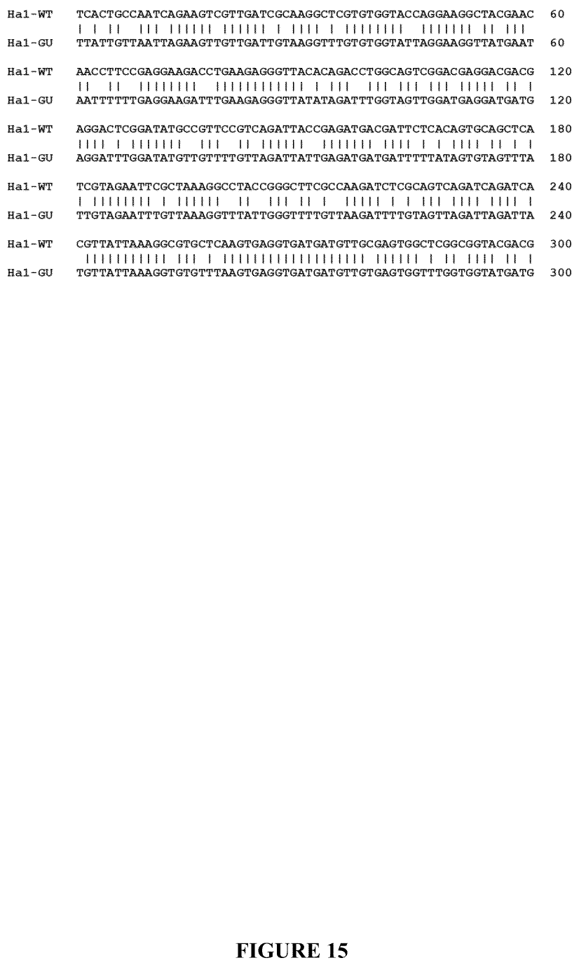

A nucleotide sequence for a cDNA corresponding to the mRNA encoding H. armigera ecdysone receptor (EcR, designated herein as Ha1) was obtained from Genbank Accession No. KY328717.1, provided herein as SEQ ID NO: 22, encoding a polypeptide with the amino acid sequence SEQ ID NO: 21. A target sense sequence of 300 nucleotides was selected (SEQ ID NO: 23), containing 24.3% C and 25.7% A, corresponding to nucleotides 1240-1539 of SEQ ID NO: 22. A modified sense sequence was designed and made by substituting 73 cytidine nucleotides (to provide 24.3% G:U basepairs) in SEQ ID NO: 23 for thymidine nucleotides, resulting in SEQ ID NO: 24. An alignment of SEQ ID NO: 23 and SEQ ID NO: 24 shows the position of the substitutions, spread across the 300 nucleotide sense sequence (FIG. 15).

A genetic construct encoding the G:U modified hairpin RNA designated hpHa1[G:U] was designed and synthesized with the order of components 35S promoter-antisense sequence-spacer-sense sequence-transcription terminator/polyadenylation region. A genetic construct encoding the control, conventional hairpin RNA designated hpHa1[Con] was designed and synthesised with the same order of components except with the unmodified sense sequence instead of the modified sense sequence. The spacer sequence of 150 nucleotides, complementary to nucleotides 1540-1689 of SEQ ID NO: 22, encoded the loop in both RNA molecules. The nucleotide sequences for the DNA molecules encoding the transcripts are provided as SEQ ID NO: 25 (hpHa1[Con]) and SEQ ID NO: 26 (hpHa1[G:U]), not including 5′ leader and 3′ trailer/polyadenylation sequences.

### Coatomer Beta Subunit

Another target gene, designated herein as Ha2, encodes a coatomer beta subunit, which is a protein that functions in the Golgi apparatus of insect cells. The Golgi complex functions to modify, transport, sort and package proteins and lipids into vesicles which are delivered to various cellular compartments such as the plasma membrane, the extracellular medium and the endosomal/lysosomal compartments, particularly between the Golgi complex and the endoplasmic reticulum (Kondylis and Rabouille, 2009). Normal functioning of the Golgi complex involves the coating of vesicles with the Coatomer protein complex (COPI). Other than its role in protein transport, COPI is also involved in cell division and lipid homeostasis (Beck et al., 2009; Beller et al., 2008). COPI includes seven subunits (α, β, β′, γ, δ, ε, ξ), each of which, when absent in insects, reduce fitness and increase mortality rates. In Drosophila, loss-of-function mutants for COPI subunits have several secretory defects which are lethal in embryos (Jayaram et al., 2008). β-COPI is a subunit of COPI and is essential for the transport of proteins from the endoplasmic reticulum to the Golgi complex (Pepperkok et al., 1993). Down-regulation of the gene encoding β-COPI in the spider mite Tetranychus urticae (Trombidiformes) through artificial feeding of in vitro synthesised dsRNA of 513 bp in length resulted in 65% mortality after 5 days (Kwon et al., 2013). Colorado potato beetles (Leptinotarsa decemlineata, Coleoptera) fed with dsRNA targeting the gene encoding β-COPI showed reduction in its mRNA and an increase in mortality (Zhu et al., 2011). That dsRNA was 228 bp in length and had been synthesised in E. coli or by in vitro synthesis. Mao et al. (2015) reduced expression of the coatomer β and v-ATPase A genes in H. armigera using siRNA feeding-based assays, using a mixture of siRNAs having 21 canonical basepairs supplied at 10 μg/cm². However, 5 g/cm² of siRNA failed to cause significant lethality in treated larvae. These results demonstrate that targeting of β-COP1 through RNAi can disrupt the integrity of the Golgi complex and disturb protein secretion impacting insect fitness.

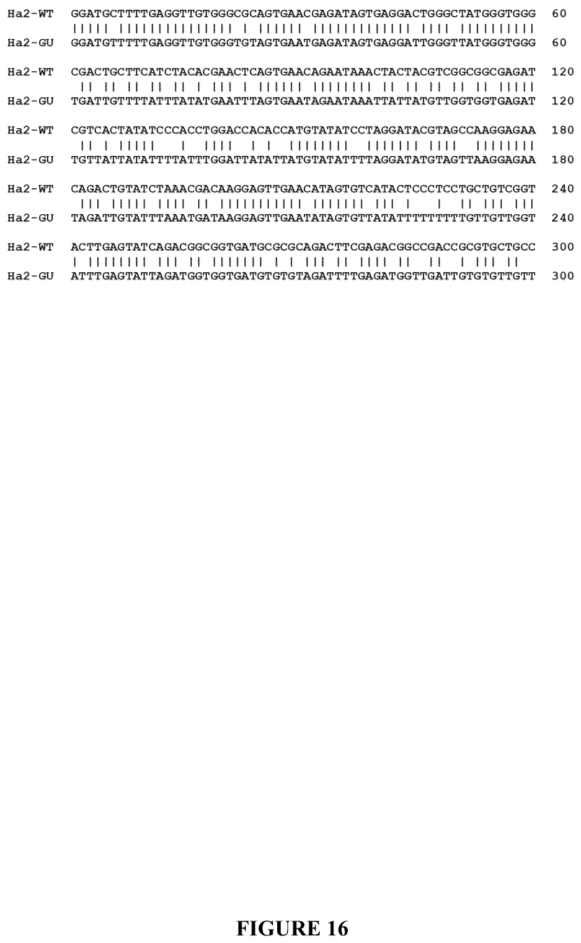

A nucleotide sequence for a cDNA corresponding to the mRNA encoding H. armigera coatomer beta subunit (designated herein as Ha2) was obtained from Genbank Accession No. XM_021339008.1, provided herein as SEQ ID NO: 28, encoding a polypeptide with the amino acid sequence SEQ ID NO: 27. A target sense sequence of 300 nucleotides was selected (SEQ ID NO: 29), containing 24.3% C and 25.7% A, corresponding to nucleotides 1632-1931 of SEQ ID NO: 28. A modified sense sequence was designed and made by substituting 73 cytidine nucleotides (24.3%) in SEQ ID NO: 29 for thymidine nucleotides, resulting in SEQ ID NO: 30. An alignment of SEQ ID NO: 29 and SEQ ID NO: 30 shows the position of the substitutions, spread across the 300 nucleotide sense sequence (FIG. 16).

A genetic construct encoding the G:U modified hairpin RNA designated hpHa2[G:U] was designed and synthesised with the order of components 35S promoter-antisense sequence-spacer-sense sequence-transcription terminator/polyadenylation region. A genetic construct encoding the control, conventional hairpin RNA designated hpHa2[Con] was designed with the same order of components except with the unmodified sense sequence. The spacer sequence of 150 nucleotides, complementary to nucleotides 1932-2081 of SEQ ID NO: 28, encoded the loop in both RNA molecules. The nucleotide sequences for the DNA molecules encoding the transcripts are provided as SEQ ID NO: 31 (hpHa2[Con]) and SEQ ID NO: 32 (hpHa2[G:U]), not including 5′ leader and 3′ trailer/polyadenylation sequences.

### Molt-Regulating Transcription Factor (HR3)

As described above, the steroid hormone ecdysone controls multiple developmental transitions in insects including the larval to adult metamorphosis. The Ecdysone induced moult-regulating transcription factor HR3, designated herein as Ha3, is a member of the nuclear receptor superfamily which when expressed triggers a cascade of gene expression changes required for moulting (Palli et al., 1992). In Drosophila and Manduca sexta, HR3 expression is induced by ecdysone at various stages in development including the transition from larvae to prepupae (Koelle et al., 1992; Parvy et al., 2014). HR3 inhibits early response factors while at the same time inducing the expression of βFtz-F1, a key factor in the pre-pupal to pupal transition (Parvy et al., 2014) and other factors. In Drosophila, HR3 is also required for normal embryonic development of the central nervous system and for hatching (Carney et al., 1997). HR3 is particularly important for the formation of tissues such as wings and the epidermis. Null HR3 mutants are embryonic lethal, associated with defects in the development of the nervous system and muscle (Carney et al., 1997). In H. armigera, the HR3 polypeptide of 556 amino acids is widely expressed in a variety of tissues including the midgut, epidermis and fat bodies (Zhao et al., 2004). In Locusta migratoria nymphs, injecting a dsRNA targeting HR3 inhibited molting and led to death of the insect (Zhao et al., 2018). Expression of the HaHR3 gene was reduced in H. armigera by feeding any one of four dsRNAs produced in E. coli, or feeding transgenic tobacco expressing a hairpin RNA, resulting in developmental deformity and larval lethality (Xiong et al., 2013). The hairpin RNA targeted the region of nucleotides 1-450 of SEQ ID NO: 34 herein. In a different study with six different target genes, Jaiwal et al. (2020) fed second instar H. armigera larvae on an artificial diet containing dsRNA produced by in vitro transcription against the HaHR3 gene, resulting in various deformities and larval mortality. The dsRNA targeted the region of nucleotides 934-1440 of SEQ ID NO: 34 herein.

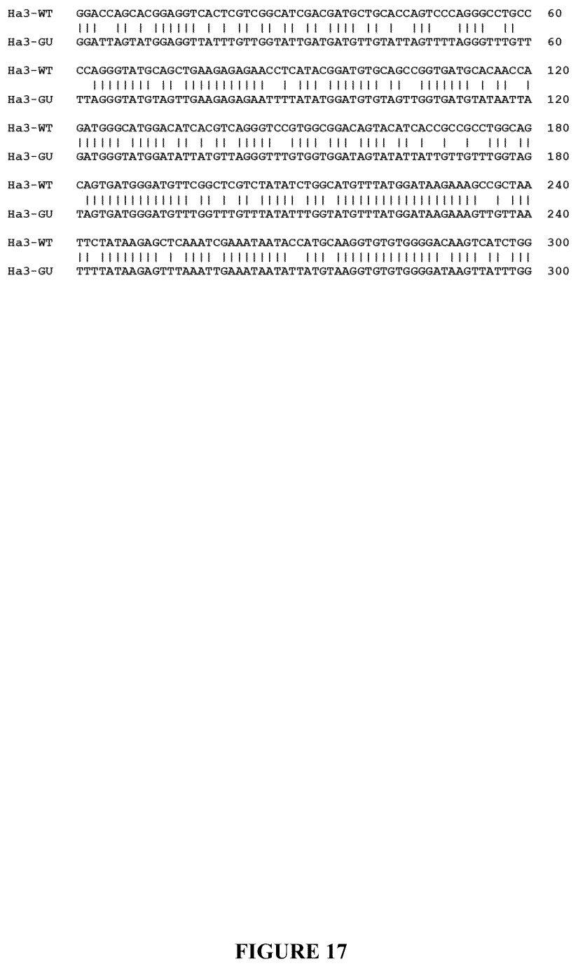

A nucleotide sequence for a cDNA corresponding to the mRNA encoding H. armigera molt-regulating transcription factor (HR3) (designated herein as Ha3) was obtained from Genbank Accession No. FJ009448, provided herein as SEQ ID NO: 34, encoding a polypeptide with the amino acid sequence SEQ ID NO: 33. A target sense sequence of 300 nucleotides was selected (SEQ ID NO: 35), corresponding to nucleotides 48-347 of SEQ ID NO: 34. A modified sense sequence was designed and made by substituting 75 cytidine nucleotides (25%) in SEQ ID NO: 35 for thymidine nucleotides, resulting in SEQ ID NO: 36. An alignment of SEQ ID NO: 35 and SEQ ID NO: 36 shows the position of the substitutions, spread across the 300 nucleotide sense sequence (FIG. 17).

A genetic construct encoding the G:U modified hairpin RNA designated hpHa3[G:U] was designed and synthesised with the order of components 35S promoter-antisense sequence-spacer-sense sequence-transcription terminator/polyadenylation region. A genetic construct encoding the control, conventional hairpin RNA designated hpHa3[Con] was designed with the same order of components except with the unmodified sense sequence. The spacer sequence of 150 nucleotides, complementary to nucleotides 348-497 of SEQ ID NO: 34, encoded the loop in both RNA molecules. The nucleotide sequences for the DNA molecules encoding the transcripts are provided as SEQ ID NO: 37 (hpHa3[Con]) and SEQ ID NO: 38 (hpHa3[G:U]), not including 5′ leader and 3′ trailer/polyadenylation sequences.

### V-ATPase

The vacuolar-type H⁺-ATPase, commonly referred to as the v-ATPase, catalyses the hydrolysis of ATP to ADP and phosphate and uses the energy associated with this reaction to drive ions across cell membranes, thereby regulating pH in multiple organelles (Wieczorek et al., 1989; Wieczorek et al., 2009). The complex drives H⁺ from inside the cell to the lumen and thereby enables cells to import ions such as K⁺ and other molecules, including osmotically obliged water. V-ATPase is a complex of multiple subunits including the VO and V1 sub-complexes that is highly conserved in insects and is found in nearly all epithelial tissues including the gut, salivary glands, testes, ovarioles, testes and Malpighian tubules (Wieczorek et al., 2003; Zeng et al., 2021). In Lepidopteran insects, the V1 complex of the plasma membrane v-ATPase contains eight different subunits designated A to H, whereas the VO complex consists of the four different subunits a, c, d and e (Merzendorfer et al., 2000). V-ATPase is also associated with alkalization of the midgut in Lepidoptera to pH values as high as 11 or 12 (Azuma et al., 1995). Early work in Drosophila demonstrated lethality to larvae when v-ATPase was mutated or disrupted (Davies et al., 1996; Allan et al., 2005). South American tomato pinworm (Tuta absoluta, Lepidoptera) larvae fed on leaves exogenously applied with 50 μg/cm² of dsRNA, produced by in vitro transcription, targeting the v-ATPase B gene had significantly increased mortality rate after 3 days feeding (Ramkumar et al., 2021). Mao et al. (2015) targeted the mRNA encoding v-ATPase A in H. armigera larvae with four different siRNAs each having 19 basepairs, exogenously applied on the surface of tobacco leaves and fed to the larvae. This resulted in a reduction in size and an increase in mortality after 10 days treatment. The siRNAs were synthesised as sense and antisense RNAs and hybridised, and targeted the regions corresponding to nucleotides 553-570, 115-1171, 1288-1306 and 1579-1597 of SEQ ID NO: 40 herein. In another study, Jin et al. (2015) transformed chloroplasts of tobacco plants with a genetic construct to express a short hairpin RNA targeting the v-ATPase A gene of H. armigera and observed reduced larval growth and pupation rates. The short hairpin had 19 canonical basepairs and targeted the region of the mRNA corresponding to nucleotides 711-729 of SEQ ID NO: 40 herein.

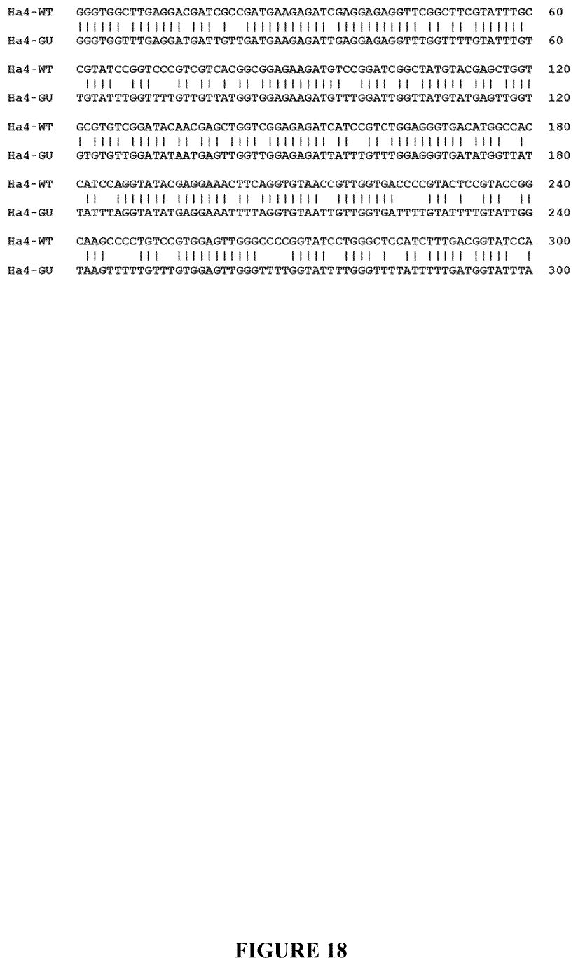

A nucleotide sequence for a cDNA corresponding to the mRNA encoding H. armigera v-ATPase A (designated herein as Ha4) was obtained from Genbank Accession No. XM_021325374.1 (LOC110369820), transcript variant X1, provided herein as SEQ ID NO: 40, encoding a polypeptide with the amino acid sequence SEQ ID NO: 39. A target sense sequence of 300 nucleotides was selected (SEQ ID NO: 41), corresponding to nucleotides 164-463 of SEQ ID NO: 40. This sequence has 25.7% C and 19.3% A nucleotides. A modified sense sequence was designed and made by substituting 77 cytidine nucleotides (25.7%) in SEQ ID NO: 41 for thymidine nucleotides, resulting in SEQ ID NO: 42. An alignment of SEQ ID NO: 41 and SEQ ID NO: 42 shows the position of the substitutions, spread across the 300-nucleotide sense sequence (FIG. 18).

A genetic construct encoding the G:U modified hairpin RNA designated hpHa4[G:U] was designed and synthesised with the order of components 35S promoter-antisense sequence-spacer-sense sequence-transcription terminator/polyadenylation region. A genetic construct encoding the control, conventional hairpin RNA designated hpHa4[Con] was designed with the same order of components except with the unmodified sense sequence. The spacer sequence of 150 nucleotides, complementary to nucleotides 464-613 of SEQ ID NO: 40 encoded the loop in both RNA molecules. The nucleotide sequences for the DNA molecules encoding the transcripts are provided as SEQ ID NO: 43 (hpHa4[Con]) and SEQ ID NO: 44 (hpHa4[G:U]), not including 5′ leader and 3′ trailer/polyadenylation sequences.

### Trypsin-Like Serine Protease

Trypsin-like serine proteases are major digestive enzymes found in the gut of insect larvae (Sui et al., 2009; Zhu et al., 2003). Reduction in tryspin-like proteases results in poor protein digestion and a lack of amino acids for growth. The expression of the gene encoding the protease in H. armigera is tissue-specific: the mRNA is expressed in the midgut and not in the head-thorax, integument, fat body and haemocytes from 5th instar larvae (Sui et al., 2009). In H. armigera, the protease is negatively regulated through a miRNA, namely har-miR-2002b. The miRNA is expressed at high levels up until the 4th instar after which the miRNA expression level decreases and the tryspin-like protease expression increases (Jayachandran et al., 2013). Over-expressing har-miR-2002b by supplying a mimic miRNA to the food of H. armigera larvae resulted in a number of developmental changes within the insect. Insects fed the mimic miRNA displayed a reduction in weight, a delay in entering the pupal stage and reduced fecundity compared to control insects (Jayachandran et al., 2013). The present inventors realised from the observation that H. armigera development is sensitive to fluctuations in expression level of the trypsin-like protease that this gene is a preferred target for testing the hpRNA[G:U] molecule.

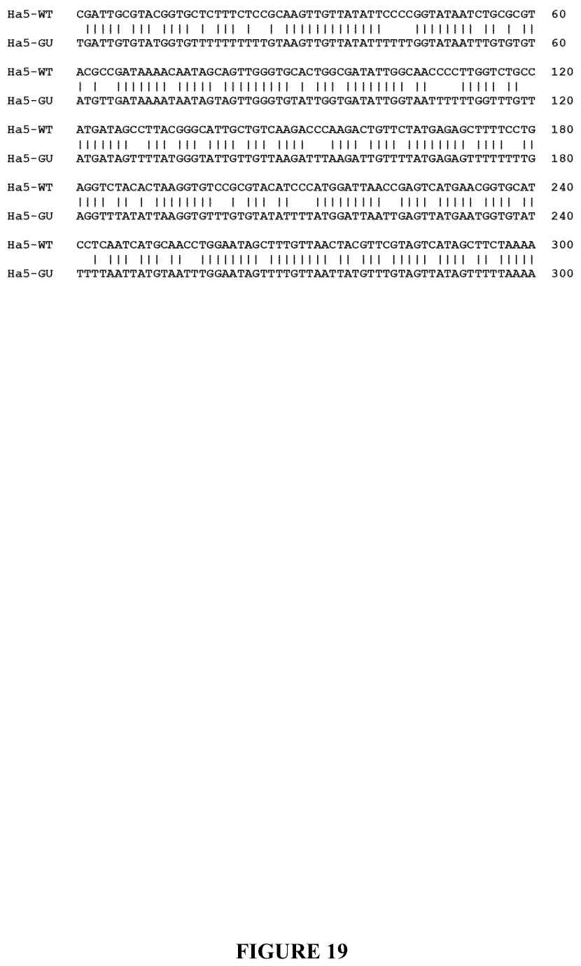

A nucleotide sequence for a cDNA corresponding to the mRNA encoding H. armigera trypsin-like serine protease (designated herein as Ha5) was obtained from Genbank Accession No. EU874846.1, transcript variant X1, provided herein as SEQ ID NO: 46, encoding a polypeptide with the amino acid sequence SEQ ID NO: 45. A target sense sequence of 300 nucleotides was selected (SEQ ID NO: 47), corresponding to nucleotides 621-920 of SEQ ID NO: 46. This sequence has 25.0% C and 24.3% A nucleotides. A modified sense sequence was designed and made by substituting 75 cytidine nucleotides (25%) in SEQ ID NO: 47 for thymidine nucleotides, resulting in SEQ ID NO: 48. An alignment of SEQ ID NO: 47 and SEQ ID NO: 48 shows the position of the substitutions, spread across the 300-nucleotide sense sequence (FIG. 19).

A genetic construct encoding the G:U modified hairpin RNA designated hpHa5[G:U] was designed and synthesised with the order of components 35S promoter-antisense sequence-spacer-sense sequence-transcription terminator/polyadenylation region. A genetic construct encoding the control, conventional hairpin RNA designated hpHa5[Con] was designed and synthesised with the same order of components except with the unmodified sense sequence. The spacer sequence of 150 nucleotides, complementary to nucleotides 471-620 of SEQ ID NO: 46 encoded the loop in both RNA molecules. The nucleotide sequences for the DNA molecules encoding the transcripts are provided as SEQ ID NO: 49 (hpHa5[Con]) and SEQ ID NO: 50 (hpHa5[G:U]), not including 5′ leader and 3′ trailer/polyadenylation sequences.

### Synaptic Vesicle Glycoprotein

One of the protein families involved in neurotransmission in animals is the synaptic vesicle glycoprotein 2 (SV2) family. The SV2 proteins are transmembrane proteins found on secretory vesicles, including synaptic vesicles, which facilitate exocytosis of synaptic vesicles by rendering them responsive to calcium. SV2 proteins are members of the major facilitator superfamily, a large family of membrane transporters expressed widely throughout bacteria, archaea, and eukarya. SV2s have 12 transmembrane domains and cytosolic N- and C-termini. Drosophila species have multiple SV2 family orthologs including the so-called SV2-like proteins. The present inventors are not aware of any reports of down-regulation of the genes encoding SV2 with RNAi.

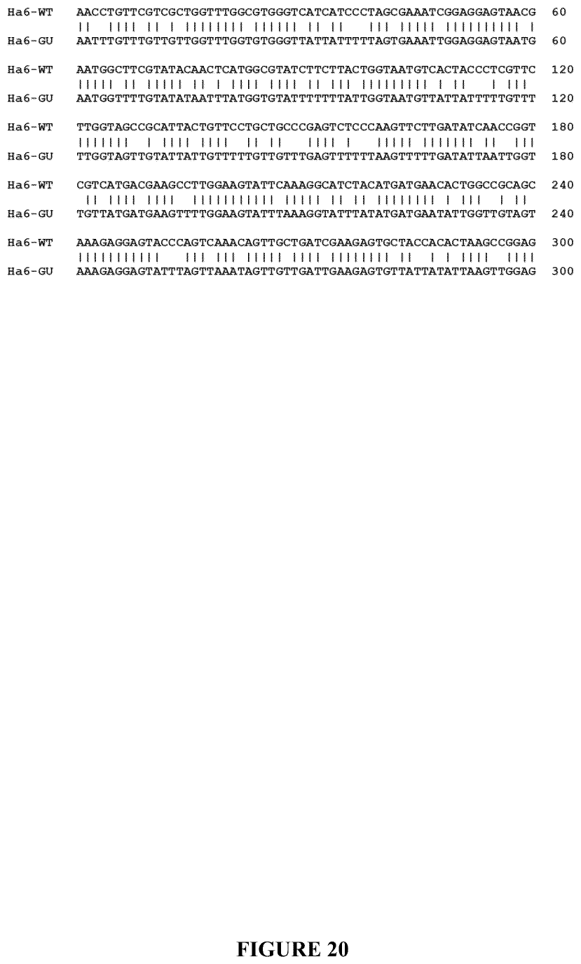

A nucleotide sequence for a cDNA corresponding to the mRNA encoding a H. armigera synaptic vesicle glycoprotein (designated herein as Ha6) was obtained from Genbank Accession No. XP_021181756.1, transcript variant X2, provided herein as SEQ ID NO: 52, encoding a polypeptide with the amino acid sequence SEQ ID NO: 51. A target sense sequence of 300 nucleotides was selected (SEQ ID NO: 53), corresponding to nucleotides 1010-1309 of SEQ ID NO: 52. A modified sense sequence was designed and made by substituting 77 cytidine nucleotides (25.7%) in SEQ ID NO: 53 for thymidine nucleotides, resulting in SEQ ID NO: 54. An alignment of SEQ ID NO: 53 and SEQ ID NO: 54 shows the position of the substitutions, spread across the 300 nucleotide sense sequence (FIG. 20).

A genetic construct encoding the G:U modified hairpin RNA designated hpHa6[G:U] was designed and synthesised with the order of components 35S promoter-antisense sequence-spacer-sense sequence-transcription terminator/polyadenylation region. A genetic construct encoding the control, conventional hairpin RNA designated hpHa6[Con] was designed with the same order of components except with the unmodified sense sequence. The spacer sequence of 150 nucleotides, complementary to nucleotides 1310-1459 of SEQ ID NO: 52 encoded the loop in both RNA molecules. The nucleotide sequences for the DNA molecules encoding the transcripts are provided as SEQ ID NO: 55 (hpHa6[Con]) and SEQ ID NO: 56 (hpHa6[G:U]), not including 5′ leader and 3′ trailer/polyadenylation sequences.

### Troponin C

Troponin is a complex comprised of three subunits designated troponins C, I and T which are important for controlling muscle contraction and relaxation in invertebrates (Eldred et al., 2014). The Troponin C subunit senses changes in the level of calcium, thereby inducing conformational changes that trigger contraction events (Herranz et al., 2004). Troponin C is expressed throughout the insect with the highest levels displayed in the intestinal tract, head and feet (Lan et al., 2018). In the silkworm Bombyx mori, the multiple genes encoding troponin C are expressed in the muscular tissues of the silkworm, including portions of the head, the Malpighian tubule, the body wall and the gut (Chen et al., 2008). In Drosophila, reduced expression of the genes encoding troponin C impacted muscle contraction and ability to fly (Eldred et al., 2014). Larvae of the green rice leafhopper, Nephotettix cincticeps (Homoptera), injected with dsRNA of about 477 basepairs targeting the mRNA encoding troponin C showed a variety of defects impacting fitness, including survival, feeding capacity and weight gain (Lan et al., 2018).

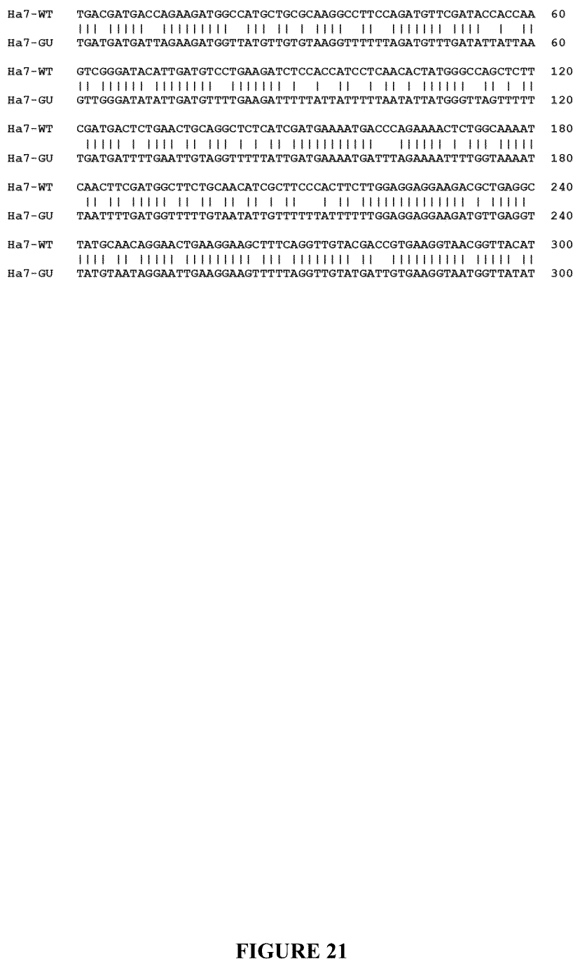

A nucleotide sequence for a cDNA corresponding to the mRNA encoding H. armigera Troponin C (designated herein as Ha7) was obtained from Genbank Accession No. XM_021340134.1, provided herein as SEQ ID NO: 58, encoding a polypeptide with the amino acid sequence SEQ ID NO: 57. A target sense sequence of 300 nucleotides was selected (SEQ ID NO: 59), corresponding to nucleotides 140-439 of SEQ ID NO: 58. This sequence has 25.7% C and 26.0% A nucleotides. This sequence has 25.7% C and 25.0% A nucleotides. A modified sense sequence was designed and made by substituting 77 cytidine nucleotides (25.7%) in SEQ ID NO: 59 for thymidine nucleotides, resulting in SEQ ID NO: 60. An alignment of SEQ ID NO: 59 and SEQ ID NO: 60 shows the position of the substitutions, spread across the 300-nucleotide sense sequence (FIG. 21).

A genetic construct encoding the G:U modified hairpin RNA designated hpHa7[G:U] was designed and synthesised with the order of components 35S promoter-antisense sequence-spacer-sense sequence-transcription terminator/polyadenylation region. A genetic construct encoding the control, conventional hairpin RNA designated hpHa7[Con] was designed with the same order of components except with the unmodified sense sequence. The spacer sequence of 150 nucleotides, complementary to nucleotides 440_587 of SEQ ID NO: 58, encoded the loop in both RNA molecules. The nucleotide sequences for the DNA molecules encoding the transcripts are provided as SEQ ID NO: 61 (hpHa7[Con]) and SEQ ID NO: 62 (hpHa7[G:U]), not including 5′ leader and 3′ trailer/polyadenylation sequences.

### Titin

The protein titin, the largest known protein in vertebrates, is involved in the elasticity and structural integrity of myofibrils and thereby has key structural and mechanical roles in cardiac and skeletal muscle in vertebrates. The human titin gene contains 364 exons over a length of more than 38 kb, coding for a protein of about 4200 kDa (Chauveau et al., 2014). The D-titin gene in Drosophila spans about 110 kb in the genome and encodes a protein of almost 2000 kDa (Machado and Andrew, 2000). The gene is expressed in all striated muscle cells in the insect and the protein localises to chromosomes and to sarcomeres. Titin transcripts are differentially spliced, particularly in regions encoding the tandem-Ig and PEVK domains, giving rise to many isoforms with different extensible properties. Loss of titin protein impacts chromosomal integrity during mitosis whereby mutant embryos display polyploidy, chromosome fragmentation and irregular condensation (Machado and Andrew, 2000). Defects in titin also result in myoblast fusion and a failure for midgut constrictions to form, resulting in insect death.

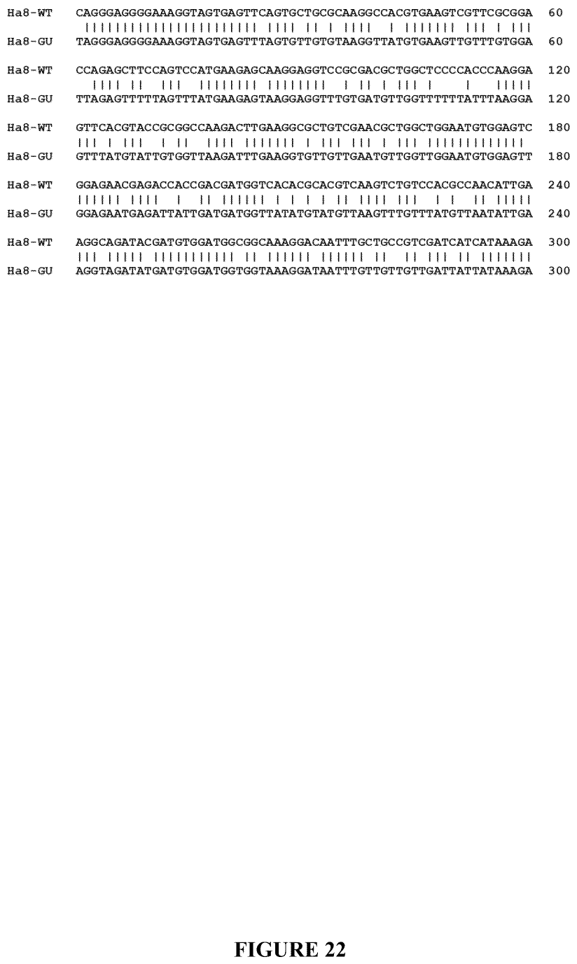

A nucleotide sequence for a cDNA corresponding to the mRNA encoding H. armigera Titin (designated herein as Ha8) was obtained from Genbank Accession No. XM_021340134.1, provided herein as SEQ ID NO: 64, encoding a polypeptide with the amino acid sequence SEQ ID NO: 63. A target sense sequence of 300 nucleotides was selected (SEQ ID NO: 65), corresponding to nucleotides 520-819 of SEQ ID NO: 64. This sequence has 25.7% C and 26.0% A nucleotides. A modified sense sequence was designed and made by substituting 77 cytidine nucleotides (25.7%) in SEQ ID NO: 65 for thymidine nucleotides, resulting in SEQ ID NO: 66. An alignment of SEQ ID NO: 65 and SEQ ID NO: 66 shows the position of the substitutions, spread across the 300-nucleotide sense sequence (FIG. 22).

A genetic construct encoding the G:U modified hairpin RNA designated hpHa8 [G:U] was designed and synthesised with the order of components 35S promoter-antisense sequence-spacer-sense sequence-transcription terminator/polyadenylation region. A genetic construct encoding the control, conventional hairpin RNA designated hpHa8[Con] was designed with the same order of components except with the unmodified sense sequence. The spacer sequence of 150 nucleotides, complementary to nucleotides 821-970 of SEQ ID NO: 64 encoded the loop in both RNA molecules. The nucleotide sequences for the DNA molecules encoding the transcripts are provided as SEQ ID NO: 67 (hpHa8[Con]) and SEQ ID NO: 68 (hpHa8 [G:U]), not including 5′ leader and 3′ trailer/polyadenylation sequences.

### Example 9: Expression of Candidate Target Genes in H. Armigera Larvae

Expression levels of the ten selected candidate target genes, including the PBAN gene (Example 4), were measured by quantitative RT-PCR in untreated H. armigera larvae that were neonates or 1, 3, 5 or 8 days after hatching. These assays used gene specific primers to measure the mRNA levels. Table 2 provides the SEQ ID NOs for the primer pairs for the mRNA for each target gene and the reference gene encoding EF1 in the RT-PCR assay. The expression level for each target gene was thereby normalised to the expression of the EF1 gene, a constitutively expressed gene used here as a reference gene.

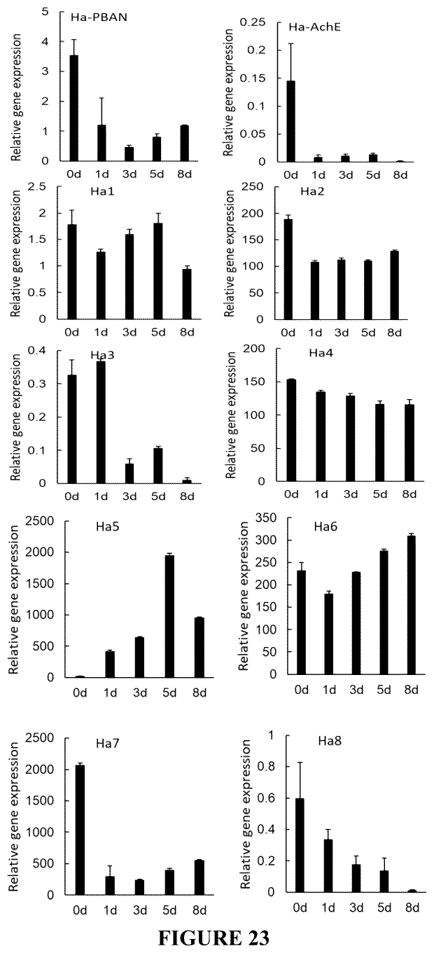

RNA was extracted from the larvae at each stage of development using TRIzol Reagent (Invitrogen, Catalog number 15596026) following the manufacturer's instructions. In each RT-PCR assay, 1 μg of total larval RNA was treated with RQ1 RNase-free DNase (Promega, Catalog number M6101) following the manufacturer's instructions to remove any DNA from the RNA preparations. The DNase-treated RNA was reverse-transcribed with SuperScript III First-Strand Synthesis System (ThermoFisher, Catalog number 18080051) using oligo (dT) 23V (V=A, C, G mixed nucleotides) as the first strand primer. Real-time PCR was performed using each primer pair in three technical triplicates for 40 cycles with Fast SYBR Green Master Mix (ThermoFisher, Catalog number 4385612) following the manufacturer's instructions. Results were analysed using the Delta-Delta Ct method. The results of the expression analyses are shown in FIG. 23.

TABLE 2
Oligonucleotide primers used to measure
expression levels by RT-PCR.

| | Primer name | SEQ |
|---|---|---|
| Gene | (F forward, R reverse) | ID NO |
| PBAN | Pban-qPCR-F1 | 69 |
| | Pban-qPCR-R1 | 70 |
| AchE | Ha_AchE_qRT-PCR-F | 71 |
| | Ha_AchE_qRT-PCR-R | 72 |
| Ha1 (EcR) | Ha_EcR_qRT-PCR-F | 73 |
| | Ha_EcR_qRT-PCR-R | 74 |
| Ha2 (Coatomer beta) | Ha_Coβ_qRT-PCR-F | 75 |
| | Ha_Coβ_qRT-PCR-R | 76 |
| Ha3 (HR3) | Ha_HR3_qRT-PCR-F | 77 |
| | Ha_HR3_qRT-PCR-R | 78 |
| Ha4 (v-ATPase) | Ha_V-ATPase_qRT-PCR-F | 79 |
| | Ha_V-ATPase_qRT-PCR-R | 80 |
| Ha5 (Trypsin-like | Ha_Try-Ser-Protease_qRT-PCR-F | 81 |
| serine protease) | | |
| | Ha_Try-Ser-Protease_qRT-PCR-R | 82 |
| Ha6 (Synaptic vesicle | Ha_Syn-Ves_qRT-PCR-F | 83 |
| glycoprotein) | | |
| | Ha_Syn-Ves_qRT-PCR-R | 84 |
| Ha7 (Troponin C) | Ha_TroponinC_qRT-PCR-F | 85 |
| | Ha_TroponinC_qRT-PCR-R | 86 |
| Ha8 (Titin) | Ha_Titin_qRT-PCR-F | 87 |
| | Ha_Titin_qRT-PCR-R | 88 |
| EF1 reference gene | CB_qPCR_EF1_F | 89 |
| (control) | | |
| | CB_qPCR_EF1_R | 90 |

The ten selected target genes could be classified into three groups based on their expression levels and further grouped for changes in expression during the time period. A first group of PBAN, AchE, Ha1, Ha3 and Ha8 showed low expression relative to the reference gene at each stage of development, as can be seen from the y-axis in FIG. 23. A second group of Ha2, Ha4 and Ha6 showed moderate expression levels while a third group of Ha5 and Ha7 showed relatively high expression level compared to the others, all being normalised to the constitutive reference gene. Among the ten genes, PBAN, AchE, Ha3, Ha7 and Ha8 showed relatively higher expression levels at the newly hatched stage which then declined in the days after hatching. In contrast, target gene Ha5 showed a relatively low expression level in the neonates and then gradually increased expression during the next 8 days. Three of the selected target genes, Ha2, Ha4 and Ha6, were expressed quite evenly during the 8 days after hatching, within the accuracy of the quantitative RT-PCR method, and were considered by the inventors to be constitutively expressed within the examined time period.

### Example 10: Development of Rapid Feeding Assays

In Vitro Feeding Tests with Hairpin RNA Using Artificial Media

The genetic constructs designated hpAChE[Con] and hpAChE[G:U] (Example 8) were synthesised with flanking XhoI and HindIII restriction enzyme sites and, using these, inserted between the XhoI and HindIII sites of the expression vector pART7. The expression cassettes were excised from there and inserted into the T-DNA region of the binary vector pART27 (Gleave, 1992) to form the p35S::hpAchE[Con]::Ocs-T and p35S::hpAchE [G:U]::Ocs-T genetic constructs for plant transformation. Plasmid DNAs of the pART7 constructs linearised with HindIII were used as templates for in vitro transcription using the TranscriptAid T7 High Yield Transcription Kit (Thermo Scientific, Catalog No. K0441) following the manufacturer's instructions to produce the corresponding hairpin RNAs. Feeding assays were carried out by incorporating the hpAChE[Con] or hpAChE[G:U] transcripts at 400 μg/ml in 300 μl artificial diet (Example 1) per well in a 96-well plate. This experiment did not use any form of packaging or protection of the RNA transcripts. Two neonates, hatched not more than 10 hours previously, were put into each well and the plates incubated for two or four days during which time the larvae fed on the artificial diet material. Wells containing the artificial diet without hairpin RNA were prepared as controls, either with or without added hairpin RNA. After the 2- or 4-day incubation, the leftover artificial diet material was collected from each well. RNA was extracted from each material using TRIzol™ Reagent following the manufacturer's instructions. The extracted RNA was electrophoresed on gels and examined by Northern blotting using a radiolabelled probe corresponding to the complement of hpAChE loop, to look for integrity of the RNA transcripts.

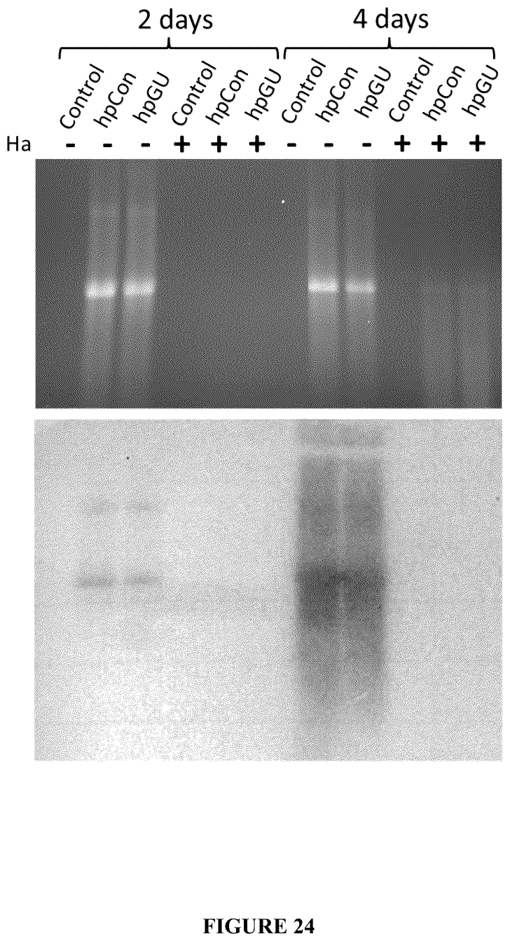

FIG. 24 shows the results of this experiment. The upper panel shows the presence of RNA molecules by staining, and the lower panel shows the Northern blot after probing with the radiolabelled probe. As seen in both panels, the RNA transcripts were detected as full-length molecules when incorporated into the artificial diet after 2 and 4 days in the absence of the larvae, but no transcript was detected when the H. armigera larvae were present. From these observations, it was concluded that the hpRNA molecules were degraded in the presence of the cotton bollworm larvae, even after a short period of 2 days, probably by RNAse activity coming from the larvae, for example in exudates. The inventors concluded that this system of supplementing the artificial diet in this format with the unprotected hairpin RNA transcripts was not a suitable system for testing the efficacy of the hairpin RNAs by oral delivery to the larvae, and that a different system needed to be established.

In a similar experiment, an analogous pair of genetic constructs designated hpPBAN[Con] and hpPBAN[G:U] (Example 4) were transcribed in either bacterial or yeast cells. The E. coli strain HT115 which is deficient in RNAse III was used as the bacterial host, and S. cerevisiae strain HF7C was used for the yeast host cells. The gene encoding the hpPBAN RNAs in the yeast cells was expressed under the control of the S. cerevisiae ADH1 promoter, a promoter of moderate strength when fully induced, and the polyadenylation region/transcription terminator from the ADH1 gene (Zhong et al., 2019). The whole cells were incorporated into the artificial diet without extracting the RNA, in an attempt to protect the RNA transcripts. Only 2 or 3 out of 16 H. armigera larvae died when fed with the hpPBAN[GU] producing cells, compare to no death or 1 dead larva when fed with the hpPBAN[Con] producing cells. Since the results for mortality were modest and other delivery means proved more successful (below), no molecular analysis was done on the fate of the RNA transcripts. Increasing the dosage of the RNA producing cells in the diets, for example by using expression systems with stronger promoters, would likely increase the mortality rate. Experiments are conducted to test this.

### Painted Leaf Assays

An alternative delivery method that was considered and tested was a painted leaf assay where RNA transcripts were applied to leaves of different plant species and provided to H. armigera larvae as their food source. Such assays more closely imitate the natural situation for lepidopteran pests such as H. armigera, which naturally feeds on a wide range of plant species. To identify a plant species which was favourable for H. armigera as a food source, leaves were obtained from three different species of plants growing in a local garden, namely pakchoi (Brassica rapa subsp. chinensis), kale (Brassica oleracea) and napa cabbage (Brassica rapa subsp. pekinensis). Newly hatched H. armigera neonates were applied onto the detached leaves from these plants, without added RNA. The leaves were then maintained on wet filter paper in petri dishes at 25° C. in a rearing room so the leaves would not dry out during the experiment. It was observed that, of the species tested, the pakchoi leaves were the preferred food source for the larvae.

Hairpin RNA was prepared by in vitro transcription from the constructs designated hpPBAN[Con] and hpPBAN[G:U] (Example 8) using the same method as described above for hpAChE[Con] and hpAChE[G:U]. The RNAs were suspended at a concentration of 200 μg/ml in a buffer made up with DEPC-treated water containing 0.5 mM sodium EDTA and 0.01% (v/v) of the surfactant Silwet-L77 (PhytoTech Labs, Catalog No. S7777). Using a paintbrush, each detached pakchoi leaf was brushed on both sides with the solution, covering the entire leaf surfaces, and the brushed leaves allowed to air dry. Control leaves were painted with the same buffer solution but lacking the hpRNA. After drying, the leaves were placed on wet filter paper in petri dishes to avoid further drying out. Twenty to thirty newly hatched larvae, not more than 10 hr after hatching, were carefully placed onto each coated leaf using a paintbrush. The petri dishes were sealed with micropore tape and then kept in a rearing room at 25±1° C., 50±10% relative humidity, with 14 hr light and 10 hr dark per day to imitate natural light conditions. Leaves without applied larvae, painted with either the RNA solution or the control (no RNA) solution, were used as negative controls.

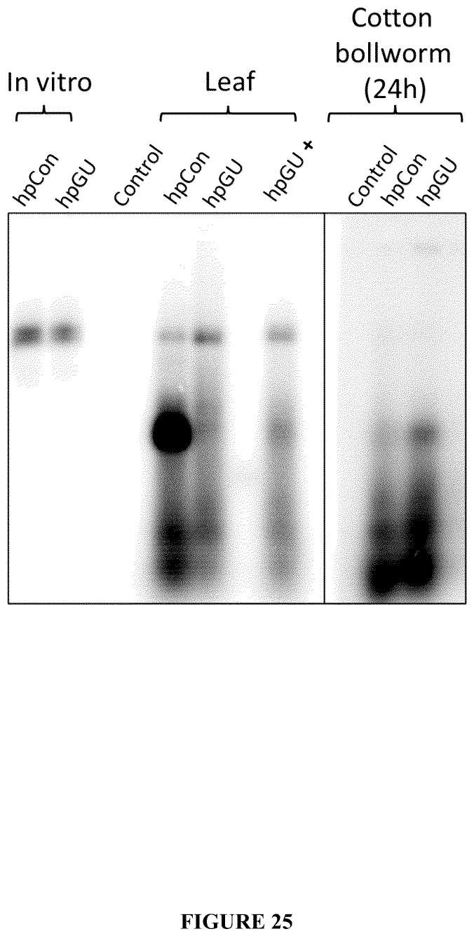

Samples from the leaves and from the larvae were collected after 24 hr and RNA extracted from each sample. These RNAs were analysed by Northern blot using a radiolabelled probe corresponding to the antisense sequence in the hpRNA and the complement of the pAChE loop. The results of the Northern blot are shown in FIG. 25.

Strong hybridising signals were detected from leaves coated with the hpPBAN[Con] and hpPBAN[GU] leaves which had not had larvae applied. These signals included a relatively faint band corresponding to the full-length hpRNAs and a more intense smear for RNA fragments which indicated that processing or degradation of the hpRNA had occurred on or in the coated leaves. These signals were also observed for leaves that had been painted with the hpPBAN[GU] RNA and had larvae applied (GU+lane). The lane for the hpPBAN[Con] lane also had an intense band of a size that corresponded to the loop sequence, suggesting that this hpRNA was being processed in a specific manner to produce a circularised loop as observed in bacterial cells. That band was not present in the lanes for hpPBAN[G:U], suggesting that hpPBAN[G:U] RNA was reacting differently when applied on the plant leaf. It was concluded that the neonate larvae had not affected the hpPBan[G:U] RNA that had been applied to the pakchoi leaves beyond the processing or degradation observed in the absence of the larvae, perhaps because the RNA had been taken up into the leaf cells or into the air spaces within the spongy mesophyll tissues of the leaves.

RNA was also extracted from the larvae that had been applied to the leaves. The Northern blot for that RNA showed a smear of hybridising signals to the probe, for both the hpPBAN[Con] and hpPBAN[GU] RNAs. These were shorter than full length hpRNA. It was concluded that the larvae had clearly ingested the hpRNAs which were detected as hybridising to the probe, and that some degradation, but not complete breakdown, had occurred in the insect larvae.

Attempts were made to detect small RNA products in the larvae produced from the hpRNAs, but no specific hybridising bands were observed. This was likely due to the rapid turnover of small RNAs in the larvae.

Based on these observations, it was concluded that the painted leaf method could be used to test the hpRNA[G:U] molecules for their efficacy and compare them to the corresponding hpRNA[Con] molecules when ingested by the larvae, in an assay that approximated the natural system for the insect pest.

### Example 11: Reduction of Gene Expression in the Painted Leaf Assay

To test the hpRNAs for reduction of target gene expression in larvae in a feeding assay, five of the G:U hairpin RNAs, each targeting different H. armigera genes, were painted onto pakchoi leaves as in Example 10. Neonate larvae were applied to the painted leaves on day zero, and then transferred to fresh, painted leaves on days 1, 3 and 5. Some of the larvae were collected on day 3 and some on day 6 and RNA extracted for analysis. The applied RNAs were for hpHA1[G:U], hpHA2[G:U], hpHA3[G:U], hpHA4[G:U] and, in a second experiment, hpHA5[G:U] and a repeat of hpHA1[G:U]. A range of hpRNA concentrations at between 1-500 ng of hpRNA per 0.5 ml per leaf was painted on the leaves. The level of the target RNA was measured in each RNA sample by RT-PCR using the methods as described in Example 1, for the extracted RNAs after 3 days or 6 days feeding. The level for each target gene was normalised to the level of expression of the EF1 gene as a reference gene, using the primers described in Example 8.

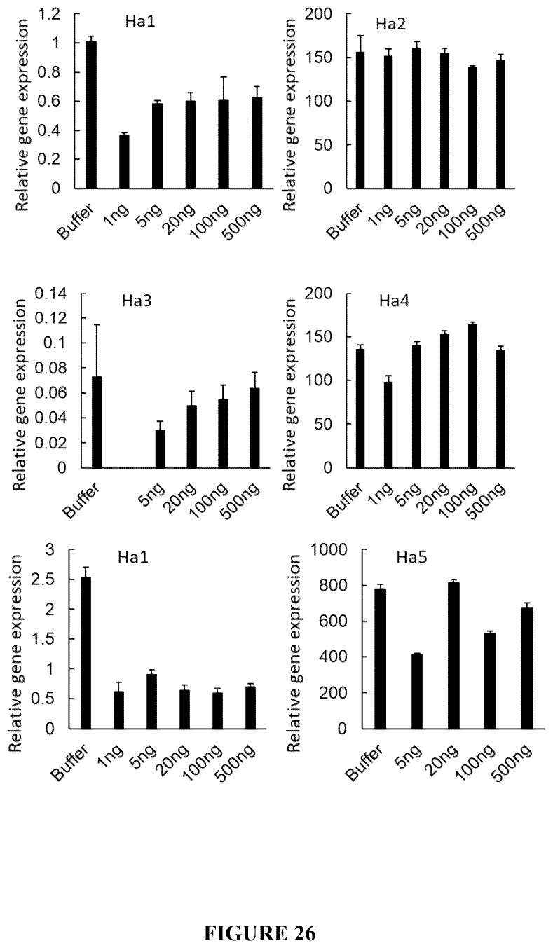

The results are shown in FIG. 26. In the first experiment (FIG. 26 upper panel), the level of Ha1 target gene transcript was reduced by 37-63% relative to the buffer control, even for the lowest dose of hpHa1[G:U] RNA applied to the leaves. The level of both of the Ha3 and Ha4 transcripts was reduced in one sample each, but not consistently in other samples. The level of transcript for Ha2 did not appear to be reduced in any of the samples. In the second experiment (FIG. 26 lower panel), where the larvae were allowed to feed for six days, the level of the Ha1 transcript was reduced across all hpRNA doses as in the first experiment, this time showing a reduction of 64-76% relative to the buffer control. The level of the Ha5 transcript was reduced in some samples, by 47% or 32%. These two target genes were selected for further evaluation along with Ha6, based on experiments described in Example 12.

The experiments are repeated for all ten target genes with inclusion of the corresponding conventional, canonically basepaired hpRNA for comparison. Other parameters are measured for the treated larvae, including length, weight and mortality.

The reduction in expression of the Ha1 target gene, which is naturally expressed at a low level compared to most of the tested target genes, was observed for all of the concentrations of hpHa1[G:U] tested, even at Ing RNA/0.5 ml per leaf. It was considered possible that even the lowest dose tested was enough to trigger the inactivation of the target RNA molecules for such a lowly-expressed gene.

### Example 12: Reduction of Gene Expression in the Transient Expression Assay

### In Vivo Leaf Expression System for Insect Feeding Assays

Example 7 describes the development of a rapid, transient expression system for G:U hairpin RNA molecules, using Agrobacterium to deliver genes to express the hairpin RNAs in leaf cells. Those experiments showed that the hpRNA[G:U] constructs resulted in much greater accumulation of full-length transcripts compared to the corresponding hpPBAN[Con] construct, as well as some shorter RNA molecules. Since H. armigera larvae are able to feed on N. benthamiana leaf tissues and to grow until at least the 5th instar, this expression system was used to test the effect of several hpPBAN[Con] and hpPBAN[G:U] RNAs on H. armigera larvae.

In a first experiment, A. tumefaciens cells transformed with the genetic constructs in binary vectors were infiltrated into N. benthamiana leaves to express the constructs and produce the hpRNAs in planta. The infiltrated constructs in this experiment encoded hpHa1[G:U], hpHa2[G:U], hpHa3[G:U], hpHa4[G:U], hpHa5[G:U] and hpHa6[G:U]. Separately, constructs encoding the conventional hairpin RNAs hpHa1[Con] and hpHa5[Con] were infiltrated for comparison with the corresponding G:U hairpin. A hpGUS[G:U] construct was also infiltrated as a negative control, being unrelated to any target gene transcript in the H. armigera larvae. All of the infiltration mixtures also contained A. tumefaciens cells with the genetic construct expressing the CMV 2b silencing suppressor and a GFP construct for showing competence of the plant leaves for expression of transgenes. Each hpRNA construct in the mixtures was infiltrated at a final OD600 of 0.6, the 2b silencing suppressor at a final OD of 0.3 and the GFP construct at a final OD600 of 0.2.

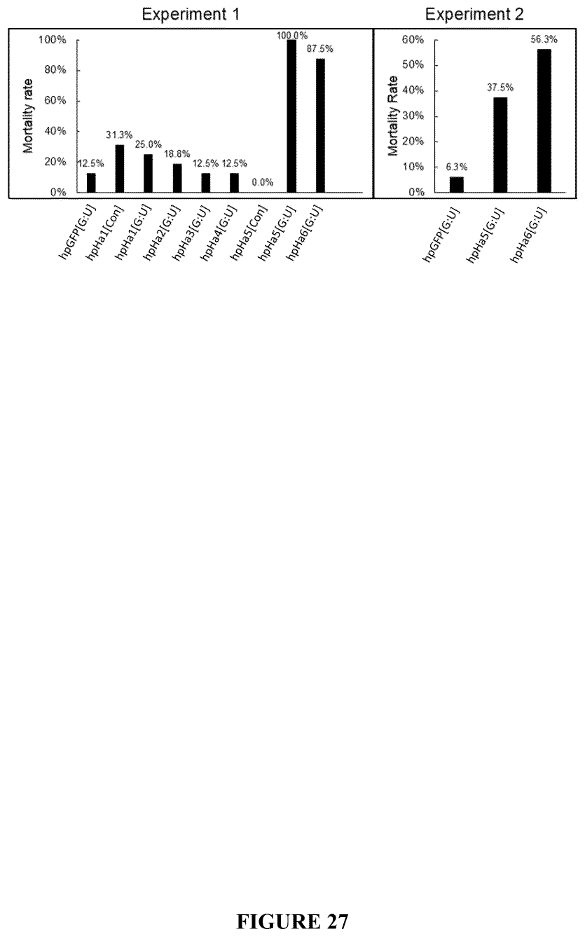

Leaves displaying GFP fluorescence were collected for the feeding assays 2-4 days after agro-infiltration and cut into pieces to fit into 32-well trays. Preliminary experiments had shown that the hpRNAs were produced at high levels in the time period 2-4 days post-infiltration. Third instar larvae were applied to the leaf pieces, one per well, with 16 wells/larva in total per hpRNA. This experiment did not use newly hatched neonates as they had high background mortality rates on N. benthamiana leaves, whereas third instar larvae that had been previously fed on the artificial rearing diet (Example 1) were able to establish feeding on the N. benthamiana leaves and had low mortality on the wild-type leaves. The leaf pieces were replaced with freshly harvested agro-infiltrated leaf pieces every third day. The trays were sealed with a plastic cover and kept in the rearing room at 25±1° C., 50±10% relative humidity, and 14 hr light and 10 hr dark cycles to imitate natural light. Mortality rates were recorded 7 days after the larvae began feeding on the agro-infiltrated leaves. The results are shown in FIG. 27.

It was observed that the mortality rate for the hpHa5[G:U] at 7 days was 100% whereas the corresponding hpHa5[Con] yielded zero mortality, showing a clear and dramatic difference between the conventional hairpin RNA and the G:U modified version. The hpHa6[G:U] also yielded 87% mortality, but there was no comparison possible in this instance since hpHa6[Con] was not tested in this experiment. In contrast, hpHa1[G:U], hpHa2[G:U] and hpHa3[G:U] may have given slightly higher larval mortality but the effect was modest and there was no comparison with the corresponding hpRNA[Con] RNAs in this experiment. The negative control hpGUS[G:U] gave only low mortality in this experiment.

A second experiment was carried out where hpHA1[G:U] and hpHa5[G:U] were tested again in comparison with the negative control hpGUS[G:U]. This time the mortality rate with the modified hairpin RNAs was lower at 37.5% or 56.3% but again much higher than the control.

It was concluded that at least three of the G:U modified hairpins were able to kill the H. armigera larvae at an increased rate, namely when targeting the Ha1, Ha5 and Ha6 gene transcripts, although only the hpHa5[G:U] RNA had a direct comparison with the corresponding, conventional (fully canonical basepaired) hairpin RNA.

Further experiments are conducted in similar fashion against all ten target genes using these constructs in N. benthamiana leaves and with the corresponding conventional hairpin RNAs. Mortality is assessed daily from day 2 to day 8 as well as measurement of the length, weight and progression through developmental stages of the larvae, as well as scores for health.

### Example 13: Generation of Transgenic Plants Expressing Modified Hairpin RNAs

### Generation of Transgenic Tobacco Plants

The genetic constructs hpHa1[G:U], hpHa2[G:U], hpHa3[G:U], hpHa4[G:U], hpHa5[G:U], hpHa6[G:U], hpHa7[G:U] and hpHa8 [G:U] and control constructs hpHa1[Con], hpHa5[Con] and hpHa6[Con] were used to produce transgenic tobacco plants from cultivar W38. In addition, a hpGFP[G:U] construct was used to transform W38 tobacco for use as a negative control to check for non-specific effects of expressing a G:U hairpin RNA on the larvae. A large number (30˜100) of independent TO transgenic lines were generated using Agrobacterium tumefaciens-mediated leaf disk transformation as described in Example 1, with kanamycin (50 mg/L) in the media as the selective agent for shoot regeneration and rooting. Four separate transformation experiments were performed. All 7 constructs except for the hpHa7[G:U] and hpHa6[Con] were used in the first transformation experiment. Each of hpHa1[Con], hpHa1[Con], hpHa1[G:U], hpHa6[Con] and hpHa6[G:U] constructs were used in the second and third transformation experiments, and hpHa7[G:U] in the fourth. Successful root formation in the presence of 50 mg/L kanamycin in the tissue culture medium normally ensured that the plants were transgenic.

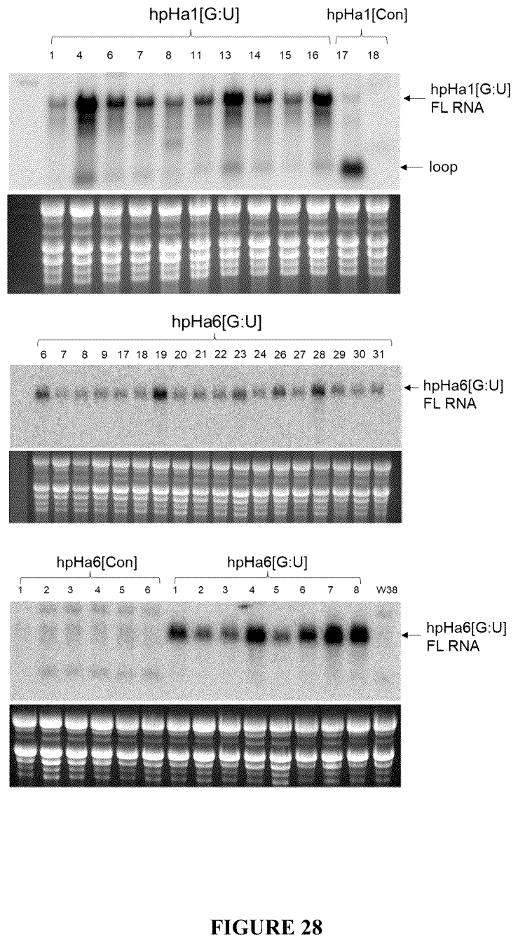

The presence of the transgenes was further confirmed by Northern blot detection of hpRNA transcripts in all hpHa1[G:U] and hpHa6[G:U] transgenic plant lines analyzed. Representative Northern blots are shown in FIG. 28. A clear difference was observed in the hybridising RNAs between the transgenic plants expressing the G:U modified hairpin RNAs and the corresponding conventional hairpin RNAs, with the former plants accumulating substantial amounts of RNA that, from the band mobility, appeared to be full length or close to it. Many of hpHa1[Con] plants exhibited an intense band that hybridised to the probe and had the size expected of a loop fragment. The hpHa6[Con] plants showed only a weaker band corresponding in size with the loop. The conventional hairpin constructs did not show strong bands for the full-length hairpin RNAs, from which it was concluded that the G:U modified hairpin constructs accumulated much greater amounts of full-length transcript. A few hpHa1[Con] and hpHa6[Con] lines showed no or low levels of RNA transcripts, presumably due to the self-induced transcriptional silencing that tends to occur with the fully canonically base-paired hpRNA transgenes (Zhang et al., 2022). For insect feeding assay and molecular analysis, 25˜85 TO plants were randomly selected and planted in soil. These plants were maintained in a 25° C. growth room with 14 hr light/10 hr darkness cycles for continued growth.

These genetic constructs are also used to generate transgenic cotton plants using standard methods, using explants from cotton variety Coker 315 (Table 3).

TABLE 3
Number of independent T0 N. tabacum
W38 transgenic plants produced

| | | Number of transgenic |
|---|---|---|
| | Number of transgenic | plants used in insect |
| Constructs | plants planted in soil | feeding experiments |
| hpHa1[WT] | 79 | 24 |
| hpHa1[G:U] | 70 | 41 |
| hpHa2[G:U] | 25 | 21 |
| hpHa3[G:U] | 25 | 18 |
| hpHa4[G:U] | 25 | 21 |
| hpHa5[WT] | 35 | 12 |
| hpHa5[G:U] | 35 | 33 |
| hpHa6[WT] | 49 | 18 |
| hpHa6[G:U] | 85 | 60 |
| hpHa8[G:U] | 25 | 15 |
| hpGFP[G:U] | 25 | 16 |

Transgenic cotton plants producing hpHa6[G:U] were produced as described, and expression of the hpHa6[G:U] was confirmed.

### Example 14: Feeding Tests on Transgenic Tobacco Plants

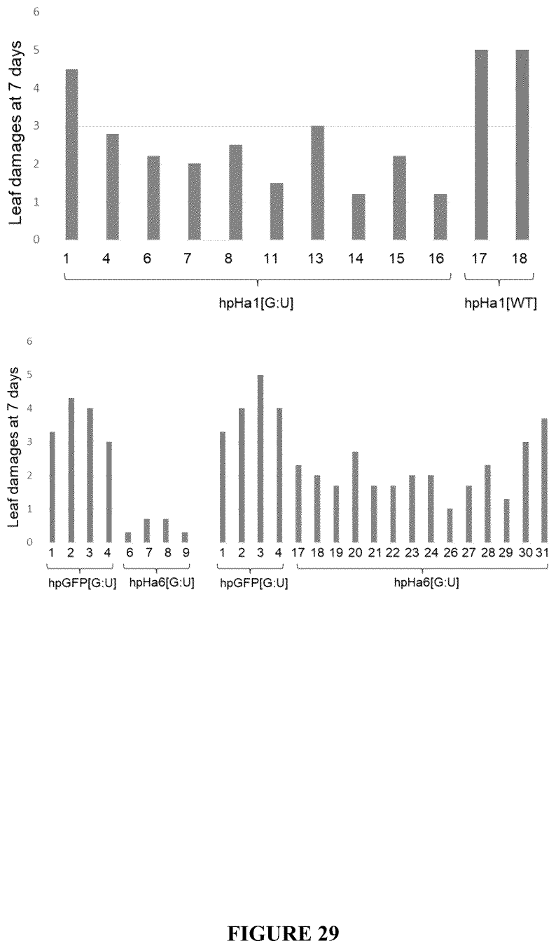

For initial insect feeding tests, 2-4 leaves of 4-8 week old transgenic plants, grown under normal light intensity in the first three weeks after being transferred into soil and then under 50% light intensity, were excised from the plants, cut into 2-4 sections for each leaf, and placed on water-agar in petri dishes to provide 2-4 replicates for each transgenic plant. Two or three H. armigera larvae at the 2-3 instar stage were placed on the leaf sections and the dishes sealed with micropore tape and kept in a 25° C. insect rearing room. The dishes were covered with Miracloth to reduce light. The amount of leaf damage was scored on days 4 and 7 with a damage rating from 1 to 5, with 5 being the maximum amount of damage, and the leaf pieces photographed after 7-10 days of larval feeding. Among the plants from the different constructs, those expressing the hpHa1[G:U] and hpHa6[G:U] RNAs displayed clearly reduced leaf damage after 7-10 days of feeding compared to the plants expressing the corresponding conventional hairpin RNA (FIG. 29).

Whole plant feeding assays were also carried out. Individual T0 transgenic plants growing in soil were placed inside two layers of porous plastic bags. Six 2nd instar larvae were placed onto each plant and the bags sealed and maintained in the insect rearing room. After 10 days, the bags were opened and the amount of insect damage recorded. No live larvae were found on the resistant hpHa6[G:U] plants when first checked on day 10, whereas living larvae were actively feeding on the control plants. The plants transformed with the hpHa6[G:U] RNA construct clearly showed less insect damage than the control plants, and the larval size on G:U plants was also significantly reduced. The bags were re-sealed and maintained until day 18. When re-opened on day 18, the difference between the hpHa6[G:U] and control plants was even more stark, with the former plants showing much less damage.

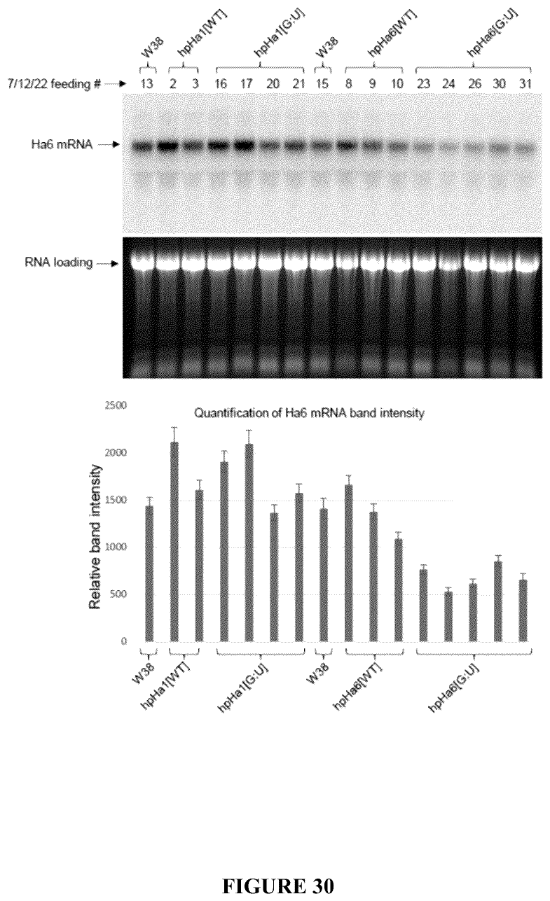

The enhanced trans-kingdom RNAi effect of the hpHa6[G:U] RNA against the Ha6 gene in H. armigera larvae was confirmed by Northern blot hybridisation analysis of Ha6 mRNA in insects that fed on the TO transgenic tobacco leaves. As shown in FIG. 30, Ha6 mRNA levels were clearly downregulated in the insects fed on leaves of the five hpHa6[G:U] plants compared to the wild-type N. tabacum (W38) and the hpHa1 transgenic plants. Insects from two of the three hpHa6[Con] plants also showed Ha6 mRNA downregulation, but at a much lower degree compared to the hpHa6[G:U] plants.

Seed were collected from TO transgenic plants expressing hpHa6[G:U], hpHa7[G:U] or hpHaPBAN[G:U] (Example 6) RNAs and the corresponding conventional hairpin RNAs and plated on ½ MS medium containing kanamycin to select for T1 transgenic progeny plants. Seedlings were transferred to soil grown in a growth room for another 3-4 weeks. Each independent line was represented by 2-3 pots. Ten early 2nd instar larvae were placed onto the plants in each pot, which were then covered with transparent plastic lids and kept in the growth room at 25-27° C. The T1 transgenic plants expressing the hpHa6[G:U], hpHa7[G:U] or hpHaPBAN[G:U] constructs exhibited significantly less insect damage compared to transgenic plants expressing the hpHa6[Con], hpHa7[Con] or hpHaPBAN[Con] constructs, respectively, or the control hpGFP[G:U] construct after 10 days of feeding. Increased larval mortality was also observed for the plants expressing the G:U modified hairpin RNAs compared to the conventional hairpins.

These results indicated that at least Ha6, Ha7 and HaPBAN were effective target genes for H. armigera, and that the G:U hpRNA design was superior to the conventional hpRNA design in conferring trans-kingdom RNAi against H. armigera.

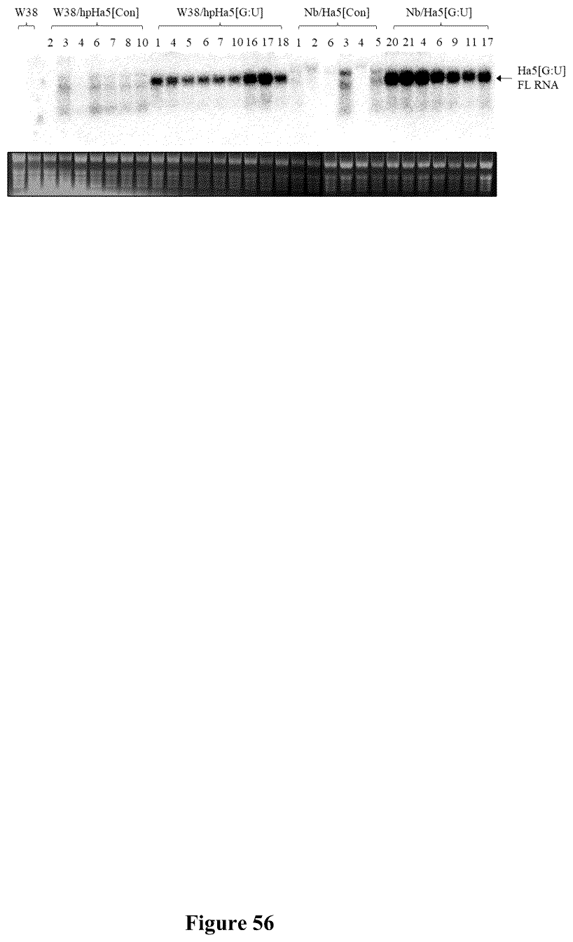

In similar fashion, seed were collected from TO transgenic plants having the hpHa5[G:U] and hpHa5[Con] constructs and transgenic T1 progeny were selected on medium containing kanamycin. The T1 seedlings with true leaves and well-developed roots were then subcultured onto non-selective ½ MS medium. Two plates were prepared for each individual line, one for northern blot analysis and one for an insect feeding assay. Leaf tissues from approximately 10 T1 seedlings were collected 3-4 weeks after germination to represent each individual line. Northern blot analyses were conducted to examine the patterns and levels of hpHa5[Con] or hpHa5[G:U] transgene expression. Abundant full-length hpRNA was detected in all tested transgenic plants expressing the G:U modified RNA molecules, whereas significantly weaker hybridisation signals and processed loop fragments were observed in transgenic plants transformed with the hpHa5[Con] construct (FIG. 56). Some hpHa5[Con] plants showed no or low levels of RNA transcripts, similar to that observed in hpHa1[Con] and hpHa6[Con] plants.

The transgenic T1 seedlings transformed with hpHa5[Con] or hpHa5[G:U] from the insect feeding assay plate were transferred to soil and grown in a growth room for another 3-4 weeks. Each independent line was represented by 2-3 pots, with 3-4 seedlings per pot. Ten early 2nd instar larvae were placed onto each pot, which were then covered with transparent plastic lids and kept in the growth room at 25-27° C. The plants from two hpHa5[Con] lines (#6 and #7) and one hpHa5[G:U] line (#18) consistently showed resistance to H. armigera compared to other lines or untransformed W38 plants after 15 days of feeding. These results indicated that Ha5 was also an effective target gene for H. armigera and that the G:U-modified hairpin RNA was able to confer trans-kingdom RNAi against H. armigera.

The hpHa5[Con] and hpHa5[G:U] constructs were also used to transform N. benthamiana plants. Transgenic T1 plants were selected and analysed by Northern blot hybridisation using a probe for the RNA molecules. As for the tobacco plants, abundant full-length hpRNA was detected in the transgenic Nb plants expressing the G:U-modified RNA molecule, whereas significantly weaker hybridisation signals and processed loop fragments were observed in transgenic plants transformed with the hpHa5[Con] construct (FIG. 56). Since N. benthamiana plants are not good hosts for H. armigera, these plants were not tested for resistance to the insects.

### Example 15: Design of Vectors for Increasing Expression of Hairpin RNA in Plant Cells

### Introduction

Previous experiments described herein used the enhanced 35S (e35S) promoter from CaMV and an ocs gene polyadenylation region/transcription terminator (3′-Ocs) to drive hairpin gene expression in the plant cells. This combination of expression elements provided high levels of RNA production in leaves, such as in Nicotiana plants. However, the present inventors sought further enhancement of hairpin RNA expression in plant cells and therefore tried the use of replicating viral vectors, in this instance vectors based on Gemini Virus (GV) genomes. These were designed to increase expression levels by multiplying the copy number of a gene of interest within the plant cells. The present inventors therefore tested GV vectors for hairpin RNA production, adapting the vectors to a format that was compatible with the GoldenGate cloning system. This modular cloning approach allowed rapid interchange and testing of elements for expression and for different targeting sequences in the hairpin RNAs.

### Design and Construction of Replicative Expression Vectors Adapted for GoldenGate Cloning

Gemini Virus (GV) replicative expression vectors were designed and made based on the structure of a LSL Bean Yellow Dwarf Virus (BeYDV) plasmid vector (Baltes et al., 2014), as follows. The virus BeYDV has a single-stranded DNA genome and has a relatively wide host range among dicotyledonous plants. Vectors based on its replicative regions are therefore expected to replicate in many dicotyledonous plants, including N. benthamiana used as described herein. The LSL vector includes the large intergenic region (LIR) and short intergenic region (SIR) required for viral replication as well as a gene encoding the trans-acting replication (Rep/RepA) proteins but does not have the genes encoding the viral coat protein and the movement protein required for cell to cell spread of the wild-type virus. Those genes were deleted to provide space for inserting heterologous genes.

To adapt the LSL-BeYDV vector to be suitable for type IIS restriction enzyme cloning in the GoldenGate system for the construction of L1 modules, a guanosine at nucleotide position 1,119 with reference to SEQ ID NO: 93, within the Rep/RepA coding region, was replaced with an adenine to remove a BsaI site that otherwise would interfere with subsequent cloning, providing the nucleic acid having SEQ ID NO: 93. This substitution did not change the encoded Rep amino acid sequence and, as shown afterward, did not adversely affect Rep function. This vector backbone is referred to herein with the notation “GV”. Further modifications were made where a thymidine at position 1,469 in the Rep 5′UTR sequence, with reference to SEQ ID NO: 93, was additionally changed to either a guanosine as per Diamos and Mason (2019) in the GVc vector, or an adenosine in the GVt vector. The complementary nucleotides C or T in the sense strand are less favoured for translation initiation in their context 3 basepairs upstream of the ATG start codon. This was done with the aim of decreasing translation of the Rep protein to reduce cell necrosis that tended to occur with GV vectors. Both vectors were designed to be assembled from BsaI-ended DNA fragments as modules and this strategy thereby provided flexibility and adaptability as for the GoldenGate cloning system, allowing various combinations of modules and switching of any one module for another.

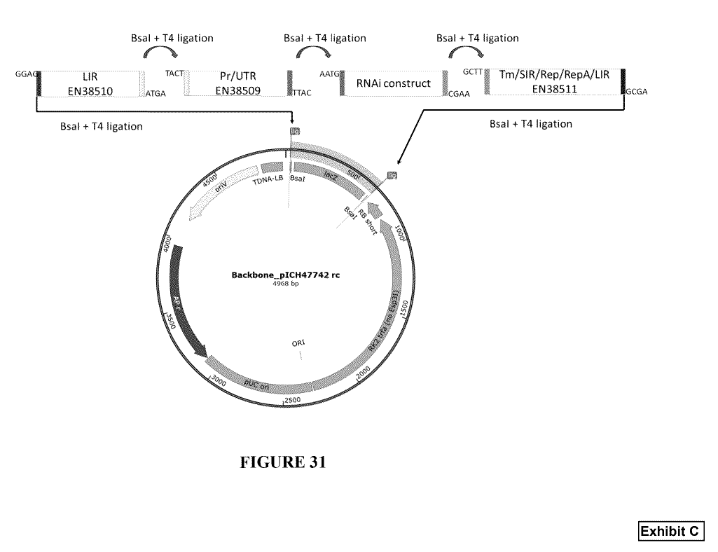

DNA fragments for the modules were synthesised commercially with a BsaI restriction enzyme site close to each end of the sequences and separately inserted into a standard cloning vector to provide L0 vectors. One DNA designated as “LIR module” (SEQ ID NO: 91) included the LIR region from BeYDV and had an upstream BsaI-ligation site for cloning into a GoldenGate L1 acceptor vector and a downstream BsaI-ligation site for joining with a second module, designated the “Pr/UTR” module (SEQ ID NO: 92), see FIG. 31. In this construction, the Pr/UTR module included a CaMV e35S promoter joined to a 5′ UTR sequence from tobacco mosaic virus (TMV), including an ATG start codon, for efficient transcription and translation of a downstream protein coding region of interest if that was desired. The 3′ end of the Pr/UTR module was designed to be compatible with an RNA coding sequence, specifically a hpRNA coding sequence (FIG. 31). In turn, 3′ end of the RNA coding fragment was made compatible with 5′ end of a “T” module. The T module included a 3′ polyadenylation region/transcription terminator and the necessary GV replicative regions downstream of the terminator. This sequence (SEQ ID NO: 93) was synthesised with a 35S transcription terminator (35S Tm) followed by the SIR/Rep/RepA/LIR region from the BeYDV genome. The LIR module has the Fragment number EN38510, the Pr/UTR module (35S+TMV 5′UTR) has EN38509 and the T module (35S Tm SIR/Rep/RepA/LIR) has EN38511 for the GVc and EN38512 for the GVt vectors.

To provide for introduction of the GV sequence and expression of the RNA molecules in plant cells, the components were inserted into a binary vector, in this case pICH47742rc (FIG. 31). This resulted in the assembled components being inserted between the right and left T-DNA borders of the binary vector to provide for Agrobacterium-mediated introduction of the T-DNA including the GV vector sequences into plant cells, whereby the GV vector sequence becomes circularised and replicates and the incorporated RNA-encoding sequence is expressed to produce the RNA molecules.

Results with Replicating Vectors

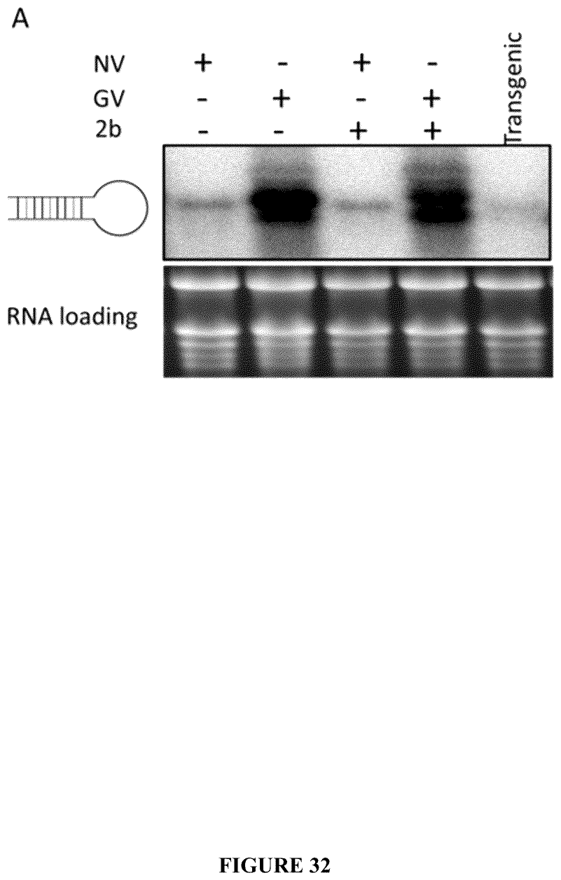

To assess the GV vector for expression and accumulation of RNA molecules, particularly modified hairpin RNA molecules, a DNA fragment encoding a G:U modified hairpin RNA (hpHa1[G:U]) was synthesized (SEQ ID NO: 94). The part of the fragment between its BsaI sites was inserted into the binary vector with the GV sequences by GoldenGate cloning using the BsaI/T4 ligation method as outlined above. This resulted in the insertion of the hpHa1[G:U] encoding region into the GV vector. Transformants containing the correct construct were confirmed by restriction digestion. The resultant construct was introduced into Agrobacterium tumefaciens strain GV3101 by electroporation with ampicillin as the selective agent. A non-replicative vector for expression of the same hpHa1[G:U] molecule from the 35S promoter was separately introduced into A. tumefaciens for comparison. The transformed Agrobacterium cells were infiltrated into N. benthamiana and N. tabacum leaves either with or without addition of the construct encoding the CMV 2b viral silencing suppressor protein. Infiltrated tissues were harvested from 1 day post-infiltration (dpi) to 7 dpi and total RNA extracted. The RNA samples were analysed by Northern blot using a radiolabelled probe to detect the full-length hairpin RNA. A representative blot for N. benthamiana at 4 dpi is shown in FIG. 32.

The blot showed that the hairpin RNA accumulated to much higher levels from the GV vector than from the non-replicative expression construct, even though both constructs used the same CaMV e35S promoter. The addition of the 2b silencing suppressor did not have a noticeable effect on the amount of full-length RNA accumulation. It was concluded that use of the GV vector to express hairpin RNAs was clearly beneficial in increasing the accumulation level.

Total RNA was also extracted from N. tabacum leaves from 1 dpi to 7 dpi and analysed for accumulation of the hpHa1[G:U] during the time course. Strong hpHa1[G:U] accumulation was observed from days 2 to 7, with the greatest level at day 3 after infiltration. Necrosis of the infiltrated zones in the leaves was observed from about day 5 onward. Addition of the 2b silencing suppressor protein reduced the amount of necrosis but did not eliminate it.

To quantitate how much hpRNA was accumulating in the agro-infiltrated Nicotiana tabacum leaves, samples were harvested at 2 dpi and 4 dpi and total RNA extracted. Northern blot hybridisation compared the band intensity from 10 μg samples of total RNA to that in a lane spiked with 50 ng of hpHa1 in vitro transcript loaded as size marker and a reference with a known quantity. Relative accumulation levels were calculated from the band intensity comparison. The results showed that the GV vector produced approximately 0.5-1 ng of hpRNA per microgram of total RNA at 2 dpi and 4.5-5.5 ng of hpRNA per microgram of total RNA at 4 dpi, while the non-GV vector produced approximately 0.4-0.8 ng of hpRNA per microgram of total RNA at 2 dpi and 0.6-1.1 ng of hpRNA/μg of total RNA at 4 dpi. It was concluded from this quantitation analysis that the GV vector produced substantially greater amounts of the hairpin RNA molecules.

In another experiment, the Agrobacterium cell density of the infiltration mixture was varied between an OD (600 nm) of 0.1 and 1.0. RNA was extracted from the agro-infiltrated N. tabacum leaves at 3 dpi and analysed by Northern blot. The results showed that the broad range of Agrobacterium cell densities for infiltration were all effective for hpRNA production, even at the lowest cell density tested. It was concluded that the use of Agrobacterium-mediated introduction of this type of GV-based vector was a broadly applicable method for producing the modified RNA molecules at high level in plant cells.

### Example 16: Design of Asymmetric RNA Molecules

### Design of Asymmetric Hairpin RNAs

The present inventors considered that DCL4, the main Dicer that processes long dsRNA molecules in plant cells, produces 21-mer sRNAs processively from a dsRNA portion of a hpRNA by measuring 21-basepair lengths along the dsRNA duplex, probably starting from the loop end of the duplex i.e. at the base of the loop. That is, the inventors conceived that DCL4 “counts” the length of 21 basepairs while ignoring, or at least tending to ignore, non-basepaired ribonucleotides that bulge out from the duplex. On this basis, the present inventors conceived of a way of increasing the production of 22-mer antisense RNAs in a eukaryotic cell by use of precursor hpRNA molecules with asymmetric bulges in the form of non-basepaired ribonucleotides in the antisense strand of the hpRNA, spaced at an average frequency of one non-basepaired ribonucleotide about every 22 ribonucleotides of the antisense sequence in the dsRNA portion of a hpRNA. The non-basepaired ribonucleotide could be engineered by single ribonucleotide insertions into the antisense sequence or preferably by deletion of single ribonucleotides from the sense sequence, leaving the antisense sequence unmodified, or a combination of such insertions and deletions. That is, when the dsRNA region is cleaved by DCL4 or other Dicers into double-stranded siRNAs having 21 nt from the sense strand, the corresponding antisense sequences would be 22 nt in length because of the presence of one non-basepaired ribonucleotide. Such siRNA molecules are designated herein as 21/22-mers, using the notation for the lengths of sense/antisense. Provided that the additional, non-basepaired antisense ribonucleotide was not close to either end of the siRNA, it would bulge out as a non-basepaired ribonucleotide in the siRNA duplex, without an opposing non-basepaired ribonucleotide in the sense sequence (e.g. FIGS. 53D and E). Since Dicers cleave a dsRNA region to produce siRNAs having non-basepaired 3′ di-ribonucleotide “overhangs”, the 21/22-mers would have, in a preferred form, 19 basepairs involving all of ribonucleotides 1-19 of the sense sequence. The 22 nt antisense RNA could then associate with AGO to form an effective RISC as well as promote secondary siRNAs against the target transcript. Clearly, the DNA sequences encoding the hpRNA could be readily engineered by single nucleotide insertions into the antisense sequence or by deletion of single nucleotides from the sense sequence, or a combination of such insertions and deletions, in order to efficiently produce the modified precursor hpRNAs by transcription.

It was also conceived that not every ribonucleotide in the resultant 21/22-mer siRNAs, aside from the extra non-basepaired ribonucleotide in the 22-mer antisense sequence and 3′ overhangs, needed to be basepaired in the 21/22-mer siRNAs. That is, one or more additional ribonucleotides in each of the 21-mer sense RNA sequence and 22-mer antisense RNA sequence might be non-basepaired, for example be present as a mismatched ribonucleotide pair. For example, 1, 2 or 3 additional ribonucleotides in the 22-mer antisense RNA sequence and 1, 2 or 3 ribonucleotides, respectively, in the 21-mer sense RNA sequence may be non-basepaired. Of these options, preferably at most only one additional ribonucleotide (making two in total) in the 22-mer antisense RNA sequence and one ribonucleotide in the 21-mer sense RNA sequence are non-basepaired in the 21/22-mer siRNA. Alternatively, or additionally, one or more ribonucleotides in each of the 21-mer sense RNA sequence and 22-mer antisense RNA sequence might be basepaired in a non-canonical basepair, preferably one or more G:U basepairs.

Furthermore, the present inventors conceived that production of 23-mer antisense RNAs could be increased by deletion of two ribonucleotides in every 23 ribonucleotides, on average, along the sense strand, or insertion of two ribonucleotides in every 21 nt of the antisense sequence, on average, to generate 23-mers, or any combination of insertions and deletions whereby sense sequences of 21 nt have a corresponding antisense sequence in the siRNA which is 23 nt in length i.e. 21/23-mers. The insertions or deletions could be of a di-ribonucleotide or of two single, spaced apart ribonucleotides. Additionally, the present inventors conceived that production of 24-mer antisense RNAs could be increased by deletion of three ribonucleotides in every 24 ribonucleotides, on average, in the sense strand, or insertion of three ribonucleotides on average in every antisense sequence of 21 nt to increase it to 24 nt, or any combination of insertions and deletions whereby every sense sequence of 21 nt has a corresponding antisense sequence in the siRNA which is 24 nt in length i.e. 21/24-mers. The insertions or deletions could be of a tri-ribonucleotide or of a di-ribonucleotide and a single ribonucleotide, spaced apart, or of three single, spaced apart ribonucleotides, or any combinations thereof.

The present inventors furthermore conceived of engineered precursor RNA molecules such as hpRNA molecules or ledRNA molecules which produce a combination of 21/22-mers and 21/23-mers, or 21/22-mers and 21/24-mers, or all three of 21/22-mers, 21/23-mers and 21/24-mers. Furthermore, each of these RNA molecules can also produce some 21/21-mer siRNAs, for example by having a part of the dsRNA region being unmodified as described above. DNA molecules encoding such precursor RNA molecules can be readily engineered.

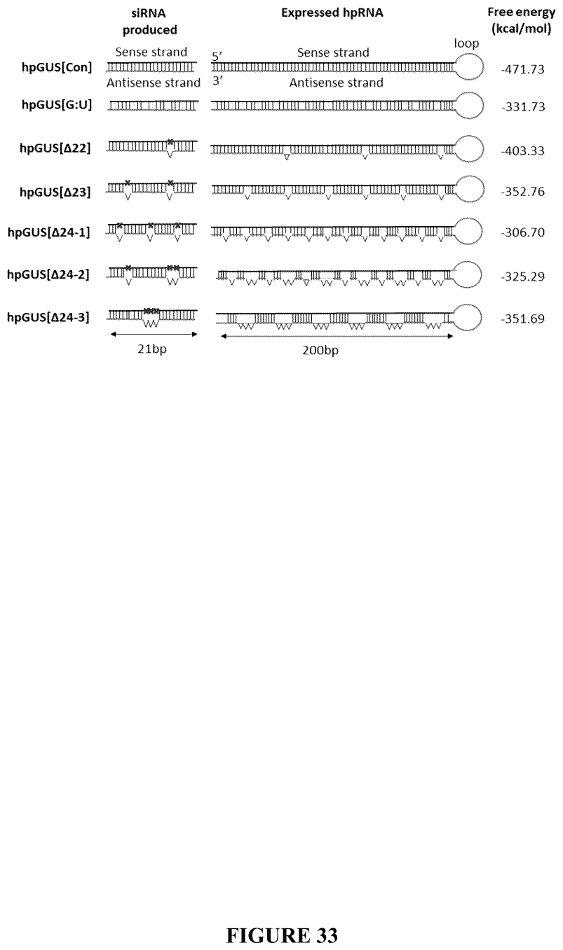

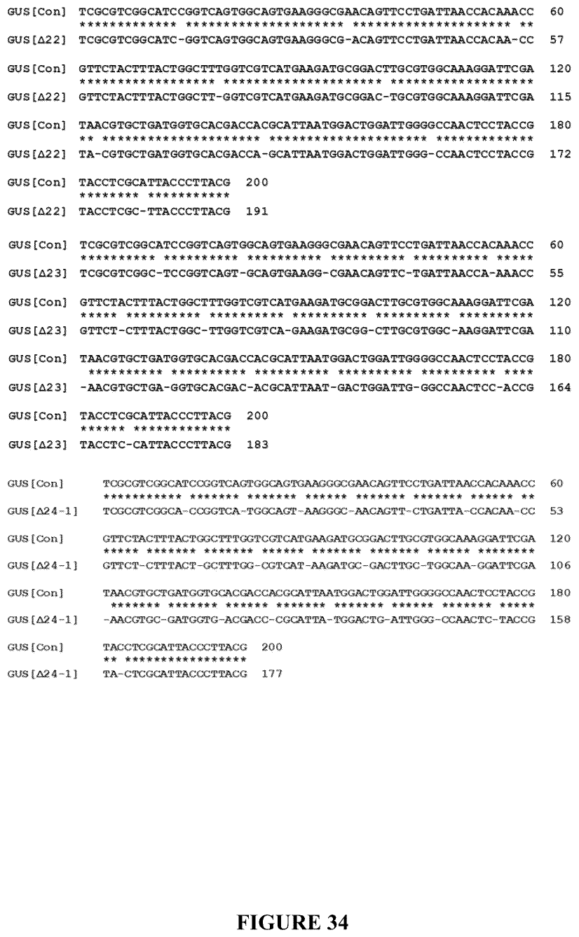

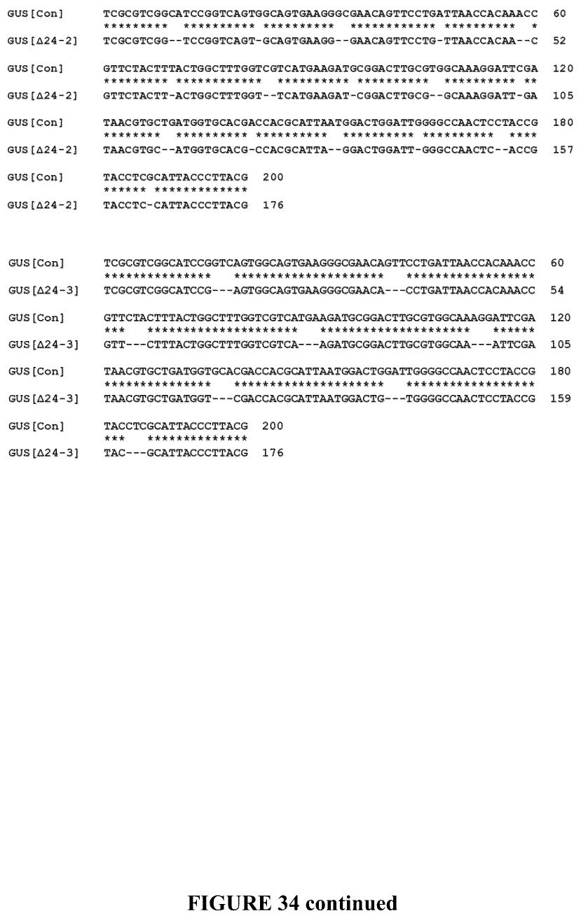

To test these designs, genetic constructs were made to express modified precursor hpRNAs with periodic ribonucleotide deletions in the sense strand to create one or more asymmetric bulges in the antisense strand of the resultant hpRNA molecules, where the bulges were created by one or more, specifically 1, 2 or 3, non-basepaired ribonucleotides in the antisense strand. In each case, all 19 of ribonucleotides 1-19 of the sense sequence in each siRNA produced by Dicer would basepair with ribonucleotides in the antisense sequence. Moreover, the antisense sequence was not modified relative to the antisense sequence of a conventional hpRNA used as a control, so the antisense sequence in each siRNA was fully complementary to a region of the target RNA molecule. These constructs were then tested to see whether the dominant antisense sRNA populations produced by processing of the modified hpRNA molecules by Dicers in the host cells could be changed from 21 nt to 22 nt, 23 nt or 24 nt in size, or combination of these larger sizes. The initial target transcript tested for each hpRNA design was from a gene encoding GUS as a reporter molecule. Five genetic constructs encoding asymmetric hpRNA versions were designed and made, aiming to produce 22 nt, 23 nt or 24 nt siRNAs from the antisense strand (FIG. 33). The construct encoding a hairpin RNA designated hpGUS[Δ22 nt], containing 1 non-basepaired antisense ribonucleotide on average every 22 ribonucleotides and aiming to produce 21 nt sense/22 nt antisense siRNAs (21/22-mers), was made by deleting about every 22nd nucleotide from the 200-nucleotide GUS sense sequence of the hairpin gene while leaving the antisense sequence unmodified and therefore still fully complementary to the target GUS transcript. That is, the hpGUS[Δ22] had multiple stretches of about 21 consecutive basepairs separated by a single non-basepaired ribonucleotide in the antisense strand, shown schematically in FIG. 33. The construct encoding a hairpin RNA designated hpGUS[423 nt], containing 2 non-basepaired antisense ribonucleotides about every 23 nt of antisense sequence and aiming to produce 21 nt sense/23 nt antisense siRNAs (21/23-mers), was made by deleting about every eleventh or twelfth nucleotide from the sense sequence of the hairpin gene. That is, the hpGUS[Δ23] had multiple stretches of 10 or 11 consecutive basepairs each separated by a single non-basepaired ribonucleotide in the antisense strand. For producing hairpin RNA molecules designated hpGUS[Δ24 nt] that aimed at yielding 21 nt sense/24 nt antisense siRNAs (21/24-mers), three versions were designed and made with different patterns of deletions, each with three nucleotides deleted from about every 24 nucleotides of sense sequence. The first version designated hpGUS[Δ24 nt-1] had 3 single unpaired ribonucleotides in each 24 nt antisense window, each unpaired ribonucleotide being spaced apart by 6 or 7 consecutive basepaired ribonucleotides. The second version designated hpGUS[Δ24 nt-2], had alternating 1 nt and 2 nt non-basepaired ribonucleotides that formed one- or two-nucleotide bulges in the antisense sequence of each siRNA, spaced apart by 10, 11 or 12 consecutive basepaired ribonucleotides. The third version designated hpGUS[Δ24 nt-3] had a single 3-ribonucleotide antisense bulge in every 24-nucleotide antisense sequence, so having multiple stretches of about 21 consecutive basepairs between the non-basepaired nucleotides (FIG. 33). Two genetic constructs were included as controls, one encoding a conventional hpRNA construct (hpGUS[Con]) without unpaired nucleotides in the dsRNA duplex, being fully canonically basepaired, and the second encoding a hpRNA that was also fully basepaired but including about 25% G:U basepairs in the duplex (hpGUS[G:U]) (Example 2 herein; Zhang et al, 2022). Both controls had the same 200 nucleotide antisense sequence complementary to the GUS transcript. FIG. 34 shows alignments of each modified sense sequence with the unmodified GUS sense sequence (SEQ ID NO: 2), showing the nucleotides that were deleted in the modified sense sequences.

These constructs were made as follows. The nucleotide sequences of the modified sense sequences used in the constructs are provided herein as SEQ ID NOs: 95-99. Each modified sense sequence for the GUS target gene was assembled by annealing overlapping forward and reverse oligonucleotide primers containing XhoI and KpnI sites, respectively. PCR extension of 3′ ends was performed using the high fidelity LongAmp Taq polymerase (New England Biolabs) and the synthesized fragments ligated into the pGEM-T Easy plasmid. The correct nucleotide sequences were confirmed. The DNA fragments having the modified GUS sense sequences were excised by digestion with XhoI and KpnI and ligated into the same sites of the hpGUS[WT] construct (Zhang et al., 2022), referred to herein as hpGUS[Con], to replace the wild-type sense sequence. The resulting binary plasmids were electroporated into E. coli strain DH5a and subsequently introduced into Agrobacterium tumefaciens strains GV3101 and LBA4404 for plant infiltration and transformation as described in Example 1. The resultant expression cassettes had the arrangement of elements as: 35S promoter-modified sense sequence-PDK intron-unmodified antisense sequence-Ocs 3′ polyadenylation region/transcription terminator. The nucleotide sequences encoding the hpRNA molecules and the DNA sequences corresponding to the stem-loop parts of the RNA molecules after removal of the intron by splicing are provided herein as SEQ ID NOs: 100-107.

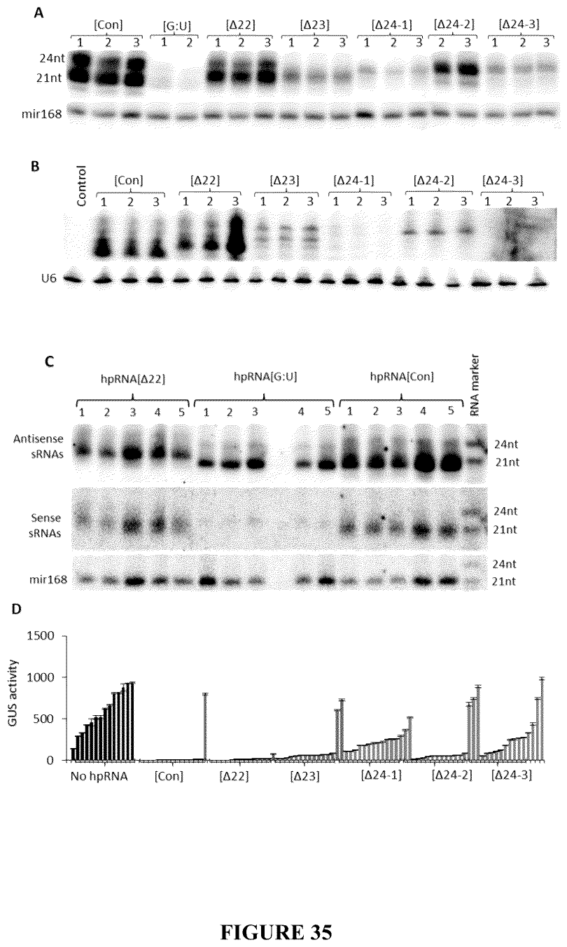

The asymmetric hpRNA constructs were first tested for their ability to produce altered antisense sRNA molecules by using Agrobacterium-mediated introduction experiments to transiently express the hpRNA constructs in non-transgenic N. benthamiana leaves, using the methods as described in Example 1. The Agrobacterium cells containing each construct (1) was mixed with Agrobacterium cells transformed with other constructs, as follows. In order to extend and increase the RNA production levels, a construct (2) to express the V2 silencing suppressor protein (WO2013/096992) was co-infiltrated with each hpRNA construct, as well as a construct (3) to express a GUS target transcript and another construct (4) encoding a GFP reporter to show successful gene introduction and expression. Mixtures of the four Agrobacterium strains each carrying a separate construct were prepared and infiltrated into the plant leaves with needleless syringes, as per Example 1. Control hpRNA constructs for (1) were hpGUS[Con] and hpGUS[G:U]. The transient assays were done in triplicate for each construct by infiltrating three leaves for each hpRNA construct. Three days after infiltration, total RNA was extracted from the infiltrated zones from two or three leaves for each construct and subjected to Northern blot analysis. The production of antisense sRNAs from the dsRNA regions of each of the hpGUS molecules was analysed using a radiolabelled probe corresponding to the sense strand of the hairpin molecules. RNA molecules of 21 or 24 nucleotides were used as size markers in the gels. A representative Northern blot is shown in FIG. 35, panel A. The hpGUS[Con] construct with the unmodified sense sequence yielded two intense siRNA bands which migrated at positions corresponding to 21 and 24 nucleotides in length, consistent with other reports for conventional hairpin RNAs. The hpGUS[G:U] construct also yielded two bands of the same sizes, but much less intense in this experiment, consistent with the results of Zhang et al. (2022). In contrast, all of the constructs encoding asymmetric hpGUS RNA molecules yielded antisense sRNAs with different sizes than the hpGUS[Con] construct, the sRNAs becoming progressively larger with the increasing number and size of the asymmetric bulges (FIG. 35, panel A). In particular, the hpGUS[Δ22] construct yielded one intense band for 22-mer antisense RNAs and a second, less intense band for 24-mers or 25-mers. The hpGUS[Δ23] and hpGUS[Δ24] constructs yielded fewer antisense sRNAs than the hpGUS[Con] and hpGUS[Δ22] constructs, but of sizes that appeared to include 23-mer and 24-mer sRNAs, respectively. It was considered that the greater number of non-paired nucleotides in these molecules may have reduced hpRNA processing through reduced stability of hpRNA molecules as determined by the free energy of folding (FIG. 35).

The transient expression experiment using N. benthamiana leaves was repeated except for omission in each infiltration of the construct expressing the GUS target gene (3 above). Northern blot hybridisation analysis of isolated RNA from the leaves detected essentially the same pattern of antisense sRNAs as before, from which it was concluded that the observed antisense sRNAs were produced from the expressed hairpin RNAs, not the target GUS transcript. It was also concluded that the change in size of the antisense sRNAs was not dependent on the presence of the target gene transcript.

The hpGUS constructs were then used to stably transform Nicotiana tabacum plants which already contained a target GUS transgene, designated PPGH24, in the Wi38 cultivar. The transgenic tobacco plants were produced as described in Example 1, using leaf tissues from PPGH24 plants (Wang et al., 1994). The doubly transformed TO plants were selected on tissue culture media containing 50 μg/ml kanamycin as the selective agent and confirmed to be transgenic by Northern blot hybridisation of RNA from the plants. Between 15-22 independently transformed plants were obtained for each construct. Total RNA was extracted from leaves of five independently transformed TO plants for each construct and analysed by Northern blot as before. The antisense sRNAs from the dsRNA regions of each of the hpGUS molecules were detected by hybridisation using a radiolabelled probe corresponding to the sense strand of the hpGUS[Con] molecule. A representative blot is shown in FIG. 35, panel B.

In another Northern blot, RNA samples from each of five independently transformed plants having the hpGUS[Δ22], hpGUS[G:U] and hpGUS[Con] constructs were prepared and analysed by Northern blot. The membrane was first hybridised with a radiolabelled probe corresponding to the sense strand to detect the antisense sRNAs (FIG. 35, panel C). The membrane was then stripped to remove bound sense probe and re-hybridised with an antisense probe to detect sense sRNAs. The results are shown in FIG. 35, panel C. It was observed that, as in the transient expression experiment (panel B), each of the hpGUS constructs having modified sense sequences with deleted nucleotides produced antisense sRNAs that were predominantly greater than 21 nucleotides in length. The hpGUS[Δ22] construct yielded predominantly 22-nucleotide antisense sRNAs, with some 23-mers and possibly some 25-mers. The hpGUS[A23] construct yielded antisense RNAs that appeared to be 23 nucleotides in length as well as a band for larger RNAs, probably 26-mers, and a fainter band at 22 nucleotides in length. The hpGUS[Δ24-1] and hpGUS[A24-2] constructs yielded an antisense band at 24 nucleotides in length, although it was not intense for the hpGUS[Δ24-1] construct. These results from stably transformed plants therefore confirmed the observations from the transient expression experiments in N. benthamiana, for production of altered lengths of antisense sRNAs.

The Northern blot probed with the antisense probe showed that the sense sRNAs produced from the hpGUS[Δ22] construct were the same size as from the hpGUS[Con] and hpGUS[G:U] constructs, at 21 nucleotides in length (FIG. 35, panel C). It was concluded from these N. benthamiana and tobacco experiments that the Δ22 modification to the hpGUS RNA resulted in processing to produce predominantly 22-mer antisense sRNAs with 21-mer sense RNAs (21/22-mers), and that these modified duplexes were predominantly processed (cleaved) by DCL4. This was confirmed by deep sequencing of small RNAs as described below. These experiments also provided confirmatory evidence that the hypothesis that initiated the experiments was correct, namely that DCL4 “counts” the length of 21 basepairs along the duplex for cleaving the two RNA strands, while tending to ignore non-basepaired ribonucleotides that bulge out from the duplex.

The doubly transformed tobacco plants were also assayed for GUS activity using the fluorometric 4-methylumbelliferyl-β-D-glucuronide (MUG) substrate as described in Example 1 in order to examine the efficiency of GUS silencing by the different hpRNA constructs. About 14 independent plants were assayed for each construct. The results are presented in FIG. 35, panel D. The plants transformed with the hpGUS[Δ22] constructs showed strong GUS silencing in all 14 (100%) of the doubly transformed plants, whereas the 13/14 of the plants transformed with the control hpGUS[Con] construct showed strong GUS silencing. The average reduction in GUS activity was 96% or 90%, respectively, across the independent transgenic plants for these constructs. The hpGUS[Δ23] plants had an average reduction in GUS activity of 75%, while the hpGUS[Δ24-1], hpGUS[Δ24-2] and hpGUS[Δ24-3] plants showed an average GUS reduction of 58%, 65% and 43% respectively (FIG. 35, panel D). The degree of GUS silencing appeared to correlate roughly with the amount of antisense sRNAs produced by the different hpGUS constructs (FIG. 35).

Several conclusions were evident from this experiment. Firstly, the 422 design showed increased gene silencing compared to the corresponding, conventional hairpin design, i.e. was more efficacious, and secondly, all five of the asymmetric hpRNA molecules were effective in reducing target gene activity. It was also concluded that at least one Dicer in the plant cells, probably DCL4, was efficiently cleaving the modified dsRNA duplexes, especially with the 422 variation, despite the introduction of non-basepaired bulges into the duplexes.

### Example 17: Asymmetric Hairpin RNAs Induce Abundant siRNA Production Downstream of the Target Sequence

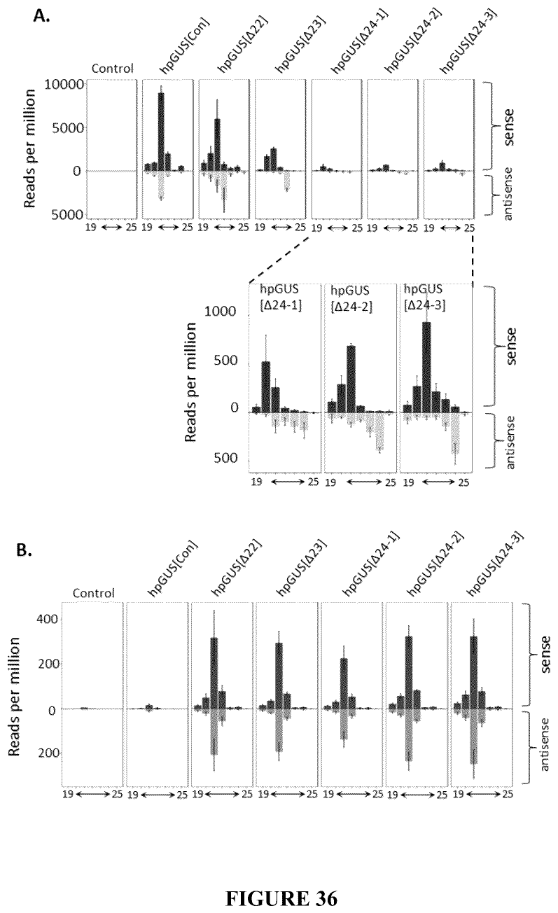

To further analyse the sRNA profiles produced from the hpGUS RNA molecules or induced from them (i.e. secondary sRNAs), total RNA samples were prepared from three independent transgenic plants for each of the hpGUS constructs and the sRNA populations in those samples were analysed by deep sequencing. This was done to determine the frequency of both sense and antisense sRNAs in the target region of the GUS gene and upstream (5′) and especially downstream (3′) of the target region. The sequences of the sRNAs were mapped to the GUS transcript sequence of 1,812 nucleotides (SEQ ID NO: 1), including the 200 nt hpGUS target region at nucleotides 804-1004. Reads were sorted by size for each of the classes having 19, 20, 21, 22, 23, 24 and 25 ribonucleotides in length, and for strand polarity i.e. sense or antisense sRNAs. The results are presented in FIG. 36 for sRNAs in the target region (panel A) or downstream of the target region (panel B). Consistent with the Northern blot analysis described above, the size profile of sense sRNAs from the target region of the hpGUS molecules was similar for all of the constructs, including for the conventional hairpin construct hpGUS[Con], with the 21 nt class being the most frequent (Panel A). The 22 nt class was second most frequent in the hpGUS[Con] plants, and the 20 nt class in the hpGUS[Δ22] and hpGUS[Δ23] plants. This observation suggested that the Dicer in the plants having the asymmetric hpGUSΔ22 and Δ23 constructs sometimes cleaved the RNA duplex after 20 basepairs, to produce a 20 nt sense/21 nt antisense siRNA (20/21-mer) in the case of hpGUS[Δ22] and a 20 nt sense/22 nt antisense siRNA (20/22-mer) in the case of hpGUS[Δ23]. In total, the 19-25 nt size class of sense sRNAs in the target region was more abundant in the plants transformed with the hpGUS[Con] construct but decreased in frequency in the plants transformed with the hpGUS[Δ22] construct and further decreased in the plants having the asymmetric hpGUS constructs with the Δ23 or Δ24 modifications. It was concluded that the duplexes having the non-paired nucleotides as bulges were less susceptible to cleavage by Dicer, with more non-paired nucleotides decreasing the sRNA production even more.

In contrast to the results for the sense sRNAs, the predominant size class of antisense sRNAs from the target region of the hpGUS molecules was dramatically altered, being dependent on the number and size of asymmetric bulges in the duplex i.e the number of non-basepaired ribonucleotides in the antisense strand. Plants having the hpGUS[Δ22] construct displayed a size change for the predominant antisense sRNA class from 21 to 22 nt, while the plants having the hpGUS[Δ23] construct showed a size shift from 21 to 23 nt (FIG. 36, panel A). Plants transformed with each of the hpGUS[Δ24] constructs gave a predominant antisense sRNA size class of 24 nt, although at relatively low abundance. Thus, all of the asymmetric hpGUS constructs generated sense and antisense sRNAs from the dsRNA portions of the introduced RNA molecules in the predominant size classes according to the hypothesis outlined above.

The deep sequencing analysis also provided data on whether sRNAs from the asymmetric hpGUS RNA molecules, especially the hpGUS[Δ22] molecule, could initiate the production of secondary siRNAs that mapped downstream of the target region. Such sRNAs are thought to arise when an antisense sRNA in the target region in the form of a RISC complex is effective in cleaving the target transcript and the RDR6/SGS3 system is activated to produce a complementary RNA strand 3′ of the target region. The resultant duplex RNA is then cleaved by Dicer, primarily DCL4, to produce secondary siRNAs, mapping downstream of the target region. The deep sequencing data were therefore examined for the frequency of sense and antisense sRNAs across the whole region of the GUS transcript downstream of the target region, for each of the hpGUS constructs, to determine the frequency and size classes of secondary sRNA production. The results are shown in FIG. 36, panel B. The results showed a dramatic difference between the hpGUS[Con] plants, which showed relatively few secondary sRNAs downstream of the target region, compared with each of the plants containing the asymmetric hairpin constructs where the secondary sRNAs were much more abundant. For example, the hpGUS[Δ22] plants showed a high frequency of 21 nt sense and antisense sRNAs across a region of about 800 nucleotides downstream of the hpGUS target region. Of note, all of the hpGUS[Δ23] and hpGUS[Δ24] plants also showed a greatly increased frequency of 21 nt sRNAs in this downstream region. Thus, all of the asymmetric hpRNA constructs induced production of considerable numbers of secondary sRNAs from the GUS target transcript. It was concluded that the predominance of the 21 nt size class of the downstream sRNAs for both sense and antisense sRNAs indicated that these sRNAs were produced from a fully basepaired duplex, without non-basepaired bulges, probably mostly by DCL4.

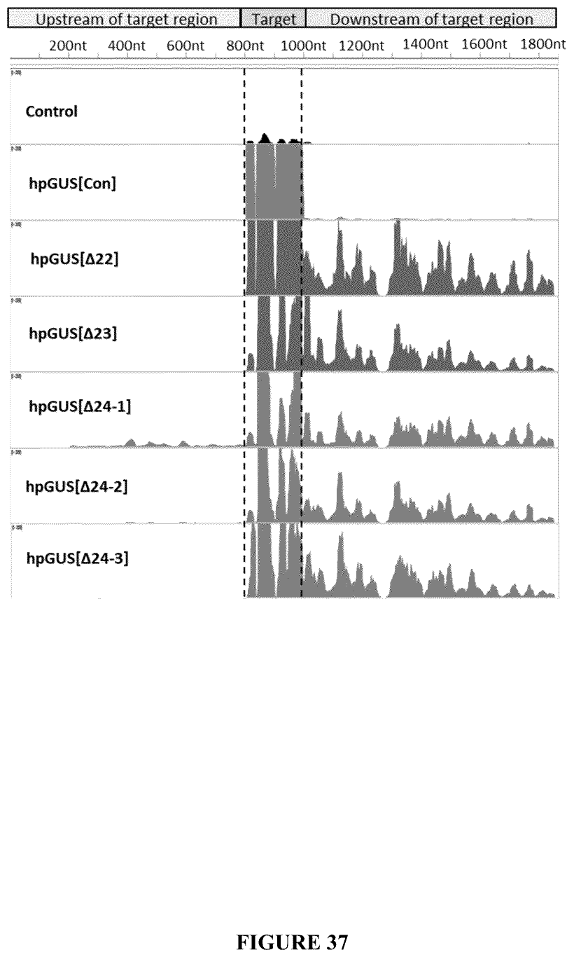

The sequences of the secondary siRNAs were mapped for their positions along the GUS target transcript, including both the upstream and downstream regions of the GUS target transcript. The frequency plots are shown in FIG. 37. This analysis showed that the secondary sRNAs were produced in the plants having the asymmetric hpGUS constructs in a variable, lumpy manner i.e. not at an even frequency across the entire downstream region. It was also noted that the hpGUS[Δ24-1] construct induced secondary siRNAs upstream as well as downstream of the target region, in contrast to the other asymmetric hpGUS constructs. It was concluded that there was some variability in the frequency of secondary sRNA production due to local sequence contexts. It was also thought that such a phenomenon would be true not just for the GUS target transcript but more generally for all RNA targets.

### Example 18: Asymmetric Hairpin RNAs Induce Effective Silencing Against Endogenous Genes

In order to test whether asymmetric hpRNA molecules could silence endogenous genes and induce secondary sRNA production from the transcripts of those endogenous genes, as for the GUS target gene as described above, two sets of genetic constructs were made that targeted endogenous genes in Arabidopsis thaliana. These were the genes Ethylene Insensitive 2 (EIN2) and Chalcone Synthase (CHS) (Zhang et al., 2022). The nucleotide sequences of the cDNAs for these two genes are provided as SEQ ID NO: 108 and SEQ ID NO: 109, respectively. Reduction in activity of EIN2 can be observed by increased hypocotyl length of seedlings grown in vitro in the dark on a medium containing 1-aminocyclopropane-1-carboxylic acid (ACC). Reduction of activity of CHS can be observed through a reduction of the brown pigmentation in seed coats (Zhang et al., 2022).

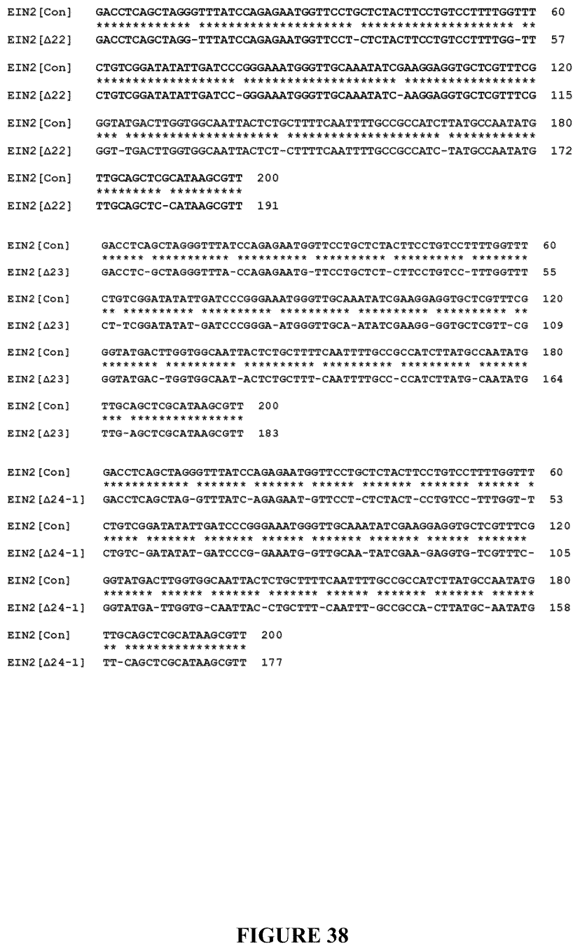

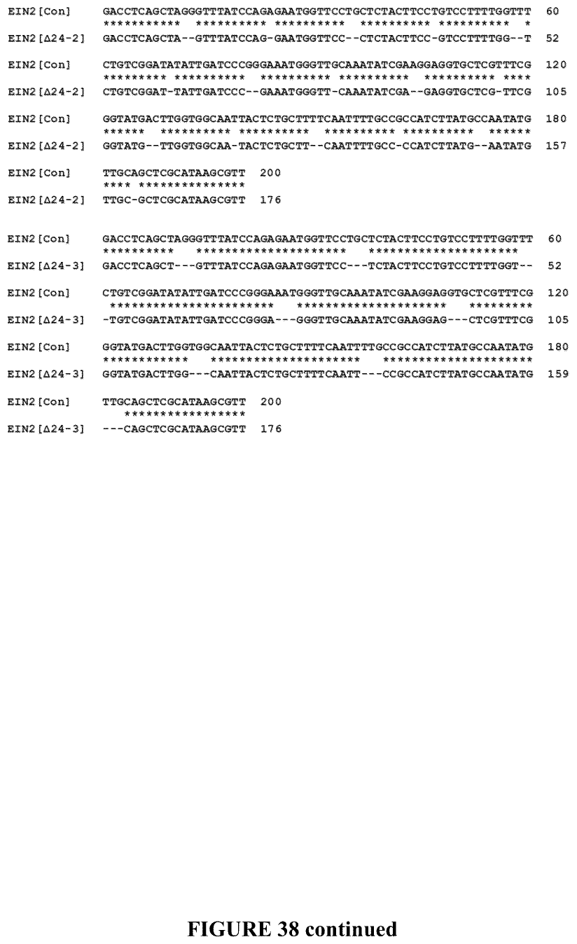

The two sets of genetic constructs were made by analogous methods to the hpGUS constructs described above, using each of the Δ22, Δ23, Δ24-1, Δ24-2 and Δ24-3 designs in both sets, with one set targeting the EIN2 transcript and the other the CHS transcript. In each case, a target region of 200 nt was selected in the target transcript, selecting a region toward 5′ end of the transcript: SEQ ID NO: 110 and SEQ ID NO: 116. These constructs were designated hpEIN2[Δ22], hpEIN2[Δ23] etc, in analogous fashion to the hpGUS constructs. The control constructs encoding the conventional hairpin RNAs were designated hpEIN2[Con] and hpCHS[Con]. Alignments of the wild-type sense sequences used in the control constructs and the corresponding modified sense sequences in the asymmetric hpEIN2 constructs are provided as FIG. 38 herein. The nucleotide sequences of the modified sense sequences used in the constructs for the hpEIN2 constructs are provided herein as SEQ ID NOs: 111-115.

Each of the constructs was used to transform A. thaliana plants of the Columbia ecotype using the floral dip method (Clough and Bent, 1998). At least 20 independent T1 transgenic plants were obtained for each of the twelve constructs. For each construct, T2 plants that were homozygous for the transgene were obtained from about 20 independently transformed T1 plants. Seed (T3 seed) were harvested from each the T2 plants and maintained as separate, homozygous transgenic lines.

### Reduction in Activity of the EIN2 Gene

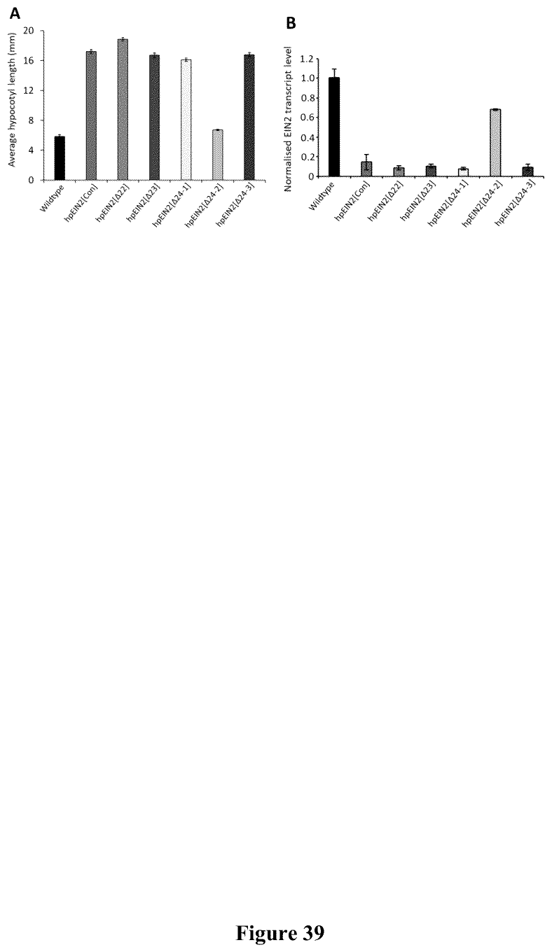

To assess EIN2 gene activity, at least 10 seed from each hpEIN2 transgenic line were plated on tissue culture medium with ACC and the selective agent kanamycin as described in Example 1. The plates were incubated for four days in the dark after which hypocotyl lengths were measured for at least 10 seedlings for each transgenic line. The average hypocotyl lengths from each of at least 5 independent transgenic lines are plotted graphically in FIG. 39, panel A. The asymmetric hairpin constructs hpEIN2[Δ22], hpEIN2[Δ23], hpEIN2[Δ24-1] and hpEIN2[Δ24-3], as well as the control construct hpEIN2[Con], all produced multiple transgenic lines which exhibited extended hypocotyl lengths compared to the wild-type A. thaliana Columbia plants, indicating strong reduction in EIN2 activity. However, the hpEIN2[Δ24-2] transgenic seedlings showed only a slight increase in average hypocotyl length compared to the wild-type control. A comparison of the five most affected hpEIN2[Δ22] lines demonstrated a 10% increase in hypocotyl length (p<0.01; Two-tailed Student Ttest) compared to the most affected hpEIN2[Con] lines (FIG. 39, panel A), thereby demonstrating increased endogenous gene silencing relative to the corresponding conventional hairpin RNA construct.

Next, the level of the EIN2 mRNA in the transgenic plants was measured by quantitative RT-PCR, as follows. Total RNA was extracted from the seedlings. To remove any DNA from the RNA preparations, 1 μg of RNA from each extracted sample was treated with DNase (RQ1 DNase, Promega Corp., Cat. #M6101) for 30 min followed by cDNA synthesis using oligo (dT) primer and Superscript III (ThermoFisher, Cat. #18080051). Each cDNA reaction was diluted with 180 μl of RNAse free water and 3 μl of diluted cDNA used per qRT-PCR reaction. Reactions were carried out with a Corbett 2000 Rotor-Gene real-time PCR machine (Qiagen) using FAST™ SYBR™ Green Master Mix (ThermoFisher, Cat. #4385612), 0.8 μM forward primer and 0.8 μM reverse primer. qRT-PCR was performed using two technical replicates for each sample, each in a 20 μl reaction. The EIN2 transcript level was normalized to the constitutive gene FDH using the 2-ΔΔCt method.

The results of the qRT-PCR analyses are shown in FIG. 39, panel B. The average level of EIN2 transcript was reduced by 85-94% by all of the hpEIN2 constructs tested except for hpEIN2[Δ24-2] which showed less of a reduction. It was observed that the asymmetric hpEIN2[Δ22], hpEIN2[Δ23], hpEIN2[Δ24-1] and hpEIN2[Δ24-3] constructs were at least as effective, in some cases more effective, at silencing the endogenous target gene than the conventional hpEIN2[Con] construct.

Analysis of sRNAs Induced by the Asymmetric Hairpin Constructs

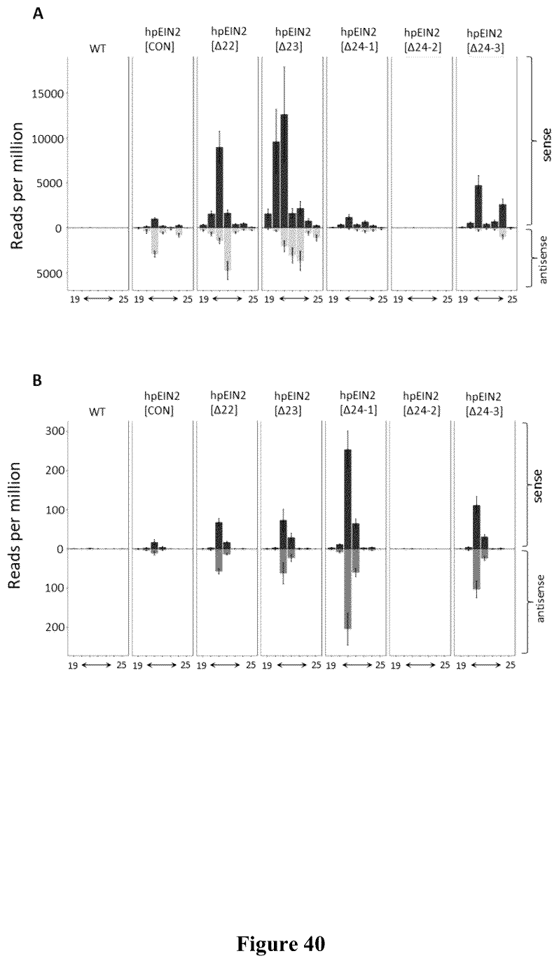

To analyse the size profiles of sRNAs produced from the hpEIN2 RNA molecules and induced secondary sRNAs that mapped to the EIN2 target transcript, total RNA samples were prepared from independent transgenic plants for each of the hpEIN2 constructs and the sRNAs of 19-25 nucleotides within each sample analysed by deep sequencing. This provided the frequency of both sense and antisense sRNAs in the target region of the EIN2 gene and downstream (3′) of the target region. The sequences of the sRNAs were mapped to the full-length EIN2 transcript sequence of 4,851 nucleotides, including the 200 nt hpEIN2 target region at nucleotide positions 654-853. Reads were sorted by size for each of the classes having 19, 20, 21, 22, 23, 24 and 25 nucleotides in length, and for strand polarity i.e. sense or antisense sRNAs. The results are presented in FIG. 40 for sRNAs in the target region (panel A) or downstream of the target region (panel B). In similar fashion to the results for the hpGUS sRNAs described above, the size profile of sense sRNAs from the target region of the hpEIN2 molecules was similar in plants for all of the constructs, including for the conventional hairpin construct hpEIN2[Con], with the 21 nt class being the most frequent (Panel A). In total, the 19-25 nt size class of sense sRNAs in the target region were much more abundant in the plants transformed with the hpEIN2[Δ22], hEIN2[Δ23] and hpEIN2[Δ24-3] constructs than the hpEIN2[Con] construct, but very low in abundance in the hpEIN2[Δ24-2] plants. It was concluded that, at least in this experiment, the asymmetric hpEIN2 duplexes having the non-paired nucleotides as bulges were more likely to be cleaved by Dicer to produce sRNAs, or those sRNAs were more protected from degradation by association with AGO, or both.

In contrast to the results for the sense sRNAs, as for the observations with the GUS hairpins, the predominant size class of antisense sRNAs from the target region of the hpEIN2 molecules was dramatically altered, being dependent on the number and size of asymmetric bulges in the RNA duplex. Plants having the hpEIN2[Δ22] construct displayed a size change for the predominant antisense sRNA class from 21 to 22 nt, while the plants having the hpEIN2[Δ23] construct showed a size shift from 21 to 23 nt, although 22 nt sRNAs were also abundant (FIG. 40, panel A). Plants transformed with the hpEIN2[A24-3] construct gave a predominant antisense sRNA size class of 24 nt. Thus, these three asymmetric hpEIN2 constructs generated sense and antisense sRNAs from the dsRNA portions of the introduced RNA molecules in the predominant size classes according to the hypothesis outlined above, as for the hpGUS constructs. That is, the Dicer(s) processing the asymmetric RNA duplexes having non-basepaired bulges tended to ignore the bulges and cleave after 21 basepairs.

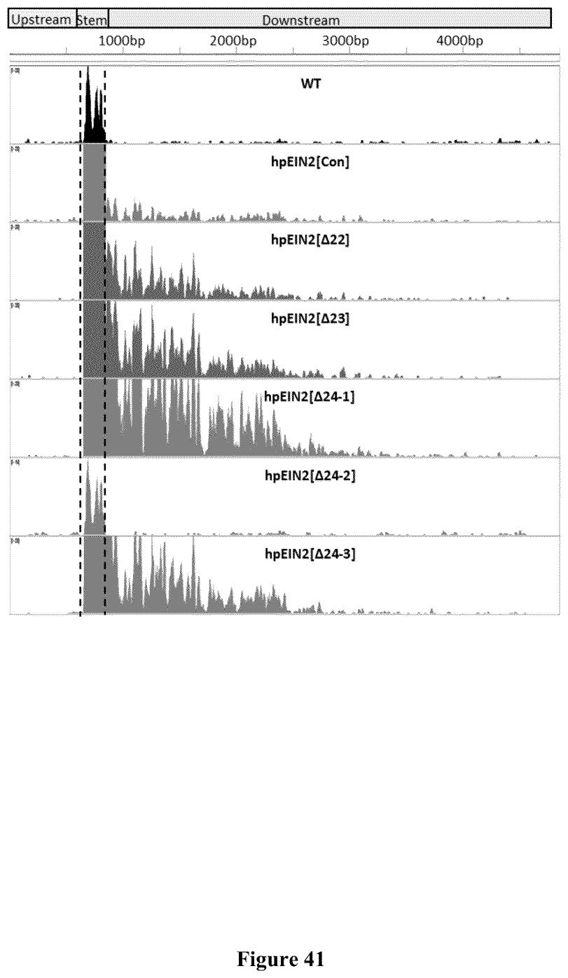

The deep sequencing analysis also provided data on whether sRNAs from the asymmetric hpEIN2 RNA molecules could initiate the production of secondary sRNAs that mapped downstream or upstream of the target region. The data were therefore examined for the frequency of sense and antisense sRNAs across the whole region of the EIN2 transcript downstream and upstream of the target region, for each of the hpEIN2 constructs, to determine the frequency and size classes of secondary sRNA production. The results are shown in FIG. 41. The results showed a dramatic difference between the hpEIN2[Con] plants, which showed relatively few secondary siRNAs downstream of the target region, compared to the each of the plants containing the asymmetric hairpin constructs where the secondary sRNAs were much more abundant. For example, the hpEIN2[Δ22] plants showed a high frequency of 21 nt sense and antisense sRNAs across a region of about 2,000 nucleotides downstream of the hpEIN2 target region. Of note, the hpEIN2[Δ23], EIN2[Δ24-1] and hpEIN2[Δ24-3] plants also showed a greatly increased frequency of 21 nt sRNAs in this downstream region. Thus, as for the asymmetric GUS constructs, the asymmetric hpEIN2 constructs induced production of considerable numbers of secondary sRNA from the EIN2 target transcript.

The 24 nt size class of sRNAs is known to induce RNA directed DNA methylation that can lead to transcriptional gene silencing (Erdmann and Picard, 2020). The extent of cytosine methylation in these plants was therefore examined by bisulfite sequencing. It was observed that plants containing the asymmetric hpEIN2 transgenes, including hpEIN2[A24 nt], surprisingly showed a lower level of DNA methylation at the target region of EIN2 than the hpEIN2[Con] plants, suggesting that the observed reduction in EIN2 gene activity had occurred through the post-transcriptional gene silencing pathway independent of DNA methylation. It was also concluded that the DNA constructs encoding the asymmetric RNA molecules were less prone to self-silencing i.e. less prone to being methylated, than the construct encoding the corresponding conventional (symmetrical) RNA molecule. This effect would also contribute to increased silencing efficiency.

### Reduction in Activity of the Endogenous CHS Gene

For the endogenous CHS target gene, a conventional hairpin construct hpCHS[Con] and an asymmetric hairpin construct hpCHS[Δ22] having the Δ22 modification in the sense sequence encoding the hairpin RNA were tested in stably transformed A. thaliana plants. Homozygous T2 plants were obtained for multiple independent transgenic lines, and mature seed was harvested from these. Seed from 19 randomly selected homozygous T2 lines transformed with the hpCHS[Δ22] construct exhibited greater loss of seed coat pigmentation than the hpCHS[Con] lines, with some of the hpCHS[Δ22] lines showing strong bleaching of the seed coat. This indicated a high degree of CHS gene silencing.

It was concluded from the EIN2 and CHS experiments that the asymmetric hairpin constructs were capable of efficient silencing of endogenous genes in plants. These results also demonstrated that the asymmetric hpRNA molecules, particularly with the A22 modification, produced greater gene down-regulation and stronger phenotypic changes than the corresponding conventional (symmetric) hpRNA RNA molecules.

### Example 19: Asymmetric Hairpin RNAs Enhance Plant Resistance to Viral Pathogens

In order to test whether asymmetric hpRNA molecules could reduce the activity of infecting viral genes and induce secondary sRNA production from the transcripts of those viral genes, thereby providing resistance against the viruses, a chimeric genetic construct was made that targeted both a gene from Cucumber Mosaic Virus (CMV) and a region from the Potato Virus Y (PVY) genome. PVY has a single stranded, positive sense RNA genome. Both of these viruses can infect solanaceous plants, for example CMV can infect Nicotiana tabacum and PVY can infect N. benthamiana, producing viral RNAs after inoculation with a viral dose and thereby yielding distinctive symptoms that can be scored visibly. Rather than targeting these with separate hpRNAs, a 300-nucleotide sequence from CMV was joined to a 300-nucleotide sequence from PVY in a single hairpin designated hpCMV/PVY[Δ22] for the asymmetric hairpin construct, or hpCMV/PVY[Con] for the corresponding conventional (symmetric) hairpin construct used as a comparator. The unmodified 300-nucleotide sense sequence from the CMV 2b gene (Accession No. LC376026) joined to the 300-nucleotide sequence from PVY (nucleotides 6217-6516 of Accession No. NC_001616) is presented herein as a chimeric sequence SEQ ID NO: 117.

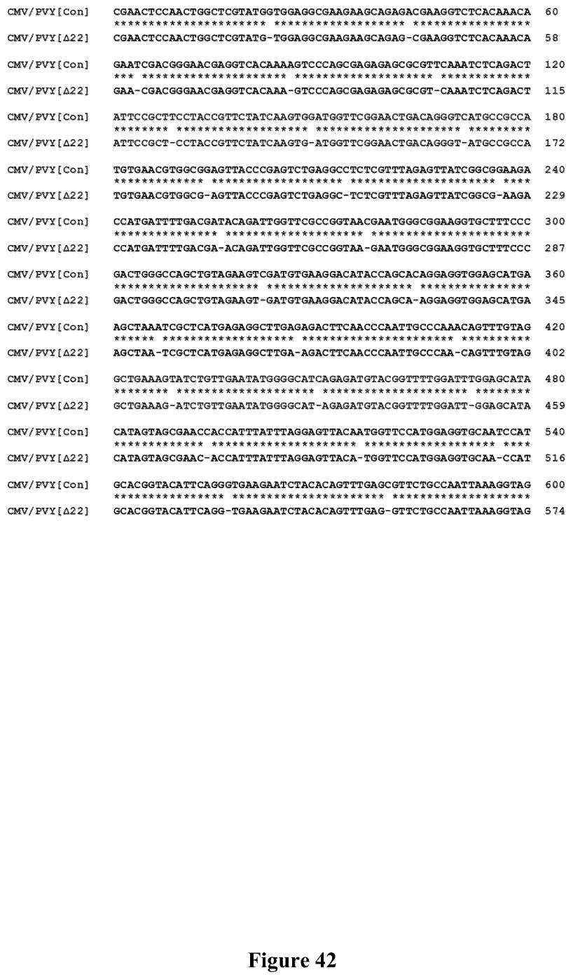

To make the modified sense sequence for the construct encoding hpCMV/PVY[Δ22], 26 nucleotides were deleted from SEQ ID NO: 117 using single nucleotide deletions spaced apart by 19, 20, 21 or 22 retained nucleotides in each case except for one spacing of 44 nucleotides, to make an average of a deletion about every 22 nucleotides. The nucleotide sequence of the modified chimeric sense sequence is provided as SEQ ID NO: 119, and the alignment of the modified sense sequence with the unmodified chimeric sequence is provided as FIG. 42, showing the positions of the 26 single nucleotide deletions. The construct encoding hpCMV/PVY[Δ22] for plant expression was made with the components in the order: CaMV 35S promoter-T7 RNA polymerase promoter-sense sequence-CAT1 intron-antisense sequence-polyadenylation region/transcription terminator; the sequence is provided herein as SEQ ID NO: 120. A T7 RNA Polymerase promoter with a GGG trinucleotide for efficient transcription initiation was included immediately upstream of the sense sequence to provide for in vitro transcription using T7 RNA Polymerase. The dual promoters in the one construct thereby allowed for expression in a cell free system by in vitro transcription as well as in the plant cells. The nucleotide sequence encoding the conventional hairpin RNA hpCMV/PVY[Con] is provided herein as SEQ ID NO: 118.

The asymmetric hpRNA construct hpCMV/PVY[Δ22] and the symmetric hpRNA construct hpCMV/PVY[Con] were each used to transform Nicotiana tabacum (cultivar Wi38) and Nicotiana benthamiana plants using the methods as described in Example 1. As negative controls, a pair of corresponding hpRNA constructs targeting the mGFP5 version of the gene encoding Green Fluorescent Protein, hpGFP[Con] and hpGFP[Δ22], were also used to transform N. tabacum and N. benthamiana plants. These control constructs had no sequence homology with the CMV and PVY sequences and therefore would not provide sequence-specific resistance against the viruses. About 50 confirmed, independent transgenic plants (TO) were obtained for each construct and 25 of them challenged with CMV to test for increased resistance to the virus. Ten wild-type untransformed plants at about the same stage of growth were also inoculated with the virus

The resultant primary (TO) transgenic N. tabacum plants along with wild-type, non-transgenic plants as controls were grown for about one month in soil in the greenhouse and then inoculated with a high dose of CMV. The inoculum was from an infected leaf of a stock plant, ground in 0.01 M sodium phosphate buffer pH 7.2 with some carborundum at 1 g/5 ml buffer. For inoculation, two fully expanded leaves per plant were sprinkled with carborundum and 100-200 μl of the inoculum mixture was applied with gentle rubbing to abrade the leaf surface and allow virus entry. Viral symptoms were scored once per week for three weeks and scored on a scale from 0 for no symptoms to 5 for severe symptoms, observed by mottled symptoms in systemic leaves. The data for the severity of viral infection is shown in Table 4 with the number of plants observed for each numerical value for symptom severity.

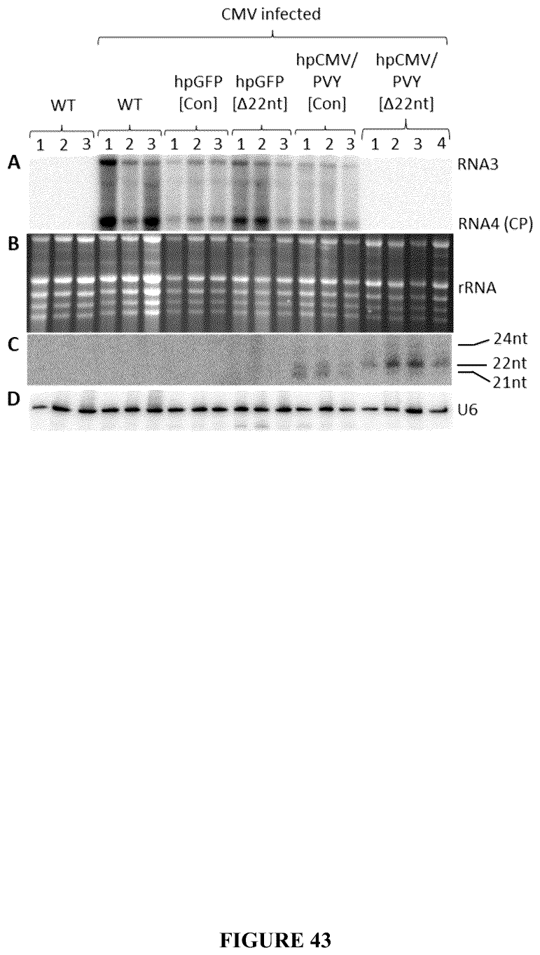

It was observed at 8 dpi that all of the wild-type (control), hpGFP[Con], hpGFP[Δ22] and hpCMV/PVY[Con] plants that had been inoculated with CMV displayed at least some symptoms of viral infection, with varying degrees of symptoms (Table 4). However, 32% (8/25) of the CMV-inoculated hpCMV/PVY[Δ22] plants showed no visible viral symptoms, and other plants showed only the mildest of symptoms (disease score of 1), indicating substantial resistance to virus infection. By 21 days post-inoculation, all of the transgenic plants displayed viral symptoms except for 20% of the hpCMV:PVY [Δ22] plants, which remained symptomless and were therefore considered immune. Northern blot analysis (FIG. 43, panel A) using a probe to detect viral RNAs in RNA extracted from upper, uninoculated leaves from the transgenic plants confirmed that the symptomless hpCMV/PVY[Δ22] plants lacked detectable replicating virus, while all of the other infected plants showed the presence of viral RNA. Northern blot analysis for sRNAs confirmed that the hpCMV/PVY[Δ22] plants immune to CMV infection all accumulated siRNAs with a dominant size class of 22 nt, in contrast to the hpCMV/PVY[Con] lines that accumulated a predominant 21 nt siRNAs as well as some 22 nt sRNAs (FIG. 43, panel C).

To confirm the results from the TO plants, T1 seed obtained from two independent transgenic plants for each construct were sown and the resultant T1 progeny plants obtained and tested by viral challenge. Twenty plants per line were used, with 2-5 individual plants not inoculated per line. This generation of plants was expected to be segregating for the transgenes, so plants containing the transgene were identified. About 15 T1 sibling plants for each of two lines transgenic for hpCMV/PVY[Con] or hpCMV/PVY[Δ22] were inoculated with a high dose of CMV, as before. After one week, the hpCMV/PVY[Δ22] populations showed a clear increase in resistance to the virus, with an average of 77% of plants across the two lines displaying no symptoms compared to an average of 20% of plants for the two hpCMV/PVY[Con] lines. After 3 weeks, more than 95% of the hpCMV/PVY[Con] plants displayed symptoms of viral infection while 77% of hpCMV/PVY[Δ22] plants remained symptomless. Northern blot analysis confirmed the absence of replicating CMV RNA in the symptomless hpCMV/PVY[Δ22] plants, showing that the Δ22 modification was effective in preventing viral replication and protecting the plants.

In similar fashion, viral resistance was assessed for the primary (TO) transgenic N. benthamiana plants containing the same set of hpRNA constructs. Plants were inoculated with PVY in the same way as for CMV and scored for viral symptoms such as vein clearing over a 3-week period. The hpCMV/PVY[Con] and hpCMV/PVY[Δ22] transgenic plants both displaying some resistance with 35% and 48%, respectively, remaining symptomless over the 3-week period. As for CMV, the hpCMV/PVY[Δ22] plants displayed less severe symptoms compared to the hpCMV/PVY[Con] plants. Viral RNA titres were consistent with the observed phenotypes. Taken together, these data demonstrate that increasing the production of 22 nt antisense sRNAs targeting viral pathogen RNA transcripts through the Δ22 modification enhanced plant resistance to virus infection compared to the corresponding unmodified RNA molecules. In these instances, the modified RNA molecules were expressed in the plants prior to viral infection, providing a level of an inbuilt protection.

### Example 20: RNA Constructs Targeting a Family of DDM1 Genes in Brassica napus

### Introduction

As described in previous examples herein, the present inventors successfully manipulated the RNAi pathways to produce increased numbers of 22, 23 and 24 nt antisense sRNAs directed against a target RNA transcript by changing the size and frequency of asymmetric bulges in the double-stranded regions of a precursor RNA molecule. The 22 nt antisense sRNAs then triggered production of secondary siRNAs from the target RNA transcript, thereby providing for amplification of the effect of a limited number of primary RNAi molecules and greatly increasing the suppressive function. The present inventors considered that the amplification effect occurred, after cleavage of the target RNA transcript by a 22-mer antisense sRNA incorporated into a RISC, through recruitment of SGS3 and RDR6 proteins to produce a double-stranded RNA molecule from the cleaved target RNA transcript, starting downstream of the targeted region. This occurs from 3′ end of the cleaved target RNA transcript in many cases but occurs in a region of about 2000 nucleotides downstream of the target region for longer target RNA transcripts such as EIN2 (FIG. 41). This double-stranded RNA is then further processed into multiple secondary siRNAs to provide the increased target silencing.

TABLE 4
Number of transgenic plants showing disease scores of 0
(no symptoms) to 5 (most symptomatic) at 8, 14 and 21
days post inoculation with a high dose of CMV, compared
to non-transgenic wild-type plants (WT). The identity
of each transgene is shown at the head of each column.

| | Number of plants showing disease score | | | | |
|---|---|---|---|---|---|
| Disease | | | | hpCMV/ | hpCMV/ |
| Score | WT | hpGFP[Con] | hpGFP[Δ22] | PVY[Con] | PVY[Δ22] |

8 days post inoculation

| | | | | | |
|---|---|---|---|---|---|
| 0 | 0/10 | 0/25 | 0/25 | 0/25 | 8/25 |
| 1 | 0/10 | 0/25 | 1/25 | 5/25 | 8/25 |
| 2 | 0/10 | 7/25 | 6/25 | 14/25 | 5/25 |
| 3 | 10/10 | 15/25 | 17/25 | 6/25 | 4/25 |
| 4 | 0/10 | 3/25 | 1/25 | 0/25 | 0/25 |
| 5 | 0/10 | 0/10 | 0/25 | 0/25 | 0/25 |

14 days post inoculation

| | | | | | |
|---|---|---|---|---|---|
| 0 | 0/10 | 0/25 | 0/25 | 0/25 | 4/25 |
| 1 | 0/10 | 0/25 | 0/25 | 0/25 | 5/25 |
| 2 | 0/10 | 1/25 | 1/25 | 0/25 | 0/25 |
| 3 | 0/10 | 8/25 | 3/25 | 6/25 | 9/25 |
| 4 | 8/10 | 15/25 | 21/25 | 17/25 | 7/25 |
| 5 | 2/10 | 1/25 | 0/25 | 2/25 | 0/25 |

21 days post inoculation

| | | | | | |
|---|---|---|---|---|---|
| 0 | 0/10 | 0/25 | 0/25 | 0/25 | 5/25 |
| 1 | 0/10 | 0/25 | 0/25 | 0/25 | 4/25 |
| 2 | 0/10 | 0/25 | 0/25 | 0/25 | 3/25 |
| 3 | 0/10 | 0/25 | 0/25 | 6/25 | 8/25 |
| 4 | 5/10 | 18/25 | 22/25 | 17/25 | 5/25 |
| 5 | 5/10 | 7/25 | 3/25 | 2/25 | 0/25 |

The inventors therefore considered that this modification would also increase the effectiveness of the asymmetric precursor RNA molecules when applied topically, to reduce target gene activity and provide for desired phenotypic changes. To test this, a series of modified RNA molecules including asymmetric RNA molecules were generated and tested against the DDM1 genes in Brassica napus, as a model for a target gene family. This small family of genes encodes methyltransferases which methylate cytosine bases in DNA (Zhang et al., 2018). Reduction of DDM1 gene expression has been shown to decrease DNA methylation and increase the number and position of recombinational cross-over events in A. thaliana (Melamed-Bessudo et al., 2012). Genetic constructs encoding precursor ledRNA molecules with either symmetric or asymmetric (Δ22) double-stranded regions, and conventional hairpin RNA molecules as a comparison, were prepared and transcribed in vitro to produce the RNA molecules, as follows. The precursor RNA molecules were then delivered exogenously i.e. topically, rather than being produced from a transgene in the plant cells.

Target Genes and Construction of ledRNA Molecules

B. napus is an allotetraploid species and has two DDM1 genes on each of the A and C subgenomes, on chromosomes A7, A9, C7 and C9, therefore having a total of four DDM1 genes in the gene family. These genes are designated BnaA07g37430D-1, BnaC07g16550D-1, BnaA09g52610D-1 and BnaC09g07810D-1. The nucleotide sequence of the cDNA corresponding to one of these genes, BnaA07g37430D-1, is provided herein as SEQ ID NO: 121 (NCBI Accession No. XR_001278527.2). Two regions of the cDNA nucleotide sequence were selected to design the hpRNA and ledRNA constructs targeted all four of the DDM1 genes present in B. napus, based on sequence conservation between the genes. These two regions corresponded to nucleotides 648-959 and nucleotides 2029-2218 of SEQ ID NO: 121. The nucleotide sequences of these two regions were greater than 90% identical across the cDNA sequences for the four DDM1 genes in B. napus.

A conventional hairpin RNA construct was designed and made using the 502-nucleotide chimeric sense sequence (SEQ ID NO: 122) and its complement as the antisense sequence, separated by a linking sequence comprising an intron which would be spliced out in vivo. To make the DNA construct encoding the hairpin RNA, designated hpDDM1[Con], the 502-nucleotide fragment was inserted in sense and antisense orientations in the pHellsgate 8 vector, resulting in an interrupted inverted repeat structure. The intervening sequence with the intron provided increased stability in the cloning process when introduced into E. coli. The order of elements in the construct was promoter-sense sequence-intervening sequence-antisense sequence-transcription terminator/polyadenylation region. The DNA construct included the PDK1 intron from the pHellsgate 8 vector separating the sense and antisense sequences, which after excision by splicing would leave a tight loop of 4 nucleotides. A T7 RNA polymerase promoter was also present immediately upstream of the RNA transcribed region to allow for in vitro transcription, in addition to the upstream CaMV 35S promoter for expression in the plant cells. The nucleotide sequence of the chimeric DNA encoding the hpDDM1[Con], without the 35S promoter, is provided as SEQ ID NO: 123.

A corresponding, second construct was also made encoding a hairpin RNA targeting the same 502-nucleotide region and having the same structure except that 97 cytosine nucleotides (C) of the sense sequence were replaced with thymidine nucleotides (T). When the chimeric DNA was transcribed and the G:U substituted hpDDM1 [G:U] RNA was self-annealed, this provided for 97/502=19.4% of the nucleotides in the dsRNA region being basepaired in a G:U basepair.

A third, chimeric DNA encoding a symmetric ledRNA, designated ledDDM1[Con], was made using the same sense and antisense sequences as the construct encoding hpDDM1[Con] and therefore targeting the same regions of the DDM1 genes of B. napus. The antisense sequence was split into two halves, so that the order of the sequence elements from 5′ to 3′ was promoter-antisense 1 sequence-loop 1-chimeric sense sequence-loop 2 sequence-antisense 2 sequence-transcription terminator/polyadenylation region. The nucleotide sequence of this chimeric DNA encoding ledDDM1[Con] is provided herein as SEQ ID NO: 124.

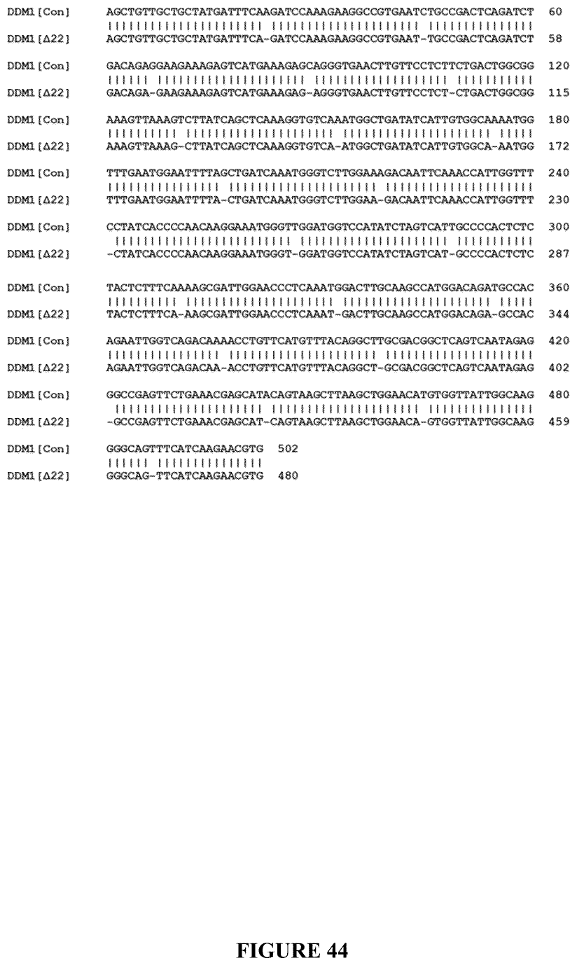

A fourth, chimeric DNA encoding a corresponding asymmetric ledRNA with the Δ22 modifications, designated ledDDM1[Δ22], was made using the same antisense sequences but with the chimeric sense sequence having 22 single-nucleotide deletions on average about every 22nd nucleotide. The nucleotide sequence of the chimeric sense sequence with the 22 nucleotide deletions is provided herein as SEQ ID NO: 125. As for ledDDM1[Con], the corresponding antisense sequence was split into two halves, so that the order of the sequence elements was promoter-antisense 1 sequence-loop 1 sequence-modified sense sequence-loop 2 sequence-antisense 2 sequence-transcription terminator/polyadenylation region. The nucleotide sequence of this chimeric DNA encoding ledDDM1[Δ22] is provided as SEQ ID NO: 126. An alignment of the modified sense sequence with the unmodified sense sequence, indicating the positions of the 22 single-nucleotide deletions, is shown in FIG. 44.

Reduction of DDM1 Gene Transcripts after Topical Application of the Precursor RNAs

For production of the RNAs by in vitro transcription, the DNA preparations were cleaved with the restriction enzyme HincII which cleaved immediately after the RNA coding region to provide termination of transcription. The DNAs were transcribed in vitro with T7 RNA polymerase. The RNAs were purified and then concentrated in an aqueous buffer solution. B. napus (canola) cotyledons from five-day-old seedlings grown aseptically on tissue culture medium were carefully excised and placed in a petri dish containing 2 ml MS liquid media, containing 2% (w/v) sucrose with 113 μg of RNA transcript or 100 μl of aqueous buffer solution as a control. The MS liquid medium used for the treatments contained Silwett-77, a surfactant (0.5 μl in 60 ml). The petri dishes were incubated on a shaker at room temperature with gentle shaking, so that the cotyledons soaked in the solution containing the ledRNA. Samples were harvested after 5 hr and 7 hr of soaking. In a parallel experiment, the upper surface of cotyledons was coated with either 10 μg of RNA transcript in buffer or the buffer solution without RNA and incubated on a wet tissue paper. Samples were collected 7 hr after RNA application and analysed.

Furthermore, in order to target the DDM1 endogenous transcripts in reproductive tissue of B. napus, unopened canola floral buds from plants just prior to flower opening were excised and then exposed to ledDDM1[Con] or ledDDM1[Δ22] RNA either in the presence or absence of non-transformed Agrobacterium tumefacians strain AGL1 cell suspension, i.e. living AGL1 cells. Aqueous buffer solution with or without the AGL1 cells served as respective controls. The AGL1 was grown in 10 ml of LB liquid media containing 25 mg/ml rifampicin for two days at 28° C. The cells were harvested by centrifugation at 3000 rpm for 5 minutes. The cell pellet was washed and the cells resuspended in 2 ml liquid MS media. Floral buds were incubated in a petri dish containing 2 ml of MS liquid media, including 0.5 μl of Silwett-77 in 50 ml of MS liquid media, with 62 μg of ledDDM1[Con] or ledDDM1[Δ22] RNA, with or without addition of 50 μl of AGL1 culture. As controls, 50 μl of buffer solution or 50 μl of buffer solution with 50 μl of AGL1 culture was used for treating floral buds in parallel fashion. Samples were incubated on a shaker with gentle shaking for 7 hr at room temperature. Three biological replicates were used for each of the treatments.

The treated and control cotyledons and floral buds were washed twice in sterile distilled water, the surface water removed using a tissue paper and the tissues flash frozen with liquid nitrogen. RNA was isolated from the treated and control tissues, treated with DNase to remove genomic DNA and the amount of DDM1 mRNA quantitated. First strand cDNA was synthesized using equal amounts of total RNA from ledRNA-treated samples and their respective controls. Expression of DDM1 was analysed using quantitative real-time PCR (qRT-PCR).

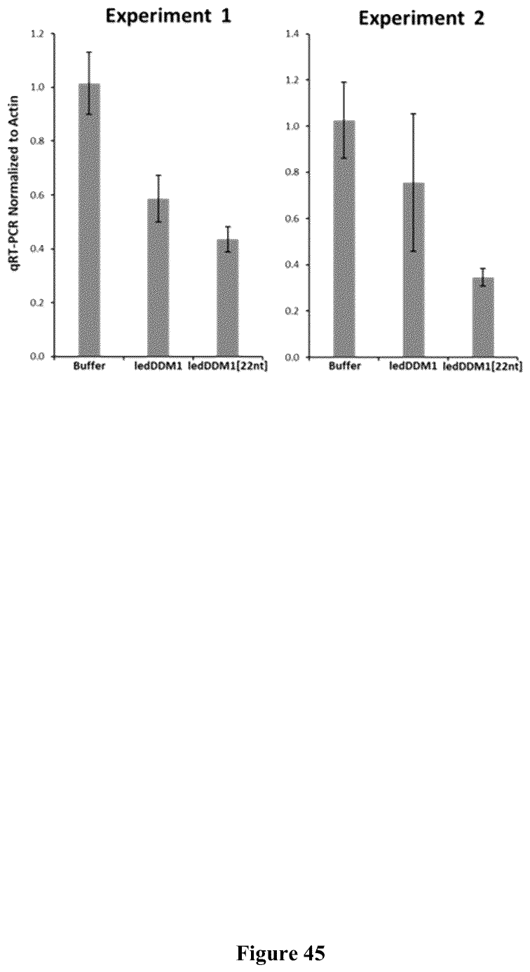

In the treated cotyledons that were soaked with the ledDDM1[Con] or ledDDM1[Δ22] RNA, DDM1 transcript abundance was decreased by 83-86% at 5 hr, which decreased further with a reduction of 91% at 7 hr compared to the controls. Similarly, a reduction of 78-85% in the DDM1 mRNA level compared to the control was observed in cotyledons that were coated with ledDDM1[Con] or ledDDM1[Δ22] RNA. No difference compared to the control was detected for DDM1 mRNA abundance in the floral buds that were treated with ledDDM1[Con] or ledDDM1 RNA in the absence of Agrobacterium cells. However, a reduction of approximately 60-75% in DDM1 transcript levels was observed in floral buds that were treated with ledDDM1[Con] or ledDDM1[Δ22] RNA in presence of Agrobacterium compared to its respective control. In two separate experiments, the ledDDM1[Δ22] had greater reduction of the target mRNA compared to the buffer control or the ledDDM1[Con] construct (FIG. 45). No significant difference in DDM1 transcript levels was detected when the control without Agrobacterium was compared with the control that had Agrobacterium, showing that the Agrobacterium cells themselves were not causing the decrease in DDM1 transcript. Taken together, these results indicated that the ledDDM1[Con] and ledDDM1[Δ22] RNAs were able to reduce endogenous DDM1 transcript levels in both cotyledons and floral buds, with greater effect of the latter, while living Agrobacterium cells appeared to facilitate the ledRNA entry into the floral buds. Such accessibility of the ledRNA molecules might also be achieved by physical means such as piercing the outer layers of the floral buds, centrifugation or vacuum infiltration, or a combination of such methods.

For another construct targeting the endogenous DDM1 genes, a construct was designed to express a ledRNA molecule having A to G substitutions in the sense sequence, providing for G:U basepairs in the double-stranded region of the RNA molecules designated ledDDM1[G:U]. This molecule had an unmodified antisense sequence and so the antisense sRNAs produced from the precursor ledDDM1[G:U] molecules would be fully complementary to the target mRNA, as before. Alternately, not all of the A nucleotides in the sense sequence were replaced with G's. In particular, where a run of 3, 4 or 5 contiguous A's occurred in the sense sequence, only 1 or 2 of the three A's, or only 2 or 3 of four A's, or only 2, 3 or 4 of 5 contiguous A's, were replaced with G. This provided for a more even distribution of G:U basepairs in the double-stranded RNA region. The longest stretch of contiguous canonical basepairing in the double-stranded region was 15 basepairs, and the second longest 13 contiguous basepairs.

A further ledRNA construct was designed where one or two basepairs in every block of 4, 5, 6 or 7 nucleotides in the sense sequence was modified with C to T or A to G substitutions. Where the wild-type sense sequence had a stretch of 8 or more nucleotides consisting of T's or G's, one or more nucleotides were substituted either in the sense strand to create a mismatched nucleotide within that block or a C to T or A to G substitution was made in the antisense strand, so as to avoid a double-stranded stretch of 8 or more contiguous canonical basepairs in the double-stranded region of the resultant ledRNA transcribed from the construct. The resultant ledRNA therefore would have G:U basepairs in the duplex region, on average at least one G:U basepair in every block of 7 basepairs in the duplex region, with the G ribonucleotides in the antisense strand or, preferably, in the sense strand.

A genetic construct was also designed which combined the ledRNA structure with the Δ22 modification, described above as ledDDM1[Δ22], additionally with the G:U basepairing modification as described above, targeting the same regions of the DDM1 transcripts. In vitro transcripts from the construct are applied to B. napus cotyledons and the reduction in the target DDM1 transcripts measured by qRT-PCR. The production of sRNA molecules from the target region and secondary, downstream siRNAs produced in the treated cotyledons and non-treated leaves are assayed by Northern blot.

### Example 21: Root Uptake and Systemic Silencing of the Endogenous Gene DDM1 Using Topically Applied RNA

Next, the present inventors tested the ability of three structurally different RNAi molecules to move from the root into the cotyledon after topical application, or for the resultant 22-mers to move in that direction. The constructs encoding hpDDM1[Con], ledDDM1[Con] and ledDDM1[Δ22] were as described above. In vitro transcribed RNA was dissolved in 50 mM HEPES buffer made in DEPC-treated water, pH 6.8, and quantitated by Qubit. B. napus seedlings were grown in tissue culture in the dark at 25° C. for 2 days to extend root and hypocotyl length. Six seedlings for each treatment were removed carefully from the medium and transferred into 0.5 ml Eppendorf tubes. Each tube contained 300 μl of treatment solution, with or without the RNA molecules, each solution containing 25 mM HEPES, half strength MS medium, 1% (w/v) sucrose and the detergent L-Silwet. The final RNA concentration was 100 μg/ml. Three seedlings were harvested after the 6 hr root soaking at room temperature. The other 3 seedlings were soaked for the 6 hr and then transferred into pots containing a seedling mix and grown under long day conditions (16 hr light/8 hr dark; 24° C. day/15° C. night) for 72 hr. When the seedlings were harvested, cotyledons were separated from hypocotyls and snap frozen in liquid N2. Roots were also separated from the hypocotyls and washed several times in water to remove any residual, external RNA from the soaking step before being frozen in liquid N2. At no point during the experiment did the cotyledons come in contact with the RNA solutions.

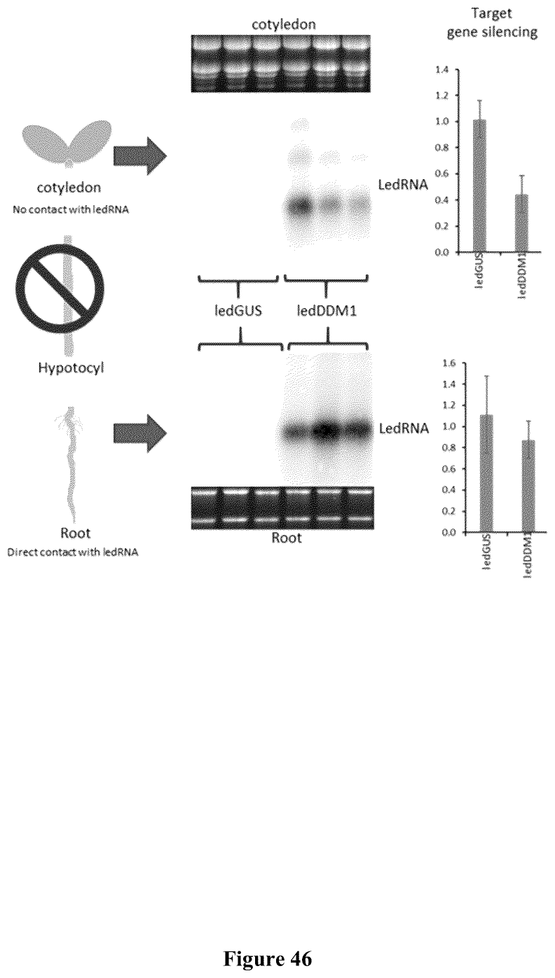

The uptake and movement of RNA molecules into the cotyledons was analysed through quantitative real-time PCR and northern blot analysis on total RNA extracted from the tissues. The effect on the target mRNA accumulation in the cotyledons was also measured by qRT-PCR. After the 6 hr exposure, the canola seedlings had taken up ledDDM1[Con] molecules which could be found within both the root and cotyledon (FIG. 46). The presence of ledDDM1[Con] molecules within the cotyledons demonstrated that the ledRNA molecules could move systemically through the plant.

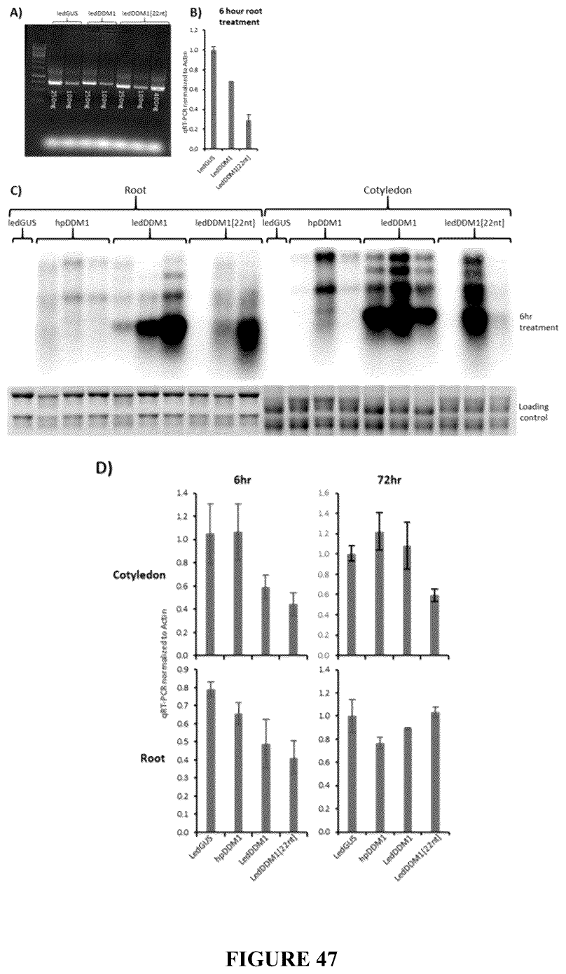

The same assay was used to compare the movement and silencing efficiency of the three different RNAi molecules that were tested. The transcribed ledRNAs were quantified using the Qubit RNA broad spectrum kit followed by validation through visualization on a 1% agarose gel. After the 6 hr treatment by soaking the roots in the RNA solutions, greater reduction of DDM1 mRNA was observed in the cotyledons after root contact with the ledDDM1[Δ22] RNA compared to the ledDDM1[Con] (FIG. 47, panel B).

The analysis was extended to include the symmetric hairpin RNA, hpDDM1[Con], for comparison with the ledDDM1 molecules. The B. napus seedlings were treated as before and both root and cotyledon tissues analysed for uptake and movement of topically applied RNA along with the amount of the target DDM1 mRNA. Movement of the RNA from the root to the cotyledon was observed for all constructs (FIG. 47, panel C). In both roots and cotyledons, the ledDDM1[Δ22] RNA produced significantly greater knockdown of the target mRNA than the ledDDM1[Con] construct at the 6 hr timepoint. At the 72 hr timepoint, the reduction by ledDDM1[Δ22] was still observed within cotyledons (FIG. 47, panel D), showing increased longevity of the silencing effect.

### Example 22: Systemic Movement of RNAi Molecules when Topically Applied to Aerial Parts of Plants

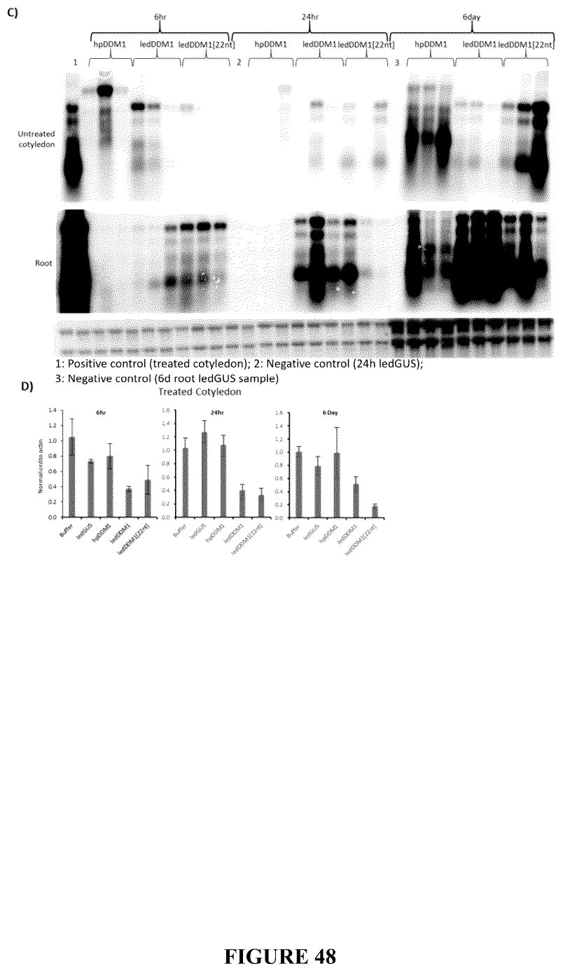

Cotyledons were treated with 2 μg of hpDDM1[Con], ledDDM1[Con] or ledDDM1[Δ22] RNAs using a concentration of 100 μg/ml in a buffer, as before. The RNA solutions were applied as a droplet to one cotyledon of 6-day old B. napus seedlings. Nine seedlings were used for each treatment with 3 seedings collected at each of three different time points, namely 6 hr, 24 hr and 6 days post application. For the 6-day treatment with hpDDM1[Con], an additional 1 μg was applied to the apical meristem. The treatment solutions, including buffer control, all slightly retarded growth of the treated cotyledon, however all plants and treated cotyledons survived. The treated cotyledons, untreated cotyledons and roots were harvested, washed with water and frozen in liquid N2. Northern blots demonstrated the presence of the applied RNA molecules, and therefore systemic movement of, all three of the RNA molecules from the treated cotyledons into the untreated cotyledons and into the roots (FIG. 48). In the treated cotyledons, the target mRNA accumulation was reduced by both of the ledDDM1 molecules at all three time points, with the ledDDM1[Δ22] showing increased silencing at 6 days compared to the hpDDM1 and ledDDM1[Con] molecules (FIG. 48).

It was concluded that the modified RNA molecules, particularly the ledRNA molecules, had greater capability to move systemically in the plants after topical application to aerial organs, in addition to the increased silencing efficiency provided by the asymmetric ledRNA molecules with the Δ22 modification.

### Example 23: Silencing of an Endogenous Fatty Acid Desaturase Gene

Fully expanded Nicotiana benthamiana leaves were painted with a solution containing either ledFAD2[Con] or ledFAD2[Δ22] molecules, or hpFAD2[Con] or hpFAD2[Δ22] molecules. These RNA molecules were produced by in vitro transcription from DNA templates. The ledRNAs were analogous in structure to the ledDDM1 molecules described above, one having the Δ22 modification and the other being the symmetrical control. The hpFAD2[Δ22] molecule was analogous to the hpGUS[Δ22] and hpEIN2[Δ22] molecules described above. After 6 hr, the treated tissues were excised along with an adjacent region not treated with the RNA molecules. Total leaf RNA was extracted from the samples and the levels of FAD2 mRNA quantitated using Northern blot analysis. After 6 hr, the ledFAD2[Δ22] treated leaves exhibited increased gene silencing compared to the ledFAD2[Con] RNA and the ledGUS[Con] RNA used as negative controls. In the adjacent tissue, the ledFAD2[Δ22] showed significant silencing, again demonstrating the improved systemic silencing capability of the asymmetric ledRNA molecules with the Δ22 modification. In similar fashion, the asymmetric hpFAD2[Δ22] hairpin molecules showed increased silencing efficiency compared to the corresponding, symmetrical control hpFAD2[Con] molecules.

### Example 24: Design of Asymmetric RNA Molecules for Reducing Expression of an Essential Plant Gene

### Introduction

Cisneros et al. (2022) tested artificial miRNA (amiRNA) molecules targeting a magnesium chelatase subunit CHL1 Sulfur gene (NbSu) in N. benthamiana plants by agroinfitration of constructs encoding the RNA molecules. They reported that one 21-nucleotide sRNA (amiR-NbSu-2) produced in the infiltrated leaves could move systemically in the plant to reduce expression of the target gene in non-infiltrated leaves. Another 21-nucleotide amiRNA (amiR-NbSu-1) was much less able to produce a silencing effect. That is, the 21/21-mer duplexes produced by Dicer for amiR-NbSu-2, not the precursor RNA molecules or the infiltrated DNA constructs, could move through the plant. This was presumably through phloem (Molnar et al., 2010), over long distances relative to a cellular level, to produce the systemic silencing effect. The reduction of gene expression was observed as a characteristic bleaching phenotype in cells alongside veins, indicating the loss of green chlorophyll in those tissues—the Su gene is essential for chlorophyll synthesis. This phenotype of near-vein chlorosis occurred in upper leaves of the treated plants as well as the infiltrated leaves. That systemic silencing required high levels of amiRNA production in the vicinity of the leaf petioles but was independent of the production of secondary sRNAs from NbSu mRNA. The systemic effect was clearly different to the short-range movement of sRNAs that extends up to 10-15 cells away from the production site (Himber et al., 2003), where the sRNAs move through plasmodesmata connecting the cytoplasm of adjacent cells.

Interestingly, Cisneros et al. (2022) observed that the expression of a 22-nucleotide form of amiR-NbSu-2 from a symmetric precursor pri-miRNA molecule did not induce systemic silencing, despite triggering the biogenesis of 21-nucleotide phased secondary siRNAs from NbSu mRNAs, in contrast to the effective 21-nucleotide amiRNA.

Tang et al. (2010) also targeted the Sulfur gene (NbSu) in N. benthamiana with an amiRNA construct, showing that this gene was suitable as a reporter gene for down-regulation studies based on the visual phenotype of plant tissue bleaching. The present inventors therefore designed a series of asymmetric RNA molecules with multiple variations, each targeting the N. benthamiana Su gene transcript as a reporter gene, in order to identify improved silencing construct designs. These RNA molecules are delivered either by introducing the DNA construct (indirect introduction) into the plant leaves through agroinfiltration or topically as precursor RNA molecules (direct introduction). The present inventors were particularly interested in the ability of these molecules to induce reduction of gene expression in tissues other than those contacted with the precursor RNA molecules.

Design of Asymmetric RNA Molecules with Variations in Structures

A nucleotide sequence (SEQ ID NO: 127) was identified in a N. benthamiana sequence database for a cDNA corresponding to a Sulfur gene RNA transcript, encoding a magnesium-chelatase subunit 1 (CHL1, referred to herein as NbSu). This was from the gene locus lcl|Nbv5.1tr6204879. The encoded amino acid sequence for NbSu is provided herein as SEQ ID NO: 128. A partial nucleotide sequence for a closely related NbSu cDNA was identified as NCBI Accession No. AJ571699.

A 392-nucleotide sequence was selected from the cDNA sequence as a target region, corresponding to nucleotides 463-854 of the 1691 nucleotides of SEQ ID NO: 129. The selected sense sequence lay within about the second quarter of the full-length cDNA sequence, within the protein coding region. The corresponding, fully complementary antisense sequence is provided herein as SEQ ID NO: 130.

Two sets of genetic constructs, each having four constructs, were designed for expression of RNA molecules targeting that region. Each set had three asymmetric RNA molecules with the Δ22 modification in the sense sequence, with or without additional nucleotide substitutions to provide G:U basepairs, and one corresponding symmetric RNA molecule as a control for comparison. The first set of four genetic constructs were for hairpin RNAs with elements in 5′ to 3′ order: 35S promoter-T7 RNA polymerase promoter-sense sequence-loop (intron)-antisense sequence-transcription terminator. The dual promoters provided for in planta expression from the 35S promoter as well as in vitro transcription from the T7 RNA polymerase promoter for each construct, thereby providing for both DNA introduction or direct RNA introduction (topical application of RNA) from the same constructs. These were designated as follows:
Construct 24.1. Conventional, symmetric hairpin with fully canonical basepairing (hpNbSu[Con]).
Construct 24.2. Asymmetric hairpin with every 22nd nucleotide deleted from the sense sequence (hpNbSu[Δ22]).
Construct 24.3. As for Construct 24.2, additionally with 38 A to G substitutions in the sense sequence (hpNbSu[Δ22AG]).
Construct 24.4. As for Construct 24.3, additionally with 24 C to T substitutions in the antisense sequence (hpNbSu[Δ22CT]).

The sense sequence of Construct 24.2 had every 22nd nucleotide deleted, to provide a bulged, non-basepaired ribonucleotide in the antisense sequence after every 21 basepairs in the double-stranded region of the hairpin RNA molecule when produced by transcription. This modification was designed to produce, when cleaved by Dicer, 21/22-mers along the full length of the 392-nucleotide antisense sequence. Constructs 24.3 and 24.4 encoded asymmetric RNA molecules having, in addition to the non-basepaired ribonucleotides of Construct 24.2, multiple G:U basepairs in the duplex region of the hairpin RNA molecules, specifically 38 G:U basepairs out of 392 (9.7%) for Construct 24.3 and 62 G:U basepairs out of 392 (15.8%) for Construct 24.4. The positions of these A to G and C to T substitutions tended to be clustered, where possible, in small regions of 3-11 nucleotides positioned 3′ of each of the nucleotide deletions, aiming to introduce G:U basepairs more toward 5′ end of the 22 nt antisense sRNAs when produced, starting at the duplex-loop junction. Notably, the RNA molecule for Constructs 24.2 and 24.3 had the same 392-nucleotide antisense sequence as the control Construct 24.1, whereas Construct 24.4 had an antisense sequence with 24 C to U substitutions, so that antisense sequence could hybridise to the full length of the selected region of the target transcript but including 24 G:U basepairs. The DNA Constructs 24.2, 24.3 and 24.4 therefore each comprised an imperfect inverted repeat of the selected sequence, with increasing extent of variation from the wild-type sequence from Constructs 24.2 to 24.4.

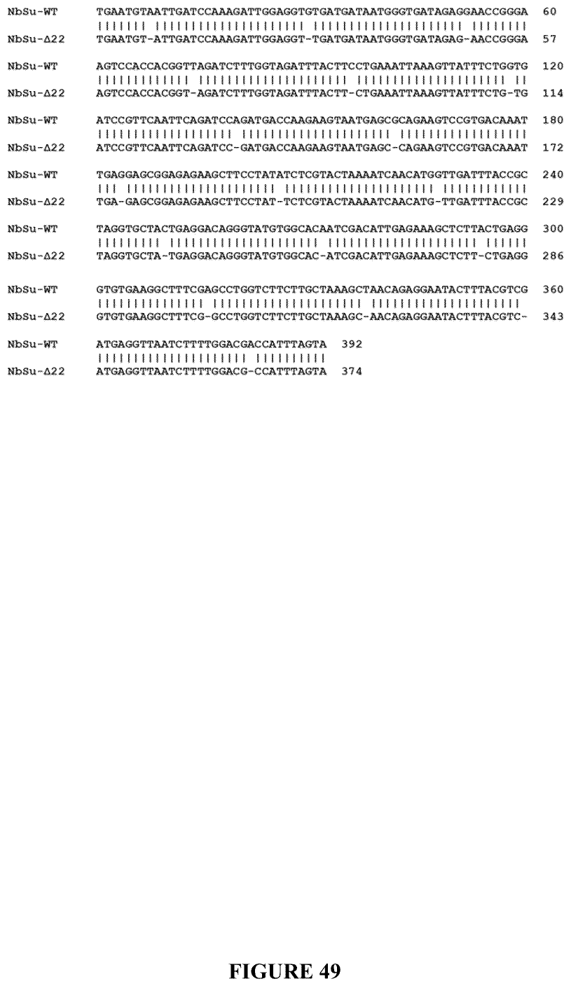

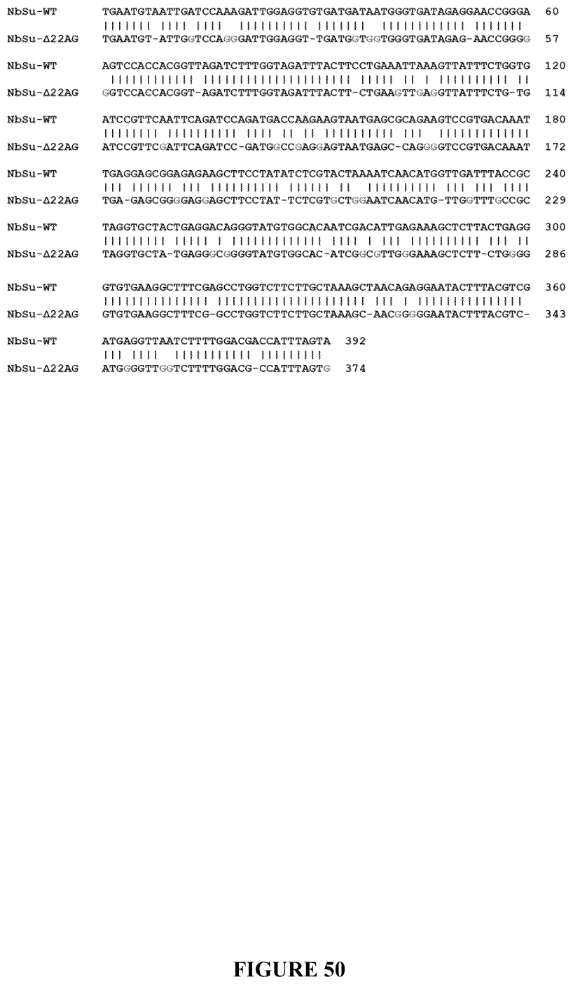

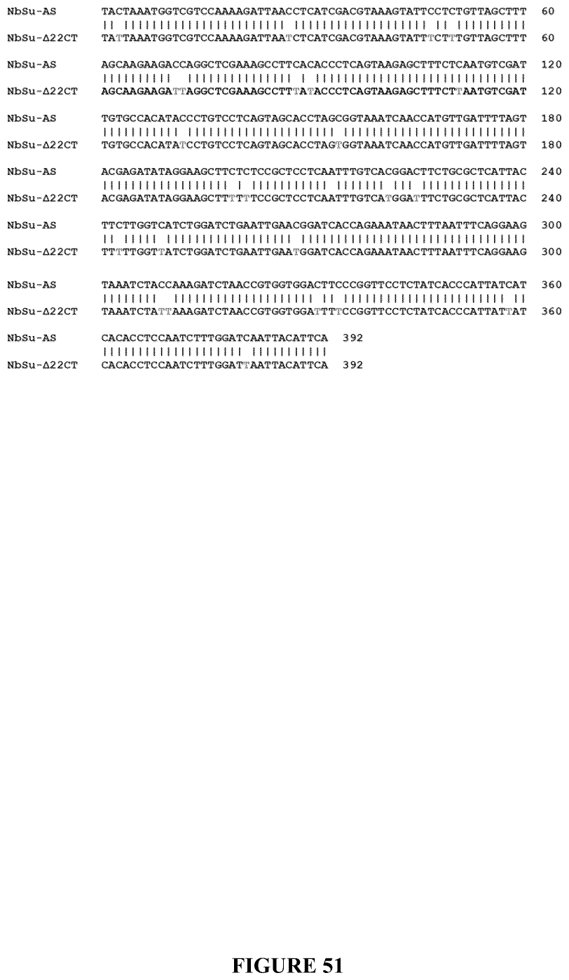

The alignments of the modified sense sequence having the Δ22 modification (SEQ ID NO: 139) in Construct 24.2 and the modified sense sequence having the Δ22 and A to G modifications (SEQ ID NO: 140) in Constructs 24.3 and 24.4 with the wild-type, 392-nucleotide sense sequence (SEQ ID NO: 129) in Construct 24.1 are shown in FIGS. 49 and 50, respectively, showing the positions of the deleted nucleotides and substituted nucleotides. The alignment of the modified antisense sequence (SEQ ID NO: 141) in Construct 24.4, in 5′ to 3′ order, with the unmodified antisense sequence (SEQ ID NO: 130) in Construct 24.1 is shown in FIG. 51, showing the positions of the 24 C to T substitutions.

Each construct had a SmaI restriction enzyme site immediately after the antisense sequence, to be used to terminate the in vitro transcripts by run-off transcription. The hairpin RNA molecules produced in planta by expression from the upstream CaMV 35S promoter will be identical to the corresponding in vitro transcript except for having addition nucleotides attached to the 5′ and 3′ ends as 5′ leader and 3′ trailer sequences, in addition to splicing out of the intron sequence in the loop.

The sequences were adapted for the GoldenGate cloning strategy by adding BsaI restriction sites at the end of each DNA component and assembled using GoldenGate methods. The nucleotide sequences for the RNA coding regions of Constructs 24.1 to 24.4, including the T7 RNA polymerase promoter immediately upstream, are provided as SEQ ID NOs: 131 to 134. The DNA sequences corresponding to the hairpin RNA molecules when produced by in planta transcription are provided herein as SEQ ID NOs 135 to 138, including excision of the intron from the primary transcript, but without the 5′ leader and 3′ trailer sequences coming from the promoter and transcription terminator regions, respectively. Excision of the intron produces a 6-nucleotide linker region joining the 3′ end of the sense sequence to 5′ end of the antisense sequence, forming a small, tight loop in the RNA molecule.

The second set of four constructs contained the same sense and antisense sequences but in the context of ledRNA molecules, i.e. with the duplex enclosed by a loop at each end. These Constructs 24.5 to 24.8 were therefore parallel to Constructs 24.1 to 24.4, as follows:
Construct 24.5. Conventional, encoding a symmetric ledRNA molecule with fully canonical basepairing (ledNbSu[Con]).
Construct 24.6. Encoding an asymmetric ledRNA molecule with every 22nd nucleotide deleted from the sense sequence (hpNbSu[Δ22]).
Construct 24.7. Encoding an asymmetric ledRNA molecule as for Construct 24.6, additionally with 38 A to G substitutions in the sense sequence (hpNbSu[Δ22AG]).
Construct 24.8. Encoding an asymmetric ledRNA molecule as for Construct 24.7, additionally with 24 C to T substitutions in the antisense sequence (hpNbSu[Δ22CT]).

To make the ledRNA constructs, the 392-nucleotide sense sequence was split approximately into two halves, one of 200 nucleotides designated sense-1 (SEQ ID NO: 142) and corresponding to nucleotides 463-662 of SEQ ID NO: 127, and the other of 192 nucleotides designated sense-2 (SEQ ID NO: 143) and corresponding to nucleotides 663-854 of SEQ ID NO: 127. The 392-nucleotide antisense sequence was split at the same position and the two halves, antisense-1 (SEQ ID NO: 145) and antisense-2 (SEQ ID NO: 144), linked by a CCC tri-nucleotide linker. The order of elements in the ledRNA constructs are: 35S promoter-T7 RNA polymerase promoter-sense 2 sequence-linker 2 (intron)-antisense 2 sequence-CCC linker-antisense 1 sequence-linker 1 (intron)-sense 1 sequence-transcription terminator. See FIG. 52 for a schematic map of the linear arrangement of the elements and the folded structure of the ledRNA produced by transcription of the RNA coding region. In this ledRNA molecule, the two sense sequences flank the two antisense sequences which are linked through a CCC tri-nucleotide and could be considered a single antisense sequence with a tri-nucleotide insertion. The linker 1 (intron) and linker 2 (intron) sequences therefore form the two loops sequences in the in vitro transcript, while the introns are expected to be spliced out after in planta transcription. The sense-1 (SEQ ID NO: 146) and sense-2 (SEQ ID NO: 147) sequences of Construct 24.6 had every 22nd nucleotide deleted, in the same positions as in Construct 24.2, to provide a bulged, non-basepaired ribonucleotide after every 21 basepairs in the double-stranded region of the ledNbSu[Δ22] RNA molecule produced from the construct. Constructs 24.7 and 24.8 also had the same Δ22 modifications. Constructs 24.7 and 24.8 encoded asymmetric ledRNA molecules additionally having multiple G:U basepairs in the duplex region of the ledRNA molecules, specifically 38 G:U basepairs out of 392 (9.7%) for Construct 24.7 and 62 G:U basepairs out of 392 (15.8%) for Construct 24.8. These nucleotide substitutions were identical to the corresponding substitutions in Constructs 24.3 and 24.4, respectively. Analogous to the first set of Constructs 24.1 to 24.4, the RNA molecules for Constructs 24.6 and 24.7 had the same antisense-1 and antisense-2 sequences as the control Construct 24.5, whereas Construct 24.8 additionally had 24 C to U substitutions in its antisense sequence, in analogous fashion to Construct 24.4 compared to Construct 24.1. The nucleotide sequences for the modified sense-1 and sense-2 sequences having the Δ22 and A to G modifications are provided herein as SEQ ID NOs: 148 and 149, respectively, and the antisense-2 and antisense-1 sequences having the C to T substitutions are provided as SEQ ID NOs: 150 and 151, respectively.

The nucleotide sequences for the RNA coding regions of Constructs 24.5 to 24.8, including the T7 RNA polymerase promoter in each construct, are provided as SEQ ID NOs: 152 to 155. The DNA sequences corresponding to the ledRNA molecules when produced by in planta transcription are provided herein as SEQ ID NOs 156 to 159, including excision of the two introns from the primary transcript, but without 5′ leader and 3′ trailer sequences coming from the promoter and transcription terminator regions, respectively.

As can be seen from FIG. 52, the hairpin hpNbSu molecules and their counterpart ledNbSu molecules differ only in regard to (i) the nick between the sense-1 and sense-2 sequences i.e. lack of a covalent bond at that position, (ii) the presence of a GGG tri-nucleotide at the 5′ end of the sense-2 sequence and a complementary CCC tri-nucleotide between the antisense-1 and antisense-2 sequences. The transcripts also differ in that (iii) the introns will not be spliced out of the in vitro transcripts but will be spliced from the in planta transcripts. Importantly, the hpNbSu and ledNbSu molecules have the same length, sequence and arrangement of the sense and antisense sequences and so provide almost identical duplex structures in the folded molecules (FIG. 52). These two series of RNA molecules are therefore comparable for effects in reducing Su gene expression in N. benthamiana, as a reporter gene with a visible phenotype when silenced, which is also an essential gene for plant growth.

The genetic sequences were synthesised by a commercial provider and the assembled DNA fragments inserted into the expression vector pART7. The expression cassettes were excised from there and inserted into the T-DNA region of the binary vector pART727 to form the p35S::hpNbSu::ocs-T and p35S::ledNbSu::ocs-T vectors for agro-infiltration and plant transformation. To produce the in vitro transcripts, the construct DNAs were digested with restriction enzyme StuI and used for transcription with T7 RNA polymerase as described in Example 1.

N. benthamiana plants are treated by topical application to the leaves with the in vitro generated transcripts. For in planta transcription, Agrobacterium containing the binary vectors were introduced into leaves by agro-infiltration. This used Agrobacterium tumefaciens strain GV3101 cells transformed with the genetic constructs at an OD600 of 0.8-1.0. The plants were observed for at least three weeks post-treatment to detect bleaching of treated tissues and for the near-vein clearing phenotype in non-treated leaves, indicating reduction of Su gene expression in the non-treated leaves by movement of sRNAs produced from the hairpin or ledRNA molecules. Northern blotting was used to assess the effect of the treatments on the amount of Su mRNA and the production of sRNAs, particularly 22-mer antisense RNAs and the production of secondary 21-mers. The present inventors expect that Constructs 24.2 and 24.6 will be more effective at Su gene silencing in non-treated tissues than Constructs 24.1 and 24.5, respectively. It was observed that all of the tested RNA molecules were able to reduce the amount of the Su mRNA in the infiltrated leaves, as assessed by Northern blotting using RNA samples extracted 3 days post infiltration. Small antisense RNAs of 21-25 nucleotides in length were observed in the RNA preparations from the treated leaves. The size of the sRNAs produced from the ledNbSu[Δ22] and ledNbSu[Δ22ex] RNA molecules appeared noticeably larger at 22 nt compared to those produced from ledNbSu[Con] at 21 nt, but the sRNAs were also less abundant than the 21 nt from ledNbSu[Con]. The sRNA molecules from the ledNbSu[Δ22AG] and ledNbSu[Δ22CT] RNA molecules were even less abundant, indicating reduced processing of those RNA molecules by Dicer. It was also observed that the predominant size of the sRNAs from ledNbSu[Δ22AG] and ledNbSu[Δ22CT] was 22 nt. It was concluded that the shift from 21 nt to 22 nt for the predominant sRNAs also occurred from constructs that had A to G substitutions in the sense strand (e.g. Construct 24.7) and a combination of A to G and C to T substitutions (e.g. Construct 24.8). It was concluded, therefore, that the use of the G:U non-canonical basepairs in the stem of the asymmetric RNA molecules still allowed for production of the longer antisense sRNAs at 22, 23 or 24 nt. That is, the two modifications were compatible.

Two more constructs, Constructs 24.9 and 24.10, were designed which included an additional sense sequence and a complementary antisense sequence from the N. benthamiana Su gene in comparison with Constructs 24.2 and 24.6, respectively. The intent was to lengthen the duplex region of the hairpin and ledRNA molecules by adding a sequence from 3′ untranslated region (3′ UTR) of the Su gene. This is added as an unmodified sense sequence and a fully complementary antisense sequence. The duplex region formed by hybridisation between these will be processed by Dicer to produce 21/21-mer siRNAs, targeting 3′ UTR of the Su mRNA. The aim of this is to cleave off the polyA tail that is added to primary transcripts and thereby increase the efficiency of RDR6/SGS3 producing the complementary strand to the Su transcript, so enhancing the production of secondary, transitive antisense sRNAs.

A 100-nucleotide sequence (SEQ ID NO: 160) was selected as the additional sense sequence from 3′ UTR, corresponding to nucleotides 1482-1581 of SEQ ID NO: 127. The sense sequence and a complementary antisense sequence are inserted in position and orientation downstream of the sense-2 sequence so that the duplex region of the RNA molecules is extended by 100 basepairs. Constructs 24.9 and 24.10 encode the RNA molecules designated as hpNbSu[Δ22ex] and ledNbSu[Δ22ex]. These molecules were tested in the same ways as Constructs 24.1 to 24.8. It was observed that the ledNbSu[Δ22ex] construct was able to induce a vein-clearing or vein-yellowing phenotype in systemic leaves of some of the treated N. benthamiana plants, whereas the ledNbSu[Con] RNA molecule did not produce systemic symptoms, even though it reduced the Su mRNA in the treated leaves. The vein clearing was observed mainly in branch leaves derived from the infiltrated leaf and in younger leaves developing directly above the leaf infiltrated with the ledNbSu[Δ22ex] construct. Essentially the same result was obtained when the experiment was repeated. It was concluded that the sRNA molecules produced from the ledNbSu[Δ22ex] construct were able to move through the plant tissues, probably through the plant vasculature, and induce systemic reduction of gene expression.

The other constructs producing the Δ22 modified product RNA molecules are tested.

### Example 25: Asymmetric Hairpin RNAs Targeting Herbicide Tolerance Genes

In view of the systemic movement of asymmetric RNA molecules, including ledRNA molecules, both from roots to aerial tissues and vice versa, and the transitive gene silencing provided by the Δ22 modification as described in previous Examples herein, the present inventors wished to test the effectiveness of asymmetric RNA molecules in the hairpin and ledRNA formats to reduce the activity of other essential plant genes, thereby acting as a potential herbicide in a weed species. Palmer amaranth (Amaranthus palmeri) is an example of an invasive weed species which has become difficult to control with chemical means due to the emergence and rapid spread of tolerance to herbicides such as glyphosate (Gaines et al., 2010). This weed has a propensity to produce many thousands of seeds per plant and therefore the herbicide tolerant plants have spread quickly, with substantial economic and agronomic consequences (Ward et al., 2013). Tolerance to glyphosate in this species as in many other weed species occurs by a gene amplification mechanism, with amplification of a gene encoding 5-enolpyruvyl-shikimate-3-phosphate synthase (EPSPS), an essential enzyme in the plant that glyphosate binds to and inhibits to provide its herbicide action. Up to 160 EPSPS gene copies were observed in resistant A. palmeri plants (Gaines et al., 2010), so that the massive gene amplification over-expressed the EPSPS enzyme and thereby titrated out the herbicide and reducing its effectiveness. The present inventors therefore designed asymmetric RNA molecules in the hairpin and ledRNA formats to target the RNA transcripts from the EPSPS gene in A. palmeri, as follows.

A nucleotide sequence for the protein coding region of a cDNA for the A. palmeri EPSPS gene was assembled from NCBI Accession No. JX564536; the sequence is provided herein as SEQ ID NO: 161. A 467-nucleotide sequence (SEQ ID NO: 162) was selected as a target sequence from the cDNA sequence, corresponding to nucleotides 60-526 of SEQ ID NO: 161. This sequence was from about 5′ third of the protein coding region. Three hairpin RNA constructs and three analogous ledRNA constructs were designed, designated as follows:
Construct 25.1. Conventional, symmetric hairpin with fully canonical basepairing, in the 5′ to 3′ order of: promoter-sense sequence-intervening sequence (intron)-antisense sequence-transcription terminator (hpEPSPS[Con]).
Construct 25.2. Asymmetric hairpin corresponding to Construct 25.1 except that every 22nd nucleotide deleted from the sense sequence (hpEPSPS[Δ22]).
Construct 25.3. As for Construct 25.2, additionally with 38 A to G or C to T substitutions in the sense sequence (hpEPSPS[Δ22GU]).
Construct 25.4. Encoding a symmetric ledRNA molecule with fully canonical basepairing, using the same sense and antisense sequences as Construct 25.1 (ledEPSPS[Con]).
Construct 25.5. An asymmetric ledRNA molecule identical to Construct 25.4 except that every 22nd nucleotide is deleted from the sense sequence (ledEPSPS[Δ22]).
Construct 25.6. Encoding an asymmetric ledRNA molecule identical to Construct 25.5 except additionally having 38 A to G or C to T substitutions in the sense sequence (ledEPSPS[Δ22GU]).

The sense sequence of Construct 25.2 had 21 single-nucleotide deletions from the sense sequence, evenly spread across the 467-nucleotide sequence, so that about every 22nd nucleotide was deleted, to provide a bulged, non-basepaired ribonucleotide after every 21 basepairs in the double-stranded region of the hairpin RNA molecule when produced by transcription. This modification was designed to produce, when cleaved by Dicer, 21/22-mers along the full length of the 467-nucleotide antisense sequence. Construct 25.3 encoded an asymmetric RNA molecule having, in addition to the non-basepaired ribonucleotides of Construct 25.2, multiple G:U basepairs in the duplex region of the hairpin RNA molecules, specifically 38 G:U basepairs out of 446 basepairs (8.5%). The positions of these A to G and C to T substitutions tended to be clustered, where possible, in small regions of 2-10 nucleotides positioned 3′ of each of the nucleotide deletions, aiming to introduce G:U basepairs more toward 5′ end of the 22 nt antisense sRNAs when produced, starting at the duplex-loop junction. Notably, the RNA molecules for Constructs 25.1-3 all had the same 467-nucleotide antisense sequence, so the antisense sRNA sequences could hybridise to the full length of the selected region of the target transcript. The nucleotide sequence of the modified sense region comprising the 21 single-nucleotide deletions relative to the wild-type sequence is provided herein as SEQ ID NO: 163, for producing hairpin (Construct 25.2) precursor RNA molecules. The nucleotide sequence of a modified sense region comprising 21 single-nucleotide deletions and 38 A to G or C to T substitutions relative to the wild-type sequence is provided herein as SEQ ID NO: 164, for producing hairpin (Construct 25.3) precursor RNA molecules.

Each construct has a SmaI restriction enzyme site immediately after the antisense sequence, to be used to terminate the in vitro transcripts by run-off transcription. The hairpin RNA molecules produced in planta by expression from the upstream CaMV 35S promoter will be identical to the corresponding in vitro transcript except for having addition nucleotides attached to the 5′ and 3′ ends as 5′ leader and 3′ trailer sequences and lacking the intron sequence when spliced out.

Constructs 25.4-25.6 contain essentially the same sense and antisense sequences as Constructs 25.1-25.3 but in the context of encoded ledRNA molecules, i.e. with the double-stranded region enclosed by a loop at each end. To make the ledRNA constructs, the 467-nucleotide sense sequence was split approximately into two halves, one of 244 nucleotides designated sense-1 (SEQ ID NO: 165) and the other of 223 nucleotides designated sense-2 (SEQ ID NO: 166). The 467-nucleotide antisense sequence was split at the same position and the two halves, antisense-1 and antisense-2 linked by a CCC tri-nucleotide linker. This tri-nucleotide linker is added to provide complementary nucleotides to a GGG trinucleotide which is added immediately after a T7 promoter sequence at the 5′ end of the in vitro transcript for increased transcription, thereby minimising non-basepaired ribonucleotides in the double-stranded portion of the ledRNA molecules other than the single-ribonucleotide bulges. The order of elements in the ledRNA constructs are: 35S promoter-T7 RNA polymerase promoter-sense 2 sequence-linker 2 (intron)-antisense 2 sequence-CCC linker-antisense 1 sequence-linker 1 (intron)-sense 1 sequence-transcription terminator. See FIG. 52 for a schematic map of the linear arrangement of the elements and the folded structure of the ledRNA produced by transcription of the RNA coding region. In this ledRNA molecule, the two sense sequences flank the two antisense sequences which are linked through a CCC tri-nucleotide and could be considered a single antisense sequence with a tri-nucleotide insertion.

The sense-1 (SEQ ID NO: 167) and sense-2 (SEQ ID NO: 168) sequences of Construct 25.5 have every 22nd nucleotide deleted, in the same positions as in Construct 25.2, to provide bulged, non-basepaired ribonucleotides after every 21 basepairs in the double-stranded region of the ledEPSPS[Δ22] molecule. All of the basepairs in ledEPSPS[Δ22] are canonical basepairs. In contrast, Construct 25.6 also has the same Δ22 modifications but additionally has multiple G:U basepairs in the duplex region of the ledRNA molecule through A to G and C to T substitutions. These nucleotide substitutions were identical in position to the corresponding substitutions in Construct 25.3 encoding the hairpin RNA, hpEPSPS[Δ22G:U]. The RNA molecules for Constructs 25.4 to 6 are analogous to Constructs 25.1 to 3 in having the same antisense-1 and antisense-2 sequences. The nucleotide sequences for the modified sense-1 and sense-2 sequences having the Δ22 and A to G and C to T modifications are provided herein as SEQ ID NOs: 169 and 170, respectively.

The six constructs are assembled in the same manner as other constructs described above. Each DNA construct is transcribed in vitro to provide precursor RNA molecules for topical application to leaves of A. palmeri plants. In one experiment, the RNA molecules are prepared in a composition with a detergent or wetting agent such as Silwet before being sprayed onto leaves of the plants. In another experiment, the RNA molecules are mixed in a composition with glyphosate before application to the leaves. The plants are monitored visually for several weeks after RNA application, and the EPSPS target gene expression is measured by qRT-PCR.

### Example 26: Asymmetric Hairpin RNAs with Non-Canonical Basepairs that Target Insect Genes

In view of the systemic movement and improved efficacy of asymmetric RNA molecules in the ledRNA format, with the ability to accumulate full-length transcripts in yeast cells such as S. cerevisiae, the present inventors designed a series of constructs to test the combination of features against an insect pest by topical delivery of the RNA molecules after production of the precursor RNA molecules in yeast cells. The Ha6 gene in H. armigera, encoding a synaptic vesicle glycoprotein 2C-like protein (Example 8), was chosen as an exemplary target gene. A chimeric 333-nucleotide target sequence (SEQ ID NO: 171) was designed by joining together two otherwise separate target regions from the cDNA, namely nucleotides 372-527 and 1248-1424 of SEQ ID NO: 52.

Two sets of genetic constructs, each having four constructs, were designed for expression of RNA molecules targeting the chimeric sequence. Each set had two constructs encoding asymmetric RNA molecules with the Δ22 modification in the sense sequence, one with and the other without additional nucleotide substitutions to provide G:U basepairs. Another construct encoded a symmetric RNA molecule having the same A to G and C to T substitutions to provide G:U basepairs but without the Δ22 modification, and the fourth construct encoded a corresponding symmetric RNA molecule with fully canonical basepairing as a control for comparison. The first set of four genetic constructs were for hairpin RNAs with elements in 5′ to 3′ order: yeast promoter-T7 RNA polymerase promoter-sense sequence-loop (intron)-antisense sequence-transcription terminator. The dual promoters provided for expression in yeast cells as well as in vitro transcription from each construct. The constructs could readily be adapted to in planta expression by switching promoters. The constructs were designated as follows:
Construct 26.1. Encoding conventional, symmetric hairpin with fully canonical basepairing using the chimeric sense sequence SEQ ID NO: 171 (hpHa6[Con]).
Construct 26.2. Encoding an asymmetric hairpin as for Construct 26.1 except every 22nd nucleotide deleted from the chimeric sense sequence, all basepairs being canonical basepairs, SEQ ID NO: 172 (hpHa6[Δ22]).
Construct 26.3. As for Construct 26.1, additionally with 51 A to G and 32 C to T substitutions in the chimeric sense sequence, SEQ ID NO: 173 (hpHa6[GU]).
Construct 26.4. As for Construct 26.2, additionally with 51 A to G and 32 C to T substitutions in the chimeric sense sequence, SEQ ID N: 174 (hpHa6[Δ22GU]).

The chimeric sense sequence of Construct 26.2 had every 22nd nucleotide deleted, to provide a bulged, non-basepaired ribonucleotide in the antisense strand after every 21 basepairs of the double-stranded region of the hairpin RNA molecule when produced by transcription. This Δ22 modification was designed to produce, when cleaved by Dicer, 21/22-mer product RNA molecules along the full length of the 333-nucleotide chimeric antisense sequence. Construct 26.3 had 51 A to G and 32 C to T substitutions, a total of 83/333 nucleotide substitutions (25%), spread evenly along the chimeric sense sequence to provide G:U basepairs in the hairpin RNA. Construct 26.4 encoded asymmetric RNA molecules with the Δ22 modification having, in addition to the non-basepaired ribonucleotides of Construct 26.2, multiple G:U basepairs in the duplex region of the hairpin RNA molecules as for Construct 26.3. This construct thereby combined the two types of modifications in the hairpin RNA context. Notably, the RNA molecules for Constructs 26.2 to 26.4 all had the same 333-nucleotide chimeric antisense sequence as the control Construct 26.1 so that antisense sRNAs produced from all four RNA molecules by Dicer would all hybridise fully to the selected regions of the Ha6 target transcript.

The order of the sequences in each hairpin construct were: yeast TEF promoter-T7 RNA polymerase promoter-sense sequence-loop (intron)-antisense sequence-transcription terminator. Each construct had a SmaI restriction enzyme site immediately after the antisense sequence for use to terminate the in vitro transcripts by run-off transcription. The hairpin RNA molecules produced by expression from the upstream yeast TEF promoter will be identical to the corresponding in vitro transcript except for having addition nucleotides attached to the 5′ and 3′ ends as 5′ leader and 3′ trailer sequences and lacking the intron sequence after splicing.

The second set of four constructs contain essentially the same chimeric wild-type or modified sense and antisense sequences but in the context of ledRNA molecules, i.e. with the duplex enclosed by a loop at each end. These Constructs 26.5 to 26.8 were therefore parallel to Constructs 26.1 to 26.4, as follows:
Construct 26.5. Encoding a symmetric ledRNA molecule with fully canonical basepairing (ledHa6[Con]).

Construct 26.6. Encoding an asymmetric ledRNA molecule as for Construct 26.5 except every 22nd nucleotide is deleted from the sense-1 and sense-2 sequences (hpHa6[Δ22]).
Construct 26.7. Encoding a symmetric ledRNA molecule as for Construct 26.5, except with 21 A to G and 21 C to T substitutions in the sense-1 sequence and 30 A to G and 11 C to T substitutions in the sense-2 sequence (ledHa6[GU]), the same substitutions as in Construct 26.3.
Construct 26.8. Encoding an asymmetric ledRNA molecule as for Construct 26.6, except additionally with 21 A to G and 21 C to T substitutions in the sense-1 sequence and 30 A to G and 11 C to T substitutions in the sense-2 sequence (ledHa6[Δ22GU]).

To make the ledRNA constructs, the two sense sequences that made up the chimeric sense sequence (SEQ ID NO: 172) were kept as separate sense sequences, one of 156 nucleotides designated sense-1 (SEQ ID NO: 175) and corresponding to nucleotides 372-527 of SEQ ID NO: 52, and the other of 176 nucleotides designated sense-2 (SEQ ID NO: 176) and corresponding to nucleotides 1248-1424 of SEQ ID NO: 52. The two fully complementary antisense sequences designated antisense-1 and antisense-2 were linked by a CCC tri-nucleotide linker and used in all four ledRNA constructs 26.5-26.8. The order of elements in the ledRNA constructs are: yeast TEF promoter-T7 RNA polymerase promoter-sense 2 sequence-linker 2 (intron)-antisense 2 sequence-CCC linker-antisense 1 sequence-linker 1 (intron)-sense 1 sequence-transcription terminator. See FIG. 52 for a schematic map of the linear arrangement of the elements and the folded structure of the ledRNA produced by transcription of the RNA coding region. In these ledRNA molecules, the two sense sequences flank the two antisense sequences. The sense-1 (SEQ ID NO: 177) and sense-2 (SEQ ID NO: 178) sequences of Construct 26.6 had every 22nd nucleotide deleted, in the same positions as in Construct 26.2, to provide a bulged, non-basepaired nucleotide after every 21 basepairs in the double-stranded region of the ledHa6[Δ22] molecule. Construct 26.8 also had the same Δ22 modification. Constructs 26.7 and 26.8 encoded ledRNA molecules additionally having multiple G:U basepairs in the duplex region of the ledRNA molecules, totalling 83/333 nucleotides of the antisense sequences involved in G:U basepairs. The nucleotide sequences for the modified sense-1 and sense-2 sequences having the A to G and C to T substitutions are provided herein as SEQ ID NOs: 179 and 180, respectively, and the sense-1 and sense-2 sequences having the A to G and C to T substitutions in addition to the Δ22 modifications are provided herein as SEQ ID NOs: 181 and 182, respectively.

The genetic sequences were synthesised by a commercial provider and the assembled DNA fragments inserted into a yeast expression vector based on a 2-micron replicative region as a multi-copy plasmid vector. The transcribed regions including the T7 RNA polymerase promoter were inserted between an upstream TEF1 promoter and a downstream CYC1 transcription terminator to form the pTEF1::hpHa6::CYC1 terminator and pTEF1::ledHa6::CYC1 terminator constructs. This expression system provides for strong, constitutive expression of the RNA molecules in S. cerevisiae cells. To produce the in vitro transcripts, each construct DNA is digested with restriction enzyme SmaI and used for transcription with T7 RNA polymerase as described in Example 1.

Yeast cells such as strain BY4742Δrrp6Δski3 (WO2019/213761) are transformed with the plasmid constructs and transformed cells selected. This yeast strain carries deletions of two genes, RRP6 and SKI3, and has been shown to accumulate significantly more RNAi molecules than the corresponding non-mutated strain (WO2019/213761). Confirmed transformants were cultured in standard growth medium and the cells harvested either before or after a heat treatment to kill the cells. Non-transformed BY4742Δrrp6Δski3 cells were cultured as a control. The harvested cells are spray dried into fine powders. These cells are added to an artificial diet at a range of amounts and fed to H. armigera larvae in vitro. The growth and survival of the treated larvae are monitored. Leaves of tobacco and pakchoi plants are treated by topical application of the in vitro generated transcripts. In parallel, other plants are dusted with dried powder of the yeast cells. H. armigera larvae are applied to the treated leaves and observed for up to two weeks. Reduced growth, measured by reduced length and weight gain, and increased mortality is expected to be observed in larvae that ingest yeast cells expressing the RNA molecules, in particular for the molecules having the Δ22 modification and for the ledHa6 molecules relative to the conventional hairpin RNA.

### Example 27: Asymmetric Hairpin RNAs Comprising Additional Target Sequences

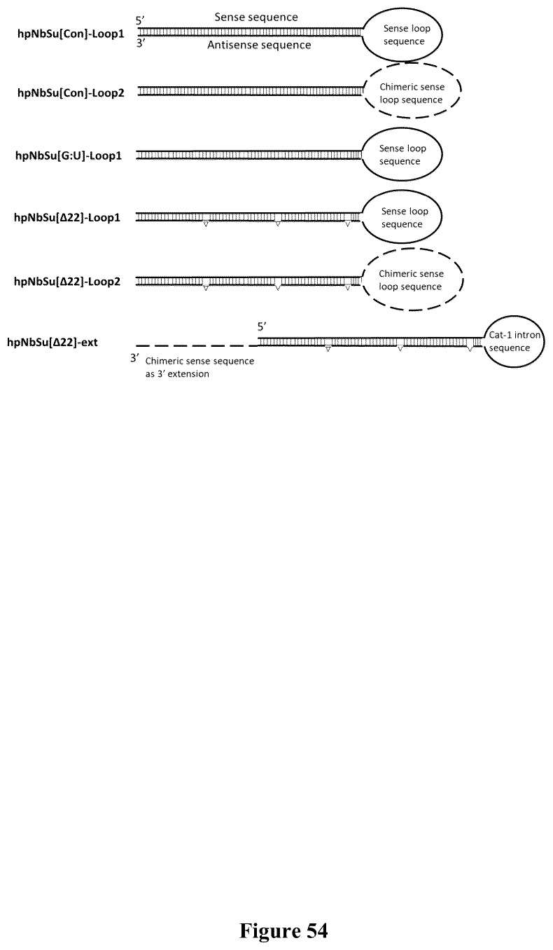

In Example 24, the constructs designated as 24.9 and 24.10 contained a 100 nt unmodified sense sequence from the target RNA transcript, corresponding to a 3′ UTR region of the target transcript, in addition to the modified sense sequence, and a complementary antisense sequence that was fully basepaired to the 100 nt sense sequence by canonical basepairs. This additional dsRNA region essentially extended the duplex structure and was designed to be cleaved by Dicer to provide 21/21-mer siRNAs, intending that the 21-mer antisense sRNAs would result in cleavage of the target RNA transcript in its 3′ UTR and induce more secondary siRNAs to be produced. The present inventors conceived of several variations of this concept of adding target-related sequences to the chimeric RNA molecules, for example adding single-stranded sequences into the loop sequence, or as the loop sequence, of a hairpin RNA structure, or into one or two of the loops in a ledRNA structure. By replacing an intron sequence in the loop of a hairpin RNA with a non-intron sequence, the inventors intended to compare hairpin RNAs with an intron in the loop to hairpin RNAs lacking the intron, for silencing efficiency and determine the extent of production of secondary siRNAs. Some of these concepts for variant chimeric RNA molecules are shown schematically in FIG. 54.

In one variant, the loop sequence of Construct 24.1 encoding hpNbSu[Con] containing a Cat-1 intron (SEQ ID NO: 131) was substituted with an unmodified 400 nt sense sequence from the target NbSu transcript, corresponding to nucleotides 855-1254 of SEQ ID NO: 127, to generate Construct 27.1 (SEQ ID NO: 183), encoding the chimeric RNA designated hpNbSu[Con]-loop1 (FIG. 54). That loop sequence of 400 nt was contiguous with, and downstream of, the sense sequence used to form the duplex, i.e nucleotides 463-854 of SEQ ID NO: 127. This chimeric RNA molecule hpNbSu[Con]-loop1 therefore differed from hpNbSu[Con] only in its loop sequence, importantly lacking an intron.

In another, analogous variant, the loop sequence of Construct 24.1 was substituted with the unmodified 400 nt sense sequence from the target NbSu transcript and the sense sequence for the duplex was modified with the same A to G and C to T substitutions as in Construct 24.3, to generate Construct 27.3, encoding the chimeric RNA designated hpNbSu[G:U]-loop1 having G:U basepairs (9.7%) in the duplex region (FIG. 54) as well as the 400 nt sense sequence as the loop. Again in analogous fashion, the loop sequence of Construct 24.2 (SEQ ID NO: 132) was substituted with the unmodified 400 nt sense sequence from the target NbSu transcript, to generate Construct 27.4 (SEQ ID NO: 185), encoding the chimeric RNA designated hpNbSu[Δ22]-loop1 having the Δ22 modification in the duplex as well as the 400 nt sense sequence as the loop.

These three variant constructs are therefore used in planta and in topical-application assays for their silencing efficiency in comparison with their progenitor constructs to test the effect of sense sequences as loops rather than the intron-containing loops. The present inventors predict that the RNA molecules having the loop sequences derived from the target transcripts will provide increased silencing efficiency and increased levels of secondary siRNAs compared to the corresponding RNA molecules lacking the target-related loop sequences and instead having an intron sequence in the loop. In alternative variations, the inventors conceived of using antisense sequences from the target RNA transcripts as the loop sequence for the RNAi molecule, or a combination of sense and antisense sequences such that at least some of these sequences remained single-stranded in the RNAi molecule.

In another set of variants, a chimeric loop sequence was used having a second sense sequence (referred to as a seed region) incorporated into the loop sequence. This seed region corresponded to a 100 nt region from within the sense sequence used to form the duplex of the RNA molecule. The inventors considered that the antisense sRNAs produced by Dicer from the duplex region of the RNA molecules would hybridise to this seed region in the single-stranded loop region and thereby stimulate binding of the RDR6/SGS3 complex to the cleaved RNA molecule and increase production of secondary siRNAs. This was considered to be particularly favourable when the duplex comprised the Δ22 modification, providing increased production of 22 nt antisense sRNAs that could hybridise to the seed region of the loop. To make these constructs, a 100 nt seed region corresponding to nucleotides 517-616 of SEQ ID NO: 127 was selected and added to the loop sequence of Construct 27.1, generating Construct 27.2 (SEQ ID NO: 184), encoding the symmetric RNA molecule designated hpNbSu[Con]-loop2, thereby having the 500 nt chimeric loop sequence. In analogous fashion, the 500 nt chimeric loop sequence was used to replace the loop sequence of Construct 24.2 (SEQ ID NO: 132), generating Construct 27.5 (SEQ ID NO: 186) encoding the asymmetric molecule designated hpNbSu[Δ22]-loop2 (FIG. 54).

The inventors also conceived that the chimeric sense sequence including the seed region could be inserted into different regions of the RNA molecule, in particular attached to its 5′ or 3′ end rather than in the loop. In another variant that was designed, the chimeric 500 nt sense sequence was inserted immediately 3′ of the antisense sequence of Construct 24.2, generating Construct 27.6 (SEQ ID NO: 187), encoding the asymmetric molecule designated hpNbSu[Δ22]-ext.

Constructs encoding ledRNA molecules having the same type of modified loop sequences and modified duplex regions were also contemplated, with or without seed regions, including where the duplex regions of the first and second components targeted different target RNA transcripts and where the first and second loop sequences corresponded to regions from the different target transcripts.

These constructs are inserted into a binary vector for introduction into plant cells for in planta expression. They are also transcribed in vitro to produce RNA molecules that are applied topically to plant tissues. These constructs are also inserted into a yeast vector for production in yeast cells. The resultant constructs and RNA preparations are tested using the methods described in Example 24.

### Example 28. In Planta Expression of Modified RNA Molecules is Effective to Reduce Sucking Insect Gene Activity

### Introduction

To test the potential of G:U modified RNA molecules produced in planta to control sap-sucking insects, the present inventors selected the green peach aphid (Myzus persicae) as a model sap-sucking insect. Aphids (Hemiptera: Aphididae) are exclusively phloem feeders, sucking sap especially from young growing tissues in a plant. M. persicae is a serious, worldwide pest for a wide range of plant species, feeding on plant tissues from more than 40 plant families (Annis et al., 1981). This group of insects not only causes reduced plant growth through water stress and wilting but is also a vector for diseases by transmitting a variety of plant viruses. Aphids can reproduce to high densities through either sexual or asexual reproduction.

Mao and Zeng (2014) expressed a conventional hairpin RNA in transgenic tobacco plants where the hairpin RNA had a 427-nucleotide sequence corresponding to a region of a hunchback gene of M. persicae. In their feeding tests, no effect was observed after feeding neonate aphids for 7 days and only a modest decrease of about 18% was observed in the aphid population per plant and the aphid biomass (mg) per plant after 14 days. Aphid lethality and defective phenotypes of hunchback suppression were not observed. In another study, Nilaparvata lugens nymphs were fed on rice plants expressing dsRNAs of three target genes (Zha et al., 2011). RNA interference was triggered but lethal phenotypic effects were also not observed after feeding on the plants expressing dsRNA. The present inventors therefore tested modified RNA molecules for protecting plants against aphids, in particular RNAi molecules having G:U basepairs in the dsRNA region, looking for improved control of the pests.

Design of Hairpin RNAs and their Expression in Planta

The hunchback gene of M. persicae (MpHb; Accession No. XM_022313819) was selected as the target gene to test the G:U modified hairpin RNA and ledRNA molecules for comparison to the corresponding canonically-basepaired hairpin or ledRNAs. The MpHb gene encodes a zinc-finger type transcription factor which is involved in embryo development. Mutation or silencing of a gap gene such as MpHb causes the loss of contiguous body segments which can lead to lethality of the insects in the next generation, for example in pea aphids (Acyrthosiphon pisum) (Mao and Zeng, 2014). A cDNA sequence for a gene encoding the Hb protein was identified in a database, provided herein as SEQ ID NO: 188 (Accession No. XM_022313819), encoding a Hb amino acid sequence presented herein as SEQ ID NO: 189. Two hairpin constructs were designed and made: one encoding a hairpin RNA having only canonical basepairing in its dsRNA region (hpMpHb[Con]) and the second (hpMpHb[G:U]) which was the same except for having 147 cytosines in the sense sequence of the DNA construct replaced with thymidines, to allow for G:U basepairing (147/500=29.4% G:U basepairs) in the dsRNA region of the transcript. Both hairpin RNAs thereby had a dsRNA region or stem of 500 bp comprising an antisense sequence that was fully complementary to a target region of the MpHb mRNA sequence, namely nucleotides 1709-2208 of the Hb cDNA sequence (SEQ ID NO: 188), and a chimeric antisense sequence for the loop of 240 bp, comprising the complement of nucleotides 1549-1708 of SEQ ID NO: 188 joined to the complement of nucleotides 2209-2291 of SEQ ID NO: 188. Both DNA sequences were synthesized and cloned into pART7 and then pART727 to form the p35S::hpMpHb::Ocs-T binary vectors for plant transformation. The nucleotide sequences encoding the hairpin RNAs are provided herein as SEQ ID NOs: 190 and 191. Both sequences had an upstream T7 RNA polymerase promoter sequence and a downstream SmaI restriction site to be used for in vitro transcription to produce hairpin RNA.

Constructs to make the corresponding ledRNA RNA molecules, with or without G:U basepairing, are also made.

In an initial experiment to test the expression of the two hairpin constructs in plant cells and processing of the transcripts, the constructs were separately introduced into N. benthamiana leaves by Agrobacterium-mediated infiltration, without the presence of any silencing suppressor such as V2 or CMV 2b. A hpGFP construct that had no corresponding target sequence in the aphids was separately infiltrated as a negative control. There were three replicate infiltrations per construct. Two days after the infiltration, each infiltrated area of the N. benthamiana leaves was infested with five aphid nymphs at the 2 or 3 instar stage of development using a clip cage to prevent movement beyond the infiltrated tissues. On the 11th day following aphid infestation, the number of surviving aphids and nymphs produced in each cage were recorded. Compared to the control construct encoding hpGFP (4.8 nymphs per adult), the expression of hpMpHb[G:U] significantly reduced the aphid reproduction rate (1.47 nymphs per adult, 70% reduction). In contrast, hpMpHb[Con] resulted in only a moderate reduction in aphid reproduction (3.52 nymphs per adult, 27% reduction) relative to the negative control.

Following the transient expression experiment, the hpMpHb[Con] and hpMpHb[G:U] constructs were separately introduced into Arabidopsis thaliana Col-0 ecotype to produce stably transformed plants having the T-DNAs integrated into the nuclear genome. The hpGFP construct was also used in a separate transformation as the negative control. For each construct, two independent Arabidopsis T2 lines with a single T-DNA insertion, based on a 3:1 segregation ratio using kanamycin selection, were selected for the aphid assays. For each line, three plants were used. One mature leaf of each plant was infested with five nymphs at the 2 to 3 instar stage of development using clip cages as before. On the 10th day following aphid infestation, the number of surviving aphids and nymphs produced in each cage were recorded. On the control Arabidopsis plants expressing hpGFP, the average number of nymphs per adult was 7.15. In contrast, on the hpMpHb[G:U] lines, the average number of nymphs per adult decreased to 3.45, a 52% reduction in aphid reproduction compared to that on the control plants. Similar to the results with transient expression in N. benthamiana, the Arabidopsis plants expressing hpMpHb[Con] showed only a moderate reduction in aphid reproduction with 5.79 nymphs per adult, a 20% reduction compared to the control plants. It was concluded that (i) the G:U modified dsRNA molecules were more effective in reducing aphid reproduction than the fully-canonically basepaired dsRNA, and (ii) the N. benthamiana transient expression system gave the same result and was predictive of the results achieved in stable transformed plants expressing the same construct.

### Example 29. Modified RNA Molecules Targeting Fall Armyworm

### Introduction

In view of the positive results obtained using the G:U modified RNA molecules against H. armigera when expressed in planta (Examples 4-11 herein), the present inventors sought to demonstrate similar effectiveness against another Lepidopteran insect pest, Spodoptera frugiperda, also known as the fall armyworm (FAW). FAW is one of the most destructive insect pests and significantly constrains crop production worldwide, resulting in severe losses to maize, rice, wheat, cotton, sorghum, soybean and other crops, particularly in North and South America, Asia and Africa. The present inventors therefore designed hairpin RNA molecules against eight different target genes in FAW, each of the molecules having about 25% G:U basepairing in their dsRNA regions by C to T substitutions in the sense sequences of the constructs encoding the RNA molecules. These were first tested by feeding FAW larvae on corn leaves painted with in vitro-transcribed RNA preparations.

### Design and Production of Modified RNA Molecules Against Different Sf Target Genes

Eight FAW genes of various biological functions were chosen as candidate target genes (Table 5). The nucleotide sequences of the cDNAs for the genes and the encoding proteins are provided herein as SEQ ID NOs: 192 to 207. It was noted that the RNA transcripts of some of these genes were subject to variable splicing and therefore produced multiple different mRNAs and encoded multiple, related proteins. To make the constructs encoding the modified hairpin RNAs, a 300-nucleotide fragment from a S. frugiperda cDNA sequence corresponding to each gene was selected as the target sequence and used to produce an interrupted inverted repeat in each construct to provide for the stem-loop structure. A 146-150-nucleotide sequence from a region immediately downstream of the same cDNA relative to the targeted region in the case of the constructs for the Sf1-Sf7 hairpins, or immediately upstream of the target region in the case of the Sf8 construct, was used as the loop sequence in the antisense orientation. The constructs thereby each had an order of components as promoter-antisense sequence-antisense loop-modified sense sequence-terminator. The sense sequence in each case was modified by replacing all cytidine (C) nucleotides with thymidine (T) nucleotides, while the antisense sequence remained unchanged to ensure full complementarity by canonical basepairing with the region of the target gene mRNA for gene silencing. The DNA fragments were synthesized by a service provider and cloned into the pART727 expression vector by KpnI/SpeI digestion and ligation in order to provide an upstream CaMV 35S promoter for expression and a downstream Ocs-3′ polyadenylation/transcription terminator sequence. Each DNA construct also had a T7 RNA polymerase promoter to provide for production of the hairpin RNAs by in vitro transcription. The nucleotide sequences of the DNA fragments encoding the modified hairpin RNA molecules, including the T7 RNA polymerase promoter, are provided as SEQ ID NOs: 208 to 215 herein. The selected target sequences, each of 300 nt, and encoded polypeptides were as follows:
Sf1: nucleotides 891-1190 of SEQ ID NO: 192 (Synaptic vesicle glycoprotein 2B)
Sf2: nucleotides 182-481 of SEQ ID NO: 194 (v-ATPase catalytic subunit A)
Sf3: nucleotides 2851-3150 of SEQ ID NO: 196 (nuclear hormone receptor HR3)
Sf4: nucleotides 392-691 of SEQ ID NO: 198 (attacin-A-like)
Sf5: nucleotides 1627-1926 of SEQ ID NO: 200 (Ecdysone receptor)
Sf6: nucleotides 170-469 of SEQ ID NO: 202 (Coatomer subunit beta)
Sf7: nucleotides 263-562 of SEQ ID NO: 204 (beta-1,3-glucan-binding protein)
Sf8: nucleotides 289-588 of SEQ ID NO: 206 (troponin C)

To prepare the modified hairpin RNAs for in vitro feeding assays, a TranscriptAid T7 High Yield Transcription kit was used. DNA templates for the in vitro transcription reactions were amplified from the hairpin constructs by PCR with a 35S promoter forward primer and an Ocs 3′ terminator reverse primer. The amplified DNA fragments were purified through a Qiagen PCR clean-up column and used for in vitro transcription following the manufacturer's instructions. Each 20 μL reaction included 4 μL of 5×TranscriptAid reaction buffer, 8 μL of ATP/CTP/GTP/UTP mix, 1 μg of purified PCR fragment and 2 μL of TranscriptAid enzyme mix. The reaction mixtures were incubated at 37° C. for 3 hours. After transcription, 2 μL of RNase-free DNase I was added to each reaction mixture and incubated at 37° C. for 15 min to remove the template DNA. Each mixture was made up to 150 μL with 115 μL DEPC water, 15 μL of 3 M sodium acetate, pH 5.2, extracted with 150 μL phenol/chloroform, and the RNA precipitated with 2.5 volume of absolute ethanol at −20° C. for 1 hr. Precipitated RNA was pelleted by centrifugation at 13,000 rpm for 15 min, washed with 75% ethanol, dried and resuspended in 50 μL of DEPC-treated water. The RNA concentration was determined based on band intensity after agarose gel electrophoresis with a GeneRuler 1 kb DNA ladder as the concentration reference.

To conduct in vitro feeding assays using the hairpin RNA preparations, a leaf-painting feeding system was established. In brief, corn leaves were excised and cut into 12-15 cm long segments and 10 μg of a transcript preparation in 0.5 mM EDTA and 0.03% Silwet was painted onto the excised corn leaf segments with a soft paint brush. Painted leaves were left to air dry and placed in a 9 cm Petri dish with a thin agar layer at the bottom.

In an initial experiment to test stability of the hairpin RNA on the leaf surfaces, some of the transcribed and purified hpSf3[G:U] RNA was applied to the surface of excised corn leaves, which were then air-dried. First-instar S. frugiperda larvae were applied to some of the leaves, whereas other leaves had no larvae applied. The leaves with or without applied larvae were incubated at 25±1° C., 50±10% relative humidity, with a 14:10 day-night photoperiod. Samples were taken from the leaf segments after 1, 2 or 3 days and RNA extracted and subjected to northern blot hybridisation using a radiolabelled sense strand as probe to detect the antisense strand of the hpSf3[G:U] molecules. An aliquot of the in vitro transcript was used as a size control in the northern blot analysis. It was observed that the band pattern and intensity of the hairpin RNA applied to the leaf surfaces remained the same from 1-3 days in the absence of larval feeding, indicating that the RNA molecules were stable on the excised corn leaf surface. For leaves where larvae were applied, the band pattern remained the same over the three days but the band intensity decreasing with time, suggesting some degradation.

To test the effect of each of the hairpin RNA preparations on the larvae after feeding, one-day-old S. frugiperda (neonate) larvae were placed on the RNA-coated leaves and allowed to feed using the incubation conditions as mentioned above. The leaf segments were replaced every day with fresh, newly painted leaves and the larvae transferred gently with a soft paint brush to the fresh leaves. Control leaves were either painted with an unrelated hpGFP RNA or buffer only (no RNA). RNA was isolated using the Trizol reagent from 10 larvae per treatment after 3 or 6 days of feeding. To test for the presence and integrity of the RNA molecules in the fed larvae, samples of the RNA were analysed by northern blot hybridisation analysis and qRT-PCR using a radiolabelled probe corresponding to the 300 nt of each dsRNA region, to specifically detect antisense sequences derived from the hairpin RNA stem.

Specific, small antisense RNA molecules derived from the Sf3 hairpin RNA, much smaller than the full-length hairpin RNA, were readily detected in the larvae that fed on the hpSf3[G:U]-painted corn leaves at both 3 and 6 days. While the majority of the hybridizing signals were small in size, indicative of partial degradation or cleavage of the hairpin RNA molecules, some hybridisation was observed to full-length RNA molecules. This result indicated clearly that the G:U hpRNA was delivered into the insect body via oral feeding and could be detected.

Further, to evaluate the expression level of the target genes after ingesting the hairpin RNAs, northern blot analysis was used to assay for effects on the target mRNA transcripts in the larvae. The elongation factor 1-alpha-like (EF1α) gene was used as the reference transcript for normalisation. In an initial experiment, the northern blot results with the antisense probe to detect target mRNA transcripts showed that the Sf3 and Sf7 genes were clearly downregulated after 3 days of feeding with the hpSf3[G:U] and hpSf7[G:U] RNAs, respectively, showing strong (Sf3) or substantial (Sf7) reduction in mRNA abundance. However, after 6 days of feeding, the transcript levels appeared to have recovered, suggesting that gene silencing occurred mainly at the early developmental stages of the insect larvae. The northern blot hybridization result was confirmed by qRT-PCR analysis, showing approximately 95% downregulation of the Sf3 target mRNA and approximately 25% downregulation of the Sf7 mRNA after 3 days of feeding. There was no reduction observed when using the hpGFP[G:U] control RNA or buffer alone.

The genetic expression constructs were introduced into Agrobacterium tumefaciens strain AGL1 and introduced into plant leaves using standard techniques (Example 1). This provides for in planta expression of the constructs to produce the hairpin RNAs. Genetic constructs were also made having a maize Ubi promoter linked to the RNA coding regions for expression in rice plants. These constructs were used to produce transgenic rice plants by Agrobacterium-mediated transformation. Leaves from the transgenic plants are analysed by northern blot hybridisation to assess the amount of RNA molecules that accumulate, in particular the amount of full length transcript, and used in feeding assays with FAW larvae. Plants that show resistance to the insects are selected.

The inventors considered that the RNA molecules containing the G:U basepairs could be improved by a more even distribution of the G:U basepairs along the dsRNA region of each of the hairpin molecules. This was achieved by (i) substituting only some of the C nucleotides to T, in particular substituting only some but not all of multiple contiguous C nucleotides, and (ii) some A to G substitutions in the sense sequence of the genetic construct, to aim for one G:U basepair about every 4-6 basepairs in the dsRNA region i.e between 16-25% G:U basepairs in the duplex.

Analogous constructs are made to express, in the plant cells, RNA molecules having an ledRNA structure, with or without G:U basepairing and with or without the asymmetric modification (Δ22, Δ23 or Δ24 modification, or combinations thereof). The analogous constructs are also expressed in yeast cells to produce the RNA molecules, which cells are then incorporated into an artificial diet for the S. frugiperda larvae or which are dusted onto corn leaves to be ingested by the larvae. These methods are suitable for rapid-throughput screening of candidate target genes in the pests to identify the most effective target genes, or combinations of the target genes, for control of the pests.

TABLE 5
Eight selected candidate target genes in S. frugiperda.

| Gene | Common | Gene ID | Tissue | Biological |
|---|---|---|---|---|
| designation | gene name | (Accession No.) | expression | function |
| Sf1 | Synaptic | LOC118274261 | Nervous | Cellular transport |
| | vesicle | (XM_050705251) | system, gut | and trafficking |
| | glycoprotein | | | |
| | 2B | | | |
| Sf2 | V-ATPase- | LOC118267501 | Midgut | |
| | A | (XM_035581534) | tissues | |
| Sf6 | Coatomer- | LOC118275640 | Gut, neural | |
| | beta | (XM_035594799) | tissues | |
| Sf3 | Hormone | LOC118279815 | Epidermis, | Development and |
| | receptor | (XM_050702331) | integument | hormonal |
| | HR3 | | | regulation |
| Sf5 | Ecdysone | LOC118267067 | Whole body | |
| | receptor | (XM_035580825) | | |
| Sf4 | Attacin-A- | LOC118270353 | Fat Body, | Immune response |
| | Like | (XM_035585918) | gut | |
| Sf7 | Beta-1,3- | LOC118281636 | Fat Body, | |
| | glucan | (XM_035602265) | gut | |
| | binding | | | |
| | protein | | | |
| | [symbol] | | | |
| Sf8 | Troponin C | LOC118272276 | Muscle | Muscle function |
| | | (XM_035588676) | tissues | and movement |

[symbol]

indicates data missing or illegible when filed

### Example 30: Precursor RNA Molecules that Target Genes in Fusarium

The numerous fungal species of the genus Fusarium, in the Division Ascomycota, are soil-borne, filamentous fungi that, for many species, cause various root and stem rots, vascular wilt or fruit rot of plants. Fusarium species, collectively, are pathogenic for more than 100 plant species such as cotton, cereals, sweet potato, tomato, legumes such as cowpea, melons, potato, sugarcane, garden bean and bananas, although many other Fusarium species are harmless endophytes or saprobes. Infected plants are often stunted with pale green to yellow leaves which wilt, wither, die and drop off progressively upward from the stem base. Dark streaks often occur in the xylem tissue of infected roots and lower stem, and the roots may decay. Infected seedlings may wilt and die. This results in substantial loss of production.

Strains of Fusarium are usually host-specific and are designated according to their formae specialis (f. sp.). Plant pathogenic Fusarium species include F. graminearum, F. oxysporum and F. solani. F. graminearum commonly infects barley, wheat and maize, causing head blight as well as root rot and seedling blight. The total losses in the US of barley and wheat crops between 1991 and 1996 have been estimated at $3 billion. F. oxysporum f.sp. cubense causes Panama disease of banana (Musa secies.), also known as fusarium wilt of banana. Fusarium wilt of solanaceous plants is caused by the fungus F. oxysporum f. sp. lycopersici, which causes disease on tomato, eggplant and pepper plants. F. oxysporum f. sp. narcissi causes rotting of the bulbs and yellowing of the leaves of daffodils. In contrast to the plant-pathogenic Fusarium species, other Fusarium species have emerged as important opportunistic pathogens in humans causing hyalohyphomycosis, especially in burn victims and bone marrow transplant patients, mycotic keratitis and onychomycosis.

Pathogenic Fusarium fungi naturally invade the host plant from the soil via wounds or by penetrating the root tissue. Following entry, the fungus enters the vascular tissue, in particular the xylem, after which fungal conidia may spread throughout the host.

The present inventors designed and tested some constructs encoding modified RNA molecules for screening for reducing expression of fungal target RNA molecules and thereby reducing infection and disease caused by F. oxysporum, as follows. Two target genes were chosen for initial experiments to down-regulate gene transcripts in the fungus using RNA molecules of the invention. The first target gene selected was one encoding a 1,3 β-glucan synthase (Fks1), also known as a PT48 protein, which is responsible for the synthesis of one of the main components of the fungal wall, the polymer β-glucan (Harris and Stone, 2009). The nucleotide sequence of a Fks1 protein coding region of a cDNA from F. oxysporum and the encoded amino acid sequences are presented herein as SEQ ID NOs: 216 and 217. The nucleotide sequence of SEQ ID NO: 216 was 99.4% identical to the coding region of Accession No. XM_031173665.3 which encodes a homolog in F. oxysporum strain Fo47, which is an endophytic strain used as a biocontrol agent (Wang et al., 2020).

The second target gene selected encoded a F-box protein involved in pathogenicity from F. oxysporum f. sp. cubense (Frp1), Accession No. KJ025076.1. The corresponding Frp1 protein is required for pathogenicity of F. oxysporum f. sp. lycopersici on tomato (Duyvesteijn et al., 2005). The nucleotide sequence of a Frp1 protein coding region of a cDNA from F. oxysporum and the encoded amino acid sequence are presented herein as SEQ ID NOs: 218 and 219.

A nucleotide sequence from within each of the protein coding regions of the Fks1 and Frp1 cDNAs was selected as a target sequence to make the RNA molecules for down-regulating these genes simultaneously. One was a 350 nt sequence corresponding to nucleotides 4899-5248 of SEQ ID NO: 216, the other a 341 nt sequence corresponding to nucleotides 1090-1430 of SEQ ID NO: 218. These two sequences were joined as a single, chimeric sequence (SEQ ID NO: 220) which was used to make a genetic construct encoding a conventional, chimeric hairpin RNA. That hairpin RNA (hpFoFks1/Frp1[Con]) had fully canonical basepairing in a 701-basepair dsRNA region comprising 691-basepairs of the target-specific sequences. A construct encoding a second hairpin (hpFoFks1/Frp1[GU]) was designed having 194 C to T substitutions in the chimeric sense sequence to result in a modified hairpin RNA having G:U basepairs (27.7% G:U). The loop sequence in both cases was a chimeric antisense sequence, using a 140 nt sequence which was complementary to nucleotides 4758-4897 of SEQ ID NO: 216 joined to a 138 nt sequence which was complementary to nucleotides 1432-1569 of SEQ ID NO: 218, flanked by 10 nt sequences that formed part of the dsRNA region but not related to the target sequences. It was considered that the chimeric loop sequence, antisense to regions of both target RNA molecules, might enhance the effectiveness of the down-regulation relative to a corresponding hairpin RNA molecule with an unrelated sequence or an intron sequence. The arrangement of elements in the expression cassettes was: promoter-chimeric antisense sequence-chimeric antisense loop sequence-chimeric sense sequence-transcription terminator. The nucleotide sequences encoding the hairpin RNA molecules hpFoFks1/Frp1[Con] and hpFoFks1/Frp1[GU] including a T7 RNA polymerase promoter immediately upstream of the RNA coding region are provided herein as SEQ ID NOs: 221 and 222.

Two analogous constructs were made in the ledRNA format using the same sense and antisense sequences. The first dsRNA region targeted the Fks1 transcript while the second dsRNA region targeted the Frp1 transcript. The loops were also target-specific for the transcripts, having an antisense sequence of 140 nt as the loop sequence. The nucleotide sequences encoding the ledRNA molecules ledFoFks1/Frp1[Con] and ledFoFks1/Frp1[GU] including a T7 RNA polymerase promoter immediately upstream of the RNA coding region are provided herein as SEQ ID NOs: 223 and 224.

The four genetic constructs were made in an expression vector including a CaMV 35S promoter for expression in plant cells, and the expression cassette inserted into a binary vector pART727 for plant transformation. The four genetic constructs that were made were thereby:

Construct 30.1. Encoding conventional, symmetric hairpin with fully canonical basepairing targeting Fks1 and Frp1 (hpFoFks1/Frp1[Con]); SEQ ID NO:xxx.
Construct 30.2. As for Construct 30.1 except modified with 76 C to T substitutions in the chimeric sense sequence, targeting Fks1 and Frp1, SEQ ID NO:xxx (hpFoFks1/Frp1[GU]).
Construct 30.3. Encoding symmetric ledRNA with fully canonical basepairing targeting Fks1 and Frp1 (ledFoFks1/Frp1[Con]).
Construct 30.4. As for Construct 30.3 except modified with 76 C to T substitutions in the sense sequence, targeting Fks1 and Frp1, (ledFoFks1/Frp1[GU]).

The DNA sequences were synthesised by a commercial provider and the assembled DNA fragments inserted into a plant expression vector. The expression constructs were inserted into binary vector pART727. The transcribed regions including a T7 RNA polymerase promoter are inserted between an upstream CaMV 35S promoter and a downstream Ocs3′ transcription terminator. This expression system provides for strong, constitutive expression of the RNA molecules in the roots of plant cells. To produce the in vitro transcripts, each construct DNA was digested with restriction enzyme SpeI and used for transcription with T7 RNA polymerase as described in Example 1.

Agrobacterium cells were transformed using the binary constructs and transformed cells obtained. Confirmed Agrobacterium transformants were used to transform Arabidopsis thaliana plants of the Columbia-0 (Col-0) ecotype by the floral dip method, selecting cells that were transgenic for the T-DNAs through selection for the NptII marker gene, and at least 20 confirmed transgenic plants obtained for each construct. Northern blot analysis was used to confirm that the constructs were expressing the RNA molecules in the plant cells and to compare expression levels. T2 seed was obtained from T1 plants and confirmed to be homozygous for the transgene. The subsequent T2 plants were tested for increased resistance to F. oxysporum using the following methods.

### Plant Growth Conditions

The plants used in the first fungal resistance assay were T2 homozygous expressing either hpFoFks1/Frp1[Con], hpFoFks1/Frp1[GU], transformants ledFoFks1/Frp1[Con] or ledFoFks1/Frp1[GU] RNA molecules. The negative controls used in the experiment were transformants expressing hpRNA constructs targeting the β-glucuronidase (GUS) gene or wild type plants, treated in the same manner. Seeds from these plant lines were gas sterilised for two hours using 100 ml bleach and 3 mL 36% HCl. Approximately 100 sterilised seeds were grown on each plate containing ½ MS (Murashige and Skoog) medium with 1 mL/L kanamycin. Plated seeds were cold stratified at 4° C. in the dark for about 3 days before transfer to 16 h light/8 h dark conditions at 23° C. After 12 days, about 20 healthy plants with true leaves were transferred to fresh ½ MS plates.

Fusarium oxysporum Growth Conditions

F. oxysporum strain Fo5176 was obtained from Dr Roger Shivas, Queensland Plant Pathology Herbarium, Queensland Department of Primary Industries and Fisheries (QDPI&F), Brisbane, Australia (Thatcher et al., 2009). The fungus was grown in 50 mL half strength Potato Dextrose Broth (12 g PDB per L), shaking at 28° C. for three days. The F. oxysporum culture was filtered through Miracloth to isolate spores without hyphae and diluted 1 in 10 with water. The OD600 of the filtrate was measured and total spores calculated using the formula:

spores/mL=(10×OD600)×457445−11830

The filtrate was centrifuged and the supernatant discarded. The spores were resuspended to a concentration of 1×10⁶ spores per mL distilled water. The spore suspension was then transferred to a small petri dish.

The plant seedlings, grown as described above for about 2 weeks following seed germination, were inoculated with the F. oxysporum spore solution. The roots of 22-24 seedlings per line were dipped in the spore solution, being careful not to dip the leaves. Excess solution was let run off into the dish, and the seedlings placed onto fresh plates containing ½ MS media without sucrose to encourage fungal infection of plants. Plant seedlings from a second, control batch were dipped in water instead of fungal spores, to serve as a control. The seedlings were grown on ½ MS plates without sucrose for 2 days before being transferred to soil, 10 plants per pot for each line, using Debco potting mix with 2 g/L Osmocote mini.

### Results

The progression of fungal infection of plants was measured at 8- and 16-days post inoculation (dpi) by calculating the percentage of plants per line with yellowing symptoms in leaves or vasculature. The percentage measurements from each plant were determined and the mean of each two replicates, being two pots containing plants from the same line, calculated.

Northern blots were carried out to examine hpRNA accumulation, using the methods as described in Example 1. RNA was extracted from leaves or from the aerial parts from approximately seven T2 plants transformed with each construct. The radiolabelled probes for hybridisation targeting stem sequences of the hpRNA constructs were made following the New England Biolabs Standard RNA Synthesis protocol.

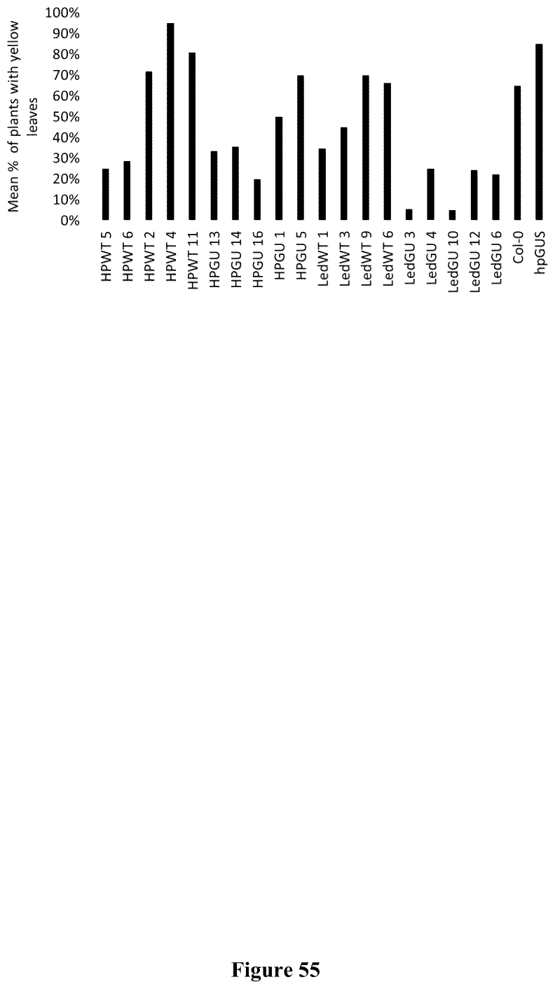

It was observed in a first inoculation experiment that approximately 70-100% of the wild-type A. thaliana plants and hpGUS control plants showed symptoms of infection, in particular showing an initial yellowing of leaves, then leaf necrosis. For most control plants, most of the leaves became necrotic, leading to seedling death by 16 days post-inoculation. In contrast, several transgenic lines transformed with Construct 30.1 showed less necrosis and less plant death than the control plants, in particular plants of hpFoFks1/Frp1[Con] lines 5, 6 and 12. This resistance phenotype was more pronounced for the plant lines expressing the hpFoFks1/Frp1[GU] RNA molecules from construct 30.2, in particular lines 12, 13 and 14. The plants expressing the ledFoFks1/Frp1 RNA molecules also showed increased fungal disease resistance, in particular lines 1 and 3 for the ledFoFks1/Frp1[Con] expressing plants, and lines 3, 4, 6, 10 and 12 for the ledFoFks1/Frp1[Con] expressing plants which were the most resistant. These results are presented graphically in FIG. 55.

The inoculation experiment was repeated and essentially the same observation was made, that the ledRNA molecules with the G:U modification in the dsRNA region provided the best level of resistance for the four constructs tested.

The Northern blot analysis showed that the plants that were the most resistant tended to be the highest expressors of the transgenes i.e. they accumulated more of the RNA molecules targeting the Fks1 and Frp1 RNA molecules. Probing for antisense sRNA molecules, it was observed that the hpFoFks1/Frp1[Con] plants accumulated the greatest amount of 21- and 24-mers. It was concluded that the conventional hairpin RNA were processed more efficiently by Dicers than the G:U modified molecules.

Similar constructs are made to express asymmetric RNA molecules having the Δ22 modification targeting the same chimeric target sequence, both in the hairpin and ledRNA formats, with or without the G:U modification. These constructs are used to transform A. thaliana plants and other plants such cotton and tomato.

### Example 31: Precursor RNA Molecules that Target Genes in Verticillium

Verticillium dahliae, a fungus in the Ascomycota, is a soil-borne fungal pathogen that causes a wilting disease, Verticillium wilt, of plants from more than 200 dicotyledonous plant species such as important crops, flowers, vegetables, trees and shrubs. These include cotton, Brassicaceae such as Brussels sprouts and cabbage (Brassica oleracea), solanaceous plants such as tomatoes, eggplant (Solanum melongena) and peppers (Capsicum spp.), potato (Solanum tuberosum), cucumber (Cucumis sativus), spinach, and Cucurbita species such as pumpkin, Cucurbitaceae plants such as watermelon, honeydew and cantaloupe.

V. dahliae invades the host plant via natural wounds or by penetrating the root tissue. Following entry, the pathogen enters the vascular tissue, in particular the xylem, after which fungal conidia may spread throughout the host. The plant responds to the pathogen by producing tyloses which block the xylem, resulting in decreased water flow to upper parts and wilting. Symptoms of this disease, aside from wilting, include leaves that have abnormal coloration or necrotic areas. The affected leaves may senesce and defoliate. The stem often has discolored vascular tissue and the plant may be stunted. V. dahliae can persist in the soil in the form of microsclerotia which may lie dormant for years, so controlling the pathogen by fallowing or crop rotation generally has little success.

Verticillium wilt has been reported to cause reduction in cotton yield in the range of 10-62%. In Australia, there are currently three identified strains of V. dahliae; two non-defoliating strains (VCGs 2A and 4B) and a defoliating strain (VCG 1A). The defoliating pathotype is deemed to be highly pathogenic, often inducing defoliation and sometimes lethal to cotton plants. In contrast, the non-defoliating pathotype is considered less aggressive and generally does not cause defoliation.

More than 40 different versions of the V. dahliae genome sequence have been published, with genome assembly sizes ranging from 31.97 to 40.17 Mbases, so making available gene sequences for this species. Numerous genes in V. dahlia have been reported to be important for infection or pathogenicity, including VdPKAC1, VMK1, VdMsb, VdGARP1, VDH1, Vayg1 and VGB which are involved in microsclerotia formation and the pathogenic process (Zhang et al., 2020b), VdSNF1 and VdSSP1 which are related to cell wall degradation, VdNEP, VdpevD1, VdNLP1 and VdNLP2 which encode effector proteins are involved in the pathogenic reaction, and VdFTF1, Vta2 and VdSge1 which encode transcriptional factors regulating pathogenicity genes. All of these genes can be targeted with the RNA molecules of the present invention.

Several genes were chosen for experiments to down-regulate gene transcripts in V. dahlia using RNA molecules of the invention. These included two different genes encoding leucine-rich repeat (LRR) proteins and single genes encoding a cyclopentanone 1,2-monooxygenase (CPM), a NADPH oxidase (NoxA), a thiamine thiazole synthase (TTS) and a thiamine transporter (TTR). The function of these genes in the pathogen are described as follows.

LRR proteins are characterised by repeated leucine-rich motifs. Many plant disease-resistance genes are in the LRR protein class, characterised by the presence of structural domains such as a nucleotide-binding site (NBS), a leucine-rich repeat (LRR), a Toll/Interleukin-1 receptor (TIR) domain or a coiled-coil (CC) domain, and in some a serine/threonine protein kinase domain (PK). These proteins and their domains are well known in the art. Some fungal LRR proteins have a role in pathogenesis as effectors that manipulate host plant processes to facilitate infection and promote disease development. LRR proteins can act as receptors or co-receptors that perceive signals, such as from the host plant, and trigger downstream responses critical for the pathogen's adaptation and survival. LRR proteins may also be involved in regulating immune signaling pathways within the pathogen, helping it to modulate its own immune responses and survive in the host plant.

Cyclopentanone 1,2-monooxygenase (CPM), encoded by the gene designated VDAG_03943 in V. dahliae, is expressed at a high level during the early stages of V. dahlia interaction with cotton plants and is crucial for the germination of microsclerotia (Hu et al., 2014; Luo et al., 2019). The microsclerotia are dormant spore-like structures that allow the fungus to survive adverse conditions and initiate infection when conditions are favourable. The CPM enzyme catalyses the oxidation of cyclopentanone, facilitating its breakdown as part of the metabolic processes necessary for microsclerotium germination. The ability to germinate from microsclerotia is a key factor in the pathogenicity of V. dahliae, enabling the fungus to transition from a dormant state to an active pathogenic state and successfully infect host plants. CPM genes involved in this process are therefore essential for the successful colonisation and infection of plants. A gene encoding CPM was identified by Zhang et al., (2020) from RNA-seq data as being upregulated upon treatment of V. dahlia microsclerotia with exudates from cotton roots at an early stage of interaction (6 h and 12 h). A deletion mutant in the VdCPM gene exhibited several developmental defects including slower colony growth, reduced germination rate of microsclerotia and decreased germ tube length compared to the wild-type strain. Moreover, the deletion strain lost its pathogenicity to cotton, suggesting that the CPM protein may have other functions related to pathogenicity.

NADPH oxidase (NoxA) is a membrane-bound enzyme complex that produces reactive oxygen species (ROS) by reducing molecular oxygen using NADPH as an electron donor. This enzyme is crucial for maintaining redox homeostasis and essential for the development, pathogenicity and stress tolerance of V. dahliae. Deletion of the noxA gene significantly impairs the disease by disrupting the formation of infectious structures like hyphopodia and penetration pegs which are vital for root penetration and host colonization. NoxA is essential for normal fungal development, with knockout mutants showing impaired growth, reduced conidiation, defective microsclerotia formation, thinner hyphae and compromised aerial mycelium production. Additionally, NoxA plays a vital role in managing oxidative stress, with mutants exhibiting high sensitivity to oxidative agents and impaired regulation of antioxidant genes.

The enzyme thiamine thiazole synthase (TTS) plays a key role in the fungus in the biosynthesis of the thiamine precursor thiazole. TTS catalyzes the conversion of nicotinamide adenine dinucleotide (NAD) and glycine into adenosine diphosphate 5-(2-hydroxyethyl)-4-methylthiazole, an intermediate in the thiamine (vitamin B1) biosynthesis pathway. Thiamine is essential for various cellular processes, including energy metabolism and the synthesis of nucleic acids. The production of thiazole, therefore, is needed for thiamine biosynthesis, supporting the growth, development and pathogenicity of V. dahlia (Hoppenau et al., 2014).

The thiamine transporter (TTR) of V. dahliae is also involved in its pathogenesis, needed for importing thiamine into the fungal cell. Thiamine (vitamin B1) is essential for fungal growth, invasion and reproduction, and can be synthesized de novo or acquired from the host plant. Recent research indicated that knockout mutants of the TTR gene showed significantly reduced virulence (Wang et al., 2024).

Other genes were also identified as candidates for targeting by the RNA molecules of the present invention. These include genes encoding a sugar transporter (VDAG_03649) since sugar metabolism plays an important role in the growth and pathogenicity of V. dahlia, a hexose transporter (VDAG_09835) which mediates transport of sugar across the cell membrane, a NAD/NADP transhydrogenase (VDAG_09269) which has an important role in cellular redox homeostasis by catalysing the transfer of hydride ions between NADH and NADP⁺, an amidase (VDAG_09707) which contributes to the pathogen's ability to infect hosts by degrading plant tissues and assimilating nitrogen efficiently, a transcription elongation factor SPT5 (VDAG_01211), a mitochondrial chaperone BCS1 which is involved in chaperone-mediated protein complex assembly, mitochondrial respiratory chain complex III assembly and protein insertion into mitochondrial inner membrane from matrix, a NADH dependent D-xylose reductase which is involved in xylose catabolisman ankyrin repeat protein that may facilitate protein-protein interactions and be involved in various cellular processes, including signaling, cell cycle control, and the maintenance of cellular structure, a cyanide hydratase (VDAG_08712) that catalyses the conversion of cyanide, a potent inhibitor of cellular respiration found in many plants as defence compounds, into the less toxic formamide, a Sge1 transcription factor that is a global regulator of pathogenicity, also required for radial growth and production of asexual conidiospores, a sucrose non-fermenting protein kinase (VdSNF1) that regulates the activity of pectinase and galactase and is required for virulence, nuclear transcription factors Som1 and Vta3 that support fungal adhesion and root penetration, septa positioning and the size of vacuoles, hyphal development, and regulate developmental genetic networks required for conidiation, microsclerotia formation and pathogenicity of V. dahlia, a cytochrome P450 monooxygenase (VdCYP1), an effector VdSC1 that contributes to virulence, and pathogenicity-related gene VdPR1. Others include a pantothenate transporter (VDAG_02269) which might be involved in vitamin B5 transport, essential for fungal metabolism, two endo-1,4-beta-xylanases (VDAG_03790, VDAG_06165) which are involved in breaking down plant cell walls, facilitating infection, and an xylosidase/arabinosidase (VDAG_01866) which contributes to the breakdown of complex sugars. For some of these genes, previous knockout studies in V. dahlia or other fungal pathogens have demonstrated the role of the gene products in virulence or pathogenicity.

The nucleotide sequences for cDNAs for the six selected target genes were obtained from a V. dahliae genome sequence. These nucleotide sequences and the amino acid sequences of the encoded proteins are presented herein as SEQ ID NOs: 225 to 236. From each of the sequences, a 300 nt sequence was selected as the target RNA sequence from within the protein coding region of the cDNA, as detailed in the legends to the SEQ ID NOs. Multiple constructs were then made for each of the target RNA transcripts using a conventional hairpin structure with an unmodified sense sequence as the control for the experiment, one encoding a hairpin RNA with C nucleotides in the DNA sense sequence substituted with T nucleotides to provide a hpRNA[G:U] structure, another encoding a hairpin RNA with A nucleotides in the DNA sense sequence substituted with G nucleotides to provide an alternative hpRNA[G:U] structure, one encoding a hairpin RNA with the Δ22 modification hpRNA[Δ22] and in some cases, one encoding a ledRNA structure. Combinations of the modifications were also conceived. For each structure, a 130 nt sequence from a GUS cDNA, corresponding to nucleotides 805-934 of SEQ ID NO: 1 and therefore not related to the target transcript, was selected as the loop sequence. When these DNA constructs were transcribed, the resultant RNA molecules did not have an intron in them. Each DNA construct also had a T7 RNA polymerase promoter immediately upstream of the RNA coding sequence and a SpeI restriction enzyme site downstream to provide for in vitro transcription. The arrangement of elements was therefore: promoter-sense sequence-loop sequence-antisense sequence-terminator. The DNA sequences of the regions encoding the RNA molecules are provided herein as SEQ ID NOs: 237 to 256.

The sense sequence of the construct encoding the first G:U hairpin RNA molecule (SEQ ID NO: 238) targeting LRR1 (SEQ ID NO: 238) was produced by replacing 76 C nucleotides with T nucleotides, resulting in a hairpin RNA with 25.3% of the basepairs in the dsRNA region being G:U basepairs. The longest stretch of contiguous canonical basepairs in the dsRNA region was 11 contiguous basepairs. The sense sequence of the construct encoding the second G:U hairpin RNA molecule (SEQ ID NO: 239) targeting LRR1 was produced by replacing 73 A nucleotides with G nucleotides, resulting in a hairpin RNA with 24.3% of the basepairs in the dsRNA region being G:U basepairs. The longest stretch of contiguous canonical basepairs in the dsRNA region was 16 contiguous basepairs. The sense sequence of the construct encoding the Δ22 hairpin RNA molecule (SEQ ID NO: 240) targeting LRR1 was produced by deleting the 10th nucleotide of the sense sequence and then every 22^{nd} nucleotide, relative to the unmodified sense sequence, a total of 14 nucleotides deleted. This resulted in a modified sense sequence of 286 nucleotides compared to the 300 nucleotides of antisense sequence i.e. 95.3% of the length relative to the antisense sequence. The construct encoding the ledRNA molecule (SEQ ID NO: 241) targeting LRR1 was made by splitting the sense sequence into two parts, each of 150 nucleotides, and arranging the elements as in FIG. 52B. The second loop sequence had 130 ribonucleotides corresponding to nucleotides 935-1064 of SEQ ID NO: 1. The other genetic constructs had analogous designs except targeting 300 nt regions of different target RNA transcripts.

The resultant genetic constructs were:
Construct 31.1. Encoding conventional, symmetric hairpin with fully canonical basepairing targeting LLR1 (hpVdLRR1[Con]); SEQ ID NO: 237.
Construct 31.2. As for Construct 31.1 except modified with 76 C to T substitutions in the sense sequence, targeting LLR1, SEQ ID NO: 238 (hpVdLLR1[GU]1).
Construct 31.3. As for Construct 31.1 except modified with 73 A to G substitutions in the sense sequence, targeting LLR1, SEQ ID NO: 239 (hpVdLLR1[GU]2).
Construct 31.4. Encoding an asymmetric hairpin as for Construct 31.1 except every 22nd nucleotide deleted from the chimeric sense sequence, all basepairs being canonical basepairs, targeting LLR1, SEQ ID NO: 240 (hpVdLLR1[Δ22]).
Construct 31.5. LedRNA molecule with only canonical basepairing, targeting LLR1, SEQ ID NO: 241 (ledVdLRR1[Con]).
Construct 31.6. Encoding conventional, symmetric hairpin with fully canonical basepairing targeting LLR2 (hpVdLRR2[Con]); SEQ ID NO: 242.
Construct 31.7. As for Construct 31.6 except modified with 77 C to T substitutions in the sense sequence, targeting LLR2, SEQ ID NO: 243 (hpVdLLR2[GU]).
Construct 31.8. Encoding an asymmetric hairpin as for Construct 31.6 except every 22nd nucleotide deleted from the chimeric sense sequence, all basepairs being canonical basepairs, targeting LRR2, SEQ ID NO: 244 (hpVdLRR2[Δ22]).
Construct 31.9. Encoding conventional, symmetric hairpin with fully canonical basepairing targeting CPM (hpVdCPM[Con]); SEQ ID NO: 245.
Construct 31.10. As for Construct 31.9 except modified with 73 C to T substitutions in the sense sequence, targeting CPM, SEQ ID NO: 246 (hpVdCPM[GU]).
Construct 31.11. Encoding an asymmetric hairpin as for Construct 31.9 except every 22nd nucleotide deleted from the chimeric sense sequence, all basepairs being canonical basepairs, targeting CPM, SEQ ID NO: 247 (hpVdCPM[Δ22]).
Construct 31.12. Encoding conventional, symmetric hairpin with fully canonical basepairing targeting Nox (hpVdNox[Con]); SEQ ID NO: 248.
Construct 31.13. As for Construct 31.12 except modified with 73 C to T substitutions in the sense sequence, targeting Nox, SEQ ID NO: 249 (hpVdNox[GU]).
Construct 31.14. Encoding an asymmetric hairpin as for Construct 31.12 except every 22nd nucleotide deleted from the chimeric sense sequence, all basepairs being canonical basepairs, targeting Nox, SEQ ID NO: 250 (hpVdNox[Δ22]).
Construct 31.15. Encoding conventional, symmetric hairpin with fully canonical basepairing targeting TTR (hpVdTTR[Con]); SEQ ID NO: 251.
Construct 31.16. As for Construct 31.15 except modified with 73 C to T substitutions in the sense sequence, targeting TTR, SEQ ID NO: 252 (hpVdTTR[GU]).
Construct 31.17. Encoding an asymmetric hairpin as for Construct 31.15 except every 22nd nucleotide deleted from the chimeric sense sequence, all basepairs being canonical basepairs, targeting TTR, SEQ ID NO: 253 (hpVdTTR[Δ22]).
Construct 31.18. Encoding conventional, symmetric hairpin with fully canonical basepairing targeting TTS (hpVdTTS[Con]); SEQ ID NO: 254.
Construct 31.19. As for Construct 31.18 except modified with 107 C to T substitutions in the sense sequence, targeting TTS, SEQ ID NO: 255 (hpVdTTS[GU]).
Construct 31.20. Encoding an asymmetric hairpin as for Construct 31.18 except every 22nd nucleotide deleted from the chimeric sense sequence, all basepairs being canonical basepairs, targeting TTS, SEQ ID NO: 256 (hpVdTTS[Δ22]).

The genetic sequences were synthesised by a commercial provider and the assembled DNA fragments inserted into a plant expression vector. The transcribed regions including the T7 RNA polymerase promoter were thereby inserted between an upstream CaMV 35S promoter and a downstream Ocs3′ transcription terminator. This expression system provided for strong, constitutive expression of the RNA molecules in the roots of plant cells. To produce the in vitro transcripts, each construct DNA was digested with restriction enzyme SpeI and used for transcription with T7 RNA polymerase as described in Example 1.

To screen for reduction of target gene expression and to identify susceptible target genes, the RNA preparations are applied to V. dahliae conidia or microsporangia topically in in vitro fungal growth assays or applied to plant roots by soaking with subsequent application of the fungal conidia. Germination of the microsporangia and growth of hypha are measured, and the amount of each target RNA molecule is determined by qRT-PCR and Northern blotting analysis to detect reduction of target gene expression.

Agrobacterium cells were transformed with the plasmid constructs and transformed cells were selected. Confirmed transformants were used to introduce the T-DNAs into Nicotiana benthamiana plant leaves for transient expression, and transgenic N. benthamiana plants were generated expressing the constructs. Northern blot analysis is used to confirm that the constructs express the RNA molecules in the plant cells and to compare expression levels. The constructs are also transformed into Agrobacterium rhizogenes strains and used to prepare transgenic hairy root cultures of N. benthamiana and cotton plants. The roots are infected with V. dahliae and the penetration of the roots by the fungus assayed, as well as the morphology of the treated fungal hyphae. The amount of target RNA transcripts is measured by qRT-PCR. The production of increased levels of antisense 22-mers is observed for the constructs expressing the asymmetric RNA molecules. The constructs that show the greatest effect on the fungal pathogen are used to generate transgenic cotton plants that express the selected inhibitory RNA molecules.

### Example 32: Design of RNA Molecules for Reducing Expression of Genes in Varroa Mite

### Introduction

Varroa mite (Varroa destructor) is a serious parasite of the honeybee (Apis mellifera), feeding on bee larvae during pupal development inside capped cells in the beehive. Varroa mite also acts as a vector of several viral pathogens, in particular the widespread deformed wing virus (DWV), a mutualistic symbiont of the mite (di Prisco et al., 2016) which damages bee colonies. Conventional RNAi has been shown to be effective in reducing gene expression in the Varroa mite, for example as reported by Becchimanzi et al., (2024), Becchimanzi et al., (2020), Campbell et al., (2016), reviewed by Niu et al., (2018). Garbian et al., (2012) showed that dsRNA ingested by bees was transferred to the Varroa mite and from mites to a parasitized bee. Huang et al., (2015) targeted six candidate genes with dsRNA molecules, namely genes encoding the polypeptides Da, Pros26S, L8, L11, P0 and S13. Hunter et al., (2010) targeted a virus, Israeli Acute Paralysis Virus (IAPV) that infects honeybees in a trial simulating real field conditions. The dsRNA was shown to reduce bee mortality and improve the overall health of bees infected with IAPV. Leonard et al., (2020) used a symbiotic bacterium found in bee guts to express and deliver dsRNA to the insects—these could deliver dsRNA molecules targeting the Varroa mite. McGruddy et al., (2024) tested a dsRNA agent referred to as Vadescana (GreenLight Biosciences, Inc.) to assess the impact on Varroa destructor fitness following exposure to the dsRNA in the brood cells of the hive. Vadescana was designed to specifically target a gene encoding a calmodulin-like protein, which plays an important role in calcium regulation in the mite. The agent was reported to substantially reduce mite fertility.

Design of RNA Molecules with Variations in Structures

Three target genes in Varroa destructor were selected, based on the published reports mentioned above that showed an effect of conventional RNAi targeting those genes. These were: genes encoding a chitin-binding domain protein (Accession No. XP_022647894) which is somewhat similar to perotrophin-A, a crustacean hyperglycaemic hormone (CHH) (Accession No. XM_022802805.1, LOC111249228) and a calmodulin-like gene (Accession No. XM_022799184, LOC111247793), see also U.S. Pat. No. 10,927,374 B2. The nucleotide sequences of the cDNAs corresponding to these genes are provided herein as SEQ ID NOs: 257-259 and the amino acid sequences of the encoded polypeptides as SEQ ID NOs: 260-262. The chitin-binding protein is a short protein of 111aa (SEQ ID NO: 260) with a type-2 chitin-binding domain, characterised by a six-cysteine motif and several aromatic residues. Campbell et al., (2016) identified a crustacean hyperglycemic hormone/molt-inhibiting hormone/gonad-inhibiting hormone (CHH/MIH/GIH) preprotein of 134aa containing the motif Pfam PF011147 at positions 54 to 129, with six conserved cysteines.

A target region was selected within each cDNA sequence corresponding to the three genes. The sequences of the target regions are provided herein as SEQ ID NOs: 263-265. Using these sequences, four sets each of three genetic constructs were designed to encode (i) ledRNA molecules with sense sequences corresponding to the wild-type target regions, having only canonical basepairing in the dsRNA regions: ledCHIBIN[Con], ledCHH[Con] and ledCALL[Con], (ii) the sense sequences modified with all C nucleotides replaced with T nucleotides to provide for G:U basepairing in both dsRNA regions: ledCHIBIN[G:U], ledCHH[G:U] and ledCALL[G:U], (iii) ledRNA molecules having the Δ22 modification in the sense sequences by deleting every 22nd nucleotide, providing bulges in the antisense sequences: ledCHIBIN[Δ22], ledCHH[Δ22] and ledCALL[Δ22], or (iv) the sense sequences having the combination of the C to T substitutions with the Δ22 modification: ledCHIBIN[Δ22G:U], ledCHH[Δ22G:U] and ledCALL[Δ22G:U]. The nucleotide sequences encoding the symmetric RNA molecules for the chitin binding and CHH transcripts are provided as SEQ ID NOs: 266-269. The nucleotide sequences encoding the four constructs for the calmodulin-like transcript are provided as SEQ ID NOs: 270-273. Each of the RNA coding regions was placed under the control of a T7 RNA polymerase promoter for in vitro transcription.

Constructs were also designed encoding asymmetric ledRNA molecules having the Δ22 modification but having loop sequences which were sense sequences from the target transcripts, either upstream or downstream of the target region. These loop sequences are intended to promote initiation of more secondary sRNA molecules against the target transcript. One of these molecules has a combination of C to T and A to G substitutions in the sense sequences to provide a more even distribution of G:U basepairs in the dsRNA regions, so that about 20% of the basepairs are G:U basepairs while the other 80% are canonical basepairs. These RNA molecules are produced by in vitro transcription from an upstream T7 RNA polymerase promoter.

Varroa mites are soaked in solutions containing the RNA molecules, singly or in combinations of two or three of the RNA molecules, and reduction in gene expression is observed by qPCR. Experiments are also carried out with varroa-infested nurse bees in cages, feeding them a sugar solution with the RNA molecules, and counting how many survive over time. Reduction in gene expression in the mites is also observed by qPCR. The in vitro-transcribed RNA preparations are also formulated as bait and supplied to Varroa mites essentially as described by McGruddy et al., (2024). The number of progeny mites per capped cell in the treated beehives are assessed and compared to the number when Vadescana is used.

### Example 33: Design of RNA Molecules for Use as Herbicides

Weeds can be treated with the RNA molecules of the present invention, for example by applying RNA molecules topically that target genes in the plants for silencing in order to interfere with their growth, reproduction or other functions. That is, functioning as an herbicide. The use of RNA molecules as potential herbicides has been reviewed by Westwood et al., (2018) and Zabala-Pardo et al., (2022). The species Raphanus raphanistrum (wild radish) is a significant weed in Australia and around the world, producing abundant seeds and having the potential to interfere with crop harvesting and causing yield losses. Wild radish is competitive with crop plants because its seedlings establish rapidly and grow relatively quickly. This species was therefore used in tests of RNA molecules, as follows

Several target genes in R. raphanistrum were selected from a genomic sequence of the species. These were genes encoding the enzymes acetolactate synthase (ALS), two subunits of a 15-cis-phytoene desaturase (PDS1 and PDS2), a 5-enolpyruvylshikimate-3-phosphate synthase (EPSPS) and a family of genes encoding ATP-dependent zinc metalloproteases, namely filamentation temperature-sensitive H (FTSH) polypeptides. ALS catalyses the first, essential step in the synthesis of the branched-chain amino acids valine, leucine and isoleucine and so has been a target for herbicides. ALS inhibiting herbicides such as the imidazolinones, sulfonylureas, sulfonylamino carbonyltriazolinones and triazolopyrimidines have a broad spectrum of selectivity with regard to plant species. PDS is an essential plant enzyme for synthesizing carotenoids and a target of certain inhibitors such as norflurazon which act as bleaching herbicides. Homologs of these genes were identified in R. sativus (common radish), a closely related species to R. raphanistrum, as follows: chloroplastic ALS small subunit 2 (Accession No. XM_018612381), PDS1 (Accession No. XM_018631747), PDS2 (Accession No. XM_056993225), EPSPS (Accession No. XM_018628389), and a gene family encoding a filamentation temperature-sensitive H polypeptides (Accession Nos. XM_018589767, XM_018584578 etc). The corresponding R. raphanistrum sequences of the cDNAs for those genes were identified from a genomic sequence and some are provided herein as SEQ ID NOs: 274-278. The corresponding amino acid sequences of the encoded polypeptides are provided as SEQ ID NOs: 279-283.

A target region was selected within each cDNA sequence for the target genes. The sequence of the EPSPS target region is provided herein as SEQ ID NO: 284. Using these sequences, genetic constructs were designed to encode (i) dsRNA molecules having only canonical basepairing and without loop sequences, formed from sense and antisense RNA strands that annealed to form the dsRNA, where the sense sequence corresponded to the wild-type target region, and (ii) ledRNA molecules for the same target sequences but having the Δ22 modification by deleting every 22nd nucleotide and closed at each end by loop sequences. The loop sequences in the ledRNA molecules, for example the RNA molecule ledRrEPSPS[Δ22] encoded by SEQ ID NO: 285, were sense sequences from the target transcript outside of the target region, which therefore formed single-stranded loops which did not have a complementary sequence in the RNA molecules. Such additional sense sequences in the loops were considered to enhance the silencing effect. Each of the RNA coding regions was placed under the control of a T7 RNA polymerase promoter for in vitro transcription.

Genetic constructs encoding the RNA molecules were made in a vector such as pUC19. The plasmid DNAs were linearised with a blunt end-forming restriction enzyme and transcribed in vitro using a HiScribe T7 RNA Synthesis kit (New England Biolabs) according to the manufacturer's instructions with 1.2 μg template DNA per reaction. Each dsRNA product was precipitated with 0.1 vol 3 M sodium acetate and 1 vol 100% isopropanol and cooled overnight at −80° C. The precipitated RNA was collected by centrifugation and washed twice with 75% ethanol at 4° C., dried and resuspended in nuclease-free water.

For tests on excised leaves, fully expanded leaves were excised from young (2-3 weeks) wild radish plants grown in a glasshouse at 28° C., 60% relative humidity (RH) and under ambient lighting. The excised leaves were immediately transferred to 5 mL vials with the petiole immersed in 0.1 mM phosphate buffered saline solution. Leaves were sprayed with 50 μL of a solution containing 2 mg/mL RNA molecules dsRrEPSPS or ledRrEPSPS[Δ22], or dsGFP or water as negative controls, using a handheld atomizer. The surfactant Silwet L-77 was added to all treatments to a final concentration of 0.01% (v/v) immediately before spraying to enable even distribution of the spray across the leaf surface and possibly aiding penetration of the RNA molecules through the cuticular surface. The detached leaves in the vials were maintained in a growth chamber at 26° C., 75% RH with a 12 h day:light cycle. Five replicate leaves were used for each treatment.

In a second, parallel experiment, a combination of 0.01% (v/v) Silwet L77 and 0.5% Collide (Silwet+Collide) as surfactants was compared with Silwet L-77 alone to see if that increased the uptake of dsRNA. These agents were added immediately prior to spraying. Three replicates were used to test Silwet and Silwet+Collide in this experiment.

Leaves from the assays were sampled for gene expression 6 hours post treatment. Five or six leaf samples were taken from each leaf and total leaf RNA immediately extracted using an RNeasy Plant Kit (Qiagen). The extracts were treated with 1 U Turbo DNase (ThermoFisher Scientific) per μg of RNA to remove DNA, and the RNA purified by sodium acetate/isopropanol precipitation and 75% ethanol wash. 50 ng of purified leaf RNA was amplified by qRT-PCR in triplicate using the SensiFAST SYBR One-Step qPCR kit (Millenium Sciences) following the manufacturer's instructions using oligonucleotide primers specific for the target transcript (RrEPSPS) or a reference gene (RrGAPDH). Changes in the expression was calculated as the fold-change in relative expression (2-ΔΔCT) compared to the mock treatment, averaged across each treatment group.

A decrease was observed in EPSPS mRNA levels in the leaves for all the treatments including the GFP control when compared to the control leaves sprayed with water, indicating some non-specific effect was occurring. However, the asymmetric RNA molecule ledRrEPSPS[Δ22] induced significantly stronger silencing of the EPSPS gene compared to the conventional double-stranded dsRrEPSPS 6 hours after application. Similar results were achieved when a combination of two agents Silwet+Collide was added to the ledRrEPSPS[Δ22] RNA, with significantly greater reduction of gene expression by the asymmetric RNA. The efficacy may be further improved by optimising the formulation with different adjuvant concentrations, pH and other additives.

In a further experiment, seeds of R. raphanistrum were sown in a tray and the seedlings maintained in a glasshouse at 28° C., 60% RH and ambient lighting. Each tray contained about 20 plants and a total of four trays were used for this experiment. When plants were 7 to 10 days old with 3 to 4 fully expanded true leaves, each tray was sprayed with a solution containing 6 mg/mL dsRrEPSPS, or ledRrEPSPS[Δ22] to all adaxial leaf surfaces using an atomizer. Silwet L-77 was added to all treatments to a concentration of 0.01% (v/v) immediately prior to spraying. Changes in the plant phenotype were monitored after the treatments. Patches of necrotic lesions were observed in plants treated with the dsRrEPSPS and ledRrEPSPS[Δ22] molecules 48 hours after application, with more damage observed in the plants treated with ledRrEPSPS[Δ22].

To assess the presence and integrity of the RNA molecules after application to the plants, leaves from four plants per tray were excised 7 days post-treatment and thoroughly washed with 500 μL nuclease-free water to wash off any adhering RNA. RNA was then precipitated from the wash solution using 0.1 volume of 3 M sodium acetate and 1 volume of isopropanol and cooled overnight at −80° C. The precipitated material was collected by centrifugation and resuspended in 0.06 volume nuclease-free water. The concentration of RNA was measured by spectrophotometry and approximately 5 μg was analysed by gel electrophoresis on agarose gels. Some degradation of the dsRrEPSPS molecules was evident as seen from smearing of the RNA in gel, whereas the RNA band for ledRrEPSPS[Δ22] closely resembled that of the purified RNA sample used in the treatments. It was evident that a substantial amount of RNA remained on the leaf surface 7 days post-treatment. Optimising the formulation may allow for better uptake by plant cells and enable improved effect.

It was concluded in these initial experiments that the asymmetric RNA molecule yielded greater gene silencing and stability than the canonically basepaired dsRNA molecule without loops, but improvements to the formulation and delivery were desired.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from AU 2023904203 filed 22 Dec. 2023, AU 2024900066 filed 10 Jan. 2024, AU 2024901860 filed 18 Jun. 2024, AU 2024902006 filed 28 Jun. 2024, AU 2024902008 filed 28 Jun. 2024, and AU 2024902009, filed 28 Jun. 2024, the entire contents of all of which is incorporated herein by reference.

All publications discussed and/or referenced herein are incorporated herein in their entirety.

The application contains a sequence listing in computer readable form. The file is hereby incorporated by reference into the instant disclosure.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abbasi et al. (2020). Insect Biochemistry and Molecular Biology 127 (2020) 103492.
Abd El Halim et al. (2016). Scientific Reports 6:29301 doi: 10.1038/srep29301.
Abo Al Ela et al. (2021). J. Agric. Food Chem. 2021:4343-4355.
Adeyinka et al. (2020). Molecular Biol Rep. 47:6309-6319.
Ai et al. (2019). Bulletin of Insectology 72:115-124.
Ajjappala et al. (2014). Planta 241:773-787.
Al Baki et al. (2020). Insects 2020, 11, 211; doi: 10.3390/insects11040211.
Alam et al. (2023). Viruses 2023, 15, 2050. doi.org/10.3390/v15102050.
Allan et al. (2005). Physiol Genomics 22:128-138.
Allen et al. (2005). Cell, 121:207-221.
Allen et al. (2021). Front. Agron. 3:591539. doi: 10.3389/fagro.2021.591539.
Allen et al. (2012). Journal of Insect Physiology 58:391-396.
Alvarez et al. (2000). Theor Appl Genet 100:319-327.
Alves et al. (2010). Journal of Insect Science: Vol. 10 Article 162.
An et al. (2023). Pest Manag Sci 79:2934-2943.
Annis et al. (1981). Environ Entomol 10:307-312.
Apirajkamol et al. (2020). Ecology and Evolution. 10:5680-5693.
Apirajkamol et al. (2023). Arch Microbiol, 205:328 doi.org/10.1007/s00203-023-03669-8.
Apone et al. (2014). Journal of Insect Science: 14 Article 87.
Arias et al. (2015). J. Vis. Exp. (106), e53398, doi: 10.3791/53398 (2015).
Arimatsu et al. (2007). J. Insect Biotechnol Sericology 76:57-62. doi: 10.11416/jibs.76.1_57.
Arjunan et al. (2024). Mol Biol Rep 51:355 doi.org/10.1007/s11033-023-09187-6.
Asokan et al. (2013). Bulletin of Entomol Res. 103:555-563.
Asokan et al. (2014). J Pest Sci. 87:163-172.
Atkinson et al. (2012). Current Opinion in Biotechnology 23:251-256.
Attasart et al. (2009). Virus Research 145:127-133.
Attasart et al. (2013). Virus Research 171:252-256.
Attia et al. (2022). Journal of Plant Diseases and Protection 129:1025-1030.
Azuma et al. (1995). FEBS Lett. 361:153-156.
Bai et al. (2023). Journal of Pest Science 96:1667-1681.
Bakhetia et al. (2005). Mol Plant Microbe Interact 18:1099-1106.
Bally et al. (2016). Front Plant Sci. 7:1453.
Bally et al., (2018). Pest Manag Sci 74:1751-1758.
Baltes et al. (2014). The Plant Cell 26:151-163. doi: 10.1105/tpc.113.119792.
Banerjee et al. (2017). Front. Plant Sci. 8:834. doi: 10.3389/fpls.2017.00834.
Banerjee et al. (2018). Front. Plant Sci. 8:2266. doi: 10.3389/fpls.2017.02266.
Baniya et al. (2021). Eur J Plant Pathol 161:439-452.
Baum et al. (2007). Nature Biotechnol. 25:1322-1326.
Baum and Roberts (2014). Advances in Insect Physiology, 47:249-295.
Baumlein et al. (1991). Mol. Gen. Genet. 225:459-467.
Baumlein et al. (1992). Plant J. 2:233-239.
Bautista et al. (2009). Insect Biochemistry and Molecular Biology 39:38-46.
Becchimanzi et al. (2020). PLoS Pathog 16 (12): e1009075.
Becchimanzi et al. (2024). Insect Mol Biol. 2024; 1-12.
Beck et al. (2009). FEBS Lett 583:2701-2709.
Beller et al. (2008). PLOS Biol. doi.org/10.1371/journal.pbio.00602 92.
Belles (2010). Annu. Rev. Entomol. 55:111-28.
Bender et al. (1997). Cell 91:777-788.
Bensoussan et al., (2020). Scientific Reports 10:19126 doi.org/10.1038/s41598-020-75682-6.
Bensoussan et al. (2022). Scientific Reports 12:14791 doi.org/10.1038/s41598-022-19231-3.
Bharathi et al. (2023). Plant Physiology and Biochemistry 194:600-618.
Bharti et al. (2017). Mol Biotechnol 59:343-352.
Bhatia and Bhattacharya (2017). Pest Manag Sci 74:2059-2068.
Bhatia et al. (2012). PLoS ONE 7 (10): e46343. Doi: 10.1371/journal.pone.0046343.
Bhattacharya et al. (1990) Cell 60:155-122.
Bicker et al. (2004). Cell and Tissue Research 317:207-220.
Blevins et al. (2006). Nucleic Acids Research 34:6233-6246.
Bober and Rafaeli (2010). Proc Natl Acad Sci USA. 107:16858-16862.
Bodemann et al. (2012). Proc. R. Soc. B 279:4126-4134.
Bolognesi et al. (2012). PLOS One 7: e47534.
Bouche et al. (2006). EMBO Journal, 25:3347-3356.
Bragg and Rieske (2022a). Front. Plant Sci. 2022, 13, 830226.
Bragg and Rieske (2022b). Int. J. Mol. Sci. 2022, 23, 9167.
Brant and Budak (2018). Front Plant Sci, 9, 1038.
Brar et al. (1996) Biotech Genet. Eng Rev 13:167-79.
Brizzee et al. (2023). Fermentation 2023, 9, 999. doi.org/10.3390/fermentation9120999.
Broothaerts et al. (2005). Nature 433:629-633.
Broun et al. (1998). Plant J. 13:201-210.
Buchanan-Wollaston, (1994) Plant physiology 105:839-846.
Burand and Hunter, (2013). J. Invertebrate Pathology 112: S68-S74.
Busk et al. (1997). Plant J. 11:1285-1295.
Cai et al. (2018). Science 360:1126-1129.
Cai et al. (2019). PLoS Pathogens 15 (12): e1008090.
Calderon-Urrea et al., (2012). Plant Biotechnol Rep 6:263-274.
Campbell et al. (2016). Insect Biochemistry and Molecular Biology 70:116.
Cao et al. (2024). Physiological and Molecular Plant Pathology 129 (2024) 102182.
Cappelle et al. (2016). Insect Molecular Biology doi: 10.1111/imb.12222.
Carney et al. (1997). Proc Natl Acad Sci USA. 94:12024-12029.
Chaitanya et al. (2017). Journal of Asia-Pacific Entomology 20:637-643.
Chandra et al. (2018). Appl Biochem Biotechnol. 184:746-761.
Chandra et al. (2018b). Pest Manag Sci 75:506-514.
Charlton et al. (2010). International Journal for Parasitology 40:855-864.
Chaudhary et al. (2019). Transgenic Res 28:327-340.
Chauhan and Rajam (2024). Planta (2024) 259:79, doi.org/10.1007/s00425-024-04360-y.
Chauveau et al. (2014). Human Mutation 35:1046-1059.
Chen et al. (2005). Functional Plant Biology 32:671-681.
Chen et al. (2005b). MPMI 18:621-625.
Chen et al. (2009). Mol Plant. 2:738-754.
Chen et al. (2008). Cell Tissue Res 331:725-738.
Chen et al. (2010). Proc Nat Acad Sci U.S.A. 107:15269-15274.
Chen et al. (2012). BMC Molecular Biol 13:31.
Chen et al. (2015). Ecotoxicology 24:595-603.
Chen et al. (2016). Journal of Experimental Botany 67:4979-4991.
Chen et al. (2021). Insects 2021, 12, 712. doi.org/10.3390/insects12080712.
Chen et al. (2022). Plant Biotechnology Journal 20:253-255.
Chen et al. (2023). Current Opinion in Plant Biology 2023, 76:102441.
Chen et al. (2023b). Front. Plant Sci. 14:1123523. doi: 10.3389/fpls.2023.1123523.
Cheng et al. (2015). Scientific Reports 5:9245 doi: 10.1038/srep09245.
Chikate et al. (2016). Pesticide Biochem Physiol. 133:44-51.
Chikwamba et al. (2003). Proc. Natl. Acad. Sci. U.S.A. 100:11127-11132.
Chimwai et al. (2016). Journal of Invertebrate Pathology 134:23-26.
Cho et al. (2017). The Plant Journal. 90:708-719.
Choi et al. (2012). J Insect Physiol. 58:1159-1165.
Choi et al. (2019). J Economic Entomol. 112:434-439.
Christiaens and Smagghe (2014). Current Opinion in Insect Science 6:15-21.
Christiaens et al. (2016). Scientific Reports 6:38836 doi: 10.1038/srep38836.
Christiaens et al. (2018). Front. Physiol. 9:316.doi: 10.3389/fphys.2018.00316.
Christiaens et al. (2020). Frontiers in Plant Sci. 11:451. doi: 10.3389/fpls.2020.00451.
Christou and Klee, (2004) Handbook of Plant Biotechnology, John Wiley and Sons.
Chukwurah et al. (2019). Plant Biotechnology Reports 13:591-601.
Chung et al. (2006). BMC Genomics 7:120.
Chung et al. (2021). Current Opinion Plant Biol. 60:102029.
Ciofini et al. (2022). Plants 2022, 11, 1856.
Cisneros and Carbonell (2020). Plants 9, 669; doi: 10.3390/plants9060669.
Cisneros et al. (2022). Plant J. 110:1166-1181.
Clough and Bent (1998). Plant J. 16:735-743.
Cockram et al. (2007). J Exp Bot. 58:1231-44.
Coleman et al. (2015). Journal of Experimental Botany 66:541-548.
Cooper et al. (2018). Pest Manag Sci 75:18-28.
Cooper et al. (2020). Insects 2020, 11, 652; doi: 10.3390/insects11100652.
Corrado and Karali (2009). Biotechnol. Adv. 27:733-743.
Coy et al. (2012). J. Appl. Entomol. 136:741-748.
Cruz et al. (2018). Insects 2018, 9, 96; doi: 10.3390/insects9030096.
Cui et al. (2022). Cell Reports 41, 111527.
Cuperus et al. (2010) Nature Structural and Molecular Biology, 17:997-U111.
Dabrowska-Bronk et al. (2015). Mol Plant Pathol 16:334-348.
Dalakouras et al. (2018). Front. Plant Sci. 9:1253. doi: 10.3389/fpls.2018.01253.
Dalakouras et al. (2020). Plant Physiology 182:38-50.
Dalzell et al. (2011). PLoS Negl Trop Dis 5 (6): e1176. doi: 10.1371/journal.pntd.0001176.
Danchin et al. (2013). PLoS Pathog 9 (10): e1003745. doi: 10.1371/journal.ppat. 1003745.
Dang et al. (2022). J. Agric. Food Chem. 70:373-380.
Darlington et al. (2022). Insects 2022, 13, 57.
Davies et al. (1996). J Biol Chem 271:30677-30684.
Degnan et al. (2022). Mol Plant Pathol. 24:191-207.
Deng et al. (2022). International Journal of Molecular Sciences, 23 (2): 696.
De Souza Junior et al. (2013). PLoS ONE 8 (12): e85364. doi: 10.1371/journal.pone.0085364.
Dhandapani et al. (2019). Scientific Rep 9:8775 doi.org/10.1038/s41598-019-45019-z.
Dhandapani et al. (2020). Journal of Pest Science 93:823-832.
Dhandapani et al. (2022). J Agric Food Chem. 70:6634-6643.
Dhatwalia et al. (2021). Pest Manag Sci 78:803-811.
Diamos and Mason (2019). Front. Plant Sci. 9:1974. doi: 10.3389/fpls.2018.01974.
Dias et al. (2019). Front. Physiol. 10:794. doi: 10.3389/fphys.2019.00794.
Diaz-Pendon et al. (2007). Plant Cell 19:2053-2063.
Ding et al. (2021). Int. J. Mol. Sci. 2021, 22, 10057.
Dinh et al. (2014). Phytopathology 104:1098-1106.
Di Prisco et al., (2016). Proc. Nat. Acad. Sci. USA 113:3203-3208.
Dong et al. (2016). Scientific Reports 6:35750 doi: 10.1038/srep35750.
Dong et al. (2023). Insect Mol Biol. 32:1-10.
Dou et al. (2020). Plant Biotechnology Journal 18:11-13.
Drinnenberg et al. (2009) Science 326:544-550.
Du et al. (2012). J Insect Physiol. 58:1413-1423.
Du et al. (2017). Insect Biochem Mol Biol. 87:154-164.
Dubey et al. (2017). Pesticide Biochemistry and Physiology 142:53-58.
Du Plessis et al. (2020). Insects 11, 228. doi: 10.3390/insects11040228.
Dumas et al. (2015). Genetica 143:305-316.
Dunwell (2000). J Exp Botany 51Spec No: 487-496.
Dutta et al. (2015). Front. Microbiol.6:260.doi: 10.3389/fmicb.2015.00260.
Duyvesteijn et al. (2005). Molecular Microbiology 57:1051-1063.
Ebhardt et al. (2005). Proc. Nat. Acad. Sci. USA 102:13398-13403.
Eldred et al. (2014). J Muscle Res Cell Motil 35:211-223.
Ellango et al. (2018). Florida Entomologist-Vol 101, No. 1.
Ellerstrom et al. (1996). Plant Mol. Biol. 32:1019-1027.
Elling et al. (2013). Phytopathology 103:1092-1102.
Erdmann and Picard (2020). PLOS Genetics, 16 (10): e1009034.
Estep et al. (2016). Journal of Insect Physiology 90:17-26.
Fairbairn et al. (2007). Planta 226:1525-1533.
Fan et al. (2016). Proc. Nat. Acad. Sci. USA 113:15144-15149.
Fang et al. (2016). Frontiers Microbiol. 7:1329. doi: 10.3389/fmicb.2016.01329.
Fehr (1987). In: Breeding Methods for Cultivar Development, Wilcox J. ed. American Society of Agronomy, Madison Wis.
Fernandes et al. (2016). Genetics and Molecular Research 15 (2): gmr. 15027941.
Fernandez et al. (2021). Plant Biotechnol J. doi.org/10.1111/pbi.13685.
Finetti et al. (2023). Pesticide Biochemistry and Physiology 189 (2023) 105295
Firmino et al. (2013). PLoS ONE 8 (12): e85079. doi: 10.1371/journal.pone.0085079.
Firmino et al. (2020). Front. Physiol. 11:591569. doi: 10.3389/fphys.2020.591569.
Fisher et al. (2012). Nature 484:186-194.
Fishilevich et al. (2016). Pest Manag Sci 72:1652-1663.
Fishilevich et al. (2019). Insect Biochem Mol Biol 104:20-29.
Flynt (2020). Pest Manag Sci 77:2179-2187.
Fu et al. (2016a). Pest Manag Sci 72:1231-1241.
Fu et al. (2015). Scientific Reports 5:18124| DOI: 10.1038/srep18124.
Fu et al. (2016b). Gene 581:170-177.
Fu et al. (2019). Pest Manag Sci 76:1761-1771.
Fu et al. (2020). Plant Cell Environ. 2022:1-12.
Fujimoto and Iwakawa (2023). J Biochem 174:491-499.
Furlong et al. (2013). Annu. Rev. Entomol. 58:517-41.
Fusaro et al. (2006). EMBO Reports, 7:1168-1175.
Gaffar and Koch (2019). Viruses 2019, 11, 673; doi: 10.3390/v11070673.
Gaines et al. (2010). Proc. Nat. Acad. Sci. USA 107:1029-1034.
Galdeano et al. (2017). PLoS ONE 12 (3): e0171847. doi: 10.1371/journal.pone.0171847 Gan and Amasino (1995). Science 270:1986-1988.
Gao et al. (2020). J. Agric. Food Chem. 70:12408-12417.
Garbian et al. (2012). PLoS Pathog 8 (12): e1003035. doi: 10.1371/journal.ppat.1003035.
Garcia et al. (2017). PLoS ONE 12 (12): e0189600.
Gebremichael et al. (2021). Plants 2021, 10, 650.
Ghag et al. (2014). Plant Biotechnology Journal 12:541-553.
Ghag (2017). Physiological and Molecular Plant Pathology 100:242-254.
Ghodke et al. (2019). Scientific Reports 9:11898 doi.org/10.1038/s41598-019-47357-4.
Ghosh et al. (2018). J. Vis. Exp. (135), e57390, doi: 10.3791/57390 (2018).
Ghosh et al. (2018). J. Vis. Exp. 2018, 135, e57390.
Gill et al. (2018). The Plant Journal 93:894-904.
Gillet et al. (2017). Front. Physiol. 8:256. doi: 10.3389/fphys.2017.00256.
Gleave (1992). Plant Mol Biol 20:1203-1207.
Goodfellow et al. (2019). Plants 2019, 8, 572; doi: 10.3390/plants8120572.
Gong et al. (2011). Pest Manag Sci 67:514-520.
Gong et al. (2014). PLoS ONE 9 (8): e102823. doi: 10.1371/journal.pone.0102823.
Gong and Zhang (2021). Developmental and Comparative Immunology 115:103907.
Gong et al. (2021). Pest Manag Sci 78:1128-1137.
Goto et al. (2007). Plant Cell Physiol. 48:1050-1060.
Gouin et al. (2017). Sci Rep 7:11816.
Govindarajulu et al. (2015). Plant Biotechnology Journal 13:875-883.
Greene et al. (1976). Journal of Economic Entomology, 69:487-488.
Griebler et al. (2008). Journal of Insect Physiology 54:997-1007.
Grover et al. (2019). Journal of Asia-Pacific Entomology 22:56-62.
Gu et al. (2019). Pesticide Biochemistry and Physiology 153:36-46.
Guan et al. (2018). J. Biol. Chem. 293:6011-6021.
Guan et al. (2019). Front. Physiol. 10:1368. doi: 10.3389/fphys.2019.01368.
Guo et al. (2014). PLoS ONE 9 (5): e97410. doi: 10.1371/journal.pone.0097410.
Guo et al. (2015a). Pesticide Biochemistry and Physiology 123:64-73.
Guo et al. (2015b). Insect Biochem Mol Biol 63:23-33.
Guo et al. (2015c). Scientific Reports 5:13728 doi: 10.1038/srep13728.
Guo et al. (2016). Curr. Genom. 17:476-489.
Guo et al. (2018). J. Agric. Food Chem. 66:11990-11999.
Guo et al. (2021). Insect Sci. doi: 10.1111/1744-7917.12899.
Guo et al. (2023). Pesticide Biochem Physiol 193:105428.
Guo et al. (2023b). International Journal of Biological Macromolecules 230 (2023) 123123.
Guo and Ding (2002). EMBO J. 21:398-407.
Gupta et al. (1988). Plant Mol. Biol. 10:215-224.
Gurusamy et al. (2020a). Arch. Insect Biochem. Physiol. 2020; 104:e21677.
Gurusamy et al. (2020b). Arch. Insect Biochem. Physiol. 2020; 104:e21678.
Hafeez et al. (2022). Front. Physiol. 13:884447. doi: 10.3389/fphys.2022.884447.
Hafeez et al. (2023). Front. Plant Sci. 13:1079442. doi: 10.3389/fpls.2022.1079442.
Hajeri et al. (2014). J Biotechnol 176:42-49.
Han et al. (2017). Int J Mol Sci. 18:1874.
Harris and Stone (2009). Pp 655-662 in In Chemistry, Biochemistry, and Biology of 1-3 Beta Glucans and Related Polysaccharides Ed. Stone and Clarke.
He et al. (2018). Pest Manag Sci 75:1370-1382.
He et al. (2019). Front. Plant Sci. 10:1362. doi: 10.3389/fpls.2019.01362.
He et al. (2020a). Journal of Experimental Botany 71:2670-2677.
He et al. (2020b). Insects 2020, 11, 449; doi: 10.3390/insects11070449.
He et al. (2022). Pesticide Biochemistry and Physiology 184: e105121.
Head et al. (2017). Pest Manag Sci 73:1883-1899.
Helliwell and Waterhouse (2005). Methods in Enzymology 392:24-35.
Henderson et al. (2006). Nature Genetics 38:721-725.
Herranz et al. (2004). Gene Expression Patterns 4:183-190.
Hershey and Stoner, (1991) Plant Molecular Biol. 17:679-690.
Hewezi and Baum (2015). Advances in Botanical Research 73:221-239.
Himber et al. (2003). EMBO J. 22:4523-4533.
Hinchee et al. (1988). Biotechnology 6:915-922.
Hofle et al. (2020). RNA Biology 17:463-473.
Hollowell et al. (2023). Agr Forest Entomol. 25:272-284.
Holtof et al. (2019). Cell Tissue Res. 2019:1-18. doi.org/10.1007/s00441-019-03031-9.
Hoppenau et al. (2014). Environmental and Experimental Botany 108:14-22.
Horn et al. (2022). Advanced Genetics 2022, 3, 2100064.
Horvath et al. (2000). Proc. Natl. Acad. Sci. U.S.A. 97:1914-1919.
Hough et al. (2022). Front. Bioeng. Biotechnol. 10:980592.
Hsieh et al. (2009). Plant Physiology, 151:2120-2132.
Hu et al. (2003). Molec Endocrinol 17:716-731.
Hu et al. (2014). PLoS ONE 9 (6): e100046. doi: 10.1371/journal.pone.0100046.
Hu et al. (2015). Frontiers in Chemistry Vol 3 Article 1. doi:
10.3389/fchem.2015.00001.
Hu et al. (2016). Scientific Reports 6:30542 doi: 10.1038/srep30542.
Hu et al. (2019). PLoS ONE 14 (1): e0210491.
Hu et al. (2020). Scientific Reports (2020) 10:11139.
Hu et al. (2020b). Molecular Plant Pathology. 2020; 21:794-807.
Huang et al. (2006). Proc Natl Acad Sci USA 103:14302-14306.
Huang et al. (2014). Eur J Plant Pathol 138:181-193.
Huang et al. (2015). Insect Science (2019) 26, 68-75, doi 10.1111/1744-7917.12513.
Hunter et al. (2010). PLoS Pathog 6 (12): e1001160. doi: 10.1371/journal.ppat.1001160.
Hussain et al. (2019). Transgenic Res 28:151-164.
Huvenne and Smagghe (2010). J Insect Physiol. 56:227-235.
Ibrahim et al. (2011). Experimental Parasitology 127:90-99.
Ibrahim et al. (2019). Plant Physiol. Biochem. 141:332-342.
Iqbal et al. (2020). Front. Plant Sci. 11:328.doi: 10.3389/fpls.2020.00328.
Israni and Rajam (2017). Insect Mol Biol. 26:164-180.
Itsathitphaisarn et al., (2017). Journal of Invertebrate Pathology 147:76-85.
Ivashuta et al. (2015). RNA 21:840-850.
Jahan et al. (2015). Journal of Experimental Botany 66:2785-2794.
Jain et al. (2020). Pest Manag Sci 77:2188-2196.
Jaiwal et al. (2020). J Biosci 45:109.
Jayachandran et al. (2013). Insect Biochem. Mol. Biol. 43:398-406.
Jayaram et al. (2008). PLoS ONE 3: e1964. doi: 10.1371/journal.pone.0001964.
Jeddeloh et al. (1999). Nat. Genet. 22:94-97.
Jefferson et al. (1987). EMBO J 6:3901-3907.
Jekayinoluwa et al. (2021). Plants 10, 613.doi.org/10.3390/plants10040613.
Jepson et al. (1994). Plant Mol. Biol. 26:1855-1866.
Jiang et al. (2022). Int. J. Mol. Sci. 2022, 23, 444.
Jiao et al. (2017). Environmental Microbiology 19:4177-4189.
Jin et al. (2015). Plant Biotechnology Journal 13:435-446.
Joga et al. (2016). Frontiers in Physiology 7:553. doi: 10.3389/fphys.2016.00553.
Johnson (1987). J. Trop. Insect Sci. 8:543-549.
Johnson et al. (2018). Mycotoxin Res 34:29-37.
Jones et al. (2013). Mol. Plant Pathol. 14:946-961.
Jones and Fosu-Nyarko (2014). Ann Appl Biol 164:163-181.
Jonjaroen et al. (2024). Rev Aquac. 16:1647-1673.
Joseph et al. (2012). Molecular & Biochemical Parasitology 186:51-59.
Joshi et al. (2019). Scientific Reports 9:17404 doi.org/10.1038/s41598-019-53485-8.
Joshi et al. (2020). Plant Science 298 (2020) 110592.
Jung and Muller (2009). Trends in Plant Science 14:563-573.
Kalyandurg et al. (2021). Phytopathology 111:2168-2175.
Kaplanoglu et al. (2022). Int. J. Mol. Sci. 23, 3918. doi.org/10.3390/ijms23073918.
Kaur et al. (2021). Physiol Mol Biol Plants 27:2357-2377.
Kay et al. (1987) Science 236:1299-1302.
Kebede and Fite (2022). Front. Mol. Biosci. 9:944774. doi: 10.3389/fmolb.2022.944774.
Khajuria et al. (2015). Insect Biochemistry and Molecular Biology 63 (2015) 54e62.
Khalil et al. (2023). Eur. J. Entomol. 120:15-25.
Khalil et al. (2023b). Acta Tropica 243 (2023) 106938.
Khila and Grbic (2007). Dev Genes Evol 217:241-251.
Khan (2017). Sci Rep 7, 40025
Killiny et al. (2014). PLoS ONE 9 (10): e110536. doi: 10.1371/journal.pone.0110536.
Kim et al. (2019) J. Gen. Plant Path. https://doi.org/10.1007/s10327-019-00865-7
Kim et al. (2023). J. Agric. Food Chem. 71:19302-19311.
Kishore and Somerville (1993). Curr Opin Biotechnol. 4:152-158.
Klink et al. (2009). Planta (2009) 230:53-71.
Knorr et al. (2018). Scientific Reports 8:2061 doi: 10.1038/s41598-018-20416-y.
Koch et al. (2013). Proc Natl Acad Sci USA. 110:19324-19329.
Koch et al. (2016). PLoS Pathog 12 (10): e1005901. doi: 10.1371/journal.ppat. 1005901.
Koch et al. (2019). Molecular Plant Pathology 20:1636-1644.
Koelle et al. (1992). Proc Natl Acad Sci USA 89:6167-6171.
Kohli et al. (2018). Parasitology 145:1896-1906.
Kola et al. (2015). Science China 62:1703-1706.
Kolgy et al. (2021). ACS Appl Bio Mater. 4:5145-5157.
Kolliopoulou and Swevers (2014). Current Opinion in Insect Science 6:28-34.
Kondylis and Rabouille (2009). FEBS Lett 583:3827-3838.
Kong et al. (2014). Insect Molecular Biology 23:632-643.
Kotwica-Rolinska et al. (2013). Insect Biochemistry and Molecular Biology 43.522e532.
Koulagi et al. (2020). Plant Cell, Tissue and Organ Culture 143:345-361.
Koziel et al. (1996). Plant Mol. Biol. 32:393-405.
Kozomara et al. (2019). Nucleic Acids Res. 47: D155-D162.
Kumar et al. (2009). J Insect Physiol 55:273-278.
Kumar et al. (2016). International Journal of Biological Macromolecules 86:89-95.
Kumar et al. (2017). J Gen Plant Pathol 83:91-97.
Kumar et al. (2022). Planta (2022) 256:74-doi.org/10.1007/s00425-022-03977-1.
Kunte et al. (2019). Pest Manag. Sci. 76:26-41.
Kunte et al. (2020). Pest Manag Sci 76:26-41.
Kwon et al. (2013). Pestic Biochem Physiol 105:69-75.
Kwon et al. (2016). Pesticide Biochemistry and Physiology 130:1-7.
Kyndt et al. (2013). Nematology 15:519-528.
Kyre et al. (2019). Scientific Reports 9:5640 doi.org/10.1038/s41598-019-42072-6.
Lacroix et al. (2008). Proc. Natl. Acad. Sci. U.S.A. 105:15429-15434.
Lan et al. (2018). Arch. Insect Biochem. Physiol. 97: e21438.
Lau et al. (2001) Science 294:858-862.
Lazarides et al. (1997) CSIRO Handbook of Australian Weeds, CSIRO Publishing.
Lee et al. (2006). Biochimica et Biophysica Acta 1760:125-133.
Leigh et al. (2015). Aquaculture 437:360-365
Leonard et al. (2020). Science 367:573-576.
Li and Bender (2000). Development 127:2897-2905.
Li et al. (1996). FEBS Lett. 379:117-121.
Li et al. (2010). Planta 232:775-785.
Li et al. (2011). PLoS ONE 6 (3): e17788. doi: 10.1371/journal.pone.0017788.
Li et al. (2013). PLoS ONE 8 (6): e65931. doi: 10.1371/journal.pone.0065931.
Li et al. (2015). PLoS ONE 10 (6): e0129351. doi: 10.1371/journal.pone.0129351.
Li et al. (2015b). Int. J. Biol. Sci. 11:1073-1087.
Li et al. (2015c). J. Appl. Entomol. 139:432-445.
Li et al. (2017). Plant J. 90:654-670.
Li et al. (2017b). Scientific Reports 7:13126| DOI: 10.1038/s41598-017-13024-9.
Li et al. (2018). Insect Science 25:45-56.
Li et al. (2022). Front. Bioeng. Biotechnol. 10:963026. doi: 10.3389/fbioe.2022.963026
Lilley et al. (2012). Parasitology 139:630-640.
Lim et al. (2016). J. Insect Physiol. 85:86-93.
Lim et al. (2020). Plant Biotechnology Reports 14:643-662.
Lisei-de-Sa et al. (2021). Planta (2021) 254:121-doi.org/10.1007/s00425-021-03776-O.
Liu et al. (2014). Insect Biochemistry and Molecular Biology 55:51-60.
Liu et al. (2015). Int J Biol Sci. 11:67-74.
Liu et al. (2015b). PLoS ONE doi: 10.1371/journal.pone.0128892.
Liu et al. (2020). Plant Cell, 32, 3059-3080.
Liu et al. (2020b). Science of the Total Environment 706 (2020) 135872.
Liu et al. (2021). Pest Manag Sci 78:3815-3822.
Liu et al. (2022). Int. J. Mol. Sci. 2022, 23, 8239. doi.org/10.3390/ijms23158239.
Lopez et al. (2019). Scientific Reports 9:4038 doi.org/10.1038/s41598-019-39666-5.
Lopez-Gomollon and Baulcombe (2022). Nature Reviews Molec Cell Biol 23:645-662.
Lopez-Marquez et al. (2021). Journal of Experimental Botany, 72:7316-7334.
Lourenco-Tessutti et al. (2015). Phytopathology 105:628-637.
Lu et al. (2009). FEBS Journal 276:3110-3123.
Lu et al. (2015). J. Econ. Entomol. 108:1344-1353.
Lu et al. (2015b). Gene 570:264-271.
Lu et al. (2020a). Pestic. Biochem. Physiol. 165:104555.doi: 10.1016/j.pestbp.2020.104555.
Lu et al. (2020b). Insects 11:34. doi: 10.3390/insects11010034.
Lu et al. (2021a). Insect Sci. 28:509-520.
Lu et al. (2021b). J. Pest. Sci. 94:1473-1486.
Lucena-Leandro et al. (2022). Int. J. Mol. Sci. 23:15836. doi.org/10.3390/ijms232415836.
Luo et al. (2012). Plant Molecular Biology, 80:117-129.
Luo et al. (2019). Fungal Genetics and Biology 126:25-36.
Lyu et al. (2023). Pest Manag Sci 79:1518-1527.
Ma et al. (2020). Journal of Integrative Agriculture 19:428-437.
Ma et al. (2023). Journal of Pest Science 96:181-193.
Macedo et al. (2017). Biotechnology Research and Innovation (2017) 1, 72-86.
Machado and Andrew (2000. J Cell Biol 151:639-651.
Mahfouz et al. (2006). Plant Cell 18:477-490.
Mahto et al. (2020). Plant Molecular Biology 104:381-395.
Maksimov et al. (2022). Applied Biochem Microbiol. Vol. 58, Suppl. 1, pp. S16-S31.
Maktura et al. (2021). J Appl Entomol. 145:740-756.
Mamta et al. (2016). Plant Mol Biol 90:281-292.
Manavella et al. (2012 Proc. Natl. Acad. Sci. USA, 109:2461-2466.
Mani et al. (2020). Int. J. Mol. Sci. 2020, 21, 6626; doi: 10.3390/ijms21186626.
Mann et al. (2023). Int. J. Mol. Sci. 2023, 24, 12391.
Mao et al. (2007). Nature Biotech 25:1307-1313.
Mao et al. (2011). Transgenic Res. 20:665-673.
Mao and Zeng (2014). Transgenic Res. 23:145-152.
Mao et al. (2015). Pesticide Biochemistry and Physiology 118:71-76.
Martinez et al. (2021). Pesticide Biochemistry and Physiology 175 (2021) 104853.
Matsuoka et al. (1994). Plant J. 6:311-319.
McCahill et al. (2002). Molecular Pharmacology 62:1261-1273.
Mccullough and Schuler (1997). Nucl Acids Res. 25:1071-1077.
McGraw et al. (2022). ACS Omega 2022, 7, 10933-10943.
McGruddy et al. (2024). Pest Manag Sci 2024; 80:4770-4778.
McHale et al. (2013). Plant Journal, 76:519-529.
Mclaughlin et al. (2023). Plants 2023, 12, 3822.
McLoughlin et al. (2018). Can. J. Plant Pathol. 40:325-335.
McRae et al. (2023). Mol Plant Pathol. 24:1168-1183.
Mehlhorn et al. (2021). Frontiers in Zoology 18:60 doi.org/10.1186/s12983-021-00444-7.
Mehlhorn et al. (2021b). Pesticide Biochem Physiol 176:104870.
Meier et al. (1997). FEBS Lett. 415:91-95.
Melamed-Bessudo et al. (2012) Proc. Natl. Acad. Sci. USA 109 (16): e981-988.
Meng and Lemaux (2003). Plant Molecular Biology Reporter 21:65-71.
Meng et al. (2020). Insect Science 27:113-121.
Merzendorfer et al. (2000). Biochim. Biophys. Acta 25:369-379.
Miyata et al. (2014). PLoS ONE 9 (7): e101661. doi: 10.1371/journal.pone.0101661.
Mlotshwa et al. (2008). Plos One, 3 (3): e1755.
Molnar et al. (2010). Science 328:872-875.
Mondal et al. (2021). BMC Genomics (2021) 22:42 doi.org/10.1186/s12864-020-07322-2.
Montavon et al. (2018). The Plant Journal 95:204-218.
Mulot et al. (2018). Front. Microbiol. 9:457.doi: 10.3389/fmich.2018.00457.
Moreira-Pinto et al. (2021). Pest Manag Sci 77:4054-4063.
Murtaza et al. (2022). Molecular Biotechnology 64:1152-1163.
Mutti et al. (2006). J Insect Sci 6:38.
Mutti et al. (2008). Proc. Natl. Acad. Sci. USA 105:9965-9969.
Nagano et al. (2014). Nucleic Acids Research 42:1845-1856.
Nguyen et al. (2018). Reviews in Aquaculture 10:573-584.
Nguyen et al. (2022). Nematology 24:571-588.
Nien et al. (2024). Plant Cell Physiol. 00 (00): 1-10 (2024) doi.org/10.1093/pcp/pcae012.
Nitnavare et al. (2021). Frontiers in Plant Sci. 12:673576. doi: 10.3389/fpls.2021.673576.
Niu et al. (2012). Eur J Plant Pathol 134:131-144
Niu et al. (2016). Sci Rep 6:19443.
Niu et al. (2017). Scientific Reports 7:12591 doi: 10.1038/s41598-017-12638-3.
Niu et al. (2018). Pest Manag Sci 74:2680-2687.
Niu et al. (2019). Pest Manag Sci 75:2873-2881.
Niu et al. (2021). Current Opinion in Biotechnology 70:204-212.
Nowara et al. (2010). The Plant Cell 22:3130-3141.
Oakeshott et al. (2005). Pp 309-381 in: Gilbert et al (Eds.), Comprehensive Molecular
Insect Science, vol. 5. Elsevier Ltd. Oxford, UK.
Olive et al. (1989) Plant Mol Biol 12:525-538.
Ongvarrasopone et al. (2008). Mar Biotechnol 10:374-381.
Ongvarrasopone et al. (2010). Antiviral Research 88:116-118.
Ongvarrasopone et al. (2011). Arch Virol 156:1117-1123.
Ouyang et al. (2023). PLoS Pathog 19 (6): e1011463.
Overton et al. (2021). Crop Protection 145 (2021) 105641.
Padidam (2003). Transgenic Res. 12:101-109.
Padilla-Roji et al. (2023). Int. J. Mol. Sci. 2023, 24, 9082.
Palli et al. (1992). Developmental Biol 150:306-315.
Palli (2023). Front. Insect Sci. 3:1209478. doi: 10.3389/finsc.2023.1209478.
Palli et al. (2014). Current Opinion in Insect Science 6:1-8.
Pampolini and Rieske (2023). Forests 2023, 14, 1853. doi.org/10.3390/f14091853.
Panwar et al. (2013a). The Plant Journal 73:521-532.
Panwar et al. (2013b). Plant Mol Biol 81:595-608.
Panwar et al. (2018). Plant Biotechnology Journal 16:1013-1023.
Papolu et al. (2013). PLoS ONE 8 (11): e80603. doi: 10.1371/journal.pone.0080603.
Parent et al. (2015). Plant Journal, 81:223-232.
Park and Vatanparast (2022). Arch Insect Biochem Physiol. 2022; 110: e21897.
Parsons et al. (2018). Biomacromolecules 19:1111-1117.
Parvy et al. (2014). Development 141:3955-3965.
Patel et al. (2010). Journal of Experimental Botany 61:235-248.
Patel et al. (2021). Plant Physiology, 185:1764-1782.
Peccoud et al. (2011) PLoS ONE 6: e16765.
Peng et al. (2020a). Pesticide Biochem Physiol 162:86-95.
Peng et al. (2020b). Insect Biochem Mol Biol 125:103440.
Pepperkok et al. (1993). Cell 74:71-82.
Perez et al. (2020). Plant Pathology 70:417-425.
Perrin et al. (2000). Mol Breed 6:345-352.
Petek et al. (2020). Front. Plant Sci. 11:1250. doi: 10.3389/fpls.2020.01250.
Pinheiro et al. (2018). PLoS ONE 13 (8): e0201849.
Pitino et al. (2011). PLoS ONE 6, e25709.
Pitino and Hogenhout (2012). Molecular Plant-Microbe Interactions 26:130-139.
Potenza et al. (2004). In Vitro Cell Dev. Biol. Plant 40:1-22.
Powell et al. (1996). Vaccines 183, Abstract.
Powell et al. (2016). Pest Manag Sci 73:53-63.
Preiss et al. (1987). In: Tailoring Genes for Crop Improvement (Bruening et al. eds.), Plenum Press, S. 133-152.
Qi et al. (2015). Bulletin Entomol Res 105:555-565.
Qi et al. (2018). Plant Biotechnology Journal 16:797-807.
Qiao et al. (2014). Developmental Compar Immunol 44:94-99.
Qiao et al. (2021). Plant Biotech J. (2021), pp. 1-13, doi: 10.1111/pbi.13589.
Qiao et al. (2023). New Phytologist 240:68-79.
Qiao et al. (2023b). Pesticide Biochemistry and Physiology 194 (2023) 105481.
Qin et al. (2017). Plant Physiology, 174:1067-1081.
Qin et al. (2022). Insects 2022, 13, 755. doi.org/10.3390/insects13080755.
Rafaeli and Jurenka (2003). In: Blomquist, G. J. Vogt, R. (Eds.), Pheromone Biochemistry and Molecular Biology. Academic Press, New York, pp. 107-136.
Rajagopal et al. (2002). J. Biol. Chem. 277:46849-46851.
Ramkumar et al. (2021). Front. Physiol. 12:742871. doi: 10.3389/fphys.2021.742871.
Ramos-Carreno et al. (2021). Beilstein J. Org. Chem. 17, 1360-1373.
Rana et al. (2020). Front. Plant Sci. 11:427.doi: 10.3389/fpls.2020.00427.
Rangasamy and Siegfried (2011). Pest Manag Sci 68:587-591.
Raruang et al. (2023). Front. Plant Sci. 14:1150086. doi: 10.3389/fpls.2023.1150086.
Ray et al. (2022). Front. Fungal Bio. 3:977502. doi: 10.3389/ffunb.2022.977502.
Regmi et al. (2023). BMC Genomics (2023) 24:582 doi.org/10.1186/s12864-023-09686-7.
Reis et al. (2022). Pesticide Biochemistry and Physiology 186:105166.
Ren et al. (2002). Arch. Insect Biochem. Physiol. 51:103-110.
Ribeiro et al. (2022). Int. J. Mol. Sci. 2022, 23, 13713.
Ricci et al. (2021). J Gen Plant Pathology doi.org/10.1007/s10327-019-00865-7.
Robinson et al. (2014). J. Plant Biochem. Biotechnol. 23:231-237.
Riddiford et al. (2000). Vitamins and Hormones, 2000, 60:1-73.
Roderick et al. (2018). Plant Biotechnology Journal 16:520-529.
Rodrigues et al. (2017a). Scientific Reports 7:8913 doi: 10.1038/s41598-017-08813-1.
Rodrigues et al. (2017b). Scientific Reports 7:7379 doi: 10.1038/s41598-017-07605-x.
Rodrigues et al. (2018). Scientific Reports 8:5020 doi: 10.1038/s41598-018-23216-6.
Rodrigues et al. (2021). Front. Plant Sci. 12:728652. doi: 10.3389/fpls.2021.728652.
Rodriguez-Cabrera et al. (2010). Environmental Microbiology 12:2894-2903.
Rodriguez-de la Noval et al. (2019). Scientific Reports 9:2630 doi.org/10.1038/s41598-019-38734-0.
Rosa et al. (2018). Annu. Rev. Phytopathol. 2018. 56:581-610.
Ross et al. (2024). Biotechnol Bioeng. 121:3582-3599.
Rosso et al. (2005). MPMI 18:615-620.
Ruiz-Jimenez et al. (2021). J. Fungi 2021, 7, 735. doi.org/10.3390/jof7090735.
Rutter and Innes (2018). Current Opinion in Plant Biology 44:16-22.
Saberi et al. (2024). Insects 2024, 15, 58. doi.org/10.3390/insects15010058.
Saini et al. (2018). PLos One 13 (3): e0194150.
Saito et al. (2022). Journal of General Plant Pathology 88:203-206.
Sakurai et al. (2021). Proc. Natl. Acad. Sci. USA, 118 (31) e2102889118.
Salvador et al. (2021). Neotrop Entomol. 50:121-128.
Sanan-Mishra et al. (2021). Frontiers in Plant Science, 12:610283.
San Miguel and Scott (2015). Pest Manag Sci 72:801-809.
Sarathi et al. (2008). Mar Biotechnol 10:242-249.
Sarkar and Kalia (2023). Biologia 78:429-442.
Schaffner (1980). Proc. Natl. Acad. Sci. U.S.A. 77:2163-2167.
Schoville et al. (2018). Scientific Reports (2018) 8:1931 doi: 10.1038/s41598-018-20154-1.
Scott et al. (2013). Journal of Insect Physiology 59:1212-1221.
Secic and Kogel (2021). Current Opinion in Biotechnology 70:136-142.
Seddas et al. (2004). Virology 325:399-412.
Shabab et al. (2014). FEBS Journal 281:2769-2783.
Shahid et al. (2018). Nature, 553:82-85.
Shakesby et al. (2009). Insect Biochemistry and Molecular Biology 39:1-10.
Shen et al. (2015) Frontiers in Plant Science 6:281.
Shi et al. (2018). Front. Physiol. 9:575.doi: 10.3389/fphys.2018.00575.
Shi et al. (2022). Journal of Insect Science 22:1-13.
Shiina et al. (1997). Plant Physiol. 115:477-483.
Shivakumara et al. (2017). Front. Plant Sci. 8:473.doi: 10.3389/fpls.2017.00473.
Shpak et al. (2017). RNA Biology 14:1766-1775.
Shukla et al. (2016). RNA Biology 13:656-669.
Shure et al. (1983). Cell 35:225-233.
Sindhu et al. (2009). Journal of Experimental Botany 60:315-324.
Silver et al. (2021). Pest Manag Sci 77:2645-2658.
Singewar and Fladung (2023). Functional & Integrative Genomics 2023, 23:185.
Singh et al. (2013). Journal of Insect Science: Vol. 13 Article 69.
Singh et al. (2020). Plant Molecular Biology Reporter 38:419-429.
Singh et al. (2023). Scientia Hort. 311:111764.
Slater et al. Plant Biotechnology—The Genetic Manipulation of Plants, Oxford University Press (2003).
Smith et al. (2000). Nature 407:319-320.
Soares et al. (2005). Insect Molecular Biology doi: 10.1111/j.1365-2583.2005.00575.x
Somchai et al. (2016). Aquaculture Reports 3:178-183.
Song and Rossi (2017). Biochemical Journal, 474:1603-1618.
Song and Thomma (2018). Molecular Plant Pathology 19:77-89.
Song et al. (2018). Molecular Plant Pathology 19:2543-2560.
Soreq and Seidman (2001). Nature Review Neurosciences 2:8-17.
Spada et al. (2023). Int. J. Mol. Sci. 2023, 24, 4869.
Spit et al. (2017). Insect Biochemistry and Molecular Biology 81:103-116.
Stalker et al. (1988). Science 242:419-423.
Steeves et al. (2006). Functional Plant Biology 33:991-999.
Stukenbrock and Gurr (2023). Nature 617:31-34.
Su et al. (2017). Genes 2017, 8, 25; doi: 10.3390/genes8010025.
Sui et al. (2009). Insect Mol Biol 18:443-452.
Suksai et al. (2025). Aquaculture 595 (2025) 741570.
Suzuki et al. (2017). PLoS ONE 12: e0180654. doi.org/10.1371/journal.pone.0180654.
Swevers et al. (2013). Insect Molecular Biology 22:668-684.
Talbot et al. (1993). Cell 73:1323-1337.
Tan et al. (2011). Plant Physiol. 156:1577-1588.
Tang et al. (2010). Plant Physiology 153:632-641.
Tang et al. (2010b). Journal of Insect Physiology 56:813-821.
Taochy et al. (2017). Plant Physiology, 175:1424-1437.
Tatineni and Hein (2023). Phytopathology 113:117-141.
Teng et al. (2020). Nature Communications, 11:2912.
Teotia and Tang (2015). Molecular Plant. 8:359-377.
Terenius et al. (2011). J. Insect Physiol. 57:231-245.
Tetorya and Rajam (2021). 3 Biotech (2021) 11:443, doi.org/10.1007/s13205-021-02973-8.
Thakre et al. (2024). Pest Manag Sci 80:2141-2153.
Thakur et al. (2014). PLoS ONE 9 (3): e87235. doi: 10.1371/journal.pone.0087235.
Thatcher et al. (2009). Plant Journal 58:927-939.
Thillet et al. (1988). J. Biol. Chem 263:12500-12508.
Thorat et al. (2024). Plant Cell Reports (2024) 43:3-doi.org/10.1007/s00299-023-03114-6.
Thummel (1995). Cell 83:871-877.
Tian et al. (2009). PLoS One 4: e6225.
Tian et al. (2015). Int J Biol Sci. 11:1296-1305.
Tian et al. (2019). Theoretical and Applied Genetics 132:2651-2662.
Timani et al. (2023). Insects 2023, 14, 418. doi.org/10.3390/insects14050418.
Timmons et al. (2001). Gen 263:103-112.
Tinoco et al. (2010). BMC Biology 2010, 8:27.
Tiwari et al. (2017). Scientific Reports 7:7521; doi: 10.1038/s41598-017-07749-w.
Trewitt et al. (1992). J Mol Evol 34:478-492.
Tzin et al. (2015). Journal of Insect Physiology 79:105-112.
Ufaz et al. (2018). Mol. Syst. Des. Eng., 2018, 3, 38.
Ullah et al. (2020). Insects 11, 22; doi: 10.3390/insects11010022.
Ulmasov et al. (1995). Plant Physiol. 108:919-927.
Ulrich et al. (2015). BMC Genomics (2015) 16:674. doi 10.1186/s12864-015-1880-y.
Upadhyay et al. (2011). PLoS One 11 (5): e0155306. doi: 10.1371/journal.pone.0155306
Urwin et al. (2002). MPMI 15:747-752.
Uyehara and Mckay (2019). Proc Natl Acad Sci USA 116:9893-9902. van den Berg et al. (2008). Biochim Biophys Acta-Gene Reg Mechanisms 1779:668-677.
Vatanparast and Kim (2017). PLoS ONE 12: e0183054.
Vatanparast et al. (2021). Arch Insect Biochem Physiol. 2021:108:e21840.
Velez et al. (2017). Genes 2017, 8, 7; doi: 10.3390/genes8010007.
Velez et al. (2020). Pest Manag Sci 76:1500-1512.
Velez et al. (2023). Pp 1-54 in Advances in Insect Physiol. 65. Ed. Jurat-Fuentes, Academic Press. Doi.org/10.1016/S0065-2806 (23) 00027-9.
Venkatesh et al. (2023). Journal of Integrative Agriculture 22:471-480.
Vetukuri et al. (2021). Crop Breeding and Applied Biotechnology 21 (S): e387921S11, 2021.
Viera et al. (2015). PLoS ONE 10 (12): e0144674. doi: 10.1371/journal.pone.0144674.
Voinnet et al. (2003). Plant J. 33:949-956.
Walawage et al. (2013). BMC Genomics 14:668
Walker et al. (2023). Scientific Reports 13:6477; doi.org/10.1038/s41598-023-33335-4.
Wan et al. (2014). PLoS ONE 9 (1): e86675. doi: 10.1371/journal.pone.0086675.
Wan et al. (2021). Journal of Insect Science, 21:1-12.
Wan et al., (2021b). Journal of Integrative Agriculture 20:646-663.
Wang (1994) Isolation of phloem specific gene promoters for use in genetic engineering of insect resistance in rice. PhD thesis, University of Durham, UK.
Wang et al. (1994) Plant Molecular Biology 24:159-170.
Wang et al. (2008) RNA 14:903-913.
Wang et al. (2010). Insect Mol Biol 19:99-111.
Wang et al. (2012). Eur J Plant Pathol 134:521-532.
Wang et al. (2016). Insect Biochem Mol Biol 77:1-9.
Wang et al. (2016b). Int J Parasitology 46:351-360.
Wang et al. (2016c). Nature Plants 16151 doi: 10.1038/NPLANTS.2016.151.
Wang et al. (2017). Current Opinion in Plant Biology 38:133-141.
Wang et al. (2018). Horticulture Research, 5:62
Wang et al. (2019). Insect Molecular Biology 28:235-245.
Wang et al. (2019b). Scientific Reports 9:1087 doi.org/10.1038/s41598-018-37488-5.
Wang et al. (2020). MPMI 33:1108-1111.
Wang et al. (2022). International Journal of Biological Macromolecules 222:1665-1675.
Wang and Dean (2022). Front. Plant Sci. 13:959641. doi: 10.3389/fpls.2022.959641.
Wang et al. (2023). Phytopath Res 5:2 doi.org/10.1186/s42483-023-00157-1.
Wang et al. (2023b). Phytopath Res. (2023) 5:2, doi.org/10.1186/s42483-023-00157-1.
Wang et al. (2023c). Insect Mol Biol. 32:412-423.
Wang et al. (2024). J. Agric. Food Chem. 72:10936-10943.
Wang and Jin (2017). Trends in Microbiology 25, No 1.
Ward et al. (2013). Weed Technology 27:12-27.
Weber et al. (2011) PloS one. 6 (2), pp. e16765.
Weissbach et al. (1988). In: Methods for Plant Molecular Biology, Academic Press, San Diego, Calif.
Westwood et al. (2018). Weed Sci 66:275-285. doi: 10.1017/wsc.2017.78.
Wieczorek et al. (1989). J. Biol. Chem. 264:11143-11148.
Wieczorek et al. (2003). Journal of Bioenergetics and Biomembranes 35:359-366.
Wieczorek et al. (2009). J Exp Biol 212:1611-1619.
Wijnker et al. (2008). Trends in Plant Science 13:640-646.
Willow and Veromann (2021). Front. Plant Sci. 12:790816. doi: 10.3389/fpls.2021. 790816.
Wood et al. (2009) Plant Biotechnol J. 7:914-924.
Wu et al. (2016). Plant Biotech J. 14:1925-1935.
Wu et al. (2017). PLoS One 12 (12): e0190208.
Wu et al. (2022). Proc Natl Acad Sci USA Vol. 119 No. 15 e2120081119.
Wu et al. (2023). New Phytologist 237:1363-1373.
Wubben et al. (2010). Molecular & Biochemical Parasitology 172:31-40.
Wuriyanghan et al. (2011). PLoS ONE 6 (11): e27736.
Wuriyanghan et al. (2013). PLoS One 6 (11): e27736. doi: 10.1371/journal.pone. 0027736.
Xiao and Lu (2022). International Journal of Biological Macromolecules 219:452-462.
Xie et al. (2005). Proc Natl Acad Sci USA 102:12984-12989.
Xie et al. (2016). Front. Plant Sci. 7 (2016), doi.org/10.3389/fpls.2016.00964.
Xie et al. (2022). Pest Manag Sci 78:4956-4962.
Xiong et al. (2013). Internat J Biol Sci 9:370-381.
Xu et al. (2016). Genes 7, 88; doi: 10.3390/genes7100088.
Xu et al. (2016b). Scientific Reports 6:28976 DOI: 10.1038/srep28976.
Xu et al. (2020). Plant Biotechnology Journal 18:1830-1842.
Xu et al. (2021). Microbiome 9:98. doi.org/10.1186/s40168-021-01066-1.
Xu et al. (2024). Plant Biotechnology Journal 22:262-277.
Xue et al. (2013). Phytopathology 103:175-181.
Yadav et al. (2006). Molecular & Biochemical Parasitology 148:219-222.
Yang et al. (1994) Particle Bombardment Technology for Gene Transfer, Oxford Press, Oxford, England.
Yang et al. (2003). Planta 216:597-603.
Yang et al. (2013). PLoS ONE 8 (7): e69463. doi: 10.1371/journal.pone.0069463.
Yang and Han (2014a). J Integrative Agric. 13:115-123.
Yang and Han (2014b). Austral Entomology 53:83-88.
Yao et al. (2022). Frontiers in Physiology 13:850022doi: 10.3389/fphys.2022.850022.
Ye et al. (2019). Scientific Reports 9:3694| doi.org/10.1038/s41598-019-39837-4.
Yi et al. (2018). BMC Plant Biology. 18:1-13.
Yin et al. (2011). Molecular Plant-Microbe Interactions 24:554-561.
Yin et al. (2015). BMC Genomics (2015) 16:579, DOI 10.1186/s12864-015-1791-y.
Yogindran and Rajam (2016). Insect Biochem Mol Biol 77:21-30.
Yogindran and Rajam (2021). Genomics 113:737-747.
Yoon et al. (2016). Insect Biochemistry and Molecular Biology 78:78-88.
Yoon et al. (2017). Insect Biochemistry and Molecular Biology 90:53-60.
Yoon et al. (2018). Proc Natl Acad Sci USA 115:8334-8339.
Yu et al. (2016). Pest Management Science 72:1090-1098.
Yu et al. (2022). Int. J. Mol. Sci. 2022, 23, 14370.
Yu et al. (2023). ACS Appl. Mater. Interfaces 2023, 15, 48495-48505.
Yue et al. (2016). Plant Biotech J. 14:1747-1755.
Zabala-Pardo et al. (2022). Adv Weed Sci. 2022; 40 (Spec1): e020220096.
Zeng et al. (2021). Insects 12, 935
Zha et al. (2011). PLoS One 6: e20504. doi: 10.1371/journal.pone.0020504.
Zhang and Shi (2002). Cell Death and Differentiation 9:790-800.
Zhang et al. (2006). Genes Dev. 20:3255-3268.
Zhang et al. (2013). Bulletin Entomol Res 103:584-591.
Zhang et al. (2013b). BMC Genomics 14:560.
Zhang et al. (2015a). Science 347:991-994.
Zhang et al. (2015b). Insect Mol Biol 24:155-166.
Zhang et al. (2016). Molecular Plant 9:939-942.
Zhang et al. (2017). Trends Biotech. 35:9. doi.org/10.1016/j.tibtech.2017.04.009 871.
Zhang et al. (2018). Nat Rev Mol Cell Biol. 19:489-506.
Zhang et al. (2019). Insects 10, 415; doi: 10.3390/insects10120415.
Zhang et al. (2020). J Insect Science 20:1-7.
Zhang et al. (2020b). BMC Genomics (2020) 21:155.
Zhang et al. (2022). Nature Communications 13:3926. doi.org/10.1038/s41467-022-31641-5.
Zhang et al. (2022b). Agriculture 12, 2108.
https://doi.org/10.3390/agriculture12122108.
Zhao et al. (2004). Insect Molecular Biology 13:407-412.
Zhao et al. (2013). PLoS ONE 8 (3): e59527. doi: 10.1371/journal.pone.0059527.
Zhao et al. (2016). J Economic Entomol 109:2506-2516.
Zhao et al. (2018). Insect Biochem Mol Biol 92:1e11.
Zhong et al. (2019). Genes 10, 458; doi: 10.3390/genes10060458.
Zhou et al. (2019). Pest Manag Sci 76:712-720.
Zhu and Palli (2020). Ann Rev. Entomol. 65:293-311.
Zhu et al. (2003). Insect Biochem. Mol. Biol. 33:889-899.
Zhu et al. (2011). Pest Manag Sci 67:175-182.
Zhu et al. (2012). PLOS One 7 (6): e38572. doi: 10.1371/journal.pone.0038572.
Zhu et al. (2017). Plant Physiology 175:1853-1863.
Zierold et al. (2005). Molecular Plant Pathology 6:139-151.

1-41. (canceled)

42. A precursor RNA molecule comprising at least one double-stranded RNA region, wherein the double-stranded RNA region comprises:
(i) first a RNA strand of at least 46 contiguous ribonucleotides which comprises a first RNA sequence of at least 44 contiguous ribonucleotides, and
(ii) a second RNA strand of at least 46 contiguous ribonucleotides which comprises a second RNA sequence of at least 44 contiguous ribonucleotides,
wherein the first RNA sequence hybridises to the second RNA sequence by basepairing between the at least 44 contiguous ribonucleotides of the first RNA sequence and the at least 44 contiguous ribonucleotides of the second RNA sequence, forming at least a part within the double-stranded RNA region,
wherein between 10% and 35% of the ribonucleotides of the first RNA sequence and the second RNA sequence, in total, are basepaired in G:U basepairs,
wherein the second RNA sequence is at least 80% identical to a sequence of at least 44 contiguous ribonucleotides which is fully complementary to a first region of a target RNA molecule in an insect cell,
wherein cleavage of the precursor RNA molecule in the insect cell by one or more Dicers produces double-stranded product RNA molecules, each independently consisting of a sense RNA sequence of 21 contiguous ribonucleotides from the first RNA sequence and an antisense RNA sequence of 21 contiguous ribonucleotides from the second RNA sequence,
wherein at least some of the double-stranded RNA molecules have non-overlapping antisense RNA sequences,
wherein ribonucleotides 1 to 19 of the sense RNA sequence basepair with ribonucleotides 1 to 19 of the antisense RNA sequence in each of the double-stranded product RNA molecules,
wherein the 19 basepairs between the sense RNA sequence and the antisense RNA sequence in each of the double-stranded product RNA molecules comprise, independently, 2, 3, 4, 5 or 6 G:U basepairs,
wherein ribonucleotides 20 and 21 of the sense RNA sequence in each of the double-stranded product RNA molecules form a 2-ribonucleotide unpaired 3′ overhang, and wherein ribonucleotides 20 and 21 of the antisense RNA sequence in each of the double-stranded product RNA molecules form a 2-ribonucleotide unpaired 3′ overhang, and
wherein either (iii) at least some of the antisense RNA sequences in the double-stranded product RNA molecules reduce the expression and/or activity of the target RNA molecule in the insect cell, or
(iv) the antisense RNA sequences in the double-stranded product RNA molecules are each, independently, at least 80% identical in sequence to a corresponding region of the complement of the target RNA molecule, or
(v) both (iii) and (iv).

43. A precursor RNA molecule comprising at least one double-stranded RNA region, wherein the double-stranded RNA region comprises:
(i) a first RNA strand of at least 46 contiguous ribonucleotides which comprises a first RNA sequence of at least 44 contiguous ribonucleotides, and
(ii) a second RNA strand of at least 48 contiguous ribonucleotides which comprises a second RNA sequence of at least 46 contiguous ribonucleotides,
wherein the first RNA sequence hybridises to the second RNA sequence by basepairing between at least 42 ribonucleotides of the at least 44 contiguous ribonucleotides of the first RNA sequence and at least 42 ribonucleotides of the at least 46 contiguous ribonucleotides of the second RNA sequence, forming at least a part within the double-stranded RNA region,
wherein 2, 3 or 4 ribonucleotides of the at least 46 contiguous ribonucleotides of the second RNA sequence are non-basepaired and 0, 1 or 2, respectively, ribonucleotides of the at least 44 contiguous ribonucleotides of the first RNA sequence are non-basepaired in the part of the double-stranded RNA region, forming bulges in the part of the double-stranded RNA region,
wherein each of the bulges is immediately flanked by ribonucleotides which are basepaired in the part of the double-stranded RNA region,
wherein the second RNA sequence is at least 80% identical to a sequence of at least 46 contiguous ribonucleotides which is fully complementary to a first region of a target RNA molecule in an insect cell,
wherein cleavage of the precursor RNA molecule in the insect cell by one or more Dicers produces double-stranded product RNA molecules, each independently consisting of a sense RNA sequence of 21 contiguous ribonucleotides from the first RNA sequence and an antisense RNA sequence of 22 contiguous ribonucleotides from the second RNA sequence,
wherein at least some of the double-stranded RNA molecules have non-overlapping antisense RNA sequences,
wherein the antisense RNA sequence of the double-stranded product RNA molecules each comprise at least one of the bulges,
wherein ribonucleotides 20 and 21 of the sense RNA sequence in each of the double-stranded product RNA molecules form a 2-ribonucleotide unpaired 3′ overhang,
wherein ribonucleotides 21 and 22 of the antisense RNA sequence in each of the double-stranded product RNA molecules form a 2-ribonucleotide unpaired 3′ overhang, and
wherein either (iii) at least some of the antisense RNA sequences in the double-stranded product RNA molecules reduce the expression and/or activity of the target RNA molecule in the insect cell, or
(iv) the antisense RNA sequences in the double-stranded product RNA molecules are each, independently, at least 80% identical in sequence to a corresponding region of the complement of the target RNA molecule, or
(v) both (iii) and (iv).

44. A precursor RNA molecule comprising at least one double-stranded RNA region, wherein the double-stranded RNA region comprises:
(a) a RNA first Strand of at least 46 contiguous ribonucleotides which comprises a first RNA sequence of at least 44 contiguous ribonucleotides, and
(b) a second RNA strand of at least 50 contiguous ribonucleotides which comprises a second RNA sequence of at least 48 contiguous ribonucleotides,
wherein the first RNA sequence hybridises to the second RNA sequence by basepairing between at least 42 ribonucleotides of the at least 44 contiguous ribonucleotides of the first RNA sequence and at least 42 ribonucleotides of the at least 48 contiguous ribonucleotides of the second RNA sequence, forming at least a part within the double-stranded RNA region,
wherein 4, 5 or 6 ribonucleotides of the at least 48 contiguous ribonucleotides of the second RNA sequence are non-basepaired and 0, 1 or 2, respectively, ribonucleotides of the at least 44 contiguous ribonucleotides of the first RNA sequence are non-basepaired in the part of the double-stranded RNA region, forming bulges in the part of the double-stranded RNA region,
wherein each of the bulges is immediately flanked by ribonucleotides which are basepaired in the part of the double-stranded RNA region,
wherein the second RNA sequence is at least 80% identical to a sequence of at least 48 contiguous ribonucleotides which is fully complementary to a first region of a target RNA molecule in an insect cell,
wherein cleavage of the precursor RNA molecule in the insect cell by one or more Dicers produces double-stranded product RNA molecules, each independently consisting of a sense RNA sequence of 21 contiguous ribonucleotides from the first RNA sequence and an antisense RNA sequence of 23 contiguous ribonucleotides from the second RNA sequence,
wherein at least some of the double-stranded RNA molecules have non-overlapping antisense RNA sequences,
wherein the antisense RNA sequence of the double-stranded product RNA molecules each comprise at least one of the bulges,
wherein ribonucleotides 20 and 21 of the sense RNA sequence in each of the double-stranded product RNA molecules form a 2 ribonucleotide unpaired 3′ overhang,
wherein ribonucleotides 22 and 23 of the antisense RNA sequence in each of the double-stranded product RNA molecules form a 2-ribonucleotide unpaired 3′ overhang, and
wherein either (iii) at least some of the antisense RNA sequences in the double-stranded product RNA molecules reduce the expression and/or activity of the target RNA molecule in the insect cell, or
(iv) the antisense RNA sequences in the double-stranded product RNA molecules are each, independently, at least 80% identical in sequence to a corresponding region of the complement of the target RNA molecule, or
(v) both (iii) and (iv).

45. A precursor RNA molecule comprising at least one double-stranded RNA region, wherein the double-stranded RNA region comprises:
(a) a RNA first strand of at least 46 contiguous ribonucleotides which comprises a first RNA sequence of at least 44 contiguous ribonucleotides, and
(b) a second RNA strand of at least 52 contiguous ribonucleotides which comprises a second RNA sequence of at least 50 contiguous ribonucleotides,
wherein the first RNA sequence hybridises to the second RNA sequence by basepairing between at least 42 ribonucleotides of the at least 44 contiguous ribonucleotides of the first RNA sequence and at least 42 ribonucleotides of the at least 50 contiguous ribonucleotides of the second RNA sequence, forming at least a part within the double-stranded RNA region,
wherein 6, 7 or 8 ribonucleotides of the at least 50 contiguous ribonucleotides of the second RNA sequence are non-basepaired and 0, 1 or 2, respectively, ribonucleotides of the at least 44 contiguous ribonucleotides of the first RNA sequence are non-basepaired in the part of the double-stranded RNA region, forming bulges in the part of the double-stranded RNA region,
wherein each of the bulges is immediately flanked by ribonucleotides which are basepaired in the part of the double-stranded RNA region,
wherein the second RNA sequence is at least 80% identical to a sequence of at least 50 contiguous ribonucleotides which is fully complementary to a first region of a target RNA molecule in an insect cell,
wherein cleavage of the precursor RNA molecule in the insect cell by one or more Dicers produces double-stranded product RNA molecules, each independently consisting of a sense RNA sequence of 21 contiguous ribonucleotides from the first RNA sequence and an antisense RNA sequence of 24 contiguous ribonucleotides from the second RNA sequence,
wherein at least some of the double-stranded RNA molecules have non-overlapping antisense RNA sequences,
wherein the antisense RNA sequence of the double-stranded product RNA molecules each comprise at least one of the bulges,
wherein ribonucleotides 20 and 21 of the sense RNA sequence in each of the double-stranded product RNA molecules form a 2-ribonucleotide unpaired 3′ overhang,
wherein ribonucleotides 23 and 24 of the antisense RNA sequence in each of the double-stranded product RNA molecules form a 2-ribonucleotide unpaired 3′ overhang, and
wherein either (iii) at least some of the antisense RNA sequences in the double-stranded product RNA molecules reduce the expression and/or activity of the target RNA molecule in the insect cell, or
(iv) the antisense RNA sequences in the double-stranded product RNA molecules are each, independently, at least 80% identical in sequence to a corresponding region of the complement of the target RNA molecule, or
(v) both (iii) and (iv).

46. The precursor RNA molecule of claim 43, wherein 2 ribonucleotides of the 46 contiguous ribonucleotides of the second RNA sequence are non-basepaired and all of the 44 contiguous ribonucleotides of the first RNA sequence are basepaired in the part of the double-stranded RNA region, the 2 ribonucleotides forming single-ribonucleotide bulges in the part of the double-stranded RNA region.

47. The precursor RNA molecule of claim 46, wherein the 2 ribonucleotides forming bulges are spaced apart by 16-26 contiguous basepairs in the part of the double-stranded RNA region.

48. The precursor RNA molecule of claim 43, wherein the first RNA sequence is shorter than the second RNA sequence, preferably wherein the first RNA sequence is shorter than the second RNA sequence entirely because of the presence of non-basepaired ribonucleotides in the second RNA sequence that bulge from the double-stranded RNA region, more preferably wherein the first RNA sequence has a length which is between 94% and 97% of the length of the second RNA sequence, or the length of the first RNA sequence is about 21/22 of the length of the second RNA sequence, calculated as a fraction.

49. The precursor RNA molecule of claim 43, wherein between 10% and 35% of the ribonucleotides of the first RNA sequence and the second RNA sequence, in total, are basepaired in G:U basepairs.

50. The precursor RNA molecule of claim 43, comprising a linking RNA sequence, wherein the linking RNA sequence links either the 3′ end of the first RNA strand to 5′ end of the second RNA strand, or 5′ end of the first RNA strand to the 3′ end of the second RNA strand.

51. The precursor RNA molecule of claim 43, wherein one or more of the following apply:
(i) the precursor RNA molecule comprises a linking RNA sequence which comprises a single-stranded RNA sequence of at least 44, or at least 50 or at least 100, contiguous ribonucleotides which is identical to a region of either the first RNA sequence or the second RNA sequence of the double-stranded RNA region,
(ii) the percentage of G ribonucleotides in the first RNA sequence is increased by A to G substitutions, relative to a corresponding sequence in the region of the target RNA molecule, and/or the percentage of G ribonucleotides in the second RNA sequence is increased by A to G substitutions, relative to a corresponding sequence in the complement of the region of the target RNA molecule,
(iii) the percentage of U ribonucleotides in the first RNA sequence is increased by C to U substitutions, relative to a corresponding sequence in the region of the target RNA molecule, and/or the percentage of U ribonucleotides in the second RNA sequence is increased by C to U substitutions, relative to a corresponding sequence in the complement of the region of the target RNA molecule,
(iv) at least some of the antisense RNA sequences in the double-stranded product RNA molecules produced from the precursor RNA molecule basepair along the full length of the antisense RNA sequences to the region of the target RNA molecule, preferably basepair along the full length by canonical basepairs,
(v) at least some of the antisense RNA sequences in the double-stranded product RNA molecules produced from the precursor RNA molecule reduce the expression and/or activity of the target RNA molecule in the insect cell or an insect, or the antisense RNA sequences in the double-stranded product RNA molecules produced from the precursor RNA molecule reduce the expression and/or activity of multiple, different target RNA molecules in the insect cell or insect, wherein the different target RNA molecules are unrelated in sequence, and
(vi) the reduction in expression and/or activity of the target RNA molecule(s) in the insect cell or insect results in death of the cell or larvae of the insect, or the mortality rate is increased relative to the use of antisense RNA sequences produced from a corresponding precursor RNA molecule with only canonical basepairing.

52. The precursor RNA molecule of claim 43, wherein all of the ribonucleotides of the antisense RNA sequences are capable of basepairing to ribonucleotides in the region of the target RNA molecule.

53. A double-stranded RNA molecule produced from the precursor RNA molecule of claim 43, or a population of such double-stranded RNA molecules, each double-stranded RNA molecule consisting of a sense RNA sequence of 21 contiguous ribonucleotides and an antisense RNA sequence of 22 contiguous ribonucleotides and comprising one or two or three bulged ribonucleotides,
wherein 18 or 19 of ribonucleotides 1 to 19 of the sense RNA sequence each basepair with a ribonucleotide of ribonucleotides 1 to 20 of the antisense RNA sequence,
wherein each of the one or two or three bulged ribonucleotides is immediately flanked by ribonucleotides which are basepaired in the double-stranded RNA molecule,
wherein ribonucleotides 20 and 21 of the sense RNA sequence form a 2-ribonucleotide unpaired 3′ overhang and ribonucleotides 21 and 22 of the antisense RNA sequence form a 2-ribonucleotide unpaired 3′ overhang,
wherein ribonucleotides 1 and 2 of the sense RNA sequence basepair with ribonucleotides 19 and 20, respectively, of the antisense RNA sequence, and
wherein ribonucleotides 18 and 19 of the sense RNA sequence basepair with ribonucleotides 1 and 2, respectively, of the antisense RNA sequence.

54. A double-stranded RNA molecule produced from the precursor RNA molecule of claim 44, or a population of such double-stranded RNA molecules, each double-stranded RNA molecule consisting of a sense RNA sequence of 21 contiguous ribonucleotides and an antisense RNA sequence of 23 contiguous ribonucleotides and comprising one or two or three or four bulged ribonucleotides,
wherein 18 or 19 of ribonucleotides 1 to 19 of the sense RNA sequence each basepair with a ribonucleotide of ribonucleotides 1 to 21 of the antisense RNA sequence,
wherein each of the one or two or three or four bulged ribonucleotides is immediately flanked by ribonucleotides which are basepaired in the double-stranded RNA molecule,
wherein ribonucleotides 20 and 21 of the sense RNA sequence form a 2-ribonucleotide unpaired 3′ overhang and ribonucleotides 22 and 23 of the antisense RNA sequence form a 2-ribonucleotide unpaired 3′ overhang,
wherein ribonucleotides 1 and 2 of the sense RNA sequence basepair with ribonucleotides 20 and 21, respectively, of the antisense RNA sequence, and
wherein ribonucleotides 18 and 19 of the sense RNA sequence basepair with ribonucleotides 1 and 2, respectively, of the antisense RNA sequence.

55. A double-stranded RNA molecule produced from the precursor RNA molecule of claim 45, or a population of such double-stranded RNA molecules, each double-stranded RNA molecule consisting of a sense RNA sequence of 21 contiguous ribonucleotides and an antisense RNA sequence of 24 contiguous ribonucleotides and comprising one or two or three or four or five bulged ribonucleotides,
wherein 18 or 19 of ribonucleotides 1 to 19 of the sense RNA sequence each basepair with a ribonucleotide of ribonucleotides 1 to 22 of the antisense RNA sequence,
wherein each of the one or two or three or four or five bulged ribonucleotides is immediately flanked by ribonucleotides which are basepaired in the double-stranded RNA molecule,
wherein ribonucleotides 20 and 21 of the sense RNA sequence form a 2-ribonucleotide unpaired 3′ overhang and ribonucleotides 23 and 24 of the antisense RNA sequence form a 2-ribonucleotide unpaired 3′ overhang,
wherein ribonucleotides 1 and 2 of the sense RNA sequence basepair with ribonucleotides 21 and 22, respectively, of the antisense RNA sequence, and
wherein ribonucleotides 18 and 19 of the sense RNA sequence basepair with ribonucleotides 1 and 2, respectively, of the antisense RNA sequence.

56. An isolated and/or exogenous polynucleotide, or a vector comprising the polynucleotide, encoding the precursor RNA molecule of claim 43, optionally wherein the polynucleotide is operably linked to a promoter capable of directing expression of the precursor RNA molecule in a host cell, preferably a plant cell, fungal cell or other microbial cell, or in vitro, and optionally comprising a polyadenylation region/transcription terminator or a transcription termination sequence.

57. A host cell, such as a eukaryotic cell, comprising one or more or all of the precursor RNA molecule of claim 43, a double-stranded RNA molecule produced therefrom, or a population of double-stranded RNA molecules produced therefrom, wherein the eukaryotic cell is preferably a plant cell, a yeast cell or an insect cell.

58. An extract of a cell, or a composition, wherein the extract or composition comprises one or more or all of the precursor RNA molecule of claim 43, a double-stranded RNA molecule produced therefrom, a population of double-stranded RNA molecules produced therefrom, or a polynucleotide or a vector encoding the precursor RNA molecule.

59. A method of identifying a double-stranded RNA molecule, or a precursor RNA molecule, for reducing the amount and/or activity of a target RNA molecule of interest in an insect cell or insect, the method comprising
i) producing the precursor RNA molecule of claim 43, or a population of multiple, different precursor RNA molecules, and/or a double-stranded RNA molecule produced therefrom, or a population of double-stranded RNA molecules produced from the precursor RNA molecule,
ii) determining the ability of the precursor RNA molecule or the double-stranded RNA molecule, or members of the population of multiple, different precursor RNA molecules, or the population of double-stranded RNA molecules, to reduce the amount and/or activity of the target RNA molecule of interest in the insect cell or insect, optionally wherein step i) comprises introducing the precursor RNA molecule into the insect cell or insect, preferably by topical application such as soaking, dusting, spraying or applying a composition comprising the precursor RNA molecule to the insect cell or insect, wherein the precursor RNA molecule is cleaved in the insect cell or insect by a Dicer to produce the double-stranded RNA molecule or the population of double-stranded RNA molecules.

60. A non-human organism, or a part thereof, preferably a plant or part thereof or a fungus, comprising one or more or all of the precursor RNA molecule of claim 43, a double-stranded RNA molecule produced therefrom, a population of double-stranded RNA molecules produced therefrom, a polynucleotide or vector encoding the precursor RNA molecule, wherein the non-human organism is preferably a transgenic plant, being transgenic for a polynucleotide encoding the precursor RNA molecule, preferably wherein the polynucleotide is stably integrated into the genome of the plant or part thereof.

61. A method of producing a cell, or a non-human organism comprising the cell, the method comprising introducing into a cell one or more or all of the precursor RNA molecule of claim 43, a double-stranded RNA molecule produced therefrom, a population of double-stranded RNA molecules produced therefrom, or a polynucleotide or vector encoding the precursor RNA molecule, optionally generating the non-human organism from the cell.

62. A method of producing one or more or all of the precursor RNA molecule of claim 43, a double-stranded RNA molecule produced therefrom, or a population of double-stranded RNA molecules produced therefrom, the method comprising expressing a polynucleotide or vector encoding the precursor RNA molecule in a host cell or cell-free expression system.

63. A method for increasing the number of double-stranded RNA molecules in an insect cell or insect, or for increasing the amount of small interfering RNAs (sRNAs) of 22, 23 or 24 ribonucleotides in length in an insect cell or insect, or of increasing the ratio of the amount of sRNAs of 22, 23 or 24 ribonucleotides relative to the amount of sRNAs of 21 ribonucleotides in length, the method comprising expressing in the insect cell or insect a polynucleotide or a vector encoding the precursor RNA molecule of claim 43, or contacting the insect cell or insect with one or more of the precursor RNA molecule, a double-stranded RNA molecule produced therefrom, a population of double-stranded RNA molecules produced therefrom, or a cell comprising the precursor RNA molecule.

64. A method for reducing or down-regulating the level and/or activity of a target RNA molecule in an insect cell or insect, the method comprising delivering to the insect cell or insect one or more or all of the precursor RNA molecule of claim 43, a double-stranded RNA molecule produced therefrom, a population of double-stranded RNA molecules produced therefrom, a polynucleotide or a vector encoding the precursor RNA molecule, an extract comprising the precursor RNA molecule, a composition comprising the precursor RNA molecule, or a cell comprising the precursor RNA molecule.

65. A method for identifying a function or phenotype associated with a target RNA molecule in an insect cell or insect, the method comprising (i) delivering to the insect cell or insect, one or more or all of: the precursor RNA molecule of claim 43, a double-stranded RNA molecule produced therefrom, a population of double-stranded RNA molecules produced therefrom, a polynucleotide or vector encoding the precursor RNA molecule, an extract comprising the precursor RNA molecule, a composition comprising the precursor RNA molecule, or a cell comprising the precursor RNA molecule, and (ii) determining a function or phenotype of the insect cell or insect, or assaying the insect cell or insect, or a progeny insect cell or insect thereof, for a molecule associated with the function or a phenotype, thereby identifying the function or phenotype associated with a target RNA.

66. A method for identifying an RNA molecule that is capable of having an effect on an insect, the method comprising (i) delivering to the insect, one or more or all of the precursor RNA molecule of claim 43, a double-stranded RNA molecule produced therefrom, a population of double-stranded RNA molecules produced therefrom, a polynucleotide or vector encoding the precursor RNA molecule, an extract comprising the precursor RNA molecule, a composition comprising the precursor RNA molecule, or a cell comprising the precursor RNA molecule, and (ii) determining whether or not the precursor RNA molecule, double-stranded RNA molecule or population of different double-stranded RNA molecules has an effect on the inscet, and optionally (iii) if the precursor RNA molecule, double-stranded RNA molecule or population of different double-stranded RNA molecules has a desirable effect on the insect, selecting an RNA molecule based on results from step (ii), thereby identifying the RNA molecule.

67. A method of reducing or preventing damage caused by an insect to a human or non-human organism, or of controlling an insect, the method comprising delivering to the insect, or contacting the insect with, one or more or all of the precursor RNA molecule of claim 43, a double-stranded RNA molecule produced therefrom, a population of double-stranded RNA molecules produced therefrom, a polynucleotide or vector encoding the precursor RNA molecule, an extract comprising the precursor RNA molecule, a composition comprising the precursor RNA molecule, or a cell comprising the precursor RNA molecule, preferably wherein the precursor RNA molecule is produced in a plant or a yeast cell.

68. A method of treating a disease in an organism, the method comprising administering to an insect pest of the organism one or more or all of the precursor RNA molecule of claim 43, a double-stranded RNA molecule produced therefrom, a population of double-stranded RNA molecules produced therefrom, a polynucleotide or vector encoding the precursor RNA molecule, an extract comprising the precursor RNA molecule, a composition comprising the precursor RNA molecule, or a cell comprising the precursor RNA molecule.


SEQUENCE LISTING (285 sequences)

SEQ ID NO: 1 - DNA, 1812 bp, synthetic construct
  source 1..1812: mol_type = other DNA
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttcccgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 2 - DNA, 200 bp, synthetic construct
  source 1..200: mol_type = other DNA
tcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacg

SEQ ID NO: 3 - DNA, 200 bp, synthetic construct
  source 1..200: mol_type = other DNA
ttgtgttggtatttggttagtggtagtgaagggtgaatagtttttgattaattataaattgttttattttattggttttggttgttatgaagatgtggatttgtgtggtaaaggatttgataatgtgttgatggtgtatgattatgtattaatggattggattggggttaatttttattgtattttgtattatttttatg

SEQ ID NO: 4 - RNA, 463 nt, synthetic construct
  source 1..463: mol_type = other RNA
ggatcctcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgaagcttggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 5 - RNA, 457 nt, synthetic construct
  source 1..457: mol_type = other RNA
ctcgagttgtgttggtatttggttagtggtagtgaagggtgaatagtttttgattaattataaattgttttattttattggttttggttgttatgaagatgtggatttgtgtggtaaaggatttgataatgtgttgatggtgtatgattatgtattaatggattggattggggttaatttttattgtattttgtattatttttatgggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 6 - DNA, 295 bp, synthetic construct
  source 1..295: mol_type = other DNA
gtcgacgatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggatccagacgcgtggttacagtcttgcgcgacatgcgtcaccacggtgatatcgtccacccaggtgttcggcgtggtgtagagcattacgctgcgatcgtcgac

SEQ ID NO: 7 - protein, 174 aa, Helicoverpa armigera
MFNQTQFFVFLAVFTTSSVLGNNNDVKDGGAASGAHSDRLGLWFGPRLGKRSLRISTEDNRQAFFKLLEAADALKYYYDQLPYEMQADEPETRVTKKVIFTPKLGRSLAYDDKSFENVEFTPRLGRRLSDDMPATPADQEMYRQDPEQIDSRTKYFSXGKLQSETKHIKVIKYS

SEQ ID NO: 8 - DNA, 560 bp, synthetic construct
  source 1..560: mol_type = other DNA
aggagtatcattcgaattcgatcgttcattccaagatgttcaatcaaactcagttctttgttttccttgctgttttcactacgagcagtgttttagggaataacaatgatgttaaggatggtggcgcagcgagtggagctcacagcgaccgactaggcctttggttcggtccccggctaggcaagcgctctctcaggatatctaccgaagataacagacaagcattcttcaaactgcttgaagctgccgatgctttgaaatactattacgaccaactgccttatgagatgcaagctgatgaacctgaaaccagagtgaccaaaaaggtgatcttcacccccaagctaggcagaagcctcgcatacgatgacaagagcttcgaaaacgtggagttcactcctagactcggcaggagactgtccgatgatatgcctgccactcccgctgaccaggaaatgtatcgtcaagaccctgaacagattgacagcaggacaaagtacttctccccngggaagctacagtcagaaactaaacatattaaagtaataaaatattcataa

SEQ ID NO: 9 - DNA, 280 bp, Helicoverpa armigera
  source 1..280: mol_type = genomic DNA
atgttcaatcaaactcagttctttgttttccttgctgttttcactacgagcagtgttttagggaataacaatgatgttaaggatggtggcgcagcgagtggagctcacagcgaccgactaggcctttggttcggtccccggctaggcaagcgctctctcaggatatctaccgaagataacagacaagcattcttcaaactgcttgaagctgccgatgctttgaaatactattacgaccaactgccttatgagatgcaagctgatgaacctgaaaccagag

SEQ ID NO: 10 - DNA, 280 bp, synthetic construct
  source 1..280: mol_type = other DNA
atgtttaattaaatttagttttttgttttttttgttgtttttattatgagtagtgttttagggaataataatgatgttaaggatggtggtgtagtgagtggagtttatagtgattgattaggtttttggtttggtttttggttaggtaagtgtttttttaggatatttattgaagataatagataagtattttttaaattgtttgaagttgttgatgttttgaaatattattatgattaattgttttatgagatgtaagttgatgaatttgaaattagag

SEQ ID NO: 11 - DNA, 754 bp, synthetic construct
  source 1..754: mol_type = other DNA
ggatcctcgagtaatacgactcactatagggctctggtttcaggttcatcagcttgcatctcataaggcagttggtcgtaatagtatttcaaagcatcggcagcttcaagcagtttgaagaatgcttgtctgttatcttcggtagatatcctgagagagcgcttgcctagccggggaccgaaccaaaggcctagtcggtcgctgtgagctccactcgctgcgccaccatccttaacatcattgttattccctaaaacactgctcgtagtgaaaacagcaaggaaaacaaagaactgagtttgattgaacattgaccaaaaaggtgatcttcacccccaagctaggcagaagcctcgcatacgatgacaagagcttcgaaaacgtggagttcactcctagactcggcaggagactgtccgatgatatgcctgccactcccgctgaccaggaaatgtatcgtcatgttcaatcaaactcagttctttgttttccttgctgttttcactacgagcagtgttttagggaataacaatgatgttaaggatggtggcgcagcgagtggagctcacagcgaccgactaggcctttggttcggtccccggctaggcaagcgctctctcaggatatctaccgaagataacagacaagcattcttcaaactgcttgaagctgccgatgctttgaaatactattacgaccaactgccttatgagatgcaagctgatgaacctgaaaccagagcccgggaagcttc

SEQ ID NO: 12 - DNA, 754 bp, synthetic construct
  source 1..754: mol_type = other DNA
ggatcctcgagtaatacgactcactatagggctctggtttcaggttcatcagcttgcatctcataaggcagttggtcgtaatagtatttcaaagcatcggcagcttcaagcagtttgaagaatgcttgtctgttatcttcggtagatatcctgagagagcgcttgcctagccggggaccgaaccaaaggcctagtcggtcgctgtgagctccactcgctgcgccaccatccttaacatcattgttattccctaaaacactgctcgtagtgaaaacagcaaggaaaacaaagaactgagtttgattgaacattgaccaaaaaggtgatcttcacccccaagctaggcagaagcctcgcatacgatgacaagagcttcgaaaacgtggagttcactcctagactcggcaggagactgtccgatgatatgcctgccactcccgctgaccaggaaatgtatcgtcatgtttaattaaatttagttttttgttttttttgttgtttttattatgagtagtgttttagggaataataatgatgttaaggatggtggtgtagtgagtggagtttatagtgattgattaggtttttggtttggtttttggttaggtaagtgtttttttaggatatttattgaagataatagataagtattttttaaattgtttgaagttgttgatgttttgaaatattattatgattaattgttttatgagatgtaagttgatgaatttgaaattagagcccgggaagcttc

SEQ ID NO: 13 - DNA, 333 bp, synthetic construct
  source 1..333: mol_type = other DNA
atggaattgaacgaaggcgcaatgacaaacgtcgaactccaactggctcgtatggtggaggcggcggctgcggcagcagcttctcacaagcagaatgcagcggcagcaggtcacaaaagtcccagcgagagggcgcgttcaaatctcaggctgttccgcttcctaccgttctatcaagtagatggttcggaactgatagagatgtaccaccatgtgaacatggtgggattgtccgagtctgaggccccttgttttacgttgccagcggaagaagaccatgatttcgacgatacggattggttcgctggtaacgagtgggcggaagggtcgttc

SEQ ID NO: 14 - protein, 111 aa, synthetic construct
MELNEGAMTNVELQLARMVEAKRQRRRSHKQNRRERGHKSPSERARSNLRLFRFLPFYQVDGSELIEMYHHVNMVGLSESEAPCFTLPAEEDHDFDDTDWFAGNEWAEGSF

SEQ ID NO: 15 - protein, 646 aa, Helicoverpa armigera
MPVRTTTFEMISNTKIVFTKLLLCCFVSGAVARSWANHHDTTTSTTQTTPTTSPVPKNFHNDPLIVETKSGLVKGYAKTVMGREVHIFTGIPFAKPPLGPLRFRKPVPIDPWHGVLEATAMPNSCYQERYEYFPGFEGEEMWNPNTNISEDCLYLNIWVPQHLRVRHHQDKPLAERPKVPILVWIYGGGYMSGTATLNLYKADIMASSSDVIVASMQYRVGAFGFLYLNKYFSPGSEEAPGNMGLWDQQLAIRWIKDNARAFGGDPELITLFGESAGGGSVSLHMLSPEMKGLFKRGILQSGTLNAPWSWMTGERAQDIGKVLVDDCNCNSSLLAADPSLVMDCMRGVDAKTISVQQWNSYTGILGFPSAPTVDGVFLPKDPDTMMKEGNFHNTEVLLGSNQDEGTYFLLYDFLDYFEKDGPSFLQREKFLEIVDTIFKDFSKIKREAIVFQYTDWEEITDGYLNQKMIADVVGDYFFVCPTNYFAEVLADSGVDVYYYYFTHRTSTSLGEWMGVMHGDEMEYVFGHPLNMSLQYHTRERDLAAHIMQSFTRFALTGKPHKPDEKWPLYSRTSPHYYTYTADGASGPAGPRGPRASACAFWNDFLNKLNELEHMPCDGAVTGLYSSVAGTTLPIVLLTTLATTVAL

SEQ ID NO: 16 - DNA, 2551 bp, synthetic construct
  source 1..2551: mol_type = other DNA
acgcggggagtctactctgtcacgtcgatggagacgcatcgcggtcggcctctctctcaatattattactttattaccatacaaaacgtgagtgagtgtctgtgaacttatttcaacttgagtgacggttgctccggaccatacagcgagctgacagttcagccaaggtcatcgatcagcaatttgacaccagttgtgtttatatactatatgtacgaccacaacagaataccgtgtgcccctagaaatgtgttttaacaagtatctcattaagctggcggattacttcagatcagtttctgatacgtctcgttaatgcccgtgcgaacaacaacattcgaaatgatcagcaacacgaagatcgtgttcaccaagctcctgctgtgctgcttcgtgtcaggcgccgtcgcgaggtcgtgggccaaccatcacgacaccaccacgtcaaccacgcagaccacccccaccactagtcctgtgcctaagaacttccataatgatccactcatcgtcgaaactaaaagtggcctcgtcaaaggctacgctaagacagttatgggcagggaagtacacatcttcaccggtattccgtttgcgaagccccctctaggaccgttaagattccgcaaaccggtacccatcgacccgtggcatggagtacttgaagccactgcgatgccaaatagctgttatcaagaacggtatgagtacttccccggttttgagggagaggaaatgtggaatccaaacacaaatatatcagaggactgtctctatctaaacatttgggttccccagcacttacgagtccgtcatcatcaagataagcctcttgcggagaggcccaaagtgccgatactagtgtggatttacggtggcggctatatgagtggcacggcaacactcaacctatataaagcagacataatggcttcttccagtgatgtaatagtagcatctatgcaatatagggttggggcgttcggatttttatacttgaataaatatttctcgccgggcagtgaagaggcgccgggaaatatgggcttatgggatcaacaactcgctattcgttggattaaagataatgctcgtgcttttggtggtgacccagaattgataactttgtttggagagtcggcaggcgggggaagcgtgagtttgcatatgctttcaccagagatgaagggattattcaaacgaggcatcttgcaatctggaacgttaaacgctccatggagttggatgacaggagagagagcacaagacataggaaaagtgttagtagatgactgtaattgcaatagcagcctactagctgccgatccgagcttagtgatggactgtatgcgcggagtcgatgcaaaaactatatcagtacagcagtggaattcctatactggcatattgggattcccgtcagctcccacggttgacggcgtgtttttgcccaaagacccggacacaatgatgaaagaaggcaatttccataataccgaggtgcttcttggaagtaatcaagacgaaggaacctatttcttactatacgatttccttgactacttcgagaaagacgggcccagcttcttgcagcgggagaaatttctagaaattgtcgacaccatattcaaggatttctccaaaataaagagggaagctatcgtattccaatatacggactgggaggaaattaccgatggctatctgaaccagaaaatgatagctgacgtggtgggcgactatttcttcgtgtgccctactaactacttcgcagaagtgctggccgattcaggagttgatgtttactactattacttcacacatcgcaccagcacaagtctctggggtgaatggatgggcgtgatgcacggcgatgagatggagtacgtcttcgggcacccgctgaacatgtcccttcaataccacaccagggagagggaccttgctgcacatatcatgcagtcttttacgagattcgctttgactggcaaacctcacaagccggacgagaaatggccgctgtactctcgcacgtcgccccactactacacttacaccgcggacggggctagcgggccagccggaccgcgcgggcctagggcctccgcttgcgctttctggaatgatttccttaacaagctgaatgagctggagcacatgccatgtgacggggccgtgaccggcctttacagcagcgtcgccggcaccaccctgccgatagtgctgctgaccacgctcgccaccaccgtcgcactctaagcgaccgacattcactcgtagcaaacagtagacaaattatttatacaaactagaaaaataccgatgcaaataaatgttttcacttcatctataaccataaagtcaaaaatcttatctgaaatgaactttatgattgtcaatgaagatctagagtacgcaaatcgtatttcgaattttaatatcggctactaaagtactagaagtaaattgttgtattaattaaatatattttatcacaatcttacatatcaagcgtgtggaaagaaaacgggacagtgacaaggaacgttac

SEQ ID NO: 17 - DNA, 494 bp, synthetic construct
  source 1..494: mol_type = other DNA
acggcaacactcaacctatataaagcagacataatggcttcttccagtgatgtaatagtagcatctatgcaatatagggttggggcgttcggatttttatacttgaataaatatttctcgccgggcagtgaagaggcgccgggaaatatgggcttatgggatcaacaactcgctattcgttggattaaagataatgctcgtgcttttggtggtgacccagaattgataactttgtttggagagtcggcaggcgggggaagcgtgagtttgcatatgctttcaccagagatgaagggattattcaaacgaggcatcttgcaatctggaacgttaaacgctccatggagttggatgacaggagagagagcacaagacataggaaaagtgttagtagatgactgtaattgcaatagcagcctactagctgccgatccgagcttagtgatggactgtatgcgcggagtcgatgcaaaaactatatcagtacagcagtg

SEQ ID NO: 18 - DNA, 494 bp, synthetic construct
  source 1..494: mol_type = other DNA
atggtaatatttaatttatataaagtagatataatggttttttttagtgatgtaatagtagtatttatgtaatatagggttggggtgtttggatttttatatttgaataaatattttttgttgggtagtgaagaggtgttgggaaatatgggtttatgggattaataatttgttatttgttggattaaagataatgtttgtgtttttggtggtgatttagaattgataattttgtttggagagttggtaggtgggggaagtgtgagtttgtatatgtttttattagagatgaagggattatttaaatgaggtattttgtaatttggaatgttaaatgttttatggagttggatgataggagagagagtataagatataggaaaagtgttagtagatgattgtaattgtaatagtagtttattagttgttgatttgagtttagtgatggattgtatgtgtggagttgatgtaaaaattatattagtatagtagtg

SEQ ID NO: 19 - DNA, 1294 bp, synthetic construct
  source 1..1294: mol_type = other DNA
ggatcctcgagtaatacgactcactatagggcactgctgtactgatatagtttttgcatcgactccgcgcatacagtccatcactaagctcggatcggcagctagtaggctgctattgcaattacagtcatctactaacacttttcctatgtcttgtgctctctctcctgtcatccaactccatggagcgtttaacgttccagattgcaagatgcctcgtttgaataatcccttcatctctggtgaaagcatatgcaaactcacgcttcccccgcctgccgactctccaaacaaagttatcaattctgggtcaccaccaaaagcacgagcattatctttaatccaacgaatagcgagttgttgatcccataagcccatatttcccggcgcctcttcactgcccggcgagaaatatttattcaagtataaaaatccgaacgccccaaccctatattgcatagatgctactattacatcactggaagaagccattatgtctgctttatataggttgagtgttgccgtgccactcatatagccgccaccgtaaatccacactagtatcggcactttgggcctctccgcaagaggcttatcttgatgatgacggactcgtaagtgctggggaacccaaatgtttagatagagacagtcctctgatatatttgtgtttggattccacatttcctctccctcaaaaccggggaagtactcataccgttcttgataacagctatttggcatcgcagtggcttcaagtactccatgccacgggtcgatgggtaccacggcaacactcaacctatataaagcagacataatggcttcttccagtgatgtaatagtagcatctatgcaatatagggttggggcgttcggatttttatacttgaataaatatttctcgccgggcagtgaagaggcgccgggaaatatgggcttatgggatcaacaactcgctattcgttggattaaagataatgctcgtgcttttggtggtgacccagaattgataactttgtttggagagtcggcaggcgggggaagcgtgagtttgcatatgctttcaccagagatgaagggattattcaaacgaggcatcttgcaatctggaacgttaaacgctccatggagttggatgacaggagagagagcacaagacataggaaaagtgttagtagatgactgtaattgcaatagcagcctactagctgccgatccgagcttagtgatggactgtatgcgcggagtcgatgcaaaaactatatcagtacagcagtgcccgggaagcttc

SEQ ID NO: 20 - DNA, 1294 bp, synthetic construct
  source 1..1294: mol_type = other DNA
ggatcctcgagtaatacgactcactatagggcactgctgtactgatatagtttttgcatcgactccgcgcatacagtccatcactaagctcggatcggcagctagtaggctgctattgcaattacagtcatctactaacacttttcctatgtcttgtgctctctctcctgtcatccaactccatggagcgtttaacgttccagattgcaagatgcctcgtttgaataatcccttcatctctggtgaaagcatatgcaaactcacgcttcccccgcctgccgactctccaaacaaagttatcaattctgggtcaccaccaaaagcacgagcattatctttaatccaacgaatagcgagttgttgatcccataagcccatatttcccggcgcctcttcactgcccggcgagaaatatttattcaagtataaaaatccgaacgccccaaccctatattgcatagatgctactattacatcactggaagaagccattatgtctgctttatataggttgagtgttgccgtgccactcatatagccgccaccgtaaatccacactagtatcggcactttgggcctctccgcaagaggcttatcttgatgatgacggactcgtaagtgctggggaacccaaatgtttagatagagacagtcctctgatatatttgtgtttggattccacatttcctctccctcaaaaccggggaagtactcataccgttcttgataacagctatttggcatcgcagtggcttcaagtactccatgccacgggtcgatgggtaccatggtaatatttaatttatataaagtagatataatggttttttttagtgatgtaatagtagtatttatgtaatatagggttggggtgtttggatttttatatttgaataaatattttttgttgggtagtgaagaggtgttgggaaatatgggtttatgggattaataatttgttatttgttggattaaagataatgtttgtgtttttggtggtgatttagaattgataattttgtttggagagttggtaggtgggggaagtgtgagtttgtatatgtttttattagagatgaagggattatttaaatgaggtattttgtaatttggaatgttaaatgttttatggagttggatgataggagagagagtataagatataggaaaagtgttagtagatgattgtaattgtaatagtagtttattagttgttgatttgagtttagtgatggattgtatgtgtggagttgatgtaaaaattatattagtatagtagtgcccgggaagcttc

SEQ ID NO: 21 - protein, 584 aa, Helicoverpa armigera
MSIESRLDSLVRGKSEVKAFFGGCPSALVDTGACDTLADMRRRWYNNGGFQTLRMLEESSSEVTSSSALGLPPAMVMSPESLASPEYGGLELWGYDDGITYSMAQSLGTCTMEQQQPQPQQQPQQTQPLPSMPLPMPPTTPKSENESMSSGREELSPASSVNGCSTDGEARRQKKGPAPRQQEELCLVCGDRASGYHYNALTCEGCKGFFRRSVTKNAVYICKFGHACEMDMYMRRKCQECRLKKCLAVGMRPECVVPENQCAMKRKEKKAQREKDKLPVSTTTVDDHMPPIMQCDPPPPEAARIHEVVPRFLNEKLMEQNRLKNVPPLTANQKSLIARLVWYQEGYEQPSEEDLKRVTQTWQSDEDDEDSDMPFRQITEMTILTVQLIVEFAKGLPGFAKISQSDQITLLKACSSEVMMLRVARRYDAATDSVLFANNQAYTRDNYRKAGMAYVIEDLLHFCRCMYSMMMDNVHYALLTAIVIFSDRPGLEQPLLVEEIQRYYLNTLRVYILNQNSASPRCAVIFGKILGILTEIRTLGMQNSNMCISLKLKNRKLPPFLEEIWDVADVSTTATPVVADSPAL

SEQ ID NO: 22 - DNA, 2407 bp, synthetic construct
  source 1..2407: mol_type = other DNA
tccgctgacgacggtcgcgcgcgcgcaacgtactcgtttttactgctcgaacaacgcgttaacccccgtctccacatcaccggacgaactcgtgggactcgcgtgctcttctcacctgttgctcggattgtgttgtgactagaaaaaagttgtcgccgctcgaacgagacttccgagtcctattggattgcacgaaagtcgagacagtggatagcgattcgtttcggtttgaacgttgcgtagacgagtggtgcatgtccatagagtcgcgtttagatagtttagtgcgaggaaaaagtgaagtgaaagccttcttcggaggatgtccctcggcgctcgtggataccggagcgtgtgacacgctcgcagacatgagacgccgctggtataacaacggaggattccagacgctgcgaatgctcgaggagagctcgtctgaggtgacgtcgtcttcagcactgggcctgccgccggctatggtgatgtccccggaatcgctcgcgtcgcccgagtacggcggcctggagctgtggggctacgacgatggcatcacttacagcatggcgcagtcgctgggcacctgcaccatggagcagcagcagccgcagccgcaacagcagccgcagcagacacagcccctaccttccatgccgttaccaatgccaccgacaacacctaaatcagaaaacgagtcaatgtcatcaggtcgtgaggagttgtctccagcttcaagtgtgaacggctgcagcacagatggcgaggcgaggcggcagaagaaaggcccagcgccgaggcagcaagaagagctatgtcttgtctgcggcgacagagcctccggatatcactacaacgcactcacatgtgaagggtgtaaaggtttcttcaggcggagtgtaaccaaaaatgcagtgtacatatgcaaattcggccatgcttgcgaaatggatatgtatatgcggagaaaatgtcaggagtgtcggttgaagaaatgtctcgcggtaggcatgaggcccgagtgcgtggtgccggagaaccagtgtgcaatgaaacggaaagagaaaaaggcgcagagggaaaaagacaaattgcccgtcagtacgacgacagtagacgatcacatgcctcccatcatgcaatgtgatcctccgcccccagaggccgctagaattcacgaggtggtgccgcggttcctcaatgagaagctcatggaacagaacagattgaagaacgtgccccccctcactgccaatcagaagtcgttgatcgcaaggctcgtgtggtaccaggaaggctacgaacaaccttccgaggaagacctgaagagggttacacagacctggcagtcggacgaggacgacgaggactcggatatgccgttccgtcagattaccgagatgacgattctcacagtgcagctcatcgtagaattcgctaaaggcctaccgggcttcgccaagatctcgcagtcagatcagatcacgttattaaaggcgtgctcaagtgaggtgatgatgttgcgagtggctcggcggtacgacgcggctaccgacagcgtgctgttcgccaacaaccaggcctacactcgcgacaactaccgcaaggcaggcatggcgtacgtcatcgaagacctgctgcacttctgtcggtgcatgtactccatgatgatggacaacgtgcactatgcgctgcttacagccattgtcatcttctcagaccggcctgggcttgagcaacccctgttggtggaggagatccagagatattacctgaacacgctacgggtgtacatcctgaaccagaacagcgcgtcgccccgctgcgccgtgatcttcggcaagatcctgggcatcctgacggagatccgcacgctgggcatgcagaactccaacatgtgcatctccctcaagctgaagaacaggaagctgccgccgttcctcgaggagatctgggacgtggcggacgtgtcgacgacggcgacgccggtggtggcggactcgccggcgctctagcccccgcgccgcccgcccggccgcgctcacgtctagcgcggcccctcaggagagaacgctcatagactggctagttttagtgaagtgcacggacactgacgtcggacgtgatcaacctatttataaggactgcgaattttaccacttaagagggcacacccgtacccgatttcgtacgtattcggtgaccgacgacgatgctgtgcgagagattagtgaatatatgtgttgttgaacgtttggagaatatatattggtgttgatcgttacgggcccgcgccgtcgccggtcggccggctgaccggcggccgccgcggctccgagtttatttcgtttacgactggatactgagttggtcactcggatacgactgtatgataagacttcgttcgataagt

SEQ ID NO: 23 - DNA, 300 bp, synthetic construct
  source 1..300: mol_type = other DNA
tcactgccaatcagaagtcgttgatcgcaaggctcgtgtggtaccaggaaggctacgaacaaccttccgaggaagacctgaagagggttacacagacctggcagtcggacgaggacgacgaggactcggatatgccgttccgtcagattaccgagatgacgattctcacagtgcagctcatcgtagaattcgctaaaggcctaccgggcttcgccaagatctcgcagtcagatcagatcacgttattaaaggcgtgctcaagtgaggtgatgatgttgcgagtggctcggcggtacgacg

SEQ ID NO: 24 - DNA, 300 bp, synthetic construct
  source 1..300: mol_type = other DNA
ttattgttaattagaagttgttgattgtaaggtttgtgtggtattaggaaggttatgaataattttttgaggaagatttgaagagggttatatagatttggtagttggatgaggatgatgaggatttggatatgttgttttgttagattattgagatgatgatttttatagtgtagtttattgtagaatttgttaaaggtttattgggttttgttaagattttgtagttagattagattatgttattaaaggtgtgtttaagtgaggtgatgatgttgtgagtggtttggtggtatgatg

SEQ ID NO: 25 - DNA, 794 bp, synthetic construct
  source 1..794: mol_type = other DNA
aactcgagtaatacgactcactatagggcgtcgtaccgccgagccactcgcaacatcatcacctcacttgagcacgcctttaataacgtgatctgatctgactgcgagatcttggcgaagcccggtaggcctttagcgaattctacgatgagctgcactgtgagaatcgtcatctcggtaatctgacggaacggcatatccgagtcctcgtcgtcctcgtccgactgccaggtctgtgtaaccctcttcaggtcttcctcggaaggttgttcgtagccttcctggtaccacacgagccttgcgatcaacgacttctgattggcagtgagcagcgcatagtgcacgttgtccatcatcatggagtacatgcaccgacagaagtgcagcaggtcttcgatgacgtacgccatgcctgccttgcggtagttgtcgcgagtgtaggcctggttgttggcgaacagcacgctgtcggtagccgtcactgccaatcagaagtcgttgatcgcaaggctcgtgtggtaccaggaaggctacgaacaaccttccgaggaagacctgaagagggttacacagacctggcagtcggacgaggacgacgaggactcggatatgccgttccgtcagattaccgagatgacgattctcacagtgcagctcatcgtagaattcgctaaaggcctaccgggcttcgccaagatctcgcagtcagatcagatcacgttattaaaggcgtgctcaagtgaggtgatgatgttgcgagtggctcggcggtacgacgcccgggcgtctagaga

SEQ ID NO: 26 - DNA, 794 bp, synthetic construct
  source 1..794: mol_type = other DNA
aactcgagtaatacgactcactatagggcgtcgtaccgccgagccactcgcaacatcatcacctcacttgagcacgcctttaataacgtgatctgatctgactgcgagatcttggcgaagcccggtaggcctttagcgaattctacgatgagctgcactgtgagaatcgtcatctcggtaatctgacggaacggcatatccgagtcctcgtcgtcctcgtccgactgccaggtctgtgtaaccctcttcaggtcttcctcggaaggttgttcgtagccttcctggtaccacacgagccttgcgatcaacgacttctgattggcagtgagcagcgcatagtgcacgttgtccatcatcatggagtacatgcaccgacagaagtgcagcaggtcttcgatgacgtacgccatgcctgccttgcggtagttgtcgcgagtgtaggcctggttgttggcgaacagcacgctgtcggtagccgttattgttaattagaagttgttgattgtaaggtttgtgtggtattaggaaggttatgaataattttttgaggaagatttgaagagggttatatagatttggtagttggatgaggatgatgaggatttggatatgttgttttgttagattattgagatgatgatttttatagtgtagtttattgtagaatttgttaaaggtttattgggttttgttaagattttgtagttagattagattatgttattaaaggtgtgtttaagtgaggtgatgatgttgtgagtggtttggtggtatgatgcccgggcgtctagaga

SEQ ID NO: 27 - protein, 950 aa, Helicoverpa armigera
MPLRLDIKRKLTARSDRVKCVDQHPSEPWLLCSLYNGDVNIWNYETHTQIKRFEVCDLPVRAAKFVPRKNWVITGSDDMQIRVFNYNTLERVHAFEAHSDYVRCIAIHPTQPYILTSSDDLLIKLWNWERNWACQQVFEGHTHYVMQIVINPKDNNTFASASLDTTVKVWQLGSSISNFTLEGHEKGVNCVDYYHGGDKPYLISGADDRLVKIWDYQNKTCVQTLESHTQNVSAVSFHPELPILLTGSEDGTIRIWHAGTYRLESSLNYGFERVWTISSMHGSNNVAIGYDEGTIMIKVGREEPAISMDVNGGKIIWAKHSEMQQVNLKALPEGTEIKDGERVPVMAKDMGSCEIYPQTIAHNPNGRFVVVCGDGEYIIYTAMALRNKAFGTAQEFVWAFDSSEYATLENSSTIKVFKNFKERKSFKPEFGAEGIYGGFMLGVKSISGVAFSFYDWENLELIRRIEIQPRHVYWSESGNLVCLATDDSYYVLKYNAAVVTRARETNTNITEDGIEDAFEVVGAVNEIVRTGLWVGDCFIYTNSVNRINYYVGGEIVTISHLDHTMYILGYVAKENRLYLNDKELNIVSYSLLLSVLEYQTAVMRADFETADRVLPTIPHEHRTRVAHFLEKQGFKQQALAVSTEPEHQFELALSLGELRRAKQLAEEAAVAEGAPSRSSSARWSRLGAAAAAAADTELTKACYQSAKDYSALLLFAASTGDKTLLRNVAQMSSEEGAENISFVAYFMLNDLDACLNLLIQRNKLPEAAFFARSYIPSKMSEVVKLWRESTSATNKKLGQSLADPEQYENLFPEYLQSLDLERFQREYGYEQSCLLANLPEDFKTCNIDRDLAAEKAEAEKNGFKLPPIVQTSQSTSAAVADISLTTSDCPTSDGSSSKAPEQVPQALPSAPKKRNDSLDIMDELEREIDDIILDSTADSVDLSDDADLMD

SEQ ID NO: 28 - DNA, 3057 bp, synthetic construct
  source 1..3057: mol_type = other DNA
aaaatagtgacacgtccacccaccctgtcaaacttccacagtttccctgttcctgtatgataaattttcatgttttaactattaaaaatgccgttacgcctggatatcaagaggaagttgacagcgaggtcggaccgcgtcaagtgcgtggaccagcacccgtcggagccatggctcctgtgctccttgtacaacggtgacgtgaacatatggaactatgagacacacacgcagatcaaacgcttcgaggtatgcgacctgcccgtgcgtgccgcgaagtttgtcccgcgcaagaactgggtcatcaccggttccgatgacatgcagatccgcgtcttcaactacaacacgttggagagagtgcacgccttcgaggctcattctgactacgtacggtgtatcgcgatacatcctacacagccatacattcttacaagcagtgatgacctgttgataaagctgtggaactgggaacgcaactgggcgtgccagcaggtgttcgagggtcacacgcattatgtgatgcagatcgtcatcaatcccaaggacaacaacacattcgcgagcgctagtctcgataccaccgtcaaagtatggcagcttggatcgtcaatctctaacttcacgctagaagggcacgagaaaggcgtgaactgcgtggattactaccacggcggagacaaaccctacctcataagtggtgctgacgatcgcctcgtcaaaatatgggactaccaaaacaaaacttgtgttcagacattagaaagtcacacacagaatgtgtcagctgtatcatttcaccccgagcttccaatactgctgaccggctcagaagatggcaccatcaggatatggcacgccggcacttacaggctcgagtcttccctaaactatggcttcgaaagagtctggactatctcttccatgcatggatctaataatgtggcaatcggttacgacgagggcacgattatgatcaaggttggccgtgaggagcccgctatttccatggacgtcaacggtggcaagattatctgggcgaagcattcggagatgcagcaagtcaatttgaaggcgctacctgaaggtaccgaaataaaagatggcgagcgtgtccccgttatggcgaaggacatgggttcctgcgagatctacccacagactattgctcacaacccgaacggccgtttcgtggttgtctgtggtgacggagaatacatcatttatactgcaatggcgctgagaaacaaggcttttggaactgcgcaagagtttgtctgggcatttgacagctcggagtatgcgacccttgaaaattccagtaccataaaggtattcaaaaacttcaaagagagaaagagcttcaagcccgagtttggtgctgaaggtatttacggcgggttcatgctgggtgtgaagtctatcagcggtgtggcattctcgttctacgactgggagaatctagaacttatcagaaggattgagatccagccccgtcacgtttactggtcggagagcgggaacttggtgtgcctggccactgacgactcgtactatgtgctcaagtacaacgccgctgtggtcactcgcgctcgggagacgaacaccaacatcacagaagatggtattgaggatgcttttgaggttgtgggcgcagtgaacgagatagtgaggactgggctatgggtgggcgactgcttcatctacacgaactcagtgaacagaataaactactacgtcggcggcgagatcgtcactatatcccacctggaccacaccatgtatatcctaggatacgtagccaaggagaacagactgtatctaaacgacaaggagttgaacatagtgtcatactccctcctgctgtcggtacttgagtatcagacggcggtgatgcgcgcagacttcgagacggccgaccgcgtgctgcccaccatacctcacgagcaccgcaccagggtcgctcacttcctagagaaacaaggcttcaagcagcaagcactagcggtatccacggagcccgaacaccagttcgaactggctctctcgctgggagaactacggagggcgaagcagcttgctgaagaagcggccgttgccgaaggagccccgtctcggtcgtcctcggcacgatggtcgcgactgggagctgcggccgccgctgccgctgacaccgagcttacgaaggcctgctaccagagcgctaaggattacagtgccttgttgctgtttgcggctagcactggtgacaagacgttgctaaggaacgtagctcagatgtcttcggaggagggggcggagaacatctcgttcgtggcgtacttcatgctgaatgatctcgacgcctgtctcaatctgctgatacagcgcaacaaactacctgaagccgccttctttgccagatcatacattccatcgaaaatgtcggaggtggtgaaactatggcgcgagtcgacatcggccaccaacaagaagctcggccagtcgctcgccgaccccgagcagtacgaaaacttgttcccggagtacctgcaatcgttggatctggagagatttcagcgcgaatacgggtatgagcagagttgcctgctagcaaacttgcccgaagattttaagacttgcaatatcgaccgcgatctcgctgccgagaaggctgaggcagagaagaacggctttaaactgccgcccattgtgcaaacttcgcaatccaccagcgcagctgttgcagacattagtttaactacatcagattgtcctacgagcgacggctccagcagtaaagctccggagcaagtaccacaggcgttgccttccgcgcctaagaaacggaacgattctctcgacatcatggacgaactagagcgcgagattgatgacatcatactggatagtacggccgacagcgtggatctgtcggatgacgcagatttgatggactaatttttaagtgtattattattattgtaattgattattttatataacagtacagatcataaaattattgtacttcataaagtgctaattccttataagattaaagtattatacagattt

SEQ ID NO: 29 - DNA, 300 bp, synthetic construct
  source 1..300: mol_type = other DNA
ggatgcttttgaggttgtgggcgcagtgaacgagatagtgaggactgggctatgggtgggcgactgcttcatctacacgaactcagtgaacagaataaactactacgtcggcggcgagatcgtcactatatcccacctggaccacaccatgtatatcctaggatacgtagccaaggagaacagactgtatctaaacgacaaggagttgaacatagtgtcatactccctcctgctgtcggtacttgagtatcagacggcggtgatgcgcgcagacttcgagacggccgaccgcgtgctgcc

SEQ ID NO: 30 - DNA, 300 bp, synthetic construct
  source 1..300: mol_type = other DNA
ggatgtttttgaggttgtgggtgtagtgaatgagatagtgaggattgggttatgggtgggtgattgttttatttatatgaatttagtgaatagaataaattattatgttggtggtgagattgttattatattttatttggattatattatgtatattttaggatatgtagttaaggagaatagattgtatttaaatgataaggagttgaatatagtgttatatttttttttgttgttggtatttgagtattagatggtggtgatgtgtgtagattttgagatggttgattgtgtgttgtt

SEQ ID NO: 31 - DNA, 794 bp, synthetic construct
  source 1..794: mol_type = other DNA
aactcgagtaatacgactcactatagggggcagcacgcggtcggccgtctcgaagtctgcgcgcatcaccgccgtctgatactcaagtaccgacagcaggagggagtatgacactatgttcaactccttgtcgtttagatacagtctgttctccttggctacgtatcctaggatatacatggtgtggtccaggtgggatatagtgacgatctcgccgccgacgtagtagtttattctgttcactgagttcgtgtagatgaagcagtcgcccacccatagcccagtcctcactatctcgttcactgcgcccacaacctcaaaagcatccgcaagctgcttcgccctccgtagttctcccagcgagagagccagttcgaactggtgttcgggctccgtggataccgctagtgcttgctgcttgaagccttgtttctctaggaagtgagcgaccctggtgcggtgctcgtgaggtatggtgggatgcttttgaggttgtgggcgcagtgaacgagatagtgaggactgggctatgggtgggcgactgcttcatctacacgaactcagtgaacagaataaactactacgtcggcggcgagatcgtcactatatcccacctggaccacaccatgtatatcctaggatacgtagccaaggagaacagactgtatctaaacgacaaggagttgaacatagtgtcatactccctcctgctgtcggtacttgagtatcagacggcggtgatgcgcgcagacttcgagacggccgaccgcgtgctgcccccgggcgtctagaga

SEQ ID NO: 32 - DNA, 794 bp, synthetic construct
  source 1..794: mol_type = other DNA
aactcgagtaatacgactcactatagggggcagcacgcggtcggccgtctcgaagtctgcgcgcatcaccgccgtctgatactcaagtaccgacagcaggagggagtatgacactatgttcaactccttgtcgtttagatacagtctgttctccttggctacgtatcctaggatatacatggtgtggtccaggtgggatatagtgacgatctcgccgccgacgtagtagtttattctgttcactgagttcgtgtagatgaagcagtcgcccacccatagcccagtcctcactatctcgttcactgcgcccacaacctcaaaagcatccgcaagctgcttcgccctccgtagttctcccagcgagagagccagttcgaactggtgttcgggctccgtggataccgctagtgcttgctgcttgaagccttgtttctctaggaagtgagcgaccctggtgcggtgctcgtgaggtatggtgggatgtttttgaggttgtgggtgtagtgaatgagatagtgaggattgggttatgggtgggtgattgttttatttatatgaatttagtgaatagaataaattattatgttggtggtgagattgttattatattttatttggattatattatgtatattttaggatatgtagttaaggagaatagattgtatttaaatgataaggagttgaatatagtgttatatttttttttgttgttggtatttgagtattagatggtggtgatgtgtgtagattttgagatggttgattgtgtgttgttcccgggcgtctagaga

SEQ ID NO: 33 - protein, 556 aa, Helicoverpa armigera
MNNNQFHDLFGSQWPPDQHGGHSSASTMLHQSQGLPQGMQLKREPHTDVQPVMHNQMGMDITSGSVADSTSPPPGSSDGMFGSSISGMFMDKKAANSIRAQIEIIPCKVCGDKSSGVHYGVITCEGCKGFFRRSQSTVVNYQCPRNKACVVDRVNRNRCQYCRLQKCLKLGMSRDAVKFGRMSKKQREKVEDEVRFHRAQMRAQTDTAPDSVYDAQQQTPSSSDQFHGHYNGYPGYGSPLSSYGYNNAGPALQSNMGGIQPQAPQQQPYDVSADYVDSTTAYEPKQTGGFLDPDFISHAEGDISKVLVKSLAEAHANTNPKLEFIHEMFRKPQDVSKLLYYSSMTYEEMWLDCADKLTGMIQNIIEFAKLIPGFMKLTQDDQILLLKSGSFELAIVRLSRLIDVNRDQVLYGDAVLSIRECVHARDPRDVALVVGIFDAAKTIARLKLTETELALYQSLVLLWPERHGVRGNPEIQCLFNMSMAAMRHEIETNHAPLKGDVTVLDTLLAKIPTFRELSLMHLEALCRFKAAHPHHVFPALYKELFSLDSVLDYTHG

SEQ ID NO: 34 - DNA, 1671 bp, synthetic construct
  source 1..1671: mol_type = other DNA
atgaacaacaaccagttccacgatttgtttgggtcccagtggcctccggaccagcacggaggtcactcgtcggcatcgacgatgctgcaccagtcccagggcctgccccagggtatgcagctgaagagagaacctcatacggatgtgcagccggtgatgcacaaccagatgggcatggacatcacgtcagggtccgtggcggacagtacatcaccgccgcctggcagcagtgatgggatgttcggctcgtctatatctggcatgtttatggataagaaagccgctaattctataagagctcaaatcgaaataataccatgcaaggtgtgtggggacaagtcatctggagtccactatggcgtcatcacctgcgaggggtgcaagggtttcttcaggcgatcacagagtacagtggtgaactaccagtgtccacggaataaagcctgcgtggtcgaccgggtcaacagaaatcgttgtcagtattgcagattgcagaaatgcctcaaacttggaatgagccgtgatgcggtgaagtttggacggatgtcgaaaaagcaacgtgagaaggtggaagatgaggtgagattccatcgcgcacagatgagagctcagactgacacggcgcccgactcagtatacgacgctcaacagcagacgccgagctcaagcgaccagttccatggtcactacaatggctacccgggctacgggtcgccgctgtcctcatatgggtacaacaacgctggaccggcgctacagtccaacatgggaggcatacagcctcaagcacctcaacagcagccctatgacgtgtcggctgattacgtggactccaccacggcttacgagccgaaacagaccgggggattcttggatcctgattttattagtcatgcggagggtgacatcagcaaagttctggtgaagagtttggcagaagcacacgcaaataccaaccccaaactggagttcattcatgagatgttcagaaagccacaggatgtctccaagcttctatactatagttcaatgacgtacgaggaaatgtggctggattgtgcagacaaattaactgggatgattcagaatattatcgagttcgctaaacttatacccgggttcatgaaactgactcaggatgatcagattttgctcttgaaatctggctcgttcgagctagccatagtccggctgtcgcgactgatagacgtcaatcgtgaccaagtgctatacggtgatgcggtgctgtccatcagagaatgtgtgcatgcacgtgacccccgcgacgtggcgctagtggttggtatcttcgacgcagcgaagacgatcgctcgactcaaactcactgagactgaactggcgctataccagagccttgtgctattgtggccagagcggcacggcgtccgcggcaaccctgagatccagtgcctgttcaacatgtcgatggcggcgatgcggcacgagatcgagaccaaccacgcccccctcaagggagacgtcactgtactggacacgctgctcgccaagatacccaccttcagagaactatcgctgatgcacctggaagcgttgtgccgcttcaaggccgcccacccacaccacgtgttcccggccctctacaaggagctgttctcactcgacagcgtcctggactacacccacggttaa

SEQ ID NO: 35 - DNA, 300 bp, synthetic construct
  source 1..300: mol_type = other DNA
ggaccagcacggaggtcactcgtcggcatcgacgatgctgcaccagtcccagggcctgccccagggtatgcagctgaagagagaacctcatacggatgtgcagccggtgatgcacaaccagatgggcatggacatcacgtcagggtccgtggcggacagtacatcaccgccgcctggcagcagtgatgggatgttcggctcgtctatatctggcatgtttatggataagaaagccgctaattctataagagctcaaatcgaaataataccatgcaaggtgtgtggggacaagtcatctgg

SEQ ID NO: 36 - DNA, 300 bp, synthetic construct
  source 1..300: mol_type = other DNA
ggattagtatggaggttatttgttggtattgatgatgttgtattagttttagggtttgttttagggtatgtagttgaagagagaattttatatggatgtgtagttggtgatgtataattagatgggtatggatattatgttagggtttgtggtggatagtatattattgttgtttggtagtagtgatgggatgtttggtttgtttatatttggtatgtttatggataagaaagttgttaattttataagagtttaaattgaaataatattatgtaaggtgtgtggggataagttatttgg

SEQ ID NO: 37 - DNA, 794 bp, synthetic construct
  source 1..794: mol_type = other DNA
aactcgagtaatacgactcactatagggccagatgacttgtccccacacaccttgcatggtattatttcgatttgagctcttatagaattagcggctttcttatccataaacatgccagatatagacgagccgaacatcccatcactgctgccaggcggcggtgatgtactgtccgccacggaccctgacgtgatgtccatgcccatctggttgtgcatcaccggctgcacatccgtatgaggttctctcttcagctgcataccctggggcaggccctgggactggtgcagcatcgtcgatgccgacgagtgacctccgtgctggtccttctgcaatctgcaatactgacaacgatttctgttgacccggtcgaccacgcaggctttattccgtggacactggtagttcaccactgtactctgtgatcgcctgaagaaacccttgcacccctcgcaggtgatgacgccatagtggactggaccagcacggaggtcactcgtcggcatcgacgatgctgcaccagtcccagggcctgccccagggtatgcagctgaagagagaacctcatacggatgtgcagccggtgatgcacaaccagatgggcatggacatcacgtcagggtccgtggcggacagtacatcaccgccgcctggcagcagtgatgggatgttcggctcgtctatatctggcatgtttatggataagaaagccgctaattctataagagctcaaatcgaaataataccatgcaaggtgtgtggggacaagtcatctggcccgggcgtctagaga

SEQ ID NO: 38 - DNA, 794 bp, synthetic construct
  source 1..794: mol_type = other DNA
aactcgagtaatacgactcactatagggccagatgacttgtccccacacaccttgcatggtattatttcgatttgagctcttatagaattagcggctttcttatccataaacatgccagatatagacgagccgaacatcccatcactgctgccaggcggcggtgatgtactgtccgccacggaccctgacgtgatgtccatgcccatctggttgtgcatcaccggctgcacatccgtatgaggttctctcttcagctgcataccctggggcaggccctgggactggtgcagcatcgtcgatgccgacgagtgacctccgtgctggtccttctgcaatctgcaatactgacaacgatttctgttgacccggtcgaccacgcaggctttattccgtggacactggtagttcaccactgtactctgtgatcgcctgaagaaacccttgcacccctcgcaggtgatgacgccatagtggactggattagtatggaggttatttgttggtattgatgatgttgtattagttttagggtttgttttagggtatgtagttgaagagagaattttatatggatgtgtagttggtgatgtataattagatgggtatggatattatgttagggtttgtggtggatagtatattattgttgtttggtagtagtgatgggatgtttggtttgtttatatttggtatgtttatggataagaaagttgttaattttataagagtttaaattgaaataatattatgtaaggtgtgtggggataagttatttggcccgggcgtctagaga

SEQ ID NO: 39 - protein, 620 aa, Helicoverpa armigera
MARALTGGGLRTIADEEIEERFGFVFAVSGPVVTAEKMSGSAMYELVRVGYNELVGEIIRLEGDMATIQVYEETSGVTVGDPVLRTGKPLSVELGPGILGSIFDGIQRPLKDINELTQSIYIPKGVNVPSLARDVSWEFVPMNVKTGSHITGGDLYGLVHENTLVKHRMLIPPKAKGTVTYIAPAGNYKVTDVVLETEFDGEKEKYTMLQVWPVRQPRPVTEKLPANHPLLTGQRVLDSLFPCVQGGTTAIPGAFGCGKTVISQALSKYSNSDVIVYVGCGERGNEMSEVLRDFPELTVEIEGVTESIMKRTALVANTSNMPVAAREASIYTGITLSEYFRDMGYNVSMMADSTSRWAEALREISGRLAEMPADSGYPAYLGARLASFYERAGRVKCLGNPDREGSVSIVGAVSPPGGDFSDPVTAATLGIVQVFWGLDKKLAQRKHFPSINWLISYSKYMRALDDFYDKNYVEFVPLRTKVKEILQEEEDLSEIVQLVGKASLAETDKITLEVAKLLKDDFLQQNSYSAYDRFCPFYKTVGMLKNIIAFYDMSRHAVESTAQSDNKVTWNVIRDAMGNVLYQLSSMKFKDPVKDGEAKIKADFDQLLEDMAAAFRNLED

SEQ ID NO: 40 - DNA, 2645 bp, synthetic construct
  source 1..2645: mol_type = other DNA
gtgtgtggaatagacaatgaggcatagacttaggtcagctgacttcatttctatcactggtcttgtgataagttgtgatcctccgccattctcggtccgtcttctccatctttctcgtcccgaagaattaataataattcaccatggctagagcgctcaccgggggtggcttgaggacgatcgccgatgaagagatcgaggagaggttcggcttcgtatttgccgtatccggtcccgtcgtcacggcggagaagatgtccggatcggctatgtacgagctggtgcgtgtcggatacaacgagctggtcggagagatcatccgtctggagggtgacatggccaccatccaggtatacgaggaaacttcaggtgtaaccgttggtgaccccgtactccgtaccggcaagcccctgtccgtggagttgggccccggtatcctgggctccatctttgacggtatccagcgtcccctgaaggacatcaatgagctcacacagtccatctacatccccaagggtgtgaacgtaccctctctggctagggatgtcagctgggaattcgttcccatgaatgttaagacgggctcccacatcactggcggagacctgtacggtctggtgcacgagaacacgctggtgaagcaccgcatgctgatcccgcccaaggccaagggtaccgtcacatatatcgcgcccgctggcaactacaaagtcactgacgtagtgctggagacggagttcgacggcgagaaggagaagtacacgatgttgcaggtgtggccggtgcgtcagccgcgacccgtcaccgagaagctccccgccaaccatccgctgctcaccggccagagggtgctcgactcactcttcccttgcgtccagggaggtacgacagccatccccggagctttcggttgcggcaagactgtcatctcgcaggcgctgtccaagtactccaactcggatgtcattgtgtacgtcgggtgcggagagcgtggtaacgagatgtccgaagtactgcgtgacttccccgagctgacggtggagatcgagggcgtgacggagtccatcatgaagcgaactgccctcgtcgccaacacctccaacatgcctgtcgccgcccgagaggcttccatctacactggtatcactctatccgagtacttccgtgacatgggttacaacgtgtccatgatggctgactccacgtctcgttgggccgaggctctgcgtgagatctcgggtcgtctggcggagatgccggccgactccggctaccccgcctacctgggtgctaggttggcttccttctacgagagagccggacgtgtcaagtgtctgggtaaccccgacagggaaggttccgtatccatcgtgggtgccgtatctccccctggtggtgacttctctgaccctgtaactgccgccaccctgggtatcgtgcaggtgttctggggcttggacaagaagctggcgcagcgcaagcacttcccgtccatcaactggctcatctcctacagcaagtacatgcgagccctcgacgacttctacgacaagaactacgtcgagttcgtgcctcttaggactaaggtcaaggagatcctgcaggaagaagaggatctgtcagaaatcgtgcagctggtcggtaaggcatcgctggccgagaccgacaagatcaccctcgaggtcgccaagctgctcaaagacgacttcttgcagcagaacagctactctgcatacgatcgtttctgtccgttctacaagacggtgggcatgctgaagaacatcatcgcgttctacgacatgtcgcgccacgccgtggagtccaccgcgcagtccgacaacaaggtcacgtggaacgtcatccgcgacgccatgggcaacgtgctctaccagctctcctccatgaagttcaaggaccccgtgaaagatggcgaggctaagatcaaggcggacttcgaccagctgctcgaggacatggcggccgccttccgcaacctcgaagactagacccgcacactgtaacataatgtcgtacaatgtactacgcagacgcgtattgttaatacactaatagatgtcttgtctacgtagtgtgaataggtttaattatattattactcgtatttcaatgcagaattcgtgattccaattgttgaaatcggtcgtcggcggccgttggcggatccccacaactctccatttgtaaatatacgaacaaatgccgccggcccgccgcgactacagttaattagttgttatttatataaaatatatactatttatacactaacaatgaattattttatgcccgtttgtatttcgcgtgttataaaagtcataatatcgagtaccctcttgcattccacgttattattagagttttaatcgaagtaacatgtatttgttgaaaaataaaagctacagttttttacgcttttcgtgtctcgatatcagtgttaccagcattttgatgtatcggaaatgtataagagttcagtattatgtttactttgatagttctcgctgtagctagcggcgtctgtacaaacaatcatgcgatataataacgtatattacttgctgtttgaacctctgtgacgtttgttgaggagacgaaagttataaataaataaatattaaagtgta

SEQ ID NO: 41 - DNA, 300 bp, synthetic construct
  source 1..300: mol_type = other DNA
gggtggcttgaggacgatcgccgatgaagagatcgaggagaggttcggcttcgtatttgccgtatccggtcccgtcgtcacggcggagaagatgtccggatcggctatgtacgagctggtgcgtgtcggatacaacgagctggtcggagagatcatccgtctggagggtgacatggccaccatccaggtatacgaggaaacttcaggtgtaaccgttggtgaccccgtactccgtaccggcaagcccctgtccgtggagttgggccccggtatcctgggctccatctttgacggtatcca

SEQ ID NO: 42 - DNA, 300 bp, synthetic construct
  source 1..300: mol_type = other DNA
gggtggtttgaggatgattgttgatgaagagattgaggagaggtttggttttgtatttgttgtatttggttttgttgttatggtggagaagatgtttggattggttatgtatgagttggtgtgtgttggatataatgagttggttggagagattatttgtttggagggtgatatggttattatttaggtatatgaggaaattttaggtgtaattgttggtgattttgtattttgtattggtaagtttttgtttgtggagttgggttttggtattttgggttttatttttgatggtattta

SEQ ID NO: 43 - DNA, 794 bp, synthetic construct
  source 1..794: mol_type = other DNA
aactcgagtaatacgactcactatagggtggataccgtcaaagatggagcccaggataccggggcccaactccacggacaggggcttgccggtacggagtacggggtcaccaacggttacacctgaagtttcctcgtatacctggatggtggccatgtcaccctccagacggatgatctctccgaccagctcgttgtatccgacacgcaccagctcgtacatagccgatccggacatcttctccgccgtgacgacgggaccggatacggcaaatacgaagccgaacctctcctcgatctcttcatcggcgatcgtcctcaagccacccccgtacaggtctccgccagtgatgtgggagcccgtcttaacattcatgggaacgaattcccagctgacatccctagccagagagggtacgttcacacccttggggatgtagatggactgtgtgagctcattgatgtccttcaggggacgcgggtggcttgaggacgatcgccgatgaagagatcgaggagaggttcggcttcgtatttgccgtatccggtcccgtcgtcacggcggagaagatgtccggatcggctatgtacgagctggtgcgtgtcggatacaacgagctggtcggagagatcatccgtctggagggtgacatggccaccatccaggtatacgaggaaacttcaggtgtaaccgttggtgaccccgtactccgtaccggcaagcccctgtccgtggagttgggccccggtatcctgggctccatctttgacggtatccacccgggcgtctagaga

SEQ ID NO: 44 - DNA, 794 bp, synthetic construct
  source 1..794: mol_type = other DNA
aactcgagtaatacgactcactatagggtggataccgtcaaagatggagcccaggataccggggcccaactccacggacaggggcttgccggtacggagtacggggtcaccaacggttacacctgaagtttcctcgtatacctggatggtggccatgtcaccctccagacggatgatctctccgaccagctcgttgtatccgacacgcaccagctcgtacatagccgatccggacatcttctccgccgtgacgacgggaccggatacggcaaatacgaagccgaacctctcctcgatctcttcatcggcgatcgtcctcaagccacccccgtacaggtctccgccagtgatgtgggagcccgtcttaacattcatgggaacgaattcccagctgacatccctagccagagagggtacgttcacacccttggggatgtagatggactgtgtgagctcattgatgtccttcaggggacgcgggtggtttgaggatgattgttgatgaagagattgaggagaggtttggttttgtatttgttgtatttggttttgttgttatggtggagaagatgtttggattggttatgtatgagttggtgtgtgttggatataatgagttggttggagagattatttgtttggagggtgatatggttattatttaggtatatgaggaaattttaggtgtaattgttggtgattttgtattttgtattggtaagtttttgtttgtggagttgggttttggtattttgggttttatttttgatggtatttacccgggcgtctagaga

SEQ ID NO: 45 - protein, 299 aa, Helicoverpa armigera
MGSFLILIAIALSVIVAVSSNAVDSNRIVGGFVSPEGVFKYMISLQELSKIQDYRRGHRCGGALISYQHALTAASCTFNYNNGALTPINVAEFRVFAGSVLLTNDASEDHVRSISNIRVHPSYLTSQPFADDTAVITLTVPFPNNVVKPVPLSEREVSPEEFGVCELSGWGSRNSSATESAELIYSIHTIFDWLACAIAYGALSPQVVIFPGIICAYADKTIAVGCTGDIGNPLVCHDSLTGIAVKTQDCSMRAFPEVYTKVSAYIPWINRVMNGASSIMQPGIALLTTFVVIASKIIT

SEQ ID NO: 46 - DNA, 1025 bp, synthetic construct
  source 1..1025: mol_type = other DNA
atctagttaagttgttgattgtggtcaagaaatggggtcttttctaattttaattgcaattgcactcagtgtgattgtggcagtctcttcaaatgccgttgattcaaaccgaatcgtaggtggatttgtttcaccagagggcgtgttcaaatatatgatttcactgcaagaattaagcaaaatacaagattatagacgaggacacagatgtggaggggctctgatctcataccagcatgcactgacagctgcatcgtgcaccttcaattataacaatggtgcgctcacacctattaacgtggctgaattccgtgtgttcgctggttcagtgttgcttactaacgacgcatctgaagatcacgttcggagtatctcaaacatcagagttcacccctcatacttgacaagccaaccgtttgctgatgataccgctgttataaccctcaccgttccattccccaacaacgtggtgaaaccagtccctttatctgaaagagaagttagtcctgaagaattcggagtctgcgaactatcgggttggggaagtcgaaactcatcagccactgaaagtgcggaattaatatactcaatccatacaatttttgattggcttgcttgtgcgattgcgtacggtgctctttctccgcaagttgttatattccccggtataatctgcgcgtacgccgataaaacaatagcagttgggtgcactggcgatattggcaaccccttggtctgccatgatagccttacgggcattgctgtcaagacccaagactgttctatgagagcttttcctgaggtctacactaaggtgtccgcgtacatcccatggattaaccgagtcatgaacggtgcatcctcaatcatgcaacctggaatagctttgttaactacgttcgtagtcatagcttctaaaatcataacctagaagcttctaaaagtagctaaggaggaataattatttttttatgttaatatcccactataatttatttcatcttcaataaatgttatttgaaagc

SEQ ID NO: 47 - DNA, 300 bp, synthetic construct
  source 1..300: mol_type = other DNA
cgattgcgtacggtgctctttctccgcaagttgttatattccccggtataatctgcgcgtacgccgataaaacaatagcagttgggtgcactggcgatattggcaaccccttggtctgccatgatagccttacgggcattgctgtcaagacccaagactgttctatgagagcttttcctgaggtctacactaaggtgtccgcgtacatcccatggattaaccgagtcatgaacggtgcatcctcaatcatgcaacctggaatagctttgttaactacgttcgtagtcatagcttctaaaa

SEQ ID NO: 48 - DNA, 300 bp, synthetic construct
  source 1..300: mol_type = other DNA
tgattgtgtatggtgttttttttttgtaagttgttatattttttggtataatttgtgtgtatgttgataaaataatagtagttgggtgtattggtgatattggtaattttttggtttgttatgatagttttatgggtattgttgttaagatttaagattgttttatgagagttttttttgaggtttatattaaggtgtttgtgtatattttatggattaattgagttatgaatggtgtatttttaattatgtaatttggaatagttttgttaattatgtttgtagttatagtttttaaaa

SEQ ID NO: 49 - DNA, 794 bp, synthetic construct
  source 1..794: mol_type = other DNA
aactcgagtaatacgactcactatagggttttagaagctatgactacgaacgtagttaacaaagctattccaggttgcatgattgaggatgcaccgttcatgactcggttaatccatgggatgtacgcggacaccttagtgtagacctcaggaaaagctctcatagaacagtcttgggtcttgacagcaatgcccgtaaggctatcatggcagaccaaggggttgccaatatcgccagtgcacccaactgctattgttttatcggcgtacgcgcagattataccggggaatataacaacttgcggagaaagagcaccgtacgcaatcgcacaagcaagccaatcaaaaattgtatggattgagtatattaattccgcactttcagtggctgatgagtttcgacttccccaacccgatagttcgcagactccgaattcttcaggactaacttctctttcagataaagggactggtttcacgattgcgtacggtgctctttctccgcaagttgttatattccccggtataatctgcgcgtacgccgataaaacaatagcagttgggtgcactggcgatattggcaaccccttggtctgccatgatagccttacgggcattgctgtcaagacccaagactgttctatgagagcttttcctgaggtctacactaaggtgtccgcgtacatcccatggattaaccgagtcatgaacggtgcatcctcaatcatgcaacctggaatagctttgttaactacgttcgtagtcatagcttctaaaacccgggcgtctagaga

SEQ ID NO: 50 - DNA, 794 bp, synthetic construct
  source 1..794: mol_type = other DNA
aactcgagtaatacgactcactatagggttttagaagctatgactacgaacgtagttaacaaagctattccaggttgcatgattgaggatgcaccgttcatgactcggttaatccatgggatgtacgcggacaccttagtgtagacctcaggaaaagctctcatagaacagtcttgggtcttgacagcaatgcccgtaaggctatcatggcagaccaaggggttgccaatatcgccagtgcacccaactgctattgttttatcggcgtacgcgcagattataccggggaatataacaacttgcggagaaagagcaccgtacgcaatcgcacaagcaagccaatcaaaaattgtatggattgagtatattaattccgcactttcagtggctgatgagtttcgacttccccaacccgatagttcgcagactccgaattcttcaggactaacttctctttcagataaagggactggtttcatgattgtgtatggtgttttttttttgtaagttgttatattttttggtataatttgtgtgtatgttgataaaataatagtagttgggtgtattggtgatattggtaattttttggtttgttatgatagttttatgggtattgttgttaagatttaagattgttttatgagagttttttttgaggtttatattaaggtgtttgtgtatattttatggattaattgagttatgaatggtgtatttttaattatgtaatttggaatagttttgttaattatgtttgtagttatagtttttaaaacccgggcgtctagaga

SEQ ID NO: 51 - protein, 557 aa, Helicoverpa armigera
MVETDPGPKKSHNCDVKRDLQLNGPGSKTAELGVVPSLKTAVKLDPEKGSNSEKADFERAIELTGYGRFHYLLLAVCGLVSTSEEMDVISMSFILPSAQCDLDLTTQTKGWLNSIIFIGMMVGAYAWGSVADSLGRKRVLIAISVVNALAIVASSFSQNYELFMLFRFINGAALGGSGPVIWSYFAEFQPKNKRGAMLSFMAAFWTLGNLFVAGLAWVIIPSEIGGVTNGFVYNSWRIFLLVMSLPSFLVAALLFLLPESPKFLISTGRHDEALEVFKGIYMMNTGRSKEEYPVKQLLIEECYHTKPEKQIEVKEPKSKIRKMFSDIVEHSKQLFVPPILKFTTISITINFTFHIGYYGLMMWFPEMFNRFDEWSRTHDNAEADICMVTNYVTQYGTHSTAAQCDSHIHSDVFMESLITVAAAIPSNIFAVLGMDRLGRKFFLVFATFSAGLCSAAMYFVYNKTNNLIVSAIFSSVISCGNASLDCLITEVFPTNLRATGVAVSMVAARLGGIIGNVVIAALLDTYCPAPTFIVAVLLASGGLMCLFLPNTTRQALS

SEQ ID NO: 52 - DNA, 2661 bp, synthetic construct
  source 1..2661: mol_type = other DNA
gagcacacgcctcgcacgctcgcccgctcgcccgccagcgctcgccactcgaccacaacaaacagtgcgcgcagcctccgccactcgcgagccccgggatcacatacccctctatgcggacgttcaacatcggaagtgtcgtgaaatgttttacgaaagtgttatttgacgcgagttttcatcacttcttgtgacattcagcatttctctcggcgacttcacgaaacaatgctcatatttggaacttttgactgataatcgtatttcattttgactgtgcatctctgttttgacgagccgtgcgccgttgtaagcaacccacaacccagtaagactagactcgtccgcgcaaccgccgacataccaagacacccgcccgcataggatggtagaaactgaccccgggcccaagaaatctcacaattgtgatgtaaaaagggacctccaactaaacggcccaggttccaaaaccgccgagcttggcgtcgtaccgtcactgaagacagcagtcaagctcgaccccgagaaaggctccaactcggagaaggctgacttcgaacgggctatcgaattaaccggatatggacggttccactacctcctgctggcggtgtgcggcctggtcagcacctccgaggagatggacgtcatctccatgtccttcatcctcccttcggcacagtgcgacctagacctcaccacgcagactaaaggatggctaaacagtataatcttcatcgggatgatggtgggagcgtacgcatgggggtcggtcgcggactcgctgggacgcaagcgcgtcctgatcgcgatctccgtcgtgaacgcgctcgccatcgtcgcctcctccttcagtcagaactacgagctcttcatgctgttccgcttcatcaatggtgccgctttaggaggatcaggtcccgtgatctggtcatacttcgcggagttccagccgaagaacaagcgaggagctatgttgagcttcatggcagctttctggacgctcggcaacctgttcgtcgctggtttggcgtgggtcatcatccctagcgaaatcggaggagtaacgaatggcttcgtatacaactcatggcgtatcttcttactggtaatgtcactaccctcgttcttggtagccgcattactgttcctgctgcccgagtctcccaagttcttgatatcaaccggtcgtcatgacgaagccttggaagtattcaaaggcatctacatgatgaacactggccgcagcaaagaggagtacccagtcaaacagttgctgatcgaagagtgctaccacactaagccggagaaacaaatcgaagttaaggaaccaaaatctaagataaggaaaatgttcagcgacattgtggaacacagcaaacagctatttgtgccgcctatactcaagtttactacgatctctatcacaatcaacttcacattccatattgggtactacggcctcatgatgtggttccccgagatgttcaaccgtttcgacgagtggtcccgtacgcacgacaacgcggaagccgacatttgcatggtgacgaattacgtcacgcagtacggcacgcactcgacagcggcgcagtgcgactcccatatacactccgatgtgttcatggagtcgctcatcactgtggctgctgccataccatctaatatatttgcggtactcggcatggacagactcgggaggaagtttttccttgtgttcgcaacattctccgctggcttatgttcagcagctatgtacttcgtgtacaataagactaacaacttaatcgtaagcgccatcttcagttcggtgatatcctgcggtaacgcctcactcgactgcctcatcacagaagtcttccctaccaacttacgtgctactggtgtagcggtatcgatggtggcggctcgactgggcggcatcatcggcaacgtggtgatcgcggcgttgctggacacctactgccccgcgcccaccttcatcgtcgccgtgctgctcgccagcggcggtctcatgtgcctcttcctgcccaacaccacgcggcaggccctctcctaacaccgccatctttttcttgccgcataaacgtcaatttcaaagactaacggcattttataagattttttttggcctgccatgtgatatataaaacattattttaagattttaagcgttaaatctcatcgaaagatccattgatgtaactagttttttttacatatttgtacaatagtttttatattaatttaaatgaaaatgttttatttatgaactaaatgaaagtacgaacgatacatattcctagtaaataaagtaattgactgttgtaggttgttataacttattacgtttatacatttgtttgaaattgtaaaccagaataaagctttgcgtcagatcttttaatggataaagtattataggataagtatcatttcattaccatgttgtatattattgcaactattttaatattacgaatttataatatcattgttatcgtagtttgtagttgagtagtacgtagtaaaattataaaaacacgagttgtgatataataaagtttgtatcaaatctttgcattgtatataagtgcatttttcaaaaccatggaacaccttattaattacaatgcaatatattttcctaaattcatttca

SEQ ID NO: 53 - DNA, 300 bp, synthetic construct
  source 1..300: mol_type = other DNA
aacctgttcgtcgctggtttggcgtgggtcatcatccctagcgaaatcggaggagtaacgaatggcttcgtatacaactcatggcgtatcttcttactggtaatgtcactaccctcgttcttggtagccgcattactgttcctgctgcccgagtctcccaagttcttgatatcaaccggtcgtcatgacgaagccttggaagtattcaaaggcatctacatgatgaacactggccgcagcaaagaggagtacccagtcaaacagttgctgatcgaagagtgctaccacactaagccggag

SEQ ID NO: 54 - DNA, 300 bp, synthetic construct
  source 1..300: mol_type = other DNA
aatttgtttgttgttggtttggtgtgggttattatttttagtgaaattggaggagtaatgaatggttttgtatataatttatggtgtatttttttattggtaatgttattatttttgtttttggtagttgtattattgtttttgttgtttgagttttttaagtttttgatattaattggttgttatgatgaagttttggaagtatttaaaggtatttatatgatgaatattggttgtagtaaagaggagtatttagttaaatagttgttgattgaagagtgttattatattaagttggag

SEQ ID NO: 55 - DNA, 794 bp, synthetic construct
  source 1..794: mol_type = other DNA
aactcgagtaatacgactcactatagggctccggcttagtgtggtagcactcttcgatcagcaactgtttgactgggtactcctctttgctgcggccagtgttcatcatgtagatgcctttgaatacttccaaggcttcgtcatgacgaccggttgatatcaagaacttgggagactcgggcagcaggaacagtaatgcggctaccaagaacgagggtagtgacattaccagtaagaagatacgccatgagttgtatacgaagccattcgttactcctccgatttcgctagggatgatgacccacgccaaaccagcgacgaacaggttgtagtacccaatatggaatgtgaagttgattgtgatagagatcgtagtaaacttgagtataggcggcacaaatagctgtttgctgtgttccacaatgtcgctgaacattttccttatcttagattttggttccttaacttcgatttgtttaacctgttcgtcgctggtttggcgtgggtcatcatccctagcgaaatcggaggagtaacgaatggcttcgtatacaactcatggcgtatcttcttactggtaatgtcactaccctcgttcttggtagccgcattactgttcctgctgcccgagtctcccaagttcttgatatcaaccggtcgtcatgacgaagccttggaagtattcaaaggcatctacatgatgaacactggccgcagcaaagaggagtacccagtcaaacagttgctgatcgaagagtgctaccacactaagccggagcccgggcgtctagaga

SEQ ID NO: 56 - DNA, 794 bp, synthetic construct
  source 1..794: mol_type = other DNA
aactcgagtaatacgactcactatagggctccggcttagtgtggtagcactcttcgatcagcaactgtttgactgggtactcctctttgctgcggccagtgttcatcatgtagatgcctttgaatacttccaaggcttcgtcatgacgaccggttgatatcaagaacttgggagactcgggcagcaggaacagtaatgcggctaccaagaacgagggtagtgacattaccagtaagaagatacgccatgagttgtatacgaagccattcgttactcctccgatttcgctagggatgatgacccacgccaaaccagcgacgaacaggttgtagtacccaatatggaatgtgaagttgattgtgatagagatcgtagtaaacttgagtataggcggcacaaatagctgtttgctgtgttccacaatgtcgctgaacattttccttatcttagattttggttccttaacttcgatttgtttaatttgtttgttgttggtttggtgtgggttattatttttagtgaaattggaggagtaatgaatggttttgtatataatttatggtgtatttttttattggtaatgttattatttttgtttttggtagttgtattattgtttttgttgtttgagttttttaagtttttgatattaattggttgttatgatgaagttttggaagtatttaaaggtatttatatgatgaatattggttgtagtaaagaggagtatttagttaaatagttgttgattgaagagtgttattatattaagttggagcccgggcgtctagaga

SEQ ID NO: 57 - protein, 151 aa, Helicoverpa armigera
MDTDDDQKMAMLRKAFQMFDTTKSGYIDVLKISTILNTMGQLFDDSELQALIDENDPENSGKINFDGFCNIASHFLEEEDAEAMQQELKEAFRLYDREGNGYITTSTLKEILAALDDKLSSSDLDGIIAEIDTDGSGTVDFDEFMEMMTGD

SEQ ID NO: 58 - DNA, 955 bp, synthetic construct
  source 1..955: mol_type = other DNA
tatttttgtattccagtctgaggaggaaggaatgctataaataagggtgccacatcgacgtaaaatctagtgtaacaacactcgcactcggattcggttcttgttactactgccaaccctacacacacaccatggacactgacgatgaccagaagatggccatgctgcgcaaggccttccagatgttcgataccaccaagtcgggatacattgatgtcctgaagatctccaccatcctcaacactatgggccagctcttcgatgactctgaactgcaggctctcatcgatgaaaatgacccagaaaactctggcaaaatcaacttcgatggcttctgcaacatcgcttcccacttcttggaggaggaagacgctgaggctatgcaacaggaactgaaggaagctttcaggttgtacgaccgtgaaggtaacggttacatcaccacttccaccctcaaggaaatcctggccgcccttgacgacaagctcagctcaagcgacttggacggcatcatcgccgaaattgacactgacggatccggaaccgtcgatttcgatgaattcatggagatgatgactggagactaagcaaatcgtctattactgttactattatagtattattatattttagatgctatttttattgtatttaaaaagttaagactttgacactagtacaaaaccaaatgcggtattagcatggttgtttaattaaattgcacattttactctatcttatagataggcacttgttaatataatttattagtctacaaaggtaggccaggaatcattttgagttcgttccaaagaattatttccggaataattcacatccaacgaattctagaacaattataatgctaatatttataacgcattgcactttattttggaaatatacgtaaaataacatcattattataacatcaatacacaaaacgacagtcttt

SEQ ID NO: 59 - DNA, 300 bp, synthetic construct
  source 1..300: mol_type = other DNA
tgacgatgaccagaagatggccatgctgcgcaaggccttccagatgttcgataccaccaagtcgggatacattgatgtcctgaagatctccaccatcctcaacactatgggccagctcttcgatgactctgaactgcaggctctcatcgatgaaaatgacccagaaaactctggcaaaatcaacttcgatggcttctgcaacatcgcttcccacttcttggaggaggaagacgctgaggctatgcaacaggaactgaaggaagctttcaggttgtacgaccgtgaaggtaacggttacat

SEQ ID NO: 60 - DNA, 300 bp, synthetic construct
  source 1..300: mol_type = other DNA
tgatgatgattagaagatggttatgttgtgtaaggttttttagatgtttgatattattaagttgggatatattgatgttttgaagatttttattatttttaatattatgggttagttttttgatgattttgaattgtaggtttttattgatgaaaatgatttagaaaattttggtaaaattaattttgatggtttttgtaatattgttttttattttttggaggaggaagatgttgaggttatgtaataggaattgaaggaagtttttaggttgtatgattgtgaaggtaatggttatat

SEQ ID NO: 61 - DNA, 792 bp, synthetic construct
  source 1..792: mol_type = other DNA
aactcgagtaatacgactcactatagggatgtaaccgttaccttcacggtcgtacaacctgaaagcttccttcagttcctgttgcatagcctcagcgtcttcctcctccaagaagtgggaagcgatgttgcagaagccatcgaagttgattttgccagagttttctgggtcattttcatcgatgagagcctgcagttcagagtcatcgaagagctggcccatagtgttgaggatggtggagatcttcaggacatcaatgtatcccgacttggtggtatcgaacatctggaaggccttgcgcagcatggccatcttctggtcatcgtcattagtctccagtcatcatctccatgaattcatcgaaatcgacggttccggatccgtcagtgtcaatttcggcgatgatgccgtccaagtcgcttgagctgagcttgtcgtcaagggcggccaggatttccttgagggtggaagtggtgtgacgatgaccagaagatggccatgctgcgcaaggccttccagatgttcgataccaccaagtcgggatacattgatgtcctgaagatctccaccatcctcaacactatgggccagctcttcgatgactctgaactgcaggctctcatcgatgaaaatgacccagaaaactctggcaaaatcaacttcgatggcttctgcaacatcgcttcccacttcttggaggaggaagacgctgaggctatgcaacaggaactgaaggaagctttcaggttgtacgaccgtgaaggtaacggttacatcccgggcgtctagaga

SEQ ID NO: 62 - DNA, 792 bp, synthetic construct
  source 1..792: mol_type = other DNA
aactcgagtaatacgactcactatagggatgtaaccgttaccttcacggtcgtacaacctgaaagcttccttcagttcctgttgcatagcctcagcgtcttcctcctccaagaagtgggaagcgatgttgcagaagccatcgaagttgattttgccagagttttctgggtcattttcatcgatgagagcctgcagttcagagtcatcgaagagctggcccatagtgttgaggatggtggagatcttcaggacatcaatgtatcccgacttggtggtatcgaacatctggaaggccttgcgcagcatggccatcttctggtcatcgtcattagtctccagtcatcatctccatgaattcatcgaaatcgacggttccggatccgtcagtgtcaatttcggcgatgatgccgtccaagtcgcttgagctgagcttgtcgtcaagggcggccaggatttccttgagggtggaagtggtgtgatgatgattagaagatggttatgttgtgtaaggttttttagatgtttgatattattaagttgggatatattgatgttttgaagatttttattatttttaatattatgggttagttttttgatgattttgaattgtaggtttttattgatgaaaatgatttagaaaattttggtaaaattaattttgatggtttttgtaatattgttttttattttttggaggaggaagatgttgaggttatgtaataggaattgaaggaagtttttaggttgtatgattgtgaaggtaatggttatatcccgggcgtctagaga

SEQ ID NO: 63 - protein, 4213 aa, Helicoverpa armigera
MSEGDVSLIRDEDLLRRMWQQTEDFSRKKEIRAHMYRLREERLRDLYSPEPPREGKGSEFSAAQGHVKSFADQSFQSMKSKEVRDAGSPPKEFTYRGQDLKALSNAGWNVESENETTDDGHTHVKSVHANIEGRYDVDGGKGQFAAVDHHKEAVTEYHDDNSSLKRNETSSNTAAREHVVRKTDDGTQISSTTSSSTSSSKFQQMSSTRHETVPYLTNDDYDLRSYDTNRNEDSSSVTRRQISKTNDYEQNLRNNDYETNLRKNDYQQNIIRNEYEQNVKKNEYEHVVRKNDYEQNLTRNDYETGELVSRKVDYPDDNTRVIVETRCLPDGTRVTSTKREFRAPVQSTRSEHSYQTRENKSYSSQQRSDAKESSKIIRHVIDDRATDIVDSQRNVDDHDFKRQIAEYTSKDNKDYSDVQRYETKVNKTNIDHSKLDDEYSKTTRHVTRVNKVIESTDKDDYVNTQFTNQTHDRQYQINDVRKETAVREIRPEPSPPRDQRPHTVTPQNDRPKSYTPRHDRPETMTPAENRRDTETPKNNRHDITSPRDNYREPVRPRDDQPVPVKDYVQHQTKDTEERVHHVTRDQKIEETVERKTSSDHYQTTYQTDFPQKKISNDWSPSHQAWASTLRADTPTTTRPSTRASSPGSRTFKSSSSSLRSSVSPDKISRKPSSRGGSPNKIDRHSPTRNDKNSTTHSTHSVTEVKTHKYTSSDDKRPPTGRSPTRPGYSPERKPQDTRYRPSVSPEKKPELPLRPSASPERKPTERGSPTVSHRRVTSPTRLIGSRDSPIGSRESPERKHPSSSSNFPRSSPERNINNLPKDTARKPDLSPDRKPEHGKPHDGRKTPVTELPRTTSPPRKSHPKDSTNRVGSSPDRKPSYHPASPSSKTPDDIVEKQPKDALRTSVSPDRKPGYMKPTSASKPSDNLKSPTKHAPMQSPERKPMEKTTNKVQEDHYKFIDEETIMYTRNDKTKPKLPSEQKDHSPSRKSPSKDTPDFCNTELTRKRSPSPKDHVKFIDQKDRVDITQHKTVEVTEEITSKHRSPKEKDTPSDKASPKEPLPVNYGTYDKKKPFVSDVTEVTTTFRDSTDVKDDSQPRRQKSPNKTSEQPTSPTKKLPRDSVSPAKSPSKDSKHKHTTDFITTERTREEINKKTTEDRPRKLVTPSSSPTRKPKPTDTEPSTGQSSPTTSVSGFVYFSSPRTEKHIVTDLDDQEYYSEMHSTDETITNITKRPESLGVNRSQSPSKIPCRSPSPDKRTSPSKDSLPRKSSLKKPSTEVTQASPIEKPPSSFRVSPTEEPKDFPEHKVVKKDRPEGPESDAVVGKIKPPLERRETYEDRCRKILGMMDDTTTSETIKKTSYLKEPEANLSSPSVSPCRSPVPRETPFEYPTVKKTTQTKDDVTEFIVREQENIVKTSTVINDDTCINKAPRENLPVKLKDVTDKTTTKHERTTEDKVDTKKTVTNKYPSPRQSPERKHLNQKSEEDQKLYPDDLPRSTAGVEPTSKDSSKPRSTPLKPNTIGASPRSSISPERKPGYRPTEKKDDSKYPDKSTPATPGYMKTTSATSSKYDATTEENETIKTKTTIERKVTQSPTRKTPSDKPVGPDQRSDRSPQRKPSTDERAPIKTMPENKPSDTYPREASPSRPSYNKTTTSMIVEYDSTSTIEDIDQSMTSETVSKKSSPRPSVSPTRRVSKPSDDIPTRQSNKSPDRKHKDDLASTKDVPERKPTKPKDSSPSGPGYMKSTASMTSKYETTSTEDLRDTNTSKTVQTRYSSSPSRKTVKPSDGADSRPNHPKEQSPDRKPHAHQDRSPPRKLSENTPVDKYPRDSSPTKDKSDKIPGYMKTTSSVSSKYETISTTEEDVEENTSLTITKTNVRQPSDSPSKKELENYPRTSPPKSTTAKPLEGFSQTKPGRETAPKSGISGKPNTIEKTPEFVKTTSHIYKQDTVSATKDFLETCRTERQSPHHTPDSQTSKTSNEYGQETDHPRTVSPSRSHPTDYNSSRHSISPDRKSQQPSEPLVKKPTDEKRRGASPSKKIMNPETPGYMKTTTSVKTRHDEDFEEITSSKTRQSVHSLSRPSPPKTTKSDNAPSKPDLDRSPNRNLQKYTEPSPKASPERQIGYKYPKDTSPSRKTPASEETEPSNLKTITSIKSTLEDIKDTTTTKIKEGYHSTLHQSDTSPTRRGTKPTDSSKSTPDQSRTHSPTKSLPDASVKKSPAPKESINKYPRHSSPRSSPDRKTTTDSVVTCNTETISTTEEIETTEDTVSKHPSVGSPESPSRRAPYEPSKKRSYLQQPEETRQLRTPSPMKKTTKITEVSTDFLMSEREQEILDRVQKSLRKLSPDRKERSPSREKSPNKTTTSLKDIDIASHTLNDNEIINEDVIDITEQSTTKHGSLIAEKVKDSKVPTKLSPRTVSPTKKPSGIATPAKTDKISPESPTKPRSVSPKKPVSATERPQSPQVRPSGIKPKEPIPSHLTRKATSATLNITKTDKTVTDFKKITNIGSVTKQNSFSRSTTNKTTTKTISSPTSRPTEPETKRTPTPKGIRESVIPTKKDIDTKVMRTVSDITIKSKKTSPQRMKSKPEIQVSDMSTKTTKPSIKEPHSKLPSKPKSATTLNTSTDEDDIIIDVEQSKSSRENSPDRICPTPVNFGDDVGTPRYPDEVSEPDDDFRRRTHHTIHEAESIVDDIVEICEDDELFVRKTDVDVATEDDESLLSVTDKVSRFTKGVETVTKHKDTTAIFKDTERRTHSDFIDDKLKSDECLLSVSEKVNKFARGPSATRDSKSPSRRVTDEYDKNTVYQDDYTKLSVHDKAHLFVETAENAKTPKVKPTPQKVERPDLTNVDDSLKSDDCLLSVSDKVHKFVKTAEQFLTESQEVEEKEKRIKEQHDKIMRKIVDEVDDEVENYIEPTNVTEDVEVTSKKPSDRRDEKPHKPRETSSLLPKVKEYNSPSLKTADRTPTVKITTLRSSEAVKKAKALFETIATTTQKTKETIKTKTAKLTDIGVTKKSPKPDSTTDIYPCIEDSSPIYIPDKVGDEGDVPQTHKERPTSGTLSRPQYPRTDDKPRSSPSRLVAQSPEIPRSKSPGYQTVETTTTSKTVLSRYPTTQRAESPSQRHEPQKSDRIPGYLRPTKTSQIKEETKVVEDTEVSSRRGSGKFGVELRRTSVERSTASSERRRSVEHPCIEDIFDLDLLEQMLEKVVGYEQRRRIRSQIRIARKKVESDHIDTNTYTKTTRQTMTTQKLRSPERLRTRSPERAPKSTLHKAKSPDRHAKTPQRMIPETTRVPELAQPMHNGHAKEPTRTISDRRPRTHSPEKPAPKSVPKSKSPVRQPSPDKKTRPQSPNKTITPKPKPNRFNEYASAYMKKVGLSEADKVRFAEAKSRKAAEDRQRTIKHSEAHKVVEEYSTTKSFTSRTSSKDTIEVVQMNGTNKRSPSPEKRQSPERKAQSPERKAQSPERVYERTPSPTRKQVKSEPSKKETIIKTVYEIEKKMPQKSVQEEKPSWVTNRNLKKISSETRTFSSKKIEEKPKYRAPSPSKVITKPIDVITSSYGPGPLDADGKPLFGIRALRNGASNYQVKGTVIRQEYHSRNGGEPEGTVSVTAYSTEPEDLERLLQGQGEPPSRIHGLAAITTTKKFGGDTGTTLKEIYGREDRAAIDQFTHSDRRVSDSKVEEISQRSYDSRREDILGETRRIESRREEVIGDRLRQAREEIVDSAKDRREDRTERMQRRVEKVEKKGDRAQERKERIERTDDKKTVRQSSVKSLTEKYIKSANESSKTERAAYPKAGLILRTATMKDSVSSDSSAHAGLTRTDSEHSLGSVEDTVVTTERVDGGVRTVTTTTRSHGSVQRGDRSFLDSNTKVTGVQDILTRMKNADIVIQEGDTTEDTEARALLNKFLGATVLMAGMQGYVTEKPTGKLVVKQETVRTTSSGGGKVTSSQTSTLEDFDVDQCWDERLLRKLLDECNDYEQRRRLRARIRTLMAEQEACASAVTEALAAAGEADGADGDSREEEEVTVTSSVRRNSSETVSTSTTTTKTSKVIESMTRPAPKPVSPFAKFRQLEKQNSTNSPNSPQTPQSPGSPAQPYFKFTDPALQASAVTIKERLLQWCRDKTRDYENVKLENFSTSWADGLAFCALLHHFLPDAFDYSKLSPDKRRHNFTLAFKIADEKAGIYPLLDVDDMVTMRKPDWKCVFTYVQSIYRRFKDQN

SEQ ID NO: 64 - DNA, 13053 bp, synthetic construct
  source 1..13053: mol_type = other DNA
gcggccgcacagtgatcgctcggacgccgaccgcgccacacgccacgtacacccagctcgcatgctcgaagccgcaccacgcgaaaaaacacaataaaaccaatcgaccccgagtgaccccaaaagtgcataccgagcggccgacacgaacgaaacgacttcgcgaatacactctattcaaaaaactaacactaacgctttgcagtaacacgtgagctttattaactgttattataagttataattaacgtaaaggtggaaattaaactgtgcactggaagatatcaaagtttagagcgattgttttaaatttaaatgataacgggcgaggtgtgctagcggccggtggctgtgctcacacagaatgtctgaaggcgacgtgtcgttgatccgcgacgaggatctgctgcggcgcatgtggcagcagacggaagacttctctcgcaagaaagagattcgcgcgcacatgtaccgactgcgggaggagcgcctgcgcgacctgtactcgcccgaacctcccagggaggggaaaggtagtgagttcagtgctgcgcaaggccacgtgaagtcgttcgcggaccagagcttccagtccatgaagagcaaggaggtccgcgacgctggctccccacccaaggagttcacgtaccgcggccaagacttgaaggcgctgtcgaacgctggctggaatgtggagtcggagaacgagaccaccgacgatggtcacacgcacgtcaagtctgtccacgccaacattgaaggcagatacgatgtggatggcggcaaaggacaatttgctgccgtcgatcatcataaagaagccgtcaccgagtatcacgatgacaacagcagcttgaaacgtaacgagacctcgtcgaacacagcagctcgcgaacacgtcgtgcgcaaaactgatgacggcacgcagatatcgtctaccaccagctcgtcgacctcttcgtccaagttccagcagatgtcttcgacgcgacatgaaacagtgccatatttgactaacgatgactatgacctacgaagttatgatactaaccgcaatgaggattcatcctctgtaacgagaagacagatttcgaaaactaatgactacgaacaaaacttgaggaacaatgactatgaaacgaatttgagaaaaaatgactaccaacaaaatataataagaaacgaatatgaacaaaatgtcaaaaagaatgagtatgaacatgtcgtgaggaaaaatgattatgaacaaaatttgaccagaaatgattatgagactggcgagctcgtctctaggaaagtggattaccctgatgacaacaccagggtaattgtagagacgcggtgtttgcctgacggaactcgggttaccagcaccaagagagaatttcgggctccagtacaaagcactcgctccgaacattcataccagacaagggaaaataaatcctactcttcccaacagcgatctgatgcaaaggaatcctccaaaattatacgacacgttattgatgaccgcgcgactgacatcgtcgattcccaaagaaacgttgatgatcacgatttcaagagacaaattgctgaatacacttcaaaagacaataaagattattcggatgtgcagcgttatgaaacaaaagtgaacaagacaaatattgatcattctaagttggatgacgaatattcgaaaaccactcgccacgtgactcgagtcaataaagtaatagagtccacagataaagatgactatgtaaacacgcagtttacgaatcagacccatgaccgacaatatcaaattaatgatgttcgcaaggaaacggctgtccgcgagattcgccctgaaccaagcccgccgcgtgatcagcgcccacatacagttacaccgcaaaatgatcgacctaaatcttatacaccacgccacgatcgtcccgaaacgatgaccccagcggaaaaccgccgagacacagaaacacctaaaaacaatcgtcatgatataacatctccgcgcgataattaccgagagcctgtaaggcctcgtgatgaccagccggtaccggtgaaagattatgtccaacaccaaaccaaagataccgaggagagagtacatcacgtcactagagatcaaaagattgaggaaactgtagaaaggaagacaagctctgatcattaccaaaccacatatcaaacagattttcctcaaaaaaagatcagcaatgactggagtccatcacatcaggcgtgggctagtacactccgtgccgatacacctactaccacgcgaccttcaactagagcttcttctcctggtagtagaaccttcaagtccagctcatcttcgcttaggagcagcgttagccctgataaaatatccaggaagccatcgagcagaggtggtagtccaaacaaaattgacagacattcgccaacccgtaacgataaaaactctaccacacattctacccatagtgtcactgaggttaaaacgcataagtataccagttctgatgataaaagacctcccaccggtcgctctccaacgaggccgggttatagccctgagagaaaaccccaagacactcgctacaggcccagcgtaagtccagagaaaaaaccagaacttccattaaggcctagtgctagcccagaaaggaaaccaacagagagaggttcacctacagtgagccaccgtagagttacttctcctactcgcttaataggtagtcgagattcgccaataggatcgcgagaaagtccagagagaaagcacccttcttctagctcaaacttcccacgatcaagtccggaaagaaatattaataatttgcctaaggatactgcaaggaaaccagatctgagtcctgatagaaaaccagagcacggaaaacctcatgatggtcgtaaaactccagtgactgagttaccccgtaccacctctccacctcgaaagtctcaccctaaagatagcaccaacagagttggctcaagtccagaccgaaaacctagttatcatccagcaagtccttcgtcaaaaactcctgacgacattgttgaaaagcaaccaaaagatgccctaagaacaagtgtgagtcctgataggaaacctggatatatgaagccaacttctgcgagcaagccgtcagataacctcaagtccccaacgaagcatgcaccaatgcaaagccctgagcgtaagcctatggaaaaaactaccaacaaagtacaagaagatcactataaatttatcgatgaggaaacaataatgtacacccgtaacgataaaactaagccaaagttaccatctgaacaaaaagatcattcaccttcaaggaagagtcccagtaaagacactcccgatttctgtaacactgaattgacaagaaagagatcaccttctcccaaggatcatgttaaatttattgaccaaaaagatcgggttgatataacgcagcacaaaactgttgaggtaaccgaggaaataacgtcaaaacatcgttctccgaaggaaaaagatacacccagtgataaagcttctccaaaggagccattgccggttaattatggaacttatgacaaaaagaaaccgtttgtttctgatgttaccgaggtaactacaacttttagagacagtaccgatgtaaaggacgattcgcagccacgtcgtcaaaaatctccaaacaaaacatctgaacagccaacatctcccacaaagaagttgccacgagacagcgtgtcccctgctaaatctccttcaaaagattccaaacacaaacacactactgactttattacaacagagagaactagggaagagataaataaaaaaactactgaagatcgtccacgaaaactggttacaccatcctctagccccactaggaaaccgaaacctactgatactgaaccatcgactggtcaaagctctcctactacttctgtaagtggtttcgtatacttcagctcgccacgaactgaaaagcacattgtcactgacttagacgatcaagaatattattctgaaatgcactccaccgacgaaactattactaatatcactaaaagaccggaaagtctaggagtgaacagatcacaatctccttctaagattccttgcagatctccatctccagacaaacgcacctcgccttccaaggatagcctgccacgaaaaagttctttgaagaaaccctcaacagaagtaactcaagcttctccaattgaaaaacccccatccagttttcgtgtttcacccacagaagaaccaaaggacttccctgagcacaaagtggtaaaaaaagatcggccggaaggtcctgaatctgatgccgtcgttggcaaaataaaaccacctttagagcgtagagagacgtatgaggatcggtgccgaaaaattcttggaatgatggatgatacgactacatccgaaacgattaaaaaaaccagttatttgaaggagccagaagcaaacttgagctctcctagtgtctctccttgtcgcagtcctgttccaagagagactccatttgaatatccaacagttaagaaaacgacgcagactaaggacgatgtaaccgagttcatagtgcgtgaacaagaaaatattgtgaaaacaagcactgtaattaatgatgatacctgtattaataaagcacctcgggagaacttaccagtaaagctaaaagatgtgacagataaaactacaacaaaacatgaaagaacaactgaggataaagtcgatacaaagaaaactgtcaccaataagtatccttcaccgaggcaaagtcctgagaggaagcacttgaatcaaaagtcagaagaagatcagaaactgtatcctgacgaccttccgaggtcgactgcaggtgttgaacctacttccaaagactccagcaagcctcgttcaactccattgaaaccaaatacaataggtgcctcgccaagatctagcataagtcctgaaagaaaaccagggtatagaccgactgagaaaaaggatgacagtaaatatcctgacaaatctactccagcaacaccgggctatatgaaaaccacttcagctacttcatccaaatatgatgctacgactgaagagaatgaaacaattaagacaaaaacaacaattgaaagaaaagtaacacaatctccaacaagaaaaactccatctgataaaccagttggtccagatcaacgttctgacaggagtccccagcgaaaaccaagcactgacgaaagggcaccaatcaagacaatgcctgaaaataaaccttctgacacgtatcccagagaagcgtctccgtctagaccaagctacaataaaaccacaacttcaatgatagttgaatacgattccacctccacgattgaagatattgatcagtcgatgacttcagaaactgtatcaaaaaagtcatcccctcgtccttcagtatctccaacacgacgagtctcaaaaccatcagatgacattccaacaaggcagtccaacaagagtccagaccgcaagcataaagacgacctagcttctacaaaagatgttcctgagagaaaaccgacgaaaccaaaggactcctctccatcaggaccgggctacatgaaatctactgcttctatgacttcaaagtatgagaccacctccactgaagatctccgtgatactaatacttctaagacggtccaaacaagatactcatcttcaccctcacgcaaaactgttaaaccttctgatggcgctgatagcagacccaatcatcctaaagaacaaagccctgatcgcaagcctcacgctcatcaagatcgttctcctcctaggaaattatcagaaaacactcctgtagacaagtatcccagagatagttcaccaactaaagataaatctgataaaatccctggttatatgaaaactacttcgtctgtctcatcaaaatatgagactatatccaccactgaggaagatgtagaagaaaatacgtctttaactataactaaaaccaatgtgcggcagccatcagactctccatctaaaaaagaactggaaaactatcctcgtacttcaccacctaagtctactactgctaagccactggaaggcttttctcaaactaagcctggacgtgagacagcacctaaatcaggtatatccggcaagccaaacactattgagaaaactcctgagttcgtgaagacaacttctcatatttataaacaagatacagtgtccgccactaaagatttcttagaaacctgtagaacagaaaggcaaagtcctcatcatacccctgattcacaaacttcgaagacaagtaatgagtatggccaagaaaccgaccatcctcgaacagtttcaccatccaggtcacacccaacagattataactcatctagacatagcattagtcctgatcgtaagtctcagcagccttctgagccccttgtgaagaaaccaaccgatgaaaaacgcaggggagcatcgccttcaaagaaaattatgaatcccgaaactcctggttacatgaaaactactacgtctgttaaaactagacatgatgaggatttcgaagaaatcacatccagtaagaccagacagagtgtgcattcattatcacgtccatcaccacctaaaacgactaaatcagataacgctccaagtaaacccgatttggacagaagtcctaaccgcaacctacaaaaatacactgagccatcacccaaagctagcccagagagacaaataggttacaaatatcccaaggacacttctccgtcgcgaaaaacacctgcctcggaagagactgaacctagtaatttaaaaaccataacttctattaagtcaactttggaagatattaaggacaccacgactacaaaaatcaaagaaggataccattctactttgcatcagtcagacacttcaccaacaagaagaggcactaagcctacggatagttctaaatccactccggatcagtcgcgaacacattcaccaactaagtctctaccagatgcctctgtgaagaaatcacctgcacctaaagagagtattaacaaatatccacgacactcatctccaagatcaagtccagacagaaaaacaacaactgattcggttgtaacatgtaatactgaaactatttccaccaccgaagaaattgagactacagaagatacagtatccaagcatccatcagtaggatctcctgaatcgccttcacgccgtgctccttacgaaccatctaaaaagcgtagctaccttcagcaaccagaagagaccaggcagcttcgcacaccttcaccaatgaaaaaaactacaaaaattactgaagttagtacggacttcctgatgtccgagagagaacaggaaattttagatagagtccaaaagtcccttcgaaaattgtctcctgatcgtaaagagagatcacccagcagagaaaagagcccaaacaaaacaacaaccagtttgaaagacattgatatagctagccacactctgaacgacaatgaaattattaacgaagatgtaatagatatcacagagcaatcaaccactaaacatggatcgcttatagctgaaaaagtcaaggattccaaagtcccaaccaaattatctcccagaactgtttcgcctactaagaaaccttcaggtattgcaactccagcaaaaactgataaaataagtccggaatcacccacgaaaccaagaagtgtttcacccaaaaaaccagtttcagcaactgaaagaccacagtcaccgcaagtgcggccaagtggaataaaacctaaggagccgataccttctcatctgactagaaaggcgacgtctgccactttaaatatcaccaaaacggacaaaactgtaacagattttaagaaaattacaaatattggctctgtaactaaacaaaatagtttcagtcgttcaacaaccaataaaactactacaaagacaatctcttcacctacgagtcgacctaccgaaccagaaactaagcgaaccccaactcctaaaggcataagggagagcgtgatcccgacaaagaaagacatcgacacgaaagtgatgcggactgttagtgatattacaattaagtcgaagaaaacatcacctcaaagaatgaaatcaaagccagaaattcaagtgagtgatatgtctactaaaactacaaagccttctattaaagaaccacattctaaattgccaagtaaaccaaaatctgctacgacattgaatacttcaaccgacgaagatgacattattattgatgttgagcaatcgaaatcgtcgagagaaaattcacctgatcgtatttgtccaactcccgttaattttggagatgacgttggtacacctcgatatcctgatgaagtgagtgaacctgatgatgatttccgtagacgaactcatcatacaatccatgaggccgagtctatcgtggatgacattgttgaaatttgtgaagatgacgaactttttgttagaaagactgatgtcgacgttgcgactgaagatgacgaaagcctgcttagtgtaacagacaaagtatccagatttactaaaggtgttgagacagtaacgaagcataaagatacaactgcaatatttaaagatactgaaagaaggacacattctgatttcattgatgataagttgaaatctgatgagtgcttattgtcggtatcggagaaagtgaacaagtttgctagaggaccaagtgctactagagatagcaagagtccctcacggagagttaccgatgaatacgataaaaatactgtttatcaagatgactacactaaactaagtgtacatgacaaagcacacttgtttgtggagactgcagaaaacgctaagacacctaaagttaaaccaactccacaaaaagtggagcgtcctgacttaactaacgttgatgactcattaaagagtgatgattgcttactgagtgtttcggacaaagtacataaatttgtaaaaacagctgagcaattcttgacggaatctcaggaggtcgaggagaaggaaaagcgaattaaagagcaacatgacaaaatcatgcgcaagatagtagatgaagttgatgatgaggtagaaaactatatcgagccgacgaatgttaccgaagatgtggaagtcacaagcaaaaagccatcagaccgccgtgatgaaaagccccataaaccaagggaaacttcatcattgctaccgaaggttaaagagtacaacagccctagtctgaaaaccgctgatagaactccaaccgttaaaataactactcttcgcagtagtgaagctgttaagaaggctaaagctttgtttgaaacaatagcaaccactacgcaaaaaacaaaagaaaccattaaaactaaaactgctaaattaacagacatcggagtgacgaaaaagtctcctaaacctgattctactactgatatctatccctgtattgaagattcttcacctatttatattcctgataaagtgggggatgagggtgatgttcctcaaacccataaagagcgaccgacaagtggtacactcagccggccgcaatatcctcggactgatgataagccacggagcagcccgtcacgtttagtggcccagtctccagaaattccaaggtctaagtccccaggttatcaaactgtggaaaccacaactacatccaaaactgtgttatcaagatatccgactactcaaagagctgaatcaccgagtcagcgacacgagcctcaaaaatccgatagaattcctggataccttcgtccaacaaaaactagccaaatcaaggaggagacgaaggtggttgaggataccgaagtaagcagtcgacgagggagcggcaagtttggtgtagagcttcgaagaaccagtgttgaaagatcaacggcaagtagcgaacgccggcgaagcgtcgagcacccctgcatcgaagacatcttcgacctggacctcctggaacaaatgttggaaaaggtcgttggttacgagcagaggcgtcgcatcagatcacagattcggattgctagaaaaaaagtcgagagcgaccatattgacactaacacttacactaaaactacaagacaaactatgactacacaaaagctgcggtcaccggaacgcctgaggacgagatcacccgaacgggctccaaaatctaccctccataaagcaaaatcccctgatcgtcatgctaaaacaccccagagaatgatccccgaaaccacccgagtaccagaacttgcgcagcctatgcataatgggcacgctaaagagccaaccagaactataagtgacaggcgccccagaacacacagtcctgagaaacctgctccaaaatccgttcccaaatcaaaaagtccggttcgtcaacctagtcccgacaaaaagacacgacctcagtctcctaacaaaacaataactcctaaacccaaacccaatcgctttaatgaatatgcatcagcttacatgaagaaagtgggtttgagtgaggctgataaggtcagattcgccgaagcaaaatcaaggaaagcagcagaagacagacagagaaccatcaagcattcagaagcacataaagtagtggaagaatactccacaacgaaaagcttcacttcgaggacttcatctaaggataccatcgaagtggtgcagatgaatggaactaacaagaggtcaccgtcacctgaaaagcgtcagagtcctgaacgaaaggctcagagtccggaaagaaaggctcaaagcccggaacgagtttacgagcgcactccaagtcccacccgtaaacaggttaaatcagaaccttctaaaaaagagacaatcataaaaactgtttacgaaattgaaaagaagatgccgcagaaatcagttcaggaagagaagccgtcgtgggtgaccaacaggaacctcaagaagattagttcggaaaccagaacgttcagcagtaagaagatagaagagaagccaaagtacagagcgcccagcccctccaaggtgatcacgaagcctatcgacgtgatcacgtccagctacgggcccgggccgctcgacgctgacggcaaaccactcttcggtatcagggctttgaggaacggtgcatctaactatcaagtgaaaggcacggtgatccgccaagagtaccactcccgcaacggcggggagcccgagggcacagtctcggtgaccgcgtactccacggagcccgaagacctggagcgcctgctgcagggccagggggagcccccctcccgcatccacggcctggctgccattaccaccaccaagaagttcggcggagacacgggaacgacgctcaaggaaatttatgggagagaagatcgcgccgccatagaccagttcacacacagtgaccggcgcgtctccgactccaaggtcgaggagatctcccagaggagctacgactccagaagagaagatatcttgggggaaaccagacgtatcgagagcagacgggaggaggtcattggcgaccgcctccgtcaggctagggaggagatcgtggacagtgctaaagacaggcgggaagataggacggagaggatgcagaggcgagtggagaaagtggagaagaagggagatagggcgcaggagaggaaggagcggatagagaggacggatgataagaagactgtccgacagagctccgtcaagtcactcaccgagaaatacatcaagagtgccaacgagagctcgaagacggagcgcgccgcgtacccgaaggcggggctgatcctgcgcaccgccaccatgaaggacagcgtctccagcgactcctcggcacatgctggtctgacccgtaccgacagtgagcacagcctgggctcagtagaggacacggtggtgacgacggagcgtgtggacggaggagtcaggaccgtcaccaccaccaccaggagtcacggctccgtgcagcgcggggaccgctccttcctcgacagtaacacaaaggtcactggcgtccaggacatactcaccaggatgaagaatgctgatatagtgatccaggagggcgacacgacggaggacacggaggcgcgcgcgctgctcaacaagttcctgggcgccaccgtgctcatggccggcatgcagggctacgtcaccgagaagccgactggcaaactcgtcgtcaagcaggagacagttcgcaccacaagcagtggaggcggcaaggtcaccagctcgcagaccagcacccttgaggacttcgacgtcgaccagtgttgggacgagcgtcttctcaggaaactcttggacgagtgcaacgactacgagcaacgtcgacgactgcgcgcgcgcatccgaacactaatggcggaacaagaagcatgcgcgagcgccgtgacggaggcgctggcggcggcgggtgaagcggacggcgcggacggcgacagtcgcgaggaagaggaagtaactgtgacatcgagcgtgaggaggaactcgtcagaaactgttagcactagcactaccacaactaaaacatctaaagtgatagagagtatgaccaggccagcgcccaagcccgtgtcgccgttcgccaagttccggcagctcgaaaaacaaaattcaactaacagtcccaactcgccccagaccccgcagagcccgggctctccggcgcagccttacttcaagttcacggaccccgcgctgcaagccagcgccgtcactattaaagagcgattgctgcagtggtgtcgcgacaagacccgggactacgagaatgtgaagctggagaacttctcgacgagctgggcggacgggctggcgttctgcgcgctgctgcaccacttcctgcctgacgccttcgactactccaagctgtcgcctgacaagcgccgacacaacttcacgctcgccttcaaaattgccgatgagaaggcgggcatctacccgctgctggacgtggacgacatggtgacgatgcgcaagccggactggaagtgcgtgttcacgtacgtgcagtccatctaccgccggttcaaggaccagaactgacgtcacccggtcgcccagccgtccagcctcccactgaacaccgcctg

SEQ ID NO: 65 - DNA, 300 bp, synthetic construct
  source 1..300: mol_type = other DNA
cagggaggggaaaggtagtgagttcagtgctgcgcaaggccacgtgaagtcgttcgcggaccagagcttccagtccatgaagagcaaggaggtccgcgacgctggctccccacccaaggagttcacgtaccgcggccaagacttgaaggcgctgtcgaacgctggctggaatgtggagtcggagaacgagaccaccgacgatggtcacacgcacgtcaagtctgtccacgccaacattgaaggcagatacgatgtggatggcggcaaaggacaatttgctgccgtcgatcatcataaaga

SEQ ID NO: 66 - DNA, 300 bp, synthetic construct
  source 1..300: mol_type = other DNA
tagggaggggaaaggtagtgagtttagtgttgtgtaaggttatgtgaagttgtttgtggattagagtttttagtttatgaagagtaaggaggtttgtgatgttggttttttatttaaggagtttatgtattgtggttaagatttgaaggtgttgttgaatgttggttggaatgtggagttggagaatgagattattgatgatggttatatgtatgttaagtttgtttatgttaatattgaaggtagatatgatgtggatggtggtaaaggataatttgttgttgttgattattataaaga

SEQ ID NO: 67 - DNA, 793 bp, synthetic construct
  source 1..793: mol_type = other DNA
aactcgagtaatacgactcactatagggtctttatgatgatcgacggcagcaaattgtcctttgccgccatccacatcgtatctgccttcaatgttggcgtggacagacttgacgtgcgtgtgaccatcgtcggtggtctcgttctccgactccacattccagccagcgttcgacagcgccttcaagtcttggccgcggtacgtgaactccttgggtggggagccagcgtcgcggacctccttgctcttcatggactggaagctctggtccgcgaacgacttcacgtggccttgcgcagcactgaactcactacctttcccctccctgaacttggacgaagaggtcgacgagctggtggtagacgatatctgcgtgccgtcatcagttttgcgcacgacgtgttcgcgagctgctgtgttcgacgaggtctcgttacgtttcaagctgctgttgtcatcgtgatactcggtgacggccagggaggggaaaggtagtgagttcagtgctgcgcaaggccacgtgaagtcgttcgcggaccagagcttccagtccatgaagagcaaggaggtccgcgacgctggctccccacccaaggagttcacgtaccgcggccaagacttgaaggcgctgtcgaacgctggctggaatgtggagtcggagaacgagaccaccgacgatggtcacacgcacgtcaagtctgtccacgccaacattgaaggcagatacgatgtggatggcggcaaaggacaatttgctgccgtcgatcatcataaagacccgggcgtctagaga

SEQ ID NO: 68 - DNA, 793 bp, synthetic construct
  source 1..793: mol_type = other DNA
aactcgagtaatacgactcactatagggtctttatgatgatcgacggcagcaaattgtcctttgccgccatccacatcgtatctgccttcaatgttggcgtggacagacttgacgtgcgtgtgaccatcgtcggtggtctcgttctccgactccacattccagccagcgttcgacagcgccttcaagtcttggccgcggtacgtgaactccttgggtggggagccagcgtcgcggacctccttgctcttcatggactggaagctctggtccgcgaacgacttcacgtggccttgcgcagcactgaactcactacctttcccctccctgaacttggacgaagaggtcgacgagctggtggtagacgatatctgcgtgccgtcatcagttttgcgcacgacgtgttcgcgagctgctgtgttcgacgaggtctcgttacgtttcaagctgctgttgtcatcgtgatactcggtgacggctagggaggggaaaggtagtgagtttagtgttgtgtaaggttatgtgaagttgtttgtggattagagtttttagtttatgaagagtaaggaggtttgtgatgttggttttttatttaaggagtttatgtattgtggttaagatttgaaggtgttgttgaatgttggttggaatgtggagttggagaatgagattattgatgatggttatatgtatgttaagtttgtttatgttaatattgaaggtagatatgatgtggatggtggtaaaggataatttgttgttgttgattattataaagacccgggcgtctagaga

SEQ ID NO: 69 - DNA, 20 bp, synthetic construct
  source 1..20: mol_type = other DNA
cagcgagtggagctcacagc

SEQ ID NO: 70 - DNA, 23 bp, synthetic construct
  source 1..23: mol_type = other DNA
actgtagcttcccnggggagaag

SEQ ID NO: 71 - DNA, 24 bp, synthetic construct
  source 1..24: mol_type = other DNA
ggaagtaatcaagacgaaggaacc

SEQ ID NO: 72 - DNA, 20 bp, synthetic construct
  source 1..20: mol_type = other DNA
gggccgtctttctcgaagta

SEQ ID NO: 73 - DNA, 21 bp, synthetic construct
  source 1..21: mol_type = other DNA
ttgtcatcttctcagaccggc

SEQ ID NO: 74 - DNA, 20 bp, synthetic construct
  source 1..20: mol_type = other DNA
cccgtagcgtgttcaggtaa

SEQ ID NO: 75 - DNA, 20 bp, synthetic construct
  source 1..20: mol_type = other DNA
gttcccggagtacctgcaat

SEQ ID NO: 76 - DNA, 20 bp, synthetic construct
  source 1..20: mol_type = other DNA
aagccgttcttctctgcctc

SEQ ID NO: 77 - DNA, 21 bp, synthetic construct
  source 1..21: mol_type = other DNA
ggtcactacaatggctacccg

SEQ ID NO: 78 - DNA, 20 bp, synthetic construct
  source 1..20: mol_type = other DNA
ggctgtatgcctcccatgtt

SEQ ID NO: 79 - DNA, 20 bp, synthetic construct
  source 1..20: mol_type = other DNA
gactcactcttcccttgcgt

SEQ ID NO: 80 - DNA, 20 bp, synthetic construct
  source 1..20: mol_type = other DNA
tcgcttcatgatggactccg

SEQ ID NO: 81 - DNA, 22 bp, synthetic construct
  source 1..22: mol_type = other DNA
tagacgaggacacagatgtgga

SEQ ID NO: 82 - DNA, 26 bp, synthetic construct
  source 1..26: mol_type = other DNA
ggaatggaacggtgagggttataaca

SEQ ID NO: 83 - DNA, 20 bp, synthetic construct
  source 1..20: mol_type = other DNA
ggaagccgacatttgcatgg

SEQ ID NO: 84 - DNA, 22 bp, synthetic construct
  source 1..22: mol_type = other DNA
ggagaatgttgcgaacacaagg

SEQ ID NO: 85 - DNA, 20 bp, synthetic construct
  source 1..20: mol_type = other DNA
caacactcgcactcggattc

SEQ ID NO: 86 - DNA, 20 bp, synthetic construct
  source 1..20: mol_type = other DNA
atcgtcagtgtccatggtgt

SEQ ID NO: 87 - DNA, 20 bp, synthetic construct
  source 1..20: mol_type = other DNA
atcagggctttgaggaacgg

SEQ ID NO: 88 - DNA, 20 bp, synthetic construct
  source 1..20: mol_type = other DNA
gcgcgatcttctctcccata

SEQ ID NO: 89 - DNA, 21 bp, synthetic construct
  source 1..21: mol_type = other DNA
gacaaacgtaccatcgagaag

SEQ ID NO: 90 - DNA, 20 bp, synthetic construct
  source 1..20: mol_type = other DNA
gataccagcctcgaagtcac

SEQ ID NO: 91 - DNA, 332 bp, synthetic construct
  source 1..332: mol_type = other DNA
cactctgtggtctcaggaggttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgtacttgagaccacgaagtg

SEQ ID NO: 92 - DNA, 877 bp, synthetic construct
  source 1..877: mol_type = other DNA
cactctgtggtctcatactgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgtgagaccacgaagtg

SEQ ID NO: 93 - DNA, 1779 bp, synthetic construct
  source 1..1779: mol_type = other DNA
cactctgtggtctcagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagataatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtcctggttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggagaagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgtggttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgcgcttgagaccacgaagtg

SEQ ID NO: 94 - DNA, 825 bp, synthetic construct
  source 1..825: mol_type = other DNA
cactctgtggtctcaaatgcgtcgtaccgccgagcgtcgtaccgccgagccactcgcaacatcatcacctcacttgagcacgcctttaataacgtgatctgatctgactgcgagatcttggcgaagcccggtaggcctttagcgaattctacgatgagctgcactgtgagaatcgtcatctcggtaatctgacggaacggcatatccgagtcctcgtcgtcctcgtccgactgccaggtctgtgtaaccctcttcaggtcttcctcggaaggttgttcgtagccttcctggtaccacacgagccttgcgatcaacgacttctgattggcagtgagcagcgcatagtgcacgttgtccatcatcatggagtacatgcaccgacagaagtgcagcaggtcttcgatgacgtacgccatgcctgccttgcggtagttgtcgcgagtgtaggcctggttgttggcgaacagcacgctgtcggtagccgttattgttaattagaagttgttgattgtaaggtttgtgtggtattaggaaggttatgaataattttttgaggaagatttgaagagggttatatagatttggtagttggatgaggatgatgaggatttggatatgttgttttgttagattattgagatgatgatttttatagtgtagtttattgtagaatttgttaaaggtttattgggttttgttaagattttgtagttagattagattatgttattaaaggtgtgtttaagtgaggtgatgatgttgtgagtggtttggtggtatgatgtgagtggtttggtggtatgatggctttgagaccacgaagtg

SEQ ID NO: 95 - DNA, 191 bp, synthetic construct
  source 1..191: mol_type = other DNA
tcgcgtcggcatcggtcagtggcagtgaagggcgacagttcctgattaaccacaaccgttctactttactggcttggtcgtcatgaagatgcggactgcgtggcaaaggattcgatacgtgctgatggtgcacgaccagcattaatggactggattgggccaactcctaccgtacctcgcttacccttacg

SEQ ID NO: 96 - DNA, 183 bp, synthetic construct
  source 1..183: mol_type = other DNA
tcgcgtcggctccggtcagtgcagtgaaggcgaacagttctgattaaccaaaaccgttctctttactggcttggtcgtcagaagatgcggcttgcgtggcaaggattcgaaacgtgctgaggtgcacgacacgcattaatgactggattgggccaactccaccgtacctccattacccttacg

SEQ ID NO: 97 - DNA, 177 bp, synthetic construct
  source 1..177: mol_type = other DNA
tcgcgtcggcaccggtcatggcagtaagggcaacagttctgattaccacaaccgttctctttactgctttggcgtcataagatgcgacttgctggcaaggattcgaaacgtgcgatggtgacgacccgcattatggactgattgggccaactctaccgtactcgcattacccttacg

SEQ ID NO: 98 - DNA, 176 bp, synthetic construct
  source 1..176: mol_type = other DNA
tcgcgtcggtccggtcagtgcagtgaagggaacagttcctgttaaccacaacgttctacttactggctttggttcatgaagatcggacttgcggcaaaggattgataacgtgcatggtgcacgccacgcattaggactggattgggccaactcaccgtacctccattacccttacg

SEQ ID NO: 99 - DNA, 176 bp, synthetic construct
  source 1..176: mol_type = other DNA
tcgcgtcggcatccgagtggcagtgaagggcgaacacctgattaaccacaaaccgttctttactggctttggtcgtcaagatgcggacttgcgtggcaaattcgataacgtgctgatggtcgaccacgcattaatggactgtggggccaactcctaccgtacgcattacccttacg

SEQ ID NO: 100 - DNA, 1235 bp, synthetic construct
  source 1..1235: mol_type = other DNA
ctcgaggatcctcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgaagcttggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 101 - DNA, 468 bp, synthetic construct
  source 1..468: mol_type = other DNA
ctcgaggatcctcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgaagcttggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 102 - DNA, 457 bp, synthetic construct
  source 1..457: mol_type = other DNA
ctcgagttgtgttggtatttggttagtggtagtgaagggtgaatagtttttgattaattataaattgttttattttattggttttggttgttatgaagatgtggatttgtgtggtaaaggatttgataatgtgttgatggtgtatgattatgtattaatggattggattggggttaatttttattgtattttgtattatttttatgggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 103 - DNA, 448 bp, synthetic construct
  source 1..448: mol_type = other DNA
ctcgagtcgcgtcggcatcggtcagtggcagtgaagggcgacagttcctgattaaccacaaccgttctactttactggcttggtcgtcatgaagatgcggactgcgtggcaaaggattcgatacgtgctgatggtgcacgaccagcattaatggactggattgggccaactcctaccgtacctcgcttacccttacgggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 104 - DNA, 440 bp, synthetic construct
  source 1..440: mol_type = other DNA
ctcgagtcgcgtcggctccggtcagtgcagtgaaggcgaacagttctgattaaccaaaaccgttctctttactggcttggtcgtcagaagatgcggcttgcgtggcaaggattcgaaacgtgctgaggtgcacgacacgcattaatgactggattgggccaactccaccgtacctccattacccttacgggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 105 - DNA, 434 bp, synthetic construct
  source 1..434: mol_type = other DNA
ctcgagtcgcgtcggcaccggtcatggcagtaagggcaacagttctgattaccacaaccgttctctttactgctttggcgtcataagatgcgacttgctggcaaggattcgaaacgtgcgatggtgacgacccgcattatggactgattgggccaactctaccgtactcgcattacccttacgggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 106 - DNA, 433 bp, synthetic construct
  source 1..433: mol_type = other DNA
ctcgagtcgcgtcggtccggtcagtgcagtgaagggaacagttcctgttaaccacaacgttctacttactggctttggttcatgaagatcggacttgcggcaaaggattgataacgtgcatggtgcacgccacgcattaggactggattgggccaactcaccgtacctccattacccttacgggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 107 - DNA, 433 bp, synthetic construct
  source 1..433: mol_type = other DNA
ctcgagtcgcgtcggcatccgagtggcagtgaagggcgaacacctgattaaccacaaaccgttctttactggctttggtcgtcaagatgcggacttgcgtggcaaattcgataacgtgctgatggtcgaccacgcattaatggactgtggggccaactcctaccgtacgcattacccttacgggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 108 - DNA, 4851 bp, synthetic construct
  source 1..4851: mol_type = other DNA
atctctctctttcgatggaactgagctctttctctctttcctcttcttttctctctctatctctatctctcgtagcttgataagagtttctctcttttgaagatccgtttctctctctctcactgagactattgttgttaggtcaacttgcgatcatggcgatttcgaaggtctgaagctgatttcgaatggtttggagatatccgtagtggttaagcatatggaagtctatgttctgctcttggttgctctgttagggcttcctccatttggaccaacttagctgaatgttgtatgatctctctccttgaagcagcaaataagaagaaggtctggtccttaacttaacatctggttactagaggaaacttcagctattattaggtaaagaaagactgtacagagttgtataacaagtaagcgttagagtggctttgtttgcctcggtgatagaagaaccgactgattcgttgttgtgtgttagctttggagggaatcagatttcgcgagggaaggtgttttagatcaaatctgtgaattttactcaactgaggcttttagtgaaccacgactgtagagttgaccttgaatcctactctgagtaattatattatcagatagatttaggatggaagctgaaattgtgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttgtgactggtaaacacttggctcagatctgcaatgaagaatatgacaagtggacgtgcatgttcttgggcattcaggcggagttctcagcaattctgctcgaccttaccatggttgtgggagttgcgcatgcacttaaccttttgtttggggtggagttatccactggagtgtttttggccgccatggatgcgtttttatttcctgttttcgcctctttccttgaaaatggtatggcaaatacagtatccatttactctgcaggcctggtattacttctctatgtatctggcgtcttgctgagtcagtctgagatcccactctctatgaatggagtgttaactcggttaaatggagagagcgcattcgcactgatgggtcttcttggcgcaagcatcgtccctcacaatttttatatccattcttattttgctggggaaagtacatcttcgtctgatgtcgacaagagcagcttgtgtcaagaccatttgttcgccatctttggtgtcttcagcggactgtcacttgtaaattatgtattgatgaatgcagcagctaatgtgtttcacagtactggccttgtggtactgacttttcacgatgccttgtcactaatggagcaggtatttatgagtccgctcattccagtggtctttttgatgctcttgttcttctctagtcaaattaccgcactagcttgggctttcggtggagaggtcgtcctgcatgacttcctgaagatagaaatacccgcttggcttcatcgtgctacaatcagaattcttgcagttgctcctgcgctttattgtgtatggacatctggtgcagacggaatataccagttacttatattcacccaggtcttggtggcaatgatgcttccttgctcggtaataccgcttttccgcattgcttcgtcgagacaaatcatgggtgtccataaaatccctcaggttggcgagttcctcgcacttacaacgtttttgggatttctggggttgaatgttgtttttgttgttgagatggtatttgggagcagtgactgggctggtggtttgagatggaataccgtgatgggcacctcgattcagtacaccactctgcttgtatcgtcatgtgcatccttatgcctgatactctggctggcagccacgccgctgaaatctgcgagtaacagagcggaagctcaaatatggaacatggatgctcaaaatgctttatcttatccatctgttcaagaagaggaaattgaaagaacagaaacaaggaggaacgaagacgaatcaatagtgcggttggaaagcagggtaaaggatcagttggatactacgtctgttactagctcggtctatgatttgccagagaacattctaatgacggatcaagaaatccgttcgagccctccagaggaaagagagttggatgtaaagtactctacctctcaagttagtagtcttaaggaagactctgatgtaaaggaacagtctgtattgcagtcaacagtggttaatgaggtcagtgataaggatctgattgttgaaacaaagatggcgaaaattgaaccaatgagtcctgtggagaagattgttagcatggagaataacagcaagtttattgaaaaggatgttgaaggggtttcatgggaaacagaagaagctaccaaagctgctcctacaagcaactttactgtcggatctgatggtcctccttcattccgcagcttaagtggggaagggggaagtgggactggaagcctttcacggttgcaaggtttgggacgtgctgcccggagacacttatctgcgatccttgatgaattttggggacatttatatgattttcatgggcaattggttgctgaagccagggcaaagaaactagatcagctgtttggcactgatcaaaagtcagcctcttctatgaaagcagattcgtttggaaaagacattagcagtggatattgcatgtcaccaactgcgaagggaatggattcacagatgacttcaagtttatatgattcactgaagcagcagaggacaccgggaagtatcgattcgttgtatggattacaaagaggttcgtcaccgtcaccgttggtcaaccgtatgcagatgttgggtgcatatggtaacaccactaataataataatgcttacgaattgagtgagagaagatactctagcctgcgtgctccatcatcttcagagggttgggaacaccaacaaccagctacagttcacggataccagatgaagtcatatgtagacaatttggcaaaagaaaggcttgaagccttacaatcccgtggagagatcccgacatcgagatctatggcgcttggtacattgagctatacacagcaacttgctttagccttgaaacagaagtcccagaatggtctaacccctggaccagctcctgggtttgagaattttgctgggtctagaagcatatcgcgacaatctgaaagatcttattacggtgttccatcttctggcaatactgatactgttggcgcagcagtagccaatgagaaaaaatatagtagcatgccagatatctcaggattgtctatgtccgcaaggaacatgcatttaccaaacaacaagagtggatactgggatccgtcaagtggaggaggagggtatggtgcgtcttatggtcggttaagcaatgaatcatcgttatattctaatttggggtcacgggtgggagtaccctcgacttatgatgacatttctcaatcaagaggaggctacagagatgcctacagtttgccacagagtgcaacaacagggaccggatcgctttggtccagacagccctttgagcagtttggtgtagcggagaggaatggtgctgttggtgaggagctcaggaatagatcgaatccgatcaatatagacaacaacgcttcttctaatgttgatgcagaggctaagcttcttcagtcgttcaggcactgtattctaaagcttattaaacttgaaggatccgagtggttgtttggacaaagcgatggagttgatgaagaactgattgaccgggtagctgcacgagagaagtttatctatgaagctgaagctcgagaaataaaccaggtgggtcacatgggggagccactaatttcatcggttcctaactgtggagatggttgcgtttggagagctgatttgattgtgagctttggagtttggtgcattcaccgtgtccttgacttgtctctcatggagagtcggcctgagctttggggaaagtacacttacgttctcaaccgcctacagggagtgattgatccggcgttctcaaagctgcggacaccaatgacaccgtgcttttgccttcagattccagcgagccaccagagagcgagtccgacttcagctaacggaatgttacctccggctgcaaaaccggctaaaggcaaatgcacaaccgcagtcacacttcttgatctaatcaaagacgttgaaatggcaatctcttgtagaaaaggccgaaccggtacagctgcaggtgatgtggctttcccaaaggggaaagagaatttggcttcggttttgaagcggtataaacgtcggttatcgaataaaccagtaggtatgaatcaggatggacccggttcaagaaaaaacgtgactgcgtacggatcattgggttgaagaagaagaacattgtgagaaatctcatgatcaaagtgacgtcgagagggaagccgaagaatcaaaactctcgcttttgattgctcctctgcttcgttaattgtgtattaagaaaagaagaaaaaaaatggatttttgttgcttcagaatttttcgctctttttttcttaatttggttgtaatgttatgtttatatacatatatcatcatcataggaccatagctacaaaccgaatccggtttgtgtaattctatgcggaatcataaagaaatcgtcggtttgaaatgttaaatctcctaaaccggatctctgcacgtagctgacacatcgacgctag

SEQ ID NO: 109 - DNA, 1703 bp, synthetic construct
  source 1..1703: mol_type = other DNA
gttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccaaatacacctaacttgtttagtacacaacagcaacatcaaactctaataaacccaagttggtgtatactataatggtgatggctggtgcttcttctttggatgagatcagacaggctcagagagctgatggacctgcaggcatcttggctattggcactgctaaccctgagaaccatgtgcttcaggcggagtatcctgactactacttccgcatcaccaacagtgaacacatgaccgacctcaaggagaagttcaagcgcatgtgcgacaagtcgacaattcggaaacgtcacatgcatctgacggaggaattcctcaaggaaaacccacacatgtgtgcttacatggctccttctctggacaccagacaggacatcgtggtggtcgaagtccctaagctaggcaaagaagcggcagtgaaggccatcaaggagtggggccagcccaagtcaaagatcactcatgtcgtcttctgcactacctccggcgtcgacatgcctggtgctgactaccagctcaccaagcttcttggtctccgtccttccgtcaagcgtctcatgatgtaccagcaaggttgcttcgccggcggtactgtcctccgtatcgctaaggatctcgccgagaacaatcgtggagcacgtgtcctcgttgtctgctctgagatcacagccgttaccttccgtggtccctctgacacccaccttgactccctcgtcggtcaggctcttttcagtgatggcgccgccgcactcattgtggggtcggaccctgacacatctgtcggagagaaacccatctttgagatggtgtctgccgctcagaccatccttccagactctgatggtgccatagacggacatttgagggaagttggtctcaccttccatctcctcaaggatgttcccggcctcatctccaagaacattgtgaagagtctagacgaagcgtttaaacctttggggataagtgactggaactccctcttctggatagcccaccctggaggtccagcgatcctagaccaggtggagataaagctaggactaaaggaagagaagatgagggcgacacgtcacgtgttgagcgagtatggaaacatgtcgagcgcgtgcgttctcttcatactagacgagatgaggaggaagtcagctaaggatggtgtggccacgacaggagaagggttggagtggggtgtcttgtttggtttcggaccaggtctcactgttgagacagtcgtcttgcacagcgttcctctctaaacagaacgcttgccttctatctgcctacctacctacgcaaaactttaatcctgtcttatgttttatataatataatcattatatgtttacgcaataattaaggaagaattcatttgatgtgatatgtgatatgtgctggacaggtctattcgactgtttttgtactctcttttttgtgtctttttacaatattaaatctatgggtcttgaatgacatcaaatctttgtt

SEQ ID NO: 110 - DNA, 200 bp, synthetic construct
  source 1..200: mol_type = other DNA
gacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgtt

SEQ ID NO: 111 - DNA, 191 bp, synthetic construct
  source 1..191: mol_type = other DNA
gacctcagctaggtttatccagagaatggttcctctctacttcctgtccttttggttctgtcggatatattgatccgggaaatgggttgcaaatatcaaggaggtgctcgtttcgggttgacttggtggcaattactctcttttcaattttgccgccatctatgccaatatgttgcagctccataagcgtt

SEQ ID NO: 112 - DNA, 183 bp, synthetic construct
  source 1..183: mol_type = other DNA
gacctcgctagggtttaccagagaatgttcctgctctcttcctgtcctttggtttcttcggatatatgatcccgggaatgggttgcaatatcgaaggggtgctcgttcgggtatgactggtggcaatactctgctttcaattttgccccatcttatgcaatatgttgagctcgcataagcgtt

SEQ ID NO: 113 - DNA, 177 bp, synthetic construct
  source 1..177: mol_type = other DNA
gacctcagctaggtttatcagagaatgttcctctctactcctgtcctttggttctgtcgatatatgatcccggaaatggttgcaatatcgaagaggtgtcgtttcggtatgattggtgcaattacctgctttcaatttgccgccacttatgcaatatgttcagctcgcataagcgtt

SEQ ID NO: 114 - DNA, 176 bp, synthetic construct
  source 1..176: mol_type = other DNA
gacctcagctagtttatccaggaatggttccctctacttccgtccttttggtctgtcggattattgatcccgaaatgggttcaaatatcgagaggtgctcgttcgggtatgttggtggcaatactctgcttcaattttgccccatcttatgaatatgttgcgctcgcataagcgtt

SEQ ID NO: 115 - DNA, 176 bp, synthetic construct
  source 1..176: mol_type = other DNA
gacctcagctgtttatccagagaatggttcctctacttcctgtccttttggttgtcggatatattgatcccgggagggttgcaaatatcgaaggagctcgtttcgggtatgacttggcaattactctgcttttcaattccgccatcttatgccaatatgcagctcgcataagcgtt

SEQ ID NO: 116 - DNA, 200 bp, synthetic construct
  source 1..200: mol_type = other DNA
gttgtctgctctgagatcacagccgttaccttccgtggtccctctgacacccaccttgactccctcgtcggtcaggctcttttcagtgatggcgccgccgcactcattgtggggtcggaccctgacacatctgtcggagagaaacccatctttgagatggtgtctgccgctcagaccatccttccagactctgatggtgc

SEQ ID NO: 117 - DNA, 600 bp, synthetic construct
  source 1..600: mol_type = other DNA
cgaactccaactggctcgtatggtggaggcgaagaagcagagacgaaggtctcacaaacagaatcgacgggaacgaggtcacaaaagtcccagcgagagagcgcgttcaaatctcagactattccgcttcctaccgttctatcaagtggatggttcggaactgacagggtcatgccgccatgtgaacgtggcggagttacccgagtctgaggcctctcgtttagagttatcggcggaagaccatgattttgacgatacagattggttcgccggtaacgaatgggcggaaggtgctttcccgactgggccagctgtagaagtcgatgtgaaggacataccagcacaggaggtggagcatgaagctaaatcgctcatgagaggcttgagagacttcaacccaattgcccaaacagtttgtaggctgaaagtatctgttgaatatggggcatcagagatgtacggttttggatttggagcatacatagtagcgaaccaccatttatttaggagttacaatggttccatggaggtgcaatccatgcacggtacattcagggtgaagaatctacacagtttgagcgttctgccaattaaaggtag

SEQ ID NO: 118 - DNA, 1493 bp, synthetic construct
  source 1..1493: mol_type = other DNA
ctcgaggaattcggtaccccaattctaatacgactcactatagggcgaactccaactggctcgtatggtggaggcgaagaagcagagacgaaggtctcacaaacagaatcgacgggaacgaggtcacaaaagtcccagcgagagagcgcgttcaaatctcagactattccgcttcctaccgttctatcaagtggatggttcggaactgacagggtcatgccgccatgtgaacgtggcggagttacccgagtctgaggcctctcgtttagagttatcggcggaagaccatgattttgacgatacagattggttcgccggtaacgaatgggcggaaggtgctttcccgactgggccagctgtagaagtcgatgtgaaggacataccagcacaggaggtggagcatgaagctaaatcgctcatgagaggcttgagagacttcaacccaattgcccaaacagtttgtaggctgaaagtatctgttgaatatggggcatcagagatgtacggttttggatttggagcatacatagtagcgaaccaccatttatttaggagttacaatggttccatggaggtgcaatccatgcacggtacattcagggtgaagaatctacacagtttgagcgttctgccaattaaaggtagctgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagctacctttaattggcagaacgctcaaactgtgtagattcttcaccctgaatgtaccgtgcatggattgcacctccatggaaccattgtaactcctaaataaatggtggttcgctactatgtatgctccaaatccaaaaccgtacatctctgatgccccatattcaacagatactttcagcctacaaactgtttgggcaattgggttgaagtctctcaagcctctcatgagcgatttagcttcatgctccacctcctgtgctggtatgtccttcacatcgacttctacagctggcccagtcgggaaagcaccttccgcccattcgttaccggcgaaccaatctgtatcgtcaaaatcatggtcttccgccgataactctaaacgagaggcctcagactcgggtaactccgccacgttcacatggcggcatgaccctgtcagttccgaaccatccacttgatagaacggtaggaagcggaatagtctgagatttgaacgcgctctctcgctgggacttttgtgacctcgttcccgtcgattctgtttgtgagaccttcgtctctgcttcttcgcctccaccatacgagccagttggagttcgccctatagtgagtcgtattagaattgggttcgaaatcgataagcttggatcc

SEQ ID NO: 119 - DNA, 574 bp, synthetic construct
  source 1..574: mol_type = other DNA
cgaactccaactggctcgtatgtggaggcgaagaagcagagcgaaggtctcacaaacagaacgacgggaacgaggtcacaaagtcccagcgagagagcgcgtcaaatctcagactattccgctcctaccgttctatcaagtgatggttcggaactgacagggtatgccgccatgtgaacgtggcgagttacccgagtctgaggctctcgtttagagttatcggcgaagaccatgattttgacgaacagattggttcgccggtaagaatgggcggaaggtgctttcccgactgggccagctgtagaagtgatgtgaaggacataccagcaaggaggtggagcatgaagctaatcgctcatgagaggcttgaagacttcaacccaattgcccaacagtttgtaggctgaaagatctgttgaatatggggcatagagatgtacggttttggattggagcatacatagtagcgaacaccatttatttaggagttacatggttccatggaggtgcaaccatgcacggtacattcaggtgaagaatctacacagtttgaggttctgccaattaaaggtag

SEQ ID NO: 120 - DNA, 1416 bp, synthetic construct
  source 1..1416: mol_type = other DNA
ctcgaggaattctaatacgactcactatagggcgaactccaactggctcgtatgtggaggcgaagaagcagagcgaaggtctcacaaacagaacgacgggaacgaggtcacaaagtcccagcgagagagcgcgtcaaatctcagactattccgctcctaccgttctatcaagtgatggttcggaactgacagggtatgccgccatgtgaacgtggcgagttacccgagtctgaggctctcgtttagagttatcggcgaagaccatgattttgacgaacagattggttcgccggtaagaatgggcggaaggtgctttcccgactgggccagctgtagaagtgatgtgaaggacataccagcaaggaggtggagcatgaagctaatcgctcatgagaggcttgaagacttcaacccaattgcccaacagtttgtaggctgaaagatctgttgaatatggggcatagagatgtacggttttggattggagcatacatagtagcgaacaccatttatttaggagttacatggttccatggaggtgcaaccatgcacggtacattcaggtgaagaatctacacagtttgaggttctgccaattaaaggtagctgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagctacctttaattggcagaacgctcaaactgtgtagattcttcaccctgaatgtaccgtgcatggattgcacctccatggaaccattgtaactcctaaataaatggtggttcgctactatgtatgctccaaatccaaaaccgtacatctctgatgccccatattcaacagatactttcagcctacaaactgtttgggcaattgggttgaagtctctcaagcctctcatgagcgatttagcttcatgctccacctcctgtgctggtatgtccttcacatcgacttctacagctggcccagtcaaagcaccttccgcccattcgttaccggcgaaccaatctgtatcgtcaaaatcatggtcttccgccgataactctaaacgagaggcctcagactcgggtaactccgccacgttcacatggcggcatgaccctgtcagttccgaaccatccacttgatagaacggtaggaagcggaatagtctgagatttgaacgcgctctctcgctgggacttttgtgacctcgttcccgtcgattctgtttgtgagaccttcgtctctgcttcttcgcctccaccatacgagccagttggagttcgcccaagctttctagaaa

SEQ ID NO: 121 - DNA, 2653 bp, synthetic construct
  source 1..2653: mol_type = other DNA
agaaaaatcgaaaaatgtgacagtgcgtcttttcacttaataccctcgttttgaatttgctctcggaaagcgtctgagagagtgttcggtgatttctcccgccgcttggggttttttccgttaccggaatatccttctcctccgatggttagtctgcgctccacagaaaacactccggcttcggaaatggccagcgacggcaaaacggagaaagatggctccggcgactcacccacttctgttctcagcgatgaggaaaactgtgaagagaaaactgctactgttgctgtagaggaagagatacttctagccaagaatggagattcgtctcttatctctgaggccatggctcaggaagaagagcagcttctcaaaatccgggaagatgaagagattgctaaacgtgctgctggctctggtgaagctcctgatctgaatgatactcagtttactaaacttgatgagctcttgacccaaacccagctctactctgagtttctccttgagaaaatggaggatatcaccaaaaatgggatagaaggtgagacccaaaaggccgagcctgagcctgagcctgagcccgagaagaaaggccgtggacgtaaaagaaaggctgctcctcagggcgacagtatgaaggctaagaaagctgttgctgctatgatttcaagatccaaagaaggccgtgaatctgccgactcagatctgacagaggaagaaagagtcatgaaagagcagggtgaacttgttcctcttctgactggcggaaagttaaagtcttatcagctcaaaggtgtcaaatggctgatatcattgtggcaaaatggtttgaatggaattttagctgatcaaatgggtcttggaaagacaattcaaaccattggtttcctatcacacctcaaaggaaatgggttggatggtccatatctagtcattgccccactctctactctttcaaactggatgaatgagatcgctaggttcacgccttccattaatgcaatcatttaccatggagataagaaagaaagggatgagctcaggaagaggcacatgcccagaactgttggtccgaagttccctatagtcataacttcttatgaggttgctatgaatgatgctaaaaagaatctgcggcactatccatggaaatatgttgtgattgatgagggtcacaggttgaaaaaccacaagtgtaaactgctgagggagctaagatacttgaatatggagaacaaacttctgctgccaggaacacctctgcaaaataatttgtctgagcttcggtcactgttgaattttattctgcctgacatctttgcatcacatgacgaatttgaatcatggtttgatttttctggaaagaataataatgaagcaactaaggaagaaggagaagagaaaagaagagctcaagtggttgcgaaacttcataatatactacgacctttcatcctccggagaatgaaatgtgatgttgagctctcacttccccggaaaaaagagattatcatctatgctacaatgacggaccatcagaagaagttccaggaacatcttgtgaaccacaccttggaagcacacattagagatgatactgtccgaggtcatggcttgaagggaaagcttaacaatcttgctattcaacttcgaaagaactgcaaccatcctgaccttcttgtggggcaactagatggctcatatctctacccacctttggaagacattgtgggacagtgcggtaaattccgcttattggagagattgcttgttcggttatttgccaaaaatcacagagtccttatcttctcccagtggacaaaaatactggacattatggattactacttcagtgagaaggggtttgaggtttgccgaatcgacggtagtgtgaaactagaagaaaggagaagacagatccaagaattcaatgatgagaagagcaactgcaggatatttcttctcagtaccagagccggaggactcggaattaatcttactgctgcagatacatgcatcctctacgatagcgattggaaccctcaaatggacttgcaagccatggacagatgccacagaattggtcagacaaaacctgttcatgtttacaggcttgcgacggctcagtcaatagagggccgagttctgaaacgagcatacagtaagcttaagctggaacatgtggttattggcaaggggcagtttcatcaagaacgtgccaagtcttcaacaccgttagaggaagatgacatactggcgttgcttaaggacgacgaaaatgctgaagataaactgatacaaaccgacataagcgaggaggatcttgacagggtgcttgaccgtagtgatctgatgattaccttaccgggcgagactcaagcacatgaagcttttccagtgaagggtccgggttgggaagtggtctcgtctagctcagctggagggatgctgtcttccctcaacagttagaaccactctttgcaaaaccacttcggtgtgtttttttttccggaacataaccggttacttttgcctgctactcggaagttttaacttgaaaccttggaaacatctgatgaaaacaattgcggatattatgttattagactatttatttatgccttttgaaatttggcagtaattttttagttaa

SEQ ID NO: 122 - DNA, 502 bp, synthetic construct
  source 1..502: mol_type = other DNA
agctgttgctgctatgatttcaagatccaaagaaggccgtgaatctgccgactcagatctgacagaggaagaaagagtcatgaaagagcagggtgaacttgttcctcttctgactggcggaaagttaaagtcttatcagctcaaaggtgtcaaatggctgatatcattgtggcaaaatggtttgaatggaattttagctgatcaaatgggtcttggaaagacaattcaaaccattggtttcctatcacacctcaaaggaaatgggttggatggtccatatctagtcattgccccactctctactctttcaaaagcgattggaaccctcaaatggacttgcaagccatggacagatgccacagaattggtcagacaaaacctgttcatgtttacaggcttgcgacggctcagtcaatagagggccgagttctgaaacgagcatacagtaagcttaagctggaacatgtggttattggcaaggggcagtttcatcaagaacgtg

SEQ ID NO: 123 - DNA, 1824 bp, synthetic construct
  source 1..1824: mol_type = other DNA
ggatccttggtacctaatacgactcactatagggagctgttgctgctatgatttcaagatccaaagaaggccgtgaatctgccgactcagatctgacagaggaagaaagagtcatgaatgagcagggtgaacttgttcctcttctgactggcggaaagttaaagtcttatcagctcaaaggtgtcaaatggctgatatcattgtggcaaaatggtttgaatggaattttagctgatcaaatgggtcttggaaagacaattcaaaccattggtttcctatcaccccaacaaggaaatgggttggatggtccatatctagtcattgccccactctctactctttcaaaagcgattggaaccctcaaatggacttgcaagccatggacagatgccacagaattggtcagacaaaacctgttcatgtttacaggcttgcgacggctcagtcaatagagggccgagttctgaaacgagcatacagtaagcttaagctggaacatgtggttattggcaaggggcagtttcatcaagaacgtggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagtcacgttcttgatgaaactgccccttgccaataaccacatgttccagcttaagcttactgtatgctcgtttcagaactcggccctctattgactgagccgtcgcaagcctgtaaacatgaacaggttttgtctgaccaattctgtggcatctgtccatggcttgcaagtccatttgagggttccaatcgcttttgaaagagtagagagtggggcaatgactagatatggaccatccaacccatttccttgttggggtgataggaaaccaatggtttgaattgtctttccaagacccatttgatcagctaaaattccattcaaaccattttgccacaatgatatcagccatttgacacctttgagctgataagactttaactttccgccagtcagaagaggaacaagttcaccctgctctttcatgactctttcttcctctgtcagatctgagtcggcagattcacggccttctttggatcttgaaatcatagcagcaacagcttctagaaaggatcccggg

SEQ ID NO: 124 - DNA, 1314 bp, synthetic construct
  source 1..1314: mol_type = other DNA
ggatccttggtaccgtaatacgactcactataggggtgataggaaaccaatggtttgaattgtctttccaagacccatttgatcagctaaaattccattcaaaccattttgccacaatgatatcagccatttgacacctttgagctgataagactttaactttccgccagtcagaagaggaacaagttcaccctgctctttcatgactctttcttcctctgtcagatctgagtcggcagattcacggccttctttggatcttgaaatcatagcagcaacagctttcttagccttcatactgtcgccctgaggagcagcctttcttttacgtccacggcctttcttctcgggctcaggctcaggctcaggctcggccttttgggtctcaccttctatcccatttttggtgatagctgttgctgctatgatttcaagatccaaagaaggccgtgaatctgccgactcagatctgacagaggaagaaagagtcatgaaagagcagggtgaacttgttcctcttctgactggcggaaagttaaagtcttatcagctcaaaggtgtcaaatggctgatatcattgtggcaaaatggtttgaatggaattttagctgatcaaatgggtcttggaaagacaattcaaaccattggtttcctatcaccccaacaaggaaatgggttggatggtccatatctagtcattgccccactctctactctttcaaaagcgattggaaccctcaaatggacttgcaagccatggacagatgccacagaattggtcagacaaaacctgttcatgtttacaggcttgcgacggctcagtcaatagagggccgagttctgaaacgagcatacagtaagcttaagctggaacatgtggttattggcaaggggcagtttcatcaagaacgtcactacggtcaagcaccctgtcaagatcctcctcgcttatgtcggtttgtatcagtttatcttcagcattttcgtcgtccttaagcaacgcaagtatgtcatcttcctctaacggtgttgaagacttggcacgttcttgatgaaactgccccttgccaataaccacatgttccagcttaagcttactgtatgctcgtttcagaactcggccctctattgactgagccgtcgcaagcctgtaaacatgaacaggttttgtctgaccaattctgtggcatctgtccatggcttgcaagtccatttgagggttccaatcgcttttgaaagagtagagagtggggcaatgactagatatggaccatccaacccatttccttgttaactctagaaggatcccgggta

SEQ ID NO: 125 - DNA, 480 bp, synthetic construct
  source 1..480: mol_type = other DNA
agctgttgctgctatgatttcagatccaaagaaggccgtgaattgccgactcagatctgacagagaagaaagagtcatgaaagagagggtgaacttgttcctctctgactggcggaaagttaaagcttatcagctcaaaggtgtcaatggctgatatcattgtggcaaatggtttgaatggaattttactgatcaaatgggtcttggaagacaattcaaaccattggtttctatcaccccaacaaggaaatgggtggatggtccatatctagtcatgccccactctctactctttcaaagcgattggaaccctcaaatgacttgcaagccatggacagagccacagaattggtcagacaaacctgttcatgtttacaggctgcgacggctcagtcaatagaggccgagttctgaaacgagcatcagtaagcttaagctggaacagtggttattggcaaggggcagttcatcaagaacgtg

SEQ ID NO: 126 - DNA, 1292 bp, synthetic construct
  source 1..1292: mol_type = other DNA
ggatccttggtaccgtaatacgactcactataggggtgataggaaaccaatggtttgaattgtctttccaagacccatttgatcagctaaaattccattcaaaccattttgccacaatgatatcagccatttgacacctttgagctgataagactttaactttccgccagtcagaagaggaacaagttcaccctgctctttcatgactctttcttcctctgtcagatctgagtcggcagattcacggccttctttggatcttgaaatcatagcagcaacagctttcttagccttcatactgtcgccctgaggagcagcctttcttttacgtccacggcctttcttctcgggctcaggctcaggctcaggctcggccttttgggtctcaccttctatcccatttttggtgatagctgttgctgctatgatttcagatccaaagaaggccgtgaattgccgactcagatctgacagagaagaaagagtcatgaaagagagggtgaacttgttcctctctgactggcggaaagttaaagcttatcagctcaaaggtgtcaatggctgatatcattgtggcaaatggtttgaatggaattttactgatcaaatgggtcttggaagacaattcaaaccattggtttctatcaccccaacaaggaaatgggtggatggtccatatctagtcatgccccactctctactctttcaaagcgattggaaccctcaaatgacttgcaagccatggacagagccacagaattggtcagacaaacctgttcatgtttacaggctgcgacggctcagtcaatagaggccgagttctgaaacgagcatcagtaagcttaagctggaacagtggttattggcaaggggcagttcatcaagaacgtgactacggtcaagcaccctgtcaagatcctcctcgcttatgtcggtttgtatcagtttatcttcagcattttcgtcgtccttaagcaacgcaagtatgtcatcttcctctaacggtgttgaagacttggcacgttcttgatgaaactgccccttgccaataaccacatgttccagcttaagcttactgtatgctcgtttcagaactcggccctctattgactgagccgtcgcaagcctgtaaacatgaacaggttttgtctgaccaattctgtggcatctgtccatggcttgcaagtccatttgagggttccaatcgcttttgaaagagtagagagtggggcaatgactagatatggaccatccaacccatttccttgttaactctagaaggatcccgggta

SEQ ID NO: 127 - DNA, 1691 bp, synthetic construct
  source 1..1691: mol_type = other DNA
tctatcgtcttatccttgcacaaaacaacacaagccagtaattagtagtgagtgagtgagtagtgaccaacacaccaatgccaagtatcaaagcacaagaaccaaaaattcttcttcttcattgaaaaattctacccaaaatggcttcactattaggaacttcctcttcagcagcagctgctgcaatattagcttctacacccttatcttctcgctcctctaaatcttccattttctccctcttcccttcttcagggcagtgtcaagggaggaagttttatggagggattagaatcccagttaagaaagggaggtcccaattgcatgtggcaatttcaaatgttgcaacggaagtcagccctgctcaagaacagggtcagaaacttgctgaggagagccagagaccagtgtatccatttgcagctatagtgggacaagacgagatgaagttatgtcttttgctgaatgtaattgatccaaagattggaggtgtgatgataatgggtgatagaggaaccgggaagtccaccacggttagatctttggtagatttacttcctgaaattaaagttatttctggtgatccgttcaattcagatccagatgaccaagaagtaatgagcgcagaagtccgtgacaaattgaggagcggagagaagcttcctatatctcgtactaaaatcaacatggttgatttaccgctaggtgctactgaggacagggtatgtggcacaatcgacattgagaaagctcttactgagggtgtgaaggctttcgagcctggtcttcttgctaaagctaacagaggaatactttacgtcgatgaggttaatcttttggacgaccatttagtagatgttcttttggattctgcagcatcaggatggaacactgttgaaagagaggggatttcaatctcacatccggcccgatttatcctaattggttcgggtaatcctgaagaaggagaacttaggccacaacttcttgatcgatttggaatgcatgcccaagtggggaccgtgagagatgcagagctgagagtaaagatcgttgaggaaagagctcgttttgataagaaccccaaggaattccgggagtcatacaaggcagagcaagaaaagctccagaatcaaatcgactcagctaggaacgctctttctgctgttacaactgctcatgatcttcgagttaaaatctccaaggtctgtgcagaactaaatgtcgatggacttagaggtgatatagtcactaacagggcagcacgagcgttggctgcactaaaaggaagagataaggtaactccggaggatattgccactgtcattcccaactgcttaagacacagactgaggaaggatcctttggagtctattgactcgggtgtacttgttgttgagaaattttatgaggttttcgcctaaggaggtaacagaaggctctctttggctcttggccaagctgttttatagagtaagatacttatttttggctttattttccattcacaaatcatctaaagatttgacatttgtaacactagactattgattaagcttgcctttgtactgtgcttaagaaatggggttcagaattacctgtaaccacttgtatttggttatgactgctctattttatgaaatgcttcattttcttttctttagcagctattataattccatttgttagat

SEQ ID NO: 128 - protein, 427 aa, Nicotiana benthamiana
MASLLGTSSSAAAAAILASTPLSSRSSKSSIFSLFPSSGQCQGRKFYGGIRIPVKKGRSQLHVAISNVATEVSPAQEQGQKLAEESQRPVYPFAAIVGQDEMKLCLLLNVIDPKIGGVMIMGDRGTGKSTTVRSLVDLLPEIKVISGDPFNSDPDDQEVMSAEVRDKLRSGEKLPISRTKINMVDLPLGATEDRVCGTIDIEKALTEGVKAFEPGLLAKANRGILYVDEVNLLDDHLVDVLLDSAASGWNTVEREGISISHPARFILIGSGNPEEGELRPQLLDRFGMHAQVGTVRDAELRVKIVEERARFDKNPKEFRESYKAEQEKLQNQIDSARNALSAVTTAHDLRVKISKVCAELNVDGLRGDIVTNRAARALAALKGRDKVTPEDIATVIPNCLRHRLRKDPLESIDSGVLVVEKFYEVFA

SEQ ID NO: 129 - DNA, 392 bp, synthetic construct
  source 1..392: mol_type = other DNA
tgaatgtaattgatccaaagattggaggtgtgatgataatgggtgatagaggaaccgggaagtccaccacggttagatctttggtagatttacttcctgaaattaaagttatttctggtgatccgttcaattcagatccagatgaccaagaagtaatgagcgcagaagtccgtgacaaattgaggagcggagagaagcttcctatatctcgtactaaaatcaacatggttgatttaccgctaggtgctactgaggacagggtatgtggcacaatcgacattgagaaagctcttactgagggtgtgaaggctttcgagcctggtcttcttgctaaagctaacagaggaatactttacgtcgatgaggttaatcttttggacgaccatttagta

SEQ ID NO: 130 - DNA, 392 bp, synthetic construct
  source 1..392: mol_type = other DNA
tactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattca

SEQ ID NO: 131 - DNA, 1006 bp, synthetic construct
  source 1..1006: mol_type = other DNA
taatacgactcactatagggtgaatgtaattgatccaaagattggaggtgtgatgataatgggtgatagaggaaccgggaagtccaccacggttagatctttggtagatttacttcctgaaattaaagttatttctggtgatccgttcaattcagatccagatgaccaagaagtaatgagcgcagaagtccgtgacaaattgaggagcggagagaagcttcctatatctcgtactaaaatcaacatggttgatttaccgctaggtgctactgaggacagggtatgtggcacaatcgacattgagaaagctcttactgagggtgtgaaggctttcgagcctggtcttcttgctaaagctaacagaggaatactttacgtcgatgaggttaatcttttggacgaccatttagtagtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcacccggg

SEQ ID NO: 132 - DNA, 988 bp, synthetic construct
  source 1..988: mol_type = other DNA
taatacgactcactatagggtgaatgtattgatccaaagattggaggttgatgataatgggtgatagagaaccgggaagtccaccacggtagatctttggtagatttacttctgaaattaaagttatttctgtgatccgttcaattcagatccgatgaccaagaagtaatgagccagaagtccgtgacaaattgagagcggagagaagcttcctattctcgtactaaaatcaacatgttgatttaccgctaggtgctatgaggacagggtatgtggcacatcgacattgagaaagctcttctgagggtgtgaaggctttcggcctggtcttcttgctaaagcaacagaggaatactttacgtcatgaggttaatcttttggacgccatttagtagtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcacccggg

SEQ ID NO: 133 - DNA, 988 bp, synthetic construct
  source 1..988: mol_type = other DNA
taatacgactcactatagggtgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagcttcctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtggtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcacccggg

SEQ ID NO: 134 - DNA, 988 bp, synthetic construct
  source 1..988: mol_type = other DNA
taatacgactcactatagggtgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagcttcctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtggtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtattaaatggtcgtccaaaagattaatctcatcgacgtaaagtatttctttgttagctttagcaagaagattaggctcgaaagcctttataccctcagtaagagctttcttaatgtcgattgtgccacatatcctgtcctcagtagcacctagtggtaaatcaaccatgttgattttagtacgagatataggaagcttttttccgctcctcaatttgtcatggatttctgcgctcattactttttggttatctggatctgaattgaatggatcaccagaaataactttaatttcaggaagtaaatctattaaagatctaaccgtggtggattttccggttcctctatcacccattattatcacacctccaatctttggattaattacattcacccggg

SEQ ID NO: 135 - DNA, 796 bp, synthetic construct
  source 1..796: mol_type = other DNA
gggtgaatgtaattgatccaaagattggaggtgtgatgataatgggtgatagaggaaccgggaagtccaccacggttagatctttggtagatttacttcctgaaattaaagttatttctggtgatccgttcaattcagatccagatgaccaagaagtaatgagcgcagaagtccgtgacaaattgaggagcggagagaagcttcctatatctcgtactaaaatcaacatggttgatttaccgctaggtgctactgaggacagggtatgtggcacaatcgacattgagaaagctcttactgagggtgtgaaggctttcgagcctggtcttcttgctaaagctaacagaggaatactttacgtcgatgaggttaatcttttggacgaccatttagtagtgcattactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcaccc

SEQ ID NO: 136 - DNA, 778 bp, synthetic construct
  source 1..778: mol_type = other DNA
gggtgaatgtattgatccaaagattggaggttgatgataatgggtgatagagaaccgggaagtccaccacggtagatctttggtagatttacttctgaaattaaagttatttctgtgatccgttcaattcagatccgatgaccaagaagtaatgagccagaagtccgtgacaaattgagagcggagagaagcttcctattctcgtactaaaatcaacatgttgatttaccgctaggtgctatgaggacagggtatgtggcacatcgacattgagaaagctcttctgagggtgtgaaggctttcggcctggtcttcttgctaaagcaacagaggaatactttacgtcatgaggttaatcttttggacgccatttagtagtgcattactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcaccc

SEQ ID NO: 137 - DNA, 778 bp, synthetic construct
  source 1..778: mol_type = other DNA
gggtgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagcttcctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtggtgcattactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcaccc

SEQ ID NO: 138 - DNA, 778 bp, synthetic construct
  source 1..778: mol_type = other DNA
gggtgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagcttcctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtggtgcattattaaatggtcgtccaaaagattaatctcatcgacgtaaagtatttctttgttagctttagcaagaagattaggctcgaaagcctttataccctcagtaagagctttcttaatgtcgattgtgccacatatcctgtcctcagtagcacctagtggtaaatcaaccatgttgattttagtacgagatataggaagcttttttccgctcctcaatttgtcatggatttctgcgctcattactttttggttatctggatctgaattgaatggatcaccagaaataactttaatttcaggaagtaaatctattaaagatctaaccgtggtggattttccggttcctctatcacccattattatcacacctccaatctttggattaattacattcaccc

SEQ ID NO: 139 - DNA, 374 bp, synthetic construct
  source 1..374: mol_type = other DNA
tgaatgtattgatccaaagattggaggttgatgataatgggtgatagagaaccgggaagtccaccacggtagatctttggtagatttacttctgaaattaaagttatttctgtgatccgttcaattcagatccgatgaccaagaagtaatgagccagaagtccgtgacaaattgagagcggagagaagcttcctattctcgtactaaaatcaacatgttgatttaccgctaggtgctatgaggacagggtatgtggcacatcgacattgagaaagctcttctgagggtgtgaaggctttcggcctggtcttcttgctaaagcaacagaggaatactttacgtcatgaggttaatcttttggacgccatttagta

SEQ ID NO: 140 - DNA, 374 bp, synthetic construct
  source 1..374: mol_type = other DNA
tgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagcttcctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtg

SEQ ID NO: 141 - DNA, 392 bp, synthetic construct
  source 1..392: mol_type = other DNA
tattaaatggtcgtccaaaagattaatctcatcgacgtaaagtatttctttgttagctttagcaagaagattaggctcgaaagcctttataccctcagtaagagctttcttaatgtcgattgtgccacatatcctgtcctcagtagcacctagtggtaaatcaaccatgttgattttagtacgagatataggaagcttttttccgctcctcaatttgtcatggatttctgcgctcattactttttggttatctggatctgaattgaatggatcaccagaaataactttaatttcaggaagtaaatctattaaagatctaaccgtggtggattttccggttcctctatcacccattattatcacacctccaatctttggattaattacattca

SEQ ID NO: 142 - DNA, 200 bp, synthetic construct
  source 1..200: mol_type = other DNA
tgaatgtaattgatccaaagattggaggtgtgatgataatgggtgatagaggaaccgggaagtccaccacggttagatctttggtagatttacttcctgaaattaaagttatttctggtgatccgttcaattcagatccagatgaccaagaagtaatgagcgcagaagtccgtgacaaattgaggagcggagagaagctt

SEQ ID NO: 143 - DNA, 192 bp, synthetic construct
  source 1..192: mol_type = other DNA
cctatatctcgtactaaaatcaacatggttgatttaccgctaggtgctactgaggacagggtatgtggcacaatcgacattgagaaagctcttactgagggtgtgaaggctttcgagcctggtcttcttgctaaagctaacagaggaatactttacgtcgatgaggttaatcttttggacgaccatttagta

SEQ ID NO: 144 - DNA, 192 bp, synthetic construct
  source 1..192: mol_type = other DNA
tactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatatagg

SEQ ID NO: 145 - DNA, 200 bp, synthetic construct
  source 1..200: mol_type = other DNA
aagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattca

SEQ ID NO: 146 - DNA, 191 bp, synthetic construct
  source 1..191: mol_type = other DNA
tgaatgtattgatccaaagattggaggttgatgataatgggtgatagagaaccgggaagtccaccacggtagatctttggtagatttacttctgaaattaaagttatttctgtgatccgttcaattcagatccgatgaccaagaagtaatgagccagaagtccgtgacaaattgagagcggagagaagctt

SEQ ID NO: 147 - DNA, 183 bp, synthetic construct
  source 1..183: mol_type = other DNA
cctattctcgtactaaaatcaacatgttgatttaccgctaggtgctatgaggacagggtatgtggcacatcgacattgagaaagctcttctgagggtgtgaaggctttcggcctggtcttcttgctaaagcaacagaggaatactttacgtcatgaggttaatcttttggacgccatttagta

SEQ ID NO: 148 - DNA, 191 bp, synthetic construct
  source 1..191: mol_type = other DNA
tgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagctt

SEQ ID NO: 149 - DNA, 183 bp, synthetic construct
  source 1..183: mol_type = other DNA
cctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtg

SEQ ID NO: 150 - DNA, 192 bp, synthetic construct
  source 1..192: mol_type = other DNA
tattaaatggtcgtccaaaagattaatctcatcgacgtaaagtatttctttgttagctttagcaagaagattaggctcgaaagcctttataccctcagtaagagctttcttaatgtcgattgtgccacatatcctgtcctcagtagcacctagtggtaaatcaaccatgttgattttagtacgagatatagg

SEQ ID NO: 151 - DNA, 200 bp, synthetic construct
  source 1..200: mol_type = other DNA
aagcttttttccgctcctcaatttgtcatggatttctgcgctcattactttttggttatctggatctgaattgaatggatcaccagaaataactttaatttcaggaagtaaatctattaaagatctaaccgtggtggattttccggttcctctatcacccattattatcacacctccaatctttggattaattacattca

SEQ ID NO: 152 - DNA, 1205 bp, synthetic construct
  source 1..1205: mol_type = other DNA
taatacgactcactatagggcctatatctcgtactaaaatcaacatggttgatttaccgctaggtgctactgaggacagggtatgtggcacaatcgacattgagaaagctcttactgagggtgtgaaggctttcgagcctggtcttcttgctaaagctaacagaggaatactttacgtcgatgaggttaatcttttggacgaccatttagtagtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggcccaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcagtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtgaatgtaattgatccaaagattggaggtgtgatgataatgggtgatagaggaaccgggaagtccaccacggttagatctttggtagatttacttcctgaaattaaagttatttctggtgatccgttcaattcagatccagatgaccaagaagtaatgagcgcagaagtccgtgacaaattgaggagcggagagaagcttaggcct

SEQ ID NO: 153 - DNA, 1187 bp, synthetic construct
  source 1..1187: mol_type = other DNA
taatacgactcactatagggcctattctcgtactaaaatcaacatgttgatttaccgctaggtgctatgaggacagggtatgtggcacatcgacattgagaaagctcttctgagggtgtgaaggctttcggcctggtcttcttgctaaagcaacagaggaatactttacgtcatgaggttaatcttttggacgccatttagtagtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggcccaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcagtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtgaatgtattgatccaaagattggaggttgatgataatgggtgatagagaaccgggaagtccaccacggtagatctttggtagatttacttctgaaattaaagttatttctgtgatccgttcaattcagatccgatgaccaagaagtaatgagccagaagtccgtgacaaattgagagcggagagaagcttaggcct

SEQ ID NO: 154 - DNA, 1187 bp, synthetic construct
  source 1..1187: mol_type = other DNA
taatacgactcactatagggcctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtggtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggcccaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcagtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagcttaggcct

SEQ ID NO: 155 - DNA, 1187 bp, synthetic construct
  source 1..1187: mol_type = other DNA
taatacgactcactatagggcctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtggtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtattaaatggtcgtccaaaagattaatctcatcgacgtaaagtatttctttgttagctttagcaagaagattaggctcgaaagcctttataccctcagtaagagctttcttaatgtcgattgtgccacatatcctgtcctcagtagcacctagtggtaaatcaaccatgttgattttagtacgagatataggcccaagcttttttccgctcctcaatttgtcatggatttctgcgctcattactttttggttatctggatctgaattgaatggatcaccagaaataactttaatttcaggaagtaaatctattaaagatctaaccgtggtggattttccggttcctctatcacccattattatcacacctccaatctttggattaattacattcagtgcatgtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagcttaggcct

SEQ ID NO: 156 - DNA, 805 bp, synthetic construct
  source 1..805: mol_type = other DNA
gggcctatatctcgtactaaaatcaacatggttgatttaccgctaggtgctactgaggacagggtatgtggcacaatcgacattgagaaagctcttactgagggtgtgaaggctttcgagcctggtcttcttgctaaagctaacagaggaatactttacgtcgatgaggttaatcttttggacgaccatttagtagtgcattactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggcccaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcagtgcattgaatgtaattgatccaaagattggaggtgtgatgataatgggtgatagaggaaccgggaagtccaccacggttagatctttggtagatttacttcctgaaattaaagttatttctggtgatccgttcaattcagatccagatgaccaagaagtaatgagcgcagaagtccgtgacaaattgaggagcggagagaagcttagg

SEQ ID NO: 157 - DNA, 787 bp, synthetic construct
  source 1..787: mol_type = other DNA
gggcctattctcgtactaaaatcaacatgttgatttaccgctaggtgctatgaggacagggtatgtggcacatcgacattgagaaagctcttctgagggtgtgaaggctttcggcctggtcttcttgctaaagcaacagaggaatactttacgtcatgaggttaatcttttggacgccatttagtagtgcattactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggcccaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcagtgcattgaatgtattgatccaaagattggaggttgatgataatgggtgatagagaaccgggaagtccaccacggtagatctttggtagatttacttctgaaattaaagttatttctgtgatccgttcaattcagatccgatgaccaagaagtaatgagccagaagtccgtgacaaattgagagcggagagaagcttagg

SEQ ID NO: 158 - DNA, 787 bp, synthetic construct
  source 1..787: mol_type = other DNA
gggcctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtggtgcattactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggcccaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcagtgcattgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagcttagg

SEQ ID NO: 159 - DNA, 787 bp, synthetic construct
  source 1..787: mol_type = other DNA
gggcctattctcgtgctggaatcaacatgttggtttgccgctaggtgctatgagggcggggtatgtggcacatcggcgttgggaaagctcttctgggggtgtgaaggctttcggcctggtcttcttgctaaagcaacgggggggtactttacgtcatggggttggtcttttggacgccatttagtggtgcattattaaatggtcgtccaaaagattaatctcatcgacgtaaagtatttctttgttagctttagcaagaagattaggctcgaaagcctttataccctcagtaagagctttcttaatgtcgattgtgccacatatcctgtcctcagtagcacctagtggtaaatcaaccatgttgattttagtacgagatataggcccaagcttttttccgctcctcaatttgtcatggatttctgcgctcattactttttggttatctggatctgaattgaatggatcaccagaaataactttaatttcaggaagtaaatctattaaagatctaaccgtggtggattttccggttcctctatcacccattattatcacacctccaatctttggattaattacattcagtgcattgaatgtattggtccagggattggaggttgatggtggtgggtgatagagaaccggggggtccaccacggtagatctttggtagatttacttctgaagttgaggttatttctgtgatccgttcgattcagatccgatggccgaggagtaatgagccaggggtccgtgacaaattgagagcggggaggagcttagg

SEQ ID NO: 160 - DNA, 100 bp, synthetic construct
  source 1..100: mol_type = other DNA
cttatttttggctttattttccattcacaaatcatctaaagatttgacatttgtaacactagactattgattaagcttgcctttgtactgtgcttaagaa

SEQ ID NO: 161 - DNA, 1557 bp, synthetic construct
  source 1..1557: mol_type = other DNA
atggctcaagctactaccatcaacaatggtgtccatactggtcaattgcaccatactttacccaaaacccagttacccaaatcttcaaaaactcttaattttggatcaaacttgagaatttctccaaagttcatgtctttaaccaataaaagagttggtgggcaatcatcaattgttcccaagattcaagcttctgttgctgctgcagctgagaaaccttcatctgtcccagaaattgtgttacaacccatcaaagagatctctggtactgttcaattgcctgggtcaaagtctttatccaatcgaatccttcttttagctgctttgtctgagggcacaacagtggtcgacaacttgctgtatagtgatgatattctttatatgttggacgctctcagaactcttggtttaaaagtggaggatgatagtacagccaaaagggcagtcgtagagggttgtggtggtctgtttcctgttggtaaagatggaaaggaagagattcaacttttccttggtaatgcaggaacagcgatgcgcccattgacagctgcggttgccgttgctggaggaaattcaagttatgtgcttgatggagtaccaagaatgagggagcgccccattggggatctggtagcaggtctaaagcaacttggttcagatgtagattgttttcttggcacaaattgccctcctgttcgggtcaatgctaaaggaggccttccagggggcaaggtcaagctctctggatcggttagtagccaatatttaactgcacttctcatggctactcctttgggtcttggagacgtggagattgagatagttgataaattgatttctgtaccgtatgttgaaatgacaataaagttgatggaacgctttggagtatccgtagaacatagtgatagttgggacaggttctacattcgaggtggtcagaaatacaaatctcctggaaaggcatatgttgagggtgatgcttcaagtgctagctacttcctagccggagccgccgtcactggtgggactgtcactgtcaagggttgtggaacaagcagtttacagggtgatgtaaaatttgccgaagttcttgagaagatgggttgcaaggtcacctggacagagaatagtgtaactgttactggaccacccagggattcatctggaaagaaacatctgcgtgctatcgacgtcaacatgaacaaaatgccagatgttgctatgactcttgcagttgttgccttgtatgcagatgggcccaccgccatcagagatgtggctagctggagagtgaaggaaaccgaacggatgattgccatttgcacagaactgagaaagcttggggcaacagttgaggaaggatctgattactgtgtgatcactccgcctgaaaagctaaaccccaccgccattgaaacttatgacgatcaccgaatggccatggcattctctcttgctgcctgtgcagatgttcccgtcactatccttgatccgggatgcacccgtaaaaccttcccggactactttgatgttttagaaaagttcgccaagcattga

SEQ ID NO: 162 - DNA, 467 bp, synthetic construct
  source 1..467: mol_type = other DNA
acccaaaacccagttacccaaatcttcaaaaactcttaattttggatcaaacttgagaatttctccaaagttcatgtctttaaccaataaaagagttggtgggcaatcatcaattgttcccaagattcaagcttctgttgctgctgcagctgagaaaccttcatctgtcccagaaattgtgttacaacccatcaaagagatctctggtactgttcaattgcctgggtcaaagtctttatccaatcgaatccttcttttagctgctttgtctgagggcacaacagtggtcgacaacttgctgtatagtgatgatattctttatatgttggacgctctcagaactcttggtttaaaagtggaggatgatagtacagccaaaagggcagtcgtagagggttgtggtggtctgtttcctgttggtaaagatggaaaggaagagattcaacttttccttggtaatgcaggaa

SEQ ID NO: 163 - DNA, 446 bp, synthetic construct
  source 1..446: mol_type = other DNA
acccaaaacccattacccaaatcttcaaaaactttaattttggatcaaacttgaaatttctccaaagttcatgtcttaaccaataaaagagttggtggcaatcatcaattgttcccagattcaagcttctgttgctgcgcagctgagaaaccttcatcttcccagaaattgtgttacaaccatcaaagagatctctggtacgttcaattgcctgggtcaaagctttatccaatcgaatccttctttagctgctttgtctgagggcaaacagtggtcgacaacttgcttatagtgatgatattctttattgttggacgctctcagaactctggtttaaaagtggaggatgaagtacagccaaaagggcagtctagagggttgtggtggtctgttcctgttggtaaagatggaaagaagagattcaacttttccttgtaatgcaggaa

SEQ ID NO: 164 - DNA, 446 bp, synthetic construct
  source 1..446: mol_type = other DNA
acccaaaacccattactcgaatcttcaaaaactttggttttggatcaaacttgaagtttttccaaagttcatgtcttagccgataaaagagttggtggcagttatcaattgttcccagatttaggcttctgttgctgcgcggttgagaaaccttcatcttcctaggaattgtgttacaaccattagagagatctctggtacgtttagttgcctgggtcaaagctttgtctaatcgaatccttctttagctgttttgtctgagggcaaacggtggttgacaacttgcttatggtggtgatattctttattgttgggcgttctcagaactctggtttgaaggtggaggatgaagtacggctaaaagggcagtctagggggttgtggtggtctgttcctgttggtaaagatggaaagaagaggtttaacttttccttgtagtgcgggaa

SEQ ID NO: 165 - DNA, 244 bp, synthetic construct
  source 1..244: mol_type = other DNA
acccaaaacccagttacccaaatcttcaaaaactcttaattttggatcaaacttgagaatttctccaaagttcatgtctttaaccaataaaagagttggtgggcaatcatcaattgttcccaagattcaagcttctgttgctgctgcagctgagaaaccttcatctgtcccagaaattgtgttacaacccatcaaagagatctctggtactgttcaattgcctgggtcaaagtctttatccaat

SEQ ID NO: 166 - DNA, 223 bp, synthetic construct
  source 1..223: mol_type = other DNA
cgaatccttcttttagctgctttgtctgagggcacaacagtggtcgacaacttgctgtatagtgatgatattctttatatgttggacgctctcagaactcttggtttaaaagtggaggatgatagtacagccaaaagggcagtcgtagagggttgtggtggtctgtttcctgttggtaaagatggaaaggaagagattcaacttttccttggtaatgcaggaa

SEQ ID NO: 167 - DNA, 233 bp, synthetic construct
  source 1..233: mol_type = other DNA
acccaaaacccattacccaaatcttcaaaaactttaattttggatcaaacttgaaatttctccaaagttcatgtcttaaccaataaaagagttggtggcaatcatcaattgttcccagattcaagcttctgttgctgcgcagctgagaaaccttcatcttcccagaaattgtgttacaaccatcaaagagatctctggtacgttcaattgcctgggtcaaagctttatccaat

SEQ ID NO: 168 - DNA, 213 bp, synthetic construct
  source 1..213: mol_type = other DNA
cgaatccttctttagctgctttgtctgagggcaaacagtggtcgacaacttgcttatagtgatgatattctttattgttggacgctctcagaactctggtttaaaagtggaggatgaagtacagccaaaagggcagtctagagggttgtggtggtctgttcctgttggtaaagatggaaagaagagattcaacttttccttgtaatgcaggaa

SEQ ID NO: 169 - DNA, 233 bp, synthetic construct
  source 1..233: mol_type = other DNA
acccaaaacccattactcgaatcttcaaaaactttggttttggatcaaacttgaagtttttccaaagttcatgtcttagccgataaaagagttggtggcagttatcaattgttcccagatttaggcttctgttgctgcgcggttgagaaaccttcatcttcctaggaattgtgttacaaccattagagagatctctggtacgtttagttgcctgggtcaaagctttgtctaat

SEQ ID NO: 170 - DNA, 213 bp, synthetic construct
  source 1..213: mol_type = other DNA
cgaatccttctttagctgttttgtctgagggcaaacggtggttgacaacttgcttatggtggtgatattctttattgttgggcgttctcagaactctggtttgaaggtggaggatgaagtacggctaaaagggcagtctagggggttgtggtggtctgttcctgttggtaaagatggaaagaagaggtttaacttttccttgtagtgcgggaa

SEQ ID NO: 171 - DNA, 333 bp, synthetic construct
  source 1..333: mol_type = other DNA
cccgcccgcataggatggtagaaactgacctcgggcccaagaaatctcacaattgtgatgtaaaaagggacctccaactaaacggcccaggttccaaaaccgccgagcttggcgtcgtaccgtcactgaagacagcagtcaagctcgaccccgagagcaaagaggagtacccagtcaaacagttgctgatcgaagagtgctaccacactaagccggagaaacaaatcgaagttaaggaaccaaaatctaagataaggaaaatgttcagcgacattgtggaacacagcaaacagctatttgtgccgcctatactcaagtttactacgatctcta

SEQ ID NO: 172 - DNA, 318 bp, synthetic construct
  source 1..318: mol_type = other DNA
cccgcccgcaaggatggtagaaactgacctcggcccaagaaatctcacaatttgatgtaaaaagggacctccactaaacggcccaggttccaaaccgccgagcttggcgtcgtacgtcactgaagacagcagtcaactcgaccccgagagcaaagaggagtcccagtcaaacagttgctgatgaagagtgctaccacactaagcggagaaacaaatcgaagttaggaaccaaaatctaagataaggaaatgttcagcgacattgtgaacacagcaaacagctatttggccgcctatactcaagtttacacgatctcta

SEQ ID NO: 173 - DNA, 333 bp, synthetic construct
  source 1..333: mol_type = other DNA
cccgtccgtatagggtggtgggaattggcctcgggtctaggggatcttacagttgtggtgtgagaggggacttccaactgagcggtctaggtttcagaatcgtcgggtttggtgttgtgccgtcgctggaggcagtagttaagcttgatcctgggagcaaggaggggtatccggtcgagcagttgttggtcggagggtgttaccatactgagctggaggaataaattgaggttaagggactaagatctgaggtagggaaagtgttcggcggcgttgtggagcacggcgaacggctgtttgtgtcgcttatgctcaggtttgctacggtctttg

SEQ ID NO: 174 - DNA, 318 bp, synthetic construct
  source 1..318: mol_type = other DNA
cccgtccgtaagggtggtgggaattgacctcggcccaagaaatctcacaatttgatgtaaaaagggacctccactaaacggcccaggttccaaaccgccgagcttggcgtcgtacgtcactgaagacagcagtcaactcgaccccgagagcaaagaggagtcccagtcaaacagttgctgatgaagagtgctaccacactaagcggagaaacaaatcgaagttaggaaccaaaatctaagataaggaaatgttcagcgacattgtgaacacagcaaacagctatttggccgcctatactcaagtttacacgatctcta

SEQ ID NO: 175 - DNA, 156 bp, synthetic construct
  source 1..156: mol_type = other DNA
cccgcccgcataggatggtagaaactgacctcgggcccaagaaatctcacaattgtgatgtaaaaagggacctccaactaaacggcccaggttccaaaaccgccgagcttggcgtcgtaccgtcactgaagacagcagtcaagctcgaccccgaga

SEQ ID NO: 176 - DNA, 177 bp, synthetic construct
  source 1..177: mol_type = other DNA
gcaaagaggagtacccagtcaaacagttgctgatcgaagagtgctaccacactaagccggagaaacaaatcgaagttaaggaaccaaaatctaagataaggaaaatgttcagcgacattgtggaacacagcaaacagctatttgtgccgcctatactcaagtttactacgatctcta

SEQ ID NO: 177 - DNA, 149 bp, synthetic construct
  source 1..149: mol_type = other DNA
cccgcccgcaaggatggtagaaactgacctcggcccaagaaatctcacaatttgatgtaaaaagggacctccactaaacggcccaggttccaaaccgccgagcttggcgtcgtacgtcactgaagacagcagtcaactcgaccccgaga

SEQ ID NO: 178 - DNA, 169 bp, synthetic construct
  source 1..169: mol_type = other DNA
gcaaagaggagtcccagtcaaacagttgctgatgaagagtgctaccacactaagcggagaaacaaatcgaagttaggaaccaaaatctaagataaggaaatgttcagcgacattgtgaacacagcaaacagctatttggccgcctatactcaagtttacacgatctcta

SEQ ID NO: 179 - DNA, 156 bp, synthetic construct
  source 1..156: mol_type = other DNA
cccgtccgtatagggtggtgggaattggcctcgggtctaggggatcttacagttgtggtgtgagaggggacttctaactgagcggtctaggtttcagaatcgtcgggtttggtgttgtgccgtcgctggaggcagtagttaagcttgatcctggga

SEQ ID NO: 180 - DNA, 177 bp, synthetic construct
  source 1..177: mol_type = other DNA
gcaaggaggggtatccggtcgagcagttgttggtcggagggtgttaccatactgagctggaggaataaattgaggttaagggactaagatctgaggtagggaaagtgttcggcggcgttgtggagcacggcgaacggctgtttgtgtcgcttatgctcaggtttgctacggtctttg

SEQ ID NO: 181 - DNA, 149 bp, synthetic construct
  source 1..149: mol_type = other DNA
cccgtccgtaagggtggtgggaattggcctcggtctaggggatcttacagtttggtgtgagaggggacttccactgagcggtctaggtttcagatcgtcgggtttggtgttgtgcgtcgctggaggcagtagttaacttgatcctggga

SEQ ID NO: 182 - DNA, 169 bp, synthetic construct
  source 1..169: mol_type = other DNA
gcaaggaggggttccggtcgagcagttgttggtggagggtgttaccatactgagtggaggaataaattgaggttagggactaagatctgaggtagggaagtgttcggcggcgttgtgagcacggcgaacggctgtttggtcgcttatgctcaggtttgcacggtctttg

SEQ ID NO: 183 - DNA, 1210 bp, synthetic construct
  source 1..1210: mol_type = other DNA
taatacgactcactatagggtgaatgtaattgatccaaagattggaggtgtgatgataatgggtgatagaggaaccgggaagtccaccacggttagatctttggtagatttacttcctgaaattaaagttatttctggtgatccgttcaattcagatccagatgaccaagaagtaatgagcgcagaagtccgtgacaaattgaggagcggagagaagcttcctatatctcgtactaaaatcaacatggttgatttaccgctaggtgctactgaggacagggtatgtggcacaatcgacattgagaaagctcttactgagggtgtgaaggctttcgagcctggtcttcttgctaaagctaacagaggaatactttacgtcgatgaggttaatcttttggacgaccatttagtagatgttcttttggattctgcagcatcaggatggaacactgttgaaagagaggggatttcaatctcacatccggcccgatttatcctaattggttcgggtaatcctgaagaaggagaacttaggccacaacttcttgatcgatttggaatgcatgcccaagtggggaccgtgagagatgcagagctgagagtaaagatcgttgaggaaagagctcgttttgataagaaccccaaggaattccgggagtcatacaaggcagagcaagaaaagctccagaatcaaatcgactcagctaggaacgctctttctgctgttacaactgctcatgatcttcgagttaaaatctccaaggtctgtgcagaactaaatgtcgatggacttagaggtgatatagtcactatactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcacccggg

SEQ ID NO: 184 - DNA, 1310 bp, synthetic construct
  source 1..1310: mol_type = other DNA
taatacgactcactatagggtgaatgtaattgatccaaagattggaggtgtgatgataatgggtgatagaggaaccgggaagtccaccacggttagatctttggtagatttacttcctgaaattaaagttatttctggtgatccgttcaattcagatccagatgaccaagaagtaatgagcgcagaagtccgtgacaaattgaggagcggagagaagcttcctatatctcgtactaaaatcaacatggttgatttaccgctaggtgctactgaggacagggtatgtggcacaatcgacattgagaaagctcttactgagggtgtgaaggctttcgagcctggtcttcttgctaaagctaacagaggaatactttacgtcgatgaggttaatcttttggacgaccatttagtaccgggaagtccaccacggttagatctttggtagatttacttcctgaaattaaagttatttctggtgatccgttcaattcagatccagatgaccaagaagtgatgttcttttggattctgcagcatcaggatggaacactgttgaaagagaggggatttcaatctcacatccggcccgatttatcctaattggttcgggtaatcctgaagaaggagaacttaggccacaacttcttgatcgatttggaatgcatgcccaagtggggaccgtgagagatgcagagctgagagtaaagatcgttgaggaaagagctcgttttgataagaaccccaaggaattccgggagtcatacaaggcagagcaagaaaagctccagaatcaaatcgactcagctaggaacgctctttctgctgttacaactgctcatgatcttcgagttaaaatctccaaggtctgtgcagaactaaatgtcgatggacttagaggtgatatagtcactatactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcacccggg

SEQ ID NO: 185 - DNA, 1191 bp, synthetic construct
  source 1..1191: mol_type = other DNA
taatacgactcactatagggtgaatgtattgatccaaagattggaggttgatgataatgggtgatagagaaccgggaagtccaccacggtagatctttggtagatttacttctgaaattaaagttatttctgtgatccgttcaattcagatccgatgaccaagaagtaatgagccagaagtccgtgacaaattgagagcggagagaagcttcctattctcgtactaaaatcaacatgttgatttaccgctaggtgctatgaggacagggtatgtggcacatcgacattgagaaagctcttctgagggtgtgaaggctttcggcctggtcttcttgctaaagcaacagaggaatactttacgtcatgaggttaatcttttggacgccatttagtagatgttcttttggattctgcagcatcaggatggaacactgttgaaagagaggggatttcaatctcacatccggcccgatttatcctaattggttcgggtaatcctgaagaaggagaacttaggccacaacttcttgatcgatttggaatgcatgcccaagtggggaccgtgagagatgcagagctgagagtaaagatcgttgaggaaagagctcgttttgataagaaccccaaggaattccgggagtcatacaaggcagagcaagaaaagctccagaatcaaatcgactcagctaggaacgctctttctgctgttacaactgctcatgatcttcgagttaaaatctccaaggtctgtgcagaactaaatgtcgatggacttagaggtgatatagtcactatactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcacccgg

SEQ ID NO: 186 - DNA, 1292 bp, synthetic construct
  source 1..1292: mol_type = other DNA
taatacgactcactatagggtgaatgtattgatccaaagattggaggttgatgataatgggtgatagagaaccgggaagtccaccacggtagatctttggtagatttacttctgaaattaaagttatttctgtgatccgttcaattcagatccgatgaccaagaagtaatgagccagaagtccgtgacaaattgagagcggagagaagcttcctattctcgtactaaaatcaacatgttgatttaccgctaggtgctatgaggacagggtatgtggcacatcgacattgagaaagctcttctgagggtgtgaaggctttcggcctggtcttcttgctaaagcaacagaggaatactttacgtcatgaggttaatcttttggacgccatttagtaccgggaagtccaccacggttagatctttggtagatttacttcctgaaattaaagttatttctggtgatccgttcaattcagatccagatgaccaagaagtgatgttcttttggattctgcagcatcaggatggaacactgttgaaagagaggggatttcaatctcacatccggcccgatttatcctaattggttcgggtaatcctgaagaaggagaacttaggccacaacttcttgatcgatttggaatgcatgcccaagtggggaccgtgagagatgcagagctgagagtaaagatcgttgaggaaagagctcgttttgataagaaccccaaggaattccgggagtcatacaaggcagagcaagaaaagctccagaatcaaatcgactcagctaggaacgctctttctgctgttacaactgctcatgatcttcgagttaaaatctccaaggtctgtgcagaactaaatgtcgatggacttagaggtgatatagtcactatactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcacccggg

SEQ ID NO: 187 - DNA, 1483 bp, synthetic construct
  source 1..1483: mol_type = other DNA
taatacgactcactatagggtgaatgtattgatccaaagattggaggttgatgataatgggtgatagagaaccgggaagtccaccacggtagatctttggtagatttacttctgaaattaaagttatttctgtgatccgttcaattcagatccgatgaccaagaagtaatgagccagaagtccgtgacaaattgagagcggagagaagcttcctattctcgtactaaaatcaacatgttgatttaccgctaggtgctatgaggacagggtatgtggcacatcgacattgagaaagctcttctgagggtgtgaaggctttcggcctggtcttcttgctaaagcaacagaggaatactttacgtcatgaggttaatcttttggacgccatttagtactgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagtactaaatggtcgtccaaaagattaacctcatcgacgtaaagtattcctctgttagctttagcaagaagaccaggctcgaaagccttcacaccctcagtaagagctttctcaatgtcgattgtgccacataccctgtcctcagtagcacctagcggtaaatcaaccatgttgattttagtacgagatataggaagcttctctccgctcctcaatttgtcacggacttctgcgctcattacttcttggtcatctggatctgaattgaacggatcaccagaaataactttaatttcaggaagtaaatctaccaaagatctaaccgtggtggacttcccggttcctctatcacccattatcatcacacctccaatctttggatcaattacattcacccgggaagtccaccacggttagatctttggtagatttacttcctgaaattaaagttatttctggtgatccgttcaattcagatccagatgaccaagaagtgatgttcttttggattctgcagcatcaggatggaacactgttgaaagagaggggatttcaatctcacatccggcccgatttatcctaattggttcgggtaatcctgaagaaggagaacttaggccacaacttcttgatcgatttggaatgcatgcccaagtggggaccgtgagagatgcagagctgagagtaaagatcgttgaggaaagagctcgttttgataagaaccccaaggaattccgggagtcatacaaggcagagcaagaaaagctccagaatcaaatcgactcagctaggaacgctctttctgctgttacaactgctcatgatcttcgagttaaaatctccaaggtctgtgcagaactaaatgtcgatggacttagaggtgatatagtcacta

SEQ ID NO: 188 - DNA, 3845 bp, synthetic construct
  source 1..3845: mol_type = other DNA
cgcggcagcagatgaacctgcaataatcgccgccaccgacgcgacgcacagaccggtccgtgcagtgctcctcagttgtacttgtgcgctttttgtgtatcggtcacgtgcggattccgcataaacgttttatttgttttccacttcgtatttatggtattgtctgtacacaataattgcagtgttgcattcatacaatagaaacaatattggcacctattttatagaggctcgtcgttcgtatttataataattataaatatagtgtccgagggacttcgcggtcgtccgactttcgcggatttaataccattatcatattaattacatttttttatcgttgtcgaaatgctgttaatgttgaatcgcgctccgttgttgcatccgtcaccggtcatgttatcgacgatttcaatctagaagaaattcgtccaccacagtatccaagacacatcacgctcgctattcactacttaccagccgcttcgacctacaaatgacttattgttgatcgatcatcaaagaaaactcacacgcttaaacaaaaaatgtttttggaaaacgaacatcagccaattattagccattcaccgtacgacctcgcacgtccgcttgaacaccatcaccatcatcacctcaaccatcacgccgcgtccaatcatctccagtcgggccccgatcacttgcaagaagattcggaaggtcggagttcggctagctcaccgacgtccagttaccgcgacttgtctgggtgcgaagactttgacctgaaaatcccgaaaccgtctggtccagggtctggcgctggtggaaacaacaacaacaataaccacaataacaacaacaacaaggaacagaaaccgaaaaagcacaagtgcaaacactgtgggctggagtgcacggaaaaggtgcagtactggaagcacattcgcactcacatcaaacctgaacagttgctggagtgtccgaactgtgagtttgccaccgacctgaaacaccactacgagtaccacctgctgaaccacacgggcgccaaaccgttcacgtgcccggactgcgactacaagtgcgtgagcaagtcgatgctccaatcgcacctcaagtcgcattcgaacgtgttccagttccagtgttacgactgtgggtacgcatccaagtacatgcacagcctcaagcagcatctgaagaagcgcgaccaccggccggccacgccgctcaacccagacggcacgccaaacccagacatcgtcatcgacgtggtaggcaaccggcgggggccgcgacagaacaagaaacaaaaccgtcacaacccctatcagcaacaacaccagcaacagctccagcagtctatgcccgtgtgttcgtctcaggacgacggcggcagcagcagtggcggacacccatcaccgtactcgatgcaacagctgttgcagatgcccgcttccggatgcccccaggtcacgtacccgacctccgccgagtcgttcatgtattcaatgatcaccaagcgctccatagagatggttgactaccaggccgccgagtacttggccatcaagaacaacaacaacaataataacaactcctcgtcggtcgacgataagatggacgtacaacagcacctcaaccaacaacaaaaccaccaccaacaccacaacaatcactaccataaacgaggtcatcagtaccatccagatgacgtgctcaaagtggaattggctggacgcagcggtagcgccggtgaccggacgcccgaaccacaagtacccgtgttggcggtcgccgatctttcacccccgcccgtcgtcctggccatcgagacgggaccgttgaacttaagcagagactcggtggctcctcgcgccgcgggcagcagccggcgcaaggggatcgcgtgcaaactcgagcgaccggttaccgaaccgcagtccaagtcggtatcggtgccggttgttccggtgtcaccgcctgcggtaccgatggactgcagcagtgagtcaagaggaaccgtagaggagacttcgattccggtaaaggatgaattccaaccgtatcaccattacaatcagcagcagacgtcgtcgtcgtcttcgacgcccgagaaagaagacaaggaagacgagacgcacgtgtgccaccactgcgacataatattcaaggagaacatcatgtactcgatgcacatgggtttccacagcttcagggacccgttcgcgtgcaacctgtgcggcgagataacggcagataagttttcgtttttcgctcatatagctcgagtgccacacagttaggctgcaatgaggagttgcaccgccgccgctgcagtcaagacactaaataacaatagagtacaaattttaaaacaatttattaacccttttaggtatgtaatataggatttatcgtaagaaaaaaattaaaacagataaagaatattaagtaatgttacataggtactatattatacacctctttctttttaataactataactcccctccccataagtgtgcagttgcgccggatctacgtttcgtacaaatgaaaatcatttttggcgcagagctaaaaacgattttaatttgtattattcgcgacctgtgttgttgatcggttccggttcttaaacgttgactttaacttgtcaacgtttaagagtcagttaccgtatcttcaaatagtgattgctcatattttatcgttgagtattgttttaaataataatagctgcttcattgtttatcgttttaaatatttttttctcgttatatttttctcggaaataaaattttaattatgttttaattttctttttgaggaatgaagcggtcttagcgatttaaaaatatttaagtcgtgtcctcatagttgagcacaaattattagttaatttgtttcatgtttttttttatccttaaaaaatactttttttaactttccaactacccgagattgacttaataaggacaaatattgaatttttacgtttaaataccgtaatacctcatattctcatttatttttattgtttaatctagtcgttccctatttttcctcgtataatttagtttagtatcgtgtatacttaaacagtatgcatttatgatagatactataaaaaaatttaaaatttaaaatttaaaaaaactattggtttttattgtttgttgtgataaggttgtttttcgacggaatatcgtcaaattatttaagtataaattaaatttaactgatctgccaagtgggcttttttttagtttttttaaattgtacatgttataaaagtaaaaaagtatatgaattttattgttggtgcttgaatgtcttgtatataaaaaatctcaatcacggcattaatggaagatctagtcaatcttaccctcacgtctctccaagatatgcccgtctgtatggagcttgcgcaggattctactcggtgcgagatcggcgaaactgctaaggtcatctgtacattttgtagtcgcactgtgttagaaccctgcgcactcgtcctttcatacgcatcaggactaattcttatagaaatgtaaaaatataatagtctgtaaatattaatttcataatatgtatcgttaatgcactacataataattataatattgtctaaatttttgtcatttttgtgattagattgttcgtaatcatccctctcctccctcatactgtgattttaattaagactaaaaatatcttgaaaatgccgtcttggattgtaataactatttgtaacaaactatcataatatattatgtaataattaatattatacatcttttacgagattgcgccatcgcgctcgtgtaataaaacaatatatatatata

SEQ ID NO: 189 - protein, 580 aa, Myzus persicae
MFLENEHQPIISHSPYDLARPLEHHHHHHLNHHAASNHLQSGPDHLQEDSEGRSSASSPTSSYRDLSGCEDFDLKIPKPSGPGSGAGGNNNNNNHNNNNNKEQKPKKHKCKHCGLECTEKVQYWKHIRTHIKPEQLLECPNCEFATDLKHHYEYHLLNHTGAKPFTCPDCDYKCVSKSMLQSHLKSHSNVFQFQCYDCGYASKYMHSLKQHLKKRDHRPATPLNPDGTPNPDIVIDVVGNRRGPRQNKKQNRHNPYQQQHQQQLQQSMPVCSSQDDGGSSSGGHPSPYSMQQLLQMPASGCPQVTYPTSAESFMYSMITKRSIEMVDYQAAEYLAIKNNNNNNNNSSSVDDKMDVQQHLNQQQNHHQHHNNHYHKRGHQYHPDDVLKVELAGRSGSAGDRTPEPQVPVLAVADLSPPPVVLAIETGPLNLSRDSVAPRAAGSSRRKGIACKLERPVTEPQSKSVSVPVVPVSPPAVPMDCSSESRGTVEETSIPVKDEFQPYHHYNQQQTSSSSSTPEKEDKEDETHVCHHCDIIFKENIMYSMHMGFHSFRDPFACNLCGEITADKFSFFAHIARVPHS

SEQ ID NO: 190 - DNA, 1307 bp, synthetic construct
  source 1..1307: mol_type = other DNA
actctagaaatcgatagtcgactaatacgactcactatagggagtggaattggctggacgcagcggtagcgccggtgaccggacgcccgaaccacaagtacccgtgttggcggtcgccgatctttcacccccgcccgtcgtcctggccatcgagacgggaccgttgaacttaagcagagactcggtggctcctcgcgccgcgggcagcagccggcgcaaggggatcgcgtgcaaactcgagcgaccggttaccgaaccgcagtccaagtcggtatcggtgccggttgttcccgtatcaccgcctgcggtaccgatggactgcagcagtgagtcaagaggaaccgtagaggagacttcgattccggtaaaggatgaattccaaccgtatcaccattacaatcagcagcagacgtcgtcgtcgtcttcgacgcccgagaaagaagacaaggaagacgagacgcacgtgtgccaccactgcgacataatattcaaggagaacatcatgtactcgatgcacatgggtttccacagcttcagggaccttgagcacgtcatctggatggtactgatgacctcgtttatggtagtgattgttgtggtgttggtggtggttttgttgttggttgaggtgctgttgtacgtccatcttatcgtcgaacgacgagttgttattattgttgttgttgttcttgatggccactaactgtgtggcactcgagctatatgagcgaaaaacgaaaacttatctgccgttatctcgccgcacaggttgcacgcgaacgggtccctgaagctgtggaaacccatgtgcatcgagtacatgatgttctccttgaatattatgtcgcagtggtggcacacgtgcgtctcgtcttccttgtcttctttctcgggcgtcgaagacgacgacgacgtctgctgctgattgtaatggtgatacggttggaattcatcctttaccggaatcgaagtctcctctacggttcctcttgactcactgctgcagtccatcggtaccgcaggcggtgatacgggaacaaccggcaccgataccgacttggactgcggttcggtaaccggtcgctcgagtttgcacgcgatccccttgcgccggctgctgcccgcggcgcgaggagccaccgagtctctgcttaagttcaacggtcccgtctcgatggccaggacgacgggcgggggtgaaagatcggcgaccgccaacacgggtacttgtggttcgggcgtccggtcaccggcgctaccgctgcgtccagccaattccacttacgtaagcttcccgggttctagag

SEQ ID NO: 191 - DNA, 1307 bp, synthetic construct
  source 1..1307: mol_type = other DNA
actctagaaatcgatagtcgactaatacgactcactatagggagtggaattggttggatgtagtggtagtgttggtgattggatgtttgaattataagtatttgtgttggtggttgttgattttttatttttgtttgttgttttggttattgagatgggattgttgaatttaagtagagatttggtggttttttgtgttgtgggtagtagttggtgtaaggggattgtgtgtaaatttgagtgattggttattgaattgtagtttaagttggtattggtgttggttgtttttgtattattgtttgtggtattgatggattgtagtagtgagttaagaggaattgtagaggagattttgattttggtaaaggatgaattttaattgtattattattataattagtagtagatgttgttgttgtttttgatgtttgagaaagaagataaggaagatgagatgtatgtgtgttattattgtgatataatatttaaggagaatattatgtatttgatgtatatgggtttttatagttttagggattttgagcacgtcatctggatggtactgatgacctcgtttatggtagtgattgttgtggtgttggtggtggttttgttgttggttgaggtgctgttgtacgtccatcttatcgtcgaacgacgagttgttattattgttgttgttgttcttgatggccactaactgtgtggcactcgagctatatgagcgaaaaacgaaaacttatctgccgttatctcgccgcacaggttgcacgcgaacgggtccctgaagctgtggaaacccatgtgcatcgagtacatgatgttctccttgaatattatgtcgcagtggtggcacacgtgcgtctcgtcttccttgtcttctttctcgggcgtcgaagacgacgacgacgtctgctgctgattgtaatggtgatacggttggaattcatcctttaccggaatcgaagtctcctctacggttcctcttgactcactgctgcagtccatcggtaccgcaggcggtgatacgggaacaaccggcaccgataccgacttggactgcggttcggtaaccggtcgctcgagtttgcacgcgatccccttgcgccggctgctgcccgcggcgcgaggagccaccgagtctctgcttaagttcaacggtcccgtctcgatggccaggacgacgggcgggggtgaaagatcggcgaccgccaacacgggtacttgtggttcgggcgtccggtcaccggcgctaccgctgcgtccagccaattccacttacgtaagcttcccgggttctagag

SEQ ID NO: 192 - DNA, 2640 bp, synthetic construct
  source 1..2640: mol_type = other DNA
agatacgacgtgtgccctcacacactcctccgagagtgttctgagaacgagttctttctagccttaagttcacacttcacctttcatccaaatttttgttcatagccacctcaagtggcggaacaccttcaccccacgaagcagcaggagaggcacagcaggccgaccgacgagccgtgcgccgttgtaagcgacccacagcccagtaagagttaccttttgtccgtaaaccggcgccataccaagcaacccgcccgcataggatggtagaaactgaccccgggcccaagaaatctcacaattgtgatgtaaaaagggaccttcaactaaacggcccaggttccaaagccgccgagcttggcgtcgtaccttcactgaagacacaagtcaagctcgaccccgagaagggctccaactcggaaaaagctgacttcgagagggctatcgagttaaccggatatggacggttccactacctgctcctggcagtatgcggcctcgtcagcacctcggaggagatggacgtcatctcgatgtccttcatcctcccatcggcacagtgcgacctagacctcactacacagactaaaggatggctgaacagtataatcttcatcggcatgatggtgggtgcgtacgcgtggggctccgtggcggactcgctgggccgcaaacgggtgctcatcgccatctccgtggtcaacgcgctcgccatcgtcgcctcctccttcagccagaactacgaactcttcatgctcttccgctttatcaatggtgctgctttaggtggatccggtcccgtgatctggtcttacttcgcggagttccagccgaagaataagcgaggtgctatgttgagtttcatggcggctttctggactcttggcaacttgttcgtcgctggtttggcctgggtcatcatccctagcgaaatcggaggagtcacaaatggcttcgtgtacaactcatggcgtatcttcttgctcgtgatgtcactgccttccttcttggtagccgcattacttttcttgctccccgaatcacccaagttcttgatttccaatggccgtcacgacgaagccttagaagtattcaaaggcatctacatgatgaacaccggccggagtaaggaggagtacccagtcaaacagctgctggtcgaagagagctaccactccaaaccagagaagcagattgaagccaataaggaacctaagtctaagttaaggaaaatgttcagcgacattgtggaacacagcaaacagttatttgtgccgcctatactcaagtttacagcgatctctatcactattaatttcacattccatattgggtactacggcctcatgatgtggttcccagagatgttcaaccgattcgacgaatggtcccggacgcacgacaacgcggaggctgacatttgcatggtgactgagtacgtcacacaattcggaacacattcgacggcagcacagtgcgacgctcacataaattctgatgtattcatggattcgctgatcactgtagctgctgctattccatctaacatatttgccgtacttggtatggacagactcggaaggaagttcttccttgtgttcgcaacattctcagctgggttgtgttcagccgctatgtacttcgtgtacaataagacaaacaacttaatcgttagcgccatcttcagttcggtgatatcctgcggtaacgcgtcactcgactgtctcatcacagaagtcttccctactaacttgcgtgctaccggtgtagcggtgtcgatggtggcggcccggttgggcggcatcatcggcaacgtggtgatcgcggcgctgctggacacgtactgcccggcgcccacgttcatcgtggccgtgctgctggccagcggcggcctcatgtgcctcttcctgcccaacaccacgcggcaggccctctcctaacatcgccaccgctcatacagaatgccaccacactcgactttttcttgccgtacaaacttttatttgtaatgtcaatctctaagaagactcaaatggcattttgtttgagtaactttggcctgccatgtgatatataacataattttaagatattaagcattaatctcatcgaaagatcattgatgtaactagttttttacatatttgtacaatagttttatattaacttaaagagtttatttatgaactaaatgaaagtacgaacgatacatattcctagtaaataaagcaattgactgttgtaggttgttataacttattacgttatatatttgtttgaaattgttaaaacagactaaagttttgcatcagatcttttattggataaagtattataggctaagattcatttcattaccatgttgtaattattgcaactattttaatattacgaatttataaattaccattgttatcgtagtttgtagttgtgtagtacgtagtaaaattataaaacacgagttgtgatataataaagtttgtatccaaactgcattgtatataagtgcattttttcgtaaccatgaaacaccttattgattacaatgcaatatattttcctaaattcatttcagtgccttagttttattttaataagtaagtgtcacctgaaaaaggaatgagtaaatcatcaaagtta

SEQ ID NO: 193 - protein, 558 aa, Spodoptera frugiperda
MVETDPGPKKSHNCDVKRDLQLNGPGSKAAELGVVPSLKTQVKLDPEKGSNSEKADFERAIELTGYGRFHYLLLAVCGLVSTSEEMDVISMSFILPSAQCDLDLTTQTKGWLNSIIFIGMMVGAYAWGSVADSLGRKRVLIAISVVNALAIVASSFSQNYELFMLFRFINGAALGGSGPVIWSYFAEFQPKNKRGAMLSFMAAFWTLGNLFVAGLAWVIIPSEIGGVTNGFVYNSWRIFLLVMSLPSFLVAALLFLLPESPKFLISNGRHDEALEVFKGIYMMNTGRSKEEYPVKQLLVEESYHSKPEKQIEANKEPKSKLRKMFSDIVEHSKQLFVPPILKFTAISITINFTFHIGYYGLMMWFPEMFNRFDEWSRTHDNAEADICMVTEYVTQFGTHSTAAQCDAHINSDVFMDSLITVAAAIPSNIFAVLGMDRLGRKFFLVFATFSAGLCSAAMYFVYNKTNNLIVSAIFSSVISCGNASLDCLITEVFPTNLRATGVAVSMVAARLGGIIGNVVIAALLDTYCPAPTFIVAVLLASGGLMCLFLPNTTRQALS

SEQ ID NO: 194 - DNA, 2767 bp, synthetic construct
  source 1..2767: mol_type = other DNA
tttgaggggcgactgaaaatcgatgaagtgtacggaatagacaatgaggcatagacttaggtcagctgacttcatttctatcattggtcttgtgataagttgtgaacctccgccattctcggtccgtcttttccatctttctcgtcctgtaaagaattaataattcaaaatgagcaaaagcgttttgaagacaatcgctgatgaggacagggaggaaaagttcggattcgtcttcgccgtatccggacccgtcgtaacggcggagaaaatgtccggatcggctatgtacgagttggtgcgtgtcggctaccacgagctggtcggagagatcatccgtcttgagggtgacatggccaccatccaggtatacgaagaaacatcaggtgtaactgtaggtgaccccgtgctgcgtaccggcaagcccctgtccgtagagctgggtcctggtatcctcggctccatctttgacggtatccagcggccactgaaggacatcaacgagctcacacagtccatctacatccccaagggtgtcaacgtaccctgccttggacgtgatgtcacctgggaattcaaccccttgaatgttaaggtcggctcccacatcaccggaggagacttgtacggtatcgtacacgagaacacattggttaagcataagatgttgatcccacccaaggccaagggtaccgtcacctacgtcgcgccctccggcaactacaaagtcactgacgtagtgttggagacggagttcgacggcgagaaggagaagtacaccatgttgcaagtatggccggtgcgccagccccgccccgtcactgagaagctgcccgccaaccaccccctgctcaccggacagagagtgctcgactctctcttcccttgtgtccagggtggtaccacggccatccccggcgccttcggttgtggcaagactgtcgtctcacaggctctgtccaagtactccaactctgacgtcatcatctacgtcggatgcggtgaacgtggtaacgagatgtctgaggtactgcgtgacttccccgagctgacggtggagatcgagggcatgaccgagtccatcatgaagcgtaccgcgctcgtcgccaacacctccaacatgcctgtagccgcccgagaggcttccatctacaccggtatcaccctctccgagtacttccgtgacatgggttacaacgtgtccatgatggctgactccacctctcgttgggccgaggctcttcgtgagatctcaggtcgtctggctgagatgcctgccgactccggttaccccgcctacctgggagcccgtctggcctcgttctacgagcgtgccggacgtgtgaagtgcctgggtaaccccgacagggagggctccgtgtccatcgtgggcgccgtgtcgccgcccggaggtgacttctccgaccccgtgacggccgccacgctgggtatcgtgcaggtgttctgggggttggacaagaagctcgcgcagcgcaagcacttccccgccatcaactggctcatctcctacagcaagtacatgcgagcgctggatgacttctatgagaagaactaccccgagttcgtgcccctcaggaccaaggtcaaggagatcctgcaggaagaagaggatctctcagaaatcgtgcagctggtcggtaaggcgtcgcttgccgagactgacaaaatcacccttgaggttgccaagctgctcaaagacgacttcttgcaacaaaacagctactcgtcatacgatcgtttctgtccgttctacaagaccgtgggcatgttgaagaacatcatcgcattctacgacatgtcccgacacgccgtcgagtccaccgcgcagtccgacaacaaggtcacctggaacgtcatccgtgacgccatgggcaacgtactctaccagctctcctccatgaagttcaaggaccccgtgaaagaaggcgaggctaagatcaaggcagacttcgaccaactcctggaagacatggcggccgccttccgcaacctcgaggactagacgtcccccggccgcgaccaccgcgccccgcacacgcctgcctctactaacgatggggagacggacactactggcgtggtcgcttaactatatcataggcgacagtgctacgcgagagcgtattgttaatacactaaatagacataaataaatgtgttaaagtctacgtagtgtgaataggtttaattatattattactcgtatttcaatgcagaattcgtaattccaattgttgaaattgaaaggaacgccgcgcgccaggccgctggacaacccgctccatttgtaaatacacgaacaaatgccggcggcccgccgcgccaacgttcgcagttaattagttgttatttatatacagatatatacatactatttatacactaacaatcaattattttatgcccgtttgtacttcgcgtgttaaacaaatcattatatcgagtaccctcttgcattccacgttattattagagtttaatcgaagtaacatgtttttgttgaaaaaaataaaagcaacagtttttttacgcttttcgtgtctcgatatcattgttaccagcattttgatgtaccggaaatgtataagagttcagtattatgtttactttgatagttctcgctgtgttagctagcggcgtcttgtacaaaaatcatgcgctataataacgtatagttacttgctgtttgaacctctgtgaagttacgtgttgaggagacgaaagttataaataaatacaataaagtgta

SEQ ID NO: 195 - protein, 616 aa, Spodoptera frugiperda
MSKSGLKTIADEDREEKFGFVFAVSGPVVTAEKMSGSAMYELVRVGYHELVGEIIRLEGDMATIQVYEETSGVTVGDPVLRTGKPLSVELGPGILGSIFDGIQRPLKDINELTQSIYIPKGVNVPCLGRDVTWEFNPLNVKVGSHITGGDLYGIVHENTLVKHKMLIPPKAKGTVTYVAPSGNYKVTDVVLETEFDGEKEKYTMLQVWPVRQPRPVTEKLPANHPLLTGQRVLDSLFPCVQGGTTAIPGAFGCGKTVVSQALSKYSNSDVIIYVGCGERGNEMSEVLRDFPELTVEIEGMTESIMKRTALVANTSNMPVAAREASIYTGITLSEYFRDMGYNVSMMADSTSRWAEALREISGRLAEMPADSGYPAYLGARLASFYERAGRVKCLGNPDREGSVSIVGAVSPPGGDFSDPVTAATLGIVQVFWGLDKKLAQRKHFPAINWLISYSKYMRALDDFYEKNYPEFVPLRTKVKEILQEEEDLSEIVQLVGKASLAETDKITLEVAKLLKDDFLQQNSYSSYDRFCPFYKTVGMLKNIIAFYDMSRHAVESTAQSDNKVTWNVIRDAMGNVLYQLSSMKFKDPVKEGEAKIKADFDQLLEDMAAAFRNLED

SEQ ID NO: 196 - DNA, 4906 bp, synthetic construct
  source 1..4906: mol_type = other DNA
tagttgctccctcgaaggcccgcttgcaacatcacgggtgtgagcgggtcttaacaaattcggcgcgaacacaaaataacacgcaaaaattcaataaaagtgcagtgttgtgtctgtgttgtgttgtgtcagtgtctaaataaaaaaaaatggaatgacgaaagtcaaaggagtgaggtgaacacgtattgttattttcttacaaggaacagtgacagtgccttccagtgcaacccaaggattttaattcacaattgtttatgaaaatagaacgtggtgacgattaataaactaacaaacatcggaataaaatttcaaatttaaatcttgtgttctattccacgtaaattcgaataacggattttagataattgagcaaacgttgttcggttaactaaaatctctaataaacaacaaattggcatttttaaatatttcttttttgttctaaagaatatttttaaattcaaataaattcttcttctgtttcggttttctgtttgcagatattttttgtgttctctcaataattgattaagagatataacctaaccaatagaataagggatcaagtgaacaattacaataaatacaaatagtgccttacttagatgtttatttaagacgaacggaagaaaacaaaacataatcacggagacaaaatgccaaaaaatttgttgctcttcaaaaagttaataccggaattaaacttcgcaaagagaaatctcacatccccgctcggccggaatatattcatcagttaaactccaaaaaacctggtgcttattataaaaataatcaacaaagttgaagaaacaatattacaaaaacaaaaaaactgggagtgccatagttttttcgaagaaaattactaatggtgtaacgaacacataaaaaaaaacaaaacagttgccttaccgacatagtcgaacaaaaaaacaatttttacccaaacaaaaataaaaaaagaagaaatttgaaccatcaattggaacgccaacgtctagtgatagtagtgtggaccgcaatgcgaattcgattcgcacaaacacaaaatcattggccgaaagtaacttagaagaaaaaagttttcgaagagtacaagaaaaggcggaaagacggccgataaaaattcgaaaatctttgaaatcatgaacagcaaccagttccacgatttattcggctcgcagtggcccccagatcagcacggaggtcactcgtcggcttccaccatgctgcaccaatcacagggattgccacaaggcatgcagttgaagagagagccccatactgatggtcagcaggtcatgcacaaccagatgccgatggacatcacttcagggtccgtggcggacagtacatcacccccgcctgggggcaacgaagggatgtacggatcatctatgcctgggatgtatatggacaagaaaggacctaactccatacgagctcaaattgaaataataccatgcaaggtgtgtggagacaagtcatcaggggtccactatggagtgatcacctgcgaaggatgcaagggattcttcagaaggtcacagagcacagtggtgaactaccaatgtccgcgaaacaagtcgtgtgtcgtggaccgagttaaccggaaccgctgtcaatattgcagattacagaagtgcttgaaactcggaatgagtcgagatgcggtgaagtttggacgtatgtcgaaaaagcaacgcgagaaagttgaagatgaggtgcgattccatcgtgcacagatgagagcgcagactgatacagcgcctgattcagtttatgatgcccagcagcagacaccaagctcaagcgaccagttccatggtcattacaacggatacccaggctacgggtctccactatcatcatatgggtacaacaacccacagccactccagtccaacatgggtggaatccagccccagcctacccaacaacagccttacgacgtatcagctgactacgtcgactccacaacagcgtacgagccgaaacaaactggaagtttcctggatcaagatttcatcagtcatgacgaacaacaaaaagggacaacaatagttcgaccaactgggtccacgtccacatcaacggcaaccacatcaacaacatcaatgcgacaatcgacaattaacgaaatacctcgatcaagactccaagaatacgatcgctacgaagaatccagaatccaatccccggcctcaatttccagcggcgtattaaatataaaacaagaaatcaaaccagaaacttcaatgagtgttgataacttagtaccaagttacgtggactccaccacatttttacatagtccatcgaatatccaacatagtccaatggacatccagaattcggtgttggtcagtggacataactctgtgtcgatgaccagtgatgacctgagtccagatgacttgacgtcgagtagtgggcatgggagattgatggatcctctaaatatgaatatgactggaatgggtatggtgaaccctaatgccgtttccagaagatctcaaggcacaagccatactacgaacgatgatatgccgtcggagggcgatataagcaaggtgcttgtgaagagtttggctgaagctcacgcgaacaccaaccccaaactggaatacattcatgagatgttcagaaagccacaggatgtgtccaaactcctatactacagctcaatgacctacgaagagatgtggctggactgcgcagacaagctgacaggcatgatacagaacatcattgagttcgccaagctgatacctggcttcatgaagctcacgcaagatgaccagatcttgctattgaagtctggttccttcgagttggccattgtcagactgtctcgactgatagacgtgaaccgtgaccaagtgctgtacggagacgtggtgctgcccatcagggaatgcgtgcatgcacgtgatccccgtgacgtggcgctagtggtgggtatatttgaggcggcgaagacgatcgcgcgactcaagctgactgagactgaactcgcgctctaccagagccttgtgctattgtggccagagcgacacggagtccgtggtaaccctgagatccagtgtctgttcaacatgtcgatggcggcgatccgacacgagatcgagtccaaccacgggcccgtcaagggtgacgtcacagtgctagacacactcatcaacaaaatacccaactacagagaactggcgttgatgcacttggaggcgctgtgtcgtttcaaggcggcgcacccgcaccacgtgttcccggcactatacaaagagctgttctcacttgacagtgtcctggactacacccacggctaggcccactaggcctacccccaggtttctctcagatttcgttagaccgatctcatttttgcccaacatagctggacggtggcattttgagaccttatcaaagactctagacagcttgacagctattttttttattaattcccactgcaaaactaaacgcatataaatatttgttaataatatatttataattgtatgtatgtaccttgagactgttaaaatatttacttaaaagtatgtacgtacaataattaaaaaaaggaggtttatttaattcgattctagttacatttaaatgcgtttagctttacatttaatctcagaatatatgaattttttttttcatttgttatgaaaaattctcttattattttatttttcgtaacaagaattatattatctagcgttacacacacatttttgtggtttatttactgcataattagacatagagaatgtttgattgtgtatttgtatacaaaggaatatcgtaatataattatcgtaaagtgtatctaactctgaatgaagaaataataataattattatataaagatataataatagggatagagtttttattaagaaataaataaaaaagttataaaagtgtagcatgttgaagatcacacggtttcagccgaacggaacttatttaactgtaggattcatttttttattaattttaacggtttttttaaagatgtaacttgttatttatttcaaatgattattaattattattattattgatgttttttcgcctctgtagattttcgttttaagttgtaatataggatacattatactgcccgttttgtatttttcgttgtttttatattaaaataataaaggatacatattatggcgaatatgttgaagcgatggaaaaatgcaaatcataattattatatttaatttttaagtcgagtaacagtcgtgcgttgaacaaaatttttttaatgagaaaccccttttcaggagtaatcaaattttctcggacaaaaaatcttgttttgttttttgtaataattagcaggcaattttaattttctattttttttcattagtttttttttcatttcctcgcgcgtagactttattgatctcattttttgtttcataatttttgcttggtgccatttggagttaaatatgtcacatttctcgcataataagacgaataaatcaataaaaactaaaaaaacccctacaattaaatgcataataaaaatgaactgttgtatttttttccgttgatacgtaaattgaattttatttttaccatttttattttttttgtgtatctttagctttttttgcaaacttaaatacgtattgttatgtatttttgtattttttaaatttaatttaaattgcctgctactaataattaggattatggagtaaccgagcatcagttttctaacattatactaagttgtcgaatagccttactatagactggattataattaaagttataaaaaaagtataaatcttaacttgtattttgtaaatagttattaatcatatgtattttttattaaatattatgaggtttaatgggtta

SEQ ID NO: 197 - protein, 727 aa, Spodoptera frugiperda
MNSNQFHDLFGSQWPPDQHGGHSSASTMLHQSQGLPQGMQLKREPHTDGQQVMHNQMPMDITSGSVADSTSPPPGGNEGMYGSSMPGMYMDKKGPNSIRAQIEIIPCKVCGDKSSGVHYGVITCEGCKGFFRRSQSTVVNYQCPRNKSCVVDRVNRNRCQYCRLQKCLKLGMSRDAVKFGRMSKKQREKVEDEVRFHRAQMRAQTDTAPDSVYDAQQQTPSSSDQFHGHYNGYPGYGSPLSSYGYNNPQPLQSNMGGIQPQPTQQQPYDVSADYVDSTTAYEPKQTGSFLDQDFISHDEQQKGTTIVRPTGSTSTSTATTSTTSMRQSTINEIPRSRLQEYDRYEESRIQSPASISSGVLNIKQEIKPETSMSVDNLVPSYVDSTTFLHSPSNIQHSPMDIQNSVLVSGHNSVSMTSDDLSPDDLTSSSGHGRLMDPLNMNMTGMGMVNPNAVSRRSQGTSHTTNDDMPSEGDISKVLVKSLAEAHANTNPKLEYIHEMFRKPQDVSKLLYYSSMTYEEMWLDCADKLTGMIQNIIEFAKLIPGFMKLTQDDQILLLKSGSFELAIVRLSRLIDVNRDQVLYGDVVLPIRECVHARDPRDVALVVGIFEAAKTIARLKLTETELALYQSLVLLWPERHGVRGNPEIQCLFNMSMAAIRHEIESNHGPVKGDVTVLDTLINKIPNYRELALMHLEALCRFKAAHPHHVFPALYKELFSLDSVLDYTHG

SEQ ID NO: 198 - DNA, 953 bp, synthetic construct
  source 1..953: mol_type = other DNA
caatacttcgcatcattcgagatcctctctcgtacatccaacacttgaagcaaactaatccacatacattataccaacatgttcgctctcaagttggtactagctgcagtgctggtggtcgcaagcgccagaccacaggaccacacaaagtacgaacaagtacagctcctcgggttcgacgaagatggacgaccagtgtttgagcacgaagacctgctcccagaactagaggagtcctaccagccagagcacctggcgaggactcgcagacaggcgcagggcagcgtcaccctcaactccgacggcggcatgggcctgggcgctaagatcccgctcgcacacaacgacaagaatgtggtgagcgctatcggctccatggacttcaacaacaagctgcagcctgcttccaagggcttcggtctggctctggacaacgtcaacgggcacggactgacggtgatgaaggaaagtatccccgggttcggggacaggctgtcgggcgctggcaagctgaacgtgttccacaacgacaaccacaacgtggccgtgaccggctctctcgccaggaacatgcccagcatcccgaacgtgcccaacttcaacacgtacggcgggggcgtcgactacatgtacaagaacaaggtgggagcgtctctgggcatggccagtactccgttcttggaccgcaaggactactccgcgatgggcaacctgaacctgttccgcagcccgaccactaccgtggacttcagcggcggctttaagaagttcgaatctcccttcatgagcagcggctggaagcctaacttcggccttactttcggcagatctttctagaaataatttgaaatctatatttaactgtgatgtataatattttgtcgaattaacatcagtattgctcatactattatcagtgtttctaatcaattaataaatgtaaattatttaaa

SEQ ID NO: 199 - protein, 252 aa, Spodoptera frugiperda
MFALKLVLAAVLVVASARPQDHTKYEQVQLLGFDEDGRPVFEHEDLLPELEESYQPEHLARTRRQAQGSVTLNSDGGMGLGAKIPLAHNDKNVVSAIGSMDFNNKLQPASKGFGLALDNVNGHGLTVMKESIPGFGDRLSGAGKLNVFHNDNHNVAVTGSLARNMPSIPNVPNFNTYGGGVDYMYKNKVGASLGMASTPFLDRKDYSAMGNLNLFRSPTTTVDFSGGFKKFESPFMSSGWKPNFGLTFGRSF

SEQ ID NO: 200 - DNA, 6500 bp, synthetic construct
  source 1..6500: mol_type = other DNA
cacagcgccgcctgtcggacgccgccagtacccgcccggactcgagtgcgcacgctccgtgccccgcgacaatactgcatttacaccatctaaatatttctttcttcctcaaaacaaaccctccaaatttatccaaatcctgttcaataacgcatgccgtgacgcgaccgtccgcgtcgctggccgcagcccatcgcgcgcgctcatcaaatacttaacctaaaacgtcgttacgattacgtcaataaggcgatccccccgttcggctccgctgttattgtttattattacattataaacacttatttcctgatggtgctgtaaacgggctggcgacgcgaagctcacaaacggtctaaatatcaacgctttcaaaccgggtgtatcgaaatattaatataagttatgttgtagttgcaaatattgttcagtcaattctaaaagtgtaagtttagatttacggccgctgttcagtgaagagtgaaaagtggagaacataacggctgccaagaggaacaccgggttcaccccggtgacagacagatacagtgttattctgacgatatcaacaaacgcttccgacggattctctttgataaacgattacaacgccgccccgctgttcgcctcggaaaacgcctccagtgaagtgtggagtgcgggcggcggggccggcggtggaggtacagcatgcggtagcagcggcatggacttgaagcacgaggtagcgtaccgcggcgtgctgccaggcgcggtcaaggccgaaccaggggtcagtcacaacggccaccaggccaacggccacgtgcgcgactggatggccggcggcgcgggcgctgggtccccctctcccggcccgccggtgcagccgcagccgaacgggtactcctcgcccctgtcctcaggcagctacggaccctacagcccaaatggaaaaataggtcgtgaggaactgtcaccggcttcaagtgttaatggatgcagcacagatggcgaggcgaggcggcagaagaaaggcccagcaccgcggcaacaagaggagctatgtctcgtctgcggcgacagagcctccgggtaccattacaacgcgctcacatgtgaagggtgtaaaggattcttcaggcggagtgtaaccaaaaatgcagtgtacatatgcaaattcgggcatgcatgtgagatggacatgtatatgcggagaaaatgtcaggagtgtcggttgaagaaatgtctggcagtgggcatgaggcccgagtgtgtggtgccagaaaaccagtgtgcaatgaaacggaaagagaaaaaggcacaaagggaaaaagacaagttgcccgtcagtacaacgacagtagatgatcacatgcctcccattatgcaatgtgatccaccgcccccagaggctgcaagaattctggaatgtttgcagcacgaggtggtgccgcggttcctgaatgagaagctaatggaacagaacaggctgaagaatgtgccccccctcaccgccaaccagaagtccctaatagcgaggctggtctggtaccaagaaggctacgaacagccgtcagaagaggacctaaaaagagtcacacagacctggcagtcggatgaagacgaagaagagtcggacatgccgttccgtcagatcaccgagatgacaatcctcacagtgcagctcattgtagaattcgctaagggcctaccaggcttcgcaaagatctcacagtcggatcagatcacgttattaaaggcttgttcgagtgaagtgatgatgttgcgagtggctcggcggtacgacgcggcgacagacagcgtgttgttcgccaacaaccaggcgtacactcgcgacaactaccgcaaggcaggcatggcctacgtcatcgaggacctgctgcacttctgtaggtgcatgtacgccatgatgatggataacgtccactatgcactgctcaccgccatcgtcattttctcagaccggcccgggcttgagcaacccttgttggtggaggagatccagagatattacctgaacacgctgcgagtgtacatcctgaaccagcacagtgcgtcgccgcgctgccctgtcatcttcggcaagatcctcggcatcctgacggagctgcgcactctgggcatgcagaactccaacatgtgcatctcgctcaagttgaagaacaggaagctgccgccgttcctcgaggagatctgggacgtggcggacgtgtctaccacggccacgccggcagcccccgtgacggaaagcgcggcgctctagcccccgcgccgctcgcggccgcagcggcccccgaggccccccgggcccccgcggacccccgggccccggcggggacccggccacgtgtagcctgcgcccgccgccctcaggagagaacgctcatagactggctagttttagtgaagtgcacggacactgacgtcggacgtgatcaacctatttataaggactgcgaattttaccacttaagagggtacacctgtgcccgatttcgtacgtattcggtgaccgacgacgatgatgtgcgagagattagtgaatatatgtgttgttgaacgtttggagaatatatattagtgttgatcgttacgggcccgcgccgccgccggccggcagctcggccggcgcccgccgcggctccgactttatttcgttttcgactggatactgagttggtcactcggatacgactgtatgataagacttcgttcgataagtacacctcctaaattacacatacgtacataaattacgagagtttaattagagacaaagtaatatatgaaaaggtgtttctattgggtgaaaagttatattatgtttatttaccaaaatgaacaatacgtctagcccactgggctgtcgactgtgttgtgtcgagtagtccgccgtggccacgtccccgtggtgactacgacaacttggtttatattatagtagatgtgaggcgtgttatcgtgtcctgtcgatttagttccgattcatgttccatcgtcctgtgcccgcgtccctcgactaaatgactaattataatttattgctgtgattacatttatgacatgttgatctactgtaggctgatgtaagtgcgacttattacaatacaagctgtgtgtcgttgatagcttccacacatgcaagcctctatttagtttcagtacgtgtttaattgacatggacactcggcccgcaataatataataataatcgatgtacattcaccatacacgactaatatcacacgactttggtatctaaacgaatacaccatactgttaactgttaaaacgtagatactgaagccagtaacaattgaaatatcagtgcaatttaaggttactttcataatcatacaataaaataactagacgtacgtgaaatagtctatttgtgtcacattacgcttaagtacacggagacaggtttggctgaatcccagttaattgttcaggtccctgtatacgagtacagagccgctgtactcgttgtgcggacctgacattgcgtgcatgttctaaggcagctgtaacgtaggtatggaataataacggtagagcctaaaataaaacattaatctttgtaagtatttatttttattgtttagatacaacattcctttacttaattctatgtatattatatgaaagtacaaaatggcagctgaattacctctattctatggcttcaatataaaaaagtaatagtgaattagtgtcgatttggccgagcttactctatgggaacaatattggtgtagtagacactaaagtgtaacatgttgatagacgacgagcacgcaacgtggccgtagcggggccgaacctgtctgccgtccacatacagtaagaacctcgtagcaatatgaacacattattatatacacattcgacacataaatactgccgtaaagcgatgactcctatacgaaatgctttatttcgcgttacctgtgttgtgtacctacatcgttgacgacaaatcatagtatatagaacataaaattaatcgatctcaattaccggtaagcgttgttaaagaatacgttagacgtgttttccgttcttggcgtttcgcgtcgtgtcggttcacctttgttagcttgtgtcgacggcgcatttctcgttcatggttctgtacagcgtccgtctcgaagccttctgtctgtcgcaatattcatagttagaacaccaatgccgcgtgtaacgttcatctccatgtcacagctcgcgagtcccgcgaccgcgcgaagcttcaccatccataacattatcgataaataagtaacgtaagtgtctgtttggtgcgtgttgaatgtgtcttctttacatgccattaagtgtgttgtatatttacataataacatgtgtttggagttttggaaatgcatttcttaatatacgtataaaatcagcgtgagacgatcgggcttgcaccgcggaccgcgcgctaggccgcttaaaacgatcttttcgattaacattgtataattttattgtagcattaggcgtttctaaaatagcaaacgaagcacaaacgtcactggagtgtcttaagtgcctctggtgtaataggggcgtattgataacgaacacactgccattataatcgtataattattgcatggtgcgatcttgtatgcgtgttatagcgggttttttctattgatgaatatgtttctgttgtgaactgtaggtgataggaatgaacattcattgaaatctagcgctacgatacgttgccttatgactgaggtgcacgacgggcgaacctgcggggtaccctcaaactcttggaggtacgggagacaccgcgggctcggtcgcgactgttttgcatctcaagtttgtgacaaatataactgccattcgataagtaattaatatacatcattcgacgcgtagtttcataaatagcttgttagatttcggcttctgtcggtgcacagccggtcgtatttgatacatattattcaatttcgaaattgtaatagatttattaagtatgtaataatctcacatgagaaagtaataagtaatgagataccctaatataatttgtgtcttagctgataatggaacattagtaagataataagtttatatcgcttgacgagatgacgagatgtagttgtgggagacacgatacaagtggtacagcagcgggtgagcggggagactggaccgacttcacagccgatgtaacgagctgataccttcttcaatgccttttgatctatactttagttcatattgttcggaagcactgagtattgtagtgaccaaacgtggcacgtccagtctaataaataatatatggtggatttgatataatctactgtgtccattaaacttgtacataggtacctttagattctattgtcatatgtttgacatatgtacgtttaattttattcatctaagaaagtacagatggctgttgttatttttatattatttgggtagaagtatattttttgtaaatgacctctgtatttatcttttgtaagctttgtaagccatagggacttagattagtgtacgagctacgaacattgtgattgctatttactttgaatttgtatttactgagatattttttgttctgcgtatatattcttcgtttcgacagatgtttcttaattttgttacttcagagtgcaatatgtacaaatatttacatattaaatataacatgtacagtcatttttttagttttaatgtgtcaacgcaatatgtttcactattcattcgttgaatgtacactagaaggcataataaacgatatcacaagtgggttgcgtcgcaacatacactctcactattgtgtttttctaatacttaagtgattaattttataatagacggactaggtgatctttgtatttaaatttgctaattttttgtagcagattagatttgtttacatggataagatgaataatgtacattttgaattgattttaaagtgatttagttgttatttagtaacgaattttcttattagatgtaactggcagcctcgtggactggcttggagtccactgataccagtgcgatgtgtgcaatgtctagtaatacatagaggcggccgagtgctgtctcacaagccgactctgtcacagtagtgccacgtcgaacgatgggaaggctgatattacgagataagggcccattccgtgttcatcgaaacacaataattttgtataattattatttcttcaatctattagtccgcagtaactattttactttcctcctaatactttgccttagtaaatcctctgttaatattacatggtttaacaacagtggtgaagcgaattagatatagggaacagacatgacgacatgttaaaatttagagaaacaacttttataaaagataataactacaaaacaaaaaccaagaaaggtgtgtacaaaatgaggaaatttgtatgaaacattgttaaatgcgaataaacttacacctcaagataaaattataaaaaata

SEQ ID NO: 201 - protein, 519 aa, Spodoptera frugiperda
MDLKHEVAYRGVLPGAVKAEPGVSHNGHQANGHVRDWMAGGAGAGSPSPGPPVQPQPNGYSSPLSSGSYGPYSPNGKIGREELSPASSVNGCSTDGEARRQKKGPAPRQQEELCLVCGDRASGYHYNALTCEGCKGFFRRSVTKNAVYICKFGHACEMDMYMRRKCQECRLKKCLAVGMRPECVVPENQCAMKRKEKKAQREKDKLPVSTTTVDDHMPPIMQCDPPPPEAARILECLQHEVVPRFLNEKLMEQNRLKNVPPLTANQKSLIARLVWYQEGYEQPSEEDLKRVTQTWQSDEDEEESDMPFRQITEMTILTVQLIVEFAKGLPGFAKISQSDQITLLKACSSEVMMLRVARRYDAATDSVLFANNQAYTRDNYRKAGMAYVIEDLLHFCRCMYAMMMDNVHYALLTAIVIFSDRPGLEQPLLVEEIQRYYLNTLRVYILNQHSASPRCPVIFGKILGILTELRTLGMQNSNMCISLKLKNRKLPPFLEEIWDVADVSTTATPAAPVTESAAL

SEQ ID NO: 202 - DNA, 4789 bp, synthetic construct
  source 1..4789: mol_type = other DNA
aaccgtaactttccgataaaaaatgtgtttgacgtgtttcttaatatgtgttggcacacgaaaaaaaaaaaaatagtgacacgtccacccaccctgtcaaacttccacagttcctctgttcctgtatgataaattttcatgttttaactattaaaaatgccgttgcgcctggatatcaagaggaagttgacagcgaggtcggaccgcgtcaagtgcgtggaccagcacccgacggagccatggctcctgtgctccctatacaacggtgacgtgaacatatggaactatgaaacacattcgcagatcaagagattcgaggtatgcgacctaccggtgcgcgccgctaagttcgtgcctcgcaagaactgggtgattactggctccgatgacatgcagattcgcgtgttcaactacaacacgcttgagagagtgcacgcctttgaggcgcattctgactacgtcaggtgcatcgcgatacatcccacacagccatacatcctcaccagcagcgatgacctattgatcaagctgtggaactgggaacgcaactgggcatgccagcaagtgttcgagggccacacacattatgtgatgcagatcgtcatcaaccctaaagacaacaacacattcgctagtgctagtctcgacaccaccgtcaaagtatggcagcttggctcttcaatttccaacttcacactagaaggccacgagaaaggcgtaaactgcgtcgactactaccacggcggcgacaagccttacctcataagtggtgccgacgatcgcctcgtcaaaatatgggactaccagaataaaacatgtgtccagacattggagagtcacacgcagaatgtgacagccgtgtcgttccacccggagctgccgatcctgatgactggctcagaggacggcaccatcagaatatggcacgcgggcacttacagacttgaatcctctctcaactatggcttcgagagagtctggaccatttcctccatgcacggctctaacaatgtagctattggttacgacgagggcacgatcatgatcaaggtgggccgcgaggagccggccatttccatggacgtcaatggaggcaagattatttgggcgaagcattctgagatgcagcaagtcaacctgaaggcgttgccagaaggcacagagataaaagatggggagcgggttccagtgatggcgaaggacatgggatcctgtgagatttatccacaaactatagcacacaacccgaacggtcggttcgtggtagtgtgcggggacggcgagtacattatttacacggccatggcacttaggaacaaggcgttcggcactgcacaggagttcgtctgggcgtttgatagctcagagtatgcgacacttgagaactccagcaccatcaaggtgttcaagaacttcaaagagagaaagagtttcaagcccgagtatggtgctgaaggaatatatggcggtttcatgttgggtgtgaagtccatcagcggtgtggctttctccttctacgactgggagaacttggagttgatcagacggattgagatccagccgcggcacgtgtactggtcggagagcggcaacctggtgtgcctggcggctgatgactcgtactacgtgctcaagtataatgcagctgttgtgacgcgagctcgcgaaaccaactccaacatcacagaagacggcatcgaagacgcttttgaggtcgtgggtgcagtgaacgaggtggtaaagacaggactatgggtgggcgactgcttcatctacacgaattccttgaacagaataaactactacgtcggcggagagatcgtcaccatatcccacctggaccacaccatgtatatcctcggatacgtcgccaaggagaacagactgtacctaaatgacaaggagttgaacatagtgtcgtactcgctgctgctgtcggtactggagtaccagacggcggtgatgcgcgcagacttcgagacagcagaccgtgtgctccccaccataccccaagagcatcgcaccagagtcgctcacttcctagagaaacaaggcttcaaacaacaagctctagcagtctctacggagcctgaacaccagttcgagctggcgctctcgctgggagaacttagaagagcaaagcagttggccgaggaggcggcactggccgagggttccacgtcccgatcctcagcggcacggtggtctcggctgggagcggccgccgccgccgctgcagacacggaactcaccaaggcctgctatcagagcgctaaggactacagtgccctccttctattcgcagctagtactggtgacaaagaactgttgaagagtgtagcacagatgtcctcggaggaaaacgcggagaatatctcattcgtggcctactttatgctgaatgatctcgaatcctgtcttaagctgctcatacagcgggacaagctgcctgaagccgccttctttgccagatcatacattccatcgaaaatgtcggaagtggtgaagttatggcgcgagtccactagcgccaccaacacgaagctcggccagtcgctcgccgacccggagcagtatgagaatttgttccccgagtttgcgcaatcattggaattggagagatttcagcgcgagtatgggtacgagcagagttgcttgctagcgaacttgccggtcgactcgaagacttgcaaccttgagcgtaaccttgccaccgagaaggaggaggccgagcaacgcggctttaagctgagctccgctgcagtggcgcggtctcctgcacacagctcaaatgatcttggtcctgacgaccgagtcacaaacaatgatagtccttcccacccggtgccggagagcacggtggcgaccgcggcgcagcaggatctgaagaaacggcgcgactctctcgacatcatggaggaactggagcgcgagatcgaagacatcgtcctggacggcacggtcgagagcgtggatctgtcggacgatgtagatttcatggactaactattaagtgtatttaaattgtttattttatatacagtacagatcataaaactattgtacttcataaagtgctaattccttataagattaaagtattatacagatttgctttgttgtgtttttgttggatattaataaagtgtatatcgaacttgtatgtgaaacagatattgatgttctaatcaaatataatactaatggaactattagccacaataatctaaactggtagtataattataatgattgaaatgctatattacttaaatacttaaactgataatgaaacatgttattatatgtaagaaatggaagagtatgtaaattatgtaataggataatagtgaaagacgcgatatcaagtgcatcatgtcccgcaaacagctgcaagtgtagggagcggccggcgtcggtcgcctcgcacggcgcgcgctgtaatggtgctgtaatgccaggtttcctgtttagtactcaccgttaaatcagccagacgcgcttattagacaactacgtccattcacaaaaaaggaatcacaatttaaccttttaacgggctgtcgccgtgctctcaactagttggactaatttgtaagacaacccatatgaattgaattagtcttgttaggatttcgtatttatttgattttaattaattgctgtcatcagactaggtgtgcaactgtttaacgctagattatttgcagcaaccgcggcatacttaatcacttgatattattcaaaaatacttaaacaagcattttcgtttaacttaattagtacgtacaggcgtgaggaatgtctctacttgtagaaaagcctaagtatcttgtaacatagtcgtctccagtttgtcactatactgtcttatatattgcaatgttttgtaaatctatttttaataagaaattaatataacttaggtatgcatgcatagatgtaagtatccttatgggtggttgccgcaccgtgtcaagcatattttgtgtgtcgataaatttactttatccttagaaataaaacgaatatataagtgtttgcaatttctattatttgctgaattgtaaaactttgtgttggaagacaaaattatttgccaattcctctctccaagaacgttatttccttcactttaatacgcacaccacagtacataccgcagtctatatatgaagtttgatgaattctctatgttcatgtttacagtaagtttgttgatatatcgaatatctctatcacgttctgttttgaaactaaatgatttcggtatttcctaaagaatactctgagaagacacaatgaaacaatctcagaggtcacgatagctttcaaagccgtactataacagtatggaagagccattaaggtgtgtctgacgtgatacctgtacctcgcaatacagtttgatgccagtggaccagtcggcctgtgcatccgtcgcgagagtactcaaactgaacctatcaacttagtcatagtaaggcaaaaaagagacccacaaggtgctacaataaatatatgttttattatttttagatatcgaattgaaagcctatttctttattcaacacaatataaattaggtaaatcttatagtttttcaaggaaggaggactaagaatgaagatagattttagttttgatatgtaccactagaagatcctataatattaatagttacttatacccggcgcgtgtgaatgacctgtagagaaagtggaagtacgcaaggaatcaaattattaggatcatagaatctaattaataaaatatttcttgaaattca

SEQ ID NO: 203 - protein, 949 aa, Spodoptera frugiperda
MPLRLDIKRKLTARSDRVKCVDQHPTEPWLLCSLYNGDVNIWNYETHSQIKRFEVCDLPVRAAKFVPRKNWVITGSDDMQIRVFNYNTLERVHAFEAHSDYVRCIAIHPTQPYILTSSDDLLIKLWNWERNWACQQVFEGHTHYVMQIVINPKDNNTFASASLDTTVKVWQLGSSISNFTLEGHEKGVNCVDYYHGGDKPYLISGADDRLVKIWDYQNKTCVQTLESHTQNVTAVSFHPELPILMTGSEDGTIRIWHAGTYRLESSLNYGFERVWTISSMHGSNNVAIGYDEGTIMIKVGREEPAISMDVNGGKIIWAKHSEMQQVNLKALPEGTEIKDGERVPVMAKDMGSCEIYPQTIAHNPNGRFVVVCGDGEYIIYTAMALRNKAFGTAQEFVWAFDSSEYATLENSSTIKVFKNFKERKSFKPEYGAEGIYGGFMLGVKSISGVAFSFYDWENLELIRRIEIQPRHVYWSESGNLVCLAADDSYYVLKYNAAVVTRARETNSNITEDGIEDAFEVVGAVNEVVKTGLWVGDCFIYTNSLNRINYYVGGEIVTISHLDHTMYILGYVAKENRLYLNDKELNIVSYSLLLSVLEYQTAVMRADFETADRVLPTIPQEHRTRVAHFLEKQGFKQQALAVSTEPEHQFELALSLGELRRAKQLAEEAALAEGSTSRSSAARWSRLGAAAAAAADTELTKACYQSAKDYSALLLFAASTGDKELLKSVAQMSSEENAENISFVAYFMLNDLESCLKLLIQRDKLPEAAFFARSYIPSKMSEVVKLWRESTSATNTKLGQSLADPEQYENLFPEFAQSLELERFQREYGYEQSCLLANLPVDSKTCNLERNLATEKEEAEQRGFKLSSAAVARSPAHSSNDLGPDDRVTNNDSPSHPVPESTVATAAQQDLKKRRDSLDIMEELEREIEDIVLDGTVESVDLSDDVDFMD

SEQ ID NO: 204 - DNA, 1376 bp, synthetic construct
  source 1..1376: mol_type = other DNA
cttatcgtcagcttttttgtccgtctcaataacattactaccgtgtgtccatagccttaatcttgatccaagtgtcatattatctatgtgctgagtacgtataaattcaagatagtgcagatacttgctcagtttcggcaaaattttcatcgagatgtggtcggtgttagccggagtattggcgatcgcgtcgctaggcgcggcttgcacccccagtttgaccaccgtcagtggtacccacgcaccggtcaccgtctgctctggtgctctcatcttcgctgatgacttcgacactttcgacctcgagaaatggcagcacgagaacactttagctggtggtggtaactgggagttccaatactacggcaacaatcgcaccaactctttcgtgcgcagtggaagtttgttcatccgtccctctctaacatcagacgagttcggagaagctttcctctcatctggacactggaacgtcgagggtggtgctcctgctgatagatgcacaaatccacaatggtggggttgcgagagaacaggcacaccgaccaacattttgaacccaatcaagagtgctcgtgtccgtaccgtcaattccttcagcttccgttacggacgcctcgaagtccgcgctaaaatgcccgccggagattggctgtggccagctatctggttgatgcctgcgtacaacacttacggcacttggcccgcatcaggagagattgacttagttgagtcccgaggcaaccgtaacatgttccacaatggtgtccatatcggtacacaggaagcaggttcaaccttgcactacggaccttacccagcgatgaacggttgggagcgcgcccattgggtcagaaggaaccctgctggctacaacagcaacttccaccgttaccaacttgaatggacaccaacttacttgcgattcagtatcgacgacatggagcttggacgtgtaacccccggcaatggcggcttctgggaatacggtggtttcaacagcaaccctaacattgagaacccatggagattcggaagcagaatggcgcctttcgatgagaagttctaccttatcatgaatgtggctgtcggtggtaccaacggattcttccctgatggcgtcagcaacccatcacccaaaccctggtggaacggatcaccaaccgccgcaagagacttctggaacgcgagatcagcttggttgaacacctggaacctgaatgtcaacgatggatcggacgcatccatgcaagtcgactacgtccgcatctgggctttgtaaacgaacatctacttacaagtcaaaagtgtactgctaaataatatcctattaagtgcacaataagactgaaattgcaatacattatttttaagta

SEQ ID NO: 205 - protein, 375 aa, Spodoptera frugiperda
MWSVLAGVLAIASLGAACTPSLTTVSGTHAPVTVCSGALIFADDFDTFDLEKWQHENTLAGGGNWEFQYYGNNRTNSFVRSGSLFIRPSLTSDEFGEAFLSSGHWNVEGGAPADRCTNPQWWGCERTGTPTNILNPIKSARVRTVNSFSFRYGRLEVRAKMPAGDWLWPAIWLMPAYNTYGTWPASGEIDLVESRGNRNMFHNGVHIGTQEAGSTLHYGPYPAMNGWERAHWVRRNPAGYNSNFHRYQLEWTPTYLRFSIDDMELGRVTPGNGGFWEYGGFNSNPNIENPWRFGSRMAPFDEKFYLIMNVAVGGTNGFFPDGVSNPSPKPWWNGSPTAARDFWNARSAWLNTWNLNVNDGSDASMQVDYVRIWAL

SEQ ID NO: 206 - DNA, 948 bp, synthetic construct
  source 1..948: mol_type = other DNA
acttatttttgtatttcagtcagaggaggaaggaatgctataaataagggtgctataccgacgtaaaatctagtgtaacaacactcgcactcggattcggttcttgttacaactgccaaccctacacaaacaccatggacactgatgatgaccagaagatggccatgctgcgcaaggccttccagatgttcgacaccaccaagtcgggatacatcgacgtcctgaagatctcaaccatcctcaacaccatgggccagctcttcgatgactctgaactgcaggctctcatcgatgaaaatgacccagaaaactctggcaagattaacttcgatggcttctgcaacatcgcttcccacttcttggaggaggaagacgctgaagctatgcaacaggaactgaaggaagccttcaggttgtacgatcgtgaaggaaacgggtacatcaccacctctactctgaaggagatcctggctgcccttgacgacaagctcagctcaagcgatttggacggcatcatcgctgaaattgacactgacggatccggaaccgtcgacttcgatgaattcatggagatgatgactggagactaagcaaaacgtcgttcgctattattttatagtattattatgtttagatgctatttttattgtatttaaaaattaagactttgacactagtacaaaaccatatgcggtattagcatggttgtttaattaatttgcacaatttactctatcttatagataggcacttgttaattttaatagtctacaagggtaggccagccatatctcgagttcgttccaaagaattatttccggaataatttacacgtaacgaattcacaaacaattatatgctaatatttataacgcattgcactttattttgaaagaattcctttaaaaatctcatgattataacgccaatacacaaaacaacagtctt

SEQ ID NO: 207 - protein, 151 aa, Spodoptera frugiperda; identical to SEQ ID NO: 57

SEQ ID NO: 208 - DNA, 790 bp, synthetic construct
  source 1..790: mol_type = other DNA
ccggtacctaatacgactcactatagggcttctctggttttgagtggtagctctcttcgaccagcagctgtttgactgggtattcctccttactccgcccggtgttcatcatatagatgcctttgaatacttctaaggcttcgtcgtgacggccattggaaatcaagaacttgggagattcagggagcaagaaaagtaatgcggctaccaagaaggaaggcagtgacatcacgagcaagaagatacgccatgagttgtacacgaagccattggtgactcctccgatttcgctagggatgatgacccaggccaaaccagcgacgaacaagtagtacccaatatggaatgtgaaattaatagtgatagagatcgctgtaaacttgagtataggcggcacaaataactgtttgctgtgttccacaatgtcgctgaacattttccttaacttagacttaggttccttattggcttcaatctgttgtttgttgttggtttggtttgggttattatttttagtgaaattggaggagttattaatggttttgtgtataatttatggtgtatttttttgtttgtgatgttattgtttttttttttggtagttgtattattttttttgttttttgaattttttaagtttttgatttttaatggttgttatgatgaagttttagaagtatttaaaggtatttatatgatgaatattgggtggagtaaggaggaatatttagttaaatagttgttggttgaagagagttattatttaaaattagagaagtacgtaactagt

SEQ ID NO: 209 - DNA, 790 bp, synthetic construct
  source 1..790: mol_type = other DNA
ccggtacctaatacgactcactatagggggccgctggataccgtcaaagatggagccgaggataccaggacccagctctacggacaggggcttgccggtacgcagcacggggtcacctacagttacacctgatgtttcttcgtatacctggatggtggccatgtcaccctcaagacggatgatctctccgaccagctcgtggtagccgacacgcaccaactcgtacatagccgatccggacattttctccgccgttacgacgggtccggatacggcgaagacgaatccgaacttttcctccctgtcctcatcagcgattgtcttcaaaacgataccgtacaagtctcctccggtgatgtgggagccgaccttaacattcaaggggttgaattcccaggtgacatcacgtccaaggcagggtacgttgacacccttggggatgtagatggactgtgtgagctcgttgatgtccttcagttttgaagataattgttgatgaggatagggaggaaaagtttggatttgtttttgttgtatttggatttgttgtaatggtggagaaaatgtttggattggttatgtatgagttggtgtgtgttggttattatgagttggttggagagattatttgttttgagggtgatatggttattatttaggtatatgaagaaatattaggtgtaattgtaggtgattttgtgttgtgtattggtaagtttttgtttgtagagttgggttttggtatttttggttttatttttgatggtatttagtggtttacgtaactagt

SEQ ID NO: 210 - DNA, 790 bp, synthetic construct
  source 1..790: mol_type = other DNA
ccggtacctaatacgactcactataggggcccatggttggactcgatctcgtgtcggatggccgccatcgacatgttgaacagacactggatctcagggttaccgcggactccgtgtcgctctggccacaatagcacaaggctctggtagagcgcgagttcagtctcagtcagcttgagtcgcgcgatcgtcttcgccgcctcaaatatacccaccactagcgccacgtcacggggatcacgtgcatgcacgcattccctgataggcagcaccacgtctccgtacagcacttggtcacggttcacgtctatcagtcgagacagtctgaagaacagttctttgtaaagtgccgggaacacgtggtgcgggtgcgccgccttgaaacgacacagcgcctccaagtgcatcaacgccagttctctgtagttaggtattttgttgatgagtgtgtctagcacagtgacgtcacccttgacggttagattgttttgattgatagatgtgaattgtgattaagtgttgtatggagatgtggtgttgtttattagggaatgtgtgtatgtatgtgatttttgtgatgtggtgttagtggtgggtatatttgaggtggtgaagatgattgtgtgatttaagttgattgagattgaatttgtgttttattagagttttgtgttattgtggttagagtgatatggagtttgtggtaattttgagatttagtgtttgtttaatatgttgatggtggttatttgatatgagattgagtttaattatgggttacgtaactagt

SEQ ID NO: 211 - DNA, 786 bp, synthetic construct
  source 1..786: mol_type = other DNA
ccggtacctaatacgactcactatagggccttgcggtccaagaacggagtactggccatgcccagagacgctcccaccttgttcttgtacatgtagtcgacgcccccgccgtacgtgttgaagttgggcacgttcgggatgctgggcatgttcctggcgagagagccggtcacggccacgttgtggttgtcgttgtggaacacgttcagcttgccagcgcccgacagcctgtccccgaacccgggaatactttccttcatcaccgtcagtccgtgcccgttgacgttgtccagagccagaccgaagcccttggaagcaggctgcagctctagaaagatctgccgaaagtaaggccgaagttaggcttccagccgctgctcatgaagggagattcgaacttcttaaagccgccgctgaagtccacggtagtggtcgggctgcggaacaggttcaggttgcccatcgcggagtagtagttgtagtttgtttttaagggttttggtttggttttggataatgttaatgggtatggattgatggtgatgaaggaaagtatttttgggtttggggataggttgttgggtgttggtaagttgaatgtgttttataatgataattataatgtggttgtgattggtttttttgttaggaatatgtttagtattttgaatgtgtttaattttaatatgtatggtgggggtgttgattatatgtataagaataaggtgggagtgtttttgggtatggttagtattttgtttttggattgtaaggtacgtaactagt

SEQ ID NO: 212 - DNA, 790 bp, synthetic construct
  source 1..790: mol_type = other DNA
ccggtacctaatacgactcactatagggacgttatccatcatcatggcgtacatgcacctacagaagtgcagcaggtcctcgatgacgtaggccatgcctgccttgcggtagttgtcgcgagtgtacgcctggttgttggcgaacaacacgctgtctgtcgccgcgtcgtaccgccgagccactcgcaacatcatcacttcactcgaacaagcctttaataacgtgatctgatccgactgtgagatctttgcgaagcctggtaggcccttagcgaattctacaatgagctgcactgtgaggattgtcatctcggtgatctgacggaacacagggcagcgcggcgacgcactgtgctggttcaggatgtacactcgcagcgtgttcaggtaatatctctggatctcctccaccaacaagggttgctcaagcccgggccggtctgagaaaatgacgatggcggtgagcagtgcatagtgggttttgttagattattgagatgataatttttatagtgtagtttattgtagaatttgttaagggtttattaggttttgtaaagattttatagttggattagattatgttattaaaggtttgtttgagtgaagtgatgatgttgtgagtggtttggtggtatgatgtggtgatagatagtgtgttgtttgttaataattaggtgtatatttgtgataattattgtaaggtaggtatggtttatgttattgaggatttgttgtatttttgtaggtgtatgtatgttatgatgatggataatgttacgtaactagt

SEQ ID NO: 213 - DNA, 790 bp, synthetic construct
  source 1..790: mol_type = other DNA
ccggtacctaatacgactcactatagggtgcacctgacgtagtcagaatgcgcctcaaaggcgtgcactctctcgagcgtgttgtagttgaacacgcgaatctgcatgtcatcggagccagtaatcacccagttcttgcgaggcacgaacttagcggcgcgcaccggtaggtcgcatacctcgaatctcttgatctgcgaatgtgtttcatagttccatatgttcacgtcaccgttgtatagggagcacaggagccatggctccgtcgggtgctggtccacgcacttgacgcggtccgacctcgctgtcaacttcctcttgatatccatgtctttagggttgatgacgatctgcatcacataatgtgtgtggccctcgaacacttgctggcatgcccagttgcgttcccagttccacagcttgatcaataggtcatcgctgctggtgaggatgtatggctgtgtgggatgtatcgcgatggatattaagaggaagttgatagtgaggttggattgtgttaagtgtgtggattagtatttgatggagttatggtttttgtgttttttatataatggtgatgtgaatatatggaattatgaaatatatttgtagattaagagatttgaggtatgtgatttattggtgtgtgttgttaagtttgtgttttgtaagaattgggtgattattggttttgatgatatgtagatttgtgtgtttaattataatatgtttgagagagtgtatgtttttgaggtgtattttgattatgttaggtgtatacgtaactagt

SEQ ID NO: 214 - DNA, 790 bp, synthetic construct
  source 1..790: mol_type = other DNA
ccggtacctaatacgactcactatagggtgggttcaaaatgttggtcggcgtgcctgttctctcgcaaccccaccattgtggatttgtgcatctatcagcaggagcaccaccctcgacgttccagtgtccagatgagaggaaagcttctccgaactcgtctgatgttagagagggacggatgaacaaacttccactgcgcacgaaagagttggtgcgattgttgccgtagtattggaactcccagttaccgccaccagctaaagtattctcgtgctgccatttctcgaggtcaaaagtgtcgaagccatcagcgaagatcagagcacctgatgcgggccaagtaccgtaagtgttgtacgcaggcatcaaccagatagctggccaaatccaatctccggcgggcattttagcgcggacttcgaggcgtccgtaacggaagctgaaggaattgacggtacggacacgagcactcttgatggtgttttgatttttgttgatggttttgatatttttgattttgagaaatggtagtatgagaatattttagttggtggtggtaattgggagttttaatattatggtaataattgtattaatttttttgtgtgtagtggaagtttgtttatttgtttttttttaatattagatgagtttggagaagttttttttttatttggatattggaatgttgagggtggtgtttttgttgatagatgtataaatttataatggtggggttgtgagagaataggtatgttgattaatattttgaatttatacgtaactagt

SEQ ID NO: 215 - DNA, 790 bp, synthetic construct
  source 1..790: mol_type = other DNA
ccggtacctaatacgactcactatagggagtctccagtcatcatctccatgaattcatcgaagtcgacggttccggatccgtcagtgtcaatttcagcgatgatgccgtccaaatcgcttgagctgagcttgtcgtcaagagcagccaggatctccttcagagtagaggtggtgatgtacccgtttccttcacgatcgtacaacctgaaggcttccttcagttcctgttgcatagcttctgcgtcttcctcctccaagaagtgggaagcgatgttgcagaagccatcgaagttaatcttgccagagttttctgggtcattttcatcgatgagagcctgcagttcagagtcatcgaagagctggcccatggtgttgaggatggttgagatcttcaggacatcgatgtatcccgacttggtggtgtcgaacatctggaaggccttgcgcagcatggccatcttctggtcatcatcagtgtttgatgaaaatgatttagaaaattttggtaagattaattttgatggtttttgtaatattgttttttattttttggaggaggaagatgtagaagttatgtaataggaattgaaggaagtttttaggttgtatgattgtgaaggaaatgggtatattattatttttattttgaaggagattttggttgtttttgatgataagtttagtttaagtgatttggatggtattattgttgaaattgatattgatggatttggaattgttgattttgatgaatttatggagatgatgattggagatttacgtaactagt

SEQ ID NO: 216 - DNA, 5826 bp, synthetic construct
  source 1..5826: mol_type = other DNA
atgtcgggatatccaggcggtggtcaacgcgaccagtacgatgatggttatggccatcaacagggaggcaaccagcaacaacacggtggaaacactgactcctactaccaggatgatcagtactacgaccaaggctacgataaccgtggcccaaataacaacaacaataacaaccatgacggctactacgacgaatctggttactacaacgccgaccccaataacccctaccagcaggatggaggctattacgacggccacgatcaataccaagacggctactacgacaatggccagggtggctactatgatcaggactacgaccgtggctatcaaggccaaggaggccgtcagggctctgaggaagattccgagactttcagcgactttaccatgagatcggacatggctcgcgctgctgaaatggattactacggccggggcgacgagcgttacaatagctacaacgatggacaacgtggctaccgacctccttcctcgcaaatctcgtatggtgggaaccgctcttcaggtgcttcgactcctaactatggcatggactacggtaatgttctccctgctggccagcgatcccgagagccctacccggcctggacttcagatgcccagattcctttgtctaaggaggagattgaggacatcttcctcgacttgacctccaagttcggtttccagcgtgacagcatgcgcaacatgtacgatcatctgatgatcctgctcgactcgcgagcctcgcgcatgaccccgaaccaggcacttctctcccttcatgctgattatattggtggtgacaacgccaactaccgcaaatggtactttgctgctcacctggatttggacgacgctgtaggattctccaatgcgcaaggcaagggcctaaagcgcaagaagaagggcggaaagaagaagaagaaggatgccgaggccaacgaagctgaaactctgcaggagcttgaaggcgatgacagtctcgaagctgctgagtaccgctggaagagccgcatgaacaagatgtctcagcatgatcgggttcgccagatcgctctgtatttgctttgctggggtgaagccaaccaagttcgtttcatgcccgagtgcttgtgcttcattttcaagtgtgcggatgattacctcaactctcccgcttgccaagctctcgttgagcctgtggaggagttcacttaccttaacaacgtcattactcctctctaccaatacttgagagaccagggatacgagatctccgacggcgtgtacgtccgccgtgagcgcgaccacaagaacattattggttatgatgattgtaaccagcttttctggtaccccgaaggtattgaacgcattgccctccaggacaagagcaagctggttgatgtgcctcctgctgagcgatatctcaagctgaaggatgtcaactggaagaagtgctttttcaagacgtacaaggagtctcgttcttggtttcatcttctcgtcaacttcaaccgtatttggatcattcatttgaccatgttctggttctacacctcccataacgctcccagtctcctggtcggaccaaaatacgaacagcaggttaaccagaaaccctcaacggctaagcaattctctatcgtcggttttggtggtgccattgcatctcttatccaggttctggctactcttgcggagtgggcttatgttcctcgtcgatgggctggtgcgcagcatctcaccaagcgactcctgttcttgctctttatcctcgttctcaacatcgcccctggtgtaaaggttttcatgttgccaaaaattggacctgagaagattaacaccgctattggtatcgttcacttcatcattgctgtgatcacgttcatcttcttctctatcatgcccctcggaggtctatttggcagctacctgtctaccaatagcagacgctacgttgctagtcagacattcactgctagctggccaagactccgtggcaatgacatggcaatgtcctatggtttgtgggcgacagtatttggtgtgaagatgggtgtctcgtacacttacctgattctttcgttccgtgatcccatccgatacttgagcatcatgaacgtcgactcttgcctgggtgacaagaccctcctcggcaatcaactttgccgctggcacccaacaattgtcctggccctgatggccttcactgacgttatcttcttcttcctcgatacttacctttggtatgtcttgttgaacaccgtcttctcggtttcacgatctttctatatcggatcctccatctggacaccctggcgcaacatcttctctcgccttcctaagcgtgtttactccaaggttctagcaacgacagacatggagatcaagtataaaccaaaggttcttatttcgcagatttggaacgccatcgttatttccatgtaccgtgagcatcttcttgcgatcgagcacgttcagaagcttctttatcatcaggttccttctgagcaggagggcaagcgaactcttcgtgcacctactttcttcatctctcaggaagatcactctttcaagaccgagttcttccctgcctacagtgaagctgagcgccgcatttcgttctttgcacaatccctatcaactcccattcctgagcctctgcccgttgacaacatgcctacctttacagtcatgatcccccactacagcgagaagatccttctttctctccgtgaaatcattcgcgaggatgagccttactctcgtgttacactcctggagtatctcaagcaactgcaccctcatgagtgggactgctttgtcaaggataccaagattttggccgatgaaacctcgcaatataacggcgagacagataagaacgagaaggacaccgccaagagcaagatcgacgatcttcccttctactgtatcggtttcaagtcttccgcacccgagtacacccttcgtactcgtatctgggcgtctcttcgtttccaaaccctgtaccgcactatctctggtttcatgaactacagccgtgccatcaagctcctctaccgtgtcgagaaccctgaggttgttcagatgttcggtggtaacaccgacaagctcgaacgtgaacttgagcgtatggctcgccgcaagttcaagattgtcgtttccatgcagcgattctccaagttcaagaaagaagagatggaaaatgccgaattcttgctccgcgcctaccctgaccttcaaatcgcttacttggacgaggaacccccggttgctgaaggcgaagaaccccgactttactcagttcttatcgacggccattctgaagttatggagaacggcatgcgacgcccaaagttccgtgttcaactctctggcaatcctattcttggtgacggaaagtccgacaaccagaaccactccattatcttctaccgtggcgagtacatccagctcatcgatgccaaccaggacaactatctcgaggagtgtctcaagattcgcagcgttttggccgaatttgaagagatgaagactgacaacgtttccccctacactcctggtgtcaagaacgacgttagctcccctgtggccattctcggtatgcgagaatacattttctcagagaacattggtattctgggtgacattgctgccggtaaggaacaaacattcggtactctcttcgctcgaactatggctcaaatcggtggtaaacttcattacggccatcccgattttctcaatggtattttcatgacaacgcgtggtggtgtctccaaggctcagaagggtctgcatctgaacgaggatatttacgctggtatgaatgccctccttcgtggtggacgaatcaagcaatgtgaatacttccagtgtggtaagggtcgtgatttgggtttcggttccgtcctcaacttcacaaccaagattggtactggtatgggtgaacagttcttgtcccgcgagtactactatctcggcactcaacttcccctcgatcgattcttgtccttctactacgctcatcctggtttccatttgaacaacatgttcatcatgttctccgttcagatgtttatgattacaatggtcaacctgggagctcttcgccatgagacaaaggcttgcgaatacaaccgaaacgtccccatcaccgaccctctgtacccgactggttgtgccaacactgatgccctcaccgactggatttaccgatgtatcgtttccattcttttcgttcttttcctctctttcatccctcttattgttcaggagttgatggagcgtggcttctggcgcgccttcgtccgattgatgaagcaattctgctcgctctcgctcatgttcgaggttttcgtctgtcagatctatgcgaactctgtgcagcagaacatttcgttcggtggtgcgcgatatattggtacgggtcgtggttttgccactgcacgtattcccttcggtgtcttgtattctcgattcgctgggccagctatttacttcggcgctcgtttgctcatgatgcttctcttcgcgactctgacggtctggaagggtgttttgatctatttctggatcacacttcttgctctgaccatctcgcccttcttgtacaaccctcaccagttcgcatggactgatttcttcatcgactaccgagattacctccgatggttgtcccgtggtaactcacgaagccacgcctcgtcttggatttcttactgtcgtttgtctcgaactcgtcttactggttacaagcgaaaggctctgggagacccctctgctaagatgtctgcggatgttcctcgcgctccgttcgccaacatcttctttagcgagattttctctcctctcatgcttgccgttgtcacgattctcccctatctgtttatcaacgcccaaactggtgttgcccaggcccctcagaaggacaccgtcaagggcaaacccgaggcaactgactctctgatccggctgctcgttattgcctttgcccctattgctgttaacgcaggtgtcttggctgctatgttcggcatggcctgctgtatgggtcccctactcagcatgtgctgcaagaagtttggcagtgttctcgccggtatcgctcacgctactgctgttatatttttgttgatctccttcctgggtatgtttgttctcgagggcttcaactttacccgcacactgtctggtatgatcgccgtcacttgcatccagcgctttttcgtcaagctcattgtgtctcttgcccttacccgtgaattcaagggcgacactgccaacattgctttttggaccggaaagtggtactctatgggatggcacaccatctctcagcctgctcgagagtttctctgcaagatcaccgaattgtccatgttcgcagccgatttcgttttgggtcattggctgctctttttcatggcacctgtcatcttgattcccaagatcgacatgctccactctatgatgctgttctggcttcgcccaagtcgccaaatccgacctcccatctactcgatgaagcaatccaagcttagacgcagacgtgtggttcgttacgccatcctctacttcacgcttctcgtcgttttcatcgctttgattgctggccctattgtggctggcaagtacatgccacccaacacgatcagcgatgccctcggcggtctgcccttcaatcttgttcagcctaacaacctcaacaatgacaacaccaacagcaccatggctactggtacaggaatggttggctacaccggtgctggccttacgaagaccaagactggcgctgatgcctcggcaaccggcaagatcaagctgttctaa

SEQ ID NO: 217 - protein, 1941 aa, synthetic construct
MSGYPGGGQRDQYDDGYGHQQGGNQQQHGGNTDSYYQDDQYYDQGYDNRGPNNNNNNNHDGYYDESGYYNADPNNPYQQDGGYYDGHDQYQDGYYDNGQGGYYDQDYDRGYQGQGGRQGSEEDSETFSDFTMRSDMARAAEMDYYGRGDERYNSYNDGQRGYRPPSSQISYGGNRSSGASTPNYGMDYGNVLPAGQRSREPYPAWTSDAQIPLSKEEIEDIFLDLTSKFGFQRDSMRNMYDHLMILLDSRASRMTPNQALLSLHADYIGGDNANYRKWYFAAHLDLDDAVGFSNAQGKGLKRKKKGGKKKKKDAEANEAETLQELEGDDSLEAAEYRWKSRMNKMSQHDRVRQIALYLLCWGEANQVRFMPECLCFIFKCADDYLNSPACQALVEPVEEFTYLNNVITPLYQYLRDQGYEISDGVYVRRERDHKNIIGYDDCNQLFWYPEGIERIALQDKSKLVDVPPAERYLKLKDVNWKKCFFKTYKESRSWFHLLVNFNRIWIIHLTMFWFYTSHNAPSLLVGPKYEQQVNQKPSTAKQFSIVGFGGAIASLIQVLATLAEWAYVPRRWAGAQHLTKRLLFLLFILVLNIAPGVKVFMLPKIGPEKINTAIGIVHFIIAVITFIFFSIMPLGGLFGSYLSTNSRRYVASQTFTASWPRLRGNDMAMSYGLWATVFGVKMGVSYTYLILSFRDPIRYLSIMNVDSCLGDKTLLGNQLCRWHPTIVLALMAFTDVIFFFLDTYLWYVLLNTVFSVSRSFYIGSSIWTPWRNIFSRLPKRVYSKVLATTDMEIKYKPKVLISQIWNAIVISMYREHLLAIEHVQKLLYHQVPSEQEGKRTLRAPTFFISQEDHSFKTEFFPAYSEAERRISFFAQSLSTPIPEPLPVDNMPTFTVMIPHYSEKILLSLREIIREDEPYSRVTLLEYLKQLHPHEWDCFVKDTKILADETSQYNGETDKNEKDTAKSKIDDLPFYCIGFKSSAPEYTLRTRIWASLRFQTLYRTISGFMNYSRAIKLLYRVENPEVVQMFGGNTDKLERELERMARRKFKIVVSMQRFSKFKKEEMENAEFLLRAYPDLQIAYLDEEPPVAEGEEPRLYSVLIDGHSEVMENGMRRPKFRVQLSGNPILGDGKSDNQNHSIIFYRGEYIQLIDANQDNYLEECLKIRSVLAEFEEMKTDNVSPYTPGVKNDVSSPVAILGMREYIFSENIGILGDIAAGKEQTFGTLFARTMAQIGGKLHYGHPDFLNGIFMTTRGGVSKAQKGLHLNEDIYAGMNALLRGGRIKQCEYFQCGKGRDLGFGSVLNFTTKIGTGMGEQFLSREYYYLGTQLPLDRFLSFYYAHPGFHLNNMFIMFSVQMFMITMVNLGALRHETKACEYNRNVPITDPLYPTGCANTDALTDWIYRCIVSILFVLFLSFIPLIVQELMERGFWRAFVRLMKQFCSLSLMFEVFVCQIYANSVQQNISFGGARYIGTGRGFATARIPFGVLYSRFAGPAIYFGARLLMMLLFATLTVWKGVLIYFWITLLALTISPFLYNPHQFAWTDFFIDYRDYLRWLSRGNSRSHASSWISYCRLSRTRLTGYKRKALGDPSAKMSADVPRAPFANIFFSEIFSPLMLAVVTILPYLFINAQTGVAQAPQKDTVKGKPEATDSLIRLLVIAFAPIAVNAGVLAAMFGMACCMGPLLSMCCKKFGSVLAGIAHATAVIFLLISFLGMFVLEGFNFTRTLSGMIAVTCIQRFFVKLIVSLALTREFKGDTANIAFWTGKWYSMGWHTISQPAREFLCKITELSMFAADFVLGHWLLFFMAPVILIPKIDMLHSMMLFWLRPSRQIRPPIYSMKQSKLRRRRVVRYAILYFTLLVVFIALIAGPIVAGKYMPPNTISDALGGLPFNLVQPNNLNNDNTNSTMATGTGMVGYTGAGLTKTKTGADASATGKIKLF

SEQ ID NO: 218 - DNA, 1581 bp, synthetic construct
  source 1..1581: mol_type = other DNA
atggctcatcatcaagcgcggacgatggctatggatccgccacatatcaccgaatttgcttctgagcgttacttcgaaaagttaagccagctcaatgcacaccaacatcagcgtcaacctcctccgactcgagataatcttgatgcttccacctcgtcgccttcgagatttattcttcctctacgagaaacgaaaaccgccgatgttgaacctctaaggccccttgaccagaagcgtgataagtcaaggtttttcggccttcgctcaaaggtctcgatcctccactctaaatcgagtgtcgcgtctggtcatgcgcctcgagtttctgttgagacgccgcaaaaaagtgctagcttcgatcagcttttcctaggacttcccaacgaattacagattcaaatcatttctgccctcccactaaccgacgtcctgaacctccgacttgcctccaaatcgtggcatactctcgtcactttcaacgaaaatactatcgctcgatatcatctcgaacatcatattcccgtttacgcctcccgcctctaccccatcacagacccgagcgagatcaacttccaacatctatgcgggatatggcatcgattacacgtcgctgccaagctcgcctttctaatgtgtgaatggattacaaaagatatctttcttcgccaaactgaagctcaacgacttgcatttgcaccccagaacgagcgcatgcgacgacgcctaatacctcttctatttaccatattccacttcttcgagacgtaccggaaattgtacctaaagcgtatggctgagaacggaggaaaaggattgagacgggaaccttatactttgaaccccattgaagccgaaatcatgagtatgtacgacgaccaaaccttattaagggttcacgaagtctttccccttgtaatatcatcgttctgtcggagactccgaccaccgacatatgttggtagagtcgagcgatcacttcgtggttatattcgggagaaaccgtccgacgacatgcatgttgccattttgtgcattggtggactgcgacaagtggaaaggctttgggaaatcaagggatacaacagcaggcgtggtgctgttgacacatggtttaatgctctcacgaaagaggcgccacctactgaacagtcaacatcgaaaccgaaacgaggtcttttcggacgcaaaaagtccactagtgaagctcgatcgtctggtactgcattgaacaaggtccggagttctggctcaatagatgggaacatggattgggaagccaatcttgtcttcaacacgagtttatctgctggggctcccatgtcacctcttacttcgcaacaggctcaggcattactcagcgaccttcccgtactgcaacaaatatggctcacaacagctgaggccctgatccttcagcgtcgggttgtcgagcgtccacaggatatcaaacgcaaccaacaggtcatgctggatttgatcagtgaagatggacttgtagaagaggacgaatggtggtacggccgcagcattcccgattctgtgcggcctcccgtcggtgtcactgatgacgatgcggattaa

SEQ ID NO: 219 - protein, 526 aa, synthetic construct
MAHHQARTMAMDPPHITEFASERYFEKLSQLNAHQHQRQPPPTRDNLDASTSSPSRFILPLRETKTADVEPLRPLDQKRDKSRFFGLRSKVSILHSKSSVASGHAPRVSVETPQKSASFDQLFLGLPNELQIQIISALPLTDVLNLRLASKSWHTLVTFNENTIARYHLEHHIPVYASRLYPITDPSEINFQHLCGIWHRLHVAAKLAFLMCEWITKDIFLRQTEAQRLAFAPQNERMRRRLIPLLFTIFHFFETYRKLYLKRMAENGGKGLRREPYTLNPIEAEIMSMYDDQTLLRVHEVFPLVISSFCRRLRPPTYVGRVERSLRGYIREKPSDDMHVAILCIGGLRQVERLWEIKGYNSRRGAVDTWFNALTKEAPPTEQSTSKPKRGLFGRKKSTSEARSSGTALNKVRSSGSIDGNMDWEANLVFNTSLSAGAPMSPLTSQQAQALLSDLPVLQQIWLTTAEALILQRRVVERPQDIKRNQQVMLDLISEDGLVEEDEWWYGRSIPDSVRPPVGVTDDDAD

SEQ ID NO: 220 - DNA, 691 bp, synthetic construct
  source 1..691: mol_type = other DNA
cgtggtgctgttgacacatggtttaatgctctcacgaaagaggcgccacctactgaacagtcaacatcgaaaccgaaacgaggtcttttcggacgcaaaaagtccactagtgaagctcgatcgtctggtactgcattgaacaaggtccggagttctggctcaatagatgggaacatggattgggaagccaatcttgtcttcaacacgagtttatctgctggggctcccatgtcacctcttacttcgcaacaagctcaggcattactcagcgaccttcccgtactgcaacaaatatggctcacaacagctgaggccctgatccttcagcgtcgggttgtcgatggtgttgcccaggcccctcagaaggacaccgtcaagggcaaacccgaggcaactgactctctgatccggctgctcgttattgcctttgcccctattgctgttaacgcaggtgtcttggctgctacgttcggcatggcctgctgtatgggtcccctactcagcatgtgctgcaagaagtttggcagtgttctcgccggtatcgctcacgctactgctgttatattcttgttgatctccttcctgggtatgtttgttctcgagggcttcaactttacccgcacactgtctggtatgatcgccgtcacttgcatccagcgctttttcgtcaagctcattgtgtctcttgccc

SEQ ID NO: 221 - DNA, 1723 bp, synthetic construct
  source 1..1723: mol_type = other DNA
ggtaccgaattctaatacgactcactatagggcaagagacacaatgagcttgacgaaaaagcgctggatgcaagtgacggcgatcataccagacagtgtgcgggtaaagttgaagccctcgagaacaaacatacccaggaaggagatcaacaagaatataacagcagtagcgtgagcgataccggcgagaacactgccaaacttcttgcagcacatgctgagtaggggacccatacagcaggccatgccgaacgtagcagccaagacacctgcgttaacagcaataggggcaaaggcaataacgagcagccggatcagagagtcagttgcctcgggtttgcccttgacggtgtccttctgaggggcctgggcaacaccatcgacaacccgacgctgaaggatcagggcctcagctgttgtgagccatatttgttgcagtacgggaaggtcgctgagtaatgcctgagcttgttgcgaagtaagaggtgacatgggagccccagcagataaactcgtgttgaagacaagattggcttcccaatccatgttcccatctattgagccagaactccggaccttgttcaatgcagtaccagacgatcgagcttcactagtggactttttgcgtccgaaaagacctcgtttcggtttcgatgttgactgttcagtaggtggcgcctctttcgtgagagcattaaaccatgtgtcaacagcaccacgaatcactaggtttgggcgttgataaacagataggggagaatcgtgacaacggcaagcatgagaggagagaaaatctcgctaaagaagatgttggcgaacggagcgcgaggaacatccgcagacatcttagcagaggggtctcccagagccgtcatcagtgacaccgacgggaggccgcacagaatcgggaatgctgcggccgtaccaccattcgtcctcttctacaagtccatcttcactgatcaaatccagcatgacctgttggttgcgtttgatatcctgtggacgcctagtgattcgtggtgctgttgacacatggtttaatgctctcacgaaagaggcgccacctactgaacagtcaacatcgaaaccgaaacgaggtcttttcggacgcaaaaagtccactagtgaagctcgatcgtctggtactgcattgaacaaggtccggagttctggctcaatagatgggaacatggattgggaagccaatcttgtcttcaacacgagtttatctgctggggctcccatgtcacctcttacttcgcaacaagctcaggcattactcagcgaccttcccgtactgcaacaaatatggctcacaacagctgaggccctgatccttcagcgtcgggttgtcgatggtgttgcccaggcccctcagaaggacaccgtcaagggcaaacccgaggcaactgactctctgatccggctgctcgttattgcctttgcccctattgctgttaacgcaggtgtcttggctgctacgttcggcatggcctgctgtatgggtcccctactcagcatgtgctgcaagaagtttggcagtgttctcgccggtatcgctcacgctactgctgttatattcttgttgatctccttcctgggtatgtttgttctcgagggcttcaactttacccgcacactgtctggtatgatcgccgtcacttgcatccagcgctttttcgtcaagctcattgtgtctcttgcccaagcttctagaaac

SEQ ID NO: 222 - DNA, 1723 bp, synthetic construct
  source 1..1723: mol_type = other DNA
ggtaccgaattctaatacgactcactatagggcaagagacacaatgagcttgacgaaaaagcgctggatgcaagtgacggcgatcataccagacagtgtgcgggtaaagttgaagccctcgagaacaaacatacccaggaaggagatcaacaagaatataacagcagtagcgtgagcgataccggcgagaacactgccaaacttcttgcagcacatgctgagtaggggacccatacagcaggccatgccgaacgtagcagccaagacacctgcgttaacagcaataggggcaaaggcaataacgagcagccggatcagagagtcagttgcctcgggtttgcccttgacggtgtccttctgaggggcctgggcaacaccatcgacaacccgacgctgaaggatcagggcctcagctgttgtgagccatatttgttgcagtacgggaaggtcgctgagtaatgcctgagcttgttgcgaagtaagaggtgacatgggagccccagcagataaactcgtgttgaagacaagattggcttcccaatccatgttcccatctattgagccagaactccggaccttgttcaatgcagtaccagacgatcgagcttcactagtggactttttgcgtccgaaaagacctcgtttcggtttcgatgttgactgttcagtaggtggcgcctctttcgtgagagcattaaaccatgtgtcaacagcaccacgaatcactaggtttgggcgttgataaacagataggggagaatcgtgacaacggcaagcatgagaggagagaaaatctcgctaaagaagatgttggcgaacggagcgcgaggaacatccgcagacatcttagcagaggggtctcccagagccgtcatcagtgacaccgacgggaggccgcacagaatcgggaatgctgcggccgtaccaccattcgtcctcttctacaagtccatcttcactgatcaaatccagcatgacctgttggttgcgtttgatatcctgtggacgtttagtgatttgtggtgttgttgatatatggtttaatgtttttatgaaagaggtgttatttattgaatagttaatattgaaattgaaatgaggtttttttggatgtaaaaagtttattagtgaagtttgattgtttggtattgtattgaataaggtttggagttttggtttaatagatgggaatatggattgggaagttaattttgtttttaatatgagtttatttgttggggtttttatgttattttttattttgtaataagtttaggtattatttagtgatttttttgtattgtaataaatatggtttataatagttgaggttttgattttttagtgttgggttgttgatggtgttgtttaggttttttagaaggatattgttaagggtaaatttgaggtaattgattttttgatttggttgtttgttattgtttttgtttttattgttgttaatgtaggtgttttggttgttatgtttggtatggtttgttgtatgggttttttatttagtatgtgttgtaagaagtttggtagtgtttttgttggtattgtttatgttattgttgttatatttttgttgattttttttttgggtatgtttgtttttgagggttttaattttatttgtatattgtttggtatgattgttgttatttgtatttagtgtttttttgttaagtttattgtgttttttgtttaagcttctagaaac

SEQ ID NO: 223 - DNA, 1726 bp, synthetic construct
  source 1..1726: mol_type = other DNA
ggtaccgaattctaatacgactcactatagggcaagagacacaatgagcttgacgaaaaagcgctggatgcaagtgacggcgatcataccagacagtgtgcgggtaaagttgaagccctcgagaacaaacatacccaggaaggagatcaacaagaatataacagcagtagcgtgagcgataccggcgagaacactgccaaacttcttgcagcacatgctgagtaggggacccatacagcaggccatgccgaacgtagcagccaagacacctgcgttaacagcaataggggcaaaggcaataacgagcagccggatcagagagtcagttgcctcgggtttgcccttgacggtgtccttctgaggggcctgggcaacaccattttgggcgttgataaacagataggggagaatcgtgacaacggcaagcatgagaggagagaaaatctcgctaaagaagatgttggcgaacggagcgcgaggaacatccgcagacatcttagcagaggggtctcccagagctggtgttgcccaggcccctcagaaggacaccgtcaagggcaaacccgaggcaactgactctctgatccggctgctcgttattgcctttgcccctattgctgttaacgcaggtgtcttggctgctacgttcggcatggcctgctgtatgggtcccctactcagcatgtgctgcaagaagtttggcagtgttctcgccggtatcgctcacgctactgctgttatattcttgttgatctccttcctgggtatgtttgttctcgagggcttcaactttacccgcacactgtctggtatgatcgccgtcacttgcatccagcgctttttcgtcaagctcattgtgtctcttgcccgtagtgattcgtggtgctgttgacacatggtttaatgctctcacgaaagaggcgccacctactgaacagtcaacatcgaaaccgaaacgaggtcttttcggacgcaaaaagtccactagtgaagctcgatcgtctggtactgcattgaacaaggtccggagttctggctcaatagatgggaacatggattgggaagccaatcttgtcttcaacacgagtttatctgctggggctcccatgtcacctcttacttcgcaacaagctcaggcattactcagcgaccttcccgtactgcaacaaatatggctcacaacagctgaggccctgatccttcagcgtcgggttgtcgacgtcatcagtgacaccgacgggaggccgcacagaatcgggaatgctgcggccgtaccaccattcgtcctcttctacaagtccatcttcactgatcaaatccagcatgacctgttggttgcgtttgatatcctgtggacgctcgacaacccgacgctgaaggatcagggcctcagctgttgtgagccatatttgttgcagtacgggaaggtcgctgagtaatgcctgagcttgttgcgaagtaagaggtgacatgggagccccagcagataaactcgtgttgaagacaagattggcttcccaatccatgttcccatctattgagccagaactccggaccttgttcaatgcagtaccagacgatcgagcttcactagtggactttttgcgtccgaaaagacctcgtttcggtttcgatgttgactgttcagtaggtggcgcctctttcgtgagagcattaaaccatgtgtcaacagcaccacgaatcactacgtaaagcttctagaaac

SEQ ID NO: 224 - DNA, 1726 bp, synthetic construct
  source 1..1726: mol_type = other DNA
ggtaccgaattctaatacgactcactatagggcaagagacacaatgagcttgacgaaaaagcgctggatgcaagtgacggcgatcataccagacagtgtgcgggtaaagttgaagccctcgagaacaaacatacccaggaaggagatcaacaagaatataacagcagtagcgtgagcgataccggcgagaacactgccaaacttcttgcagcacatgctgagtaggggacccatacagcaggccatgccgaacgtagcagccaagacacctgcgttaacagcaataggggcaaaggcaataacgagcagccggatcagagagtcagttgcctcgggtttgcccttgacggtgtccttctgaggggcctgggcaacaccattttgggcgttgataaacagataggggagaatcgtgacaacggcaagcatgagaggagagaaaatctcgctaaagaagatgttggcgaacggagcgcgaggaacatccgcagacatcttagcagaggggtctcccagagctggtgttgtttaggttttttagaaggatattgttaagggtaaatttgaggtaattgattttttgatttggttgtttgttattgtttttgtttttattgttgttaatgtaggtgttttggttgttatgtttggtatggtttgttgtatgggttttttatttagtatgtgttgtaagaagtttggtagtgtttttgttggtattgtttatgttattgttgttatatttttgttgattttttttttgggtatgtttgtttttgagggttttaattttatttgtatattgtttggtatgattgttgttatttgtatttagtgtttttttgttaagtttattgtgttttttgtttgtagtgatttgtggtgttgttgatatatggtttaatgtttttatgaaagaggtgttatttattgaatagttaatattgaaattgaaatgaggtttttttggatgtaaaaagtttattagtgaagtttgattgtttggtattgtattgaataaggtttggagttttggtttaatagatgggaatatggattgggaagttaattttgtttttaatatgagtttatttgttggggtttttatgttattttttattttgtaataagtttaggtattatttagtgatttttttgtattgtaataaatatggtttataatagttgaggttttgattttttagtgttgggttgttgacgtcatcagtgacaccgacgggaggccgcacagaatcgggaatgctgcggccgtaccaccattcgtcctcttctacaagtccatcttcactgatcaaatccagcatgacctgttggttgcgtttgatatcctgtggacgctcgacaacccgacgctgaaggatcagggcctcagctgttgtgagccatatttgttgcagtacgggaaggtcgctgagtaatgcctgagcttgttgcgaagtaagaggtgacatgggagccccagcagataaactcgtgttgaagacaagattggcttcccaatccatgttcccatctattgagccagaactccggaccttgttcaatgcagtaccagacgatcgagcttcactagtggactttttgcgtccgaaaagacctcgtttcggtttcgatgttgactgttcagtaggtggcgcctctttcgtgagagcattaaaccatgtgtcaacagcaccacgaatcactacgtaaagcttctagaaac

SEQ ID NO: 225 - DNA, 3267 bp, synthetic construct
  source 1..3267: mol_type = other DNA
atggaggagaagccagcggcggtcaatcgccagagtgcgatcccccgcatgtcacgacttccaacgccaaggtcgacatcatcttccaacctccgaccagctccttcgaaagagaacttgaaccctgtcccgaagcagcgccttcgcccagcagcttctcgagatcaattgccccgagcctcatcgattccttccaatagacttgaaccccgcgaagccttggccacgacgcctcgcccgagacgagacatgtctgccgccagaccacgagccacggattcaagaccgagcttagctgcctcgactacaccgactcactctccctcgcagccgcctttgagccataccccaacaggcgatcccccctcggacccagaccacttcctcttcaagaaaccgacagccctctcacgacgcccctccgaagctcaattgtccacgccgagcggtgctatgaaccagtcacctgtcgatagtgaaccgcccatcaatgacagcactgacagcatgtcgttcgggaccatccgacctaggtcttcgaagccaaggccgtctttgtccgaacgaaccatggagacactagcgcagctcccctcttccccggcaatgagcaagaaatcgacctccttctttgacccggacggtacaagaaggtctcggtcgcgggcaggaagcaatggatcgaggccaggcttgagctatacatccgatggatcgatgcgtccgccctcgcaggcaagaagccgcccagggtcaagctctggccctgacgatggcagtgtctcgagcttcagggcctctacgaatacttttcgacagcctctgagcacaattgagggtacgccatcacggcgtacctcgggcattgcttcgctgaggacgccctctgtccgaggcgtcgctcgcgccaccccagcccgctcgacgaacttctcggcctccacgatcggacattccttcgatagaggttcgagtccaaccaagtcaaccggtattgcagcaccgccaaagactggggccaagacaatagcggctcggccgctgaagcagagacagtcgatcaacggtctcttcaagaagccttcgctgcctgcgatggaccgcactgccgctgcacagccagctcctcccaagtttacaccgaagccggcacccaagacgacaccgacaccatcaaggctcacaaagccaactcgaacgataccaaagccaggagcaactggtgctccatcggaagccattgggaagccggatatggaggccagtcgaaaatcgtccaccgcgcttcgcgagcagattgcacaggctaaagctgcaaagcgcgctgcggtgaagaaggctcccgatctggacctggtcccagactcgttggccgtgacagatgatccaaagtccttcccgctcaaatccccgattgtgccagccgacaacagcttcgatttcgggatcgccgtaacgcatgacccattcaatacacagagaacacagagcgcaaagaacaagattgtgcaacagcgtctcggtgatgcgcgcagctctggcaggctcaatatcgcggccatgggactgagtgtcatgcctgtcgaagtactgaaaatgtatgatgctgagatgatggggactaatgacggaagctgggctgagagcgtcgatctgacgaggctgattgccgccgacaacgagatggagctcatcgacgacagcatcttcccagacgtgactgccgacgacctagcagctgaggaggacgggaatgggaacatttttggcggactggacacaatcgacctgcacgggaatctattgatctctcttcctgtcggactgaggcagcttcgcctcctgacctttgaattgctgccatttggcattctgtcccagatgcccttgacagaactcaacctgaagaagaacaagcttgcaaagacgctcattgaagacgacgtggaggctctcccgaagctgcaatcaatcgacatctcctgcaaccagattacgcatatcgttgcccctggcacaacggtcagcctgccggtgctgtatcagctcagcgcgtctatgaaccgcctgaaagctctgcctgacgtcagctcttggagtagtcttctgacgctgaccgcggacgagaacagtatctcggagtttccggaaggcttctttagtctggagaagttgcgacatgttgatttctctgctaatgatattcgcatcatcccgccggagattgccaggatggacaatctcgcaatgattcgcctgagtggcaacccgttgcgtgacaaaaagttcatctcggccacgacagaggagctgaaagatgccctggcagcgaggctggaaccgccgcctccttacgacgatgtcacaagcaccgcgcacacaatcgcggctgccatcgctgacgatgccaaagcatccgaatcaccaacgaaagaagtcgcacctgaagagggtgagcatgacagtcgatccgacatggatgacttcgcaactcccccgacatcggcgccgcactcgcctgctcgatcgaggtcgcacaccctttcgagccaaacttggcccgttaagaatggcggactgctggaccgctctggaacacagtcctcgtcgcttcaccctgtcgtttgttccaaaattgccgccgctacccgggtcacggaaatccagctccatcacaaccttttcgcaacgtttcctgaatctcttacgttcttcgccaacaccttgacggcgctgtctttggctcacaacaagcttgtcggcgagacgtacttgccagaggagctggagctacctgctctccgggagctcaatctgatgaacaaccacatcacgagcctggccccgctgacggccaatctccgatcgccgcagctcgagaaagttgatgtctcgctcaaccgtatcacagcgctgccgactgatctacgtgtcgtgttcccgcagctgtcggtcatgcttattgccaacaatcaccttatcgagctggaccctgagagcatacggggtctgcaggtcgtggacgccaacaataacgatatcgcacacttgaaccccaagatcggcttgcttggcggaaacggcggcctgcgtcagcttgacgtatcagggaaccgcttccgtgttccgaggttcagcgtgctcgaacgtggaacagatgcaacactgaggtggttgcggggtcgtgtgcctgttgccgagatggccagctggaaagaaggacaaggagagggtgggcagggcgattctgacgacgacgtcgactga

SEQ ID NO: 226 - protein, 1088 aa, synthetic construct
MEEKPAAVNRQSAIPRMSRLPTPRSTSSSNLRPAPSKENLNPVPKQRLRPAASRDQLPRASSIPSNRLEPREALATTPRPRRDMSAARPRATDSRPSLAASTTPTHSPSQPPLSHTPTGDPPSDPDHFLFKKPTALSRRPSEAQLSTPSGAMNQSPVDSEPPINDSTDSMSFGTIRPRSSKPRPSLSERTMETLAQLPSSPAMSKKSTSFFDPDGTRRSRSRAGSNGSRPGLSYTSDGSMRPPSQARSRPGSSSGPDDGSVSSFRASTNTFRQPLSTIEGTPSRRTSGIASLRTPSVRGVARATPARSTNFSASTIGHSFDRGSSPTKSTGIAAPPKTGAKTIAARPLKQRQSINGLFKKPSLPAMDRTAAAQPAPPKFTPKPAPKTTPTPSRLTKPTRTIPKPGATGAPSEAIGKPDMEASRKSSTALREQIAQAKAAKRAAVKKAPDLDLVPDSLAVTDDPKSFPLKSPIVPADNSFDFGIAVTHDPFNTQRTQSAKNKIVQQRLGDARSSGRLNIAAMGLSVMPVEVLKMYDAEMMGTNDGSWAESVDLTRLIAADNEMELIDDSIFPDVTADDLAAEEDGNGNIFGGLDTIDLHGNLLISLPVGLRQLRLLTFELLPFGILSQMPLTELNLKKNKLAKTLIEDDVEALPKLQSIDISCNQITHIVAPGTTVSLPVLYQLSASMNRLKALPDVSSWSSLLTLTADENSISEFPEGFFSLEKLRHVDFSANDIRIIPPEIARMDNLAMIRLSGNPLRDKKFISATTEELKDALAARLEPPPPYDDVTSTAHTIAAAIADDAKASESPTKEVAPEEGEHDSRSDMDDFATPPTSAPHSPARSRSHTLSSQTWPVKNGGLLDRSGTQSSSLHPVVCSKIAAATRVTEIQLHHNLFATFPESLTFFANTLTALSLAHNKLVGETYLPEELELPALRELNLMNNHITSLAPLTANLRSPQLEKVDVSLNRITALPTDLRVVFPQLSVMLIANNHLIELDPESIRGLQVVDANNNDIAHLNPKIGLLGGNGGLRQLDVSGNRFRVPRFSVLERGTDATLRWLRGRVPVAEMASWKEGQGEGGQGDSDDDVD

SEQ ID NO: 227 - DNA, 5472 bp, synthetic construct
  source 1..5472: mol_type = other DNA
atggcgcacgcgtggctcgactctctctccgaggactggccctcgcaactgggctcagatgcctcgccgccccctcaactccctccactcaattcgaacgagaacagcgcggttgccaatggaagggcacctcctagcaggctgccactgcccatgaagacggcgccaacagccaatgccaatgacggcagcttcaatatcctcagcgagcgtagcaccaacgagaggaatcttccctcctccgcgcgcaaaccctccaaactccccaatgagatcgacgccggtcccgagagccgctatgcgagccgaacgttctctgcctcgaccaccgggtctgtggtccataacacggtaaaccaccgatcgacaagcgcatcacccaacaaaaagggccacacgccagaatggaagcgacgtctagtctatggggaggtgccctacggcgagcagcaagatcttttccaatccgcggccaccgggctagagaatatgtttaagccacccccaccaccacaagaaggctctgtcgtcgacgacagtcgacaacactcgggcatgccttccagcccacctatttatcgcccggccgaacctgctacaccggtatatgacgacgatgacgacgatgaaggcagcgttgtgcatgcgatggagggtagtcccagccccagcccacggaagcctcgagaaatgacatacaaaatggtcgacgagtcgtatgatgcgggcagtctcgacagctatagcacacaaggacacagtcaagaggctgctgactcgatagctgaagagagcctatcggtgcgcgtagcatctgccaacggtcttgcgccaccgaccgatgcgtcaaggaaaaccagcggacagagcgttgttcgtaatgaggacttcagccccatcgtgctcacgaagcaggtcggcaatgtcggccaggttgactttgcacctgtcgaagtaccggccgatcagctaaagaatcgtttggaaaaactgcgcatcaaccagatgctgctggactcgtacgccgagggagagctcgacttcagcgacctgcaagaggaaggcatctcgggaggtcccgaagacactgacgaatttgcgcgacagggtggtttcttgaacactcggcgtggcggccgatctgctgagggatctttccgacatcgcctgctcagcccaggcatgtctggcgacacgtctgacatgctcccggaggactcgcttcaagctagcacccccaagcaattccccaccatccgcatcaacgaggaaggctttggcagccatcgtttcgcctctgagacgccgcctcagcttcgcgcgccctttccaagtcccgagaaacaggacaccaaccctcagtccaacggcggaagcccgctgaagctcttcgggccatacgacacgttcactaaccagactcttctacgccgcatcagccagtttgaagacaactcgtcaaacgaatccaggctatctgaggaacagggagaagaaatgcaggcttatagccagagtctggcggcaccctccagcccgccacgacccaaaccctcccaattcatgccaagcgctcgatcggtcagtcagttcggccgaggcgatttgcagggttatcagttcggcgatgtgtcactccgatctgatggcgcaacctcggaagaggatgaaagccatgtcagtgtgcctgcaacatctagccgcaacatgaagtttgatctcgagcctacatctccacaaggcgatgtacccctcatcatcccaaacaaacggcggcaacagggcgtgacgccatcgtcctcgagaaacaacagaacagtttccttttccagtcgcgtcagtgttcagtaccacgacagcagcaggcccagcagcagaccgggcccaccggtgctggcaacgcccaagcgagacagcggctccgaaggcaagcggcctcggacgtcacccaccaaggatccaacccccaagcgaagacgtactctccacaagtccgatgtagcctacggccacgagcagctcgccgcgcttgaaacggtgcaggattcgcattggtacatgcaatccatgatgggcaagcgccaggaggttcgttcaggcggattccagcagctggcccaccctgacgtcctcgccgcccgacagatgagccggccacgcacgccaacgcctagtcagcgatcctcggtgcagcgtgaaagacacccgagctctgaaatccaacctcaggagcagtcacagtcgcagtcgcaatattccccaaagcgcagctcgcagctggcgaatggctcgatggaacggaacgaaagcaccgaaactgatcgcaagccatccatcaagacgcaggactttgtcgagcaggctggtgccatcatggcgatgattcgcagcggaatcaaaccacccaatggactcgacagcgtggaggagtcagaggagggcgattatgccagcccaagccctgatatccaggacgtgtcgtttgaggagtcgacgagggaacctctttcccggccaccaagccgcgacggcaagccggtgacaagagttccgccccgccaggagaaccccgaggtgctggattacctgaagaaatatcaggagctcagtgacatgggcgatgtcatctcctcatctctgcggtctatgggtctggcccaggatgctatccgtgccgctcaagaagtggagaagatggttcaagcgggccaggacgtttcctatcccgacgatctggattctttggaagacatcatcagtgacccgcctaatattcgaatcagccgcggaccagccgccgaccaaaacgcaatcggaccagactacccgtccaacatctcggatggctcaacaaaaagcatccccaccgggtcgtcacggggttctgagtccaagaagaccattctgccagcgagcgtcgttcacctgcttcccgagtctgtcgcgggcatgtatctcgacaaacagcaaaatatctgggtcaagcgcagggctgagtctgtccgcatccacaacattctgccttccgaagacagcgaggatgacccgtttgcgagcatccccgacctgaccgttgacatgacgagggagatggagaacctgcgtctgaatccctcacagcaaaccctcagtggcgacgagaacgagtcctctggagactattcacgcacgccgagcagccagcgcagcaagagctacgtcaccctgtccccgaacggtgcgatacctgagggtatgtctccccaggcgcaagaggagctcacgaagctcgagagcatggtcaacagcccaactagcgatcgcgacgactacgaggaggagatgcgcgtcgaagaccgcaaccgcgtgtccccgtcgaggaagaacctgaccatttccttctcttcgcctattgcatccatcattcacgacgtggttcccgaggacattgatagactcgaggacgacacgggtagcatctcgcatgccgacggtggatcatggtctgagcaacagaccgataaccagtatcgcagtctcacgaagaatgcgcgcaagaagttgctggctcggtccaaaaacggcacgcggagggcgtctcgaaacctcgcggttcaaggacaggcctttacgccacgtccagtgtctcgcatcgacgaacaggatgaagacagccagaacgagcgcgcccacgccgagagcaggcaaatcagcatcctcgctgatgcaagcgttgtcagccaccaggatcgccagacaagcctcagcttcgtagtgtcgacacccgccgctcaccacggtgccggagccatcatcagccaaaacgtcggcaacctgtctctcagccctctgtccgagttcacgatgaacaaccaggaccaatcgttcggactggaggttagctacctggtgcaggaccgacacttggtcacaggcgatggttctaagaaagtgctgtccatgacggtccgcgaccttgtcgaccgactgaccgaggtggagcctttcgaggccaactgggacgactttgtggaactggacgtcagcgacaaacgcttatcgagcctgcatatgctggatgagttttgcgggcgactggtgtcactggacgcctcgaccaacgcactgtcacatcttgatggcgtgccctcgacggttcgccacctcaaggcgtccagcaacctgttgacggagctgacttcttgggaccatctgtcaaatctacagtacattgacgtgtcaaacaatgagctgcgcagcctggctggcctcaacaatctcgttcacctgcgaggcgtccgcgcggataacaataagctgacatcgctggacggcttccacttccacgacgggctcctgtcgctgcgagcgcgggacaacctgatcgaggagctcgattttgacggcaccaagcttgaccggttggcagagctggacctgtgcggcaacaagatccagtcgattcgcaacctgggacaactcccgtcgctggagactctcaaggtgcagcgcaaccagcttgtggacttacacaacgaaggaccactgccatgccttcgacaccttgacattggggacaacaatgtgacatcgctcgacatcagctctatgccttgcctgcgcgttctgcacgctgataggaaccgcattagcagaatcactggcttcgcgcgtgcaaggtatctagacagcatatcgctcagagagcagcgcggcgacgagcctctcgggctagcgttcctgtccgccgcctatgaggtgcgcaagcttttcctgtcgggcaactacattggtcaattcgagccgcaggtcgatttcctcaatctgcagcttcttgagctggcaaactgcgggctgcagtcactcccggagaatctcggtcagctgatgccgaacttgcgatcgctgaacctcaatttcaacgccattgcggacatcagcccgctgcgattcattccgcggttgaagaagctgctcatggcgggcaaccgactggccaactcgacgcaagtcacggaggtggtgaccgagttcccgcacctcagcaagctggatctgcgggacaacccgatgacacagggcttctacgcgccactgcaaggcatggtgcctgtggatcgcacggggtatgtggatccttttatgctgcccgaggcagatgccgagcgggacgcacttttcgcgggccgactggaccaagcgacacgactgcggcggcgtctgtaccaggtggtgtttgtcgggtgctgcgctcggctgaaagagctggacgggctgcccgtcaagcgcagggaggcgctggccagggatgcagcgtacgagatgctagtgaaggagggtctcctaccggggcagcagcagtccgaagaaacgggggctttggagccgaggcgcaagggtggggagcagcaagccaaaccgcacgagcaagcagaggctgcaaaatgcagcgatgacagcagtcggtggaatgcggaagacagctttgcctga

SEQ ID NO: 228 - protein, 1823 aa, synthetic construct
MAHAWLDSLSEDWPSQLGSDASPPPQLPPLNSNENSAVANGRAPPSRLPLPMKTAPTANANDGSFNILSERSTNERNLPSSARKPSKLPNEIDAGPESRYASRTFSASTTGSVVHNTVNHRSTSASPNKKGHTPEWKRRLVYGEVPYGEQQDLFQSAATGLENMFKPPPPPQEGSVVDDSRQHSGMPSSPPIYRPAEPATPVYDDDDDDEGSVVHAMEGSPSPSPRKPREMTYKMVDESYDAGSLDSYSTQGHSQEAADSIAEESLSVRVASANGLAPPTDASRKTSGQSVVRNEDFSPIVLTKQVGNVGQVDFAPVEVPADQLKNRLEKLRINQMLLDSYAEGELDFSDLQEEGISGGPEDTDEFARQGGFLNTRRGGRSAEGSFRHRLLSPGMSGDTSDMLPEDSLQASTPKQFPTIRINEEGFGSHRFASETPPQLRAPFPSPEKQDTNPQSNGGSPLKLFGPYDTFTNQTLLRRISQFEDNSSNESRLSEEQGEEMQAYSQSLAAPSSPPRPKPSQFMPSARSVSQFGRGDLQGYQFGDVSLRSDGATSEEDESHVSVPATSSRNMKFDLEPTSPQGDVPLIIPNKRRQQGVTPSSSRNNRTVSFSSRVSVQYHDSSRPSSRPGPPVLATPKRDSGSEGKRPRTSPTKDPTPKRRRTLHKSDVAYGHEQLAALETVQDSHWYMQSMMGKRQEVRSGGFQQLAHPDVLAARQMSRPRTPTPSQRSSVQRERHPSSEIQPQEQSQSQSQYSPKRSSQLANGSMERNESTETDRKPSIKTQDFVEQAGAIMAMIRSGIKPPNGLDSVEESEEGDYASPSPDIQDVSFEESTREPLSRPPSRDGKPVTRVPPRQENPEVLDYLKKYQELSDMGDVISSSLRSMGLAQDAIRAAQEVEKMVQAGQDVSYPDDLDSLEDIISDPPNIRISRGPAADQNAIGPDYPSNISDGSTKSIPTGSSRGSESKKTILPASVVHLLPESVAGMYLDKQQNIWVKRRAESVRIHNILPSEDSEDDPFASIPDLTVDMTREMENLRLNPSQQTLSGDENESSGDYSRTPSSQRSKSYVTLSPNGAIPEGMSPQAQEELTKLESMVNSPTSDRDDYEEEMRVEDRNRVSPSRKNLTISFSSPIASIIHDVVPEDIDRLEDDTGSISHADGGSWSEQQTDNQYRSLTKNARKKLLARSKNGTRRASRNLAVQGQAFTPRPVSRIDEQDEDSQNERAHAESRQISILADASVVSHQDRQTSLSFVVSTPAAHHGAGAIISQNVGNLSLSPLSEFTMNNQDQSFGLEVSYLVQDRHLVTGDGSKKVLSMTVRDLVDRLTEVEPFEANWDDFVELDVSDKRLSSLHMLDEFCGRLVSLDASTNALSHLDGVPSTVRHLKASSNLLTELTSWDHLSNLQYIDVSNNELRSLAGLNNLVHLRGVRADNNKLTSLDGFHFHDGLLSLRARDNLIEELDFDGTKLDRLAELDLCGNKIQSIRNLGQLPSLETLKVQRNQLVDLHNEGPLPCLRHLDIGDNNVTSLDISSMPCLRVLHADRNRISRITGFARARYLDSISLREQRGDEPLGLAFLSAAYEVRKLFLSGNYIGQFEPQVDFLNLQLLELANCGLQSLPENLGQLMPNLRSLNLNFNAIADISPLRFIPRLKKLLMAGNRLANSTQVTEVVTEFPHLSKLDLRDNPMTQGFYAPLQGMVPVDRTGYVDPFMLPEADAERDALFAGRLDQATRLRRRLYQVVFVGCCARLKELDGLPVKRREALARDAAYEMLVKEGLLPGQQQSEETGALEPRRKGGEQQAKPHEQAEAAKCSDDSSRWNAEDSFA

SEQ ID NO: 229 - DNA, 1668 bp, synthetic construct
  source 1..1668: mol_type = other DNA
atgggcgaaccacttgccaaccgtaagaagttcaacactgagctgacggactactcttcccaacatgctgagaaaaccgggccgtacgccgagaacctggaagttgatgccctcatcgtcggtgccgggttcgctggcattttcatgctcaagactctccgggatcggggctataagtctgtgatcttcgaagctggtaacgacaccggcggcacatggcgatggaactgctaccctggcgccggcgttgactcagaggtgccagagtacgagttttcgtggcctgaggtctggaaggactggaactggtcgagcaactaccctaggtacgacgaacttcgagcctattttgaccatgtcgacaaggtcctcggcatcaagaaagactgcgccttcaacacggttgttgtgggtgcacactttgacactgccaccggtcgttggaacgttcgcacggcggatggccgagtgaccaaagcccgcttcctcgtcctcggcaccggctttgccgcccgacgctacattcccgactggcctggcatggacaagttcaagggcgtggtccaccactcctcgttctggcccgacgaagaggtcaacgtcaagaataagcgctgtgctgtcattggcaccggcgcctcgggcgttcaaattgtccagaattggggtcccaaggccggccaggttactgtcttccagcgtactccgaaccttgctgtgcccatgcggcgccgccagctcacagtcgccgagcaagagcgcgccaaggtcgtgtacccggagctgtttaagctacgcgagacgagctttgccgggtttcactacgactggatcgagaagaacacctttgacgacacgcccgagcagcgcgaggccacgtacgagcgagtctgggcagagggcggcttccggtactgggtggcgctctacaaggataacctgttcaaccctgaggccaacgaggcctcgtatgcattctgggcggagaagacgagggcgcgcatcggggaccctcgcctgagagatttgcttgcgcccttggtcatgccgcactacttcggtgtcaagagaccttgcctcgaggatgactactttgagcagttcaaccgcccttcagtcgatctagtcgatatcagcaagaacggcatcaaggagtttaccgagacagggatcactctcgaggacgggacacatcaagagttcgacgtcatcgccatcgccaccggctttctcggcctcgagagcatcgatggcaacaagctccaagaagaatgggtctccggtgccaagacatacctcggcaccaccgtcagcggctatcccaacatgttccatatctacggcgtccatggtccaaccctcctgagcaacggcccgtcgacagtcgaggttcaaggccgctggatcgctgacatgattgacaagctgcagcacaatgatatccggtacatcaatcccaagcccgaggccgctgacaagtggaaggagcacatccttgagctgaataacgagtcgttgttcccaacgacgcgatccacctacatgggcggaagcatacccggaaagaaggttgaacctgtgtgctacacgggtggtattcctgcgtataagattgagatacgcaaagcgctaaacagtatggagggctttgagatcgtcaaagggtga

SEQ ID NO: 230 - protein, 555 aa, synthetic construct
MGEPLANRKKFNTELTDYSSQHAEKTGPYAENLEVDALIVGAGFAGIFMLKTLRDRGYKSVIFEAGNDTGGTWRWNCYPGAGVDSEVPEYEFSWPEVWKDWNWSSNYPRYDELRAYFDHVDKVLGIKKDCAFNTVVVGAHFDTATGRWNVRTADGRVTKARFLVLGTGFAARRYIPDWPGMDKFKGVVHHSSFWPDEEVNVKNKRCAVIGTGASGVQIVQNWGPKAGQVTVFQRTPNLAVPMRRRQLTVAEQERAKVVYPELFKLRETSFAGFHYDWIEKNTFDDTPEQREATYERVWAEGGFRYWVALYKDNLFNPEANEASYAFWAEKTRARIGDPRLRDLLAPLVMPHYFGVKRPCLEDDYFEQFNRPSVDLVDISKNGIKEFTETGITLEDGTHQEFDVIAIATGFLGLESIDGNKLQEEWVSGAKTYLGTTVSGYPNMFHIYGVHGPTLLSNGPSTVEVQGRWIADMIDKLQHNDIRYINPKPEAADKWKEHILELNNESLFPTTRSTYMGGSIPGKKVEPVCYTGGIPAYKIEIRKALNSMEGFEIVKG

SEQ ID NO: 231 - DNA, 2630 bp, synthetic construct
  source 1..2630: mol_type = other DNA
aacatgaacaacccataagctccctccttcttcatcctgccccctcccgccgcgatcttcgcgaccgcttctgttgctcgcttttgcgttttctttctcttgctctccttcttaggcaatcgacgagagattttccctcccgcgggactttaatacccacgagcctcggtcgcaaacccccttattctgtcagccgcctggtgcaggcattgtgcaaagcccccttcgatctctcgccgtccgctgcggcaacttaggcgcaagcacgggctacgtccaattcgattcatttaccttcattgtatatctcttttacgactgttcagccggctctgaatcgactattacatcacccatttggatatatggcgccgcctgcactctcgcttgtctaaacaccaactgaacaacgacaccgaccggttcgcaaacggacatatacgggcagaacctcgcaataatagaagcaagagacaggggaaagagagctgacgggggaaactgcatacaacgggacatccgttctgagcgacattgtccttgagggacggtcataccagctatatcccgactcgtcagcacgcagacgattatagcactaccagcacgatggactactactcgatgggctcagagaagccctcggagcgttcaagatggacgccactgacacgcatgctgctctcgggagagatgacgcaggaaaagcagaagcagctgacatcgagggagaaattcgatcgctggatgatcaacgagggttaccgaagattttgcccaagttttgtcttcgtcttcatgatcctccacggactcgtctttgcgcttgcctgcgtcaactttgccctcaaggacaatctgcaggttgcacgagacatattcggtccaacttttgtcatcgcccgtgccgcggctctcgtcctacacgtcgatgttgccctcgtactctttcctgtctgtcgcacattcatctcgctcgctcgccaaacgcctctcaatggcattatccagttcgataagaacatcaccttccacaagacgattgcctggtccattgtctttttctcatgggttcatacgattgctcactgggtcaactttggcatgatcgccgccaagaacaaccttggcgtctatggctggctgctggccaacttcgtctccggtcccggttggactggttacgttatgctcatcgccctcatgggcatggttatcacgtcaatggagaagccccgacgcgccaactacgagaggttctggtacacccaccacatgtttatcgttttcttctttttctggtcaattcacggcgcattctgcatgatccagcccgattttgcgcccttctgcatcagtatcggcccgagcgcgatcggcgtcttctggcagtactggatgtacggcggtttctgctaccttgctgagcgcatcgcccgtgaggtgcgtggcaagcataagacatacatctccaaagtcattcagcaccccagcaatgtctgcgagattcagattaagaaagaaaacaccaagacccaggccggccagtacatcttcttctgctgccccgccgtatcgatttggcagtaccacccctttaccctaacgagtgcgcctgaggaagattacatctctatccacatgcgcgtagttggtgatttcactcgtgctgtctctaccgtattgggctgcgagttcgacaagaagaaggacggtagcaaggtcgtcggtgtcaatggcgatggaagcgatgtcgatcctgctttgcgccgcgtgctgcctcgcgtttacatcgacggaccctttggatcggcttcggaggatgttttcaagtacgagatttctgttctctgcggcgcaggaatcggcgtcacgccgtttgcatccattctcaagtccatctggtatcgcatgaactatcctcagaagaagacgcgcctcagcaaagtttacttcttctggatctgcagagatttcggctcctttgagtggttccgctcgctgttgctcgccattgaagcgcaggacgttgacaaccgcatcgagatccatacctacctgaccgccaagatcaaggcagacgacgcaacaaacatcatgatcaatgatgccaacgctgacaaagacaccatcaccggtctgaggtcgccaacgaactttggacggcccaactgggacatgatcttccgtggaatcagaaagctgcactcgcctgccgaggctggtgtgttcttctgtggtcccaagggcctcggcagtacactgcatatcttttgtaacaagtacagcgaaccaggcttttcgttcaaatggggcaaggagaacttctagagggggcaaggcgtggagcaattttatttctctattctcatggttttcttcaggttgcatgtctgattgtttgctgatcagtatatggtctgagacttgttagccatgggaatgagcctctcgctgtggctcttattttgttagacgcgggcgaactctacctagcctggacgggtacgcgagggttggaagaggattttctcaaagagaactcgatacatggtcggttgatgttaataggatttatcacgccatatggcgcatcatgattaggatatacaaatatacattacttcttc

SEQ ID NO: 232 - protein, 573 aa, synthetic construct
MDYYSMGSEKPSERSRWTPLTRMLLSGEMTQEKQKQLTSREKFDRWMINEGYRRFCPSFVFVFMILHGLVFALACVNFALKDNLQVARDIFGPTFVIARAAALVLHVDVALVLFPVCRTFISLARQTPLNGIIQFDKNITFHKTIAWSIVFFSWVHTIAHWVNFGMIAAKNNLGVYGWLLANFVSGPGWTGYVMLIALMGMVITSMEKPRRANYERFWYTHHMFIVFFFFWSIHGAFCMIQPDFAPFCISIGPSAIGVFWQYWMYGGFCYLAERIAREVRGKHKTYISKVIQHPSNVCEIQIKKENTKTQAGQYIFFCCPAVSIWQYHPFTLTSAPEEDYISIHMRVVGDFTRAVSTVLGCEFDKKKDGSKVVGVNGDGSDVDPALRRVLPRVYIDGPFGSASEDVFKYEISVLCGAGIGVTPFASILKSIWYRMNYPQKKTRLSKVYFFWICRDFGSFEWFRSLLLAIEAQDVDNRIEIHTYLTAKIKADDATNIMINDANADKDTITGLRSPTNFGRPNWDMIFRGIRKLHSPAEAGVFFCGPKGLGSTLHIFCNKYSEPGFSFKWGKENF

SEQ ID NO: 233 - DNA, 1634 bp, synthetic construct
  source 1..1634: mol_type = other DNA
atgtggaagaacattgtcgagaagatccgtgtcgccgatgaccacgagcatggctacgagacagataaatggatcaatcccgacattgcgcctcttcccccaagccgccgcaactggggaatctgggcctacctagcctggacgggtgccgcctcactcttgtccctgggtctgactgtgcctcagacgattggcctcatggttatcgctcgcatactcatggttgccctcatcattggcaatggatggattggcggagaatggcacattggcttcgccgttcaccagcgcatgatcctcggcatgcgcggggcttacattacgcaagccatgcgaatcatgctgttcatcgtatggtacgggtcgcaggcatggctcggtggtctttgcgtctcagctatgatcagcagctggagctcaagctttctgaacatggagaacacatttgcagcgagcgccaacatggtcactcgtgactttatcgggtttctgctgttccacctcatctcggtgcctttcttgctcatccgccctgaaaattcgaagccctatgtcatcactgcgaacgtgatcgtccttatcgtcatgctggctatcaccatctgggccggcgttaacggtggcactggaccccttttcgtgtcgggcgctcgccagcctggcgtcctctccactggctgggcctgggtctatggtattatcagcaatcttggcgtcatctctgccggcatcgtcaaccaaagcgatttcacccgtttcgcccgccggcagggccttcaagtccctggcatggccttttctctgctggtcccgggaatgattcctttctggaacccactatcagtcatcctgcaatggctcatcgacgactactcgcctggcgcccgcgccgcagcgttcttctgttcgctgggtttcgtgatcggtcagatcgctgagaacatcatgggcaactcgtttgctgcgggcatggacctcgccggtctcttccccaagttcctgaccatcaagcgcggggccctcctatgcgcgctgctgtcctgggccgtgcagccctgggagttctacaacacagcctccgtcttcgtcgccgtcgcagcttctttttccgtcttccttggacccctgacgggcatcatgatcgtcgactacttcgtcgtccgccggcagcgcgtcgagctcagccagctctacacaggctcacgcgacggaagttattggtacacatggggcattcactggcgtgcgctcgccgcgtgggtcatctgcttcgtgcctgccatgccgggcatggtggccaatgtcaaccagagcgtcaccgtgtcagacggcctgcaaaagtacttcctcggcaattatcttttcggtttcgcagaggctgctgtgctgtacacggtgctttgtctcatatcgaggccgaaaaatgcgggtttgcaggacagtgttgacatgtacggcacgtttgacgagaatcaggcgaggaacaagggggtggtgccttttgagcgagtgaaggatgtggatatcccgggcgaggaaccagtgagagaaattgtagagggacaggagaagatacggtacttgagggagtgatgtgatgggtctattgggaggaacaagatgattgtcccgtg

SEQ ID NO: 234 - protein, 530 aa, synthetic construct
MWKNIVEKIRVADDHEHGYETDKWINPDIAPLPPSRRNWGIWAYLAWTGAASLLSLGLTVPQTIGLMVIARILMVALIIGNGWIGGEWHIGFAVHQRMILGMRGAYITQAMRIMLFIVWYGSQAWLGGLCVSAMISSWSSSFLNMENTFAASANMVTRDFIGFLLFHLISVPFLLIRPENSKPYVITANVIVLIVMLAITIWAGVNGGTGPLFVSGARQPGVLSTGWAWVYGIISNLGVISAGIVNQSDFTRFARRQGLQVPGMAFSLLVPGMIPFWNPLSVILQWLIDDYSPGARAAAFFCSLGFVIGQIAENIMGNSFAAGMDLAGLFPKFLTIKRGALLCALLSWAVQPWEFYNTASVFVAVAASFSVFLGPLTGIMIVDYFVVRRQRVELSQLYTGSRDGSYWYTWGIHWRALAAWVICFVPAMPGMVANVNQSVTVSDGLQKYFLGNYLFGFAEAAVLYTVLCLISRPKNAGLQDSVDMYGTFDENQARNKGVVPFERVKDVDIPGEEPVREIVEGQEKIRYLRE

SEQ ID NO: 235 - DNA, 1282 bp, synthetic construct
  source 1..1282: mol_type = other DNA
tgagcctcggaaacagtaccagaccacctactactacatacatctccaccttgacggtacgcctctcgccgaaaccggcagagagagcaatggccacgcaagggttatccgtcaggcgtcgtggcctcgtgccatgacgaacgttcaagttcttcatcatcatcagttgatacctcaacccacctatagctacaatggccccctccgctacctcgattgttgagcccaccgtcctccctcacaagggcggcgtctccgtcggcaacggcattaccaagcctgctgtggcccccaagaccaacagcaactgggatgcctttgccttcgagccgatccgcgagtcgcaggtcgctcgcaccatgggccgccgctacttcgaggacctcgacacctacgccgagtctgacgtcgtcattgtcggtgctggcagctgcggtctctccgccgcctactcgctcgccaatcagcgccccgacctgaagattgccattatcgaggctggcgtcgctcccggcggcggtgcttggctcggcggtcagctcttcagcgccatggtcatgcgcaagcccgccgacaagttcctcgccgacctcggcgtcccctttgaggacgagggcacccacgtcgtcgtcaagcacgctgcgctcttcacctcgacccttttgtcacgtgtcctgtccttccccaacgtgaagctgttcaacgctaccaccgtcgaggacctcattactcgccgtgacggagacgccattcgtgtcgctggtgtcgtcaccaactggaccctcgtcgccatgcaccacggcgaccagagctgcatggaccccaacaccatcaacgctcccgtcatcctcagcaccaccggtcacgacggcccctttggcgccttctgcgccaagcgcctcgtgagcatgcaggccattgagaagctcggtggcatgcgtggtctggatatgaacagtgccgaagatgccattgtcaagggcacccgcgaggtcgttcccggcctgatcattggcggtatggaactgagtgaggttgacggcgccaaccgcatgggccctacgtttggtgccatggtcatgtctggtgtcaaggctgccgaggagatcattgccgtctacgacatccgcaagaaggagaacgagatctagatgctctcgcacttggttcgcgcggtccgcatttgctagtgcacgccgaacaagattggaacatgtctgatggctagcactcactcttgagcgtgtaacacgcacaacgtcgaaactgtcgctctttaaac

SEQ ID NO: 236 - protein, 318 aa, synthetic construct
MAPSATSIVEPTVLPHKGGVSVGNGITKPAVAPKTNSNWDAFAFEPIRESQVARTMGRRYFEDLDTYAESDVVIVGAGSCGLSAAYSLANQRPDLKIAIIEAGVAPGGGAWLGGQLFSAMVMRKPADKFLADLGVPFEDEGTHVVVKHAALFTSTLLSRVLSFPNVKLFNATTVEDLITRRDGDAIRVAGVVTNWTLVAMHHGDQSCMDPNTINAPVILSTTGHDGPFGAFCAKRLVSMQAIEKLGGMRGLDMNSAEDAIVKGTREVVPGLIIGGMELSEVDGANRMGPTFGAMVMSGVKAAEEIIAVYDIRKKENEI

SEQ ID NO: 237 - DNA, 777 bp, synthetic construct
  source 1..777: mol_type = other DNA
gaattctcgagtaatacgactcactatagcgtcgatctgacgaggctgattgccgccgacaacgagatggagctcatcgacgacagcatcttcccagacgtgactgccgacgacctagcagctgaggaggacgggaatgggaacatttttggcggactggacacaatcgacctgcacgggaatctattgatctctcttcctgtcggactgaggcagcttcgcctcctgacctttgaattgctgccatttggcattctgtcccagatgcccttgacagaactcaacctgaagaagaacaagcttgcaaagacgctcattgaagacgacgcgcgtcggcatccggtcagtggcagtgaagggccaacagttcctgattaaccacaaaccgttctactttactggctttggtcgtcatgaagatgcggacttacgtggcaaaggattcgataacgtgctgacgtcgtcttcaatgagcgtctttgcaagcttgttcttcttcaggttgagttctgtcaagggcatctgggacagaatgccaaatggcagcaattcaaaggtcaggaggcgaagctgcctcagtccgacaggaagagagatcaatagattcccgtgcaggtcgattgtgtccagtccgccaaaaatgttcccattcccgtcctcctcagctgctaggtcgtcggcagtcacgtctgggaagatgctgtcgtcgatgagctccatctcgttgtcggcggcaatcagcctcgtcagatcgacgctacgtaactagtctagaaa

SEQ ID NO: 238 - DNA, 777 bp, synthetic construct
  source 1..777: mol_type = other DNA
gaattctcgagtaatacgactcactatagtgttgatttgatgaggttgattgttgttgataatgagatggagtttattgatgatagtatttttttagatgtgattgttgatgatttagtagttgaggaggatgggaatgggaatatttttggtggattggatataattgatttgtatgggaatttattgatttcttttcttgttggattgaggtagttttgtttcttgatttttgaattgttgttatttggtattctgttctagatgttcttgatagaatttaatttgaagaagaataagtttgtaaagatgtttattgaagatgatgcgcgtcggcatccggtcagtggcagtgaagggccaacagttcctgattaaccacaaaccgttctactttactggctttggtcgtcatgaagatgcggacttacgtggcaaaggattcgataacgtgctgacgtcgtcttcaatgagcgtctttgcaagcttgttcttcttcaggttgagttctgtcaagggcatctgggacagaatgccaaatggcagcaattcaaaggtcaggaggcgaagctgcctcagtccgacaggaagagagatcaatagattcccgtgcaggtcgattgtgtccagtccgccaaaaatgttcccattcccgtcctcctcagctgctaggtcgtcggcagtcacgtctgggaagatgctgtcgtcgatgagctccatctcgttgtcggcggcaatcagcctcgtcagatcgacgctacgtaactagtctagaaa

SEQ ID NO: 239 - DNA, 777 bp, synthetic construct
  source 1..777: mol_type = other DNA
gaattctcgagtaatacgactcactatagcgtcggtctggcggggctggttgccgccggcggcggggtggggctcgtcggcggcggcgtcttcccgggcgtggctgccggcggcctggcggctggggggggcgggggtgggggcgtttttggcgggctgggcgcggtcggcctgcgcgggggtctgttggtctctcttcctgtcgggctggggcggcttcgcctcctggcctttgggttgctgccgtttggcgttctgtcccgggtgcccttggcggggctcggcctgggggggggcgggcttgcgggggcgctcgttgggggcggcgcgcgtcggcatccggtcagtggcagtgaagggccaacagttcctgattaaccacaaaccgttctactttactggctttggtcgtcatgaagatgcggacttacgtggcaaaggattcgataacgtgctgacgtcgtcttcaatgagcgtctttgcaagcttgttcttcttcaggttgagttctgtcaagggcatctgggacagaatgccaaatggcagcaattcaaaggtcaggaggcgaagctgcctcagtccgacaggaagagagatcaatagattcccgtgcaggtcgattgtgtccagtccgccaaaaatgttcccattcccgtcctcctcagctgctaggtcgtcggcagtcacgtctgggaagatgctgtcgtcgatgagctccatctcgttgtcggcggcaatcagcctcgtcagatcgacgctacgtaactagtctagaaa

SEQ ID NO: 240 - DNA, 763 bp, synthetic construct
  source 1..763: mol_type = other DNA
gaattctcgagtaatacgactcactatagcgtcgatcgacgaggctgattgccgccgaaacgagatggagctcatcgacacagcatcttcccagacgtgatgccgacgacctagcagctgagaggacgggaatgggaacattttggcggactggacacaatcgcctgcacgggaatctattgattctcttcctgtcggactgaggagcttcgcctcctgacctttgattgctgccatttggcattcttcccagatgcccttgacagaatcaacctgaagaagaacaagctgcaaagacgctcattgaagagacgcgcgtcggcatccggtcagtggcagtgaagggccaacagttcctgattaaccacaaaccgttctactttactggctttggtcgtcatgaagatgcggacttacgtggcaaaggattcgataacgtgctgacgtcgtcttcaatgagcgtctttgcaagcttgttcttcttcaggttgagttctgtcaagggcatctgggacagaatgccaaatggcagcaattcaaaggtcaggaggcgaagctgcctcagtccgacaggaagagagatcaatagattcccgtgcaggtcgattgtgtccagtccgccaaaaatgttcccattcccgtcctcctcagctgctaggtcgtcggcagtcacgtctgggaagatgctgtcgtcgatgagctccatctcgttgtcggcggcaatcagcctcgtcagatcgacgctacgtaactagtctagaaa

SEQ ID NO: 241 - DNA, 904 bp, synthetic construct
  source 1..904: mol_type = other DNA
gaattctcgagtaatacgactcactatagggaatctattgatctctcttcctgtcggactgaggcagcttcgcctcctgacctttgaattgctgccatttggcattctgtcccagatgcccttgacagaactcaacctgaagaagaacaagcttgcaaagacgctcattgaagacgaccgcgtcggcatccggtcagtggcagtgaagggccaacagttcctgattaaccacaaaccgttctactttactggctttggtcgtcatgaagatgcggacttacgtggcaaaggattcgataacgtgctgagtcgtcttcaatgagcgtctttgcaagcttgttcttcttcaggttgagttctgtcaagggcatctgggacagaatgccaaatggcagcaattcaaaggtcaggaggcgaagctgcctcagtccgacaggaagagagatcaatagattcccgtacaggtcgattgtgtccagtccgccaaaaatgttcccattcccgtcctcctcagctgctaggtcgtcggcagtcacgtctgggaagatgctgtcgtcgatgagctccatctcgttgtcggcggcaatcagcctcgtcagatcgacgcttggtgcacgaccacgcattaatggactggattggggccaactcctaccgtacctcgcattacccttacgctgaagagatgctcgactgggcagatgaacatggcatcgtggtgattgatgaaactgctgcagcgtcgatctgacgaggctgattgccgccgacaacgagatggagctcatcgacgacagcatcttcccagacgtgactgccgacgacctagcagctgaggaggacgggaatgggaacatttttggcggactggacacaatcgacctgtacgtaactagtctagaaa

SEQ ID NO: 242 - DNA, 777 bp, synthetic construct
  source 1..777: mol_type = other DNA
gaattctcgagtaatacgactcactataggaaaaccagcggacagagcgttgttcgtaatgaggacttcagccccatcgtgctcacgaagcaggtcggcaatgtcggccaggttgactttgcacctgtcgaagtaccggccgatcagctaaagaatcgtttggaaaaactgcgcatcaaccagatgctgctggactcgtacgccgagggagagctcgacttcagcgacctgcaagaggaaggcatctcgggaggtcccgaagacactgacgaatttgcgcgacagggtggtttcttgaacactcggcgtggcggccgatctgctgaggcgcgtcggcatccggtcagtggcagtgaagggccaacagttcctgattaaccacaaaccgttctactttactggctttggtcgtcatgaagatgcggacttacgtggcaaaggattcgataacgtgctgacctcagcagatcggccgccacgccgagtgttcaagaaaccaccctgtcgcgcaaattcgtcagtgtcttcgggacctcccgagatgccttcctcttgcaggtcgctgaagtcgagctctccctcggcgtacgagtccagcagcatctggttgatgcgcagtttttccaaacgattctttagctgatcggccggtacttcgacaggtgcaaagtcaacctggccgacattgccgacctgcttcgtgagcacgatggggctgaagtcctcattacgaacaacgctctgtccgctggttttcctacgtaactagtctagaaa

SEQ ID NO: 243 - DNA, 777 bp, synthetic construct
  source 1..777: mol_type = other DNA
gaattctcgagtaatacgactcactataggaaaattagtggatagagtgttgtttgtaatgaggattttagttttattgtgtttatgaagtaggttggtaatgttggttaggttgattttgtatttgttgaagtattggttgattagttaaagaattgtttggaaaaattgtgtattaattagatgttgttggatttgtatgttgagggagagtttgattttagtgatttgtaagaggaaggtattttgggaggttttgaagatattgatgaatttgtgtgatagggtggttttttgaatatttggtgtggtggttgatttgttgaggcgcgtcggcatccggtcagtggcagtgaagggccaacagttcctgattaaccacaaaccgttctactttactggctttggtcgtcatgaagatgcggacttacgtggcaaaggattcgataacgtgctgacctcagcagatcggccgccacgccgagtgttcaagaaaccaccctgtcgcgcaaattcgtcagtgtcttcgggacctcccgagatgccttcctcttgcaggtcgctgaagtcgagctctccctcggcgtacgagtccagcagcatctggttgatgcgcagtttttccaaacgattctttagctgatcggccggtacttcgacaggtgcaaagtcaacctggccgacattgccgacctgcttcgtgagcacgatggggctgaagtcctcattacgaacaacgctctgtccgctggttttcctacgtaactagtctagaaa

SEQ ID NO: 244 - DNA, 763 bp, synthetic construct
  source 1..763: mol_type = other DNA
gaattctcgagtaatacgactcactataggaaaaccagggacagagcgttgttcgtaataggacttcagccccatcgtgccacgaagcaggtcggcaatgtggccaggttgactttgcaccttcgaagtaccggccgatcagcaaagaatcgtttggaaaaactcgcatcaaccagatgctgctgactcgtacgccgagggagagccgacttcagcgacctgcaagagaaggcatctcgggaggtcccaagacactgacgaatttgcgcacagggtggtttcttgaacaccggcgtggcggccgatctgctaggcgcgtcggcatccggtcagtggcagtgaagggccaacagttcctgattaaccacaaaccgttctactttactggctttggtcgtcatgaagatgcggacttacgtggcaaaggattcgataacgtgctgacctcagcagatcggccgccacgccgagtgttcaagaaaccaccctgtcgcgcaaattcgtcagtgtcttcgggacctcccgagatgccttcctcttgcaggtcgctgaagtcgagctctccctcggcgtacgagtccagcagcatctggttgatgcgcagtttttccaaacgattctttagctgatcggccggtacttcgacaggtgcaaagtcaacctggccgacattgccgacctgcttcgtgagcacgatggggctgaagtcctcattacgaacaacgctctgtccgctggttttcctacgtaactagtctagaaa

SEQ ID NO: 245 - DNA, 777 bp, synthetic construct
  source 1..777: mol_type = other DNA
gaattctcgagtaatacgactcactatagggatcggggctataagtctgtgatcttcgaagctggtaacgacaccggcggcacatggcgatggaactgctaccctggcgccggcgttgactcagaggtgccagagtacgagttttcgtggcctgaggtctggaaggactggaactggtcgagcaactaccctaggtacgacgaacttcgagcctattttgaccatgtcgacaaggtcctcggcatcaagaaagactgcgccttcaacacggttgttgtgggtgcacactttgacactgccaccggtcgttggaacgttcgcacggcggcgcgtcggcatccggtcagtggcagtgaagggccaacagttcctgattaaccacaaaccgttctactttactggctttggtcgtcatgaagatgcggacttacgtggcaaaggattcgataacgtgctgaccgccgtgcgaacgttccaacgaccggtggcagtgtcaaagtgtgcacccacaacaaccgtgttgaaggcgcagtctttcttgatgccgaggaccttgtcgacatggtcaaaataggctcgaagttcgtcgtacctagggtagttgctcgaccagttccagtccttccagacctcaggccacgaaaactcgtactctggcacctctgagtcaacgccggcgccagggtagcagttccatcgccatgtgccgccggtgtcgttaccagcttcgaagatcacagacttatagccccgatccctacgtaactagtctagaaa

SEQ ID NO: 246 - DNA, 777 bp, synthetic construct
  source 1..777: mol_type = other DNA
gaattctcgagtaatacgactcactatagggattggggttataagtttgtgatctttgaagttggtaatgatattggtggtatatggtgatggaattgttattttggtgttggtgttgatttagaggtgttagagtatgagttttcgtggtttgaggtttggaaggattggaattggttgagtaattattttaggtatgatgaattttgagtttattttgattatgttgataaggttcttggtattaagaaagattgtgtctttaatatggttgttgtgggtgtatattttgatattgttattggttgttggaatgtttgtatggtggcgcgtcggcatccggtcagtggcagtgaagggccaacagttcctgattaaccacaaaccgttctactttactggctttggtcgtcatgaagatgcggacttacgtggcaaaggattcgataacgtgctgaccgccgtgcgaacgttccaacgaccggtggcagtgtcaaagtgtgcacccacaacaaccgtgttgaaggcgcagtctttcttgatgccgaggaccttgtcgacatggtcaaaataggctcgaagttcgtcgtacctagggtagttgctcgaccagttccagtccttccagacctcaggccacgaaaactcgtactctggcacctctgagtcaacgccggcgccagggtagcagttccatcgccatgtgccgccggtgtcgttaccagcttcgaagatcacagacttatagccccgatccctacgtaactagtctagaaa

SEQ ID NO: 247 - DNA, 763 bp, synthetic construct
  source 1..763: mol_type = other DNA
gaattctcgagtaatacgactcactatagggatcggggtataagtctgtgatcttcgaactggtaacgacaccggcggcaatggcgatggaactgctacccggcgccggcgttgactcagagtgccagagtacgagttttcgtgcctgaggtctggaaggactgaactggtcgagcaactaccctggtacgacgaacttcgagcctttttgaccatgtcgacaaggtctcggcatcaagaaagactgcccttcaacacggttgttgtggtgcacactttgacactgccacggtcgttggaacgttcgcacgcggcgcgtcggcatccggtcagtggcagtgaagggccaacagttcctgattaaccacaaaccgttctactttactggctttggtcgtcatgaagatgcggacttacgtggcaaaggattcgataacgtgctgaccgccgtgcgaacgttccaacgaccggtggcagtgtcaaagtgtgcacccacaacaaccgtgttgaaggcgcagtctttcttgatgccgaggaccttgtcgacatggtcaaaataggctcgaagttcgtcgtacctagggtagttgctcgaccagttccagtccttccagacctcaggccacgaaaactcgtactctggcacctctgagtcaacgccggcgccagggtagcagttccatcgccatgtgccgccggtgtcgttaccagcttcgaagatcacagacttatagccccgatccctacgtaactagtctagaaa

SEQ ID NO: 248 - DNA, 777 bp, synthetic construct
  source 1..777: mol_type = other DNA
gaattctcgagtaatacgactcactatagccgtatcgatttggcagtaccacccctttaccctaacgagtgcgcctgaggaagattacatctctatccacatgcgcgtagttggtgatttcactcgtgctgtctctaccgtattgggctgcgagttcgacaagaagaaggacggtagcaaggtcgtcggtgtcaatggcgatggaagcgatgtcgatcctgctttgcgccgcgtgctgcctcgcgtttacatcgacggaccctttggatcggcttcggaggatgttttcaagtacgagatttctgttctctgcggcgcaggaatcggccgcgtcggcatccggtcagtggcagtgaagggccaacagttcctgattaaccacaaaccgttctactttactggctttggtcgtcatgaagatgcggacttacgtggcaaaggattcgataacgtgctgagccgattcctgcgccgcagagaacagaaatctcgtacttgaaaacatcctccgaagccgatccaaagggtccgtcgatgtaaacgcgaggcagcacgcggcgcaaagcaggatcgacatcgcttccatcgccattgacaccgacgaccttgctaccgtccttcttcttgtcgaactcgcagcccaatacggtagagacagcacgagtgaaatcaccaactacgcgcatgtggatagagatgtaatcttcctcaggcgcactcgttagggtaaaggggtggtactgccaaatcgatacggctacgtaactagtctagaaa

SEQ ID NO: 249 - DNA, 777 bp, synthetic construct
  source 1..777: mol_type = other DNA
gaattctcgagtaatacgactcactatagttgtattgatttggtagtattatttctttattttaatgagtgtgtttgaggaagattatatttttatttatatgtgtgtagttggtgattttatttgtgttgtttttattgtattgggttgtgagtttgataagaagaaggatggtagtaaggttgttggtgttaatggtgatggaagtgatgttgattttgttttgtgttgtgtgttgttttgtgtttatattgatggattctttggattggttttggaggatgtttttaagtatgagatttctgttctttgtggtgtaggaattggtcgcgtcggcatccggtcagtggcagtgaagggccaacagttcctgattaaccacaaaccgttctactttactggctttggtcgtcatgaagatgcggacttacgtggcaaaggattcgataacgtgctgagccgattcctgcgccgcagagaacagaaatctcgtacttgaaaacatcctccgaagccgatccaaagggtccgtcgatgtaaacgcgaggcagcacgcggcgcaaagcaggatcgacatcgcttccatcgccattgacaccgacgaccttgctaccgtccttcttcttgtcgaactcgcagcccaatacggtagagacagcacgagtgaaatcaccaactacgcgcatgtggatagagatgtaatcttcctcaggcgcactcgttagggtaaaggggtggtactgccaaatcgatacggctacgtaactagtctagaaa

SEQ ID NO: 250 - DNA, 763 bp, synthetic construct
  source 1..763: mol_type = other DNA
gaattctcgagtaatacgactcactatagccgtatcgattggcagtaccacccctttacctaacgagtgcgcctgaggaaattacatctctatccacatgccgtagttggtgatttcactcggctgtctctaccgtattgggcgcgagttcgacaagaagaaggcggtagcaaggtcgtcggtgtaatggcgatggaagcgatgtcatcctgctttgcgccgcgtgcgcctcgcgtttacatcgacggccctttggatcggcttcggagatgttttcaagtacgagattttgttctctgcggcgcaggaatggccgcgtcggcatccggtcagtggcagtgaagggccaacagttcctgattaaccacaaaccgttctactttactggctttggtcgtcatgaagatgcggacttacgtggcaaaggattcgataacgtgctgagccgattcctgcgccgcagagaacagaaatctcgtacttgaaaacatcctccgaagccgatccaaagggtccgtcgatgtaaacgcgaggcagcacgcggcgcaaagcaggatcgacatcgcttccatcgccattgacaccgacgaccttgctaccgtccttcttcttgtcgaactcgcagcccaatacggtagagacagcacgagtgaaatcaccaactacgcgcatgtggatagagatgtaatcttcctcaggcgcactcgttagggtaaaggggtggtactgccaaatcgatacggctacgtaactagtctagaaa

SEQ ID NO: 251 - DNA, 777 bp, synthetic construct
  source 1..777: mol_type = other DNA
gaattctcgagtaatacgactcactatagattggcctcatggttatcgctcgcatactcatggttgccctcatcattggcaatggatggattggcggagaatggcacattggcttcgccgttcaccagcgcatgatcctcggcatgcgcggggcttacattacgcaagccatgcgaatcatgctgttcatcgtatggtacgggtcgcaggcatggctcggtggtctttgcgtctcagctatgatcagcagctggagctcaagctttctgaacatggagaacacatttgcagcgagcgccaacatggtcactcgtgactttatcgggttcgcgtcggcatccggtcagtggcagtgaagggccaacagttcctgattaaccacaaaccgttctactttactggctttggtcgtcatgaagatgcggacttacgtggcaaaggattcgataacgtgctgaaacccgataaagtcacgagtgaccatgttggcgctcgctgcaaatgtgttctccatgttcagaaagcttgagctccagctgctgatcatagctgagacgcaaagaccaccgagccatgcctgcgacccgtaccatacgatgaacagcatgattcgcatggcttgcgtaatgtaagccccgcgcatgccgaggatcatgcgctggtgaacggcgaagccaatgtgccattctccgccaatccatccattgccaatgatgagggcaaccatgagtatgcgagcgataaccatgaggccaatctacgtaactagtctagaaa

SEQ ID NO: 252 - DNA, 777 bp, synthetic construct
  source 1..777: mol_type = other DNA
gaattctcgagtaatacgactcactatagattggttttatggttattgtttgtatatttatggttgttcttattattggtaatggatggattggtggagaatggtatattggttttgttgtttattagtgtatgattcttggtatgtgtggggtttatattatgtaagttatgtgaattatgttgtttattgtatggtatgggttgtaggtatggtttggtggtctttgtgttttagttatgattagtagttggagtttaagttttctgaatatggagaatatatttgtagtgagtgttaatatggttatttgtgattttattgggttcgcgtcggcatccggtcagtggcagtgaagggccaacagttcctgattaaccacaaaccgttctactttactggctttggtcgtcatgaagatgcggacttacgtggcaaaggattcgataacgtgctgaaacccgataaagtcacgagtgaccatgttggcgctcgctgcaaatgtgttctccatgttcagaaagcttgagctccagctgctgatcatagctgagacgcaaagaccaccgagccatgcctgcgacccgtaccatacgatgaacagcatgattcgcatggcttgcgtaatgtaagccccgcgcatgccgaggatcatgcgctggtgaacggcgaagccaatgtgccattctccgccaatccatccattgccaatgatgagggcaaccatgagtatgcgagcgataaccatgaggccaatctacgtaactagtctagaaa

SEQ ID NO: 253 - DNA, 763 bp, synthetic construct
  source 1..763: mol_type = other DNA
gaattctcgagtaatacgactcactatagattggcctctggttatcgctcgcatactcaggttgccctcatcattggcaaggatggattggcggagaatggacattggcttcgccgttcaccgcgcatgatcctcggcatgcgggggcttacattacgcaagcctgcgaatcatgctgttcatcgatggtacgggtcgcaggcatgctcggtggtctttgcgtctcactatgatcagcagctggagctaagctttctgaacatggagaaacatttgcagcgagcgccaactggtcactcgtgactttatcggttcgcgtcggcatccggtcagtggcagtgaagggccaacagttcctgattaaccacaaaccgttctactttactggctttggtcgtcatgaagatgcggacttacgtggcaaaggattcgataacgtgctgaaacccgataaagtcacgagtgaccatgttggcgctcgctgcaaatgtgttctccatgttcagaaagcttgagctccagctgctgatcatagctgagacgcaaagaccaccgagccatgcctgcgacccgtaccatacgatgaacagcatgattcgcatggcttgcgtaatgtaagccccgcgcatgccgaggatcatgcgctggtgaacggcgaagccaatgtgccattctccgccaatccatccattgccaatgatgagggcaaccatgagtatgcgagcgataaccatgaggccaatctacgtaactagtctagaaa

SEQ ID NO: 254 - DNA, 777 bp, synthetic construct
  source 1..777: mol_type = other DNA
gaattctcgagtaatacgactcactataggcaacggcattaccaagcctgctgtggcccccaagaccaacagcaactgggatgcctttgccttcgagccgatccgcgagtcgcaggtcgctcgcaccatgggccgccgctacttcgaggacctcgacacctacgccgagtctgacgtcgtcattgtcggtgctggcagctgcggtctctccgccgcctactcgctcgccaatcagcgccccgacctgaagattgccattatcgaggctggcgtcgctcccggcggcggtgcttggctcggcggtcagctcttcagcgccatggtcatgcgcgtcggcatccggtcagtggcagtgaagggccaacagttcctgattaaccacaaaccgttctactttactggctttggtcgtcatgaagatgcggacttacgtggcaaaggattcgataacgtgctgacatgaccatggcgctgaagagctgaccgccgagccaagcaccgccgccgggagcgacgccagcctcgataatggcaatcttcaggtcggggcgctgattggcgagcgagtaggcggcggagagaccgcagctgccagcaccgacaatgacgacgtcagactcggcgtaggtgtcgaggtcctcgaagtagcggcggcccatggtgcgagcgacctgcgactcgcggatcggctcgaaggcaaaggcatcccagttgctgttggtcttgggggccacagcaggcttggtaatgccgttgcctacgtaactagtctagaaa

SEQ ID NO: 255 - DNA, 777 bp, synthetic construct
  source 1..777: mol_type = other DNA
gaattctcgagtaatacgactcactataggtaatggtattattaagtttgttgtggtttttaagattaatagtaattgggatgtctttgtctttgagttgatttgtgagttgtaggttgtttgtattatgggttgttgttattttgaggattttgatatttatgttgagtttgatgttgttattgttggtgttggtagttgtggtttttctgttgtttatttgtttgttaattagtgttttgatttgaagattgttattattgaggttggtgttgtttttggtggtggtgtttggtttggtggttagttctttagtgttatggttatgcgcgtcggcatccggtcagtggcagtgaagggccaacagttcctgattaaccacaaaccgttctactttactggctttggtcgtcatgaagatgcggacttacgtggcaaaggattcgataacgtgctgacatgaccatggcgctgaagagctgaccgccgagccaagcaccgccgccgggagcgacgccagcctcgataatggcaatcttcaggtcggggcgctgattggcgagcgagtaggcggcggagagaccgcagctgccagcaccgacaatgacgacgtcagactcggcgtaggtgtcgaggtcctcgaagtagcggcggcccatggtgcgagcgacctgcgactcgcggatcggctcgaaggcaaaggcatcccagttgctgttggtcttgggggccacagcaggcttggtaatgccgttgcctacgtaactagtctagaaa

SEQ ID NO: 256 - DNA, 763 bp, synthetic construct
  source 1..763: mol_type = other DNA
gaattctcgagtaatacgactcactataggcaacggcataccaagcctgctgtggccccaagaccaacagcaactgggatcctttgccttcgagccgatcccgagtcgcaggtcgctcgcacatgggccgccgctacttcgagacctcgacacctacgccgagttgacgtcgtcattgtcggtgcggcagctgcggtctctccgcccctactcgctcgccaatcagcccccgacctgaagattgccatatcgaggctggcgtcgctcccgcggcggtgcttggctcggcgtcagctcttcagcgccatggtatgcgcgtcggcatccggtcagtggcagtgaagggccaacagttcctgattaaccacaaaccgttctactttactggctttggtcgtcatgaagatgcggacttacgtggcaaaggattcgataacgtgctgacatgaccatggcgctgaagagctgaccgccgagccaagcaccgccgccgggagcgacgccagcctcgataatggcaatcttcaggtcggggcgctgattggcgagcgagtaggcggcggagagaccgcagctgccagcaccgacaatgacgacgtcagactcggcgtaggtgtcgaggtcctcgaagtagcggcggcccatggtgcgagcgacctgcgactcgcggatcggctcgaaggcaaaggcatcccagttgctgttggtcttgggggccacagcaggcttggtaatgccgttgcctacgtaactagtctagaaa

SEQ ID NO: 257 - DNA, 710 bp, synthetic construct
  source 1..710: mol_type = other DNA
accagaaacccggcatgcccagatgttagtcatttttgatcttttggaatcaaaaacgttttattttgtgttcggatacgagtctcattcattcgtttctcatccatgtcagcagcagctgaatttacatatcctggccttccattgtctgacactttaatcagtgtaaggacatctttgcgctgccttcaattgtgctatattaacactcgtcgaaaacttctgaacagcttatttaggctctccagatatctgcagtccactcgactcacaacgaaggagtttctcataaccttctttgcgatttgtcggtcacttgtacttcaaaaatgacaagaaacatgtttttgatgcatctcgcagtaatttgcctcctgtgcatcgcaagaagcgaatcacggcgctgcacgaacggacgaacagcgtcatgccaagaacataatacactacacaagcagatgtatcccgatgtcaacgacgaaaagtgccagggtttttggacatgttataacgggacagccgtactacgatgctgtggtgtgggaacgagatacagttccgaactccaggtctgcgtgcacgccaaagacgtgcactgcccaaaatctgaccatgcttctttgcatatacaaccaagattaaatagatccaaagaacgaaagtaaataaagatatgaacatattaaaaaataaaaaagtcatgcatgctt

SEQ ID NO: 258 - DNA, 1181 bp, synthetic construct
  source 1..1181: mol_type = other DNA
atgcagaaatttcagctcgtatcaatgctttgtgcatcgttcctgattggcttaacgataagtcaaggcgctcaagcacgacttctacacaaacgctcgttcctccaaatgggctgtaaaggtgaatttgtcccatccttcttcgcacgtctggatcgtgtttgtgaggaatgcttccagatgtaccgcaaaccatttattttcgactcatgccgatcggattgttaccggaatgagacatttttgttgtgcactgaagccctaatgctccacaaggaaaaggatatgcttgaaagtatggtaaatcaactctacggacctatcccttaaatcagcaggagaccaactgcgtcacaaccgctacgagtcttcagctaaacaaatcggttacagcaaaccaagagaatattaactggacaaaatttcttttagcatttaatacgaaaaaaaaccgtaacttgaacgcttcactcaaataaattcaccatctagaaaatagaaataatacctacgtttacctttaaatgaaatttaggaaaaaactaggttgggttgaagcaaaggcaacgctgtttatttatgcacttctgcgaagaagtggttttattaaagttagcttaacatttgatggtaagattcgcatctcttggaaagaggcacgagagcgtcgtttaccaatcccgactatgacaactttaaaaaacgacccgccgagcatgacgactaaagctgttcgtatcgtaaatcacaactgtcagtttcttcaagaatacatttatcgatgttacgcttatgcctgtcaggccacttatgatacattttaactgtgactatcatccattggtaagaattcagtgttgatggaatgttttatctttaaaggaccaactaatcagaggaaataaaaataaacaaatatactatttgctgttttgtaaagtaggatattagaagaacaacagaagtgtttaatttttggttgcctttttatcaaatatatacatttacgcgaatgcccctctttgccgggccttagacccaaccaaacgagtatccatatctgtgagcgtgtggagaatttacaaactaatcaaaaagcgcgtgacgttaactttaaaaacgaatggtatataaaaatgctggtttctatgttcaacttctacgacagaataaaagcaata

SEQ ID NO: 259 - DNA, 1498 bp, synthetic construct
  source 1..1498: mol_type = other DNA
ctaccattgtttagcccattttcgcgctgtgtctgtcggcgactgcggaagtactgagctagctgagtcgtggtgcttagaaggcagtggcagtgagtggcatcagcggtaataagtgagggacaacagccggagagtcgtgtcggttggtaggtcggttcgttgccgattaatgccccacatgtgagttacgtgcggtgcttgttccgctgtgcattggacgttacatacagagagtcaggtgtagtttattttagacgaaaaactaccagcactacctgatacagcgacccaacgtagagagggaaagagaaagagctgttgtttcgctaggttagttctgaacaattggattgattcgcaaatgtacgcttgtacggctaacggttgaacggactgtgcaaactgcggatagtgcgtgaactagcagacaacatatccaataactaactacacggttattattgatacaaacacctagttcagacagacaatttcggtttgctttgttggcactataaggagtaagtgatattgttttttgtttatcagtcggcctagtattgtcggtgcacttacggataacaggagaggcagtacaaaggcaactcgattcactatcgttcacgggcgctaatacagcgaaatattaataggcaaagcaagcaagccagccggccaagcccaaaccccgggcgaaacgcagataccaacagcgctgaagtgcgtggcaaacgacaacgggacagtagggcataagctagacagcatatagcttttctaaccatggctgatcagctaactgaggaacagatcgccgagttcaaagaggcgtttagcctgtttgacaaggacggagatggcacgatcacgacaaaggagctcggtacggtaatgcgatctctcggccagaaccccactgaggctgaactgcaggacatgatcaacgaggtcgacgccgacggctccggaacgatagatttccctgagttcctcacaatgatggcaagaaagatgaaggacaccgactcggaggaggagatccgagaggcgttccgcgtattcgacaaggatggcaacggtttcatttcggcggccgagctcaggcacgttatgaccaaccttggcgagaagcttacggacgaggaggtagatgagatgattcgggaggcagatattgacggtgatggtcaggtcaactacgaggagttcgtcaccatgatgacgtccaagtaaatatatgataatgttggctgtctcgtgtaatgtcgtgagaaagaaagcgcgcgcgaaagaaagagagaaaggaatagaaaactataaatagcttgttgttaaagcaacgcaacaacaagctgtaagcaacaaacaatatttacgaagtatacgataatgaaagtcgacagggaagcaagcacggatatatatgaaaactaagcgaaatgacgtcgtcatcatcaccagcagcagcagcagcagcagcagcagcagcagcagc

SEQ ID NO: 260 - protein, 111 aa, Varroa destructor
MTRNMFLMHLAVICLLCIARSESRRCTNGRTASCQEHNTLHKQMYPDVNDEKCQGFWTCYNGTAVLRCCGVGTRYSSELQVCVHAKDVHCPKSDHASLHIQPRLNRSKERK

SEQ ID NO: 261 - protein, 109 aa, Varroa destructor
MQKFQLVSMLCASFLIGLTISQGAQARLLHKRSFLQMGCKGEFVPSFFARLDRVCEECFQMYRKPFIFDSCRSDCYRNETFLLCTEALMLHKEKDMLESMVNQLYGPIP

SEQ ID NO: 262 - protein, 149 aa, Varroa destructor
MADQLTEEQIAEFKEAFSLFDKDGDGTITTKELGTVMRSLGQNPTEAELQDMINEVDADGSGTIDFPEFLTMMARKMKDTDSEEEIREAFRVFDKDGNGFISAAELRHVMTNLGEKLTDEEVDEMIREADIDGDGQVNYEEFVTMMTSK

SEQ ID NO: 263 - DNA, 500 bp, synthetic construct
  source 1..500: mol_type = other DNA
gtaaggacatctttgcgctgccttcaattgtgctatattaacactcgtcgaaaacttctgaacagcttatttaggctctccagatatctgcagtccactcgactcacaacgaaggagtttctcataaccttctttgcgatttgtcggtcacttgtacttcaaaaatgacaagaaacatgtttttgatgcatctcgcagtaatttgcctcctgtgcatcgcaagaagcgaatcacggcgctgcacgaacggacgaacagcgtcatgccaagaacataatacactacacaagcagatgtatcccgatgtcaacgacgaaaagtgccagggtttttggacatgttataacgggacagccgtactacgatgctgtggtgtgggaacgagatacagttccgaactccaggtctgcgtgcacgccaaagacgtgcactgcccaaaatctgaccatgcttctttgcatatacaaccaagattaaatagatccaaagaacgaaagtaa

SEQ ID NO: 264 - DNA, 500 bp, synthetic construct
  source 1..500: mol_type = other DNA
gggctgtaaaggtgaatttgtcccatccttcttcgcacgtctggatcgtgtttgtgaggaatgcttccagatgtaccgcaaaccatttattttcgactcatgccgatcggattgttaccggaatgagacatttttgttgtgcactgaagccctaatgctccacaaggaaaaggatatgcttgaaagtatggtaaatcaactctacggacctatcccttaaatcagcaggagaccaactgcgtcacaaccgctacgagtcttcagctaaacaaatcggttacagcaaaccaagagaatattaactggacaaaatttcttttagcatttaatacgaaaaaaaaccgtaacttgaacgcttcactcaaataaattcaccatctagaaaatagaaataatacctacgtttacctttaaatgaaatttaggaaaaaactaggttgggttgaagcaaaggcaacgctgtttatttatgcacttctgcgaagaagtggttttattaa

SEQ ID NO: 265 - DNA, 500 bp, synthetic construct
  source 1..500: mol_type = other DNA
ctgaggaacagatcgccgagttcaaagaggcgtttagcctgtttgacaaggacggagatggcacgatcacgacaaaggagctcggtacggtaatgcgatctctcggccagaaccccactgaggctgaactgcaggacatgatcaacgaggtcgacgccgacggctccggaacgatagatttccctgagttcctcacaatgatggcaagaaagatgaaggacaccgactcggaggaggagatccgagaggcgttccgcgtattcgacaaggatggcaacggtttcatttcggcggccgagctcaggcacgttatgaccaaccttggcgagaagcttacggacgaggaggtagatgagatgattcgggaggcagatattgacggtgatggtcaggtcaactacgaggagttcgtcaccatgatgacgtccaagtaaatatatgataatgttggctgtctcgtgtaatgtcgtgagaaagaaagcgcgcgcgaaagaaagaga

SEQ ID NO: 266 - DNA, 1337 bp, synthetic construct
  source 1..1337: mol_type = other DNA
gaattctaatacgactcactatagcgttcgtgcagcgccgtgattcgcttcttgcgatgcacaggaggcaaattactgcgagatgcatcaaaaacatgtttcttgtcatttttgaagtacaagtgaccgacaaatcgcaaagaaggttatgagaaactccttcgttgtgagtcgagtggactgcagatatctggagagcctaaataagctgttcagaagttttcgacgagtgttaatatagcacaattgaaggcagcgcaaagatgtccttacaatggaccagtaaagagcagtcacatatctattccatagacttgcttctttgaaatttggatacacagctccaatccatgtctttcttggattaggatatctatctgcaatcaggtagttgaaacatccagcacaatgtatagcgaacaagtaaggacatctttgcgctgccttcaattgtgctatattaacactcgtcgaaaacttctgaacagcttatttaggctctccagatatctgcagtccactcgactcacaacgaaggagtttctcataaccttctttgcgatttgtcggtcacttgtacttcaaaaatgacaagaaacatgtttttgatgcatctcgcagtaatttgcctcctgtgcatcgcaagaagcgaatcacggcgctgcacgaacggacgaacagcgtcatgccaagaacataatacactacacaagcagatgtatcccgatgtcaacgacgaaaagtgccagggtttttggacatgttataacgggacagccgtactacgatgctgtggtgtgggaacgagatacagttccgaactccaggtctgcgtgcacgccaaagacgtgcactgcccaaaatctgaccatgcttctttgcatatacaaccaagattaaatagatccaaagaacgaaagtaaaatggaccagtaaagagcagtcacatatctattccatagacttgcttctttgaaatttggatacacagctccaatccatgtctttcttggattaggatatctatctgcaatcaggtagttgaaacatccagcacaatgtatagcgaacaattactttcgttctttggatctatttaatcttggttgtatatgcaaagaagcatggtcagattttgggcagtgcacgtctttggcgtgcacgcagacctggagttcggaactgtatctcgttcccacaccacagcatcgtagtacggctgtcccgttataacatgtccaaaaaccctggcacttttcgtcgttgacatcgggatacatctgcttgtgtagtgtattatgttcttggcatgacgctgtttacgtaagcttactagt

SEQ ID NO: 267 - DNA, 1337 bp, synthetic construct
  source 1..1337: mol_type = other DNA
gaattctaatacgactcactatagcgttcgtgcagcgccgtgattcgcttcttgcgatgcacaggaggcaaattactgcgagatgcatcaaaaacatgtttcttgtcatttttgaagtacaagtgaccgacaaatcgcaaagaaggttatgagaaactccttcgttgtgagtcgagtggactgcagatatctggagagcctaaataagctgttcagaagttttcgacgagtgttaatatagcacaattgaaggcagcgcaaagatgtccttacaatggaccagtaaagagcagtcacatatctattccatagacttgcttctttgaaatttggatacacagctccaatccatgtctttcttggattaggatatctatctgcaatcaggtagttgaaacatccagcacaatgtatagcgaacaagtaaggatatttttgtgttgtttttaattgtgttatattaatatttgttgaaaatttttgaatagtttatttaggttttttagatatttgtagtttatttgatttataatgaaggagttttttataattttttttgtgatttgttggttatttgtattttaaaaatgataagaaatatgtttttgatgtattttgtagtaatttgttttttgtgtattgtaagaagtgaattatggtgttgtatgaatggatgaatagtgttatgttaagaatataatatattatataagtagatgtattttgatgttaatgatgaaaagtgttagggtttttggatatgttataatgggatagttgtattatgatgttgtggtgtgggaatgagatatagttttgaattttaggtttgtgtgtatgttaaagatgtgtattgtttaaaatttgattatgtttttttgtatatataattaagattaaatagatttaaagaatgaaagtaaaatggaccagtaaagagcagtcacatatctattccatagacttgcttctttgaaatttggatacacagctccaatccatgtctttcttggattaggatatctatctgcaatcaggtagttgaaacatccagcacaatgtatagcgaacaattactttcgttctttggatctatttaatcttggttgtatatgcaaagaagcatggtcagattttgggcagtgcacgtctttggcgtgcacgcagacctggagttcggaactgtatctcgttcccacaccacagcatcgtagtacggctgtcccgttataacatgtccaaaaaccctggcacttttcgtcgttgacatcgggatacatctgcttgtgtagtgtattatgttcttggcatgacgctgtttacgtaagcttactagt

SEQ ID NO: 268 - DNA, 1337 bp, synthetic construct
  source 1..1337: mol_type = other DNA
gaattctaatacgactcactatagggttgtgacgcagttggtctcctgctgatttaagggataggtccgtagagttgatttaccatactttcaagcatatccttttccttgtggagcattagggcttcagtgcacaacaaaaatgtctcattccggtaacaatccgatcggcatgagtcgaaaataaatggtttgcggtacatctggaagcattcctcacaaacacgatccagacgtgcgaagaaggatgggacaaattcacctttacagcccaatggaccagtaaagagcagtcacatatctattccatagacttgcttctttgaaatttggatacacagctccaatccatgtctttcttggattaggatatctatctgcaatcaggtagttgaaacatccagcacaatgtatagcgaacaagggctgtaaaggtgaatttgtcccatccttcttcgcacgtctggatcgtgtttgtgaggaatgcttccagatgtaccgcaaaccatttattttcgactcatgccgatcggattgttaccggaatgagacatttttgttgtgcactgaagccctaatgctccacaaggaaaaggatatgcttgaaagtatggtaaatcaactctacggacctatcccttaaatcagcaggagaccaactgcgtcacaaccgctacgagtcttcagctaaacaaatcggttacagcaaaccaagagaatattaactggacaaaatttcttttagcatttaatacgaaaaaaaaccgtaacttgaacgcttcactcaaataaattcaccatctagaaaatagaaataatacctacgtttacctttaaatgaaatttaggaaaaaactaggttgggttgaagcaaaggcaacgctgtttatttatgcacttctgcgaagaagtggttttattaaaatggaccagtaaagagcagtcacatatctattccatagacttgcttctttgaaatttggatacacagctccaatccatgtctttcttggattaggatatctatctgcaatcaggtagttgaaacatccagcacaatgtatagcgaacaattaataaaaccacttcttcgcagaagtgcataaataaacagcgttgcctttgcttcaacccaacctagttttttcctaaatttcatttaaaggtaaacgtaggtattatttctattttctagatggtgaatttatttgagtgaagcgttcaagttacggttttttttcgtattaaatgctaaaagaaattttgtccagttaatattctcttggtttgctgtaaccgatttgtttagctgaagactcgtacgtaagcttactagt

SEQ ID NO: 269 - DNA, 1337 bp, synthetic construct
  source 1..1337: mol_type = other DNA
gaattctaatacgactcactatagggttgtgacgcagttggtctcctgctgatttaagggataggtccgtagagttgatttaccatactttcaagcatatccttttccttgtggagcattagggcttcagtgcacaacaaaaatgtctcattccggtaacaatccgatcggcatgagtcgaaaataaatggtttgcggtacatctggaagcattcctcacaaacacgatccagacgtgcgaagaaggatgggacaaattcacctttacagcccaatggaccagtaaagagcagtcacatatctattccatagacttgcttctttgaaatttggatacacagctccaatccatgtctttcttggattaggatatctatctgcaatcaggtagttgaaacatccagcacaatgtatagcgaacaagggttgtaaaggtgaatttgttttatttttttttgtatgtttggattgtgtttgtgaggaatgtttttagatgtattgtaaattatttatttttgatttatgttgattggattgttattggaatgagatatttttgttgtgtattgaagttttaatgttttataaggaaaaggatatgtttgaaagtatggtaaattaattttatggatttattttttaaattagtaggagattaattgtgttataattgttatgagtttttagttaaataaattggttatagtaaattaagagaatattaattggataaaattttttttagtatttaatatgaaaaaaaattgtaatttgaatgttttatttaaataaatttattatttagaaaatagaaataatatttatgtttatttttaaatgaaatttaggaaaaaattaggttgggttgaagtaaaggtaatgttgtttatttatgtatttttgtgaagaagtggttttattaaaatggaccagtaaagagcagtcacatatctattccatagacttgcttctttgaaatttggatacacagctccaatccatgtctttcttggattaggatatctatctgcaatcaggtagttgaaacatccagcacaatgtatagcgaacaattaataaaaccacttcttcgcagaagtgcataaataaacagcgttgcctttgcttcaacccaacctagttttttcctaaatttcatttaaaggtaaacgtaggtattatttctattttctagatggtgaatttatttgagtgaagcgttcaagttacggttttttttcgtattaaatgctaaaagaaattttgtccagttaatattctcttggtttgctgtaaccgatttgtttagctgaagactcgtacgtaagcttactagt

SEQ ID NO: 270 - DNA, 1337 bp, synthetic construct
  source 1..1337: mol_type = other DNA
gaattctaatacgactcactatagcctctcggatctcctcctccgagtcggtgtccttcatctttcttgccatcattgtgaggaactcagggaaatctatcgttccggagccgtcggcgtcgacctcgttgatcatgtcctgcagttcagcctcagtggggttctggccgagagatcgcattaccgtaccgagctcctttgtcgtgatcgtgccatctccgtccttgtcaaacaggctaaacgcctctttgaactcggcgatctgttcctcagttagctgatcagccatggttagaaaagctatatgctgtctagcttatgccctactgtcccgttgtcgtttgccacgcacttcagcgctgttggtatctgcgtttcgcccggggtttgggcttggccggctggcttgcttgctttgcctatctgaggaacagatcgccgagttcaaagaggcgtttagcctgtttgacaaggacggagatggcacgatcacgacaaaggagctcggtacggtaatgcgatctctcggccagaaccccactgaggctgaactgcaggacatgatcaacgaggtcgacgccgacggctccggaacgatagatttccctgagttcctcacaatgatggcaagaaagatgaaggacaccgactcggaggaggagatccgagaggcgttccgcgtattcgacaaggatggcaacggtttcatttcggcggccgagctcaggcacgttatgaccaaccttggcgagaagcttacggacgaggaggtagatgagatgattcgggaggcagatattgacggtgatggtcaggtcaactacgaggagttcgtcaccatgatgacgtccaagtaaatatatgataatgttggctgtctcgtgtaatgtcgtgagaaagaaagcgcgcgcgaaagaaagagaacgacgtcatttcgcttagttttcatatatatccgtgcttgcttccctgtcgactttcattatcgtatacttcgtaaatattgtttgttgcttacagcttgttgttgcgttgctttaacaacaagctatttatagttttctattcctttctctctttctttcgcgcgcgctttctttctcacgacattacacgagacagccaacattatcatatatttacttggacgtcatcatggtgacgaactcctcgtagttgacctgaccatcaccgtcaatatctgcctcccgaatcatctcatctacctcctcgtccgtaagcttctcgccaaggttggtcataacgtgcctgagctcggccgccgaaatgaaaccgttgccatccttgtcgaatacgcggtacgtaagcttactagt

SEQ ID NO: 271 - DNA, 1337 bp, synthetic construct
  source 1..1337: mol_type = other DNA
gaattctaatacgactcactatagcctctcggatctcctcctccgagtcggtgtccttcatctttcttgccatcattgtgaggaactcagggaaatctatcgttccggagccgtcggcgtcgacctcgttgatcatgtcctgcagttcagcctcagtggggttctggccgagagatcgcattaccgtaccgagctcctttgtcgtgatcgtgccatctccgtccttgtcaaacaggctaaacgcctctttgaactcggcgatctgttcctcagttagctgatcagccatggttagaaaagctatatgctgtctagcttatgccctactgtcccgttgtcgtttgccacgcacttcagcgctgttggtatctgcgtttcgcccggggtttgggcttggccggctggcttgcttgctttgcctatttgaggaatagattgttgagtttaaagaggtgtttagtttgtttgataaggatggagatggtatgattatgataaaggagtttggtatggtaatgtgattttttggttagaattttattgaggttgaattgtaggatatgattaatgaggttgatgttgatggttttggaatgatagatttttttgagttttttataatgatggtaagaaagatgaaggatattgatttggaggaggagatttgagaggtgttttgtgtatttgataaggatggtaatggttttattttggtggttgagtttaggtatgttatgattaattttggtgagaagtttatggatgaggaggtagatgagatgatttgggaggtagatattgatggtgatggttaggttaattatgaggagtttgttattatgatgatgtttaagtaaatatatgataatgttggttgttttgtgtaatgttgtgagaaagaaagtgtgtgtgaaagaaagagaacgacgtcatttcgcttagttttcatatatatccgtgcttgcttccctgtcgactttcattatcgtatacttcgtaaatattgtttgttgcttacagcttgttgttgcgttgctttaacaacaagctatttatagttttctattcctttctctctttctttcgcgcgcgctttctttctcacgacattacacgagacagccaacattatcatatatttacttggacgtcatcatggtgacgaactcctcgtagttgacctgaccatcaccgtcaatatctgcctcccgaatcatctcatctacctcctcgtccgtaagcttctcgccaaggttggtcataacgtgcctgagctcggccgccgaaatgaaaccgttgccatccttgtcgaatacgcggtacgtaagcttactagt

SEQ ID NO: 272 - DNA, 1314 bp, synthetic construct
  source 1..1314: mol_type = other DNA
gaattctaatacgactcactatagcctctcggatctcctcctccgagtcggtgtccttcatctttcttgccatcattgtgaggaactcagggaaatctatcgttccggagccgtcggcgtcgacctcgttgatcatgtcctgcagttcagcctcagtggggttctggccgagagatcgcattaccgtaccgagctcctttgtcgtgatcgtgccatctccgtccttgtcaaacaggctaaacgcctctttgaactcggcgatctgttcctcagttagctgatcagccatggttagaaaagctatatgctgtctagcttatgccctactgtcccgttgtcgtttgccacgcacttcagcgctgttggtatctgcgtttcgcccggggtttgggcttggccggctggcttgcttgctttgcctatctgaggaaagatcgccgagttcaaagagggtttagcctgtttgacaaggaggagatggcacgatcacgacaaggagctcggtacggtaatgcatctctcggccagaaccccacgaggctgaactgcaggacatgtcaacgaggtcgacgccgacgctccggaacgatagatttcccgagttcctcacaatgatggcagaaagatgaaggacaccgactggaggaggagatccgagagggttccgcgtattcgacaaggaggcaacggtttcatttcggcgccgagctcaggcacgttatgacaaccttggcgagaagcttacgacgaggaggtagatgagatgttcgggaggcagatattgacgtgatggtcaggtcaactacgagagttcgtcaccatgatgacgccaagtaaatatatgataatgtggctgtctcgtgtaatgtcggagaaagaaagcgcgcgcgaagaaagagaacgacgtcatttcgcttagttttcatatatatccgtgcttgcttccctgtcgactttcattatcgtatacttcgtaaatattgtttgttgcttacagcttgttgttgcgttgctttaacaacaagctatttatagttttctattcctttctctctttctttcgcgcgcgctttctttctcacgacattacacgagacagccaacattatcatatatttacttggacgtcatcatggtgacgaactcctcgtagttgacctgaccatcaccgtcaatatctgcctcccgaatcatctcatctacctcctcgtccgtaagcttctcgccaaggttggtcataacgtgcctgagctcggccgccgaaatgaaaccgttgccatccttgtcgaatacgcggtacgtaagcttactagt

SEQ ID NO: 273 - DNA, 1314 bp, synthetic construct
  source 1..1314: mol_type = other DNA
gaattctaatacgactcactatagcctctcggatctcctcctccgagtcggtgtccttcatctttcttgccatcattgtgaggaactcagggaaatctatcgttccggagccgtcggcgtcgacctcgttgatcatgtcctgcagttcagcctcagtggggttctggccgagagatcgcattaccgtaccgagctcctttgtcgtgatcgtgccatctccgtccttgtcaaacaggctaaacgcctctttgaactcggcgatctgttcctcagttagctgatcagccatggttagaaaagctatatgctgtctagcttatgccctactgtcccgttgtcgtttgccacgcacttcagcgctgttggtatctgcgtttcgcccggggtttgggcttggccggctggcttgcttgctttgcctatttgaggaaagattgttgagtttaaagagggtttagtttgtttgataaggaggagatggtatgattatgataaggagtttggtatggtaatgtattttttggttagaattttatgaggttgaattgtaggatatgttaatgaggttgatgttgatgttttggaatgatagattttttgagttttttataatgatggtagaaagatgaaggatattgattggaggaggagatttgagaggtttttgtgtatttgataaggaggtaatggttttattttggtgttgagtttaggtatgttatgataattttggtgagaagtttatgatgaggaggtagatgagatgtttgggaggtagatattgatgtgatggttaggttaattatgagagtttgttattatgatgatgttaagtaaatatatgataatgtggttgttttgtgtaatgttggagaaagaaagtgtgtgtgaagaaagagaacgacgtcatttcgcttagttttcatatatatccgtgcttgcttccctgtcgactttcattatcgtatacttcgtaaatattgtttgttgcttacagcttgttgttgcgttgctttaacaacaagctatttatagttttctattcctttctctctttctttcgcgcgcgctttctttctcacgacattacacgagacagccaacattatcatatatttacttggacgtcatcatggtgacgaactcctcgtagttgacctgaccatcaccgtcaatatctgcctcccgaatcatctcatctacctcctcgtccgtaagcttctcgccaaggttggtcataacgtgcctgagctcggccgccgaaatgaaaccgttgccatccttgtcgaatacgcggtacgtaagcttactagt

SEQ ID NO: 274 - DNA, 1399 bp, synthetic construct
  source 1..1399: mol_type = other DNA
atggcggcgactacgacaactgctgcttcactagcctgtgcccacttcttaaatcagaacaaggcctacccgtttccgtccaaaacctccgtgcaagtctctcaaatcatcgatgggaggaagatgaggagtgccactctcttctccgctgtctccaccgataagaccatcaccaccaccgctcagtctgtttcacccactgcatgtgacagagtgaggaggcatacaatctcggtgttcgtgggtgacgagagcggtatcataaaccggatagccggagtgtttgctagaagaggatacaacatcgagtctctcgctgtggggttgaacgaagacaaagctatgtttaccattgttgttctcgggactgacaaagtcttgcagcaagttgttgaacagcttaacaaactcgtcaacgtcatcaaggttgaagatctctccaaggagccgcatgtggaacgtgagttaatgcttattaaacttaacgctgatccaaccacccgctctgagatcatgtggttggtggatatctttagagcaaaagtggtggatacatcggatcacactctaacaatcgaggtaactggagacccggggaagatggttgcactaacagccaacttggagaagtttggaataaaagaagtcgccagaactggcaagattgctctgagacgggaaaagatgggagaaactgcaccattctggagattttcagcggcgtcatacccacatcttgtaaatcaatcatctcatgagaatgttgcagagaatacaaagcgagcgttgactggtggaaatggcaatgcatcatctgggggtgacgtttatccagtggagccttataatgatttcaaaccggtgcttgatgctcattggggagtggtctacgacgaagattcgaatggtctccggtcccacacgttatctatactagtagccaacgttcctggagttctcaatctaataaccggagctatctctaggagaggttataacatccagagtctagctgttggtcctgctgagaaagagggcttgtctcggatcactacagtgatccctggaactgatgaaaacatcgacaaattggtgaggcagcttcagaagttgattgatcttcatgaggttcaaaatataacccacatgccatttgcggagcgtgaactgatgctcatcaaagtagctgctcatacttctgcaaggagagatgttcttgatatcgctcaagtgttccgagctaaagccattgatgtctccgatcatactatcactctcgaggtggctaggacaggaagagtggtggctaggacaggaagagtggcattggtaagagaatctggagtggattcgacttaccttcgtggatactctcttcctttgtagttcccaagaggtt

SEQ ID NO: 275 - DNA, 1422 bp, synthetic construct
  source 1..1422: mol_type = other DNA
atgccactggcgatctctctcccaacctcatctcctcctcacctcttccctcgccgcagccgcgttcatcaaaatctccgataccgacgcgtctccatacctcgatcctcctccacccaggagaatcagaccggagtcatcatcgtcggcgccggactcgccggcttagccgccgcaactcgcctcgcctccgcccaaatccctttcctccttctagaagcctccgacggcgtcggcggccgcgtccgcacagacaccaccgtcgacggcttccttctcgacagagggtttcagatcttcatcaccgcgtatcccgaagccaagaagcttctcgactacgaggcgctcgatctccggagattctacgccggagctaaggttttctacggcggggagtttcacaccgtggctgatcctctccgtcacttatgggactccgtggcgtctctcgcgaacccgatcgggtccgttgtcgacaaggggctcatcgcgctgacgagagctagggttctggttaaatcagacgaagacattttgaccagcgccgaagaggttccgaccatcgatctcctccggaagatcggtttctccgatgagatcctcgatcggtttttccggcccttcttcggagggattttcttcgacagagacctcgagacgacctcgaagctcttcgatttcgtgttccggtgtctcgctctcggagaaaacacgcttcccgccatggggatcggtgagatccctaaccagttggccgcgaaactacctgttgattccgtcttgatgaacactagggtggattcgatcgagtatccgaacgggtcgggttcgggtccgcctatagtgagactccaagacggcggcgttttgagtgctgagttgggtgtgatactcgccgtcgagctgccccaagtggataagcttcttaacggtatcagagaaccggttaggactaaaccggctcggagcacggtttgcatttacttcgcagctgaaccgaatcagataccggtggaggatccggttctgtttctcaacgggtcgaacaccggtatcatcaacaacatgttcttccctacgaacgttgctcgaacatacgcgccgccggggaaggctctggtttcggtgtcattgatcggttcgttcgaggatagatcagatgatgatttggcagcagaggttatcagagaactctctggttggttcggtgagtctttggttacgtcgtggagacatttgagaacataccggatccaattcgcccaaccgaaccaatgcccacccactgatttggttaagaatcctcgagttgggtcgggtctttacttatccggtgattacatgacttctgctacgttcgacggtgctttggtctctgggagacgagctgtggaagctttgttacgagaaaaagggctagtt

SEQ ID NO: 276 - DNA, 1701 bp, synthetic construct
  source 1..1701: mol_type = other DNA
atggttgtgtttgggaatgtttctgcagcgaatttgccttatcaaaatgggtttttgaaggcaatttcatctggtggttgtgatttaatgggacaccgcagtttcaaaatttcaacttgttcacaagctcttaagacaagaacaaggaggagtgctggtcctttgaaggtagtttgtgtggatataccaaggccagagctagagaacactgtcaacttcttagaagctgcaagtttgtctgcatctttccgtagtgctcctcgtcctgcgaagcctttaaaagttgtaatcgctggtgctgggttggctggattgtcaacagcaaagtacctggctgatgcaggacatatacctctgttgctcgaagcaagagatgttcttggtggaaagatagctgcatggaaggatgaagatggagattggtatgaaaccggtttacatatattttttggtgcatatccgaatgtgcaaaacttatttggagaacttgggatcaatgatcggttgcagtggaaggaacactccatgatattcgccatgccaagtaaacctggagaattcagtagatttgatttcccagatgtcttaccagcacccttaaacggtatatgggctatattgaggaacaacgagatgctgacatggccagagaaaataaagtttgctattggacttcttccggctatggtcggaggtcaggcttatgttgaggctcaagatggtttatcagttgaacaatggatgagaaagcagggagtacctgatcgagtgactgatgaggtgtttattgccatgtcaaaggcgctgaactttataaacccggacgaactttcaatgcaatgcattttgatagctttgaatcgctttcttcaggagaaacatggatcgaagatggccttcttagatggcaatccaccggagaggctttgtacgccaatagtggaacatatccgatcactaggtggagaggtaagactcaactcgaggataaaaaagattgagctcgacgagaatgatgctactgttaagagtttcttactcactgatggaaccactatccaaggagacgcttatgtctttgccactccagtcgatatcttgaagcttcttttgcctgattcctggaaagagataccatacttcaagagattagagaagttagttggtgtgcctgttatcaacgttcatatatggtttgatcgaaaactaaagaacacatatgatcatttactctttagcagaagtaaccttctgagtgtgtatgctgacatgtcgttaacatgtaaggagtattacgatcctaaccggtcaatgctggagctagtatttgcacctgcagaggaatggatatcaaggaccgactctgacatcattgatgcaaccatggaagagctcgagagactcttccctgacgaaatctcggctgaccaaagcaaagctaaaattctcaagtaccatgtcgttaaaactccaaggtcagtgtacaagaccatcccagactgtgaaccatgtcgtccactacaaagatctcctattaaaggattctacttagctggggattacactaaacagaagtacttagcttccatggaaggtgccgttctctctggcaaattctgctcacagtctattctacaggattacgagatgttggctgcgtctggaccgcaaaagctgtcggaggcgactgtatcaacataa

SEQ ID NO: 277 - DNA, 1359 bp, synthetic construct
  source 1..1359: mol_type = other DNA
atggcgcaagctagcaggatctgccatgtcccatgtcttaatatctccaatctctccaaatcaaaccaaagcaaatcacccttctccctctcgctgaagacaacgcagcagcagcctcctcgagcttcctcgtggggattgaagaagagtgtgatgacgttaactcgtcccaccgtcaaggtaacagcttcctccgtctccacggcggagaaagcagcgtcggagattgtgcttcaacccatcagagaaatctcgggtcttatcaagctacccggatccaaatctctctccaatcggatccttcttctcgccgctctatctgagggaactactgtagtggacaacttgttgaacagtgatgacatcaactacatgctcgatgcgttgaagaagctggggcttaacgtggagcgtgacagtgcaaacaaccgtgctgttgtcgaaggatgcggcgggatcttcccagcttccttggatgccaagggtgatatcgagctgtaccttgggaatgcaggaacagccatgcgtccacttaccgctgctgttactgctgcaggtggcaacgcaagttatgtgcttgatggggtgcctaggatgagggaaagacctataggagatttggttgttggtcttaaacagcttggtgctgatgttgagtgtactctgggaactaactgccctcctgttcgtgtcaatgctaacggtggccttcccggtggaaaggtgaagctttctggatcaatcagtagtcagtacttgactgcactgctcatggcagctccgttagctctcggagacgtggagattgagatcgttgataaactgatctctgttccttacgttgagatgacgttgaagttgatggagcgttacggtgttagtgctgagcatagtgatagctgggatcgtttctttgtcaagggcggtcagaaatacaagtcgcctggtaatgcttatgtagaaggtgatgcttcgagtgctagctacttcttggctggtgctgccattaccggtgaaactgtcactgttgaaggctgtggaacaactagtctgcagggagatgtgaaattcgcagaggttcttgagaaaatgggatgtaaagtgtcgtggacagagaacagtgtgactgtgactggaccatcaagagatgcttttggaatgagacacttgcgtgctgttgacgtcaacatgaacaaaatgcctgatgtagccatgactcttgccgttgttgctctctttgctgatggtccaaccaccattagagatgtggctagctggagagttaaggagacagaaaggatgattgccatttgcactgagcttcgaaaggtaagtttcctttctctccacgctctctga

SEQ ID NO: 278 - DNA, 1869 bp, synthetic construct
  source 1..1869: mol_type = other DNA
atggcttcaaccaccttacgctcctcctcactattcttcggatccaacatcataatctcatcccccactcccaaaaccacaaaaccctcctccttccacgccgccttagcagcgatcctcttctcatcaatctctctccccttccccaaaagccctagctttagaccaacccctcccccccacccaacccatgtcataaccgcccccaacccgaaatcaacccaatcatcctccgatctccccgacgggtcccagtggcggtacagcgagttcctcaacgcggtgaagaaggggaaagtcgagagagtcaggttcagcaaagacggctccgttctccagctcaccgccgtcgataaccgccgcgcctccgtcgtggtccccaacgaccccgatctcatcgatatcctcgccatgaacggcgtcgacatctccgtctccgagggcgagtccggcaacgacctcttcaccatcatcggcaacttgattttccccatcctcgccttcggtgggcttttccttctcttcaggagggccggcggtgggcctggcgggcctggagggttagggtacactgccttgggcgccaagattcccaaagggtgtctccttgttgggcctcctggtactggtaagactctgttagctagagctgttgctggagaggcgggaccgttcttctcctgcgcggcttctgagtttgttgagcttttcgtcggtgtgggtgcgtctagggttagggatttgttcgagaaggcgaaggccaaggcgccttgcattgtttttatcgatgagattgatgctgtggggaggcagaggggagctgggatgggaggagggaatgatgagagggagcagacgattaatcagctgttgactgagatggatgggttctctggtaactctggtgtgattgttttggcggctacgaatagacctgatgtgttggactctgcgttgttgaggcctgggaggtttgataggcaggtgacggttgataggcctgatgttgctggcagagtcaagattcttcaggtttggttcattctagagggaaggcgattggtaaagatgtggactttgataaggttgcgaggaggactcctggtttcaccggctgctgacttgcagaacctgatgaatgaagccgcgattcttgcggcgaggcgtgagcttaaggagataagcaaggatgagatctctgatgcacttgagaggatcattgctggacctgagaagaagaatgctgttgtctctgatgagaagaagagacttgttgcattaccatgggctggtcatgctctagtgggtgctcttatgccagagtatgatcctgttgccaagatttccatcattcctcgtggccaagctggtgggcttaccttttttgctcccagtgaagaaagactcgagtctggattgtacagtagaagctacctcgagaatcagatggctgttgcacttggtggaaggagggttgcagaggaggtgatctttggagatgacaatgtgacgactggagcatcaaatgatttcatgcaggtgtctcgtgtggcacgtcaaatgatcgagaggttcgggttcagcaaaaagattggacaagttgccgttggtggccctggtggaaatcccttcatgggtcaacaacagatgtcttcacagaaagattactcaatggcaactgctgatatcgtagatgctgaggtgagagagctggtggagaaggcgtacaagagagccacggacatcataacaactcacattgatatcttgcacaagcttgcgcagctcttgatcgagaaagaaactgttgatggagaagagttcatgagtctctttattgacggccaagctgagttgtatgtttcttag

SEQ ID NO: 279 - protein, 461 aa, Raphanus raphanistrum
MAATTTTAASLACAHFLNQNKAYPFPSKTSVQVSQIIDGRKMRSATLFSAVSTDKTITTTAQSVSPTACDRVRRHTISVFVGDESGIINRIAGVFARRGYNIESLAVGLNEDKAMFTIVVLGTDKVLQQVVEQLNKLVNVIKVEDLSKEPHVERELMLIKLNADPTTRSEIMWLVDIFRAKVVDTSDHTLTIEVTGDPGKMVALTANLEKFGIKEVARTGKIALRREKMGETAPFWRFSAASYPHLVNQSSHENVAENTKRALTGGNGNASSGGDVYPVEPYNDFKPVLDAHWGVVYDEDSNGLRSHTLSILVANVPGVLNLITGAISRRGYNIQSLAVGPAEKEGLSRITTVIPGTDENIDKLVRQLQKLIDLHEVQNITHMPFAERELMLIKVAAHTSARRDVLDIAQVFRAKAIDVSDHTITLEVARTGRVVARTGRVALVRESGVDSTYLRGYSLPL

SEQ ID NO: 280 - protein, 474 aa, Raphanus raphanistrum
MPLAISLPTSSPPHLFPRRSRVHQNLRYRRVSIPRSSSTQENQTGVIIVGAGLAGLAAATRLASAQIPFLLLEASDGVGGRVRTDTTVDGFLLDRGFQIFITAYPEAKKLLDYEALDLRRFYAGAKVFYGGEFHTVADPLRHLWDSVASLANPIGSVVDKGLIALTRARVLVKSDEDILTSAEEVPTIDLLRKIGFSDEILDRFFRPFFGGIFFDRDLETTSKLFDFVFRCLALGENTLPAMGIGEIPNQLAAKLPVDSVLMNTRVDSIEYPNGSGSGPPIVRLQDGGVLSAELGVILAVELPQVDKLLNGIREPVRTKPARSTVCIYFAAEPNQIPVEDPVLFLNGSNTGIINNMFFPTNVARTYAPPGKALVSVSLIGSFEDRSDDDLAAEVIRELSGWFGESLVTSWRHLRTYRIQFAQPNQCPPTDLVKNPRVGSGLYLSGDYMTSATFDGALVSGRRAVEALLREKGLV

SEQ ID NO: 281 - protein, 566 aa, Raphanus raphanistrum
MVVFGNVSAANLPYQNGFLKAISSGGCDLMGHRSFKISTCSQALKTRTRRSAGPLKVVCVDIPRPELENTVNFLEAASLSASFRSAPRPAKPLKVVIAGAGLAGLSTAKYLADAGHIPLLLEARDVLGGKIAAWKDEDGDWYETGLHIFFGAYPNVQNLFGELGINDRLQWKEHSMIFAMPSKPGEFSRFDFPDVLPAPLNGIWAILRNNEMLTWPEKIKFAIGLLPAMVGGQAYVEAQDGLSVEQWMRKQGVPDRVTDEVFIAMSKALNFINPDELSMQCILIALNRFLQEKHGSKMAFLDGNPPERLCTPIVEHIRSLGGEVRLNSRIKKIELDENDATVKSFLLTDGTTIQGDAYVFATPVDILKLLLPDSWKEIPYFKRLEKLVGVPVINVHIWFDRKLKNTYDHLLFSRSNLLSVYADMSLTCKEYYDPNRSMLELVFAPAEEWISRTDSDIIDATMEELERLFPDEISADQSKAKILKYHVVKTPRSVYKTIPDCEPCRPLQRSPIKGFYLAGDYTKQKYLASMEGAVLSGKFCSQSILQDYEMLAASGPQKLSEATVST

SEQ ID NO: 282 - protein, 452 aa, Raphanus raphanistrum
MAQASRICHVPCLNISNLSKSNQSKSPFSLSLKTTQQQPPRASSWGLKKSVMTLTRPTVKVTASSVSTAEKAASEIVLQPIREISGLIKLPGSKSLSNRILLLAALSEGTTVVDNLLNSDDINYMLDALKKLGLNVERDSANNRAVVEGCGGIFPASLDAKGDIELYLGNAGTAMRPLTAAVTAAGGNASYVLDGVPRMRERPIGDLVVGLKQLGADVECTLGTNCPPVRVNANGGLPGGKVKLSGSISSQYLTALLMAAPLALGDVEIEIVDKLISVPYVEMTLKLMERYGVSAEHSDSWDRFFVKGGQKYKSPGNAYVEGDASSASYFLAGAAITGETVTVEGCGTTSLQGDVKFAEVLEKMGCKVSWTENSVTVTGPSRDAFGMRHLRAVDVNMNKMPDVAMTLAVVALFADGPTTIRDVASWRVKETERMIAICTELRKVSFLSLHAL

SEQ ID NO: 283 - protein, 622 aa, Raphanus raphanistrum
MASTTLRSSSLFFGSNIIISSPTPKTTKPSSFHAALAAILFSSISLPFPKSPSFRPTPPPHPTHVITAPNPKSTQSSSDLPDGSQWRYSEFLNAVKKGKVERVRFSKDGSVLQLTAVDNRRASVVVPNDPDLIDILAMNGVDISVSEGESGNDLFTIIGNLIFPILAFGGLFLLFRRAGGGPGGPGGLGYTALGAKIPKGCLLVGPPGTGKTLLARAVAGEAGPFFSCAASEFVELFVGVGASRVRDLFEKAKAKAPCIVFIDEIDAVGRQRGAGMGGGNDEREQTINQLLTEMDGFSGNSGVIVLAATNRPDVLDSALLRPGRFDRQVTVDRPDVAGRVKILQVWFILEGRRLVKMWTLIRLRGGLLVSPAADLQNLMNEAAILAARRELKEISKDEISDALERIIAGPEKKNAVVSDEKKRLVALPWAGHALVGALMPEYDPVAKISIIPRGQAGGLTFFAPSEERLESGLYSRSYLENQMAVALGGRRVAEEVIFGDDNVTTGASNDFMQVSRVARQMIERFGFSKKIGQVAVGGPGGNPFMGQQQMSSQKDYSMATADIVDAEVRELVEKAYKRATDIITTHIDILHKLAQLLIEKETVDGEEFMSLFIDGQAELYVS

SEQ ID NO: 284 - DNA, 475 bp, synthetic construct
  source 1..475: mol_type = other DNA
gcttcctcgtggggattgaagaagagtgtgatgacgttaactcgtcccaccgtcaaggtaacagcttcctccgtctccacggcggagaaagcagcgtcggagattgtgcttcaacccatcagagaaatctcgggtcttatcaagctacccggatccaaatctctctccaatcggatccttcttctcgccgctctatctgagggaactactgtagtggacaacttgttgaacagtgatgacatcaactacatgctcgatgcgttgaagaagctggggcttaacgtggagcgtgacagtgcaaacaaccgtgctgttgtcgaaggatgcggcgggatcttcccagcttccttggatgccaagggtgatatcgagctgtaccttgggaatgcaggaacagccatgcgtccacttaccgctgctgttactgctgcaggtggcaacgcaagttatgtgcttgatggggtgcctaggatga

SEQ ID NO: 285 - DNA, 1192 bp, synthetic construct
  source 1..1192: mol_type = other DNA
gaattctaatacgactcactatagcatgtagttgatgtcatcactgttcaacaagttgtccactacagtagttccctcagatagagcggcgagaagaaggatccgattggagagagatttggatccgggtagcttgataagacccgagatttctctgatgggttgaagcacaatctccgacgctgctttctccgccgtggagacggaggaagctgttaccttgacggtgggacgagttaacgtcatcacactcttcttcaatccccacgaggaagcgcgcaagctagcaggatctgccatgtcccatgtcttaatatctccaatctctccaaatcaaaccaaagcaaatcacccttctccctctcgctgaagacaacgcagcagcagcctcctcgagcttcctcgtgggattgaagaagagtgtgatacgttaactcgtcccaccgtcaggtaacagcttcctccgtctcacggcggagaaagcagcgtcgagattgtgcttcaacccatcgagaaatctcgggtcttatcagctacccggatccaaatctcttccaatcggatccttcttctcccgctctatctgagggaactatgtagtggacaacttgttgaaagtgatgacatcaactacatgctcgatgcgttgaaaagctggggcttaacgtggaggtgacagtgcaaacaaccgtgtgttgtcgaaggatgcggcggatcttcccagcttccttggatccaagggtgatatcgagctgtccttgggaatgcaggaacagcatgcgtccacttaccgctgctttactgctgcaggtggcaacgaagttatgtgcttgatggggtcctaggatgagggaaagacctataggagatttggttgttggtcttaaacagcttggtgctgatgttgagtgtactctgggaactaactgccctcctgttcgtgtcaatgctaacggtggccttcccggttcatcctaggcaccccatcaagcacataacttgcgttgccacctgcagcagtaacagcagcggtaagtggacgcatggctgttcctgcattcccaaggtacagctcgatatcacccttggcatccaaggaagctgggaagatcccgccgcatccttcgacaacagcacggttgtttgcactgtcacgctccacgttaagccccagcttcttcaacgcatcgag